(19) United States
(12) Patent Application Publication
(10) Pub. No.: US 2026/0174885 A1
(43) Pub. Date: Jun. 25, 2026

(54) POLYPLEXES OF NUCLEIC ACIDS AND TARGETED CONJUGATES COMPRISING POLYETHYLENEIMINE AND POLYETHYLENE GLYCOL

(71) Applicant: TARGIMMUNE THERAPEUTICS AG, Basel (CH)

(72) Inventors: Eric KITAS, Aesch (CH); Maya ZIGLER, Basel (CH); Esteban POMBO-VILLAR, Binningen (CH)

(21) Appl. No.: 19/127,016

(22) PCT Filed: Nov. 7, 2023
(86) PCT No.: PCT/EP2023/081001

§ 371 Date: May 2, 2025

(30) Foreign Application Priority Data
Nov. 7, 2022 (WO) PCT/EP2022/080986
May 11, 2023 (EP) 23172849.4

(51) Int. Cl. A61K 47/68; A61K 47/10; A61K 47/60; C07D 471/04

(52) U.S. Cl. CPC A61K 47/6803; A61K 47/10; A61K 47/60; C07D 471/04; C07K 2317/92

(57) ABSTRACT

The present invention provides targeting polyplexes comprised of (i) nucleic acids, in particular nucleic acids encoding pharmaceutically active peptides or proteins, and (ii) targeting conjugates comprising LPEI and PEG fragments that are connected by discrete linkages formed through defined, chemoselective reactions. Thus, the LPEI fragment is bonded in a linear end- to-end fashion to a single PEG fragment. The linear conjugates are further conjugated to a targeting fragment to enable selective interaction with a particular cell type. The polyplexes selectively deliver the nucleic acids to the targeted cells resulting in high expression and efficient protein translation as well as secretion of the encoded pharmaceutically active proteins.

### RELATED ART

Cancer remains a leading cause of death world-wide. For most solid tumours after surgical removal, chemotherapy is a key treatment option for managing the remaining cancer cells. A main reason for failure of chemotherapy is inefficient targeting and uptake of the chemotherapeutic agent by the tumour (J K Vasir and V Labhasetwar, Technology in Cancer Research & Treatment, 2005, 4(4):363-374). Poor accessibility to the tumour requires higher doses, and due to the nature of the chemotherapeutic agent this results in non-specific uptake and toxicity of healthy cells. A targeted drug delivery strategy whereby the therapeutic agent is reversibly bound to a targeting ligand and selectively delivered to a cell for treatment is now applied to many chemotherapeutics agents in clinical use. This strategy has shown promise to maximize the safety and efficacy of a given chemotherapeutic agent, as their selective delivery into target cells avoids the nonspecific uptake and associated toxicities to healthy cells (M Srinivasarao and PS Low, Chem Rev, 2017, 117:12133-12164) that can result in higher maximum tolerated doses.

In case of nucleic acid therapeutics, including DNA and mRNA, nanoparticle delivery systems have attracted a lot of interest in particular due to their applications in cancer immunotherapy (AJ Mukalel et al., 2019, Cancer Lett. 458:102-112; U Laechelt and E Wagner, 2015 Chem Rev 115(19):11043-78; R S Riley et al, 2019, Nat Rev Drug Discov 18(3):175-196; X Tan et al., 2020, J Control Release 323:240-252; and references cited therein). However, these nucleic acid therapeutics must also overcome numerous delivery obstacles to be successful, including rapid in vivo degradation, poor uptake in target cells, required nuclear entry, and potential in vivo toxicity in healthy cells and tissues. Nanoparticle delivery systems including targeted nanoparticle delivery systems have been engineered to address and try to overcome several of these barriers as a means to safely and effectively deliver nucleic acid therapeutics (DE Large et al, 2018, Adv Therap, 1800091; A Patel et al, 2020, BioDrugs 34:273-293; Hj Vaughan et al, 2020, Adv Mater, 32(13):e1901081).

Cationic polymers are known to form polyplexes with negatively charged nucleic acids in solution. For example, linear polyethyleneimine (LPEI) is protonated at physiological pH and therefore carries a net positive charge. When LPEI is incubated with a nucleic acid, which carries a net negative charge at physiological pH, LPEI and the nucleic acid can form polyplexes that are held together by electrostatic interaction. These polyplexes can be taken up by cells in vivo where they can deliver the nucleic acid sequences intracellularly. Accordingly, polyplexes comprising cationic polymers and nucleic acids can be used as vectors for therapy. Despite their promise, technical challenges have arisen related to forming homogenous and well-characterized cationic polymers. Polyplexes comprising only LPEI can be prone to aggregation and interaction with serum proteins, limiting their potential as nucleic acid delivery agents. To overcome these challenges, polymeric LPEI can be conjugated to polyethylene glycol (PEG). The PEG fragment can help shield the LPEI from the surrounding matrix and improve the biocompatibility and blood circulation of the resulting polyplexes. Examples of such polyethyleneimine-polyethylene glycol conjugates further comprising a targeting moiety as non-viral vectors for delivering in particular double stranded RNAs such as polyinosinic:polycytidylic acid have been described (WO2015/173824; WO2010/073247; US2004/248842A1; Vetter V C, Wagner E. J Control Release, 2022 346:110-135; and references cited therein). However, the coupling of PEG to LPEI within the referenced conjugates and vectors takes place by formation of covalent bonds between electrophilic PEG fragment(s) and the secondary amines embedded within the LPEI backbone fragment, and thus leads to branched, heterogenous conjugates and vectors with random and not defined inclusion of PEG fragments that are characterized on the basis of average PEG inclusion density. In such conjugates, a broad range of variable numbers of PEG fragments are bonded orthogonally to the LPEI fragment with no site specificity. Such random synthesis and imprecise characterization of the LPEI-PEG conjugates can make it difficult to establish clear structure-activity relationships (SAR) between the structure of the conjugates and the activity of the resulting polyplex.

Despite the recent efforts and achievements, there is still a great interest in developing novel targeted delivery platforms that can protect nucleic acids, including mRNAs, as well as mediate their delivery into the desired tissues and cells in order to exploit the powerful therapeutic potential of these molecules (AJ Mukalel et al., 2019, Cancer Lett. 458:102-112). Accordingly, there is a need for homogenous nanoparticles, in particular for homogeneous LPEI-PEG conjugates with well-defined chemical structures, capable of selective delivery of nucleic acids including mRNAs or pDNAs to the desired tissues and cells.

### SUMMARY OF THE INVENTION

The present invention provides targeting polyplexes comprised of (i) nucleic acids encoding peptides or proteins of interest, in particular encoding pharmaceutically active peptides or proteins such as cytokines, interferons, or toxins, and (ii) targeting conjugates comprising LPEI and PEG fragments that are connected by discrete linkages formed through defined, chemoselective reactions instead of through random and uncontrolled bonding of an electrophilic PEG fragment to multiple nucleophiles of an LPEI backbone fragment. Thus, the present invention provides more homogenous targeting conjugates with defined chemical structures. The discrete linkages not only ensure consistent and predictable ratios of LPEI to PEG fragments, but further ensure defined linear instead of random branched conjugates. Thus, the LPEI fragment is bonded in a linear end-to-end fashion to a single PEG fragment.

The conjugates further comprise targeting fragments linked to the PEG fragments which allow to target a particular cell type and to facilitate the uptake of the inventive compositions and pharmaceutically active nucleic acids in said particular cell type. Thus, preferred embodiments comprise targeting fragments such as hEGF, DUPA or folate specifically connected to the LPEI-PEG diconjugates to target the corresponding receptors such hEGFR, PSMA or folate receptor on the particular cell types, typically cancer cell types, on which said receptors show high expression and are overexpressed.

Further advantageously and surprisingly, the inventors have found that the resulting preferred conjugates and polyplexes in accordance with the present invention which have a significant reduced heterogeneity due to the defined chemoselective bonding of the LPEI fragments to the PEG fragments, and thus, which have a significant reduced number of potentially biologically active conjugates and polyplexes, not only form polyplexes of suitable size, but also maintain or even increase their overall biological activity such as highly selective targeted delivery of the pharmaceutically active nucleic acids as well as the subsequent efficient translation and secretion of the encoded pharmaceutically active proteins. Thus, the inventive compositions and polyplexes do not only selectively deliver pharmaceutically active nucleic acids encoding pharmaceutically active peptides or proteins to the targeted cells, in particular cancer cells, but furthermore, said delivery results in high expression and efficient protein translation as well as secretion of the encoded pharmaceutically active proteins.

Thus, in one aspect, the present invention provides a composition comprising a polyplex, wherein said polyplex comprises a conjugate and a nucleic acid, wherein said nucleic acid is preferably non-covalently bound to said conjugate, and wherein said conjugate comprises:
a linear polyethyleneimine fragment comprising an alpha terminus and an omega terminus; wherein the alpha terminus of said polyethyleneimine fragment is an initiation residue;
a polyethylene glycol fragment comprising a first terminal end and a second terminal end; and
wherein the omega terminus of the polyethyleneimine fragment is connected to the first terminal end of the polyethylene glycol fragment by a divalent covalent linking group —Z—X¹—, wherein —Z—X¹ is not a single bond and —Z— is not an amide;
wherein the second terminal end of the polyethylene glycol fragment is connected to a targeting fragment by a divalent covalent linking moiety X², and
   wherein said nucleic acid is a pharmaceutically active nucleic acid, wherein said pharmaceutically active nucleic acid is a nucleic acid that encodes a pharmaceutically active peptide or protein. In a preferred embodiment of this aspect, said composition consists of said polyplex.

In another aspect, the present invention provides a composition comprising a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

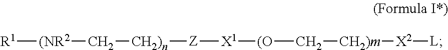

wherein
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ— is H;
X¹ and X² are independently divalent covalent linking moieties;
Z is a divalent covalent linking moiety wherein Z is not a single bond and Z is not —NHC(O)—;
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein, and wherein preferably said composition consists of said polyplex.

In another aspect, the present invention provides a polyplex, wherein said polyplex comprises a conjugate of the Formula I* or a pharmaceutically acceptable salt, solvate, hydrate, tautomer or enantiomer thereof, and a nucleic acid, wherein said nucleic acid is preferably non-covalently bound to said conjugate:

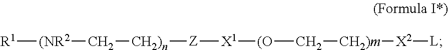

wherein
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ- is H;
X¹ and X² are independently divalent covalent linking moieties;
Z is a divalent covalent linking moiety wherein Z is not a single bond and Z is not —NHC(O)—;
L is a targeting fragment, wherein preferably said targeting fragment is capable of binding to a cell, and wherein further preferably said targeting fragment is capable of binding to a cell surface receptor,
wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

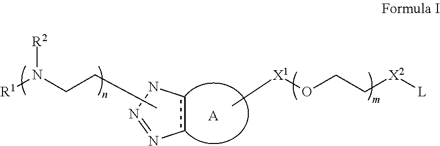

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ- is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

Although the N—N═N fragment of bicyclic ring in Formula I is typically drawn herein using one single bond and one double bond for simplicity, one of skill in the art knows that Formula I and associated conjugate structures as depicted herein can alternatively be drawn as shown below. Such depictions and descriptions of Formula I are interchangeably used herein:

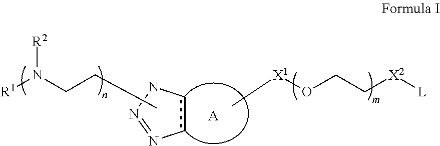

wherein the fragment

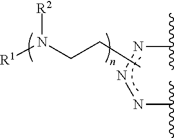

represents two different regioisomeric attachments of the fragment R¹(NR²CH₂CH₂)ₙ, i.e.

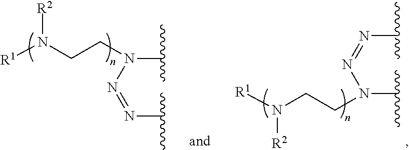

wherein the wavy lines represent chemical bonds to Ring A. Accordingly, Formula I as drawn herein encompasses two regioisomeric embodiments, i.e., wherein the fragment R¹(NR²CH₂CH₂)ₙ is bonded at the top nitrogen atom in the structures above or at the bottom nitrogen atom in the structures above, but not at the middle nitrogen atom. One of skill in the art knows that the same applies to other formulae herein, including Formula IA, Formula IB, Formula IC, Formula ID, Formula IE, Formula IH, Formula IJ, Formula IK and the like.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

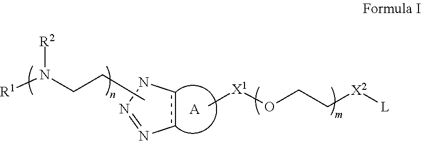

wherein:
[symbol] is a single bond or a double bond;
n is any integer between 1 and 1500, preferably any integer between 2 and 1500;
m is any integer between 2 and 200, preferably any integer between 1 and 200, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)— is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

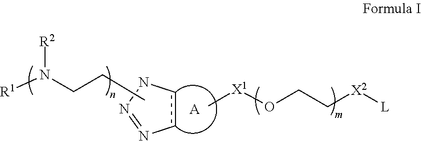

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ-moieties is H;
Ring A is a 5 to 10-membered cycloalkyl, cycloalkenyl, heterocycloalkyl, or heterocycloalkenyl, optionally substituted at any position with one or more R^{A1};
R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2};
R^{A2} is independently selected from C₁-C₆ alkyl, C₁-C₆ alkoxy, halogen —SO₃H, or —OSO₃H;
X¹ is a linking moiety of the formula —(Y¹)ₚ—, wherein p is an integer between 1 and 20, and each occurrence of Y¹ is independently selected from a chemical bond, —CR¹¹R¹²—, —C(O)—, —O—, —S—, —NR¹³—, an amino acid residue, a divalent phenyl moiety, a divalent heterocycle moiety, and a divalent heteroaryl moiety, wherein each divalent phenyl or heteroaryl is optionally substituted with one or more R¹³, and each divalent heterocycle is optionally substituted with one or more R¹⁴; wherein R¹¹, R¹² and R¹³ are independently, at each occurrence, H or C₁-C₆ alkyl; and wherein R¹⁴ is independently, at each occurrence, H, C₁-C₆ alkyl, or oxo;
X² is a linking moiety of the formula —(Y²)_{q}—, wherein q is an integer between 1 and 50, and each occurrence of Y² is independently selected from a chemical bond, —CR²¹R²²—, NR²³—, —O—, —S—, —C(O)—, an amino acid residue, a divalent phenyl moiety, a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴; wherein R²¹, R²², and R²³ are each independently, at each occurrence, —H, —CO₂H, or C₁-C₆ alkyl, wherein each C₁-C₆ alkyl is optionally substituted with one or more —OH, oxo, C₆-C₁₀ aryl, or 5 to 8-membered heteroaryl; and wherein R²⁴ is independently, at each occurrence, —H, —CO₂H, C₁-C₆ alkyl, or oxo; and
L is a targeting fragment preferably capable of binding to a cell, and wherein preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein, and wherein further preferably said composition consists of said polyplex.

In another aspect, the present invention provides a polyplex, wherein said polyplex comprises a conjugate of the Formula I, or a pharmaceutically acceptable salt, solvate, hydrate, tautomer or enantiomer thereof, and a nucleic acid, wherein said nucleic acid is preferably non-covalently bound to said conjugate:

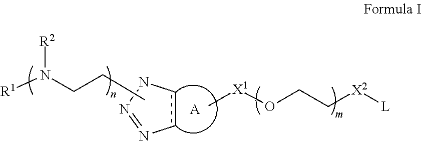

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ-moieties is H;
Ring A is a 5 to 10-membered cycloalkyl, cycloalkenyl, heterocycloalkyl, or heterocycloalkenyl, optionally substituted at any position with one or more R^{A1};
R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2};
R^{A2} is independently selected from C₁-C₆ alkyl, C₁-C₆ alkoxy, halogen —SO₃H, or —OSO₃H;
X¹ is a linking moiety of the formula —(Y¹)ₚ—, wherein p is an integer between 1 and 20, and each occurrence of Y¹ is independently selected from a chemical bond, —CR¹¹R¹²—, —C(O)—, —O—, —S—, —NR¹³—, an amino acid residue, a divalent phenyl moiety, a divalent heterocycle moiety, and a divalent heteroaryl moiety, wherein each divalent phenyl or heteroaryl is optionally substituted with one or more R¹³, and each divalent heterocycle is optionally substituted with one or more R¹⁴; wherein R¹¹, R¹² and R¹³ are independently, at each occurrence, H or C₁-C₆ alkyl; and wherein R¹⁴ is independently, at each occurrence, H, C₁-C₆ alkyl, or oxo;
X² is a linking moiety of the formula —(Y²)_{q}—, wherein q is an integer between 1 and 50, and each occurrence of Y² is independently selected from a chemical bond, —CR²¹R²²—, NR²³—, —O—, —S—, —C(O)—, an amino acid residue, a divalent phenyl moiety, a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴; wherein R²¹, R²², and R²³ are each independently, at each occurrence, —H, —CO₂H, or C₁-C₆ alkyl, wherein each C₁-C₆ alkyl is optionally substituted with one or more —OH, oxo, C₆-C₁₀ aryl, or 5 to 8-membered heteroaryl; and wherein R²⁴ is independently, at each occurrence, —H, —CO₂H, C₁-C₆ alkyl, or oxo; and
L is a targeting fragment preferably capable of binding to a cell and wherein preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In a preferred embodiment, said nucleic acid is a RNA. In another preferred embodiment, said nucleic acid is a single stranded RNA (ssRNA). In a further preferred embodiment, said ssRNA is a messenger RNA (mRNA).

In another preferred embodiment, said nucleic acid is a DNA. In a further preferred embodiment, said DNA is a plasmid DNA.

In one aspect, the present invention provides a pharmaceutical composition comprising a composition, wherein said composition comprises a polyplex, wherein said polyplex comprises a triconjugate, preferably said conjugate of Formula I* or of Formula I, and a nucleic acid, wherein said nucleic acid is preferably non-covalently bound to said conjugate, as described herein, wherein said nucleic acid is a pharmaceutically active nucleic acid, wherein said pharmaceutically active nucleic acid is a nucleic acid that encodes a pharmaceutically active peptide or protein, and a pharmaceutically acceptable salt thereof.

In one aspect, the present invention provides a composition or a polyplex as described herein, or a pharmaceutical composition comprising said composition or said polyplex as described herein for use in the treatment of a disease or disorder, preferably of a cancer.

In one aspect, the present invention provides the use of a composition or a polyplex as described herein, or a pharmaceutical composition comprising said composition or said polyplex as described herein, for use in the manufacture of a medicament for the treatment of a disease or disorder such as a cancer.

In another aspect, the present invention provides a method of treating a disease or disorder such as a cancer in a subject in need thereof, the method comprising administering to the subject an effective amount of a composition or a polyplex as described herein, or a pharmaceutical composition comprising said composition or said polyplex as described herein.

The linear, nonrandom LPEI-PEG diconjugates described herein, and thus the inventive compositions and polyplexes comprising the triconjugates with the targeting fragments linked to the linear, nonrandom LPEI-PEG diconjugates, not only ensure consistent and predictable ratios of LPEI to PEG fragments, but typically and preferably further ensure structurally defined linear conjugates of LPEI fragment to PEG fragment. Thus, they offer greater batch-to-batch consistency, ease of manufacturing, and more predictable SAR compared with the branched LPEI-PEG diconjugates currently prepared using the random, uncontrolled synthesis strategies described above.

Further advantageously and surprisingly, when the inventive linear, nonrandom conjugates described herein are combined with pharmaceutically active nucleic acids such as mRNA or plasmid DNA (pDNA) encoding pharmaceutically active peptides or proteins such as cytokines, interferons, or toxins, to form polyplexes and administered to cells, the polyplexes surprisingly not only maintain, but may even increase their biological activity as compared to the respective polyplexes made using random, branched conjugates. Thus, despite the significant reduction of variability and number in structures of conjugates, and thus significant reduction of variability and number in structures of possible biological activity including targeting and presenting their targeting fragments to the surface of the targeted cells as well subsequent uptake, and translation as well as secretion of the encoded pharmaceutically active proteins, there is no loss in efficacy of the linear and inventive polyplexes described herein. To the contrary, the inventive compositions and polyplexes are even able to increase their overall biological activity.

Additional features and advantages of the present technology will be apparent to one of skill in the art upon reading the Detailed Description of the Invention, below and further aspects and embodiments of the present invention will be become apparent as this description continues.

### BRIEF DESCRIPTION OF FIGURES

FIG. 1 is a DLS back scatter plot taken in triplicate of a LPEI-l-[N₃:DBCO]-PEG₃₆-DUPA:DT-A) polyplex measuring size distribution and ζ-potential in 20 mM HEPES, 5% glucose at pH 7.2, 0.1 mg/mL in 5% glucose, 1.0 mL volume, N/P ratio of 4. The z-average diameter was 103.4 nm with a polydispersity index (PDI) of 0.197. The ζ-potential was 44.5 mV.

FIG. 2A is a plot of luminescence (AU) in Renca parental cells and Renca EGFR M1 H cells treated with LPEI-l-[N₃:DBCO]PEG₃₆-hEGF:[Fluc mRNA] compared to the control delivery vehicle Messenger MAX. The luminescence was measured at N/P ratios of 4, 6 and 12, and at final concentrations from 0.125 to 1.0 μg/mL of LPEI-l-[N₃:DBCO]PEG₃₆-hEGF:[Fluc mRNA] and lipofectamine Messenger MAX at 24 hours after treatment.

FIG. 2B is a plot of luminescence (AU) in Renca parental cells and Renca EGFR M1 H cells treated with LPEI-l-[N₃:DBCO]PEG₃₆-hEGF:[Fluc mRNA] compared to the control delivery vehicle jetPEI. The luminescence was measured at N/P ratios of 4, 6 and 12, and at final concentrations from 0.125 to 1.0 μg/mL of LPEI-l-[N₃:DBCO]PEG₃₆-hEGF:[Fluc mRNA] and jetPEI at 24 hours after treatment.

FIG. 2C is a plot of the ratio of luminescence (AU) between Renca parental cells and Renca EGFR M1 H cells treated with LPEI-l-[N₃:DBCO]PEG₃₆-hEGF:[Fluc mRNA] with Messenger MAX as a comparison delivery vehicle. The luminescence was measured at N/P ratios of 4, 6 and 12, and at final concentrations from 0.125 to 1.0 μg/mL of LPEI-l-[N₃:DBCO]PEG₃₆-hEGF:[Fluc mRNA] and lipofectamine Messenger MAX at 24 hours after treatment. The ratio was calculated by dividing the luminescence signal from RencaEGFR M1 H cells by the luminescence signal from Renca parental cells.

FIG. 2D is a plot of the ratio of luminescence (AU) between Renca parental cells and Renca EGFR M1 H cells treated with LPEI-l-[N₃:DBCO]PEG₃₆-hEGF:[Fluc mRNA] with jetPEI as a comparison delivery vehicle. The luminescence was measured at N/P ratios of 4, 6 and 12 and at final concentrations from 0.125 to 1.0 μg/mL of LPEI-l-[N₃:DBCO]PEG₃₆-hEGF:[Fluc mRNA] and jetPEI at 24 hours after treatment. and the ratio was calculated by dividing the average luminescence signal from RencaEGFR M1 H cells by the average luminescence signal from Renca parental cells.

FIG. 2E is a plot of percent survival in Renca parental cells and Renca EGFR M1 H cells treated with LPEI-l-[N₃:DBCO]PEG₃₆-hEGF:[Fluc mRNA] compared to the control delivery vehicle Messenger MAX. The percent survival was measured at N/P ratios of 4, 6 and 12, and at final concentrations from 0.125 to 1.0 μg/mL of LPEI-l-[N₃:DBCO]PEG₃₆-hEGF:[Fluc mRNA] and Messenger MAX 24 hours after treatment.

FIG. 3A shows relative luminescence (AU) in Renca parental cells and Renca EGFR M1 H cells treated with LPEI-l-[N₃:DBCO]PEG₃₆-hEGF:[Fluc mRNA]6 hours after treatment at an N/P ratio of 4.

FIG. 3B shows relative luminescence (AU) in Renca parental cells and Renca EGFR M1 H cells treated with LPEI-l-[N₃:DBCO]PEG₃₆-hEGF:[Fluc mRNA]6 hours after treatment at an N/P ratio of 6.

FIG. 3C shows relative luminescence (AU) in Renca parental cells and Renca EGFR M1 H cells treated with LPEI-l-[N₃:DBCO]PEG₃₆-hEGF: [Fluc mRNA]22 hours after treatment at an N/P ratio of 4.

FIG. 3D shows relative luminescence (AU) in Renca parental cells and Renca EGFR M1 H cells treated with LPEI-l-[N₃:DBCO]PEG₃₆-hEGF: [Fluc mRNA] at 22 hours after treatment at an N/P ratio of 6.

FIG. 3E shows luminescence (AU) from different densities (500-20,000 cells/well) of B16F10-hEGFR cells transfected with LPEI-l-[N₃:DBCO]PEG₃₆-hEGF:[Fluc mRNA] at an N/P ratio of 6 for 24 h.

FIG. 4 depicts luminescence normalized to survival in human prostate cell lines with differential cell surface expression of PSMA: PSMA high expressing LNCaP cells, and PSMA low expressing DU145 cells following transfection with PSMA targeting polyplexes containing mRNA encoding Luciferase. The X axis indicates the concentration of the mRNA in the polyplexes (0.25, 0.5 and 1.0 μg/mL). The Y axis indicates luminescence normalized to survival in arbitrary units (AU). Selective transfection of PSMA overexpressing cells with Luc mRNA as well as selective expression of Luciferase was demonstrated.

FIG. 5 depicts luminescence from cancer cells with differential cell surface expression of human folate receptor (FR) (MCF7: low folate receptor expression; SKOV3: high folate receptor expression) following treatment with FR targeting polyplexes containing mRNA encoding Renilla luciferase (R-Luc). The X axis indicates the concentration of the mRNA in the polyplexes (0.125, 0.25, 0.5 and 1.0 μg/mL). The Y axis indicates luminescence in arbitrary units (RLUs). Standard deviation from the quadruplicate samples is presented. Selective expression of Renilla Luc in folate receptor overexpressing cells is demonstrated.

FIG. 6 depicts the levels of secreted human IL-2 normalized to survival from two cell lines with differential human EGFR (hEGFR) expression: hEGFR high expressing RencaEGFR M1 H cells and human EGFR negative Renca (parental) following transfection with EGFR targeting polyplexes containing hIL-2 mRNA. The X axis indicates the concentration of the mRNA in the polyplexes (0.125, 0.25, 0.5 and 1.0 μg/mL). The Y axis indicates the levels of secreted human IL-2 normalized to survival in arbitrary units (AU). Selective expression and secretion of human IL-2 from EGFR high expressing cells is demonstrated.

FIG. 7 depicts the levels of secreted human IL-2 from two cell lines with differential PSMA expression: PSMA high expressing LNCaP cells, and PSMA low expressing DU145 cells following transfection with PSMA targeting polyplexes containing hIL-2 mRNA. Selective expression of human IL-2 from PSMA overexpressing cells is demonstrated.

FIG. 8 depicts the levels of secreted human IFNβ from two cell lines with differential PSMA expression: high PSMA expressing LNCaP cells, and low PSMA expressing DU145 cells following transfection with PSMA targeting polyplexes containing hIFNβ mRNA. Selective expression of human IFNβ from high PSMA expressing cells is demonstrated.

FIG. 9 depicts the levels of secreted human IFNγ (hIFNγ) from RencaEGFR M1 H (high expression of human EGFR) and Renca (parental, no expression of human EGFR negative) cell lines following transfection with EGFR targeting polyplexes containing hIFN7 mRNA. Selective transfection of EGFR overexpressing cells with hIFNγ mRNA and selective expression and secretion of hIFNγ protein is demonstrated.

FIG. 10 depicts the levels of human EPO secreted by cancer cells with differential expression of human folate receptor (FR) (SKOV3: high FR expression; MCF7: low FR expression) following treatment with FR targeting polyplexes containing mRNA encoding human EPO. The X axis indicates the concentration of the mRNA in the polyplexes (0.125, 0.25, 0.5 and 1.0 μg/mL). The Y axis indicates the concentration of hEPO released in the medium (mIU/mL). Standard deviation from the quadruplicate samples is presented. Selective expression of hEPO in folate receptor overexpressing cells is demonstrated.

FIG. 11 depicts protein biosynthesis inhibition by DT-A protein in two cell lines with differential PSMA expression: high PSMA-expressing LNCaP cells, and low PSMA-expressing DU145 cells following transfection with PSMA targeting polyplexes LPEI-1-[N₃:DBCO]PEG₃₆-DUPA containing mRNA DT-A. Western blot analysis with an anti-puromycin antibody as probe was utilized to detect inhibition of protein biosynthesis. GAPDH was used as a loading control. Selective inhibition of protein biosynthesis in PSMA overexpressing cells is demonstrated.

FIG. 12A depicts cell surface expression of human EGFR on various cell lines: RencaEGFR M1 H, WI-38, and MCF-7 cells. Data shown in FIG. 12A and FIG. 12B are from two separate experiments using different flow cytometers.

FIG. 12B depicts cell surface expression of human EGFR on various cell lines: WI-38, U87MG and MCF-7 cells. Data shown in FIG. 12A and FIG. 12B are from two separate experiments using different flow cytometers.

FIG. 12C depicts the levels of luminescence normalized to cell survival from high EGFR-expressing RencaEGFR M1 H cells and low EGFR expressing MCF7 cells, following transfection with EGFR-targeting polyplexes containing LPEI-l-[N₃:DBCO]PEG₃₆-hEGF and a plasmid encoding luciferase formulated at N/P ratio of 6. Selective expression and activity of luciferase in EGFR overexpressing cells is demonstrated.

FIG. 12D depicts the levels of luminescence normalized to cell survival in additional cell lines: rapidly proliferating cancerous U87MG cells, which express moderate levels of EGFR; slowly proliferating non-cancerous WI38 cells, which also express moderate levels of EGFR; and slowly proliferating non-cancerous HUVEC cells, which express minimal to no EGFR. These cells were transfected with EGFR-targeting polyplexes containing LPEI-l-[N₃:DBCO]PEG₃₆-hEGF and luciferase-encoding plasmid (N/P ratio of 6) in the same experiment as the cells shown in FIG. 12C. Selective expression of luciferase in rapidly proliferating cancer cells expressing moderate levels of EGFR is demonstrated.

FIG. 13A depicts the levels of luminescence in two cell lines with differential human EGFR expression, namely high EGFR-expressing RencaEGFR M1 H cells and human EGFR negative Renca (parental) cells, following transfection with the inventive linear EGFR-targeting polyplexes containing LPEI-l-[N₃:DBCO]PEG₃₆-hEGF and a plasmid that encodes luciferase (pGreenFire1-CMV) produced at N/P ratio of 3. Selective expression of luciferase in EGFR-overexpressing cells is demonstrated.

FIG. 13B depicts the levels of luminescence in two cell lines with differential human EGFR expression, namely high EGFR-expressing RencaEGFR M1 H cells and human EGFR negative Renca (parental) cells, following transfection with the inventive linear EGFR-targeting polyplexes containing LPEI-l-[N₃:DBCO]PEG₃₆-hEGF and a plasmid that encodes luciferase (pGreenFire1-CMV) produced at N/P ratio of 4. Selective expression of luciferase in EGFR-overexpressing cells is demonstrated.

FIG. 13C shows selective luminescence from B16F10-hEGFR cells. B16F10-hEGFR and B16F10 parental cells were treated with LPEI-l-[N₃:DBCO]PEG₃₆-hEGF:[pSZL] at an N/P ratio of 3 and 6. Luminescence and survival was determined after 6 days. Data is presented as relative luminescence normalized to survival.

FIG. 14 depicts luminescence from human prostate cell lines with differential cell surface expression of PSMA: high-PSMA expressing LNCaP cells, and low PSMA-expressing DU145 cells. The cells were treated with PSMA-targeting polyplexes containing LPEI-l-[N₃:DBCO]PEG₃₆-DUPA and plasmid DNA encoding luciferase. The X axis indicates the concentration of the pGreenFire-CMV in the polyplexes (0.25, 0.5 and 1.0 μg/mL). The Y axis indicates luminescence in arbitrary units (AU). Average and standard deviation from triplicate samples are presented. Selective expression of luciferase after transfection of PSMA overexpressing cells with plasmid DNA encoding luciferase (pGreenFire-CMV) is demonstrated.

FIG. 15A depicts the levels of secreted human IL-2 (hIL-2) from two cell lines with differential human EGFR expression: high EGFR-expressing RencaEGFR M1 H cells and human EGFR negative parental Renca cells, following transfection with EGFR-targeting polyplexes containing LPEI-l-[N₃:DBCO]PEG₃₆-hEGF and plasmid encoding hIL-2. Selective expression of hIL-2 from EGFR-overexpressing cells is demonstrated.

FIG. 15B depicts the levels of secreted human IL2 after transfection of low numbers of high EFGR expressing RencaEGFR M1 H cells (600 cells) with EGFR-targeting polyplexes containing LPEI-l-[N₃:DBCO]PEG₃₆-hEGF and plasmid encoding hIL2 at the indicated concentrations of the plasmid (0.125 and 0.25 μg/ml). The polyplexes were formulated at an N/P ratio of 6 and IL2 secretion was detected after 2, 3 and 4 days.

FIG. 16 depicts levels of secreted human IL2 normalized to cell survival, in cell lines with differential PSMA expression: high-expressing LNCaP and C4-2 cells, and low-expressing DU145 cells following transfection with PSMA-targeting polyplexes containing LPEI-l-[N₃:DBCO]PEG₃₆-DUPA and plasmid encoding IL2 protein. The X axis indicates the concentration of the hIL2 plasmid DNA (0.25, 0.5 and 1.0 μg/mL) in the polyplexes. The Y axis indicates the concentration of secreted IL2 normalized to cell survival in arbitrary units (AU). The selective expression/secretion of human IL2 after transfection of PSMA overexpressing cells with plasmid DNA encoding hIL-2 is demonstrated.

FIG. 17A depicts the level of secreted human IFNβ from RencaEGFR M1 H cancer cells, which have high human EGFRexpression, following transfection with EGFR-targeting polyplexes containing pCMV-hINFβ at N/P ratio N/P 3. The X axis indicates the concentration of pCMV-hIFNβ plasmid DNA (0.25, 0.5, 1.0, and 2.0 μg/mL) in the polyplexes. The Y axis indicates the concentration of secreted IFNβ protein in μg/mL and is presented as average with standard deviation from triplicate samples. Secretion of human IFNβ from EGFR overexpressing cancer cells is demonstrated for the tested delivery vectors, with a significant advantage of linear triconjugate vector LPEI-l-[N₃:DBCO]-PEG₃₆-hEGF over the random delivery vector.

FIG. 17B depicts the level of secreted human IFNβ from RencaEGFR M1 H cancer cells, which have high human EGFRexpression, following transfection with EGFR-targeting polyplexes containing pCMV-hINFβ at N/P ratio N/P 4. The X axis indicates the concentration of pCMV-hIFNβ plasmid DNA (0.25, 0.5, 1.0, and 2.0 μg/mL) in the polyplexes. The Y axis indicates the concentration of secreted IFNβ protein in pg/mL and is presented as average with standard deviation from triplicate samples. Secretion of human IFNβ from EGFR overexpressing cancer cells is demonstrated for the tested delivery vectors, with a significant advantage of linear triconjugate vector LPEI-l-[N₃:DBCO]-PEG₃₆-hEGF over the random delivery vector.

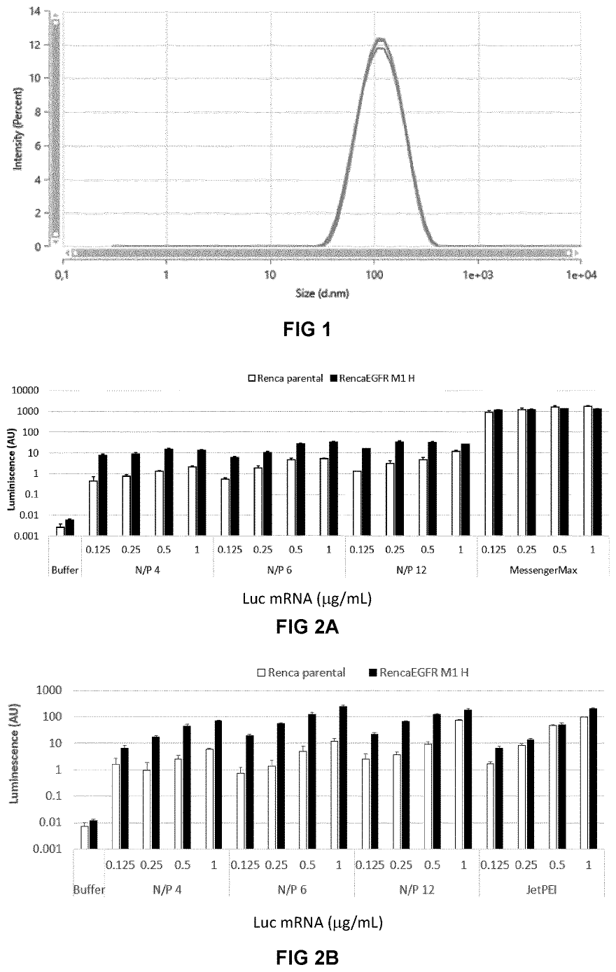

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether is specifically again referred to.

The present invention provides polyplexes of (i) nucleic acids that encode a peptide or protein of interest, preferably of pharmaceutically active nucleic acids encoding pharmaceutically active peptides or proteins such as cytokines, interferons, or toxins, and (ii) targeting linear conjugates of LPEI and PEG, as outlined herein and below. The conjugates preferably comprise an LPEI fragment, a PEG fragment, and a targeting fragment. In preferred embodiments, the LPEI fragment and the PEG fragment are coupled in a discrete end-to-end fashion. In some preferred embodiments, the LPEI fragment and the PEG fragment are coupled through the covalent attachment of an azide to an alkene or alkyne to form a 1,2,3-triazole or a 4,5-dihydro-1H-[1,2,3]triazole.

### Definitions

Unless defined otherwise, all technical and scientific terms used herein have the same meanings as commonly understood by one of ordinary skill in the art to which this disclosure belongs.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “about”, as used herein shall have the meaning of +/−10%. For example about 50% shall mean 45% to 55%. Preferably, the term “about”, as used herein shall have the meaning of +/−5%. For example about 50% shall mean 47.5% to 52.5%.

The phrase “between number X and number Y”, as used herein, shall refer to include the number X and the number Y. For example, the phrase “between 0.01 μmol and 50 μmol” refers to 0.01 μmol and 50 μmol and the values in between. The same applies to the phrase “between about number X and about number Y”.

The term “optionally substituted” is understood to mean that a given chemical moiety (e.g. an alkyl group) can (but is not required to) be bonded other substituents (e.g. heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, an alkoxy group, or any other substituent described herein. Thus the term “optionally substituted” means that a given chemical moiety has the potential to contain other functional groups, but does not necessarily have any further functional groups.

The term “optionally replaced” is understood to refer to situations in which the carbon atom of a methylene group (i.e., —CH₂—) can be, but is not required to be, replaced by a heteroatom (e.g., —NH—, —O—). For example, a C₃ alkylene (i.e., propylene) group wherein one of the methylene groups is “optionally replaced” can have the structure —CH₂—O—CH₂— or —O—CH₂—CH₂—. It will be understood by one of skill in the art that a methylene group cannot be replaced when such replacement would result in an unstable chemical moiety. For example, one of skill in the art will understand that four methylene groups cannot simultaneously be replaced by oxygen atoms. Thus, in some preferred embodiments, when one methylene group of an alkylene fragment is replaced by a heteroatom, one or both of the neighboring carbon atoms are not replaced by a heteroatom.

The term “aryl” refers to cyclic, aromatic hydrocarbon groups that have 1 to 2 aromatic rings, including monocyclic or bicyclic groups such as phenyl, biphenyl or naphthyl. A C₆-C₁₀ aryl group contains between 6 and 10 carbon atoms. When containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted. Furthermore, when containing two fused rings, the aryl groups herein defined may have an unsaturated or partially saturated ring fused with a fully saturated ring. Exemplary ring systems of these aryl groups include indanyl, indenyl, tetrahydronaphthalenyl, and tetrahydrobenzoannulenyl. In some preferred embodiments, the aryl group is a phenyl group.

Unless otherwise specifically defined, “heteroaryl” means a monovalent monocyclic aromatic ring of 5 to 24 ring atoms or a polycyclic aromatic ring, containing one or more ring heteroatoms selected from N, S, P, or O, the remaining ring atoms being C. A 5-10 membered heteroaryl group contains between 5 and 10 atoms. Heteroaryl as herein defined also means a bicyclic heteroaromatic group wherein the heteroatom is selected from N, S, P, or O. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ2-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two fused rings, the heteroaryl groups herein defined may have an unsaturated or partially saturated ring fused with a fully saturated ring. Exemplary ring systems of these heteroaryl groups include indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, indolinyl, indolyl, and dihydrobenzoxanyl.

The term “alkyl” refers to a straight or branched chain saturated hydrocarbon. C₁-C₆ alkyl groups contain 1 to 6 carbon atoms. Examples of a C₁-C₆ alkyl group include, but are not limited to, methyl, ethyl, propyl, butyl, pentyl, isopropyl, isobutyl, sec-butyl, tert-butyl, isopentyl and neopentyl.

The term “alkylene” refers to a straight or branched chain saturated and bivalent hydrocarbon fragment. C₀-C₆ alkyl groups contain 0 to 6 carbon atoms. Examples of a C₀-C₆ alkylene group include, but are not limited to, methylene, ethylene, propylene, butylene, pentylene, isopropylene, isobutylene, sec-butylene, tert-butylene, isopentylene, and neopentylene.

The term “C₁-C₆-alkoxy”, as used herein, refers to a substituted hydroxyl of the formula (—OR′), wherein R′ is an optionally substituted C₁-C₆ alkyl, as defined herein, and the oxygen moiety is directly attached to the parent molecule, and thus the term “C₁-C₆ alkoxy”, as used herein, refers to straight chain or branched C₁-C₆ alkoxy which may be, for example, methoxy, ethoxy, n-propoxy, isopropoxy, n-butoxy, isobutoxy, sec-butoxy, tert-butoxy, straight or branched pentoxy, straight or branched hexyloxy. Preferred are C₁-C₄ alkoxy and C₁-C₃ alkoxy.

The term “cycloalkyl” means monocyclic or polycyclic saturated carbon rings containing 3-18 carbon atoms. A C₃-C₈ cycloalkyl contains between 3 and 8 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl. A C₃-C₈ cycloalkyl is a cycloalkyl group containing between 3 and 8 carbon atoms.

The term “cycloalkenyl” means monocyclic, non-aromatic unsaturated carbon rings containing 5-18 carbon atoms. Examples of cycloalkenyl groups include, without limitation, cyclopentenyl, cyclohexenyl, cycloheptenyl, cyclooctenyl, and norborenyl. A C₅-C₈ cycloalkenyl is a cycloalkenyl group containing between 5 and 8 carbon atoms.

The terms “heterocyclyl” or “heterocycloalkyl” or “heterocycle” refer to monocyclic or polycyclic 3 to 24-membered rings containing carbon and heteroatoms taken from oxygen, nitrogen, or sulfur and wherein there is not delocalized π electrons (aromaticity) shared among the ring carbon or heteroatoms. A 3-10 membered heterocycloalkyl group contains between 3 and 10 atoms. Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl.

The term “heterocycloalkenyl” refers to monocyclic or polycyclic 3 to 24-membered rings containing carbon and heteroatoms taken from oxygen, nitrogen, or sulfur and wherein there is not delocalized π electrons (aromaticity) shared among the ring carbon or heteroatoms, but there is at least one element of unsaturation within the ring. A 3-10 membered heterocycloalkenyl group contains between 3 and 10 atoms.

As used herein, the term “halo” or “halogen” means fluoro (F), chloro (Cl), bromo (Br), or iodo (I).

The term “carbonyl” refers to a functional group composing a carbon atom double-bonded to an oxygen atom. It can be abbreviated herein as “oxo”, as C(O), or as C═O.

The term “polyplex” as used herein refers to a complex of a polymer and a nucleic acid typically and preferably formed via electrostatic interactions. In particular, the term “polyplex” as used herein refers to a complex of a conjugate as described herein for the present invention and a nucleic acid such as a single stranded RNA, preferably a mRNA, or a DNA, preferably a plasmid DNA. The term “polyplex” further typically and preferably refers to a vector, in particular a polymeric non-viral triconjugate vector as described herein for the present invention useful for carrying and delivering nucleic acids to the desired targeted cells.

The term “overexpression” refers to gene or protein expression within a cell or in a cell surface that is increased relative to basal or normal expression. In a preferred embodiment, said targeting fragment is capable of binding to a cell overexpressing a cell surface receptor. In one embodiment, said cell overexpressing a cell surface receptor means that the level of said cell surface receptor expressed in said cell of a certain tissue is elevated in comparison to the level of said cell surface receptor as measured in a normal healthy cell of the same type of tissue under analogous conditions. In one embodiment, said cell overexpressing a cell surface receptor refers to an increase in the level of said cell surface receptor in a cell relative to the level in the same cell or closely related non-malignant cell under normal physiological conditions.

The term “polyanion”, as used herein, refers to a polymer, preferably a biopolymer, having more than one site carrying a negative charge. Typically and preferably, the term “polyanion”, as used herein, refers to a polymer, preferably a biopolymer, made up of repeating units comprising residues capable of bearing negative charge. In further embodiments, a polyanion is a polymer, preferably a biopolymer, made up of repeating units comprising negatively charged residues. In another preferred embodiment, said polyanion is a nucleic acid, more preferably a DNA, RNA, polyglutamic acid or hyaluronic acid.

The term “nucleic acid” as used herein, comprises deoxyribonucleic acid (DNA) and/or ribonucleic acid (RNA) or a combination thereof. In a preferred embodiment, the term “nucleic acid” refers to deoxyribonucleic acid (DNA) and/or ribonucleic acid (RNA), and hereby to genomic, viral and recombinantly prepared and chemically synthesized molecules. A nucleic acid may be in the form of a single stranded or double-stranded and linear or covalently closed circular molecule and may comprise a chemical derivatization of a nucleic acid on a nucleotide base, on the sugar or on the phosphate, and may contain non-natural nucleotides and nucleotide analogs.

The term “dispersity” (abbreviated as D), as used herein refers to the distribution of the molar mass in a given polymeric sample such as in polymeric fragments as used herein for the inventive conjugates and polyplexes. It is defined herein as D=(M_{w}/Mₙ), wherein D is dispersity; M_{w} is the weight average molecular weight of the polymeric sample or polymeric fragment; and Mₙ is the number average molecular weight of the polymeric sample or polymeric fragment.

The term “weight average molecular weight”, as used herein refers to the sum of the products of the weight fraction for a given molecule in the mixture times the mass of the molecule for each molecule in the mixture and is typically and preferably represented by the symbol Mw.

The term “number average molecular weight”, as used herein refers to the total weight of a mixture divided by the number of molecules in the mixture and is typically and preferably represented by the symbol Mn.

The term “polydispersity index” (abbreviated as PDI) as used herein refers to the polydispersity index in dynamic light scattering measurements of polyplex nanoparticles such as the polyplexes in accordance with the present invention. This index is a number calculated from a simple 2 parameter fit to the correlation data (the cumulants analysis). The polydispersity index is dimensionless and scaled such that values smaller than 0.05 are rarely seen other than with highly monodisperse standards. Values greater than 0.7 indicate that the sample has a very broad size distribution and is probably not suitable for the dynamic light scattering (DLS) technique. The various size distribution algorithms work with data that falls between these two extremes. The zeta-average diameter (z-average diameter) and polydispersity index of the inventive polyplexes are determined by Dynamic Light Scattering (DLS), based on the assumption that said polyplexes are isotropic and spherically shaped. The calculations for these parameters are defined and determined according to ISO standard document ISO 22412:2017.

The term “amino acid residue” refers to a divalent residue derived from an organic compound containing the functional groups amine (—NH₂) and carboxylic acid (—COOH), typically and preferably, along with a side chain specific to each amino acid. In a preferred embodiment of the present invention, an amino acid residue is divalent residue derived from an organic compound containing the functional groups amine (—NH₂) and carboxylic acid (—COOH), wherein said divalence is effected with said amine and said carboxylic acid functional group, and thus by —NH— and —CO—moieties. In alternative preferred embodiment of the present invention, an amino acid residue is a divalent residue derived from an organic compound containing the functional groups amine (—NH₂) and carboxylic acid (—COOH), wherein said divalence is effected with said amine or said carboxylic acid functional group, and with a further functional group present in said amino acid residue. By way of a preferred example and embodiment, an amino acid residue in accordance with the present invention derived from cysteine includes the divalent structure —S—(CH2)—CH(COOH)—NH—, wherein said divalence is effected by the amino functionality and the comprised thiol functionality. The term “amino acid residue”, as used herein typically and preferably includes amino acid residues derived from naturally occurring or non-naturally occurring amino acids. Furthermore, the term “amino acid residue”, as used herein, typically and preferably also includes amino acid residues derived from unnatural amino acids that are chemically synthesized including alpha-(α-), beta-(β-), gamma-(γ-) or delta-(δ-) etc. amino acids as well as mixtures thereof in any ratio. In addition, the term “amino acid residue”, as used herein, typically and preferably also includes amino acid residues derived from alpha amino acids including any isomeric form thereof, in particular its D-stereoisomers and L-stereoisomers (alternatively addressed by the (R) and (S) nomenclature), as well as mixtures thereof in any ratio, preferably in a racemic ratio of 1:1. The term “D-stereoisomer”, “L-stereoisomer”, “D-amino acid” or “L-amino acid” refers to the chiral alpha carbon of the amino acids. Thus, in a preferred embodiment, said amino acid residue is a divalent group of the structure —NH—CHR—C(O)—, wherein R is an amino acid side chain. Two or more consecutive amino acid residues preferably form peptide (i.e., amide) bonds at both the amine portion and the carboxylic acid portion of the amino acid residues respectively. When di, tri or polypeptides are described herein as amino acid residues, typically as (AA)ₐ, the provided sequence is depicted from left to right in the N-C direction. Thus, and by way of example the (AA)ₐ being Trp-Trp-Gly should refer to an amino acid residue, wherein Trp corresponds to the N-terminus of said tripeptide with a —NH—valence, and wherein Gly corresponds to the C-terminus of said tripeptide with a —CO—valence.

The terms “peptide”, “polypeptide” and “protein”, as used herein refers to substances which comprise about two or more consecutive amino acid residues linked to one another via peptide bonds. The terms “peptide,” “polypeptide,” and “protein” are used interchangeably herein to refer to polymers of amino acid residues of any length. In one embodiment, the term “protein” refers to large peptides, in particular peptides having at least about 151 amino acids, while in one embodiment, the term “peptide” refers to substances which comprise about two or more, about 3 or more, about 8 or more, or about 20 or more, and up to about 50, about 100 or about 150.

The term “disease-associated antigen”, as used herein, refers in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with infection by microbes, typically microbial antigens, or associated with cancer, typically tumors.

The term “viral antigen”, as used herein, refers to any viral component having antigenic properties, i.e. being able to provoke an immune response in an individual. The viral antigen may be a viral ribonucleoprotein or an envelope protein.

The term “bacterial antigen”, as used herein, refers to any bacterial component having antigenic properties, i.e. being able to provoke an immune response in an individual. The bacterial antigen may be derived from the cell wall or cytoplasm membrane of the bacterium.

The term “epitope”, as used herein, refers to a part or fragment of a molecule such as an antigen that is recognized by the immune system. For example, the epitope may be recognized by T cells, B cells or antibodies. An epitope of an antigen preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes. The term “T cell epitope”, as used herein, refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell.

The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE. As used herein, an antibody fragment (i.e. characteristic portion of an antibody) refers to any derivative of an antibody which is less than full-length. In general, an antibody fragment retains at least a significant portion of the full-length antibody's specific binding ability. Examples of antibody fragments include, but are not limited to, single chain and double strain fragments, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids. In some embodiments, antibodies may include chimeric (e.g. “humanized”) and single chain (recombinant) antibodies. In some embodiments, antibodies may have reduced effector functions and/or bispecific molecules. In some embodiments, antibodies may include fragments produced by a Fab expression library. Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility. Diabodies are dimeric scFvs. Diabodies typically have shorter peptide linkers than most scFvs, and they often show a preference for associating as dimers. An Fv fragment is an antibody fragment which consists of one VH and one VL domain held together by noncovalent interactions. The term “dsFv” as used herein refers to an Fv with an engineered intermolecular disulfide bond to stabilize the VH-VL pair. A F(ab′)2 fragment is an antibody fragment essentially equivalent to that obtained from immunoglobulins by digestion with an enzyme pepsin at pH 4.0-4.5. The fragment may be recombinantly produced. A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced. 1. A Fab fragment is an antibody fragment essentially equivalent to that obtained by digestion of immunoglobulins with an enzyme (e.g. papain). The Fab fragment may be recombinantly produced. The heavy chain segment of the Fab fragment is the Fd sub-fragment.

The term “alpha terminus of the linear polyethyleneimine fragment” (α-terminus of LPEI fragment), as used herein, refers to the terminal end of the LPEI fragment where initiation of polymerization occurs using electrophilic initiators as further described below for the term “initiation residue”.

The term “omega terminus of the linear polyethyleneimine fragment” (ω-terminus of LPEI fragment) as used herein, refers to the terminal end of the LPEI fragment where termination of polymerization occurs using nucleophiles such as azides, thiol and other nucleophiles as described herein.

The term “organic residue” refers to any suitable organic group capable of binding to the nitrogen atoms embedded within LPEI fragments. In preferred embodiments the organic residue is connected to the nitrogen atom via a carbonyl group to form an amide linkage. Without wishing to be bound by theory, said organic residue is incorporated on the nitrogen atoms of poly(2-oxazoline) during ring-opening polymerization 2-oxazoline (see, e.g., Glassner et al., (2018), Poly(2-oxazoline)s: A comprehensive overview of polymer structures and their physical properties. Polym. Int, 67: 32-45. https://doi.org/10.1002/pi.5457). Typically and preferably, said organic residue is cleaved (i.e., typically said amide is cleaved) from the poly(2-oxazoline) to yield LPEI and LPEI fragments and thus —(NH—CH₂—CH₂)-moieties embedded within the conjugates of the present invention. However, in case said cleavage reaction is not complete a fraction of said organic residue is not cleaved. Thus, in preferred embodiments of the invention at least 80%, preferably 90% of R² in the R¹—(NR²—CH₂—CH₂)ₙ-moieties of the conjugates of the present invention including the ones of Formula I* and I is H, preferably at least 91%, more preferably 92%, more preferably 93%, more preferably 94%, more preferably 95%, more preferably 96%, more preferably 97%, more preferably 98%, and most preferably 99%, of R² in the R¹—(NR²—CH₂—CH₂)ₙ-moieties of the conjugates of the present invention including the ones of Formula I* or I is H.

The term “initiation residue” refers to the residue present in the LPEI fragment and the R¹—(NR²—CH₂—CH₂),-moieties of the conjugates of the present invention, which residue derives from any initiator, typically and preferably any electrophilic initiator, capable of initiating the polymerization of poly(2-oxazoline) from 2-oxazoline. As set forth in Glassner et al., (2018), Poly(2-oxazoline)s: A comprehensive overview of polymer structures and their physical properties. Polym. Int, 67: 32-45. https://doi.org/10.1002/pi.5457, “different initiator systems can be used including toluenesulfonic acid (TsOH) or alkyl sulfonates such as methyl p-toluenesulfonate (MeOTs), which is most frequently found in literature, p-nitrobenzenesulfonates (nosylates) and trifluoromethanesulfonates (triflates), alkyl, benzyl and acetyl halides, oxazolinium salts and lewis acids.” Accordingly, although in preferred embodiments R¹ is —H or —CH₃, one of skill in the art will understand that R¹ can also include but is not limited to other suitable residues such as a Cn alkyl group wherein n is greater than 1, typically a C₁₋₆ alkyl group, a benzyl group, or an acetyl group.

The present invention provides targeting polyplexes comprised of (i) nucleic acids, in particular, nucleic acids encoding pharmaceutically active peptides or proteins such as cytokines, interferons, or toxins, and (ii) targeting conjugates comprising LPEI and PEG fragments that are connected by discrete linkages formed through defined, chemoselective reactions instead of through random and uncontrolled bonding of an electrophilic PEG fragment to multiple nucleophiles of an LPEI backbone fragment. The discrete linkages not only ensure consistent and predictable ratios of LPEI to PEG fragments, but further ensure defined linear instead of random branched conjugates. Thus, the LPEI fragment is bonded in a linear end-to-end fashion to a single PEG fragment. The chemoselective bonding of the LPEI fragments to the PEG fragments can take place using any suitable chemical precursors that can form a chemoselective bond. In preferred embodiments, the chemoselective bonding of LPEI fragments to PEG fragments takes place by means of a [3+2]cycloaddition between an azide and an alkyne or alkene. Alternatively, said chemoselective bonding is by means of a thiol-ene reaction between a thiol and an alkene. When the chemoselective bond is between an azide and an alkyne or alkene, the resulting linkage is a 1,2,3-triazole (when an alkyne is coupled) or a 4,5-dihydro-1H-[1,2,3]triazole (when an alkene is coupled). When the chemoselective bond is between a thiol and an alkene, the resulting linkage is a thioether.

The conjugates further comprise targeting fragments linked to the PEG fragments which allow to target a particular cell type and to facilitate the uptake of the inventive compositions and pharmaceutically active nucleic acids in said particular cell type. Thus, preferred embodiments comprise targeting fragments such as hEGF, DUPA or folate specifically connected to the LPEI-PEG diconjugates to target the corresponding receptors such hEGFR, PSMA or folate receptor on the particular cell types, typically cancer cell types, on which said receptors show high expression and are overexpressed.

Further advantageously and surprisingly, the inventors have found that the resulting preferred conjugates and polyplexes in accordance with the present invention which have a significant reduced heterogeneity due to the defined chemoselective bonding of the LPEI fragments to the PEG fragments, and thus, which have a significant reduced number of potentially biologically active conjugates and polyplexes, not only form polyplexes of suitable size, but also maintain or even increase their overall biological activity such as highly selective targeted delivery of the pharmaceutically active nucleic acids as well as the subsequent efficient translation and secretion of the encoded pharmaceutically active proteins. Thus, the inventive compositions and polyplexes do not only selectively deliver pharmaceutically active nucleic acids encoding pharmaceutically active peptides or proteins to the targeted cells, in particular cancer cells, but furthermore, said delivery results in high expression and efficient protein translation as well as secretion of the encoded pharmaceutically active proteins.

Thus, in one aspect, the present invention provides a composition comprising a polyplex, wherein said polyplex comprise a conjugate and a nucleic acid, wherein said nucleic acid is preferably non-covalently bound to said conjugate, and wherein said conjugate comprises: a linear polyethyleneimine fragment comprising an alpha terminus and an omega terminus; wherein the alpha terminus of said polyethyleneimine fragment is an initiation residue; a polyethylene glycol fragment comprising a first terminal end and a second terminal end; and wherein the omega terminus of the polyethyleneimine fragment is connected to the first terminal end of the polyethylene glycol fragment by a divalent covalent linking group —Z—X¹—, wherein —Z—X¹ is not a single bond and —Z— is not an amide; wherein the second terminal end of the polyethylene glycol fragment is connected to a targeting fragment by a divalent covalent linking moiety X², and wherein said nucleic acid is a nucleic acid that encodes a peptide or protein of interest, wherein preferably said nucleic acid is a pharmaceutically active nucleic acid, and wherein said pharmaceutically active nucleic acid is a nucleic acid that encodes a pharmaceutically active peptide or protein. In a preferred embodiment of this aspect, said composition consists of said polyplex. In a preferred embodiment, linear polyethyleneimine fragment is of the formula R¹—(NR²—CH₂—CH₂)ₙ—, n is any integer between 1 and 1500. In a further preferred embodiment, said R¹—(NR²—CH₂—CH₂)ₙ-moiety is a disperse polymeric moiety with between about 115 and about 1150 repeating units n and a dispersity of about 5 or less, preferably between about 280 and about 700 repeating units n with a dispersity of about 3 or less, and further preferably between about 350 and about 630 repeating units n with a dispersity of about 2 or less, and wherein preferably R¹ is —H or —CH₃.

In another aspect, the present invention provides a composition comprising a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

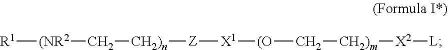

wherein n is any integer between 1 and 1500, preferably any integer between 2 and 1500; m is any integer between 1 and 200, preferably m is any integer between 2 and 200, preferably any integer between 1 and 100, and further preferably any integer between 2 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ—is H; X¹ and X² are independently divalent covalent linking moieties; Z is a divalent covalent linking moiety wherein Z is not a single bond and Z is not —NHC(O)—; L is a targeting fragment, wherein preferably said targeting fragment is capable of binding to a cell, and wherein further preferably said targeting fragment is capable of binding to a cell surface receptor, and wherein said nucleic acid is a nucleic acid that encodes a peptide or protein of interest, wherein preferably said nucleic acid is a pharmaceutically active nucleic acid, and wherein said pharmaceutically active nucleic acid is a nucleic acid that encodes a pharmaceutically active peptide or protein. Preferably said composition consists of said polyplex.

In another aspect, the present invention provides a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

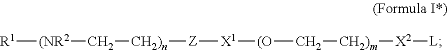

wherein n is any integer between 1 and 1500, preferably any integer between 2 and 1500; m is any integer between 1 and 200, preferably m is any integer between 2 and 200, preferably any integer between 1 and 100, and further preferably any integer between 2 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ—is H; X¹ and X² are independently divalent covalent linking moieties; Z is a divalent covalent linking moiety wherein Z is not a single bond and Z is not —NHC(O)—; L is a targeting fragment, wherein preferably said targeting fragment is capable of binding to a cell, and wherein further preferably said targeting fragment is capable of binding to a cell surface receptor, wherein said nucleic acid is a nucleic acid that encodes a peptide or protein of interest, wherein preferably said nucleic acid is a pharmaceutically active nucleic acid, and wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

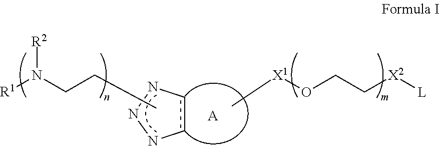

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a nucleic acid that encodes a peptide or protein of interest, wherein preferably said nucleic acid is a pharmaceutically active nucleic acid, and wherein said pharmaceutically active nucleic acid is a nucleic acid that encodes a pharmaceutically active peptide or protein.

As noted herein, the depiction of Formula I above represents two different regioisomeric attachments of the fragment R¹(NR²CH₂CH₂)ₙ, i.e.,

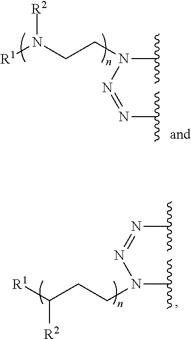

wherein the wavy lines represent chemical bonds to Ring A. Accordingly, Formula I as drawn herein encompasses two regioisomeric embodiments, i.e., wherein the fragment R¹(NR²CH₂CH₂)ₙ is bonded at the top nitrogen atom in the structures above or at the bottom nitrogen atom in the structures above, but not at the middle nitrogen atom. Formula I as drawn above is used interchanageably herein with the equivalent depiction of Formula I comprising a fragment H—N—N═N below, i.e.,

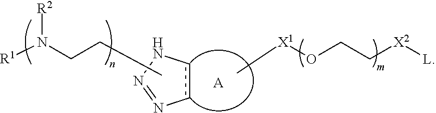

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

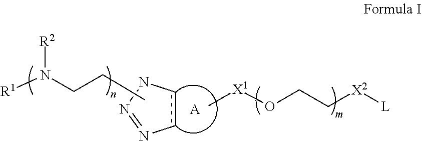

wherein:
[symbol] is a single bond or a double bond;
n is any integer between 1 and 1500, preferably any integer between 2 and 1500;
m is any integer between 2 and 200, preferably any integer between 1 and 200, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a nucleic acid that encodes a peptide or protein of interest, wherein preferably said nucleic acid is a pharmaceutically active nucleic acid, and wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

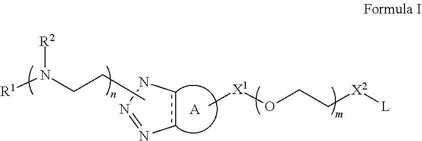

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ-moieties is H;
Ring A is a 5 to 10-membered cycloalkyl, cycloalkenyl, heterocycloalkyl, or heterocycloalkenyl, optionally substituted at any position with one or more R^{A1};
R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2};
R^{A2} is independently selected from C₁-C₆ alkyl, C₁-C₆ alkoxy, halogen —SO₃H, or —OSO₃H;
X¹ is a linking moiety of the formula —(Y¹)ₚ—, wherein p is an integer between 1 and 20, and each occurrence of Y¹ is independently selected from a chemical bond, —CR¹¹R¹²—, —C(O)—, —O—, —S—, —NR¹³—, an amino acid residue, a divalent phenyl moiety, a divalent heterocycle moiety, and a divalent heteroaryl moiety, wherein each divalent phenyl or heteroaryl is optionally substituted with one or more R¹³, and each divalent heterocycle is optionally substituted with one or more R¹⁴; wherein R¹¹, R¹² and R¹³ are independently, at each occurrence, H or C₁-C₆ alkyl; and wherein R¹⁴ is independently, at each occurrence, H, C₁-C₆ alkyl, or oxo;
X² is a linking moiety of the formula —(Y²)_{q}—, wherein q is an integer between 1 and 50, and each occurrence of Y² is independently selected from a chemical bond, —CR²¹R²²—, NR²³—, —O—, —S—, —C(O)—, an amino acid residue, a divalent phenyl moiety, a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴; wherein R²¹, R²², and R²³ are each independently, at each occurrence, —H, —CO₂H, or C₁-C₆ alkyl, wherein each C₁-C₆ alkyl is optionally substituted with one or more —OH, oxo, C₆-C₁₀ aryl, or 5 to 8-membered heteroaryl; and wherein R²⁴ is independently, at each occurrence, —H, —CO₂H, C₁-C₆ alkyl, or oxo; and
L is a targeting fragment preferably capable of binding to a cell, and wherein preferably said targeting fragment is capable of binding to a cell surface receptor, and
wherein said nucleic acid is a nucleic acid that encodes a peptide or protein of interest, wherein preferably said nucleic acid is a pharmaceutically active nucleic acid, and wherein said pharmaceutically active nucleic acid is a nucleic acid that encodes a pharmaceutically active peptide or protein. Preferably said composition consists of said polyplex.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

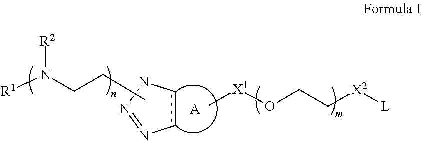

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ-moieties is H;
Ring A is a 5 to 10-membered cycloalkyl, cycloalkenyl, heterocycloalkyl, or heterocycloalkenyl, optionally substituted at any position with one or more R^{A1};
R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2};
R^{A2} is independently selected from C₁-C₆ alkyl, C₁-C₆ alkoxy, halogen —SO₃H, or —OSO₃H;
X¹ is a linking moiety of the formula —(Y¹)ₚ—, wherein p is an integer between 1 and 20, and each occurrence of Y¹ is independently selected from a chemical bond, —CR¹¹R¹²—, —C(O)—, —O—, —S—, —NR¹³—, an amino acid residue, a divalent phenyl moiety, a divalent heterocycle moiety, and a divalent heteroaryl moiety, wherein each divalent phenyl or heteroaryl is optionally substituted with one or more R¹³, and each divalent heterocycle is optionally substituted with one or more R¹⁴; wherein R¹¹, R¹² and R¹³ are independently, at each occurrence, H or C₁-C₆ alkyl; and wherein R¹⁴ is independently, at each occurrence, H, C₁-C₆ alkyl, or oxo;
X² is a linking moiety of the formula —(Y²)_{q}—, wherein q is an integer between 1 and 50, and each occurrence of Y² is independently selected from a chemical bond, —CR²¹R²²—, NR²³—, —O—, —S—, —C(O)—, an amino acid residue, a divalent phenyl moiety, a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴; wherein R²¹, R²², and R²³ are each independently, at each occurrence, —H, —CO₂H, or C₁-C₆ alkyl, wherein each C₁-C₆ alkyl is optionally substituted with one or more —OH, oxo, C₆-C₁₀ aryl, or 5 to 8-membered heteroaryl; and wherein R²⁴ is independently, at each occurrence, —H, —CO₂H, C₁-C₆ alkyl, or oxo; and
L is a targeting fragment preferably capable of binding to a cell and wherein preferably said targeting fragment is capable of binding to a cell surface receptor, and
wherein said nucleic acid is a nucleic acid that encodes a peptide or protein of interest, wherein preferably said nucleic acid is a pharmaceutically active nucleic acid, and wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

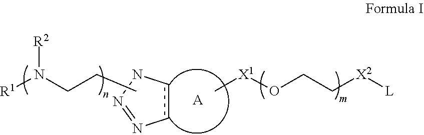

wherein:
[symbol] is a single bond or a double bond;
n is any integer between 1 and 1500, preferably any integer between 2 and 1500;
m is any discrete number of repeating —(O—CH₂—CH₂)—units of 25 to 100, preferably of 25 to 60, wherein preferably said discrete number m is a discrete number of contiguous repeating —(O—CH₂—CH₂)—units, and wherein said discrete number of contiguous repeating —(O—CH₂—CH₂)—units) is any discrete number of 25 to 100, preferably of 25 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

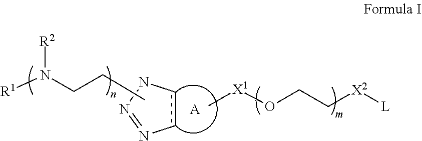

wherein:
[symbol] is a single bond or a double bond;
n is any integer between 1 and 1500, preferably any integer between 2 and 1500;
m is any discrete number of repeating —(O—CH₂—CH₂)—units of 25 to 100, preferably of 25 to 60, wherein preferably said discrete number m is a discrete number of contiguous repeating —(O—CH₂—CH₂)—units, and wherein said discrete number of contiguous repeating —(O—CH₂—CH₂)—units) is any discrete number of 25 to 100, preferably of 25 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

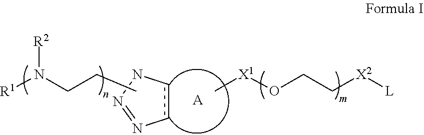

wherein:
[symbol] is a single bond or a double bond;
n is any integer between 1 and 1500, preferably any integer between 2 and 1500;
m is a discrete number of repeating —(O—CH₂—CH₂)—units of 36, wherein preferably said discrete number m is a discrete number of contiguous repeating —(O—CH₂—CH₂)—units, and wherein said discrete number of contiguous repeating —(O—CH₂—CH₂)—units) is 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

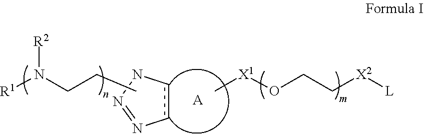

wherein:
[symbol] is a single bond or a double bond;
n is any integer between 1 and 1500, preferably any integer between 2 and 1500;
m is a discrete number of repeating —(O—CH₂—CH₂)—units of 36, wherein preferably said discrete number m is a discrete number of contiguous repeating —(O—CH₂—CH₂)—units, and wherein said discrete number of contiguous repeating —(O—CH₂—CH₂)—units) is 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising polyplexes, wherein each of said polyplex comprise a conjugate and a nucleic acid, wherein said nucleic acid is preferably non-covalently bound to said conjugate, and wherein said conjugate comprises: a linear polyethyleneimine fragment comprising an alpha terminus and an omega terminus; wherein the alpha terminus of said polyethyleneimine fragment is an initiation residue; a polyethylene glycol fragment comprising a first terminal end and a second terminal end; and wherein the omega terminus of the polyethyleneimine fragment is connected to the first terminal end of the polyethylene glycol fragment by a divalent covalent linking group —Z—X¹—, wherein —Z—X¹ is not a single bond and —Z— is not an amide; wherein the second terminal end of the polyethylene glycol fragment is connected to a targeting fragment by a divalent covalent linking moiety X², and wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate and a nucleic acid, wherein said nucleic acid is preferably non-covalently bound to said conjugate, and wherein said conjugate comprises: a linear polyethyleneimine fragment comprising an alpha terminus and an omega terminus; wherein the alpha terminus of said polyethyleneimine fragment is an initiation residue; a polyethylene glycol fragment comprising a first terminal end and a second terminal end; and wherein the omega terminus of the polyethyleneimine fragment is connected to the first terminal end of the polyethylene glycol fragment by a divalent covalent linking group —Z—X¹—, wherein —Z—X¹ is not a single bond and —Z— is not an amide; wherein the second terminal end of the polyethylene glycol fragment is connected to a targeting fragment by a divalent covalent linking moiety X², and wherein said nucleic acid is a RNA, wherein said RNA is a ssRNA, and wherein preferably said ssRNA is a mRNA. In a preferred embodiment of this aspect, said composition consists of said polyplex.

In another aspect, the present invention provides a composition comprising a polyplex, wherein said polyplex comprise a conjugate and a nucleic acid, wherein said nucleic acid is preferably non-covalently bound to said conjugate, and wherein said conjugate comprises: a linear polyethyleneimine fragment comprising an alpha terminus and an omega terminus; wherein the alpha terminus of said polyethyleneimine fragment is an initiation residue; a polyethylene glycol fragment comprising a first terminal end and a second terminal end; and wherein the omega terminus of the polyethyleneimine fragment is connected to the first terminal end of the polyethylene glycol fragment by a divalent covalent linking group —Z—X¹—, wherein —Z—X¹ is not a single bond and —Z— is not an amide; wherein the second terminal end of the polyethylene glycol fragment is connected to a targeting fragment by a divalent covalent linking moiety X², and wherein said nucleic acid is a RNA, wherein said RNA is a ssRNA, and wherein preferably said ssRNA is a mRNA, and wherein further preferably said mRNA encodes a peptide or protein of interest. In a preferred embodiment of this aspect, said composition consists of said polyplex.

In another aspect, the present invention provides a composition comprising polyplexes, wherein each of said polyplex comprise a conjugate and a nucleic acid, wherein said nucleic acid is preferably non-covalently bound to said conjugate, and wherein said conjugate comprises: a linear polyethyleneimine fragment comprising an alpha terminus and an omega terminus; wherein the alpha terminus of said polyethyleneimine fragment is an initiation residue; a polyethylene glycol fragment comprising a first terminal end and a second terminal end; and wherein the omega terminus of the polyethyleneimine fragment is connected to the first terminal end of the polyethylene glycol fragment by a divalent covalent linking group —Z—X¹—, wherein —Z—X¹ is not a single bond and —Z— is not an amide; wherein the second terminal end of the polyethylene glycol fragment is connected to a targeting fragment by a divalent covalent linking moiety X², and wherein said nucleic acid is a RNA, wherein said RNA is a ssRNA, and wherein preferably said ssRNA is a mRNA.

In another aspect, the present invention provides a composition comprising polyplexes, wherein each of said polyplex comprise a conjugate and a nucleic acid, wherein said nucleic acid is preferably non-covalently bound to said conjugate, and wherein said conjugate comprises: a linear polyethyleneimine fragment comprising an alpha terminus and an omega terminus; wherein the alpha terminus of said polyethyleneimine fragment is an initiation residue; a polyethylene glycol fragment comprising a first terminal end and a second terminal end; and wherein the omega terminus of the polyethyleneimine fragment is connected to the first terminal end of the polyethylene glycol fragment by a divalent covalent linking group —Z—X¹—, wherein —Z—X¹ is not a single bond and —Z— is not an amide; wherein the second terminal end of the polyethylene glycol fragment is connected to a targeting fragment by a divalent covalent linking moiety X², and wherein said nucleic acid is a DNA, wherein said DNA is a pDNA, and wherein preferably said pDNA encodes a peptide or protein of interest.

In another aspect, the present invention provides a composition comprising polyplexes, wherein each of said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

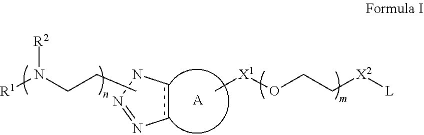

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

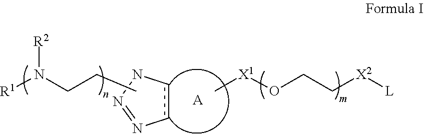

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a RNA, wherein said RNA is a ssRNA, and wherein preferably said ssRNA is a mRNA, and wherein further preferably said mRNA encodes a peptide or protein of interest.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

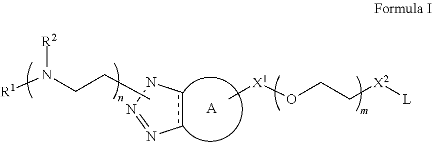

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a DNA, wherein said DNA is a pDNA, and wherein preferably said pDNA encodes a peptide or protein of interest.

In another aspect, the present invention provides a composition comprising polyplexes, wherein each said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

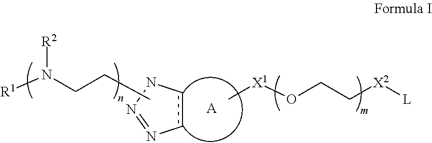

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a RNA, wherein said RNA is a ssRNA, and wherein preferably said ssRNA is a mRNA, and wherein further preferably said mRNA encodes a peptide or protein of interest.

In another aspect, the present invention provides a composition comprising polyplexes, wherein each said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

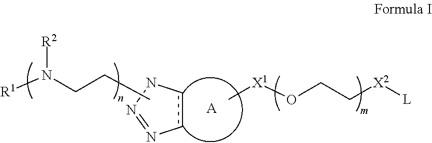

wherein:
[symbol] is a single bond or a double bond;
n is any integer between 1 and 1500, preferably any integer between 2 and 1500;
m is any integer between 1 and 200, preferably m is any integer between 2 and 200, preferably any integer between 1 and 100, and further preferably any integer between 2 and 100;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a DNA, wherein said DNA is a pDNA, and wherein preferably said pDNA encodes a peptide or protein of interest.

In another aspect, the present invention provides a composition comprising a polyplex, wherein said polyplex comprise a conjugate and a nucleic acid, wherein said nucleic acid is preferably non-covalently bound to said conjugate, and wherein said conjugate comprises a linear polyethyleneimine (LPEI) fragment covalently linked to one or more polyethylene glycol (PEG) fragments, each PEG fragment being covalently linked to a targeting fragment L, wherein preferably said targeting fragment is capable of binding to a cell; and wherein said nucleic acid is a single stranded RNA (ssRNA), wherein preferably said ssRNA is a mRNA, and wherein further preferably said mRNA is a pharmaceutically active nucleic acid, wherein said pharmaceutically active nucleic acid is a nucleic acid that encodes a pharmaceutically active peptide or protein, wherein again further preferably said pharmaceutically active peptide or protein is selected from a cytokine, an interferon, an interleukine, a growth factor, a hormone, an enzyme, a toxin, a tumor antigen, a viral antigen, bacterial antigen, an autoantigen, and an allergen.

In another aspect, the present invention provides a polyplex, wherein said polyplex comprise a conjugate and a nucleic acid, wherein said nucleic acid is preferably non-covalently bound to said conjugate, and wherein said conjugate comprises a linear polyethyleneimine (LPEI) fragment covalently linked to one or more polyethylene glycol (PEG) fragments, each PEG fragment being covalently linked to a targeting fragment L, wherein preferably said targeting fragment is capable of binding to a cell; and wherein said nucleic acid is a single stranded RNA (ssRNA), wherein preferably said ssRNA is a mRNA, and wherein further preferably said mRNA is a pharmaceutically active nucleic acid, wherein said pharmaceutically active nucleic acid is a nucleic acid that encodes a pharmaceutically active peptide or protein, wherein again further preferably said pharmaceutically active peptide or protein is selected from a cytokine, an interferon, an interleukine, a growth factor, a hormone, an enzyme, a toxin, a tumor antigen, a viral antigen, bacterial antigen, an autoantigen, and an allergen.

In another aspect, the present invention provides a composition comprising polyplexes, wherein each of said polyplex comprise a conjugate and a nucleic acid, wherein said nucleic acid is preferably non-covalently bound to said conjugate, and wherein said conjugate comprises a linear polyethyleneimine (LPEI) fragment covalently linked to one or more polyethylene glycol (PEG) fragments, each PEG fragment being covalently linked to a targeting fragment L, wherein preferably said targeting fragment is capable of binding to a cell; and wherein said nucleic acid is a single stranded RNA (ssRNA), wherein preferably said ssRNA is a mRNA, and wherein further preferably said mRNA is a pharmaceutically active nucleic acid, wherein said pharmaceutically active nucleic acid is a nucleic acid that encodes a pharmaceutically active peptide or protein, wherein again further preferably said pharmaceutically active peptide or protein is selected from a cytokine, an interferon, an interleukine, a growth factor, a hormone, an enzyme, a toxin, a tumor antigen, a viral antigen, bacterial antigen, an autoantigen, and an allergen.

In another aspect, the present invention provides a composition comprising a polyplex, wherein said polyplex comprise a conjugate and a nucleic acid, wherein said nucleic acid is preferably non-covalently bound to said conjugate, and wherein said conjugate comprises a linear polyethyleneimine (LPEI) fragment covalently linked to one or more polyethylene glycol (PEG) fragments, each PEG fragment being covalently linked to a targeting fragment L, wherein preferably said targeting fragment is capable of binding to a cell; and wherein said nucleic acid is a DNA, preferably a plasmid DNA (pDNA), wherein preferably said DNA, preferably said pDNA, encodes a pharmaceutically active peptide or protein, wherein said pharmaceutically active peptide or protein is preferably selected from a cytokine, an interferon, an interleukine, a growth factor, a hormone, an enzyme, a toxin, a tumor antigen, a viral antigen, bacterial antigen, an autoantigen, and an allergen.

In another aspect, the present invention provides a polyplex, wherein said polyplex comprise a conjugate and a nucleic acid, wherein said nucleic acid is preferably non-covalently bound to said conjugate, and wherein said conjugate comprises a linear polyethyleneimine (LPEI) fragment covalently linked to one or more polyethylene glycol (PEG) fragments, each PEG fragment being covalently linked to a targeting fragment L, wherein preferably said targeting fragment is capable of binding to a cell; and wherein said nucleic acid is a DNA, preferably a plasmid DNA (pDNA), wherein preferably said DNA, preferably said pDNA, encodes a pharmaceutically active peptide or protein, wherein said pharmaceutically active peptide or protein is preferably selected from a cytokine, an interferon, an interleukine, a growth factor, a hormone, an enzyme, a toxin, a tumor antigen, a viral antigen, bacterial antigen, an autoantigen, and an allergen.

In another aspect, the present invention provides a composition comprising polyplexes, wherein each of said polyplex comprise a conjugate and a nucleic acid, wherein said nucleic acid is preferably non-covalently bound to said conjugate, and wherein said conjugate comprises a linear polyethyleneimine (LPEI) fragment covalently linked to one or more polyethylene glycol (PEG) fragments, each PEG fragment being covalently linked to a targeting fragment L, wherein preferably said targeting fragment is capable of binding to a cell; and wherein said nucleic acid is a DNA, preferably a plasmid DNA (pDNA), wherein preferably said DNA, preferably said pDNA, encodes a pharmaceutically active peptide or protein, wherein said pharmaceutically active peptide or protein is preferably selected from a cytokine, an interferon, an interleukine, a growth factor, a hormone, an enzyme, a toxin, a tumor antigen, a viral antigen, bacterial antigen, an autoantigen, and an allergen.

In a preferred embodiment of any aspects of the present invention, said nucleic acid is a RNA. In another preferred embodiment of any aspects of the present invention, said nucleic acid is a single stranded RNA (ssRNA). In a further preferred embodiment of any aspects of the present invention, said ssRNA encodes a peptide or protein of interest. In a further preferred embodiment of any aspects of the present invention, said ssRNA encodes a peptide or protein of interest, wherein said peptide or protein of interest is selected from reporter proteins and pharmaceutically active peptides or proteins. In a further preferred embodiment of any aspects of the present invention, said ssRNA encodes a peptide or protein of interest, wherein said peptide or protein of interest is a reporter protein. In a further preferred embodiment of any aspects of the present invention, said ssRNA encodes a peptide or protein of interest, wherein said peptide or protein of interest is a pharmaceutically active peptide or protein. In a further preferred embodiment of any aspects of the present invention, said ssRNA is a pharmaceutically active nucleic acid. In a further preferred embodiment of any aspects of the present invention, said ssRNA is a pharmaceutically active nucleic acid, wherein said pharmaceutically active nucleic acid is a nucleic acid that encodes a pharmaceutically active peptide or protein.

In a further preferred embodiment of any aspects of the present invention, said ssRNA is a messenger RNA (mRNA). In a further preferred embodiment of any aspects of the present invention, said mRNA encodes a peptide or protein of interest. In a further preferred embodiment of any aspects of the present invention, said mRNA encodes a peptide or protein of interest, wherein said peptide or protein of interest is selected from reporter proteins and pharmaceutically active peptides or proteins. In a further preferred embodiment of any aspects of the present invention, said mRNA encodes a peptide or protein of interest, wherein said peptide or protein of interest is a reporter protein. In a further preferred embodiment of any aspects of the present invention, said mRNA encodes a peptide or protein of interest, wherein said peptide or protein of interest is a pharmaceutically active peptide or protein. In a further preferred embodiment of any aspects of the present invention, said mRNA is a pharmaceutically active nucleic acid. In a further preferred embodiment of any aspects of the present invention, said mRNA is a pharmaceutically active nucleic acid, wherein said pharmaceutically active nucleic acid is a nucleic acid that encodes a pharmaceutically active peptide or protein.

In another preferred embodiment of any aspects of the present invention, said nucleic acid is a DNA. In a further preferred embodiment of any aspects of the present invention, said DNA is a plasmid DNA. In a further preferred embodiment of any aspects of the present invention, said pDNA encodes a peptide or protein of interest. In a further preferred embodiment of any aspects of the present invention, said pDNA encodes a peptide or protein of interest, wherein said peptide or protein of interest is selected from reporter proteins and pharmaceutically active peptides or proteins. In a further preferred embodiment of any aspects of the present invention, said pDNA encodes a peptide or protein of interest, wherein said peptide or protein of interest is a reporter protein. In a further preferred embodiment of any aspects of the present invention, said pDNA encodes a peptide or protein of interest, wherein said peptide or protein of interest is a pharmaceutically active peptide or protein. In a further preferred embodiment of any aspects of the present invention, said pDNA is a pharmaceutically active nucleic acid. In a further preferred embodiment of any aspects of the present invention, said pDNA is a pharmaceutically active nucleic acid, wherein said pharmaceutically active nucleic acid is a nucleic acid that encodes a pharmaceutically active peptide or protein.

In a further preferred embodiment of any aspects of the present invention, said nucleic acid is a pharmaceutically active nucleic acid, wherein said pharmaceutically active nucleic acid is pharmaceutically active in its own. In a further preferred embodiment of any aspects of the present invention, said nucleic acid is a pharmaceutically active nucleic acid, wherein said pharmaceutically active nucleic acid is a nucleic acid that encodes a pharmaceutically active peptide or protein.

In a preferred embodiment, said nucleic acid encodes a peptide or protein of interest, wherein said peptide or protein of interest is a reporter protein. In these embodiments, the nucleic acid comprises a reporter gene. Certain genes may be chosen as reporters because the characteristics they confer on cells or organisms expressing them may be readily identified and measured, or because they are selectable markers. Reporter genes are often used as an indication of whether a certain gene has been taken up by or expressed in the cell or organism population. Preferably, the expression product of the reporter gene is visually detectable. Common visually detectable reporter proteins typically possess fluorescent or luminescent proteins. Examples of specific reporter genes include the gene that encodes jellyfish green fluorescent protein (GFP), which causes cells that express it to glow green under blue light, the enzyme luciferase, which catalyzes a reaction with luciferin to produce light, and the red fluorescent protein (RFP). Variants of any of these specific reporter genes are possible, as long as the variants possess visually detectable properties. For example, eGFP is a point mutant variant of GFP.

In some embodiments, at least 60%, at least 70%, at least 80%, at least 90%, at least 95% or at least 99% of the LPEI in the composition is connected to the PEG fragment by a single covalent linking moiety, preferably wherein the covalent linking moiety produces a linear end-to-end linkage between the LPEI fragment and the PEG fragment. In some embodiments, at least 60% at least 70%, or at least 80%, at least 90%, at least 95% or at least 99% of the LPEI fragments comprised in the composition are comprised by said conjugate, as preferably determined by UV spectroscopy or mass spectrometry. In some embodiments, at least 60% at least 70%, or at least 80%, at least 90%, at least 95% or at least 99% of the LPEI comprised in the composition are comprised by said conjugate, as preferably determined by UV spectroscopy or mass spectrometry. In some embodiments, said composition consists essentially of said conjugate. In some embodiments, said composition consists of said conjugate.

In some embodiments, at least 60% of the LPEI in the composition is connected to a single PEG fragment by a single covalent linking moiety Z, preferably wherein the covalent linking moiety Z produces a linear end-to-end linkage between the LPEI fragment and the PEG fragment. In some embodiments, at least 60% of the LPEI fragments comprised in the composition are linked to the PEG fragment by a single triazole linker, as preferably determined by UV spectroscopy or mass spectrometry. In some embodiments, at least 70% of the LPEI in the composition is connected to the PEG fragment by a single covalent linking moiety Z, preferably wherein the covalent linking moiety Z produces a linear end-to-end linkage between the LPEI fragment and the PEG fragment. In some embodiments, at least 70% of the LPEI fragments comprised in the composition are comprised by said conjugate, as preferably determined by UV spectroscopy or mass spectrometry. In some embodiments, at least 80% of the LPEI in the composition is connected to the PEG fragment by a single covalent linking moiety Z, preferably wherein the covalent linking moiety Z produces a linear end-to-end linkage between the LPEI fragment and the PEG fragment. In some embodiments, at least 80% of the LPEI fragments comprised in the composition are comprised by said conjugate, as preferably determined by UV spectroscopy or mass spectrometry. In some embodiments, at least 90% of the LPEI in the composition is connected to the PEG fragment by a single covalent linking moiety Z, preferably wherein the covalent linking moiety Z produces a linear end-to-end linkage between the LPEI fragment and the PEG fragment. In some embodiments, at least 90% of the LPEI fragments comprised in the composition are comprised by said conjugate, as preferably determined by UV spectroscopy or mass spectrometry. In some embodiments, at least 95% of the LPEI in the composition is connected to the PEG fragment by a single covalent linking moiety Z, preferably wherein the covalent linking moiety Z produces a linear end-to-end linkage between the LPEI fragment and the PEG fragment. In some embodiments, at least 95% of the LPEI fragments comprised in the composition are comprised by said conjugate, as preferably determined by UV spectroscopy or mass spectrometry. In some embodiments, at least 99% of the LPEI in the composition is connected to the PEG fragment by a single covalent linking moiety Z, preferably wherein the covalent linking moiety Z produces a linear end-to-end linkage between the LPEI fragment and the PEG fragment. In some embodiments, at least 99% of the LPEI fragments comprised in the composition are comprised by said conjugate, as preferably determined by UV spectroscopy or mass spectrometry. In some embodiments, said composition consists essentially of said conjugate. In some embodiments, said composition consists of said conjugate. In some embodiments, the LPEI fragment does not comprise substitution beyond its first terminal end and second terminal end.

In some embodiments, the covalent linking moiety Z comprises a triazole.

In some embodiments, the Formula I* does not comprise the structure: R¹—(NH—CH₂—CH₂)ₙ—NHC(O)—(CH₂—CH₂—O)ₘ—X²-L. In some embodiments, the Formula I* does not comprise the structure R¹—(NR²—CH₂—CH₂)ₙ—NHC(O)—X¹—(O—CH₂—CH₂)ₘ—X²-L. In some embodiments, the composition does not comprise a conjugate of the structure R¹—(NH—CH₂—CH₂)ₙ—NHC(O)—X¹—(O—CH₂—CH₂)ₘ-X²-L. In some embodiments, the composition does not comprise a conjugate of the structure R¹—(NR²—CH₂—CH₂)ₙ—NHC(O)—(CH₂—CH₂—O)ₘ—X²-L.

In some embodiments, R¹ is —H.

In some embodiments, at least 80% of the R² in the composition is —H. In some embodiments, at least 85%, preferably 90%, preferably 95%, more preferably 99% of the R² in the composition is —H. In a preferred embodiment, R² is independently —H or an organic residue, wherein at least 85%, preferably 90% of said R² in said —(NR²—CH₂—CH₂)ₙ-moieties is H. In another preferred embodiment, R² is independently —H or an organic residue, wherein at least 90% of said R² in said —(NR²—CH₂—CH₂)ₙ-moieties is H. In another preferred embodiment, R² is independently —H or an organic residue, wherein at least 90% of said R² in said —(NR²—CH₂—CH₂)ₙ-moieties is H. In another preferred embodiment, R² is independently —H or an organic residue, wherein at least 91%, preferably at least 92%, more preferably 93%, of said R² in said —(NR²—CH₂—CH₂)ₙ-moieties is H. In another preferred embodiment, R² is independently —H or an organic residue, wherein at least 94%, preferably at least 95%, more preferably 96%, of said R² in said —(NR²—CH₂—CH₂)ₙ-moieties is H. In another preferred embodiment, R² is independently —H or an organic residue, wherein at least 95%, preferably wherein at least 97%, further preferably at least 98%, more preferably 99%, of said R² in said —(NR²—CH₂—CH₂)ₙ-moieties is H.

In some embodiments, Ring A is an 8-membered cycloalkenyl, 5-membered heterocycloalkyl, or 7- to 8-membered heterocycloalkenyl, wherein each cycloalkenyl, heterocycloalkyl or heterocycloalkenyl is optionally substituted at any position with one or more R^{A1}.

In some embodiments, Ring A is cyclooctene, maleimide, or 7- to 8-membered heterocycloalkenyl, wherein the heterocycloalkyl or heterocycloalkenyl does not comprise heteroatoms other than N, O and S, and wherein each cyclooctene, heterocycloalkyl or heterocycloalkenyl is optionally substituted at any position with one or more R^{A1}.

In some embodiments, Ring A is cyclooctene, maleimide, or 7- to 8-membered heterocycloalkenyl, wherein the heterocycloalkyl or heterocycloalkenyl comprises one or more heteroatoms, preferably one or two heteroatoms selected from N, O and S, and wherein each cyclooctene, heterocycloalkyl or heterocycloalkenyl is optionally substituted at any position with one or more R^{A1}.

In some embodiments, Ring A is cyclooctene, maleimide, or an 8- membered heterocycloalkene, wherein the heterocycloalkene comprises exactly one heteroatom selected from N, O, and S, wherein each cyclooctene or heterocycloalkene is optionally substituted with one or more R^{A1}.

In some embodiments, R^{A1} is —H, oxo or fluorine, or two R^{A1} combine to form one or more fused phenyl rings, preferably one or two fused phenyl rings, and wherein each phenyl ring is optionally substituted with one or more —OSO₃H or —SO₃H.

In some embodiments, Ring A is cyclooctene, maleimide, or an 8- membered heterocycloalkene, wherein the heterocycloalkene comprises exactly one heteroatom selected from N, O, and S, wherein each cyclooctene or heterocycloalkene is optionally substituted with one or more R^{A1}, wherein R^{A1} is oxo or fluorine, or wherein two R^{A1} combine to form one or more fused phenyl rings, preferably one or two fused phenyl rings.

In some embodiments, Ring A is cyclooctene, maleimide, or an 8-membered heterocycloalkene, wherein the heterocycloalkene comprises exactly one heteroatom selected from N, wherein each cyclooctene or heterocycloalkene is optionally substituted with one or two R^{A1}.

In some embodiments, R^{A1} is —H, oxo or fluorine, or two R^{A1} combine to form one or more fused phenyl rings, preferably one or two fused phenyl rings, and wherein each phenyl ring is optionally substituted with one or more R^{A2}.

In some embodiments, Ring A is cyclooctene, maleimide, or an 8-membered heterocycloalkene, wherein the heterocycloalkene comprises exactly one heteroatom selected from N, wherein each cyclooctene or heterocycloalkene is optionally substituted with one or two R^{A1}, wherein R^{A1} is —H, oxo or fluorine, or wherein two R^{A1} combine to form one or more fused phenyl rings, preferably one or two fused phenyl rings, and wherein each phenyl ring is optionally substituted with one or more —OSO₃H or —SO₃H.

In some preferred embodiments, Ring A is cyclooctene, maleimide, or an 8-membered heterocycloalkene, wherein the heterocycloalkene comprises exactly one heteroatom selected from N, wherein each cyclooctene or heterocycloalkene is optionally substituted with one or two R^{A1}, wherein R^{A1} is —H, or wherein two R^{A1} combine to form one or more fused phenyl rings, preferably one or two fused phenyl rings, and wherein each phenyl ring is optionally substituted with one or more —OSO₃H or —SO₃H.

### Preparation of Linear Conjugates

The conjugates of the invention can be prepared in a number of ways well known to those skilled in the art of polymer synthesis. By way of example, compounds of the present invention can be synthesized using the methods described below, together with synthetic methods known in the art of polymer chemistry, or variations thereon as appreciated by those skilled in the art. The methods include, but are not limited to, those methods described below. The conjugates of the present invention can be synthesized by following the steps outlined in General Schemes 1, 2, 3, 4, 5, 6, 7 and 8, or can be prepared using alternate sequences of assembling intermediates without deviating from the present invention. The conjugates of the present invention can also be synthesized using slight variations on the steps outlined below. For example, where Scheme 3 shows the use of a tetrafluorophenyl ester as an electrophilic functional group for coupling with hEGF, one of skill in the art will recognize other suitable electrophilic functional groups that can be used for the same purpose.

In some preferred embodiments, the LPEI fragment and the PEG fragment are coupled via a [3+2]cycloaddition between an azide and an alkene or alkyne to form a 1,2,3 triazole or a 4,5-dihydro-1H-[1,2,3]triazole. In some preferred embodiments, the LPEI fragment comprises the azide functional group and the PEG fragment comprises the alkene or alkyne functional group.

### LPEI Fragment

The conjugates of the present invention can comprise LPEI fragments and PEG fragments. Linear polyethyleneimine (LPEI) has the chemical formula —[NH—CH₂—CH₂]—. Thus, linear polyethyleneimine (LPEI) has the chemical formula of repeating units n of —[NH—CH₂—CH₂]—. LPEI can be synthesized according to a number of methods known in the art, including in particular the polymerization of a 2-oxazoline, followed by hydrolysis of the pendant amide bonds (see e.g., Brissault et al., Bioconjugate Chem., 2003, 14, 581-587). As noted above, the polymerization of poly(2-oxazolines) (i.e., a suitable precursor for LPEI) from 2-oxazolines can be initiated with any suitable initiator. In some embodiments, the initiator leaves an initiation residue at the alpha terminus of the poly(2-oxazoline). In a preferred embodiment, the initiation residue (i.e., R¹ of Formula I* or Formula I) is a hydrogen atom or a C₁-C₆ alkyl, preferably a hydrogen or C₁-C₄ alkyl, more preferably a hydrogen or methyl group; most preferably a hydrogen atom.). In a preferred embodiment, the initiation residue R¹ of Formula I is a hydrogen atom or a C₁-C₆ alkyl, preferably a hydrogen or C₁-C₄ alkyl, more preferably a hydrogen or methyl group; most preferably a hydrogen atom. In preferred embodiments, the initiation residue (i.e., R¹ of Formula I* or Formula I) is —H or —CH₃, most preferably —H. In a preferred embodiment, said initiation residue R¹ of Formula I* is —H. In a preferred embodiment, said initiation residue R¹ of Formula I is —H. In a preferred embodiment, said initiation residue R¹ of Formula I* is —CH₃. In a preferred embodiment, said initiation residue R¹ of Formula I is —CH₃. However, one of skill in the art will understand that the initiation residue can be the residue left from any suitable initiator capable of initiating the polymerization of poly(2-oxazolines) from 2-oxazolines.

In some embodiments, the LPEI fragment can be coupled to the PEG fragment via a [3+2]cycloaddition between an azide and an alkene or alkyne to form a 1, 2, 3 triazole or a 4,5-dihydro-1H-[1,2,3]triazole wherein the LPEI fragment comprises the azide (—N₃) functional group at the omega terminus of the chain. In some preferred embodiments, the LPEI fragment is not further substituted except for a single substitution at the alpha terminus. For example, in some preferred embodiments, the LPEI fragment comprises the repeating formula —[NH—CH₂—CH₂]—and is substituted at the omega terminus with an azide group which can be coupled to an alkyne or alkene substituent on a PEG fragment. In some preferred embodiments, the alpha terminus of the LPEI fragment can be substituted with a hydrogen atom or a C₁-C₆ alkyl, preferably a hydrogen or C₁-C₄ alkyl, more preferably a hydrogen or methyl group; most preferably a hydrogen atom.

For example, in some preferred embodiments, the LPEI fragment can be substituted at the alpha terminus with a hydrogen atom or a C₁-C₆ alkyl, preferably a hydrogen atom or C₁-C₄ alkyl, more preferably a hydrogen atom or methyl group and at the omega terminus with an azide group; in some preferred embodiments, there is no additional substitution present on the LPEI fragment. For example, conjugates of the present invention can be prepared from LPEI fragments of the following formula:

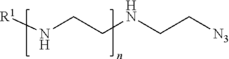

wherein R¹ can be any suitable initiation residue, preferably a hydrogen or C₁-C₆ alkyl, preferably hydrogen or C₁-C₄ alkyl, more preferably hydrogen or methyl, most preferably a hydrogen.

In some embodiments, the LPEI fragment can be terminated with a thiol group, thus, in some embodiments, the omega terminus of said LPEI fragment comprises, preferably is, a thiol group, which can be coupled to a reactive alkene group on the PEG fragment by a thiol-ene reaction. Accordingly, in some embodiments conjugates of the present invention can be prepared from LPEI fragments of the following formula:

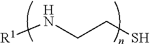

wherein R¹ can be any suitable initiation residue, preferably hydrogen or methyl, preferably a hydrogen.

In some embodiments, the LPEI fragment can be terminated with an alkene group, thus, in some embodiments, the omega terminus of said LPEI fragment comprises, preferably is, a alkene group, which can be coupled to a reactive thiol group on the PEG fragment by a thiol-ene reaction. Accordingly, in some embodiments, conjugates of the present invention can be prepared from LPEI fragments of the following formula:

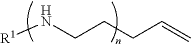

wherein R¹ can be any suitable initiation residue, preferably hydrogen or methyl, preferably a hydrogen.

The LPEI fragment can comprise a range of lengths (i.e., repeating units represented above by the variable “n”). For example, the LPEI fragment can comprise between 1 and 1000 repeating units (i.e., —NH—CH₂—CH₂—). In some embodiments, the LPEI fragment can be present as a disperse polymeric moiety and does not comprise a discrete number of —NH—CH₂—CH₂-repeating units. For example, the LPEI fragment can be present as a disperse polymeric moiety with a molecular weight of between about 5 and 50 KDa, preferably with a dispersity of about or less, preferably of about 4 or less, preferably of about 3 or less, preferably of about 2 or less, preferably of about 1.5 or less. In some embodiments, the LPEI fragment can be present as a disperse polymeric moiety with a molecular weight of between about 10 and 40 KDa with a dispersity of about 4 or less, preferably of about 3 or less, preferably of about 2 or less, preferably of about 1.5 or less. In some embodiments, the LPEI fragment can be present as a disperse polymeric moiety with a molecular weight of between about 12 and 30 KDa with a dispersity of about 3 or less, preferably of about 2 or less, preferably of about 1.5 or less. In some embodiments, the LPEI fragment can be present as a disperse polymeric moiety with a molecular weight of between about 15 and 27 KDa with a dispersity of about 2 or less, preferably of about 1.5 or less. In some embodiments, the LPEI fragment can be present as a disperse polymeric moiety with a molecular weight of between about 17 and 25 KDa, with a dispersity of about 1.2 or less.

For example, the LPEI fragment can be present as a disperse polymeric moiety comprising between about 115 and 1150 repeating units, preferably with a dispersity of about 5 or less, preferably of about 4 or less, preferably of about 3 or less, preferably of about 2 or less, preferably of about 1.5 or less. In some embodiments, the LPEI fragment can be present as a disperse polymeric moiety comprising between about 230 and 930 repeating units with a dispersity of about 4 or less, preferably of about 3 or less, preferably of about 2 or less, preferably of about 1.5 or less. In some embodiments, the LPEI fragment can be present as a disperse polymeric moiety comprising between about 280 and 700 repeating units with a dispersity of about 3 or less, preferably of about 2 or less, preferably of about 1.5 or less. In some embodiments, the LPEI fragment can be present as a disperse polymeric moiety comprising between about 350 and 630 repeating units with a dispersity of about 2 or less, preferably of about 1.5 or less. In some embodiments, the LPEI fragment can be present as a disperse polymeric moiety comprising between about 400 and 580 repeating units, with a dispersity of about 1.2 or less.

In some embodiments, said R¹—(NR²—CH₂—CH₂)ₙ-moiety is a disperse polymeric moiety with between 115 and 1150 repeating units n and a dispersity of about 5 or less, wherein preferably said R¹—(NR²—CH₂—CH₂)ₙ-moiety is a disperse polymeric moiety with between 280 and 700 repeating units n and a dispersity of about 3 or less, and wherein further preferably said R¹—(NR²—CH₂—CH₂)ₙ-moiety is a disperse polymeric moiety with between 350 and 630 repeating units n and a dispersity of about 2 or less, and again further preferably wherein said R¹—(NR²—CH₂—CH₂)ₙ-moiety is a disperse polymeric moiety with between 400 and 580 repeating units n and a dispersity of about 1.2 or less.

In a preferred embodiment, said polyethyleneimine fragment is a disperse polymeric moiety with between about 115 and about 1150 repeating units and a dispersity of about 5 or less, preferably between about 230 and about 930 repeating units with a dispersity of about 4 or less; more preferably between about 280 and about 700 repeating units with a dispersity of about 3 or less; again more preferably between about 350 and about 630 repeating units with a dispersity of about 2 or less; yet more preferably between about 400 and about 580 repeating units, with a dispersity about 1.2 or less.

In a preferred embodiment, said polyethyleneimine fragment is a disperse polymeric moiety with between about 115 and about 1150 repeating units and a dispersity of about 5 or less, preferably of about 4 or less, preferably of about 3 or less, preferably of about 2 or less, preferably of about 1.5 or less. In a preferred embodiment, said polyethyleneimine fragment is a disperse polymeric moiety with between about 230 and about 930 repeating units with a dispersity of about 4 or less, preferably of about 3 or less, preferably of about 2 or less, preferably of about 1.5 or less. In a preferred embodiment, said polyethyleneimine fragment is a disperse polymeric moiety with between about 280 and about 700 repeating units with a dispersity of about 3 or less, preferably of about 2 or less, preferably of about 1.5 or less. In a preferred embodiment, said polyethyleneimine fragment is a disperse polymeric moiety with between about 350 and about 630 repeating units with a dispersity of about 2 or less, preferably of about 1.5 or less. In a preferred embodiment, said polyethyleneimine fragment is a disperse polymeric moiety with between about 400 and about 580 repeating units, with a dispersity about 1.2 or less.

As noted above, one of skill in the art will understand that in some embodiments, the LPEI fragment may include organic residues, (i.e., pendant amide groups) connected at the nitrogen atoms embedded within the LPEI chain. One of skill in the art will understand that such organic residues (i.e., amide groups) can be formed during the ring-opening polymerization of 2-oxazolines to form a poly(2-oxazoline). Without wishing to be bound by theory, LPEI can be formed from a poly(2-oxazoline) by cleavage of the amide groups (e.g., using an acid such as HCl). However, in some cases not every amide linkage may be cleaved under these conditions. Accordingly, in some embodiments about 5% or less of the nitrogen atoms in the LPEI fragment may be connected to an organic residue to form an amide. In some embodiments, about 4% or less, about 3% or less, about 2% or less, about 1% or less, about 0.5% or less, about 0.4% or less, about 0.3% or less, about 0.2% or less, or about 0.1% or less of the nitrogen atoms in the LPEI fragment may be connected to an organic residue to form an amide. One of skill in the art will understand that the molecular weight of the LPEI fragment includes the percentage of LPEI fragment that is bonded to an organic residue as an amide. Moreover, one of skill in the art will understand that although chemical structures drawn herein show repeating —NH—CH₂—CH₂—fragments, trace amounts of residual organic residue such as pendant amide groups (e.g., those defined above) may still be present in the resulting triconjugates or polyplexes of the present disclosure. The term “triconguate”, as occasionally used herein, shall refer to the inventive conjugate. The prefix “tri-” is caused by the three components comprised by the inventive conjugates, namely the LPEI fragment, the PEG fragment and the targeting fragment.

### PEG Fragment

Polyethylene glycol (PEG) has the chemical formula —[O—CH₂—CH₂]—. Thus, polyethylene glycol (PEG) has the chemical formula of repeating units m of —[O—CH₂—CH₂]—. In some preferred embodiments, the PEG fragment can be coupled to the LPEI fragment via a [3+2]cycloaddition between an azide and an alkene or alkyne to form a 1,2,3 triazole or a 4,5-dihydro-1H-[1,2,3]triazole, wherein the respective reactive precursor molecule comprising the PEG fragment further comprises the alkene or alkyne functional group. For example, in some preferred embodiments, the reactive precursor molecule comprising the PEG fragment comprises the repeating formula —[O—CH₂—CH₂]—and is substituted at a first end (i.e., terminus) with an alkene or alkyne group (e.g., via a linking moiety “X¹” as discussed herein) which can be coupled to the azide group of a corresponding respective reactive precursor molecule comprising the LPEI fragment.

In some preferred embodiments, said alkene or alkyne group is an activated alkene or alkyne group capable of spontaneously reacting with an azide (e.g., without the addition of a catalyst such as a copper catalyst). For example, an activated alkyne group can be incorporated into a 7- or 8-membered ring, resulting in a strained species that reacts spontaneously with the azide group of the LPEI fragment. An activated alkene can include a maleimide moiety, wherein the alkene is activated by conjugation to the neighboring carbonyl groups. In some preferred embodiments, the second end (i.e., terminus) of the PEG fragment can be substituted with a targeting fragment (e.g., hEGF, HER2, folate, or DUPA) (e.g., via a linking moiety “X²” as discussed herein).

The PEG fragment can comprise a range of lengths (i.e., repeating units represented by the variable “m”). In other embodiments, the PEG fragment can comprise a discrete number of repeating —O—CH₂—CH₂—units and is not defined in terms of an average chain length. In a preferred embodiment, said —(O—CH₂—CH₂)ₘ- is a disperse polymeric moiety. In a preferred embodiment, said —(O—CH₂—CH₂)ₘ-moiety comprises, preferably consists of, a discrete number of repeating units m. In a preferred embodiment, said —(O—CH₂—CH₂)ₘ-moiety comprises, preferably consists of, a discrete number of contiguous repeating units m.

In some preferred embodiments, the PEG fragment is a disperse polymeric moiety comprising between about 1 and about 200 repeating units, preferably between about 1 and about 200 repeating units. In some preferred embodiments, the PEG fragment can comprise between 1 and 100 repeating units (i.e., —O—CH₂—CH₂—). Preferably the PEG fragments of the present invention comprise between about 1 and about 100 repeating units, between about 1 and about 90 repeating units, between about 1 and about 80 repeating units, between about 1 and about 70 repeating units, between about 1 and about 60 repeating units, between about 1 and about 50 repeating units, between about 1 and about 50 repeating units, between about 1 and about 40 repeating units, between about 1 and about 30 repeating units, or between about 1 and about 20 repeating units. In some other preferred embodiments, the PEG fragments comprise a discrete number of repeating units m, preferably 12 repeating units or 24 repeating units. In some embodiment, said polyethylene glycol fragment is a disperse polymeric moiety with between about 2 and about 80 repeating units and a dispersity of about 2.0 or less, preferably of about 1.8 or less, further of about 1.5 or less; preferably between about 2 and about 70 repeating units with a dispersity of about 1.8 or less, preferably of about 1.5 or less; more preferably between about 2 and about 50 repeating units with a dispersity of about 1.5 or less. In some embodiment, said —(O—CH₂—CH₂)ₘ-moiety is a disperse polymeric moiety with between about 2 and about 80 repeating units and a dispersity of about 2.0 or less, preferably between about 2 and about 70 repeating units with a dispersity of about 1.8 or less; more preferably between about 2 and about 50 repeating units with a dispersity of about 1.5 or less.

In a preferred embodiment, said polyethylene glycol fragment PEG fragment comprises, preferably consists of, a discrete number of repeating units m, preferably of 12 or 24 repeating units. In a preferred embodiment, said m (of said —(O—CH₂—CH₂)ₘ-moiety) comprises, preferably consists of, a discrete number of repeating units m, preferably of 12 or 24 repeating units.

In a preferred embodiment, the PEG fragment comprise, preferably consist of, a discrete number of repeating units m of 2 to 100, preferably of a discrete number of repeating units m of 4 to 60. In a preferred embodiment, the PEG fragment comprise, preferably consist of, a discrete number of repeating units m of 4 to 60, preferably of a discrete number of repeating units m of 10 to 60. In a preferred embodiment, the PEG fragment comprise, preferably consist of, a discrete number of repeating units m of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In a preferred embodiment, the PEG fragment comprise, preferably consist of, a discrete number of repeating units m of 4, 8, 12, 16, 20, 24, 28, 32, 36, 40, 44, 48, 52, 56, or 60. In a preferred embodiment, the PEG fragment comprise, preferably consist of, a discrete number of repeating units m of 4. In a preferred embodiment, the PEG fragment comprise, preferably consist of, a discrete number of repeating units m of 12. In a preferred embodiment, the PEG fragment comprise, preferably consist of, a discrete number of repeating units m of 24. In a preferred embodiment, the PEG fragment comprise, preferably consist of, a discrete number of repeating units m of 36.

In a preferred embodiment, the PEG fragment comprise, preferably consist of, a discrete number of contiguous repeating units m of 2 to 100, preferably of a discrete number of contiguous repeating units m of 4 to 60. In a preferred embodiment, the PEG fragment comprise, preferably consist of, a discrete number of contiguous repeating units m of 4 to 60, preferably of a discrete number of contiguous repeating units m of 10 to 60. In a preferred embodiment, the PEG fragment comprise, preferably consist of, a discrete number of contiguous repeating units m of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In a preferred embodiment, the PEG fragment comprise, preferably consist of, a discrete number of contiguous repeating units m of 4, 8, 12, 16, 20, 24, 28, 32, 36, 40, 44, 48, 52, 56, or 60. In a preferred embodiment, the PEG fragment comprise, preferably consist of, a discrete number of contiguous repeating units m of 4. In a preferred embodiment, the PEG fragment comprise, preferably consist of, a discrete number of contiguous repeating units m of 12. In a preferred embodiment, the PEG fragment comprise, preferably consist of, a discrete number of contiguous repeating units m of 24. In a preferred embodiment, the PEG fragment comprise, preferably consist of, a discrete number of contiguous repeating units m of 36.

In a preferred embodiment, said —(O—CH₂—CH₂)ₘ-moiety of Formula I* or Formula I comprise, preferably consist of, a discrete number of repeating units m of 2 to 100, preferably of a discrete number of repeating units m of 4 to 60. In a preferred embodiment, said —(O—CH₂—CH₂)ₘ-moiety comprise, preferably consist of, a discrete number of repeating units m of 4 to 60, preferably of a discrete number of repeating units m of 10 to 60. In a preferred embodiment, said —(O—CH₂—CH₂)ₘ-moiety comprise, preferably consist of, a discrete number of repeating units m of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In a preferred embodiment, said —(O—CH₂—CH₂)ₘ-moiety comprise, preferably consist of, a discrete number of repeating units m of 4, 8, 12, 16, 20, 24, 28, 32, 36, 40, 44, 48, 52, 56, or 60. In a preferred embodiment, said —(O—CH₂—CH₂)ₘ-moiety comprise, preferably consist of, a discrete number of repeating units m of 4. In a preferred embodiment, said —(O—CH₂—CH₂)ₘ-moiety comprise, preferably consist of, a discrete number of repeating units m of 12. In a preferred embodiment, said —(O—CH₂—CH₂)ₘ-moiety comprise, preferably consist of, a discrete number of repeating units m of 24. In a preferred embodiment, said —(O—CH₂—CH₂)ₘ-moiety comprise, preferably consist of, a discrete number of repeating units m of 36.

In a preferred embodiment, said —(O—CH₂—CH₂)ₘ-moiety of Formula I* or Formula I comprise, preferably consist of, a discrete number of contiguous repeating units m of 2 to 100, preferably of a discrete number of contiguous repeating units m of 4 to 60. In a preferred embodiment, said —(O—CH₂—CH₂)ₘ-moiety comprise, preferably consist of, a discrete number of contiguous repeating units m of 4 to 60, preferably of a discrete number of contiguous repeating units m of 10 to 60. In a preferred embodiment, said —(O—CH₂—CH₂)ₘ-moiety comprise, preferably consist of, a discrete number of contiguous repeating units m of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In a preferred embodiment, said —(O—CH₂—CH₂)ₘ-moiety comprise, preferably consist of, a discrete number of contiguous repeating units m of 4, 8, 12, 16, 20, 24, 28, 32, 36, 40, 44, 48, 52, 56, or 60. In a preferred embodiment said —(O—CH₂—CH₂)ₘ-moiety comprise, preferably consist of, a discrete number of contiguous repeating units m of 4. In a preferred embodiment, said —(O—CH₂—CH₂)ₘ-moiety comprise, preferably consist of, a discrete number of contiguous repeating units m of 12. In a preferred embodiment, said —(O—CH₂—CH₂)ₘ-moiety comprise, preferably consist of, a discrete number of contiguous repeating units m of 24. In a preferred embodiment, said —(O—CH₂—CH₂)ₘ-moiety comprise, preferably consist of, a discrete number of contiguous repeating units m of 36.

In preferred embodiments, the PEG fragment comprised in the inventive conjugates and compositions comprises, preferably consists of, a discrete number m of repeating —(O—CH₂—CH₂)-units and is not defined in terms of an average chain length. Thus, the PEG fragment comprised in the inventive conjugates and compositions comprises, preferably consists of, a discrete number m of repeating —(O—CH₂—CH₂)-units and is not defined in terms of an average chain length but has a specifically defined discrete molecular weight associated with the discrete number m of repeating —(O—CH₂—CH₂)-units. In a preferred embodiment, said PEG fragment comprises, preferably consists of, a discrete number m of repeating units —(O—CH₂—CH₂)-units, wherein typically and preferably said discrete number (m) is a discrete number (m) of and between 25 to 100, further preferably of and between 25 to 60. In a preferred embodiment, said PEG fragment comprises, preferably consists of, a discrete number m of contiguous repeating units —(O—CH₂—CH₂)-units, wherein typically and preferably said discrete number (m) is a discrete number (m) of and between 25 to 100, further preferably of and between 25 to 60.

The expressions “polyethylene glycol fragment comprising a discrete number (m) of repeating —(O—CH₂—CH₂)—units”, or “PEG fragment comprising a discrete number (m) of repeating —(O—CH₂—CH₂)—units” shall refer to a fragment comprising, preferably consisting of, a discrete number—typically herein referred to a discrete number m—of repeating —(O—CH₂—CH₂)—units, wherein said discrete number (m) is a discrete, i.e. specific and single defined and integer, number (m) of 25 to 100, preferably of 25 to 60. Thus, the expressions “polyethylene glycol fragment comprising a discrete number (m) of repeating —(O—CH₂—CH₂)—units”, or “PEG fragment comprising a discrete number (m) of repeating —(O—CH₂—CH₂)—units” shall refer to a fragment comprising, preferably consisting of, a discrete number m—of repeating —(O—CH₂—CH₂)—units, wherein said discrete number (m) is a discrete, i.e. specific and single defined and integer, number (m) of 25 to 100, preferably of 25 to 60, and thus said defined PEG fragments comprise, preferably consist of, a discrete number m of repeating —(O—CH₂—CH₂)—units and are not defined in terms of an average chain length but they each have a specifically defined discrete molecular weight. When herein referring to a discrete number of 25 to 100, it shall refer to any integer of and between 25 to 100, i.e. any integer between 25 and 100 including the integer and discrete numbers mentioned as borders such as here 25 and 100. By way of further example, a PEG fragment comprising a discrete number (m) of repeating —(O—CH₂—CH₂)—units, wherein said discrete number m is 36, refers to a PEG fragment comprising a chain of —(O—CH₂—CH₂)-units that contains exactly 36 —(O—CH₂—CH₂)—units. Such chain of exactly 36 —(O—CH₂—CH₂)-units is abbreviated as PEG₃₆. Such PEG fragment is in contrast to a “polymeric PEG fragment”, a “polydisperse PEG fragment” or a “disperse PEG fragment”, which refers to a heteregenous mixture of sizes and molecular weights as the result of a polymer reaction, typically in a Poisson distribution (J Herzberger et al.; Chem Rev, 2016, 116:2170-2243). The PEG fragments of the present invention comprising a discrete number (m) of repeating —(O—CH₂—CH₂)—units are not synthesized via a polymerization process. The PEG fragments of the present invention comprise a discrete number (m) of repeating —(O—CH₂—CH₂)—units and are single molecule fragments with a discrete, i.e. defined and specified, chain length. Thus, the PEG fragments of the present invention comprising a discrete number (m) of repeating —(O—CH₂—CH₂)—units are single molecule fragments with a discrete, i.e. defined and specified chain length. The PEG fragments of the present invention are not a mixture of molecular entities (such as those resulting from a random polymerization reaction). The discreteness of the inventive discrete PEG fragments distinguishes them from the polydisperse art.

The PEG fragments of the present invention may comprise, preferably consist of, homogenous discrete PEG fragments or heterogeneous discrete PEG fragments, typically and preferably homogenous discrete PEG fragments. The term “homogenous discrete PEG fragments”, as used herein, means a discrete PEG structure whose entire chemical backbone is made up of a continuous and contiguous and specific discrete number of only ethylene oxide units. In other words, no other functionality is present within said homogenous discrete PEG fragments. The termini of the respective reactive precursor molecules comprising homogeneous discrete PEG fragments, however, can and typically do have, for the sake of conjugation with the PEI fragments and the targeting fragments, functional groups. The term “heterogeneous discrete PEG fragments”, as used herein, means a discrete PEG structure wherein the basic ethylene oxide backbone comprising a discrete number of ethylene oxide units is broken up by or substituted with other functional groups or units within its structure such as, for example, the inclusion of amide or ester bonds or other functional units. In preferred embodiments of the present invention, the PEG fragment is a homogenous discrete PEG fragment.

In some preferred embodiments, the PEG fragment can be coupled to the LPEI fragment via a [3+2]cycloaddition between an azide and an alkene or alkyne to form a 1,2,3 triazole or a 4,5-dihydro-1H-[1,2,3]triazole, wherein the respective reactive precursor molecule comprising the PEG fragment further comprises the alkene or alkyne functional group. For example, in some preferred embodiments, the reactive precursor molecule comprising the PEG fragment comprises the repeating formula —[O—CH₂—CH₂]—and is substituted at a first end (i.e., terminus) with an alkene or alkyne group (e.g., via a linking moiety “X¹” as discussed herein) which can be coupled to the azide group of a corresponding respective reactive precursor molecule comprising the LPEI fragment. In some preferred embodiments, said alkene or alkyne group is an activated alkene or alkyne group capable of spontaneously reacting with an azide (e.g., without the addition of a catalyst such as a copper catalyst). For example, an activated alkyne group can be incorporated into a 7- or 8-membered ring, resulting in a strained species that reacts spontaneously with the azide group of the LPEI fragment.

The PEG fragment comprised in the inventive conjugates and compositions comprises, preferably consists of, a discrete number m of repeating —O—CH₂—CH₂—units and is not defined in terms of an average chain length, as it is the case for polymeric PEG fragments. In a preferred embodiment, said —(O—CH₂—CH₂)ₘ- units comprise, preferably consist of, a discrete number of repeating units m. In a preferred embodiment, said —(O—CH₂—CH₂)ₘ- units comprise, preferably consist of, a discrete number of contiguous repeating units m.

In a preferred embodiment, the PEG fragment comprise, preferably consist of, a discrete number of repeating units m of 25 to 100, preferably of a discrete number of repeating units m of 25 to 60. In a preferred embodiment, the PEG fragment comprise, preferably consist of, a discrete number of repeating units m of 25 to 60, preferably of a discrete number of repeating units m of 30 to 50. In a preferred embodiment, the PEG fragment comprise, preferably consist of, a discrete number of repeating units m of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. The synthesis of said PEG fragments comprising or consisting of discrete numbers repeating —(O—CH₂—CH₂)ₘ—units and thus discrete PEGs are described in WO2004/073620 and WO2013/033476. In a preferred embodiment, the PEG fragment comprise, preferably consist of, a discrete number of repeating units m of 28, 32, 36, 40, 44, 48, 52, 56, or 60. In a preferred embodiment, the PEG fragment comprise, preferably consist of, a discrete number of repeating units m of 28. In a preferred embodiment, the PEG fragment comprise, preferably consist of, a discrete number of repeating units m of 32. In a preferred embodiment, the PEG fragment comprise, preferably consist of, a discrete number of repeating units m of 36. In a preferred embodiment, the PEG fragment comprise, preferably consist of, a discrete number of repeating units m of 40. In a preferred embodiment, the PEG fragment comprise, preferably consist of, a discrete number of repeating units m of 44. In a preferred embodiment, the PEG fragment comprise, preferably consist of, a discrete number of repeating units m of 48.

In a preferred embodiment, the PEG fragment comprise, preferably consist of, a discrete number of contiguous repeating units m of 25 to 100, preferably of a discrete number of contiguous repeating units m of 25 to 60. In a preferred embodiment, the PEG fragment comprise, preferably consist of, a discrete number of contiguous repeating units m of 25 to 60, preferably of a discrete number of contiguous repeating units m of 30 to 50. In a preferred embodiment, the PEG fragment comprise, preferably consist of, a discrete number of contiguous repeating units m of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In a preferred embodiment, the PEG fragment comprise, preferably consist of, a discrete number of contiguous repeating units m of 28, 32, 36, 40, 44, 48, 52, 56, or 60. In a preferred embodiment, the PEG fragment comprise, preferably consist of, a discrete number of contiguous repeating units m of 28. In a preferred embodiment, the PEG fragment comprise, preferably consist of, a discrete number of contiguous repeating units m of 32. In a preferred embodiment, the PEG fragment comprise, preferably consist of, a discrete number of contiguous repeating units m of 36. In a preferred embodiment, the PEG fragment comprise, preferably consist of, a discrete number of contiguous repeating units m of 40. In a preferred embodiment, the PEG fragment comprise, preferably consist of, a discrete number of contiguous repeating units m of 44. In a preferred embodiment, the PEG fragment comprise, preferably consist of, a discrete number of contiguous repeating units m of 48.

In a preferred embodiment, said —(O—CH₂—CH₂)ₘ-moiety of Formula I* or Formula I consists of a discrete number of repeating units m of 25 to 100, preferably of a discrete number of repeating units m of 25 to 60. In a preferred embodiment, said —(O—CH₂—CH₂)ₘ-moiety consists of a discrete number of repeating units m of 25 to 60, preferably of a discrete number of repeating units m of 30 to 50. In a preferred embodiment, said —(O—CH₂—CH₂)ₘ-moiety consists of a discrete number of repeating units m of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In a preferred embodiment, said —(O—CH₂—CH₂)ₘ-moiety consists of a discrete number of repeating units m of 28, 32, 36, 40, 44, 48, 52, 56, or 60. In a preferred embodiment, said —(O—CH₂—CH₂)ₘ-moiety consists of a discrete number of repeating units m of 28. In a preferred embodiment, said —(O—CH₂—CH₂)ₘ-moiety consists of a discrete number of repeating units m of 32. In a preferred embodiment, said —(O—CH₂—CH₂)ₘ-moiety consists of a discrete number of repeating units m of 36. In a preferred embodiment, said —(O—CH₂—CH₂)ₘ-moiety consists of a discrete number of repeating units m of 40. In a preferred embodiment, said —(O—CH₂—CH₂)ₘ-moiety consists of a discrete number of repeating units m of 44. In a preferred embodiment, said —(O—CH₂—CH₂)ₘ-moiety consists of a discrete number of repeating units m of 48.

In a preferred embodiment, said —(O—CH₂—CH₂)ₘ-moiety of Formula I* or Formula I consists of a discrete number of contiguous repeating units m of 25 to 100, preferably of a discrete number of contiguous repeating units m of 25 to 60. In a preferred embodiment, said —(O—CH₂—CH₂)ₘ-moiety consists of a discrete number of contiguous repeating units m of 25 to 60, preferably of a discrete number of contiguous repeating units m of 30 to 50. In a preferred embodiment, said —(O—CH₂—CH₂)ₘ-moiety consists of a discrete number of contiguous repeating units m of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In a preferred embodiment, said —(O—CH₂—CH₂)ₘ-moiety consists of a discrete number of contiguous repeating units m of 28, 32, 36, 40, 44, 48, 52, 56, or 60. In a preferred embodiment said —(O—CH₂—CH₂)ₘ-moiety consists of a discrete number of contiguous repeating units m of 28. In a preferred embodiment, said —(O—CH₂—CH₂)ₘ-moiety consists of a discrete number of contiguous repeating units m of 32. In a preferred embodiment, said —(O—CH₂—CH₂)ₘ-moiety consists of a discrete number of contiguous repeating units m of 36. In a preferred embodiment, said —(O—CH₂—CH₂)ₘ-moiety consists of a discrete number of contiguous repeating units m of 40. In a preferred embodiment, said —(O—CH₂—CH₂)ₘ-moiety consists of a discrete number of contiguous repeating units m of 44. In a preferred embodiment, said —(O—CH₂—CH₂)ₘ-moiety consists of a discrete number of contiguous repeating units m of 48.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

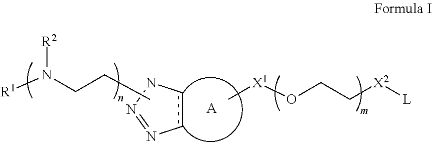

wherein:
[symbol] is a single bond or a double bond;
n is any integer between 1 and 1500, preferably any integer between 2 and 1500;
m is a discrete number of repeating units m of 2 to 100, preferably of a discrete number of repeating units m of 4 to 60, and wherein preferably said discrete number m is a discrete number of contiguous repeating —(O—CH₂—CH₂)—units, and wherein said discrete number of contiguous repeating —(O—CH₂—CH₂)—units) is any discrete number of 2 to 100, preferably of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

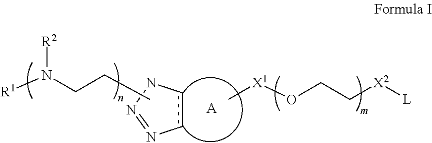

wherein:
[symbol] is a single bond or a double bond;
n is any integer between 1 and 1500, preferably any integer between 2 and 1500;
m is a discrete number of repeating units m of 2 to 100, preferably of a discrete number of repeating units m of 4 to 60, and wherein preferably said discrete number m is a discrete number of contiguous repeating —(O—CH₂—CH₂)—units, and wherein said discrete number of contiguous repeating —(O—CH₂—CH₂)—units) is any discrete number of 2 to 100, preferably of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In some preferred embodiments, the conjugates of the present invention comprise an LPEI fragment present as a disperse polymeric moiety, wherein n is between about 280 and about 700 with a dispersity of about 3 or less, preferably between about 350 and about 630 with a dispersity of about 2 or less, and more preferably between about 400 and 580 with a dispersity about 1.2 or less, and wherein said conjugates of the present invention further comprise an PEG fragment present (i) as a disperse polymeric moiety, wherein m is between about 2 and about 80 and a dispersity of about 2 or less, preferably between about 2 and about 70 with a dispersity of about 1.8 or less; more preferably between about 2 and about 50 repeating units with a dispersity of about 1.5, or (ii) as a discrete number of repeating units m, wherein preferably discrete number of repeating units m are 12 or 24 repeating units.

In some embodiments, the conjugates of the present invention comprise an LPEI fragment present as a disperse polymeric moiety of about 17 and 25 KDa, with a dispersity of about 1.2 or less and a PEG fragment comprising, preferably consisting of, 12 repeating units. In some preferred embodiments, the conjugates of the present invention can comprise an LPEI fragment present as a disperse polymeric moiety with a molecular weight of between about 17 and 25 KDa, with a dispersity of about 1.2 or less and a PEG fragment, preferably consisting of, 24 repeating units.

### Targeting Fragment

The inventive conjugates comprise a targeting fragment which allows to direct the inventive conjugate and the inventive polyplex to a particular target cell type, collection of cells, organ or tissue. Typically and preferably, the targeting fragment is capable of binding to a target cell, preferably to a cell receptor or cell surface receptor thereof.

As used herein, the term “cell surface receptor”, as used herein refers to a protein, glycoprotein or lipoprotein which is present at the surface of the cell, and which is typically and preferably a distinctive marker for the recognition of a cell. Typically and preferably, said cell surface receptor is able to bind to a ligand which include hormones, neurotransmitters, cytokines, growth factors, cell adhesion molecules, or nutrients, in the form of peptides, small molecules, saccharides and oligosaccharides, lipids, amino acids, and such other binding moieties such as antibodies, aptamers, affibodies, antibody fragments and the like.

The inventive conjugate and polyplex comprising the targeting fragment is aiming to mimic such ligand-receptor interaction. Thus, in a preferred embodiment, said targeting fragment is capable of binding to a cell surface receptor. In a preferred embodiment, said cell surface receptor is selected from a growth factor receptor, an extracellular matrix protein, a peripheral membrane protein, a transmembrane protein, preferably transmembrane protein of type II, a cytokine receptor, a hormone receptor, a glycosylphosphatidylinositol (GPI) anchored membrane protein, a carbohydrate-binding integral membrane protein, an asialoglycoprotein receptor (ASGPr), a lectin, an ion channel, a G-protein coupled receptor, and an enzyme-linked receptor such as a tyrosine kinase-coupled receptor.

In a preferred embodiment, said targeting fragment is capable of binding to a cell surface receptor. In a preferred embodiment, said cell surface receptor is selected from a growth factor receptor, an extracellular matrix protein, a peripheral membrane protein, a transmembrane protein, preferably transmembrane protein of type II, a cytokine receptor, a hormone receptor, a glycosylphosphatidylinositol (GPI) anchored membrane protein, a carbohydrate-binding integral membrane protein a lectin, an ion channel, a G-protein coupled receptor, and an enzyme-linked receptor such as a tyrosine kinase-coupled receptor. In a preferred embodiment, said cell surface receptor is a growth factor receptor. In a preferred embodiment, said cell surface receptor is an extracellular matrix protein. In a preferred embodiment, said cell surface receptor is a cytokine receptor. In a preferred embodiment, said cell surface receptor is a hormone receptor. In a preferred embodiment, said cell surface receptor is a glycosylphosphatidylinositol (GPI) anchored membrane protein. In a preferred embodiment, said cell surface receptor is a carbohydrate-binding integral membrane protein. In a preferred embodiment, said cell surface receptor is a lectin. In a preferred embodiment, said cell surface receptor is an ion channel. In a preferred embodiment, said cell surface receptor is an enzyme-linked receptor, wherein preferably said enzyme-linked receptor is a tyrosine kinase-coupled receptor. In a preferred embodiment, said cell surface receptor is a peripheral membrane protein. In a preferred embodiment, said cell surface receptor is a transmembrane protein. In a preferred embodiment, said cell surface receptor is a transmembrane protein of type II.

In a preferred embodiment, said cell surface receptor is selected from an epidermal growth factor receptor (EGFR), human epidermal growth factor receptor 2 (HER2), prostate specific membrane antigen (PSMA), an insulin-like growth factor 1 receptor (IGF1R), a vascular endothelial growth factor receptor (VEGFR), a platelet-derived growth factor receptor (PDGFR) and a fibroblast growth factor receptor (FGFR). In a preferred embodiment, said cell surface receptor is an epidermal growth factor receptor (EGFR). In a preferred embodiment, said cell surface receptor is a human epidermal growth factor receptor 2 (HER2). In a preferred embodiment, said cell surface receptor is a prostate specific membrane antigen (PSMA). In a preferred embodiment, said cell surface receptor is an insulin-like growth factor 1 receptor (IGF1R). In a preferred embodiment, said cell surface receptor is a vascular endothelial growth factor receptor (VEGFR). In a preferred embodiment, said cell surface receptor is a platelet-derived growth factor receptor (PDGFR). In a preferred embodiment, said cell surface receptor is a fibroblast growth factor receptor (FGFR).

The targeting fragment in accordance with the present invention aims to locate and to deliver, in particular to selectively deliver, the inventive polyplexes and payloads such as the nucleic acids to the desired target, in particular to the desired target cell. In addition, the inventive conjugate comprising said targeting fragment not only allows to selectively deliver the conjugate and polyplex to a target such as a target cell, but, in addition, allows to enable internalization and to facilitate selective cellular uptake of the polyanion payload and nucleic acid payload, respectively, by the target, in particular by the target cell. Thus, the targeting fragment in accordance with the present invention represents a portion of the inventive conjugate and polyplex that is capable of specific binding to a selected target, preferably to a selected target cell, further preferably to a cell receptor.

In a preferred embodiment, said targeting fragment is capable of binding to a target cell. In a preferred embodiment, said targeting fragment is capable of binding to a selected target cell type. In a preferred embodiment, said targeting fragment is capable of binding to a target cell receptor. In a preferred embodiment, said targeting fragment is capable of binding to a target cell surface receptor.

In a preferred embodiment, said targeting fragment functions to bind to a target cell. In a preferred embodiment, said targeting fragment functions to bind to a selected target cell type. In a preferred embodiment, said targeting fragment functions to bind to a target cell receptor, In a preferred embodiment, said targeting fragment functions to bind to a target cell surface receptor.

In a preferred embodiment, said targeting fragment is capable of specifically binding to a target cell. In a preferred embodiment, said targeting fragment is capable of specifically binding to a selected target cell type. In a preferred embodiment, said targeting fragment is capable of specifically binding to a target cell receptor. In a preferred embodiment, said targeting fragment is capable of specifically binding to a target cell surface receptor.

In one embodiment, said specifically binding to a target cell, to a target cell or to a target cell surface receptor, means that the targeting fragment and the inventive conjugate and/or inventive polyplex, respectively, binds to said target cell, said target cell receptor, said target cell surface receptor, at least twice, preferably at least three times, further preferably at least four times, again further preferably at least five times as strong as it binds to other non-targeted cells, cell receptors, cell surface receptors, typically and preferably measured by the dissociation constant (KD). Preferably, a targeting fragment binds to the selected cell surface receptor with a KD of less than 10⁻⁵ M, preferably less than 10⁻⁶ M, more preferably less than 10⁻⁷ M and even more preferably less than 108 M.

In one embodiment, said specifically binding to a target cell, to a target cell receptor or to a target cell surface receptor means that the targeting fragment and the inventive conjugate and/or inventive polyplex, respectively, binds to said target cell, said target cell receptor or said target cell surface receptor at least twice, preferably at least three times, further preferably at least five times, again further preferably at least ten times, further preferably at least hundred times as strong as the corresponding conjugate and/or polyplex that is identical to the inventive conjugate and/or the inventive polyplex but comprises instead of the targeting fragment a non-specific fragment such as an hydroxyl group or a —OMe moiety, preferably the —OMe moiety. The binding to the target cell, to the target cell receptor or to the target cell surface receptor is typically and preferably measured by the dissociation constant (KD). Preferably, a targeting fragment binds to the selected target cell surface receptor with a KD of less than 10⁻⁵ M, preferably less than 10⁻⁶ M, more preferably less than 10⁻⁷ M and even more preferably less than 10⁻⁸ M. In a preferred embodiment, said binding or said specific binding, and thus the level of binding of the inventive conjugate and inventive polyplex, respectively, can be determined by binding assays or displacement assays or by FRET or other measures demonstrating interaction between the targeting fragment and the cell receptor, preferably the cell surface receptor.

The term “binding”, as used herein with reference to the binding of the targeting fragment to a cell, a cell receptor or a cell surface receptor refers preferably to interactions via non-covalent binding, such as electrostatic interactions, van der Waals interaction, hydrogen bonds, hydrophobic interactions, ionic bonds, charge interactions, affinity interactions, and/or dipole-dipole interactions.

In another embodiment, said specifically binding to a target cell, to a target cell receptor or to a target cell surface receptor results in a biological effect which is caused by said specific binding of the targeting fragment and inventive conjugate and/or the inventive polyplex, respectively, and/or is caused by the delivered inventive conjugate and/or polyplex and polyanion payload and nucleic acid payload, respectively, which biological effect is at least 2-fold, preferably at least 3-fold, further preferably at least 5-fold and again further preferably at least 10-fold, and again further preferably at least 25-fold, at least 50-fold or at least 100-fold greater, as compared to said biological effect of a non-targeted cell, a non-targeted cell receptor or a non-targeted cell surface receptor.

In another embodiment, said specifically binding to a target cell, to a target cell receptor, or to a target cell surface receptor results in a biological effect which is caused by said specific binding of the targeting fragment and inventive conjugate and/or the inventive polyplex, respectively, and/or is caused by the delivered inventive conjugate and/or polyplex and polyanion payload and nucleic acid payload, respectively, which biological effect is is at least 2-fold, preferably at least 3-fold, further preferably at least 5-fold and again further preferably at least 10-fold, and again further preferably at least 25-fold, at least 50-fold or at least 100-fold greater, as compared to said biological effect caused by the corresponding conjugate and/or polyplex that is identical to the inventive conjugate and/or the inventive polyplex but comprises instead of the targeting fragment a non-specific fragment such as an hydroxyl group or a —OMe moiety, preferably the —OMe moiety.

The binding and specific binding can be determined as well by measures of activation of protein signalling and therefore can be measured by protein phosphorylation or protein expression, mRNA expression in cells or tissues (using westernblot analysis, real time PCR, RNAseq IHC etc). The level of delivery of an inventive polyplex to a particular tissue may be measured by comparing the amount of protein produced in a cell with overexpression vs a cell with normal and low expression by means of western blot analysis or luminescence/fluorescent assay, flow cytometry assays or measuring the secretion of the protein by measures of such as ELISA, ECLIA. By comparing the amount of expression or secretion of a downstream protein (from the nucleic acid delivered such as polyIC) in cells/tissues with overexpression of the target receptor as compared to normal cells/tissues or cells/tissues with low expression by means of western blot analysis or luminescence/fluorescent assay, flow cytometry assays or measuring the secretion of the protein by measures of such as ELISA, ECLIA. The level of delivery can also be measured by means of cytotoxicity using cell survival assays or cell death assays including (MTT, Methylene Blue assays, CellTiter-Glo assays, propidium iodide assay). By comparing the amount of protein produced in a tissue to the weight of said tissue, comparing the amount of therapeutic and/or prophylactic in a tissue to the weight of said tissue, comparing the amount of protein produced in a tissue to the amount of total protein in said tissue, or comparing the amount of therapeutic and/or prophylactic in a tissue to the amount of total therapeutic and/or prophylactic in said tissue. It will be understood that the delivery of an inventive polyplex to a target cell or target tissue need not be determined in a subject being treated, it may be determined in a surrogate such as an animal model or a cellular model.

Thus, in a preferred embodiment, said biological effect is selected from (i) activation of protein signalling, (ii) protein expression, (iii) mRNA expression in cells or tissues, (iv) expression or secretion of a downstream protein from a nucleic acid delivered such as the delivered nucleic acid in cells/tissues with overexpression of the target cell surface receptor as compared to normal cells/tissues or cells/tissues with low expression, (v) cytotoxicity.

In one embodiment, said target cells include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells. Thus, in one embodiment, the target cell is a cell in the liver. In one embodiment, the target cell is an epithelial cell. In one embodiment, the target cell is a hepatocyte. In one embodiment, the target cell is a hematopoietic cell. In one embodiment, the target cell is a muscle cell. In one embodiment, the target cell is an endothelial cell. In one embodiment the target cell is a tumor cell or a cell in the tumor microenvironment. In one embodiment, the target cell is a blood cell. In one embodiment, the target cell is a cell in the lymph nodes. In one embodiment, the target cell is a cell in the lung. In one embodiment, the target cell is a cell in the skin. In one embodiment, the target cell is a spleen cell. In one embodiment, the target cell is an antigen presenting cell such as a professional antigen presenting cell in the spleen. In one embodiment, the target cell is a dendritic cell in the spleen. In one embodiment, the target cell is a T cell. In one embodiment, the target cell is a B cell. In one embodiment, the target cell is a NK cell. In one embodiment, the target cell is a monocyte.

In some embodiments, said targeting fragment selectively or preferentially interacts with a particular cell type. The targeting fragment not only serves to selectively target the conjugates and polyplexes of present invention to a certain cell, but further typically facilitates selective uptake of the conjugates and corresponding polyplexes of the present invention within a certain cell type. In some embodiments, said targeting fragment selectively or preferentially interacts with a particular cell surface receptor. When the targeting fragment of a conjugate and/or polyplex selectively or preferentially interacts with a cell surface receptor, the conjugate and/or polyplex can be selectively or preferentially taken up into the cell that comprises said cell surface receptor.

In a preferred embodiment, said targeting fragment is a peptide, a protein, a small molecule ligand, a saccharide, an oligosaccharide, a lipid, an amino acid, wherein said peptide, said protein, said small molecule ligand, said saccharide, said oligosaccharide, said lipid, said amino acid is selected from a hormone, a neurotransmitter, a cytokine, a growth factor, a cell adhesion molecule, or a nutrient, and wherein said targeting fragment is an antibody, an antibody fragment, an aptamer or an affibody.

The term “small molecule ligand” as used herein, and in particular with reference to the inventive targeting fragment relates to a chemical moiety that has a molecular weight of at least 75 g/mol, preferably of at least 100 g/mol, and further preferably of at least 200 g/mol and has, preferably, a molecular weight of less than about 2000 g/mol. In some embodiments, the small molecule has a molecular weight of less than about 1500 g/mol, more preferably less than about 1000 g/mol. In a further preferred embodiment, the small molecule has a molecular weight of less than about 800 g/mol, again more preferably less than about 500 g/mol. The term “small molecule ligand” as used herein, and in particular with reference to the inventive targeting fragment shall further preferably relates to such ligand capable of binding, preferably specifically binding, to a target cell, to a target cell receptor, or preferably to a target cell surface receptor. In a preferred embodiment, said small molecule ligand has a molecular weight of at least 75 g/mol, preferably of at least 100 g/mol, and further preferably of at least 200 g/mol and has, preferably, a molecular weight of less than about 2000 g/mol, preferably of less than about 1500 g/mol. In a preferred embodiment, said small molecule ligand has a molecular weight of at least 75 g/mol, preferably of at least 100 g/mol, and further preferably of at least 200 g/mol and has, preferably, a molecular weight of less than about 2000 g/mol, preferably of less than about 1500 g/mol, and wherein said small molecule ligand is capable of binding, preferably specifically binding, to a target cell surface receptor.

In some embodiments, the targeting fragment is a native, natural or modified ligand or a paralog thereof, or a non-native ligand such as an antibody, a single-chain variable fragment (scFv), or an antibody mimetic such as an affibody. In a preferred embodiment, the targeting fragment is a native, natural or modified cell surface antigen ligand or a paralog thereof, or a non-native cell surface antigen ligand such as an antibody, a single-chain variable fragment (scFv), or an antibody mimetic such as an affibody. In a preferred embodiment, the targeting fragment is a native, natural or modified cell surface receptor ligand or a paralog thereof, or a non-native cell surface receptor ligand such as an antibody, a single-chain variable fragment (scFv), or an antibody mimetic such as an affibody. In a preferred embodiment, the targeting fragment is a small molecule ligand, a peptide, a protein, an aptamer, a native, natural or modified ligand and/or a paralog thereof. In a preferred embodiment, the targeting fragment is a small molecule ligand, a peptide, a protein, an aptamer, a native, natural or modified cell surface antigen ligand and/or a paralog thereof, wherein said small molecule ligand has a molecular weight of at least 75 g/mol, preferably of at least 100 g/mol, and further preferably of at least 200 g/mol and has, preferably, a molecular weight of less than about 2000 g/mol, preferably of less than about 1500 g/mol. In a preferred embodiment, the targeting fragment is a small molecule ligand, a peptide, a protein, an aptamer, a native, natural or modified cell surface receptor ligand and/or a paralog thereof, wherein said small molecule ligand has a molecular weight of at least 75 g/mol, preferably of at least 100 g/mol, and further preferably of at least 200 g/mol and has, preferably, a molecular weight of less than about 2000 g/mol, preferably of less than about 1500 g/mol. In a preferred embodiment, the targeting fragment is a small molecule ligand, a peptide, a protein, an aptamer, a native, natural or modified ligand and/or a paralog thereof, an antibody, a single-chain variable fragment (scFv), or an antibody mimetic such as an affibody.

In a preferred embodiment, the targeting fragment is a small molecule ligand, a peptide, a protein, an aptamer, a native, natural or modified cell surface receptor ligand and/or a paralog thereof. In a preferred embodiment, the targeting fragment is a small molecule ligand, a peptide, a protein, an aptamer, a native, natural or modified ligand and/or a paralog thereof, and wherein said small molecule ligand, said peptide, said protein, said aptamer, said native, natural or modified ligand and/or said paralog thereof is capable of binding, preferably selectively binding, to a cell surface receptor. In a preferred embodiment, said targeting fragment is a small molecule ligand. In a preferred embodiment, said targeting fragment is a small molecule ligand, wherein said small molecule ligand is capable of binding, preferably selectively binding, to a cell surface receptor. In a preferred embodiment, said targeting fragment is a peptide. In a preferred embodiment, said targeting fragment is a peptide, wherein said peptide is capable of binding, preferably selectively binding, to a cell surface receptor. In a preferred embodiment, said targeting fragment is a protein. In a preferred embodiment, said targeting fragment is a protein, wherein said protein is capable of binding, preferably selectively binding, to a cell surface receptor. In a preferred embodiment, said targeting fragment is an aptamer. In a preferred embodiment, said targeting fragment is an aptamer, wherein said aptamer is capable of binding, preferably selectively binding, to a cell surface receptor. In a preferred embodiment, said targeting fragment is a native, natural or modified ligand and/or a paralog thereof, preferably a native, natural or modified cell surface receptor ligand and/or a paralog thereof. In a preferred embodiment, said targeting fragment is a native, natural or modified ligand and/or a paralog thereof, wherein said native, natural or modified ligand and/or said paralog thereof is capable of binding, preferably selectively binding, to a cell surface receptor. In a preferred embodiment, said targeting fragment is an antibody, a single-chain variable fragment (scFv), or an antibody mimetic such as an affibody. In a preferred embodiment, said targeting fragment is an antibody, a single-chain variable fragment (scFv), or an antibody mimetic such as an affibody, wherein said antibody, a single-chain variable fragment (scFv), or an antibody mimetic such as an affibody is capable of binding, preferably selectively binding, to a cell surface receptor.

In a preferred embodiment, the targeting fragment is a small molecule ligand, a peptide, a protein, an aptamer, an antibody, an antibody fragment, preferably a single-chain variable fragment (scFv), an antibody mimetic, preferably selected from an affibody, nanobody, diabody, designed ankyrin repeat protein (DARPin), a growth factor or a functional fragment thereof, preferably hEGF), a hormone or a functional fragment thereof, preferably insulin, a cytokine or a functional fragment thereof, an integrin, an interleukin or a functional fragment thereof, an enzyme, a nucleic acid, a fatty acid, a carbohydrate, mono-, oligo- or polysaccharides, a peptidoglycan, a glycopeptide, asialoorosomucoid, mannose-6-phosphate, mannose, Sialyl-Lewis^{x}, N-acetyllactosamine, galactose, lysosomotropic agents, and/or a nucleus localizing agents, preferably T-antigen, a tumor low pH insertion peptide (PHLIP), a p32 targeting peptide, preferably LyP-1 tumor homing peptide, insulin-like growth factor 1, vascular endothelial growth factor, platelet-derived growth factor, and/or a fibroblast growth factor.

In some embodiments the targeting fragment is a non-native ligand such as an antibody or an antibody fragment (e.g., a single-chain variable fragment (scFv), an antibody mimetic such as an affibody, nanobody, diabody, designed ankyrin repeat protein (DARPin), or other antibody variant). In some embodiment, the targeting fragment is a growth factor or a fragment, preferably a functional fragment, thereof (e.g., hEGF); a hormone or a fragment preferably a functional fragment, thereof (e.g., insulin), asialoorosomucoid, mannose-6-phosphate, mannose, Sialyl-Lewis^{x}, N-acetyllactosamine, galactose, lysosomotropic agents, and/or a nucleus localizing agents (e.g., T-antigen), a tumor low pH insertion peptide (PHLIP), a p32 targeting peptide such as LyP-1 tumor homing peptide, insulin-like growth factor 1, vascular endothelial growth factor, platelet-derived growth factor, and/or a fibroblast growth factor. Further non-limiting examples of targeting fragments include an enzyme, a nucleic acid, a fatty acid, a carbohydrate, mono-, oligo- or polysaccharides, a peptidoglycan, a glycopeptide.

In a preferred embodiment, said targeting fragment is a small molecule ligand, a peptide, a protein, an aptamer, an antibody, an antibody fragment, preferably a Fab, Fab′, F(ab′)2 or a scFv fragment, an antibody mimetic, preferably selected from an affibody, nanobody, diabody, designed ankyrin repeat protein (DARPin), a growth factor or a functional fragment thereof, preferably hEGF, a hormone or a functional fragment thereof, preferably insulin, a cytokine or a functional fragment thereof, an interleukin or a functional fragment thereof, an enzyme, a nucleic acid, a fatty acid, a carbohydrate, mono-, oligo- or polysaccharides, a peptidoglycan, a glycopeptide, asialoorosomucoid, mannose-6-phosphate, mannose, Sialyl-Lewis^{x}, N-acetyllactosamine, galactose, lysosomotropic agents, and/or a nucleus localizing agents, preferably T-antigen, a tumor low pH insertion peptide (PHLIP), a p32 targeting peptide, preferably LyP-1 tumor homing peptide, insulin-like growth factor 1, vascular endothelial growth factor, platelet-derived growth factor, and/or a fibroblast growth factor.

In some embodiments, said targeting fragment L is selected from hEGF; an anti-HER2 peptide, preferably an anti-HER2 antibody or affibody; DUPA; a folate receptor-targeting fragment, folic acid; a somatostatin receptor-targeting fragment, preferably somatostatin and/or octreotide; an integrin-targeting fragment, preferably an arginine-glycine-aspartic acid (RGD)-containing fragment; a low pH insertion peptide; an asialoglycoprotein receptor-targeting fragment, preferably asialoorosomucoid; an insulin-receptor targeting fragment, preferably insulin; a mannose-6-phosphate receptor targeting fragment, preferably mannose-6-phosphate; a mannose-receptor targeting fragment, preferably mannose; a Sialyl Lewis^{x} antigen targeting fragments, preferably E-selectin; a sigma-2 receptor agonist, preferably N,N-dimethyltryptamine (DMT), sphingolipid-derived amine, and/or steroid, more preferably progesterone; a p32-targeting ligand, preferably anti-p32 antibody or p32-binding LyP-1 tumor-homing peptide; a Trop-2 targeting fragment, preferably an anti-Trop-2 antibody and/or antibody fragment; insulin-like growth factor 1; vascular endothelial growth factor; platelet-derived growth factor; and fibroblast growth factor.

In some embodiments, said targeting fragment L is selected from a targeting fragment derived from hEGF; an anti-HER2 peptide, preferably an anti-HER2 antibody or affibody; DUPA; folic acid; a somatostatin receptor-targeting fragment, preferably somatostatin and/or octreotide; an integrin-targeting fragment, preferably an arginine-glycine-aspartic acid (RGD)-containing fragment; a low pH insertion peptide; asialoglycoprotein receptor-targeting fragment, preferably asialoorosomucoid; an insulin-receptor targeting fragment, preferably insulin; a mannose-6-phosphate receptor targeting fragment, preferably mannose-6-phosphate; a mannose-receptor targeting fragment, preferably mannose; a Sialyl Lewis^{x} antigen targeting fragments, preferably E-selectin; a sigma-2 receptor agonist, preferably N,N-dimethyltryptamine (DMT), sphingolipid-derived amine, and/or steroid, more preferably progesterone; a p32-targeting ligand, preferably anti-p32 antibody or p32-binding LyP-1 tumor-homing peptide; a Trop-2 targeting fragment, preferably an anti-Trop-2 antibody and/or antibody fragment; insulin-like growth factor 1; vascular endothelial growth factor; platelet-derived growth factor; and fibroblast growth factor.

In a preferred embodiment, said targeting fragment is selected from an EGFR targeting fragment; a PSMA targeting fragment; an anti-HER2 peptide, preferably an anti-HER2 antibody or affibody; folic acid; a somatostatin receptor-targeting fragment, preferably somatostatin and/or octreotide; an integrin-targeting fragment, preferably an arginine-glycine-aspartic acid (RGD)-containing fragment; a low pH insertion peptide; asialoglycoprotein receptor-targeting fragment, preferably asialoorosomucoid; an insulin-receptor targeting fragment, preferably insulin; a mannose-6-phosphate receptor targeting fragment, preferably mannose-6-phosphate; a mannose-receptor targeting fragment, preferably mannose; a Sialyl Lewis^{x} antigen targeting fragments, preferably E-selectin; a sigma-2 receptor agonist, preferably N,N-dimethyltryptamine (DMT), sphingolipid-derived amine, and/or steroid, more preferably progesterone; a p32-targeting ligand, preferably anti-p32 antibody or p32-binding LyP-1 tumor-homing peptide; a Trop-2 targeting fragment, preferably an anti-Trop-2 antibody and/or antibody fragment; insulin-like growth factor 1; vascular endothelial growth factor; platelet-derived growth factor; and fibroblast growth factor.

In a preferred embodiment, the targeting fragment is an epidermal growth factor such as human epidermal growth factor (hEGF), wherein typically and preferably said coupling to the rest of said conjugate is effected via an amino group of said hEGF. The hEGF can be selectively taken up by cells that have increased expression (e.g., overexpression) of human epidermal growth factor receptor (EGFR).

In a preferred embodiment, said targeting fragment is capable of binding to epidermal growth factor receptor (EGFR), which is also named herein as EGFR targeting fragment.

EGFR is a transmembrane glycoprotein that is a member of the protein kinase superfamily and a receptor for members of the epidermal growth factor family. EGFR is a cell surface protein that binds to epidermal growth factor, thus inducing receptor dimerization and tyrosine autophosphorylation leading to cell proliferation. In a preferred embodiment, said EGFR targeting fragment is capable of binding to epitopes on the extracellular domain of EGFR.

In a preferred embodiment, said targeting fragment is capable of binding to a cell EGFR expressing. In a preferred embodiment, said targeting fragment is capable of binding to a cell overexpressing EGFR. In one embodiment, said cell overexpressing EGFR means that the level of EGFR expressed in said cell of a certain tissue is elevated in comparison to the level of EGFR as measured in a normal healthy cell of the same type of tissue under analogous conditions. In one embodiment, said cell overexpressing EGFR refers to an increase in the level of EGFR in a cell relative to the level in the same cell or closely related non-malignant cell under normal physiological conditions. In one embodiment, said cell overexpressing EGFR relates to expression of EGFR that is at least 10-fold, further preferably at least 20-fold, as compared to the expression of EGFR in a normal cell or in a normal tissue.

In a preferred embodiment, said targeting fragment is capable of binding to a cell expressing or overexpressing EGFR. For example, EGFR is overexpressed in neoplastic tissue and cancer types, such as glioma and carcinoma or cancer of epithelial origin, including of head and neck, thyroid, breast, ovarian, colon, gastric colorectal, stomach small intestine, cervix, bladder, lung, nasopharyngeal and esophageal tissue, such as squamous cells (e.g., Gan et al., J Cell Mol Med. 2009 September; 13(9b): 3993-4001; Aratani et al., Anticancer Research June 2017, 37 (6) 3129-3135), in particular in glioma, non-small-cell-lung-carcinoma, breast cancer, glioblastoma, squamous cell carcinoma, e.g. head and neck squamous cell carcinoma, small intestinal, colorectal cancer, adenocarcinoma, ovary cancer, bladder cancer or prostate cancer, and metastases thereof.

EGFR expression and overexpression are detected preferably using a monoclonal antibody targeting EGFR, e.g. by immunohistochemical methods (as e.g. described in Kriegs et al., Nature, 2019, 9:13564; Prenzel et al., Endocr Relat Cancer 8, 11-31, 2001). A cut-off of 5% or more EGFR positive cells can be used to define EGFR expression in different types of tissues or cells. Thus, cells or tissue with <5% positive cells can be considered to be negative.

In a preferred embodiment, said targeting fragment is capable of specifically binding to EGFR. Typically, specific binding refers to a binding affinity or dissociation constant K_{D} of the targeting fragment in the range of between about 1×10⁻³ M and about 1×10⁻¹² M. In preferred embodiment, said targeting fragment is capable of specifically binding to EGFR, wherein typically and preferably said affinity or specific binding is measured by the dissociation constant (K_{D}) and said affinity or specific binding refers to a K_{D} of less than 10⁻³ M, preferably of less than 10⁻⁴ M, further preferably of less than 10⁻⁵ M, further preferably of less than 106 M, more preferably of less than 10⁻⁷ M and even more preferably of less than 108 M, and again further preferably of less than 10⁻⁹ M. In a preferred embodiment, said targeting fragment is capable of specifically binding to EGFR, wherein typically and preferably said affinity or specific binding is measured by the dissociation constant (K_{D}) and said specific binding refers to a K_{D} of less than 10⁻³ M, of less than 10⁻⁴ M, of less than 10⁻⁵ M, of less than 106 M, of less than 10⁻⁷ M, of less than 108 M, and of less than 10⁻⁹ M. To detect binding or the complex or measure affinity, molecules can be analyzed using a competition binding assay, typically and preferably such as Biacore 3000 instrument (Biacore Inc., Piscataway NJ; as described, for example, in Wei-Ting Kuo et al., PLoS One. 2015, 10(2): e0116610 or in US2017224620A1). Preferably, binding results in formation of a complex between the EGFR targeting fragment and EGFR, wherein the binding or complex can be detected.

In a preferred embodiment, said targeting fragment is an EGFR antibody, an EGFR affibody, an EGFR aptamer, an EGFR targeting peptide or an EGFR targeting tyrosine kinase inhibitor. In a preferred embodiment, said EGFR targeting fragment is an EGFR antibody, an EGFR affibody, an EGFR aptamer, an EGFR targeting peptide or an EGFR targeting tyrosine kinase inhibitor.

In a preferred embodiment, said targeting fragment is an EGFR targeting peptide. An EGFR targeting peptide refers, typically and preferably, to peptide ligands of EGFR. Such peptide ligands are known to the skilled person and have been described, for example in US2017224620A1 and by Gent et al., 2018, Pharmaceutics 2018, 10, 2 (the disclosures of which are incorporated herein by reference in its entirety). EGFR targeting peptides have low immunogenic potential and show good penetration into solid tumor tissues.

In a preferred embodiment, said EGFR targeting peptide has a molecular weight of about 1000 g/mol to about 2000 g/mol, preferably of about 1100 g/mol to about 1900 g/mol, further preferably of about 1200 g/mol to about 1800 g/mol, and again more preferably of about 1300 g/mol to about 1700 g/mol.

In a preferred embodiment, the EGFR targeting peptide comprises, or preferably consists of, the sequence YHWYGYTPQNVI (GE11) (SEQ ID NO:9). In a preferred embodiment, said targeting fragment comprises, or preferably consists of, the sequence YHWYGYTPQNVI (GE 11) (SEQ ID NO:9). GE-11 has excellent affinity towards EGFR and shows also binding specificity for EGFR (kd=22 nM) (Ruoslahti et al., Adv. Mater. 2012, 24, 3747-3756; Li et al., J. Res. Commun. 2005, 19, 1978-1985). GE11 moves from EGFR after the addition of the physiologic ligand EGF, demonstrating both its selective binding to EGFR and its receptor affinity. GE11 has been reported to have a high potential to accelerate nanoparticle endocytosis due to an alternative EGFR-dependent actin-driven pathway. (Mickeler et al., Nano Lett. 2012, 12, 3417-3423; Song et al., FASEB J. 2009, 23, 1396-1404) It has been showed that the EGFR level on the surface of cancer cells remains constant after treatment with GE11 polyplexes, indicating an EGFR recycling process with a prolonged receptivity of the cells for circulating GE11 polyplexes.

In a preferred embodiment, said EGFR targeting fragment comprises, or preferably consists of, GE11 (SEQ ID NO:9), in particular, in use for treating solid tumors characterized by EGFR-overexpressing cells. The inventive conjugate and polyplexes comprising, or preferably consisting, GE11 as the targeting fragment are believed to be stable polyplexes ensuring that the polyanion and nucleic acid payload is not released before the polyplex has reached its target cell.

In a preferred embodiment, said targeting fragment is an EGFR antibody. An EGFR antibody refers to an antibody that binds to EGFR. In a preferred embodiment, said EGFR antibody is a human. In a preferred embodiment, said EGFR antibody is a humanized EGFR antibody. In a preferred embodiment, said EGFR antibody is a monoclonal human. In a preferred embodiment, said EGFR antibody is a humanized EGFR antibody. In a preferred embodiment, said EGFR antibody is a monoclonal fully human EGFR antibody. In another preferred embodiment, the EGFR antibody is a scFv or Fab fragment.

EGFR antibodies are known to the skilled person and have been described for example in WO2008/105773 and in WO2017/185662 (the disclosure of which is incorporated herein by reference in its entirety) and include Bevacizumab, Panitumumab, Cetuximab, Tomuzotuximab, Futuximab, Zatuximab, Modotuximab, Imgatuzumab, Zalutumumab, Matuzumab, Necitumumab, Nimotuzumab, CEVIAvax EGF, clones EGFR, L8A4, E6.2, TH190DS, Pep2, Pep3, LR-DM1, PlX, YC088, ratML66, FM329, TGM10-1, F4, 2F8, 15H8, TAB-301MZ—S(P), mAb528, 2224, E7.6.3, C225, CBL155, MR1, MR1, L211C, N5-4, TH83DS, L2-12B, 15H8, 12Do3, 7A7, 42C11 (MOB-1078z), PABL-080, HPAB-2204LY—S(P), VHH205, ABT-806, Tab-271MZ, Hu225, LA22, Fab fragment DL 11, Fab fragment DX 1-6, VHH104, OA-cb6, 07D06, Fab fragment HPAB-0419-FY—F(E), Fab fragment TAB-285MZ-F(E), Fab fragment TAB-293MZ-F(E), Fab fragment HPAB-0136-YJ-F(E), FGF-R2, EG-19-11, Fab fragment pSEX81-63, DX 1-4, scFv fragment DX 1-6, EG-26-11, EG-26-11, DX1-4, TAB-326MZ, scFv fragment 528, scFv fragment LA1, scFv fragment 07D06, single domain antibody VHH139, scFv fragment EG-19-11, single domain Antibody VHH134, single domain Antibody 9G8, ABT-414, AMG-595, and IMGN-289. One of ordinary skill in the art will appreciate that any antibody that recognizes and/or specifically binds to EGFR may be used in accordance with the present invention.

In a preferred embodiment, said targeting fragment is an EGFR inhibitor. An EGFR inhibitor refers to targeting fragment that block cell-surface localization and signaling of the EGFR, such as oligosaccharyltransferase inhibitors like nerve growth inhibitor-1; or EGFR kinase inhibitors, such as afatinib, erlotinib, osimertinib and gefitinib. EGFR inhibitors are known to the skilled person and have been described for example in WO2018078076 and in US2017224620A1 (the disclosure of which is incorporated herein by reference in its entirety).

In a preferred embodiment, said targeting fragment is an EGFR aptamer. Preferred EGFR targeting aptamers include, but are not limited to those disclosed in Na Li et al. (PLoS One. 2011; 6(6): e20299), Deng-LiangWang et al. (Biochemical and Biophysical Res Com, 453(4), 2014, pp 681-685), Min Woo Kim et al. (Theranostics 2019; 9(3):837-852), Akihiro Eguchi et al. (JACS Au 2021, 1, 5, 578-585) or Yingpan Song et al. (RSC Adv., 2020, 10, 28355-28364), the disclosures of which are incorporated herein by reference in its entirety.

The term EGFR aptamer includes also EGFR aptamer derivatives and/or functional fragments of EGFR aptamer. In some embodiments, in the EGFR aptamer derivatives fewer than 30, 25, 20, 15, 10, 5, 4, 3, 2, or 1 nucleic acid is substituted relative to the corresponding EGFR aptamer. In some embodiments, the sequences of the EGFR aptamer derivatives are at least 80%, preferably 85%, more preferably 90%, again more preferably 95%, most preferably 99% identical with the corresponding EGFR aptamer.

In a preferred embodiment, said targeting fragment is an EGFR affibody. Preferred EGFR affibodies include, but are not limited to ZEGFR:1907, ZEGFR:2377 or ZEGFR:03115 (available from Affibody Medical AB) or the dimeric form of these affibodies. In a preferred embodiment said EGFR affibody has the sequence of SEQ ID NO:8.

In a preferred embodiment, said targeting fragment is the EGFR ligand epidermal growth factor (EGF). Thus, in a preferred said targeting fragment is epidermal growth factor (EGF). In a preferred embodiment, said targeting fragment is human EGF (hEGF), mouse EGF (mEGF), rat EGF, or guinea pig EGF. In a very preferred embodiment, said targeting fragment is human EGF (hEGF). In a very preferred embodiment, said targeting fragment comprises, preferably consists of, the sequence of SEQ ID NO:7.

In some embodiments, EGF is modified, e.g., by deleting or exchanging one or more amino acids or truncation of EGF. Modified and/or truncated EGF molecules are for example disclosed in WO2019023295A1. EGF has many residues conserved across rat, mouse, guinea pig and human species (Savage et al., J. Biol. Chem.., 247: 7612-7621, 1973; Carpenter and Cohen, Ann. Rev. Biochem., 48: 193-316, 1979; Simpson et al., Eur J Biochem, 153:629-37, 1985). In particular, six cysteine residues at positions 6, 14, 20, 31, 33, and 42 are conserved as they form three disulfide bridges to provide conserved tertiary protein structure. Also conserved across all four species are residues as positions 7, 9, 11, 12, 13, 15, 18, 21, 24, 29, 32, 34, 36, 37, 39, 41, 46, and 47. Many of these residues may be expected to facilitate or provide key binding interactions with the corresponding EGFR. It has been described that both the full length human EGF (53 residues) and a truncated form (48 residues), which results from trypsin cleavage, retain strong binding affinity and activation of the EGFR (Calnan et al., 47(5):622-7, 2000; Gregory, Regul Pept, 22:217-26, 1988). Mutagenesis studies have been reported for various residues to correlate the effect of replacement of specific residues on binding of EGF to the EGFR or activation of the EGFR (Campion et al., Biochemistry, 29, 9988-9993, 1990; Engler et al., J. Biol. Chem., 267:2274-2281, 1992; Tadaki and Niyogi. J. Biol. Chem., 268: 10114-10119, 1993). An x-ray crystal structure of EGF bound to EGFR has been solved which shows key binding interactions and also identifies residues not directly involved in binding (Ogiso et al., Cell, Vol. 110, 775-787, 2002).

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

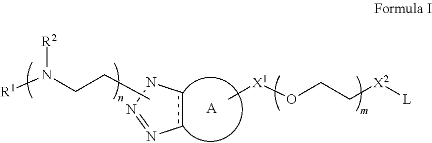

wherein:
[symbol] is a single bond or a double bond;
n is any integer between 1 and 1500, preferably any integer between 2 and 1500;
m is a discrete number of repeating —(O—CH₂—CH₂)—units, wherein said discrete number m of repeating —(O—CH₂—CH₂)—units is any discrete number of 25 to 100, preferably of 25 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a polyplex, wherein said polyplex comprise a conjugate of the Formula I, or a pharmaceutically acceptable salt, solvate, hydrate, tautomer or enantiomer thereof:

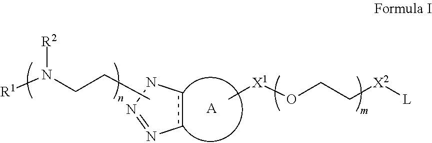

wherein:
[symbol] is a single bond or a double bond;
n is any integer between 1 and 1500, preferably any integer between 2 and 1500;
m is a discrete number of repeating —(O—CH₂—CH₂)—units, wherein said discrete number m of repeating —(O—CH₂—CH₂)—units is any discrete number of 25 to 100, preferably of 25 to 60, and wherein further preferably said discrete number m of repeating —(O—CH₂—CH₂)—units is 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a composition comprising a polyplex, wherein said polyplex comprise a conjugate, preferably a plurality of conjugates, of the Formula I, or a pharmaceutically acceptable salt, solvate, hydrate, tautomer or enantiomer thereof, and a nucleic acid, wherein said nucleic acid is preferably non-covalently bound to said conjugate:

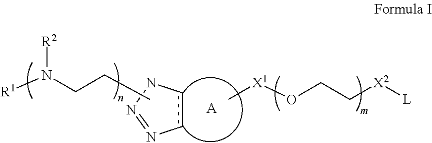

wherein:
[symbol] is a single bond or a double bond;
n is any integer between 1 and 1500, preferably any integer between 2 and 1500;
m a discrete number of repeating —(O—CH₂—CH₂)—units, wherein said discrete number m of repeating —(O—CH₂—CH₂)—units is 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

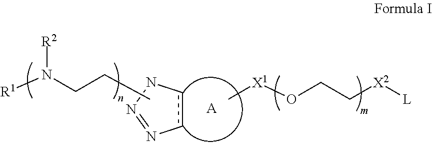

wherein.
[symbol] is a single bond or a double bond;
n is any integer between 1 and 1500, preferably any integer between 2 and 1500;
m is a discrete number of repeating —(O—CH₂—CH₂)—units, wherein said discrete number m of repeating —(O—CH₂—CH₂)—units is 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a composition comprising a polyplex, wherein said polyplex comprise a conjugate, preferably a plurality of conjugates, of the Formula I, or a pharmaceutically acceptable salt, solvate, hydrate, tautomer or enantiomer thereof, and a nucleic acid, wherein said nucleic acid is preferably non-covalently bound to said conjugate:

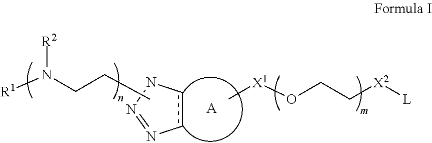

wherein:
[symbol] is a single bond or a double bond;
n is any integer between 1 and 1500, preferably any integer between 2 and 1500;
m is a discrete number of contiguous repeating —(O—CH₂—CH₂)—units m of 25 to 100, preferably of a discrete number of contiguous repeating —(O—CH₂—CH₂)—units m of 25 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

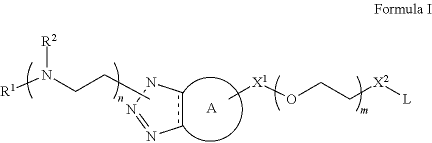

wherein:
[symbol] is a single bond or a double bond;
n is any integer between 1 and 1500, preferably any integer between 2 and 1500;
m is a discrete number of contiguous repeating —(O—CH₂—CH₂)—units m of 25 to 100, preferably of a discrete number of contiguous repeating —(O—CH₂—CH₂)—units m of 25 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a composition comprising a polyplex, wherein said polyplex comprise a conjugate, preferably a plurality of conjugates, of the Formula I, or a pharmaceutically acceptable salt, solvate, hydrate, tautomer or enantiomer thereof, and a nucleic acid, wherein said nucleic acid is preferably non-covalently bound to said conjugate:

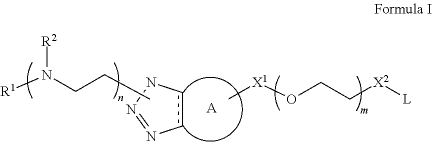

wherein:
[symbol] is a single bond or a double bond;
n is any integer between 1 and 1500, preferably any integer between 2 and 1500;
m is a discrete number of contiguous repeating —(O—CH₂—CH₂)—units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

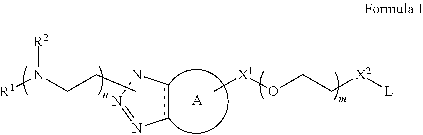

wherein:
[symbol] is a single bond or a double bond;
n is any integer between 1 and 1500, preferably any integer between 2 and 1500;
m is a discrete number of contiguous repeating —(O—CH₂—CH₂)—units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is capable of binding to epidermal growth factor receptor (EGFR), and wherein preferably said targeting fragment is capable of binding to a cell expressing EGFR, and wherein further preferably said targeting fragment is capable of binding to a cell surface receptor, wherein said cell surface receptor is EGFR, and
wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃.

In another aspect, the present invention provides a composition comprising a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

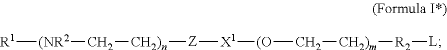

wherein
n is any integer between 1 and 1500, preferably any integer between 2 and 1500;
m is any integer between 1 and 200, preferably m is any integer between 1 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ—is H;
X¹ and X² are independently divalent covalent linking moieties;
Z is a divalent covalent linking moiety wherein Z is not a single bond and Z is not —NHC(O)—;
L is a targeting fragment, wherein said targeting fragment comprises, or preferably consists of, the sequence YHWYGYTPQNVI (GE 11) (SEQ ID NO:9), and
wherein said nucleic acid is a pharmaceutically active nucleic acid, wherein said pharmaceutically active nucleic acid is a nucleic acid that encodes a pharmaceutically active peptide or protein, and
wherein preferably said composition consists of said conjugate.

In another aspect, the present invention provides a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

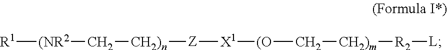

wherein
n is any integer between 1 and 1500, preferably any integer between 2 and 1500;
m is any integer between 1 and 200, preferably m is any integer between 1 and 100;
R¹ is an initiation residue, wherein preferably R¹ is —H or —CH₃;
R² is independently —H or an organic residue, wherein at least 80%, preferably 90% of said R² in said —(NR²—CH₂—CH₂)ₙ—is H;
X¹ and X² are independently divalent covalent linking moieties;
Z is a divalent covalent linking moiety wherein Z is not a single bond and Z is not —NHC(O)—;
L is a targeting fragment, wherein said targeting fragment comprises, or preferably consists of, the sequence YHWYGYTPQNVI (GE 11) (SEQ ID NO:9), and
wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

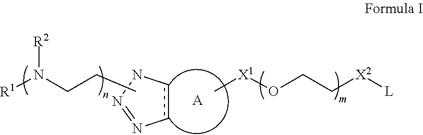

wherein:
[symbol] is a single bond or a double bond;
n is any integer between 1 and 1500, preferably any integer between 2 and 1500;
m is any integer between 1 and 200;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment comprises, or preferably consists of, the sequence YHWYGYTPQNVI (GE 11) (SEQ ID NO:9), and
wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

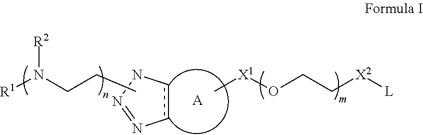

wherein:
[symbol] is a single bond or a double bond;
n is any integer between 1 and 1500, preferably any integer between 2 and 1500;
m is any integer between 1 and 200;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment comprises, or preferably consists of, the sequence YHWYGYTPQNVI (GE 11) (SEQ ID NO:9), and wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

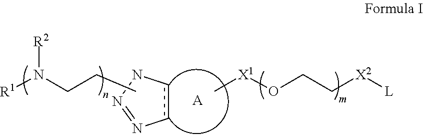

wherein:
[symbol] is a single bond or a double bond;
n is any integer between 1 and 1500, preferably any integer between 2 and 1500;
m is a discrete number of repeating units m of 2 to 100, preferably of a discrete number of repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an EGFR targeting fragment, wherein preferably said EGFR targeting fragment is capable of specifically binding to a cell expressing, preferably overexpressing, EGFR, and
wherein said targeting fragment is epidermal growth factor (EGF), and wherein preferably said targeting fragment is human EGF (hEGF), and wherein again further preferably said targeting fragment comprises, preferably consists of, the sequence of SEQ ID NO:7, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

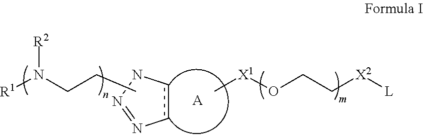

wherein:
[symbol] is a single bond or a double bond;
n is any integer between 1 and 1500, preferably any integer between 2 and 1500;
m is a discrete number of contiguous repeating units m of 2 to 100, preferably of a discrete number of contiguous repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an EGFR targeting fragment, wherein preferably said EGFR targeting fragment is capable of specifically binding to a cell expressing, preferably overexpressing, EGFR, and
wherein said targeting fragment is epidermal growth factor (EGF), and wherein preferably said targeting fragment is human EGF (hEGF), and wherein again further preferably said targeting fragment comprises, preferably consists of, the sequence of SEQ ID NO:7, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

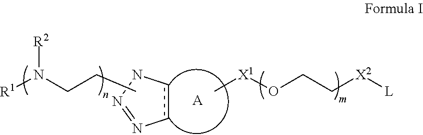

wherein:
[symbol] is a single bond or a double bond;
n is any integer between 1 and 1500, preferably any integer between 2 and 1500;
m is a discrete number of repeating units m of 2 to 100, preferably of a discrete number of repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

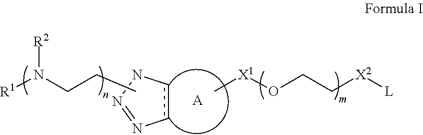

wherein:
[symbol] is a single bond or a double bond;
n is any integer between 1 and 1500, preferably any integer between 2 and 1500;
m is a discrete number of contiguous repeating units m of 2 to 100, preferably of a discrete number of contiguous repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor, and
wherein said nucleic acid is a pharmaceutically active nucleic acid, wherein said pharmaceutically active nucleic acid is a nucleic acid that encodes a pharmaceutically active peptide or protein.

In a preferred embodiment, said targeting fragment is capable of binding to prostate specific membrane antigen (PSMA), which is also named herein as PSMA targeting fragment.

PSMA is a multifunctional transmembrane protein that functions as a glutamate carboxypeptidase and also demonstrates rapid, ligand-induced internalization and recycling (Liu H, et al., 1998, Cancer Res 58:4055-4060). PSMA is mainly expressed in four tissues of the body, including prostate epithelium, the proximal tubules of the kidney, the jejunal brush border of the small intestine and ganglia of the nervous system (Mhawech-Fauceglia et al., Histopathology 2007, 50:472-483). In a preferred embodiment, said targeting fragment is capable of binding to epitopes on the extracellular domain of PSMA.

In a preferred embodiment, said targeting fragment, preferably said PSMA targeting fragment, is capable of binding to a cell expressing PSMA. In a preferred embodiment, said targeting fragment, preferably said PSMA targeting fragment, is capable of binding to a cell overexpressing PSMA. For example, PSMA is overexpressed in neoplastic tissue and in malignant prostate, especially in prostatic adenocarcinoma relative to normal tissue, and the level of PSMA expression is further up-regulated as the disease progresses into metastatic phases (Silver et al., 1997, Clin. Cancer Res., 3:81). PSMA is expressed and overexpressed also in other tumor types (Mhawech-Fauceglia et al., Histopathology 2007, 50:472-483; Israeli R S et al, Cancer Res 1994, 54:1807-1811; Chang S S et al, Cancer Res 1999, 59:3192-198).

In one embodiment, said overexpressing PSMA means that the level of PSMA expressed in said cell of a certain tissue is elevated in comparison to the level of PSMA as measured in a normal healthy cell of the same type of tissue under analogous conditions. In one embodiment, said overexpressing PSMA refers to an increase in the level of PSMA in a cell relative to the level in the same cell or closely related non-malignant cell under normal physiological conditions. In one embodiment, said cell overexpressing PSMA relates to expression of PSMA that is at least 10-fold higher as compared to a normal cell or a normal tissue. In one embodiment, said cell overexpressing PSMA relates to expression of PSMA with a cut-off of 5% or more PSMA positive cells, as e.g. described in Mhawech-Fauceglia et al., 2007, which can be used to define PSMA expression in different types of tissues or cells. Thus, cells or tissue with <5% positive cells was considered to be negative, or where the PSMA expression is categorized according to its intensity and scored as 0 (no expression), 1 (low expression), 2 (medium expression), and 3 (high expression), as described in Hupe et al., 2018 2018 (Hupe M C et al, Frontiers in Oncology 2018, 8 (623): 1-7).

In a preferred embodiment, said targeting fragment is capable of binding to a cell expressing or overexpressing PSMA. Cells expressing PSMA typically include tumor cells, such as prostate, bladder, pancreas, lung, kidney, colon tumor cells, melanomas, and sarcomas. In a preferred embodiment said targeting fragment is capable of binding to a cell expressing or overexpressing PSMA, wherein said cell is a tumor cell, preferably selected from a prostate, a bladder, a pancreas, a lung, a kidney and a colon tumor cell, a melanoma, and a sarcoma. In a preferred embodiment said targeting fragment is capable of binding to a cell expressing or overexpressing PSMA, wherein said cell is a tumor cell, wherein said tumor cell is a prostate tumor cell.

In a preferred embodiment, said targeting fragment is capable of specifically binding to PSMA, wherein typically and preferably said affinity or specific binding is measured by the dissociation constant (K_{D}) and said affinity or specific binding refers to a K_{D} of less than 10⁻³ M, preferably of less than 10⁻⁴ M, further preferably of less than 10⁻⁵ M, further preferably of less than 10⁻⁶ M, more preferably of less than 10⁻⁷ M and even more preferably of less than 10⁻⁸ M, and again further preferably of less than 10⁻⁹ M, and again further preferably of less than 10⁻¹⁰ M. In a preferred embodiment, said targeting fragment is capable of specifically binding to PSMA, wherein typically and preferably said affinity or specific binding is measured by the dissociation constant (K_{D}) and said affinity or specific binding refers to a K_{D} of less than 10⁻³ M, of less than 10⁻⁴ M, of less than 10⁻⁵ M, of less than 10⁻⁶ M, of less than 10⁻⁷ M, of less than 10⁻⁸ M, and of less than 10⁻⁹ M. Preferably, binding results in formation of a complex between the targeting fragment and PSMA, wherein the binding or complex can be detected, typically and preferably using a Biacore 3000 instrument (Biacore Inc., Piscataway NJ) or cell based binding assays or Flow Induced Dispersion Analysis (FIDA), typically and preferably as described in Kularatne et al, Mol Pharm. 2009; 6(3): 790-800.

In a preferred embodiment, said targeting fragment is a PSMA antibody, a PSMA aptamer or a small-molecule PSMA targeting fragment. In a preferred embodiment, said PSMA targeting fragment is a PSMA antibody, a PSMA aptamer or a small-molecule PSMA targeting fragment. The term “small molecule PSMA targeting fragment” as used herein relates to a chemical moiety that has a molecular weight of less than about 2000 g/mol, and that is typically and preferably capable of binding to PSMA. In some embodiments, the small molecule PSMA targeting fragment has a molecular weight of less than about 1800 g/mol. In some embodiments, the small molecule PSMA targeting fragment has a molecular weight of less than about 1500 g/mol, more preferably less than about 1000 g/mol. In a further preferred embodiment, the small molecule has a molecular weight of less than about 800 g/mol, again more preferably less than about 500 g/mol.

In some embodiments, said PSMA targeting fragment is a PSMA antibody that is an antibody capable of binding to PSMA. In some embodiments, said antibody is a monoclonal antibody, a polyclonal antibody, and/or an antibody fragment, preferably a functional fragment thereof, a chimeric antibody, a recombinant antibody, and/or a bi- or multispecific antibody. Such PSMA antibodies include, but are not limited to, scFv antibodies A5, G0, G1, G2, and G4 and mAbs 3/E7, 3/F11, 3/A12, K7, K12, and D20 (Elsasser-Beile et al., 2006, Prostate, 66:1359); mAbs E99, J591, J533, and J415 (Liu et al., 1997, Cancer Res., 57:3629; Liu et al., 1998, Cancer Res., 58:4055; Fracasso et al., 2002, Prostate, 53:9; McDevitt et al., 2000, Cancer Res., 60:6095; McDevitt et al., 2001, Science, 294:1537; Smith-Jones et al., 2000, Cancer Res., 60:5237; Vallabhajosula et al., 2004, Prostate, 58:145; Bander et al., 2003, J. Urol., 170:1717; Patri et al., 2004, Bioconj. Chem., 15:1174; and U.S. Pat. No. 7,163,680); mAb 7E11-C5.3 (Horoszewicz et al., 1987, Anticancer Res., 7:927); antibody 7E11 (Horoszewicz et al., 1987, Anticancer Res., 7:927; and U.S. Pat. No. 5,162,504); and antibodies described in Chang et al., 1999, Cancer Res., 59:3192; Murphy et al., 1998, J. Urol., 160:2396; Grauer et al., 1998, Cancer Res., 58:4787; and Wang et al., 2001, Int. J. Cancer, 92:871. One of ordinary skill in the art will appreciate that any antibody that recognizes and/or specifically binds to PSMA may be used in accordance with the present invention. All foregoing documents and disclosures are incorporated herein by reference in their entirety.

In some embodiments, said targeting fragment capable of binding to PSMA is an aptamer. PSMA targeting aptamers include, but are not limited to, the A10 aptamer or A9 aptamer (Lupold et al., 2002, Cancer Res., 62:4029; and Chu et al., 2006, Nuc. Acid Res., 34: e73), derivatives thereof, and/or functional fragments thereof. In some embodiments, in the aptamer derivatives fewer than 30, 25, 20, 15, 10, 5, 4, 3, 2, or 1 nucleic acid is substituted relative to the aptamer. In some embodiments, the sequences of the aptamer derivatives are at least 80%, preferably 85%, more preferably 90%, again more preferably 95%, most preferably 99% identical.

In a preferred embodiment, said targeting fragment is a small molecule PSMA targeting fragment. In a preferred embodiment, said PSMA targeting fragment is a small molecule PSMA targeting fragment, preferably a small molecule PSMA targeting peptidase inhibitor. In a preferred embodiment, said small molecule PSMA peptidase inhibitors include 2-PMPA, GPI5232, VA-033, phenylalkylphosphonamidates (Jackson et al., 2001, Curr. Med. Chem., 8:949; Bennett et al., 1998, J. Am. Chem. Soc., 120:12139; Jackson et al., 2001, J Med. Chem., 44:4170; Tsukamoto et al., 2002, Bioorg. Med. Chem. Lett., 12:2189; Tang et al., 2003, Biochem. Biophys. Res. Commun., 307: 8; Oliver et al., 2003, Bioorg. Med. Chem., 11:4455; and Maung et al., 2004, Bioorg. Med. Chem., 12:4969), and/or analogs and derivatives thereof. All of the foregoing documents (scientific and other publications, patents and patent applications) are incorporated herein by reference in their entirety. In some embodiments, said small molecule PSMA targeting fragment is a protein, a peptide, an amino acid or a derivative thereof. In a preferred embodiment, said small molecule PSMA targeting fragment includes thiol and indole thiol derivatives, such as 2-MPPA and 3-(2-mercaptoethyl)-1H-indole-2-carboxylic acid derivatives (Majer et al., 2003, J Med. Chem., 4611989; and U.S. Patent Publication 2005/0080128). In some embodiments, said small molecule PSMA targeting fragments comprise hydroxamate derivatives (Stoermer et al., 2003, Bioorg. Med. Chem. Lett., 1312097). In a preferred embodiment, said small molecule PSMA peptidase inhibitors include androgen receptor targeting agents (ARTAs), such as those described in U.S. Pat. Nos. 7,026,500; 7,022,870; 6,998,500; 6,995,284; 6,838,484; 6,569,896; 6,492,554; and in U.S. Patent Publications 2006/0287547; 2006/0276540; 2006/0258628; 2006/0241180; 2006/0183931; 2006/0035966; 2006/0009529; 2006/0004042; 2005/0033074; 2004/0260108; 2004/0260092; 2004/0167103; 2004/0147550; 2004/0147489; 2004/0087810; 2004/0067979; 2004/0052727; 2004/0029913; 2004/0014975; 2003/0232792; 2003/0232013; 2003/0225040; 2003/0162761; 2004/0087810; 2003/0022868; 2002/0173495; 2002/0099096; 2002/0099036. In some embodiments, said small molecule PSMA targeting fragments include polyamines, such as putrescine, spermine, and spermidine (U.S. Patent Publications 2005/0233948 and 2003/0035804). All foregoing documents and disclosures are incorporated herein by reference in their entirety.

In a preferred embodiment, said small molecule PSMA peptidase inhibitors include PBDA- and urea-based inhibitors, such as ZJ 43, ZJ, ZJ 17, ZJ 38 (Nan et al., 2000, J. Med. Chem., 43:772; and Kozikowski et al., 2004, J. Med. Chem., 47, 7, 1729-1738), and/or and analogs and derivatives thereof. Other agents which bind PSMA can also be used as PSMA targeting fragment including, for example those found in Clin. Cancer Res., 2008 14:3036-43, or PSMA targeting fragments prepared by sequentially adding components to a preformed urea, such as the lysine-urea-glutamate compounds described in Banerjee et al. (J. Med. Chem. vol. 51, pp. 4504-4517, 2008). In a preferred embodiment, said one or more targeting fragments capable of binding to prostate specific membrane antigen (PSMA) are small-molecule PSMA targeting fragments, more preferably small urea-based inhibitors.

In preferred embodiments, said small molecule PSMA targeting fragments are urea-based inhibitors (herein also called urea-based peptidase inhibitors), more preferably small urea-based inhibitors, such as disclosed in Kularatne et al., Mol Pharmaceutics 2009, 6, 780; Kularatne et al., Mol. Pharmaceutics 2009, 6, 790; Kopka et al., J Nucl Med 2017, 58:17S-26S, Kozikowski et al., J Med Chem. 2001, 44:298-301, Kozikowski et al., J Med Chem. 2004, 47:1729-1738, WO2017/044936, WO2011/084518, WO2011/084521, WO2011/084513, WO2012/166923, WO2008/105773, WO2008/121949, WO2012/135592, WO2010/005740, WO2015/168379, WO03/045436, WO03/045436, WO2016/183447, US2015/258102, WO2011/084513, WO 2017/089942, US2010/278927, WO2012/016188, WO2008/124634, WO2009/131435, US 2007/225213, WO2017/086467, WO2009/026177, WO2012005572, WO2014/072357, and WO2011/108930. All foregoing documents and disclosures are incorporated herein by reference in their entirety.

In a preferred embodiment, said targeting fragment is a dipeptide urea based PSMA peptidase inhibitor, preferably a small molecule dipeptide urea-based PSMA peptidase inhibitor. In a preferred embodiment, said PSMA targeting fragment is a dipeptide urea based PSMA peptidase inhibitor, preferably a small molecule dipeptide urea-based PSMA peptidase inhibitor.

The term “urea based PSMA peptidase inhibitor” relate to a PSMA peptidase inhibitor comprising an urea group. The term “dipeptide urea based PSMA peptidase inhibitor” relate to PSMA peptidase inhibitor comprising an urea group and two peptides or amino acids each independently attached to the —NH₂ groups of the urea group, while the term “small molecule dipeptide urea-based PSMA peptidase inhibitor” further refers that the dipeptide urea based PSMA peptidase inhibitor has a molecular weight of less than about 2000 g/mol, and that is typically and preferably capable of binding to PSMA. In some embodiments, the small molecule dipeptide urea-based PSMA peptidase inhibitor has a molecular weight of less than about 1800 g/mol, less than about 1500 g/mol, preferably less than about 1000 g/mol. In a further preferred embodiment, the small molecule dipeptide urea-based PSMA peptidase inhibitor has a molecular weight of less than about 800 g/mol, again more preferably less than about 500 g/mol. PSMA peptidase inhibitors are able to reduce the activity of the PSMA transmembrane zinc(II) metalloenzyme that catalyzes the cleavage of terminal glutamates. More preferably, said small molecule urea-based PSMA peptidase inhibitor has a molecular weight of less than about 500 g/mol. Again more preferably, said small molecule urea-based PSMA peptidase inhibitor is a Glutamate-urea based PSMA peptidase inhibitor, preferably such as mentioned in Kopka et al., J Nuc Med, 58(9), suppl. 2, 2017; Wirtz et al., EJNMMI Research (2018) 8:84 and references cited therein, all incorporated herein by reference in their entirety.

In a preferred embodiment, said targeting fragment, preferably said urea based PSMA peptidase inhibitor is a glutamate-urea moiety of formula 1, preferably of formula 1*:

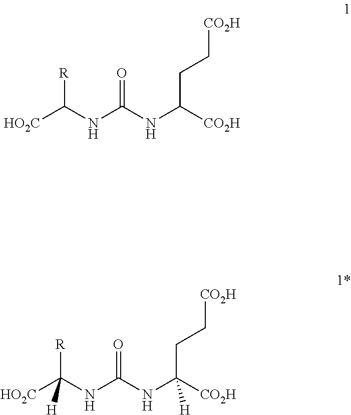

and enantiomers, stereoisomers, rotamers, tautomers, diastereomers, or racemates thereof; wherein R is preferably substituted or unsubstituted alkyl, substituted or unsubstituted aryl, and any combination thereof, more preferably R is C₁₋₆-alkyl, preferably C₂-C₄-alkyl, substituted one or more times, preferably one time with OH, SH, NH₂, or COOH, wherein one of said NH₂, OH or SH or COOH group serve as the point of covalent attachment to the X² linking moiety and the PEG fragment respectively, wherein the alkyl group is optionally be interrupted by N(H), S or O. In another preferred embodiment, R is C₁₋₆-alkyl, preferably C₂-C₄-alkyl, substituted one time with OH, SH, NH₂, or COOH, wherein said NH₂, OH, or SH or COOH group serve as the point of covalent attachment to the X² linking moiety and the PEG fragment respectively. In a very preferred embodiment, R is C₂-alkyl substituted one time with COOH, wherein said COOH group serve as the point of covalent attachment to the X² linking moiety and the PEG fragment respectively.

In a preferred embodiment, said targeting fragment is a glutamate-urea moiety of formula 1:

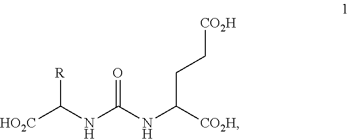

wherein R is C₁₋₆-alkyl, preferably C₂-C₄-alkyl, substituted one or more times, preferably one time with OH, SH, NH₂, or COOH, wherein one of said NH₂, OH or SH or COOH group serve as the point for covalent attachment to the X² linking moiety and the PEG fragment respectively, and wherein the alkyl group is optionally be interrupted by N(H), S or O. In another preferred embodiment, R is C₁₋₆-alkyl, preferably C₂-C₄-alkyl, substituted one time with OH, SH, NH₂, or COOH, wherein said NH₂, OH, or SH or COOH group serve as the point for covalent attachment to the X² linking moiety and the PEG fragment respectively. In a very preferred embodiment, R is C₂-alkyl substituted one time with COOH, wherein said COOH group serve as the point for covalent attachment to the X² linking moiety and the PEG fragment respectively.

In another preferred embodiment, said targeting fragment is a glutamate-urea moiety of formula 1*

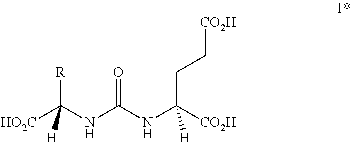

wherein R is C₁₋₆-alkyl, preferably C₂-C₄-alkyl, substituted one or more times, preferably one time with OH, SH, NH₂, or COOH, wherein one of said NH₂, OH or SH or COOH group serve as the point for covalent attachment to the X² linking moiety and the PEG fragment respectively, and wherein the alkyl group is optionally be interrupted by N(H), S or O. In another preferred embodiment, R is C₁₋₆-alkyl, preferably C₂-C₄-alkyl, substituted one time with OH, SH, NH₂, or COOH, wherein said NH₂, OH, or SH or COOH group serve as the point for covalent attachment to the X² linking moiety and the PEG fragment respectively. In a very preferred embodiment, R is C₂-alkyl substituted one time with COOH, wherein said COOH group serve as the point for covalent attachment to the X² linking moiety and the PEG fragment respectively.

In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In a further preferred embodiment, said PSMA targeting fragment is a folate ligand. In a further preferred embodiment, said PSMA targeting fragment is a small molecule PSMA targeting fragment, wherein said small molecule PSMA targeting fragment is a folate ligand.

In preferred embodiments, said folate ligand binds to a cell surface receptor, wherein said cell surface receptor is PSMA. As recently reported, targeting of cells expressing PSMA has been achieved by amides of folic acid (Flores 0 et al., Theranostics 2017, 7(9):2477-2494).

As used herein, the term “folate ligand” is understood as folic acid or methotrexate or a derivative or analogue thereof. Preferably said folic acid or methotrexate derivative or analogue thereof comprises a glutamate functionality R—NH—[CH(COOH)—CH₂—CH₂—C(O)NH]_{q}—CH(COOH)—CH₂—CH₂—COOH, wherein η is an integer from 0 to 100, and wherein R is a group of Formula 2:

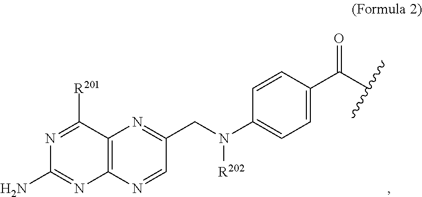

wherein
R²⁰¹ is —OH or —NH₂;
R²⁰² is —H or —CH₃; and
the wavy line indicates the point of attachment to said glutamate functionality. In preferred embodiments, η is an integer from 0 to 10, preferably η is an integer from 0 to 5, and further preferably η is 0.

One of skill in the art will understand that when R²⁰¹ is —OH, in preferred embodiments said OH will tautomerize to a carbonyl group (═O), and the neighboring nitrogen atom of said R²⁰¹ will be protonated.

One of skill in the art will further understand that said glutamate functionality R—NH—[CH(COOH)—CH₂—CH₂—C(O)NH]_{η}—CH(COOH)—CH₂—CH₂—COOH comprises at least one alpha carboxylate group and a gamma carboxylate group. Specifically, the one or more —COOH groups bonded to the same carbon as the —NH—group or groups are understood herein as alpha carboxylate groups. When η=0, the —COOH group bonded to the same carbon as the R—NH group is understood herein as the alpha carboxylate group. The —COOH group bonded to the —(CH₂)₂—group is understood herein as the gamma carboxylate group. Moreover, one of skill in the art will understand that the carboxylate groups discussed herein, e.g., the alpha and the gamma carboxylate groups, can be protonated or deprotonated depending on the pH of the surrounding solution. Accordingly, one of skill in the art will understand that although the carboxylate groups are drawn as neutral species (—COOH) for simplicity and clarity, these can exist (e.g., can primarily exist) as deprotonated, i.e., negatively charged species (—COO—) at physiological pH.

In some embodiments, an alpha carboxylate group of said glutamate functionality serves as the point of covalent attachment to the X² linking moiety. In preferred embodiments, when said alpha carboxylate group of said glutamate functionality serves as said point of attachment to the X² linking moiety, said alpha carboxylate group is condensed with an amine group of the X² linking moiety to form an amide. In some embodiments, when said alpha carboxylate group of said glutamate functionality serves as said point of attachment to the X² linking moiety, said alpha carboxylate group is condensed with a hydroxy group of the X² linking moiety to form an ester.

In preferred embodiments, the gamma carboxylate group of said glutamate functionality serves as the point of covalent attachment to the X² linking moiety. In preferred embodiments, when said gamma carboxylate group of said glutamate functionality serves as said point of attachment to the X² linking moiety, said gamma carboxylate group is condensed with an amine group of the X² linking moiety to form an amide. In some embodiments, when said gamma carboxylate group of said glutamate functionality serves as said point of attachment to the X² linking moiety, said gamma carboxylate group is condensed with a hydroxy group of the X² linking moiety to form an ester.

In a preferred embodiment, said folate ligand is folic acid:

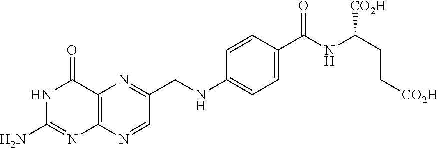 [SMILES: Nc1nc2ncc(CNc3ccc(C(=O)N[C@@H](CCC(=O)O)C(=O)O)cc3)nc2c(=O)[nH]1]

wherein either the alpha carboxylate group or the gamma carboxylate group of said folic acid serves as the point of covalent attachment to the X² linking moiety.

In some embodiments, the alpha carboxylate group of said folic acid serves as the point of covalent attachment to the X² linking moiety. In preferred embodiments, when said alpha carboxylate group of said folic acid serves as said point of attachment to the X² linking moiety, said alpha carboxylate group is condensed with an amine group of the X² linking moiety to form an amide. In some embodiments, when said alpha carboxylate group of said folic acid serves as said point of attachment to the X² linking moiety, said alpha carboxylate group is condensed with a hydroxy group of the X² linking moiety to form an ester.

In preferred embodiments, the gamma carboxylate group of said folic acid serves as the point of covalent attachment to the X² linking moiety. In preferred embodiments, when said gamma carboxylate group of said folic acid serves as said point of attachment to the X² linking moiety, said gamma carboxylate group is condensed with an amine group of the X² linking moiety to form an amide. In some embodiments, when said gamma carboxylate group of said folic acid serves as said point of attachment to the X² linking moiety, said gamma carboxylate group is condensed with a hydroxy group of the X² linking moiety to form an ester.

In a preferred embodiment, said folate ligand is methotrexate:

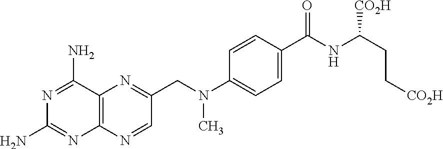 [SMILES: CN(Cc1cnc2nc(N)nc(N)c2n1)c1ccc(C(=O)N[C@@H](CCC(=O)O)C(=O)O)cc1]

wherein either the alpha carboxylate group or the gamma carboxylate group of said methotrexate serves as the point of covalent attachment to the X² linking moiety.

In some embodiments, the alpha carboxylate group of said methotrexate serves as the point of covalent attachment to the X² linking moiety. In preferred embodiments, when said alpha carboxylate group of said methotrexate serves as said point of attachment to the X² linking moiety, said alpha carboxylate group is condensed with an amine group of the X² linking moiety to form an amide. In some embodiments, when said alpha carboxylate group of said methotrexate serves as said point of attachment to the X² linking moiety, said alpha carboxylate group is condensed with a hydroxy group of the X² linking moiety to form an ester.

In preferred embodiments, the gamma carboxylate group of said methotrexate serves as the point of covalent attachment to the X² linking moiety. In preferred embodiments, when said gamma carboxylate group of said methotrexate serves as said point of attachment to the X² linking moiety, said gamma carboxylate group is condensed with an amine group of the X² linking moiety to form an amide. In some embodiments, when said gamma carboxylate group of said methotrexate serves as said point of attachment to the X² linking moiety, said gamma carboxylate group is condensed with a hydroxy group of the X² linking moiety to form an ester. In a further aspect, the present invention provides a composition comprising a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate: R¹—(NR²—CH₂—CH₂)ₙ—Z—X¹—(O—CH₂—CH₂)ₘ—X²-L (Formula I*); wherein n is any integer between 1 and 1500, preferably any integer between 2 and 1500; m is any integer between 1 and 200, preferably m is any integer between 1 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ-moieties is H; X¹ and X² are independently divalent covalent linking moieties; Z is a divalent covalent linking moiety wherein Z is not —NHC(O)—, wherein preferably Z is a divalent covalent linking moiety wherein Z is not a single bond and Z is not —NHC(O)—; L is a targeting fragment capable of binding to a cell overexpressing prostate specific membrane antigen (PSMA), wherein preferably said L is the DUPA residue (HOOC(CH₂)₂-CH(COOH)—NH—CO—NH—CH(COOH)—(CH₂)₂-CO—), and wherein said nucleic acid is a pharmaceutically active nucleic acid, wherein said pharmaceutically active nucleic acid is a nucleic acid that encodes a pharmaceutically active peptide or protein, and wherein preferably said composition consists of said conjugate.

In a further aspect, the present invention provides a composition comprising a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate: R¹—(NR²—CH₂—CH₂)ₙ—Z—X¹—(O—CH₂—CH₂)ₘ—X²-L (Formula I*); wherein n is any integer between 1 and 1500, preferably any integer between 2 and 1500; m is any integer between 1 and 200, preferably m is any integer between 1 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ-moieties is H; X¹ and X² are independently divalent covalent linking moieties; Z is a divalent covalent linking moiety wherein Z is not —NHC(O)—, wherein preferably Z is a divalent covalent linking moiety wherein Z is not a single bond and Z is not —NHC(O)—; L is a targeting fragment capable of binding to prostate specific membrane antigen (PSMA), wherein preferably said L is the DUPA residue (HOOC(CH₂)₂—CH(COOH)—NH—CO—NH—CH(COOH)—(CH₂)₂—CO—), and wherein said nucleic acid is a pharmaceutically active nucleic acid, wherein said pharmaceutically active nucleic acid is a nucleic acid that encodes a pharmaceutically active peptide or protein, and wherein preferably said composition consists of said conjugate.

In a further aspect, the present invention provides a composition comprising a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate: R¹—(NR²—CH₂—CH₂)ₙ—Z—X¹—(O—CH₂—CH₂)ₘ—X²-L (Formula I*); wherein n is any integer between 1 and 1500, preferably any integer between 2 and 1500; m is any integer between 1 and 200, preferably m is any integer between 1 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ-moieties is H; X¹ and X² are independently divalent covalent linking moieties; Z is a divalent covalent linking moiety wherein Z is not —NHC(O)—, wherein preferably Z is a divalent covalent linking moiety wherein Z is not a single bond and Z is not —NHC(O)—; L is a targeting fragment, wherein said targeting fragment L is the DUPA residue (HOOC(CH₂)₂-CH(COOH)—NH—CO—NH—CH(COOH)—(CH₂)₂-CO—), and wherein said nucleic acid is a pharmaceutically active nucleic acid, wherein said pharmaceutically active nucleic acid is a nucleic acid that encodes a pharmaceutically active peptide or protein, and wherein preferably said composition consists of said conjugate.

In another aspect, the present invention provides a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

R¹—(NR²—CH₂—CH₂)ₙ—Z—X¹—(O—CH₂—CH₂)ₘ—X²-L (Formula I*); wherein n is any integer between 1 and 1500 preferably any integer between 2 and 1500; m is any integer between 1 and 200, preferably m is any integer between 1 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ-moieties is H; X¹ and X² are independently divalent covalent linking moieties; Z is a divalent covalent linking moiety wherein Z is not —NHC(O)—, wherein preferably Z is a divalent covalent linking moiety wherein Z is not a single bond and Z is not —NHC(O)—; L is a targeting fragment capable of binding to a cell overexpressing prostate specific membrane antigen (PSMA), wherein preferably said L is the DUPA residue (HOOC(CH₂)₂-CH(COOH)—NH—CO—NH—CH(COOH)—(CH₂)₂-CO—), and wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

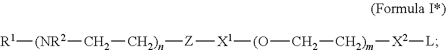

wherein n is any integer between 1 and 1500, preferably any integer between 2 and 1500; m is any integer between 1 and 200, preferably m is any integer between 1 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ-moieties is H; X¹ and X² are independently divalent covalent linking moieties; Z is a divalent covalent linking moiety wherein Z is not —NHC(O)—, wherein preferably Z is a divalent covalent linking moiety wherein Z is not a single bond and Z is not —NHC(O)—; L is a targeting fragment capable of binding to prostate specific membrane antigen (PSMA), wherein preferably L is the DUPA residue (HOOC(CH₂)₂-CH(COOH)—NH—CO—NH—CH(COOH)—(CH₂)₂-CO—), and wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I* or a pharmaceutically acceptable salt, solvate, hydrate, tautomer or enantiomer thereof, and a nucleic acid, wherein said nucleic acid is preferably non-covalently bound to said conjugate:

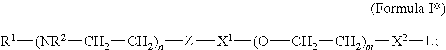

wherein n is any integer between 1 and 1500, preferably any integer between 2 and 1500; m is any integer between 1 and 200, preferably m is any integer between 1 and 100; R¹ is an initiation residue, wherein preferably R¹ is —H or —CH₃; R² is independently —H or an organic residue, wherein at least 80%, preferably 90% of said R² in said —(NR²—CH₂—CH₂)ₙ-moieties is H; X¹ and X² are independently divalent covalent linking moieties; Z is a divalent covalent linking moiety wherein Z is not —NHC(O)—, wherein preferably Z is a divalent covalent linking moiety wherein Z is not a single bond and Z is not —NHC(O)—; L is a targeting fragment, wherein said targeting fragment L is the DUPA residue (HOOC(CH₂)₂—CH(COOH)—NH—CO—NH—CH(COOH)—(CH₂)₂—CO—), and wherein said nucleic acid is a pharmaceutically active nucleic acid, wherein said pharmaceutically active nucleic acid is a nucleic acid that encodes a pharmaceutically active peptide or protein.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

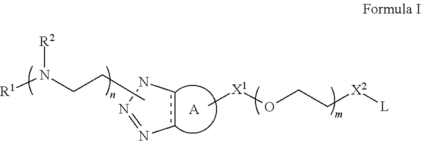

wherein:
[symbol] is a single bond or a double bond;
n is any integer between 1 and 1500;
m is any integer between 1 and 200, preferably any integer between 2 and 1500;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ-moieties is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two
R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2};
R^{A2} is independently selected from C₁-C₆ alkyl, C₁-C₆ alkoxy, halogen —SO₃H, or —OSO₃H;
X¹ is a linking moiety of the formula —(Y¹)ₚ—, wherein p is an integer between 1 and 20, and each occurrence of Y¹ is independently selected from a chemical bond, —CR¹¹R¹²—, —C(O)—, —O—, —S—, —NR¹³—, an amino acid residue, a divalent phenyl moiety, a divalent carbocycle moiety, a divalent heterocycle moiety, and a divalent heteroaryl moiety, wherein each divalent phenyl or heteroaryl is optionally substituted with one or more R¹³, and each divalent heterocycle is optionally substituted with one or more R¹⁴; wherein R¹¹, R¹² and R¹³ are independently, at each occurrence, H, —SO₃H, —NH₂, —CO₂H, or C₁-C₆ alkyl, wherein each alkyl is optionally substituted with —CO₂H or —NH₂; and wherein R¹⁴ is independently, at each occurrence, H, C₁-C₆ alkyl, or oxo, C₆—Cₘ₀ aryl, or 5 to 8-membered heteroaryl;
X² is a linking moiety of the formula —(Y²)_{q}—, wherein q is an integer between 1 and 50, and each occurrence of Y² is independently selected from a chemical bond, —CR²¹R²²—, NR²³—, —O—, —S—, —C(O)—, an amino acid residue, a divalent phenyl moiety, a divalent carbocycle moiety a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴; wherein R²¹, R²², and R²³ are each independently, at each occurrence, —H, —SO₃H, —NH₂, —CO₂H, or C₁-C₆ alkyl, wherein each C₁-C₆ alkyl is optionally substituted with one or more —OH, oxo, —CO₂H, —NH₂, C₆-C₁₀ aryl, or 5 to 8-membered heteroaryl; and wherein R²⁴ is independently, at each occurrence, —H, —CO₂H, C₁-C₆ alkyl, or oxo; and
L is a targeting fragment, wherein preferably said targeting fragment L is the DUPA residue (HOOC(CH₂)₂-CH(COOH)—NH—CO—NH—CH(COOH)—(CH₂)₂-CO—), and wherein said nucleic acid is a pharmaceutically active nucleic acid, wherein said pharmaceutically active nucleic acid is a nucleic acid that encodes a pharmaceutically active peptide or protein.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

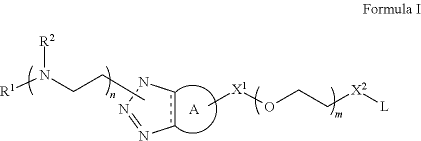

wherein:
[symbol] is a single bond or a double bond;
n is any integer between 1 and 1500, preferably any integer between 2 and 1500;
m is a discrete number of repeating units m of 2 to 100, preferably of a discrete number of repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

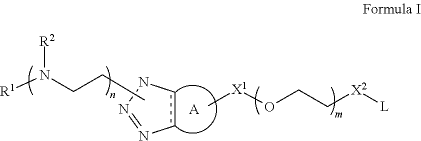

wherein:
[symbol] is a single bond or a double bond;
n is any integer between 1 and 1500, preferably any integer between 2 and 1500;
m is a discrete number of repeating units m of 2 to 100, preferably of a discrete number of repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂-CH(COOH)—NH—CO—NH—CH(COOH)—(CH₂)₂-CO—). In a further very preferred embodiment, said targeting fragment consists of the DUPA residue (HOOC(CH₂)₂-CH(COOH)—NH—CO—NH—CH(COOH)—(CH₂)₂-CO—), wherein both chiral C-atoms having (S)-configuration, as depicted in formula 1*.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

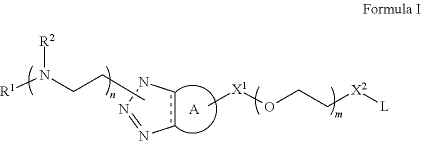

wherein:
[symbol] is a single bond or a double bond;
n is any integer between 1 and 1500, preferably any integer between 2 and 1500;
m is a discrete number of repeating units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein.. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

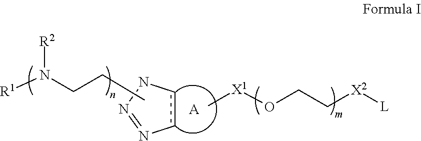

wherein:
[symbol] is a single bond or a double bond;
n is any integer between 1 and 1500, preferably any integer between 2 and 1500;
m is a discrete number of repeating units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

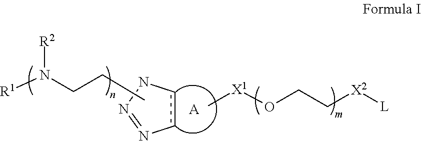

wherein:
[symbol] is a single bond or a double bond;
n is any integer between 1 and 1500, preferably any integer between 2 and 1500;
m is a discrete number of contiguous repeating units m of 2 to 100, preferably of a discrete number of contiguous repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

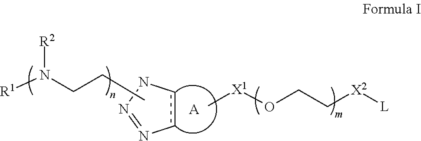

wherein:
[symbol] is a single bond or a double bond;
n is any integer between 1 and 1500, preferably any integer between 2 and 1500;
m is a discrete number of contiguous repeating units m of 2 to 100, preferably of a discrete number of contiguous repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another aspect, the present invention provides a composition comprising a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

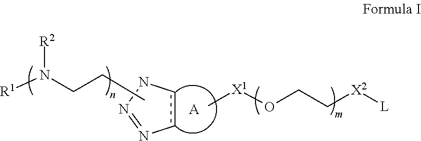

wherein:
[symbol] is a single bond or a double bond;
n is any integer between 1 and 1500, preferably any integer between 2 and 1500;
m is a discrete number of contiguous repeating units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another aspect, the present invention provides a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

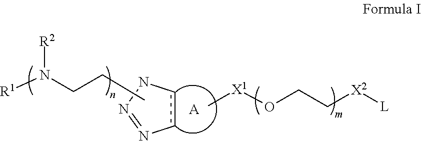

wherein.
[symbol] is a single bond or a double bond;
n is any integer between 1 and 1500, preferably any integer between 2 and 1500;
m is a discrete number of contiguous repeating units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another aspect, the present invention provides a composition comprising, preferably consisting of, a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

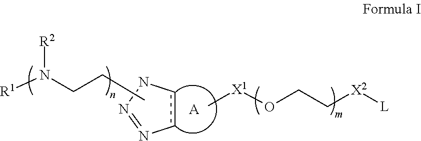

wherein:
[symbol] is a single bond or a double bond;
n is any integer between 1 and 1500, preferably any integer between 2 and 1500;
m is a discrete number of contiguous repeating units m of 2 to 100, preferably of a discrete number of contiguous repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a linking moiety of the formula —(Y¹)ₚ—, wherein p is an integer between 1 and 20, and each occurrence of Y¹ is independently selected from a chemical bond, —CR¹¹R¹²—, —C(O)—, —O—, —S—, —NR¹³—, an amino acid residue, a divalent phenyl moiety, a divalent heterocycle moiety, and a divalent heteroaryl moiety, wherein each divalent phenyl or heteroaryl is optionally substituted with one or more R¹³, and each divalent heterocycle is optionally substituted with one or more R¹⁴; wherein R¹¹, R¹² and R¹³ are independently, at each occurrence, H or C₁-C₆ alkyl; and wherein R¹⁴ is independently, at each occurrence, H, C₁-C₆ alkyl, or oxo;
X² is a linking moiety of the formula —(Y²)_{q}—, wherein q is an integer between 1 and 50, and each occurrence of Y² is independently selected from a chemical bond, —CR²¹R²²—, NR²³—, —O—, —S—, —C(O)—, an amino acid residue, a divalent phenyl moiety, a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴; wherein R²¹, R²², and R²³ are each independently, at each occurrence, —H, —CO₂H, or C₁-C₆ alkyl, wherein each C₁-C₆ alkyl is optionally substituted with one or more —OH, oxo, C₆-C₁₀ aryl, or 5 to 8-membered heteroaryl; and wherein R²⁴ is independently, at each occurrence, —H, —CO₂H, C₁-C₆ alkyl, or oxo; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another aspect, the present invention provides a composition comprising, preferably consisting of, a polyplex, wherein said polyplex comprise a conjugate of the Formula I, or a pharmaceutically acceptable salt, solvate, hydrate, tautomer or enantiomer thereof, and a nucleic acid, wherein said nucleic acid is preferably non-covalently bound to said conjugate:

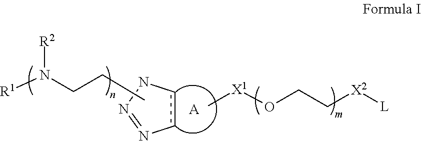

wherein:
[symbol] is a single bond or a double bond;
n is any integer between 1 and 1500, preferably any integer between 2 and 1500;
m is a discrete number of contiguous repeating units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ—is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein said targeting fragment is an PSMA targeting fragment, wherein preferably said PSMA targeting fragment is capable of specifically binding to a cell expressing, preferably overexpressing, PSMA, and wherein said nucleic acid is a pharmaceutically active nucleic acid, wherein said pharmaceutically active nucleic acid is a nucleic acid that encodes a pharmaceutically active peptide or protein. In a preferred embodiment, said R¹ is —H. In a preferred embodiment, said R¹ is —CH₃. In a further preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In a preferred embodiment, said DUPA residue is linked to said PEG targeting fragment by way of the linking moiety X².

Such linking moieties are known to the skilled person and are disclosed in US2020/0188523A1, US2011/0288152A1, US2010/324008A1, the disclosures of said patent applications incorporated herein by way reference in its entirety.

In a preferred embodiment, said linking moiety X² is a peptide linker or a C₁-C₁₀ alkylene linker or a combination of both. In a preferred embodiment, said linking moiety X² is a peptide linker.

In a preferred embodiment, said linking moiety X² is a peptide linker, wherein said peptide linker comprises, preferably consists of, the sequence of SEQ ID NO:3 (—(NH—(CH₂)₇—CO)-Phe-Phe-(NH—CH₂—CH(NH₂)—CO)-Asp-Cys-) or SEQ ID NO:1 (—(NH—(CH₂)₇—CO)-Phe-Gly-Trp-Trp-Gly-Cys-). In a preferred embodiment, said linking moiety X² is a peptide linker, wherein said peptide linker comprises, preferably consists of, the sequence of SEQ ID NO:1 (—(NH—(CH₂)₇—CO)-Phe-Gly-Trp-Trp-Gly-Cys-). In a further preferred embodiment, said linking moiety X² comprises, preferably consists of, SEQ ID NO:1 or SEQ ID NO:3 and the targeting fragment is HOOC(CH₂)₂—CH(COOH)—NH—CO—NH—CH(COOH)—(CH₂)₂—CO— (DUPA residue). In a very preferred embodiment, said linking moiety X² comprises, preferably consists of, SEQ ID NO:1 and the targeting fragment L is HOOC(CH₂)₂—CH(COOH)—NH—CO—NH—CH(COOH)—(CH₂)₂—CO— (DUPA residue). In a preferred embodiment, said targeting fragment L is HOOC—(CH₂)₂—CH(COOH)—NH—CO—NH—CH(COOH)—(CH₂)₂—CO—capable of binding to a cell overexpressing PSMA, wherein said linking moiety X² comprises, preferably consists of SEQ ID NO:1.

In another preferred embodiment, the targeting fragment is 2-[3-(1,3-dicarboxypropyl) ureido]pentanedioic acid (DUPA), wherein typically and preferably said coupling to the rest of said conjugate is effected via a terminal carboxyl group of said DUPA. Thus, in a further preferred embodiment, said targeting fragment L is the DUPA residue (HOOC(CH₂)₂—CH(COOH)—NH—CO—NH—CH(COOH)—(CH₂)₂—CO—). The DUPA can be selectively taken up in cells that have increased expression (e.g., overexpression) of prostate-specific membrane antigen (PSMA).

In a preferred embodiment, said targeting fragment is capable of binding to an asialoglycoprotein receptor (ASGPr), which is also named herein as ASGPr targeting fragment. Thus, in some embodiments said targeting fragment is an ASGPr targeting fragment. Asialoglycoprotein receptors (ASGPr) are carbohydrate binding proteins (i.e., lectins) which bind asialoglycoprotein and glycoproteins, preferably galactose-terminal glycoproteins and preferably branched galactose-terminal glycoproteins. Preferably said ASGPr targeting fragment is capable of binding to epitopes on the extracellular domain of ASGPr.

Preferably, said ASGPr targeting fragment is capable of binding to a cell expressing ASGPr. In a preferred embodiment, said targeting fragment is capable of binding to a cell overexpressing ASGPr, preferably a hepatocyte. In a preferred embodiment, said targeting fragment is capable of binding to a cell ASGPr expressing. In a preferred embodiment, said targeting fragment is capable of binding to a cell overexpressing ASGPr. In one embodiment, said cell overexpressing ASGPr means that the level of ASGPr expressed in said cell of a certain tissue is elevated in comparison to the level of ASGPr as measured in a normal healthy cell of the same type of tissue under analogous conditions. In one embodiment, said cell overexpressing ASGPr refers to an increase in the level of ASGPr in a cell relative to the level in the same cell or closely related non-malignant cell under normal physiological conditions. In one embodiment, said cell overexpressing ASGPr relates to expression of ASGPr that is at least 5-fold, preferably at least 10-fold, further preferably at least 20-fold, as compared to the expression of ASGPr in a normal cell or in a normal tissue. For example, ASGPr is overexpressed in liver cells, preferably hepatocytes, and liver cancer cells. In preferred embodiments, the ASGPr targeting fragment is capable of binding to a liver cell, preferably a hepatocyte or cancerous liver cell and metastases thereof.

Preferably said ASGPr targeting fragment is capable of specifically binding to ASGPr. Typically, specific binding refers to a binding affinity or dissociation constant (KD) of the targeting fragment between about 1×10⁻³ M and about 1×10⁻¹² M. To detect binding of the complex or measure affinity, molecules can be analyzed using a competition binding assay, such as with a Biacore 3000 instrument (see, e.g., Kuo et al., PLoS One, 2015; 10(2): e01166610). Preferably said ASGPr targeting fragment is capable of specifically binding to ASGPr with a binding affinity equal to or greater than that of galactose.

In a preferred embodiment, said ASGPr targeting fragments include small molecules or small molecule ligand, peptides, proteins, more preferably ASGPr antibodies, ASGPr affibodies, ASGPr aptamers, ASGPr targeting peptides, lactose, galactose, N-acetylgalactosamine (GalNAc), galactosamine, N-formylgalactosamine, N-acetyl-galactosamine, N-propionylgalactosamine, N-n-butanoylgalactosamine, and N-iso-butanoylgalactosamine, and combinations thereof (Iobst, S. T. and Drickamer, K. J. B. C. 1996, 271, 6686). In some embodiments, ASGPr targeting fragments are monomeric (i.e., having a single galactosamine). In some embodiments, ASGPr targeting fragments are multimeric (i.e., having multiple galactosamines).

In a preferred embodiment, the ASGPr targeting fragment is a galactose cluster. A galactose cluster is understood as a molecule having two to four terminal galactose derivatives. As used herein, the term galactose derivative includes both galactose and derivatives of galactose having affinity for the asialoglycoprotein receptor equal to or greater than that of galactose. Preferably the galactose derivative is selected from galactose, galactosamine, N-formylgalactosamine, N-acetylgalactosamine, N-propionyl-galactosamine, N-n-butanoylgalactosamine, and N-iso-butanoylgalactosamine. Preferably the galactose derivative is an N-acetyl-galactosamine (GalNAc).

In preferred embodiments, a galactose cluster contains three galactose derivatives each linked to a central branch point, preferably wherein each terminal galactose derivative is attached to the remainder of the galactose cluster through its C-1 carbon. In preferred embodiments, the galactose derivative is linked to the branch point via linkers or spacers, preferably flexible hydrophilic spacers, more preferably PEG spacers and yet more preferably PEG3 spacers.

In preferred embodiments, a galactose cluster has three terminal galactosamines or galactosamine derivatives each having affinity for the ASGPr (i.e., is a tri-antennary galactose derivative cluster). In some embodiments the galactose cluster comprises tri-antennary galactose, tri-valent galactose and galactose trimer. Preferably the galactose cluster has three terminal N-acetyl-galactosamines.

In another preferred embodiment, the targeting fragment is folic acid, wherein typically and preferably said coupling to the rest of said conjugate is effected via the terminal carboxyl group of said folic acid. In some preferred embodiments, the targeting fragment can be folate. Without wishing to be bound by theory, folate can be selectively taken up in cells that have increased expression (e.g., overexpression) of folate receptor.

In further preferred embodiments the targeting fragment are HER2 targeting ligands, which in some embodiments can be selectively taken up in cells that have increased expression (e.g., overexpression) of HER2.

In some embodiments, the targeting fragment can be a somatostatin receptor-targeting fragment. Without wishing to be bound by theory, the somatostatin receptor-targeting fragment can be selectively taken up by cells that have increased expression (e.g., overexpression) of somatostatin receptors such as somatostatin receptor 2 (SSTR2).

In some embodiments, the targeting fragment can be an integrin-targeting fragment such as arginine-glycine-aspartic acid (RGD)-containing ligands (e.g., cyclic RGD ligands). Without wishing to be bound by theory, the integrin-targeting fragment can be selectively taken up by cells that have increased expression (e.g., overexpression) of integrins (e.g., RGD integrins such as αᵥβ₆ integrin or αᵥβ₈ integrin).

In some embodiments, the targeting fragment can be a low pH insertion peptides (pHLIP). Without wishing to be bound by theory, the low pH insertion peptide can be selectively taken up by cells that exist in a low pH microenvironment. In some embodiments, the targeting fragment can be an asialoglycoprotein receptor-targeting fragment such as asialoorosomucoid. Without wishing to be bound by theory, the asialoglycoprotein receptor-targeting fragment can be selectively taken up by cells that have increased expression (e.g., overexpression) of asialoglycoprotein receptors. In some embodiments, the targeting fragment can be an insulin-receptor targeting fragment such as insulin. Without wishing to be bound by theory, the insulin-receptor targeting fragment can be selectively taken up by cells that have increased expression (e.g., overexpression) of insulin receptors. In some embodiments, targeting fragment can be a mannose-6-phosphate receptor targeting fragment such as mannose-6-phosphate. Without wishing to be bound by theory, the mannose-6-phosphate receptor targeting fragment can be selectively taken up by cells that have increased expression (e.g., overexpression) of mannose-6-phosphate receptors (e.g., monocytes). In some embodiments, the targeting fragment can be a mannose receptor-targeting fragment such as mannose. Without wishing to be bound by theory, the mannose-receptor-targeting fragment can be selectively taken up by cells that have increased expression (e.g., overexpression) of mannose receptors. In some embodiments, the targeting fragment can be a Sialyl Lewis^{x} antigen targeting fragments such as E-selectin. Without wishing to be bound by theory, the Sialyl Lewis^{x} antigen-targeting fragments can be selectively taken up by cells that have increased expression (e.g., overexpression) of glycosides such as Sialyl Lewis^{x} antigens. In some embodiments, the targeting fragment can be N-acetyllactosamine targeting fragment. Without wishing to be bound by theory, the N-acetyllactosamine targeting fragment can be selectively taken up by cells that have increased expression (e.g., overexpression) N-acetyllactosamine. In some embodiments, the targeting fragment can be a galactose targeting fragment. Without wishing to be bound by theory, the galactose targeting fragment can be selectively taken up by cells that have increased expression (e.g., overexpression) of galactose. In some embodiments, the targeting fragment can be a sigma-2 receptor agonist, such as N,N-dimethyltryptamine (DMT), a sphingolipid-derived amine, and/or a steroid (e.g., progesterone). Without wishing to be bound by theory, the sigma-2 receptor agonist can be selectively taken up by cells that have increased expression (e.g., overexpression) of sigma-2 receptors. In some embodiments, the targeting fragment can be a p32-targeting ligand such as anti-p32 antibody or p32-binding LyP-1 tumor-homing peptide. Without wishing to be bound by theory, the p32-targeting ligand can be selectively taken up by cells that have increased expression (e.g., overexpression) of the mitochondrial protein p32. In some embodiments, the targeting fragment can be a Trop-2 targeting fragment such as an anti-Trop-2 antibody and/or antibody fragment. Without wishing to be bound by theory, the Trop-2 targeting fragment can be selectively taken up by cells that have increased expression (e.g., overexpression) of Trop-2. In some embodiments, the targeting fragment is an insulin-like growth factor 1 receptor-targeting fragment, such as insulin-like growth factor 1. Without wishing to be bound by theory, the insulin-like growth factor 1 receptor-targeting fragment can be selectively taken up by cells that have increased expression (e.g., overexpression) of insulin-like growth factor 1 receptor. In some embodiments, the targeting fragment can be a VEGF receptor-targeting fragment such as VEGF. Without wishing to be bound by theory, the VEGF receptor-targeting fragment can be selectively taken up by cells that have increased expression (e.g., overexpression) of VEGF receptor. In some embodiments, the targeting fragment can be a platelet-derived growth factor receptor-targeting fragment such as platelet-derived growth factor. Without wishing to be bound by theory, the platelet-derived growth factor receptor-targeting fragment can be selectively taken up by cells that have increased expression (e.g., overexpression) of platelet-derived growth factor receptor. In some embodiments, the targeting fragment can be a fibroblast growth factor receptor-targeting fragment such as fibroblast growth factor. Without wishing to be bound by theory, the fibroblast growth factor receptor-targeting fragment can be selectively taken up by cells that have increased expression (e.g., overexpression) of fibroblast growth factor receptor.

### Coupling of PEG Fragment to Targeting Fragment

In some embodiments, the second terminal end of the PEG fragment is functionalized with a linking group (i.e., X²) that links the PEG fragment to a targeting fragment. Typically, the linking moiety X² comprises a reactive group for coupling to an appropriate, i.e. complementary reactive group on the targeting fragment. One of skill in the art will understand the various complementary reactive groups of such coupling reaction between said X² reactive groups and said reactive groups of the targeting fragments. In some embodiments, the targeting fragment L can be unmodified and used directly as a reactive partner for covalent coupling to a PEG fragment and linking moiety X² respectively. For example, Scheme 3 shows the nucleophilic addition of hEGF to an electrophilic tetrafluorophenyl ester bonded to a PEG fragment. As shown in Scheme 3, a nucleophilic amine of the hEGF displaces the tetrafluorophenol of the tetrafluorophenyl ester to form a covalent bond with the PEG fragment and linking moiety X² respectively. In some embodiments, the targeting fragment L can be coupled to a PEG fragment by the linking moiety X² using a suitable chemical linkage such as an amide or ester bond. For example, Schemes 4 and 5 show DUPA and folate groups, respectively, that are bonded to a PEG fragment by an X² linker comprising an amide linkage. The amide groups are formed by a dehydration synthesis reaction between an appropriate carboxylic acid group on DUPA and folate and an appropriate amine on the PEG-X² fragment.

In some preferred embodiments, a first end (i.e., terminus) of the PEG fragment is functionalized with an alkene or alkyne group which can in some embodiments be used to react with an azide-functionalized LPEI; and a second end (i.e., terminus) of the PEG fragment is functionalized with a targeting fragment, which in some embodiments can be used to facilitate uptake of the conjugates and corresponding polyplexes in specific cell types. Accordingly, in some preferred embodiments, the resulting conjugates of the present invention can have the general structure LPEI-PEG-Targeting fragment, arranged in a linear end-to-end fashion.

The conjugates of the present invention can be prepared using a variety of different methods and steps. Schemes 1 and 2 below show different strategies for arranging the conjugates of the present invention. As shown below in Scheme 1, conjugates of the present invention can be prepared by first coupling a PEG fragment to a targeting fragment, followed by coupling targeting fragment-modified PEG fragment to the LPEI fragment. As shown below in Scheme 2, conjugates of the present invention can be prepared by first coupling a PEG fragment to the LPEI fragment, followed by coupling the LPEI-modified PEG fragment to a targeting fragment.

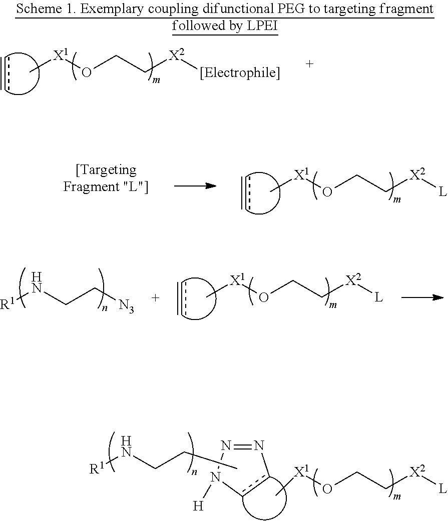

As shown in Scheme 1, a difunctional PEG (e.g, a PEG containing an alkene or alkyne and an electrophile) can be reacted first with a targeting fragment (e.g., hEGF, DUPA, or folate) to produce a PEG fragment covalently bonded to the targeting fragment. The alkene or alkyne group of the targeting fragment-modified PEG can then be reacted with the azide group of an LPEI fragment via a [3+2]cycloaddition to produce a linear conjugate of the general structure LPEI-PEG-targeting fragment.

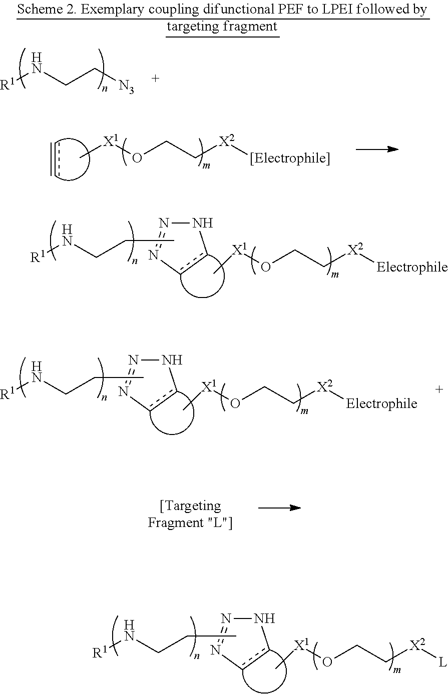

As shown in Scheme 2, a bifunctional PEG (e.g., a PEG containing an alkene or alkyne and an electrophile) can be reacted first with the azide group of an LPEI fragment via a [3+2]cycloaddition to produce a linear conjugate of LPEI and PEG covalently attached by a 1, 2, 3 triazole or A 4,5-dihydro-1H-[1,2,3]triazole. The linear LPEI-PEG fragment can then be reacted with a targeting fragment (e.g., hEGF, DUPA, or folate) to produce a linear conjugate of the general structure LPEI-PEG-targeting fragment.

Schemes 3-5 below show general methods for coupling a PEG fragment to various targeting fragments. One of skill in the art will appreciate that the PEG fragment can be coupled to various targeting fragments using any suitable chemistries (e.g., nucleophilic substitution, peptide coupling and the like). For example, one of skill in the art will appreciate that it is not necessary to use a tetrafluorophenyl ester as an electrophile to couple a PEG fragment to hEGF as shown in Scheme 3, but that other electrophilic groups such as a maleate (as shown in Scheme 4) can also be used. Moreover, one of skill in the art will appreciate that the reactive group of the bi-functionalized PEG fragment does not necessarily need to be an electrophilic group, but instead can be a nucleophilic group that reacts, e.g., with an electrophilic portion of a targeting fragment.

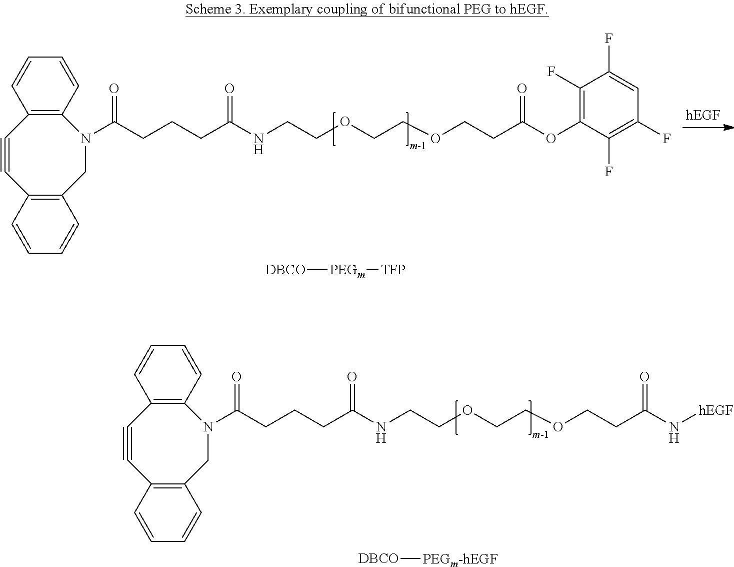

As shown above in Scheme 3, in some embodiments PEG can be modified to include an electrophilic group such as a tetrafluorophenyl ester and/or an activated alkyne group such as DBCO. Treatment of the tetrafluorophenyl ester-modified PEG with hEGF in solution results in a nucleophilic substitution via a nucleophilic amine of hEGF to produce an hEGF-modified PEG. The DBCO group can be used in subsequent reactions for coupling to an LPEI fragment. The variable m represents the number of repeating PEG units as described herein.

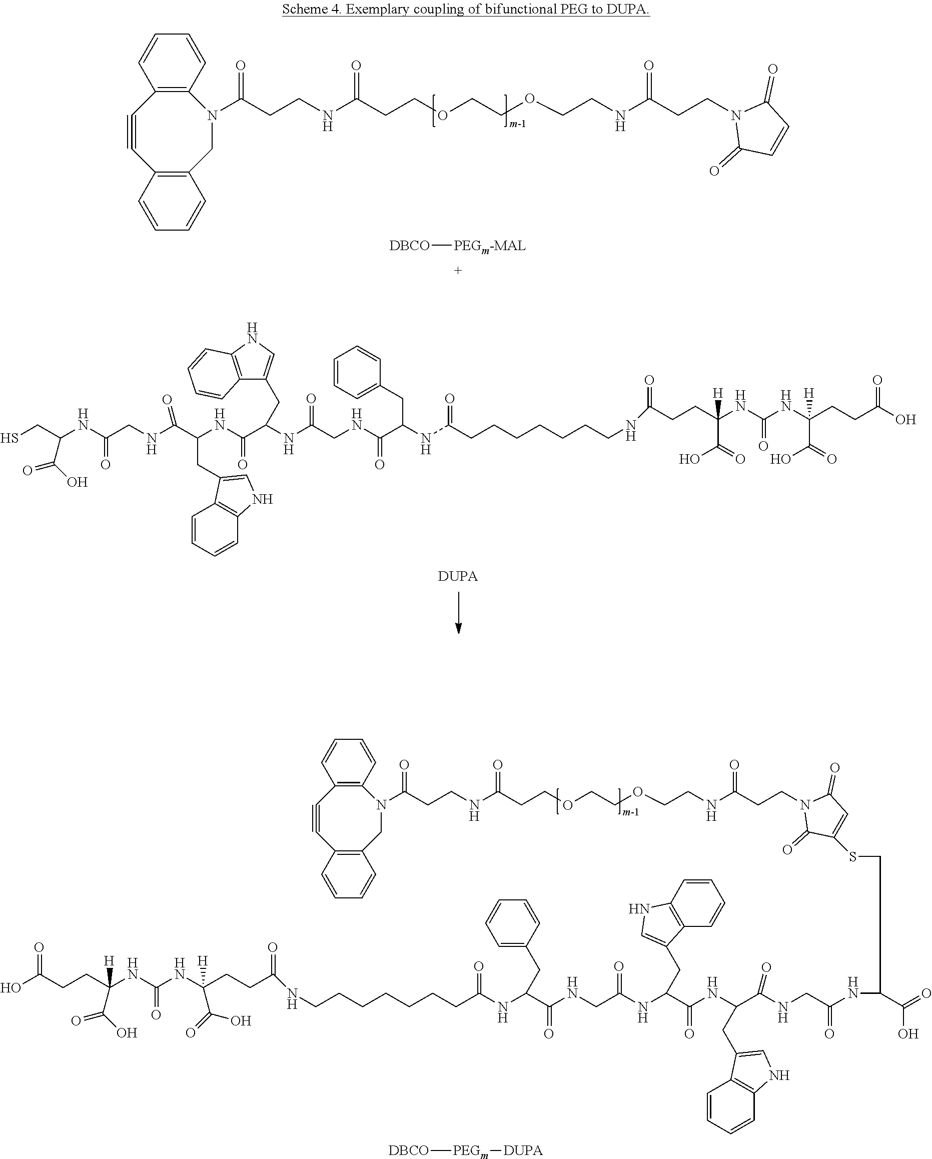

As shown above in Scheme 4, PEG can be modified to include an electrophilic maleimide (MAL) group and/or an activated alkyne group such as DBCO. The maleimide-substituted PEG can be coupled to a nucleophilic partner such as the depicted DUPA derived moiety (as depicted in the scheme above comprising a peptidic spacer Aoc-Phe-Gly-Trp-Trp-Gly-Cys (SEQ ID NO:1), N-terminally derivatized with 2-[3-(1,3-dicarboxypropyl)ureido]pentanedioic acid (DUPA) which due to the amino acid residue derived from cysteine contains a nucleophilic group, namely a thiol. Treatment of the MAL-modified PEG in solution with the thiol-modified DUPA derived moiety in solution results in a nucleophilic 1,4-addition via the nucleophilic thiol of the DUPA derived moiety to produce a DUPA-modified PEG. The variable m represents the number of repeating PEG units as described herein.

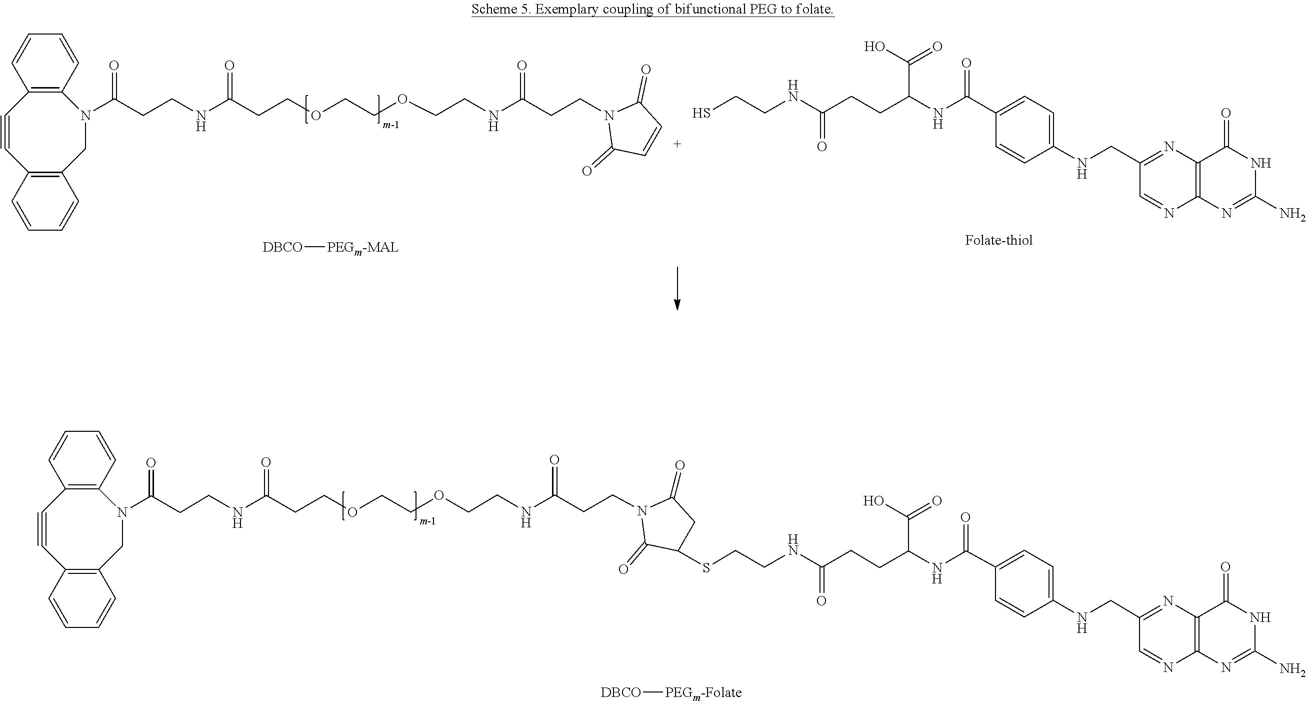

As shown above in Scheme 5, PEG can be modified to include an electrophilic maleimide (MAL) group. The maleimide-substituted PEG can be coupled to nucleophilic partner such as a folate residue which itself is modified to contain a nucleophilic group (e.g., thiol). Treatment of the MAL-modified PEG in solution with folate thiol in solution results in a nucleophilic 1,4-addition via the nucleophilic thiol of folate to produce a folate-modified PEG. The variable m represents the number of repeating PEG units as described herein.

### Coupling of PEG Fragment to LPEI Fragment

Before or after coupling the bi-functionalized PEG fragment to a targeting fragment, the bi-functionalized PEG fragment can be coupled to an LPEI fragment. In preferred embodiments, the bi-functionalized PEG fragment is coupled to LPEI using cycloaddition chemistry, e.g., a 1,3-dipolar cycloaddition or [3+2]cycloaddition between an azide and an alkene or alkyne to form a 1, 2, 3 triazole or a 4,5-dihydro-1H-[1,2,3]triazole. In other preferred embodiments, the bi-functionalized PEG fragment is coupled to LPEI using thiol-ene chemistry, between a thiol and an alkene to form a thioether.

One of skill in the art will appreciate that any suitable alkene or alkyne groups can be used to react with an azide group to couple the LPEI fragment to the PEG fragment. In some preferred embodiments, incorporation of alkene or alkyne groups into ring systems introduces strain into the ring systems. The strain of the ring systems can be released upon reaction of the alkene or alkyne group to produce a 1, 2, 3 triazole or a 4,5-dihydro-1H-[1,2,3]triazole, preferably without the use of an added catalyst such as copper. Thus, in some preferred embodiments, suitable ring systems include seven-, eight-, or nine-membered rings that include an alkyne group, or eight-membered rings that include a trans alkene group. For example, suitable alkyne groups such as cyclooctyne (OCT), monofluorinated cyclooctyne (MOFO), difluorocycloalkyne (DIFO), dibenzocyclooctynol (DIBO), dibenzoazacyclooctyne (DIBAC), bicyclononyne (BCN), biarylazacyclooctynone (BARAC) and tetramethylthiepinium (TMTI) can be used. Additionally, suitable alkene groups such as trans cyclooctene, trans cycloheptene, and maleimide can be used. For example, conjugates of the present invention can be prepared from moieties comprising a PEG fragment and an alkene or alkyne group according to one of the following formulae:

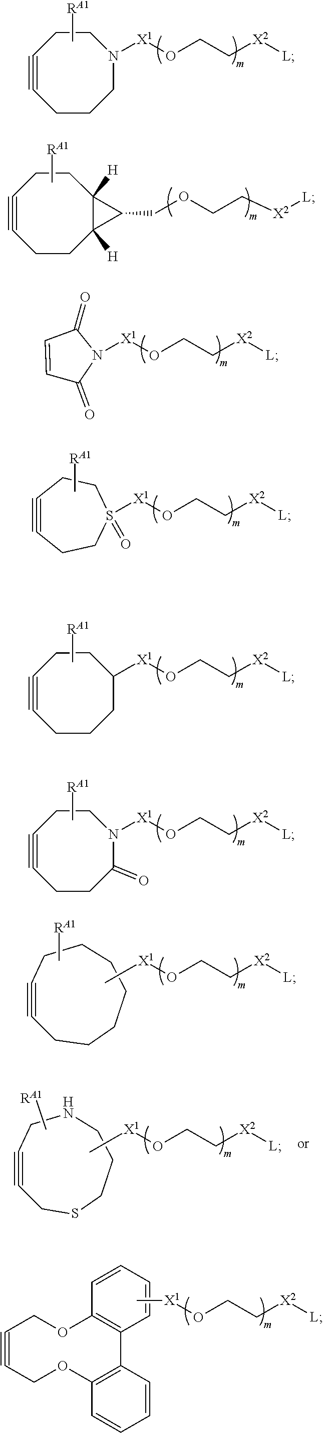

wherein the variables X¹, X², R^{A1}, L and m are defined above.

Without wishing to be bound by theory, the azide and the alkene or alkyne groups can spontaneously (i.e., without the addition of a catalyst) react to form a 1, 2, 3 triazole or a 4,5-dihydro-1H-[1,2,3]triazole. In some embodiments, the azide group reacts with an alkyne to form a 1, 2, 3 triazole. In some embodiments, the azide group reacts with an alkene to form a 4,5-dihydro-1H-[1,2,3]triazole.

One of skill in the art will appreciate that both the LPEI fragment and the PEG fragment can be functionalized to include an azide group, and both the LPEI fragment and the PEG fragment can be functionalized to include an alkene or alkyne fragment (e.g., a strained alkene or alkyne). Thus, in some embodiments, the LPEI fragment comprises the alkene or alkyne group (e.g., a strained alkene or alkyne) and the bi-functionalized PEG fragment comprises an azide group. In some preferred embodiments, the bi-functionalized PEG fragment comprises the alkene or alkyne group (e.g., a strained alkene or alkyne) and the LPEI fragment comprises an azide group.

One of skill in the art will also appreciate that a [3+2]cycloaddition between an azide and an alkene or alkyne group can give adducts with different regiochemistries as shown in Schemes 6-8, below. One of skill in the art will understand that all possible regiochemistries of [3+2]cycloaddition are contemplated by this invention.

In some preferred embodiments, the [3+2]azide-alkyne cycloaddition reaction takes place at a pH of 5 or below, preferably 4 or below. As set forth below in the Comparative Example, no reaction occurred when a PEG fragment modified with an activated alkyne was treated with a non-azide containing LPEI fragment at a pH of 4. Without wishing to be bound by theory, these results suggest that the azide group of the LPEI fragment chemoselectively reacts with the alkyne or alkene (preferably a strained alkyne or alkene) group of the PEG fragment. However, at higher pH, the Comparative Example teaches that a side product was formed, characterized as a hydroamination reaction between the nitrogen atoms of the LPEI fragment and the alkene or alkyne. Without wishing to be bound by theory, the present invention teaches that an LPEI fragment (e.g., comprising a terminal azide) can be chemoselectively bonded to a PEG fragment (e.g., comprising an activated, preferably strained alkene or alkyne), at a pH below about 5, preferably about 4 or below.

Thus, in another aspect, the present invention provides a method of synthesizing a conjugate of Formula I, comprising reacting an LPEI fragment comprising a thiol with a PEG fragment comprising an alkene.

In another aspect, the present invention provides a method of synthesizing a conjugate as described and defined herein, and preferably a method of synthesizing a conjugate of Formula I, wherein the method comprises reacting the omega terminus of a linear polyethyleneimine fragment with a first terminal end of a polyethylene glycol fragment, wherein said reaction occurs at a pH below about 5, preferably 4 or below, and wherein preferably said omega terminus of said linear polyethyleneimine fragment comprises an azide, and wherein said first terminal end of said polyethylene glycol fragment comprises an alkene or an alkyne, and wherein said reaction is between said azide and said alkene or an alkyne.

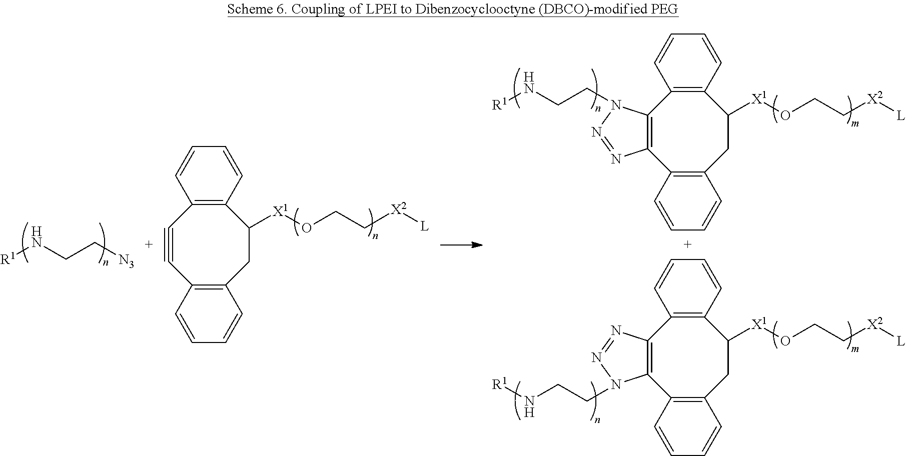

As shown above in Scheme 6, in some embodiments PEG can be modified to include a strained alkyne group such DBCO. Treatment of the DBCO-modified PEG in solution with an azide-modified LPEI results in a [3+2]cycloaddition of the azide to the alkyne of DBCO to produce a 1, 2, 3 triazole. One of skill in the art will appreciate that the reaction shown above in Scheme 6 can produce triazole adducts with different regiochemistries as shown above. The variables m and n represent the number of repeating PEG and LPEI units as described herein.

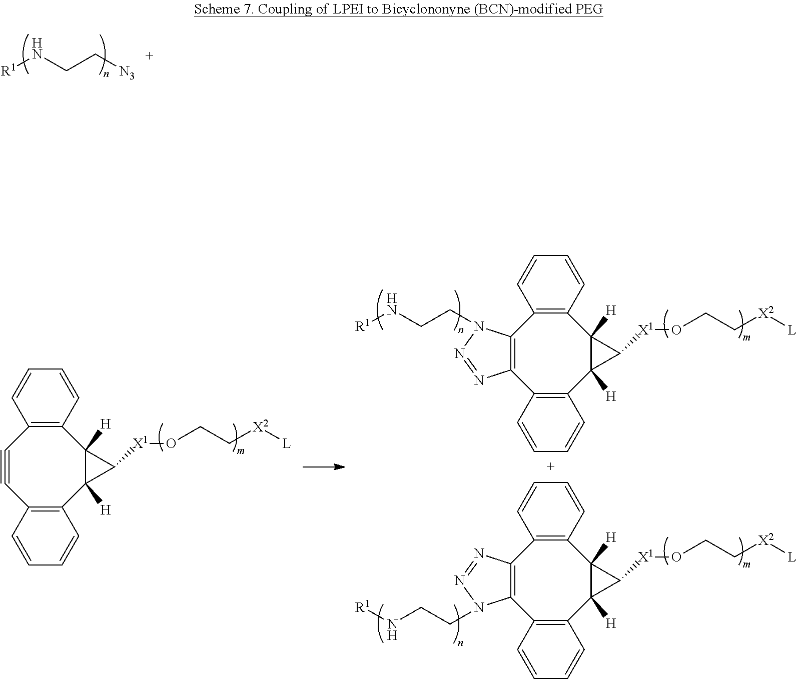

As shown above in Scheme 7, in some embodiments PEG can be modified to include a strained alkyne group such bicyclononyne (BCN). Treatment of the BCN-modified PEG in solution with an azide-modified LPEI results in a [3+2]cycloaddition of the azide to the alkyne of BCN to produce a 1, 2, 3 triazole. One of skill in the art will appreciate that the reaction shown above in Scheme 7 can produce triazole adducts with different regiochemistries as shown above. The variables m and n represent the number of repeating PEG and LPEI units as described herein.

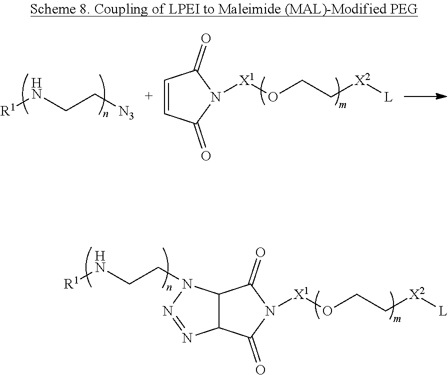

As shown above in Scheme 8, in some embodiments PEG will be modified to include an alkene group such as maleimide (MAL). Treatment of the MAL-modified PEG in solution with an azide-modified LPEI will result in a [3+2]cycloaddition of the azide to the alkene of MAL to produce a 4,5-dihydro-1H-[1,2,3]triazole. The variables m and n will represent the number of repeating PEG and LPEI units as described herein.

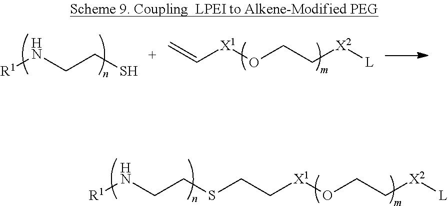

As shown above in Scheme 9, in some embodiments PEG can be modified to include a terminal alkene group and LPEI can be modified to include a terminal thiol group. Treatment of the thiol-modified LPEI in solution with an alkene-modified PEG can result in a thiol-ene reaction to produce a thioether. The variables m and n will represent the number of repeating PEG and LPEI units as described herein.

X¹ and X² Linking Moieties

In some embodiments, the PEG fragments of the conjugates of the present invention can be connected to alkene or alkyne groups and/or targeting fragments by covalent linking moieties.

### X¹ Linking Moieties

In some embodiments, PEG fragments of the conjugates of the present invention are connected to an activated (e.g., cyclic) alkene or alkyne group on a terminal end by a linking moiety. For instance, the X¹ linking moiety can be formed as the result of selecting a PEG fragment and an alkene or alkyne group that each contain reactive functional groups that can be combined by well-known chemical reactions. For example, a PEG fragment can be coupled to an activated (e.g., cyclic) alkene or alkyne group by standard means such as peptide coupling (e.g., to form an amide), nucleophilic addition, or other means known to one of skill in the art.

In one aspect, X¹ is a linking moiety of the formula —(Y¹)ₚ—, wherein p is an integer between 1 and 20, and each occurrence of Y¹ is independently selected from a chemical bond, —CR¹“R¹²—, —C(O)—, —O—, —S—, —NR¹³—, an amino acid residue, a divalent phenyl moiety, a divalent carbocyle moiety, a divalent heterocycle moiety, and a divalent heteroaryl moiety, wherein each divalent phenyl or heteroaryl is optionally substituted with one or more R”, and each divalent heterocycle is optionally substituted with one or more R¹⁴; R¹¹, R¹² and R¹³ are independently, at each occurrence, H, —SO₃H, —NH₂, or C₁-C₆ alkyl, wherein each alkyl is optionally substituted with —CO₂H or NH₂; and R¹⁴ is independently, at each occurrence, H, C₁-C₆ alkyl, or oxo, C₆-C₁₀ aryl, or 5 to 8-membered heteroaryl.

In some embodiments, when Y¹ is an amino acid residue, it can be oriented in any direction, i.e., —C(O)—CHR—NH— or —NH—CHR—C(O)—, wherein “R” represents the side-chain of a naturally occurring amino acid.

In some embodiments, the divalent heteroaryl moiety is a divalent heteroaryl group comprising one or more heteroatoms selected from O, N, S, and P, preferably one or two atoms selected from O and N. In some embodiments, the divalent heteroaryl moiety is a divalent furan, pyrrole, imidazole, pyrazole, triazole, pyridine, pyrimidine, pyridazine, pyrazine, thiophene, oxazole, or isoxazole; wherein the divalent heteroaryl is optionally substituted with one or more, preferably one or zero R¹⁴.

In the embodiments below for X¹, unless otherwise specified, a wavy line indicates a bond in any direction, i.e., to a PEG fragment or to the divalent covalent linking moiety (e.g., “Z” or Ring A).

In some embodiments, the divalent heterocycle moiety is a divalent heterocycle group comprising one or more heteroatoms selected from O, N, S, and P, preferably one or two atoms selected from O and N. In some embodiments, the divalent heterocycle moiety is a divalent tetrahydrofuran, pyrrolidine, piperidine, or 4,5-Dihydro-isoxazole, each optionally substituted with one or more R¹⁴. In some preferred embodiments, the divalent heterocycle moiety is a succinimide. In some preferred embodiments, two Y¹ can combine to form a linking moiety or partial linking moiety of the formula

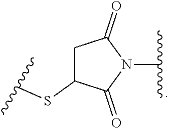 [SMILES: *SC1CC(=O)N(*)C1=O]

In a further preferred embodiment, two Y¹ can combine to form a linking moiety or partial linking moiety of the formula

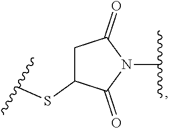 [SMILES: *SC1CC(=O)N(*)C1=O]

wherein the wavy line next to the sulfur represents the direction of connectivity towards the targeting fragment.

In a further preferred embodiment, Y¹ can comprise a linking moiety or partial linking moiety of the formula:

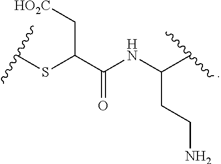 [SMILES: *SC(CC(=O)O)C(=O)NC(*)CCN]

In a further preferred embodiment, Y¹ can comprise a linking moiety or partial linking moiety of the formula:

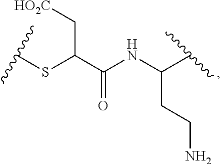 [SMILES: *SC(CC(=O)O)C(=O)NC(*)CCN]

wherein the wavy line next to the sulfur represents the direction of connectivity towards the targeting fragment.

In some embodiments, X¹ is a linking moiety of the formula —(Y¹)ₚ—, wherein p is an integer between 1 and 8, and each occurrence of Y¹ is independently selected from a chemical bond, —CHR¹¹—, —C(O)—, —O—, —S—, —NH—, —C₆H₄—,

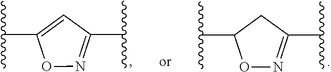 [SMILES: *c1cc(*)on1 ; *C1=NOC(*)C1]

In some embodiments, X¹ is a linking moiety of the formula —(Y¹)ₚ—, wherein p is an integer between 1 and 8, and each occurrence of Y¹ is independently selected from a chemical bond, —CH₂—, —C(O)—, —O—, —S—, —NH—, —C₆H₄—,

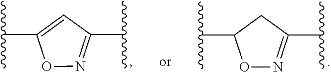 [SMILES: *c1cc(*)on1 ; *C1=NOC(*)C1]

In some embodiments, X¹ is a linking moiety of the formula —(Y¹)ₚ—, wherein p is an integer between 1 and 8, and each occurrence of Y¹ is independently selected from a chemical bond, —CH₂—, —C(O)—, —O—, —S—, —NH—,

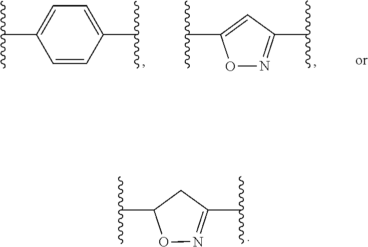 [SMILES: *c1ccc(*)cc1 ; *c1cc(*)on1 ; *C1=NOC(*)C1]

In some embodiments, X¹ is a linking moiety of the formula —(Y¹)ₚ—, wherein p is an integer between 1 and 8, and each occurrence of Y¹ is independently selected from a chemical bond, —CH₂—, —C(O)—, —O—, —NH—,

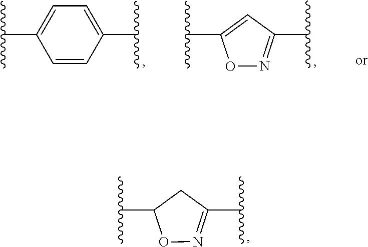

wherein Y¹ is only —NH—when it is adjacent to a —C(O)—group to form a carbamate or amide.

In some embodiments, X¹ is

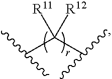

wherein r is an integer between 1 and 8, preferably between 1 and 4, more preferably between 1 and 2; and wherein R¹¹ and R¹² are independently —H or C₁-C₆ alkyl, preferably —H or C₁-C₂ alkyl, more preferably —H.

In some embodiments, X¹ is

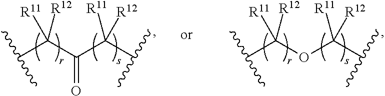

wherein r and s are each independently an integer between 0 and 4, preferably between 1 and 3, more preferably between 1 and 2; and wherein the sum of r and s is less than or equal to 7; and wherein R¹¹ and R¹² are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “s” is a bond to the PEG fragment —[OCH₂—CH₂]ₘ—.

In some embodiments, X¹ is

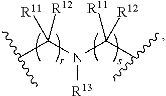

wherein s and t are each independently an integer between 0 and 4, preferably between 1 and 3, more preferably between 1 and 2; and wherein the sum of r and s is less than or equal to 7; and wherein R¹¹, R¹², and R¹³ are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “s” is a bond to the PEG fragment —[OCH₂—CH₂]ₘ—.

In some embodiments, X¹ is

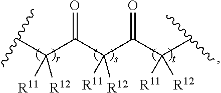

wherein r is an integer between 0 and 3, preferably between 1 and 3, more preferably between 1 and 2; s and t are each independently an integer between 0 and 2, preferably 0 and 1; wherein the sum of r, s, and t is less than or equal to 6; and wherein R¹¹ and R¹² are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “t” is a bond to the PEG fragment —[OCH₂—CH₂]ₘ—.

In some embodiments, X¹ is

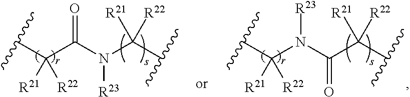

wherein r and s are each independently an integer between 0 and 4, preferably between 1 and 3, more preferably between 1 and 2; and wherein the sum of r and s is less than or equal to 6; and wherein R¹¹, R¹² and R¹³ are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “s” is a bond to the PEG fragment —[OCH₂—CH₂]ₘ—.

In some embodiments, X¹ is

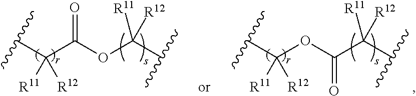

wherein r and s are each independently an integer between 0 and 4, preferably between 1 and 3, more preferably between 1 and 2; and wherein the sum of r and s is less than or equal to 6; and wherein R¹¹, R¹² and R¹³ are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “s” is a bond to the PEG fragment —[OCH₂—CH₂]ₘ—.

In some embodiments, X¹ is

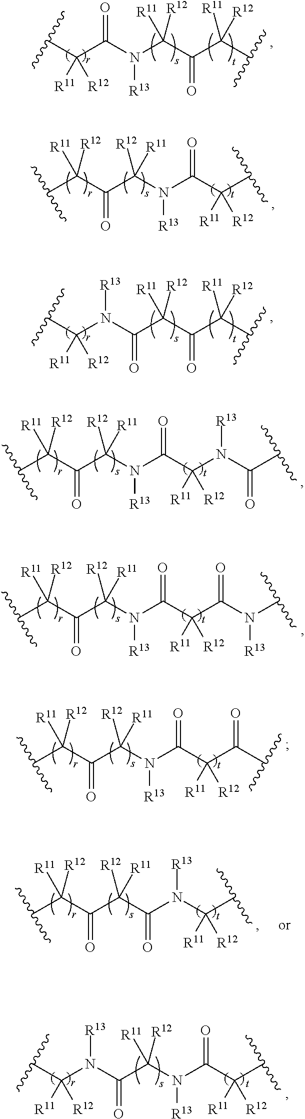

wherein r and t are each an integer between 0 and 3 and s is an integer between 0 and 3; preferably wherein r is 0, s is 2 or 3, and t is 2; wherein the sum of r, s and t is less than or equal to 5; and wherein R¹¹, R¹² and R¹³ are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “t” is a bond to the PEG fragment —[OCH₂—CH₂]ₘ—.

In some embodiments, X^{t} is

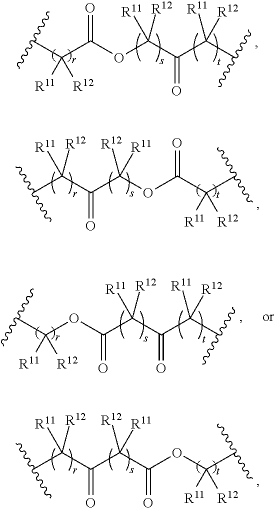

wherein r and t are each an integer between 0 and 3; s is an integer between 0 and 3; wherein the sum or r, s and t is less than or equal to 5; and wherein R¹¹ and R¹² are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “t” is a bond to the PEG fragment —[OCH₂—CH₂]ₘ—.

In some embodiments, X¹ is

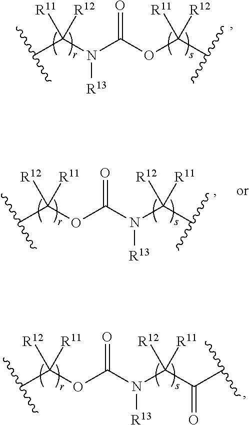

wherein r and s are each independently an integer between 0 and 3, preferably between 0 and 2; wherein the sum of r and s is less than or equal to 5; and wherein R¹¹, R¹² and R¹³ are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the integer “s” is a bond to the PEG fragment —[OCH₂—CH₂]ₘ—.

In some embodiments, X¹ is

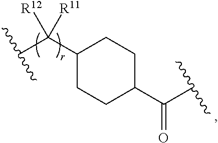

wherein r is independently an integer between 0 and 4, preferably between 0 and 2, more preferably between 1 and 2; and wherein R¹¹, and R¹² are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the carbonyl group is a bond to the PEG fragment —[OCH₂—CH₂]ₘ—.

In some embodiments, X¹ is

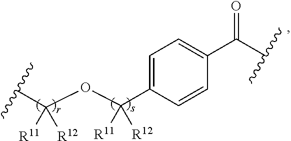

wherein r and s are each independently an integer between 0 and 4, preferably between 0 and 2, more preferably between 1 and 2; wherein the sum of r and s is less than or equal to 5; and wherein R¹¹, and R¹² are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the carbonyl group is a bond to the PEG fragment —[OCH₂—CH₂]ₘ—.

In some embodiments, X¹ is

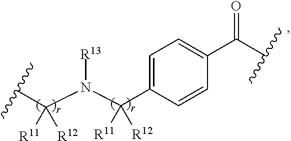

wherein r and s are each independently an integer between 0 and 4, preferably between 0 and 2; wherein the sum of r and s is less than or equal to 5; and wherein R¹¹, R¹² and R¹³ are independently —H or C₁-C₆ alkyl, preferably —H or C₁-C₂ alkyl, more preferably —H. Preferably the wavy line nearest to the integer “r” is a bond to the divalent covalent linking moiety (e.g., “Z” or Ring A) and the wavy line nearest to the carbonyl group is a bond to the PEG fragment —[OCH₂—CH₂]ₘ—.

In some preferred embodiments, X¹ is selected from:

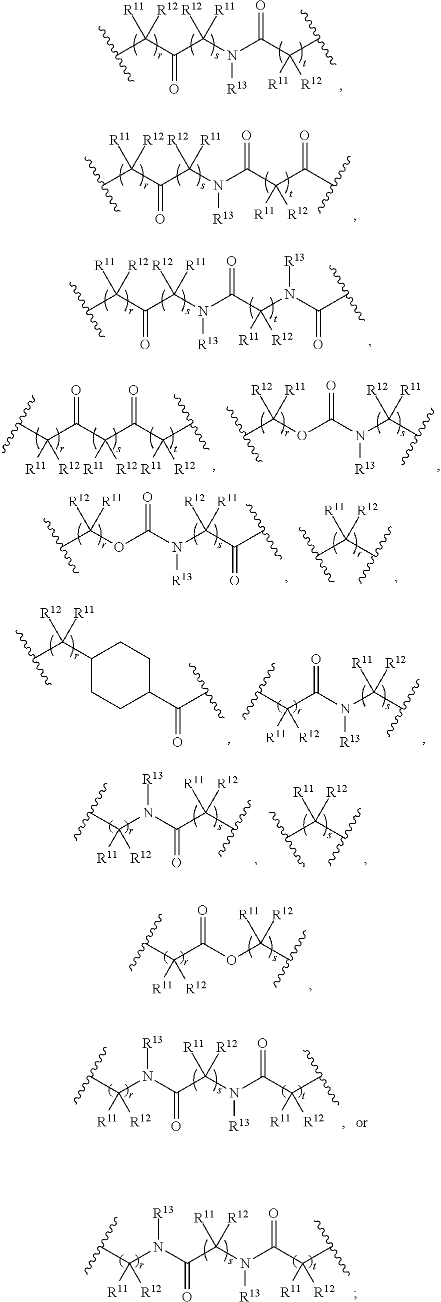

wherein
r is independently, at each occurrence, 0-6, preferably 0, 1, 2, or 5;
s is independently, at each occurrence, 0-6, preferably 0, 2, 4;
t is independently, at each occurrence, 0-6, preferably 0, 1, 2, 4;
R¹¹ and R¹² are independently, at each occurrence, selected from —H, —C₁-C₂ alkyl, —SO₃H, and —NH₂; more preferably —H, —SO₃H, and —NH₂; yet more preferably —H; and
R¹³ is —H. Preferably the wavy line nearest to the integer “r” is a bond to the divalent covalent linking moiety (e.g., “Z” or Ring A) and the wavy line nearest to the integer “s” or “t” or carbonyl group is a bond to the PEG fragment —[OCH₂—CH₂]ₘ—.

In some preferred embodiments, X¹ is selected from:

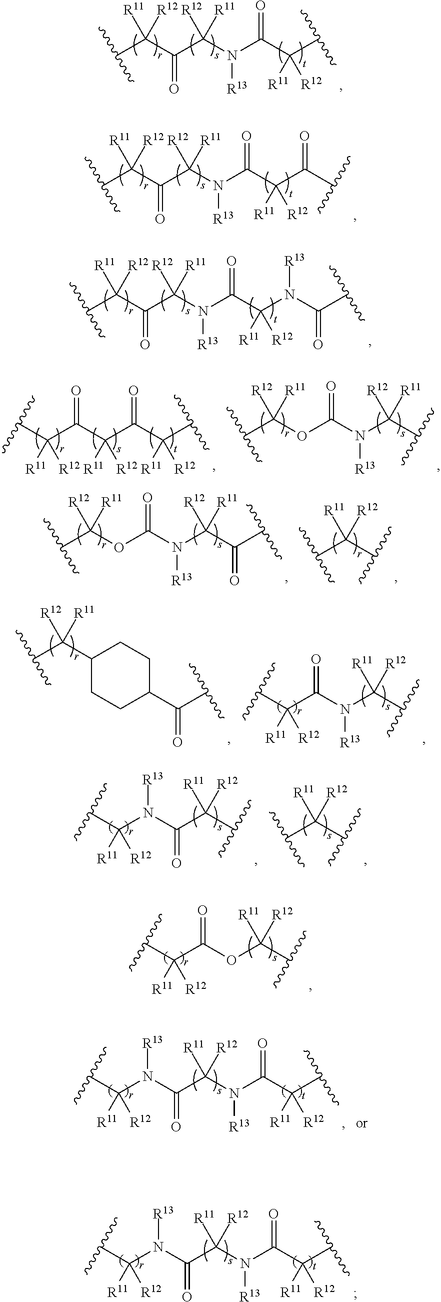

wherein:
r is independently, at each occurrence, 0-6, preferably 0, 1, 2, or 5;
s is independently, at each occurrence, 0-6, preferably 0, 2, 4;
t is independently, at each occurrence, 0-6, preferably 0, 1, 2, 4;
R¹¹ and R¹² are independently, at each occurrence, selected from —H and —C₁-C₂ alkyl, preferably —H; and
R¹³ is —H. Preferably the wavy line nearest to the integer “r” is a bond to the divalent covalent linking moiety (e.g., “Z” or Ring A) and the wavy line nearest to the integer “s” or “t” or carbonyl group is a bond to the PEG fragment —[OCH₂—CH₂]ₘ—.

In some preferred embodiments, X¹ is a group selected from:

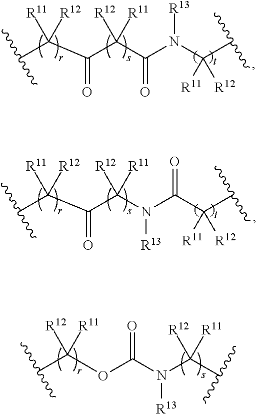

wherein:
r is independently, at each occurrence, 0-6, preferably 0, 1, 2, or 5; more preferably 0;
s is independently, at each occurrence, 0-6, preferably 0, 2, 3, or 4; more preferably 2 or 3;
t is independently, at each occurrence, 0-6, preferably 0, 1, 2, 4; more preferably 2;
R¹¹ and R¹² are independently, at each occurrence, selected from —H and —C₁-C₂ alkyl, preferably —H; and
R¹³ is —H. Preferably the wavy line nearest to the integer “r” is a bond to the divalent covalent linking moiety (e.g., “Z” or Ring A) and the wavy line nearest to the integer “s” or “t” group is a bond to the PEG fragment —[OCH₂—CH₂]ₘ—.

In some preferred embodiments, X¹ is selected from:

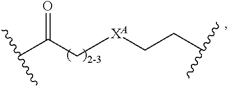

wherein X^{A} is —NHC(O)— or —C(O)NH—; and

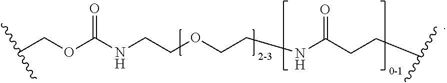

Preferably the wavy line on the left side is a bond to the divalent covalent linking moiety (e.g., “Z” or Ring A) and the wavy line on the right side is a bond to the PEG fragment —[OCH₂—CH₂]ₘ—.

In some preferred embodiments, X¹ is selected from:

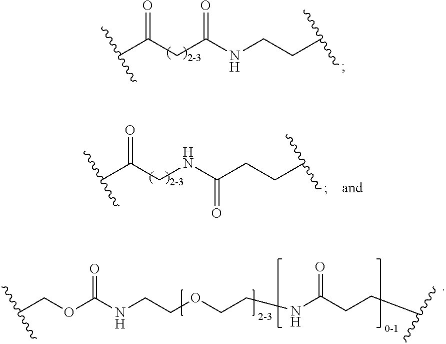

Preferably the wavy line on the left side is a bond to the divalent covalent linking moiety (e.g., “Z” or Ring A) and the wavy line on the right side is a bond to the PEG fragment —[OCH₂—CH₂]ₘ—.

In some preferred embodiments, X¹ is selected from:

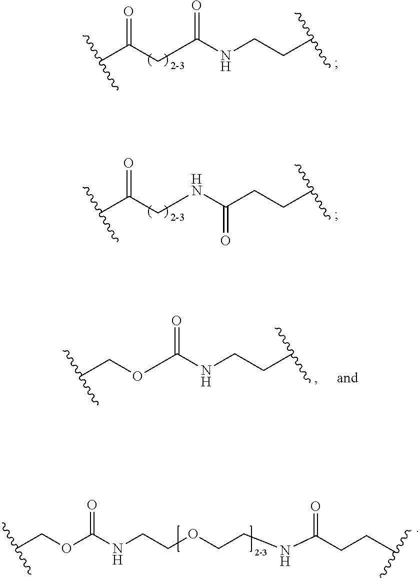

Preferably the wavy line on the left side is a bond to the divalent covalent linking moiety (e.g., “Z” or Ring A) and the wavy line on the right side is a bond to the PEG fragment —[OCH₂—CH₂]ₘ—.

In some embodiments, X¹ is selected from:

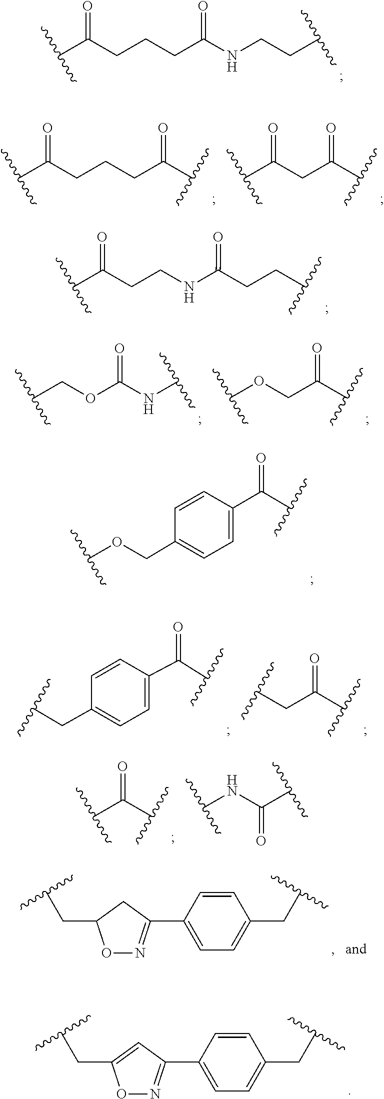

Preferably the wavy line on the left side is a bond to the divalent covalent linking moiety (e.g., “Z” or Ring A) and the wavy line on the right side is a bond to the PEG fragment —[OCH₂—CH₂]ₘ—.

In some preferred embodiments, X¹ is selected from:

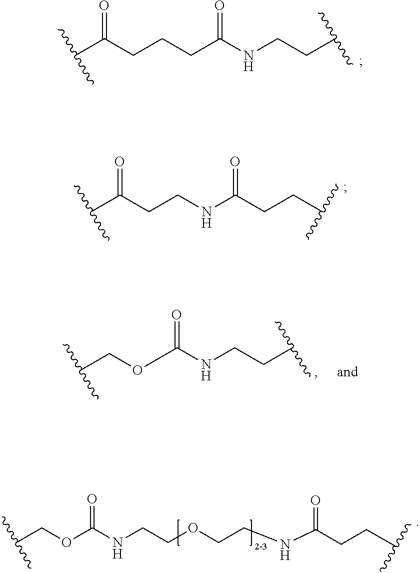

Preferably the wavy line on the left side is a bond to the divalent covalent linking moiety (e.g., “Z” or Ring A) and the wavy line on the right side is a bond to the PEG fragment —[OCH₂—CH₂]ₘ—.

In some preferred embodiments, X¹ is —(CH₂)₁₋₆—; preferably X¹ is —(CH₂)₂₋₄—; more preferably X¹ is —(CH₂)₂—.

### X² Linking Moieties

In some embodiments, PEG fragments of the conjugates of the present invention are connected to a targeting fragment on a terminal end by a linking moiety. For instance, the X² linking moiety can be formed as the result of selecting a PEG fragment and a targeting fragment that each contain reactive functional groups that can be combined by well-known chemical reactions. For example, a PEG fragment can be coupled to a targeting group by standard means such as peptide coupling (e.g., to form an amide), nucleophilic addition, or other means known to one of skill in the art.

In one aspect, X² is a linking moiety of the formula —(Y²)_{q}—, wherein q is an integer between 1 and 50, and each occurrence of Y² is independently selected from a chemical bond, —CR²¹R²²—, NR²³—, —O—, —S—, —C(O)—, an amino acid residue, a divalent phenyl moiety, a divalent carbocyle moiety, a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴;
R²¹, R²², and R²³ are each independently, at each occurrence, —H, —SO₃H, —NH₂, —CO₂H, or C₁-C₆ alkyl, wherein each C₁-C₆ alkyl is optionally substituted with one or more —OH, oxo, —CO₂H, —NH₂, C₆-C₁₀ aryl, or 5 to 8-membered heteroaryl;
R²⁴ is independently, at each occurrence, —H, —CO₂H, C₁-C₆ alkyl, or oxo.

In some embodiments, R²¹, R²² and R²³ are each independently, at each occurrence, —H, —CO₂H, or C₁-C₆ alkyl. In some embodiments, R²¹, R²² and R²³ are each, independently —H or C₁-C₄ alkyl, preferably C₁-C₂ alkyl.

In some embodiments, R²¹, R²², R²³, and R²⁴ are —H.

In some embodiments, R²⁴ is independently —H, C₁-C₆ alkyl, or oxo.

In some embodiments, the divalent heteroaryl moiety is a divalent heteroaryl group comprising one or more heteroatoms selected from O, N, S, and P, preferably one or two atoms selected from O and N. In some embodiments, the divalent heteroaryl moiety is a divalent furan, pyrrole, imidazole, pyrazole, triazole, pyridine, pyrimidine, pyridazine, pyrazine, thiophene, oxazole, or isoxazole; wherein the divalent heteroaryl is optionally substituted with one or more, preferably one or zero R²¹.

In the embodiments below for X², unless otherwise specified, a wavy line indicates a bond in any direction, i.e., to a PEG fragment (—[OCH₂CH₂]ₘ—) or to a targeting fragment (i.e., “L”).

In some embodiments, the divalent heterocycle moiety is a divalent heterocycle group comprising one or more heteroatoms selected from O, N, S, and P, preferably one or two atoms selected from O and N. In some embodiments, the divalent heterocycle moiety is a divalent tetrahydrofuran, pyrrolidine, piperidine, or 4,5-dihydro-isoxazole, each optionally substituted with one or more R²⁴. In some preferred embodiments, the divalent heterocycle moiety is a succinimide. In some preferred embodiments, two Y² can combine to form a linking moiety or partial linking moiety of the formula

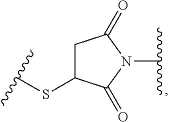 [SMILES: *SC1CC(=O)N(*)C1=O]

In a further preferred embodiment, two Y² can combine to form a linking moiety or partial linking moiety of the formula

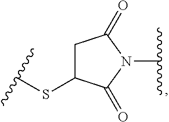 [SMILES: *SC1CC(=O)N(*)C1=O]

wherein the wavy line next to the sulfur represents a bond to the targeting fragment (L) and the wavy line next to the nitrogen represents a bond to the PEG fragment (—[OCH₂—CH₂]ₘ—).

In a further preferred embodiment, two Y² can combine to form a linking moiety or partial linking moiety of the formula

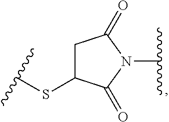 [SMILES: *SC1CC(=O)N(*)C1=O]

wherein the wavy line next to the sulfur represents a bond to the PEG fragment (—[OCH₂—CH₂]ₘ—) and the wavy line next to nitrogen represents a bond to the targeting fragment (L).

In a further preferred embodiment, Y² can comprise a linking moiety or partial linking moiety of the formula:

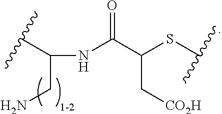

In a further preferred embodiment, Y² can comprise a linking moiety or partial linking moiety of the formula:

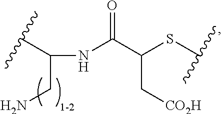

wherein the wavy line next to the sulfur represents the direction of connectivity towards the targeting fragment.

In some embodiments, X² is a linking moiety of the formula —(Y²)_{q}—, wherein q is an integer between 1 and 40, and each occurrence of Y² is independently selected from a chemical bond, —CR²¹R²²—, NH—, —O—, —S—, —C(O)—, an amino acid residue, and

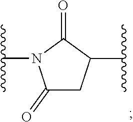 [SMILES: *C1CC(=O)N(*)C1=O]

and R²¹ and R²² are independently, at each occurrence, —H, —CO₂H, or C₁-C₆ alkyl, wherein each C₁-C₆ alkyl is optionally substituted with one or more —OH, oxo, C₆-C₁₀ aryl, or 5 to 8-membered heteroaryl.

In some embodiments, X² is a linking moiety of the formula —(Y²)_{q}—, wherein q is an integer between 1 and 40, and each occurrence of Y² is independently selected from a chemical bond, —CHR²¹—, NH—, —O—, —S—, —C(O)—, an amino acid residue, and

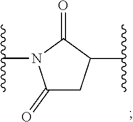 [SMILES: *C1CC(=O)N(*)C1=O]

and
R²¹ is independently, at each occurrence, —H, —CO₂H, or C₁-C₄ alkyl (preferably C₁ alkyl), wherein each C₁-C₄ alkyl is optionally substituted with one or more C₆-C₁₀ aryl or 5 to 8-membered heteroaryl.

In some embodiments, X² is a linking moiety of the formula —(Y²)_{q}—, wherein q is an integer between 1 and 40, and each occurrence of Y² is independently selected from a chemical bond, —CHR²¹—, —NH—, —O—, —S—, —C(O)—, an amino acid residue, and

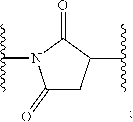 [SMILES: *C1CC(=O)N(*)C1=O]

and
R²¹ is independently, at each occurrence, —H, —CO₂H, or C₁-C₄ alkyl (preferably C₁ alkyl), wherein each C₁-C₄ alkyl is optionally substituted with one or more C₆-C₁₀ aryl or 5 to 8-membered heteroaryl.

In some embodiments, X² is a linking moiety of the formula —(Y²)_{q}—, wherein q is an integer between 1 and 40, and each occurrence of Y² is independently selected from a chemical bond, —CHR²¹—, —NH—, —O—, —S—, —C(O)—, an amino acid residue, and

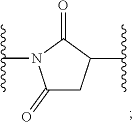 [SMILES: *C1CC(=O)N(*)C1=O]

and
R²¹ is independently, at each occurrence, —H, —CO₂H, or C₁-C₃ alkyl (preferably C₁ alkyl), wherein each C₁-C₃ alkyl is optionally substituted with one or more phenyl or indole.

In some embodiments, X² is a linking moiety of the formula —(Y²)_{q}—, wherein q is an integer between 1 and 40, and each occurrence of Y² is independently selected from a chemical bond, —CHR²¹—, —NH—, —O—, —S—, —C(O)—, an amino acid residue, and

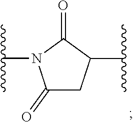 [SMILES: *C1CC(=O)N(*)C1=O]

and
R²¹ is independently, at each occurrence, —H, —CO₂H, or C₁-C₃ alkyl (preferably C₁ alkyl), wherein each C₁-C₃ alkyl is optionally substituted with one or more phenyl or 3-indole.

In some embodiments, X² is a linking moiety of the formula —(Y²)_{q}—, wherein q is an integer between 1 and 40, and each occurrence of Y² is independently selected from a chemical bond, —CHR²¹—, —NH—, —O—, —S—, —C(O)—, an amino acid residue, and

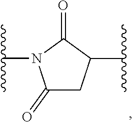

wherein Y² is only —NH—when it is adjacent to a —C(O)—group to form a carbamate or amide; and
R²¹ is independently, at each occurrence, —H, —CO₂H, or C₁-C₃ alkyl (preferably C₁ alkyl), wherein each C₁-C₃ alkyl is optionally substituted with one or more phenyl or 3-indole.

In some embodiments, X² is a linking moiety of the formula —(Y²)_{q}—, wherein q is an integer between 1 and 40, and each occurrence of Y² is independently selected from a chemical bond, —CHR²¹—, —NH—, —O—, —S—, —C(O)—, an amino acid residue, and

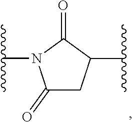

wherein Y² is only —NH—when it is adjacent to a —C(O)—group to form an amide; and
R²¹ is independently, at each occurrence, —H, —CO₂H, or C₁-C₃ alkyl (preferably C₁ alkyl), wherein each C₁-C₃ alkyl is optionally substituted with one or more phenyl or 3-indole.

In some embodiments, when Y² is an amino acid residue, Y² represents a naturally occurring, L- amino acid residue. When Y² is an amino acid residue, it can be oriented in any direction, i.e., —C(O)—CHR—NH— or —NH—CHR—C(O)—, wherein “R” represents the side-chain of a naturally occurring amino acid.

In some embodiments, X² is

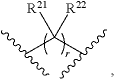

wherein r is an integer between 1 and 8, preferably between 1 and 4, more preferably between 1 and 2; and wherein R²¹ and R²² are independently —H or C₁-C₆ alkyl, preferably —H or C₁-C₂ alkyl, more preferably —H.

In some embodiments, X² is

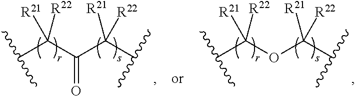

wherein r and s are each independently an integer between 0 and 4, preferably between 1 and 3, more preferably between 1 and 2; and wherein the sum of r and s is less than or equal to 7; and wherein R²¹ and R²² are independently —H or C₁-C₆ alkyl, preferably —H or C₁-C₂ alkyl, more preferably —H.

In some embodiments, X² is

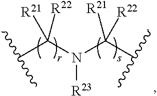

wherein s and t are each independently an integer between 0 and 4, preferably between 1 and 3, more preferably between 1 and 2; and wherein the sum of r and s is less than or equal to 7; and wherein R²¹, R²², and R²³ are independently —H or C₁-C₆ alkyl, preferably —H or C₁-C₂ alkyl, more preferably —H.

In some embodiments, X² is

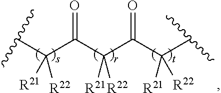

wherein r is an integer between 0 and 3, preferably between 1 and 3, more preferably between 1 and 2; s and t are each independently an integer between 0 and 2, preferably 0 and 1; wherein the sum of r, s, and t is less than or equal to 6; and wherein R²¹ and R²² are independently —H or C₁-C₆ alkyl, preferably —H or C₁-C₂ alkyl, more preferably —H.

In some embodiments, X² is

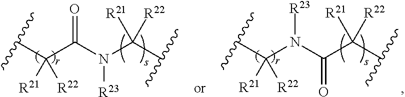

wherein r and s are each independently an integer between 0 and 4, preferably between 1 and 3, more preferably between 1 and 2; and wherein the sum of r and s is less than or equal to 6; and wherein R²¹, R²² and R²³ are independently —H or C₁-C₆ alkyl, preferably —H or C₁-C₂ alkyl, more preferably —H. Preferably the wavy line nearest to the integer “r” is a bond to the PEG fragment (—[OCH₂—CH₂]ₘ—) and the wavy line nearest to the integer “s” is a bond to the targeting fragment (L).

In some embodiments, X² is

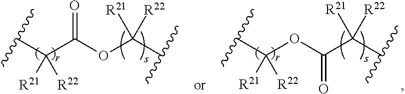

wherein r and s are each independently an integer between 0 and 4, preferably between 1 and 3, more preferably between 1 and 2; and wherein the sum of r and s is less than or equal to 6; and wherein R²¹, R²² and R²³ are independently —H or C₁-C₆ alkyl, preferably —H or C₁-C₂ alkyl, more preferably —H. Preferably the wavy line nearest to the integer “r” is a bond to the PEG fragment (—[OCH₂—CH₂]ₘ—) and the wavy line nearest to the integer “s” is a bond to the targeting fragment (L).

In some embodiments, X² is

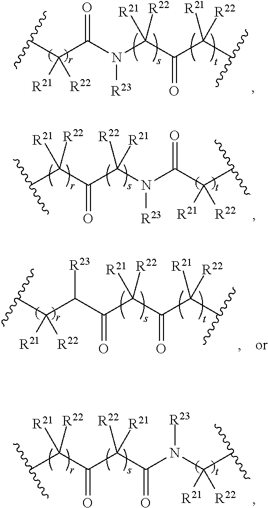

wherein r and t are each an integer between 0 and 3 and s is an integer between 0 and 3; preferably wherein r is 0, s is 2 or 3, and t is 2; wherein the sum of r, s and t is less than or equal to 5; and wherein R²¹, R²² and R²³ are independently —H or C₁-C₆ alkyl, preferably —H or C₁-C₂ alkyl, more preferably —H. Preferably the wavy line nearest to the integer “r” is a bond to the PEG fragment (—[OCH₂—CH₂]ₘ—) and the wavy line nearest to the integer “t” is a bond to the targeting fragment (L).

In some embodiments, X² is

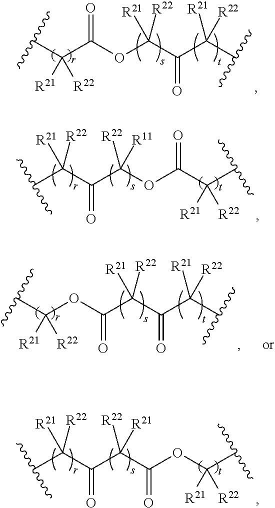

wherein r and t are each an integer between 0 and 3; s is an integer between 0 and 3; wherein the sum or r, s and t is less than or equal to 5; and wherein R²¹ and R²² are independently —H or C₁-C₆ alkyl, preferably —H or C₁-C₂ alkyl, more preferably —H. Preferably the wavy line nearest to the integer “r” is a bond to the PEG fragment (—[OCH₂—CH₂]ₘ—) and the wavy line nearest to the integer “t” is a bond to the targeting fragment (L).

In some embodiments, X² is

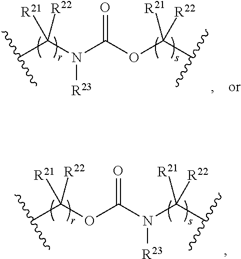

wherein r and s are each independently an integer between 0 and 3, preferably between 0 and 2; wherein the sum of r and s is less than or equal to 5; and wherein R²¹, R²² and R²³ are independently —H or C₁-C₆ alkyl, preferably —H or C₁-C₂ alkyl, more preferably —H. Preferably the wavy line nearest to the integer “r” is a bond to the PEG fragment (—[OCH₂—CH₂]ₘ—) and the wavy line nearest to the integer “s” is a bond to the targeting fragment (L).

In some embodiments, X² is

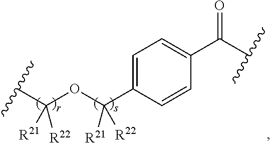

wherein r and s are each independently an integer between 0 and 4, preferably between 0 and 2, more preferably between 1 and 2; wherein the sum of r and s is less than or equal to 5; and wherein R²¹, and R²² are independently —H or C₁-C₆ alkyl, preferably —H or C₁-C₂ alkyl, more preferably —H. Preferably the wavy line nearest to the integer “r” is a bond to the PEG fragment (—[OCH₂—CH₂]ₘ—) and the wavy line nearest to the carbonyl group is a bond to the targeting fragment (L).

In some embodiments, X² is

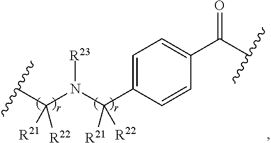

wherein r and s are each independently an integer between 0 and 4, preferably between 0 and 2; wherein the sum of r and s is less than or equal to 5; and wherein R²¹, R²² and R²³ are independently —H or C₁-C₆ alkyl, preferably —H or C₁-C₂ alkyl, more preferably —H. Preferably the wavy line nearest to the integer ‘r’ is a bond to the PEG fragment (—[OCH₂—CH₂]ₘ—) and the wavy line nearest to the carbonyl group is a bond to the targeting fragment (L).

In some embodiments, X² is selected from:

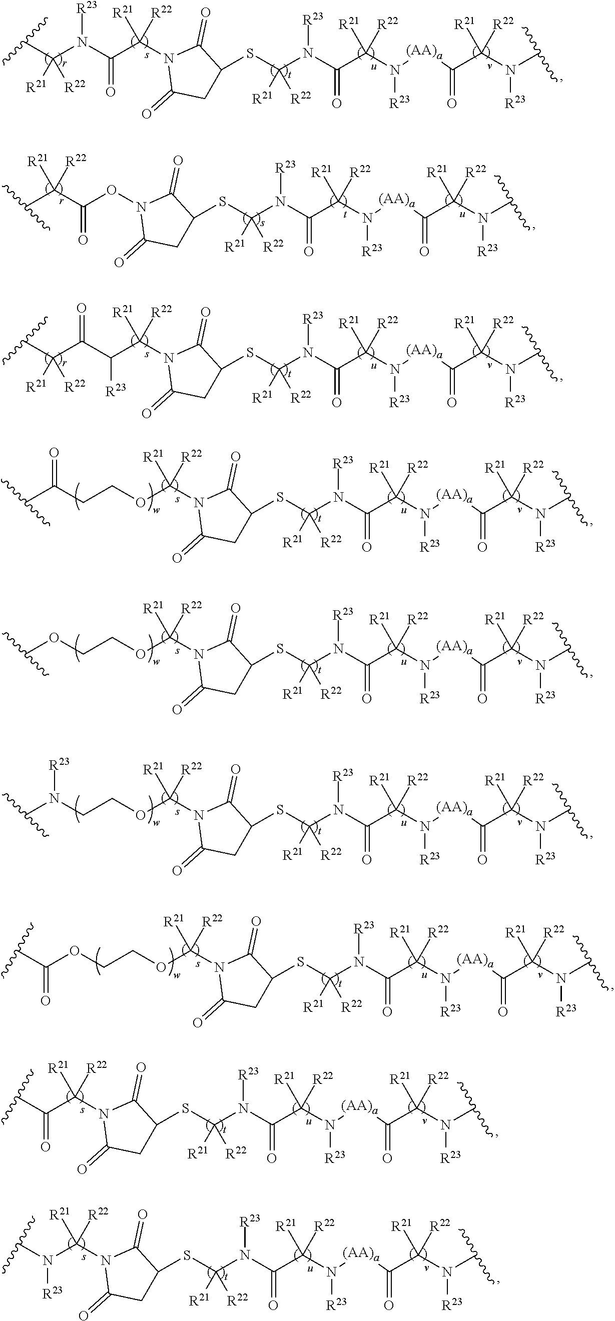

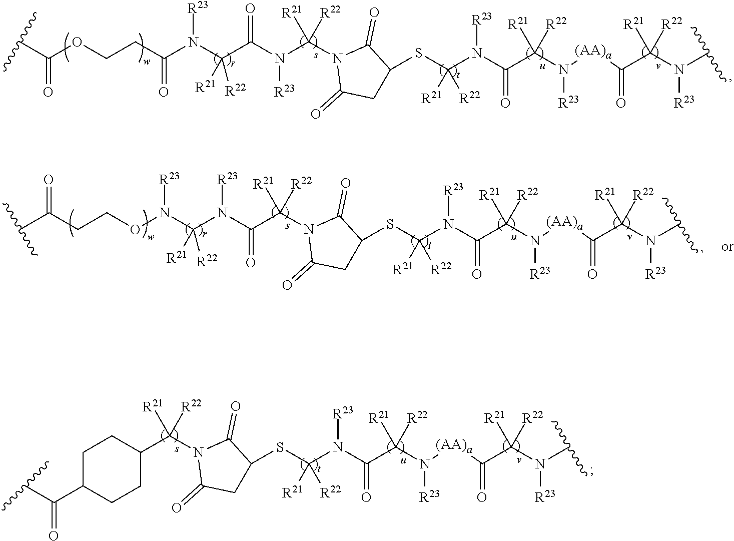

wherein r, s, t and u are each independently an integer between 0 and 6, preferably between 0 and 4: v is an integer between 0 and 10; w is an integer between 0 and 10;
AA is an amino acid residue, preferably a naturally occurring amino acid residue; yet more preferably wherein AA is an amino acid selected from Arg, His, Lys, Asp, Glu, Ser, Thr, Asn, Gln, Cys, Sec, Gly, Pro, Ala, Val, Ile, Leu, Met, Phe, Tyr, and Trp;
a is an integer between 0 and 10, preferably between 0 and 6; more preferably between 0 and 4;
and wherein R²¹, R²² and R²³ are independently —H, C₁-C₆ alkyl or (—COOH), preferably —H, C₁-C₂ alkyl or (—COOH), more preferably —H or (—COOH). Preferably the wavy line on the left side is a bond to the PEG fragment (—[OCH₂—CH₂]ₘ—) and the wavy line on the right side is a bond to the targeting fragment (L).

In some preferred embodiments, (AA)ₐ comprises a tri-peptide selected from Trp-Trp-Gly or Trp-Gly-Phe. In some preferred embodiments, (AA)ₐ is Trp-Trp-Gly-Phe (SEQ ID NO:2).

In some embodiments, X² is selected from:

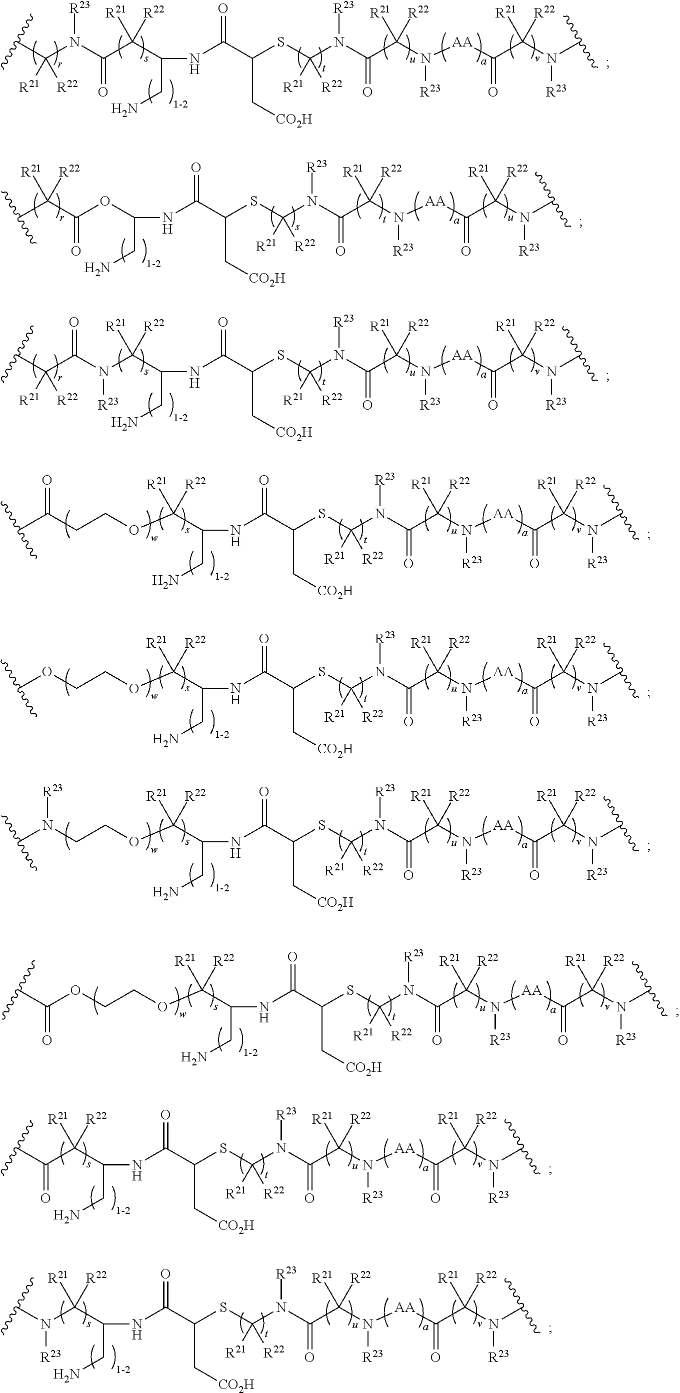

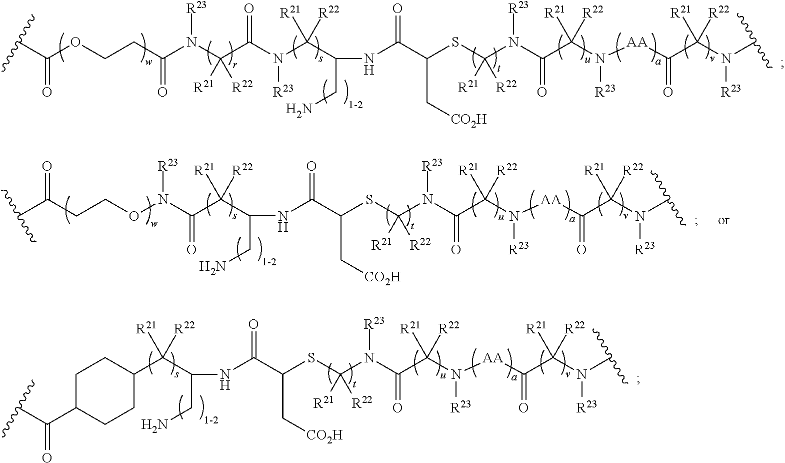

wherein r, s, t and u are each independently an integer between 0 and 6, preferably between 0 and 4; v is an integer between 0 and 10; w is an integer between 0 and 10;
AA is an amino acid residue, preferably a naturally occurring amino acid residue; yet more preferably wherein AA is an an amino acid selected from Arg, His, Lys, Asp, Glu, Ser, Thr, Asn, Gln, Cys, Sec, Gly, Pro, Ala, Val, Ile, Leu, Met, Phe, Tyr, and Trp;
a is an integer between 0 and 10, preferably between 0 and 6; more preferably between 0 and 4;
and wherein R²¹, R²² and R²³ are independently —H, C₁-C₆ alkyl or (—COOH), preferably —H, C₁-C₂ alkyl or (—COOH), more preferably —H or (—COOH). Preferably the wavy line on the left side is a bond to the PEG fragment (—[OCH₂—CH₂]ₘ—) and the wavy line on the right side is a bond to the targeting fragment (L).

In some preferred embodiments, (AA)ₐ is Trp-Trp-Gly-Phe (SEQ ID NO:2).

In some embodiments, X² is selected from:

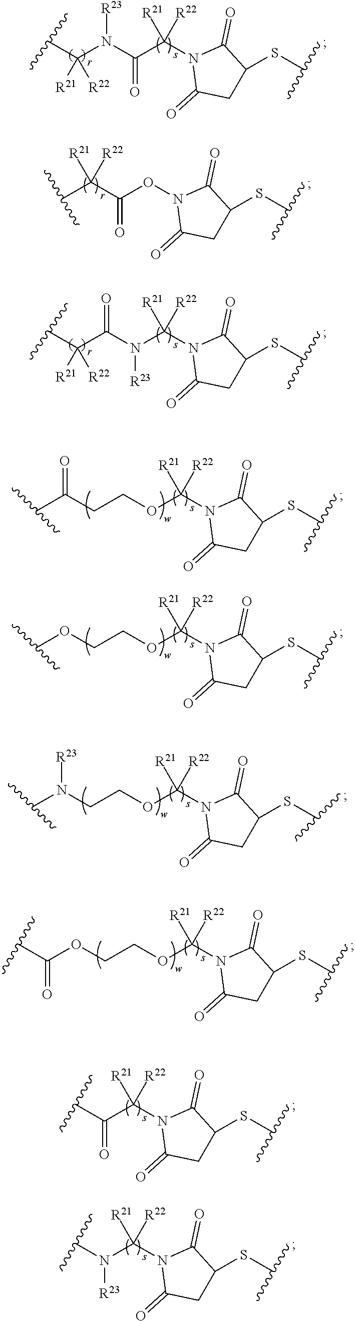

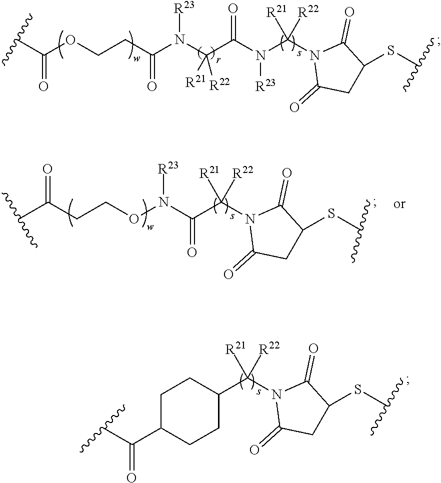

wherein r and s are each independently an integer between 0 and 4, preferably between 0 and 2; w is an integer between 0 and 10;
and wherein R²¹, R²² and R²³ are independently —H or C₁-C₆ alkyl, preferably —H or C₁-C₂ alkyl, more preferably —H. Preferably the wavy line on the left side is a bond to the PEG fragment (—[OCH₂—CH₂]—) and the wavy line on the right side is a bond to the targeting fragment (L).

In some embodiments, X² is selected from:

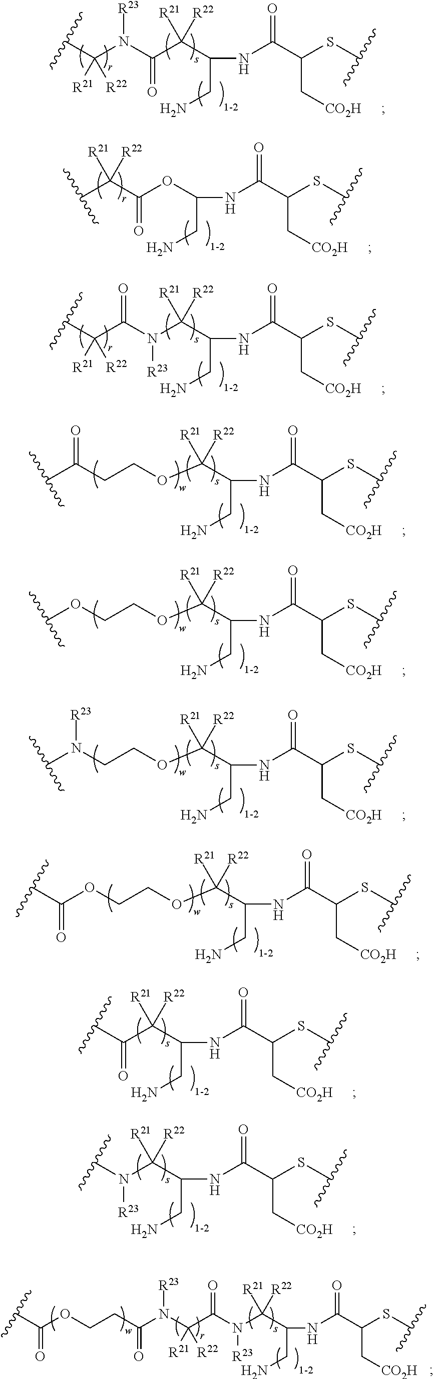

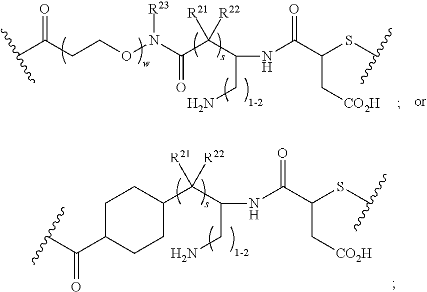

wherein r and s are each independently an integer between 0 and 4, preferably between 0 and 2; w is an integer between 0 and 10;
and wherein R²¹, R²² and R²³ are independently —H or C₁-C₆ alkyl, preferably —H or C₁-C₂ alkyl, more preferably —H. Preferably the wavy line on the left side is a bond to the PEG fragment (—[OCH₂—CH₂]m) and the wavy line on the right side is a bond to the targeting fragment (L).

In some preferred embodiments, X² is selected from:

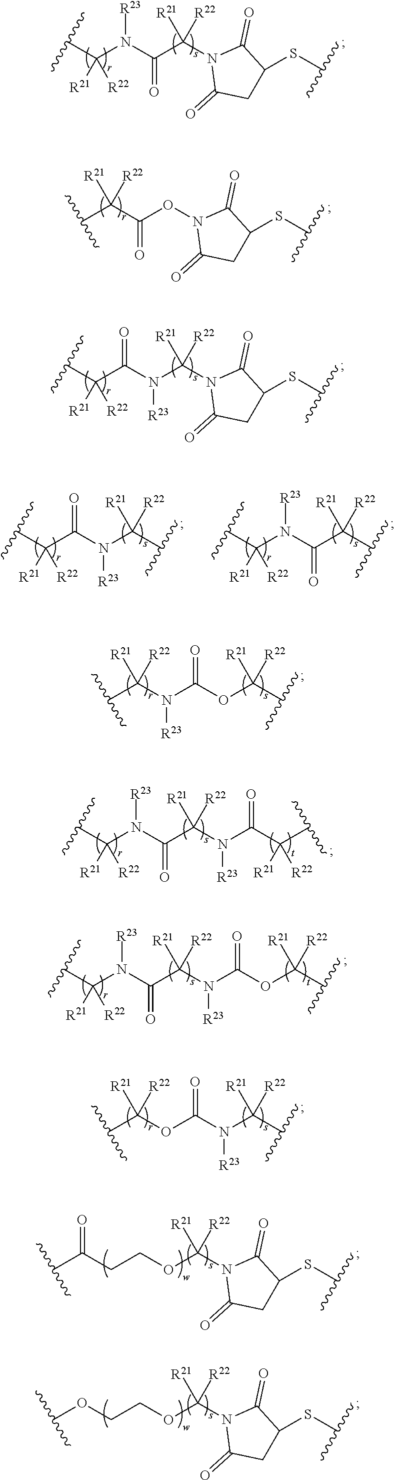

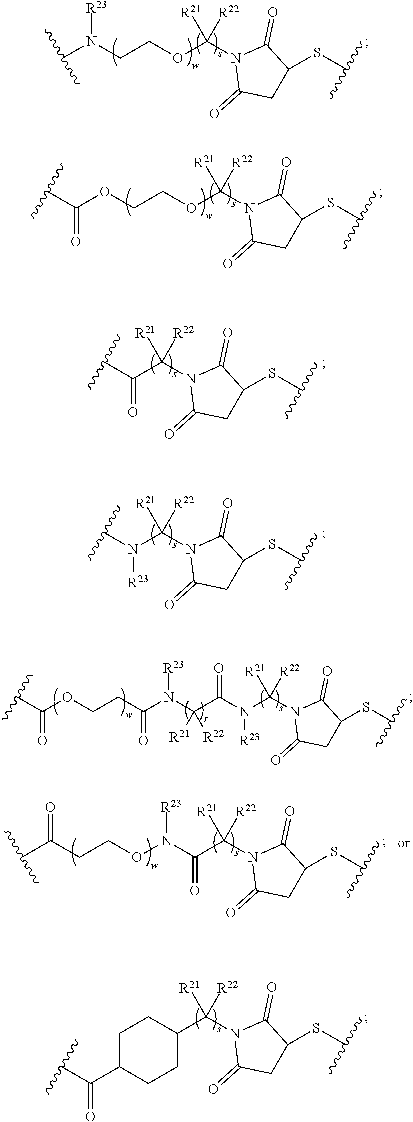

wherein;
r, s, and t, are each independently an integer between 0 and 4, preferably between 0 and 2; w is an integer between 0 and 10;
AA is an amino acid selected from Arg, His, Lys, Asp, Glu, Ser, Thr, Asn, Gln, Cys, Sec, Gly, Pro, Ala, Val, Ile, Leu, Met, Phe, Tyr, and Trp;
a is an integer between 0 and 10, preferably between 0 and 6; more preferably between 0 and 4;
and wherein R²¹, R²² and R²³ are independently —H or C₁-C₆ alkyl, preferably —H or C₁-C₂ alkyl, more preferably —H. Preferably the wavy line on the left side is a bond to the PEG fragment (—[OCH₂—CH₂]m) and the wavy line on the right side is a bond to the targeting fragment (L).

In yet more preferred embodiments, (AA)ₐ is Trp-Trp-Gly-Phe (SEQ ID NO:2).

In some embodiments, X² comprises or alternatively is a urea, a carbamate, a carbonate, or an ester. In preferred embodiments, X² is selected from:

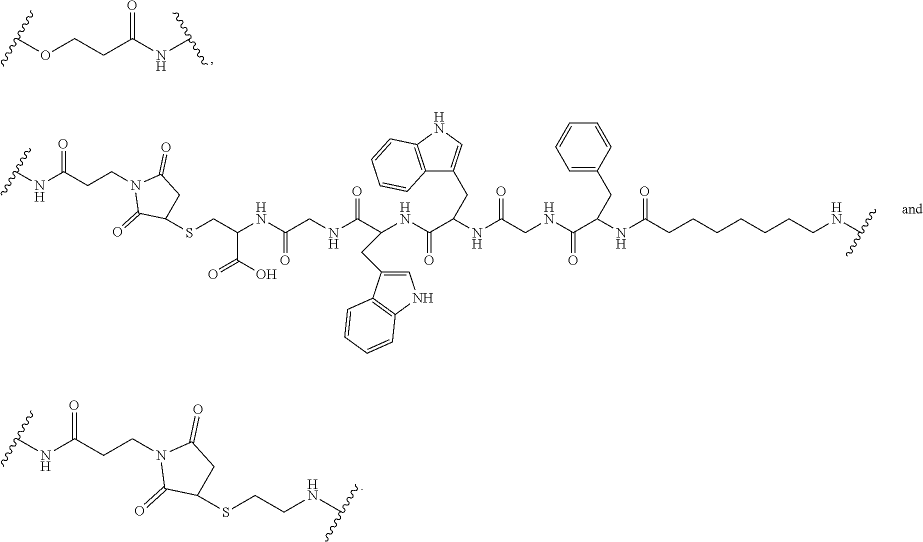 [SMILES: *NC(=O)CCO* ; *NCCCCCCCC(=O)NC(Cc1ccccc1)C(=O)NCC(=O)NC(Cc1c[nH]c2ccccc12)C(=O)NC(Cc1c[nH]c2ccccc12)C(=O)NCC(=O)NC(CSC1CC(=O)N(CCC(=O)N*)C1=O)C(=O)O ; *NCCSC1CC(=O)N(CCC(=O)N*)C1=O]

Preferably the wavy line on the left side is a bond to the PEG fragment (—[OCH₂—CH₂]ₘ) and the wavy line on the right side is a bond to the targeting fragment (L).

In a preferred embodiment said X² is

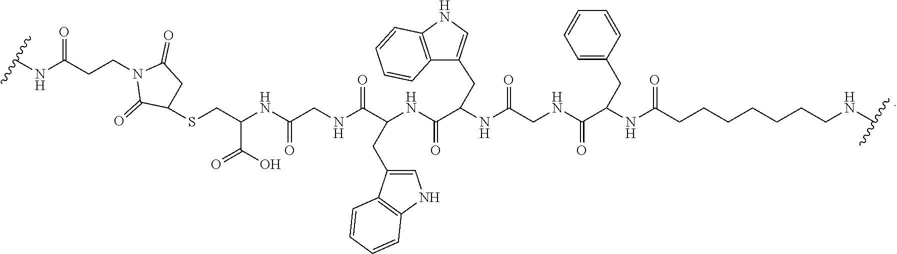 [SMILES: *NCCCCCCCC(=O)NC(Cc1ccccc1)C(=O)NCC(=O)NC(Cc1c[nH]c2ccccc12)C(=O)NC(Cc1c[nH]c2ccccc12)C(=O)NCC(=O)NC(CSC1CC(=O)N(CCC(=O)N*)C1=O)C(=O)O]

Preferably the wavy line on the left side is a bond to the PEG fragment (—[OCH₂—CH₂]ₘ—) and the wavy line on the right side is a bond to the targeting fragment (L).

In a further preferred embodiment said X² is

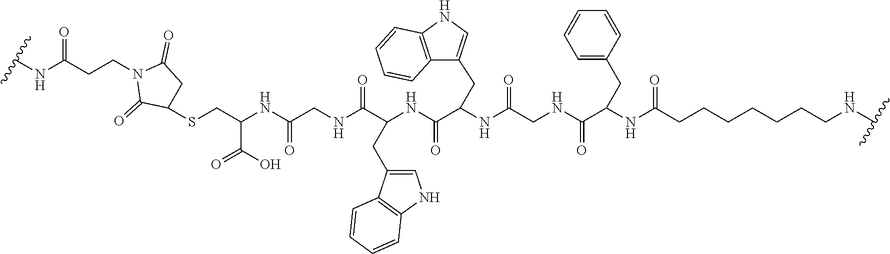 [SMILES: *NCCCCCCCC(=O)NC(Cc1ccccc1)C(=O)NCC(=O)NC(Cc1c[nH]c2ccccc12)C(=O)NC(Cc1c[nH]c2ccccc12)C(=O)NCC(=O)NC(CSC1CC(=O)N(CCC(=O)N*)C1=O)C(=O)O]

and said L of said triconjugate is the DUPA residue (HOOC(CH₂)₂—CH(COOH)—NH—CO—NH—CH(COOH)—(CH₂)₂—CO—). Preferably the wavy line on the left side is a bond to the PEG fragment (—[OCH₂—CH₂]ₘ—) and the wavy line on the right side is a bond to the DUPA residue.

In a further preferred embodiment said X² is

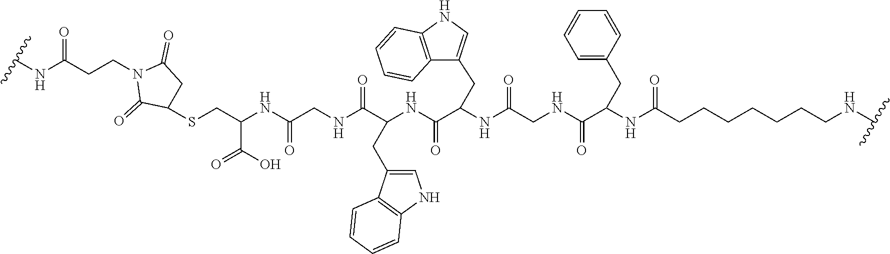 [SMILES: *NCCCCCCCC(=O)NC(Cc1ccccc1)C(=O)NCC(=O)NC(Cc1c[nH]c2ccccc12)C(=O)NC(Cc1c[nH]c2ccccc12)C(=O)NCC(=O)NC(CSC1CC(=O)N(CCC(=O)N*)C1=O)C(=O)O]

and said L of said triconjugate is the DUPA residue (HOOC(CH₂)₂—CH(COOH)—NH—CO—NH—CH(COOH)—(CH₂)₂—CO—), wherein the terminus with the amide group of said X² is bonded to the PEG fragment (—[OCH₂—CH₂]ₘ—) and wherein the terminus with the amine functionality is bonded to the DUPA residue (HOOC(CH₂)₂—CH(COOH)—NH—CO—NH—CH(COOH)—(CH₂)₂—CO—).

In some embodiments, X² is selected from:

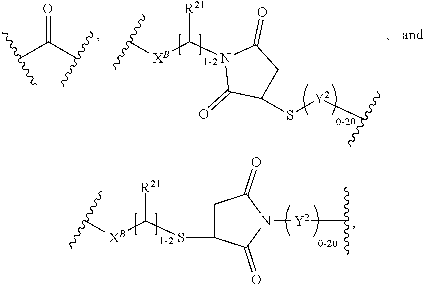

wherein X^{B} is —C(O)NH— or —NH—C(O)—, and wherein Y² and R²¹ are as defined above. Preferably the wavy line on the left side is a bond to the PEG fragment (—[OCH₂—CH₂]ₘ—) and the wavy line on the right side is a bond to the targeting fragment (L).

In some embodiments, X² is selected from:

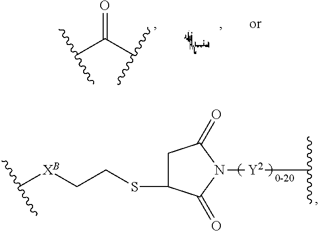

wherein X^{B} is —C(O)NH— or —NH—C(O)—, and wherein Y² and R²¹ are as defined above. Preferably the wavy line on the left side is a bond to the PEG fragment (—[OCH₂—CH₂]ₘ—) and the wavy line on the right side is a bond to the targeting fragment (L).

In some embodiments, X² is selected from:

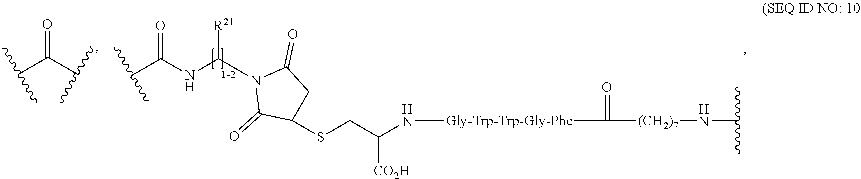

wherein SEQ ID NO:10 is defined as W1-Gly-Trp-Trp-Gly-Phe-W2,
wherein W1 is

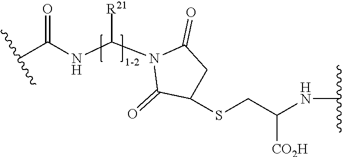

### and W2 is

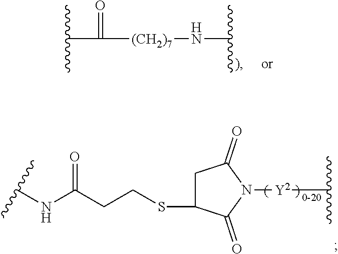

wherein Y² and R²¹ are as defined above. Preferably the wavy line on the left side is a bond to the PEG fragment (—[OCH₂—CH₂]ₘ—) and the wavy line on the right side is a bond to the targeting fragment (L).

In some embodiments, X² is selected from:

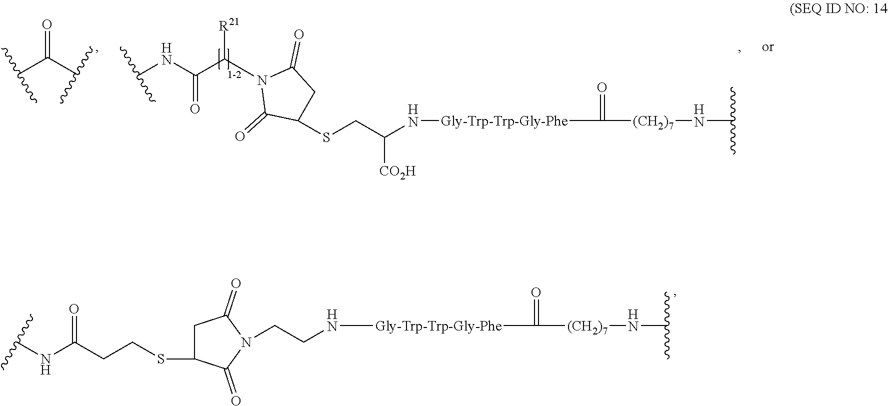

wherein SEQ ID NO:14 is defined as W9-Gly-Trp-Trp-Gly-Phe-W10, wherein W9 is

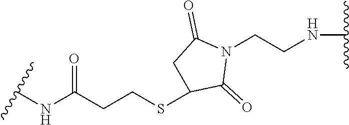 [SMILES: *NCCN1C(=O)CC(SCCC(=O)N*)C1=O]

### and W10 is

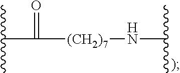

wherein R²¹ is as defiend above; preferably R²¹ is —H or —CH₂—NH₂; more preferably —H. Preferably the wavy line on the left side is a bond to the PEG fragment (—[OCH₂—CH₂]ₘ—) and the wavy line on the right side is a bond to the targeting fragment (L).

In some embodiments, X² is selected from:

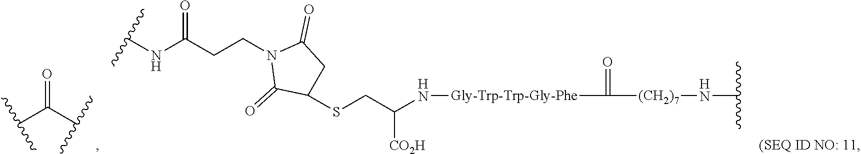

wherein SEQ ID NO:11 is defined as W3-Gly-Trp-Trp-Gly-Phe-W4,
wherein W3 is

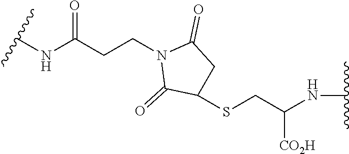 [SMILES: *NC(=O)CCN1C(=O)CC(SCC(N*)C(=O)O)C1=O]

### and W4 is

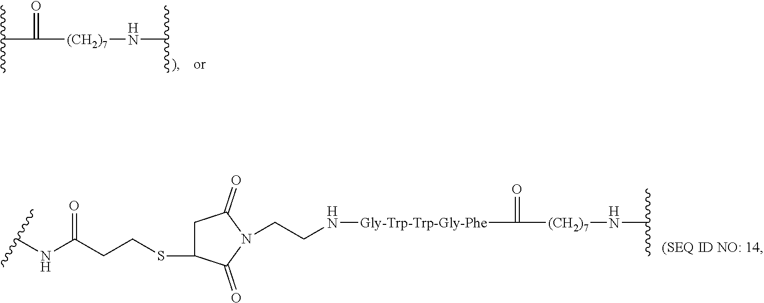

wherein SEQ ID NO:14 is defined as W9-Gly-Trp-Trp-Gly-Phe-W10, wherein W9 is

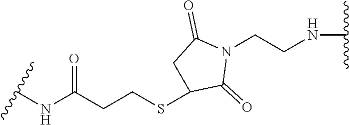 [SMILES: *NCCN1C(=O)CC(SCCC(=O)N*)C1=O]

### and W10 is

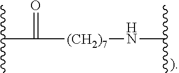

Preferably the wavy line on the left side is a bond to the PEG fragment (—[OCH₂—CH₂]ₘ—) and the wavy line on the right side is a bond to the targeting fragment (L).

In some embodiments, X² is selected from:

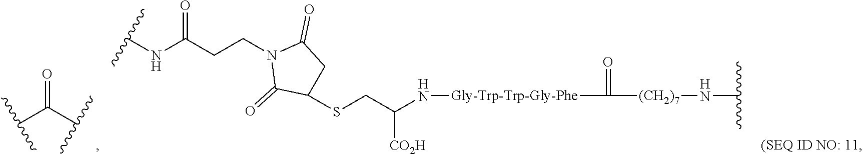

wherein SEQ ID NO:11 is defined as W3-Gly-Trp-Trp-Gly-Phe-W4, wherein W3 is

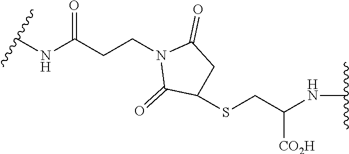 [SMILES: *NC(=O)CCN1C(=O)CC(SCC(N*)C(=O)O)C1=O]

### and W4 is

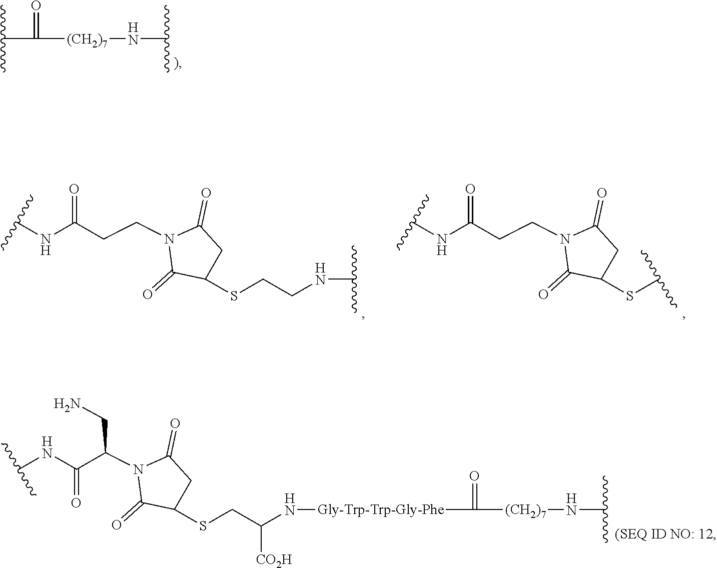

wherein SEQ ID NO: 12 is defined as W5-Gly-Trp-Trp-Gly-Phe-W6, wherein W5 is

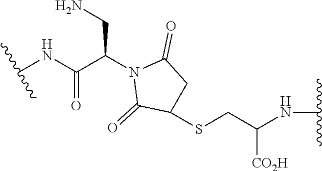 [SMILES: *NC(=O)[C@@H](CN)N1C(=O)CC(SCC(N*)C(=O)O)C1=O]

### and W6 is

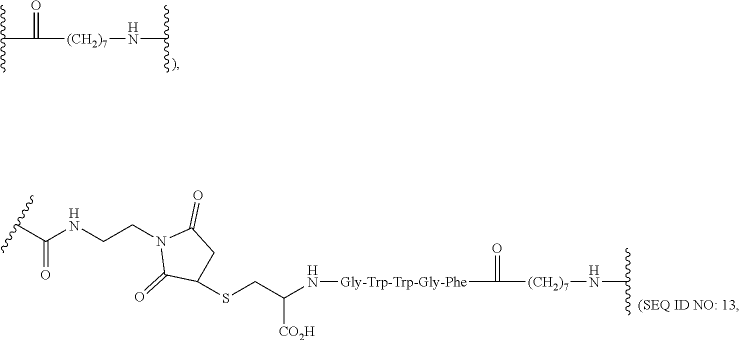

wherein SEQ ID NO:13 is defined as W7-Gly-Trp-Trp-Gly-Phe-W8, wherein W7 is

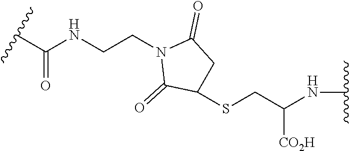 [SMILES: *NC(CSC1CC(=O)N(CCNC(*)=O)C1=O)C(=O)O]

### and W8 is

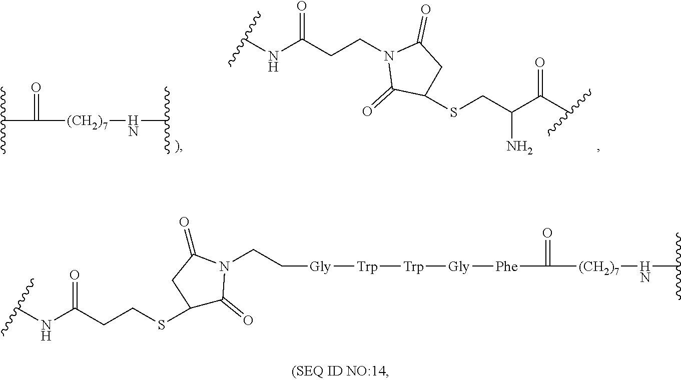

wherein SEQ ID NO:14 is defined as W9-Gly-Trp-Trp-Gly-Phe-W10, wherein W9 is

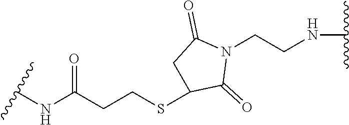 [SMILES: *NCCN1C(=O)CC(SCCC(=O)N*)C1=O]

### and W10 is

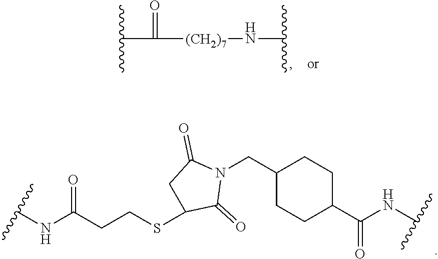

Preferably the wavy line on the left side is a bond to the PEG fragment (—[OCH₂—CH₂]ₘ—) and the wavy line on the right side is a bond to the targeting fragment (L).

In some embodiments, X² is:

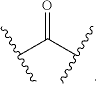 [SMILES: *C(*)=O]

In some embodiments, X² is:

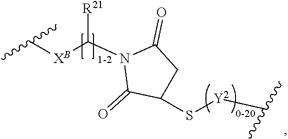

wherein X^{B} is —C(O)NH— or —NH—C(O)—. Preferably the wavy line on the left side is a bond to the PEG fragment (—[OCH₂—CH₂]ₘ—) and the wavy line on the right side is a bond to the targeting fragment (L).

In some embodiments, X² is:

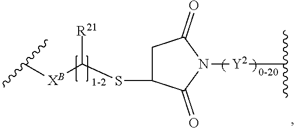

wherein X^{B} is —C(O)NH— or —NH—C(O)—. Preferably the wavy line on the left side is a bond to the PEG fragment (—[OCH₂—CH₂]ₘ—) and the wavy line on the right side is a bond to the targeting fragment (L).

In some embodiments, the composition comprises a conjugate of the Formula IA:

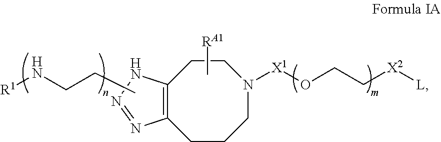

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-1:

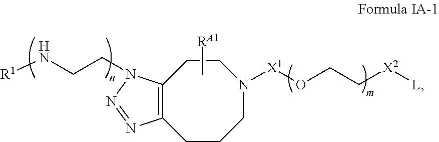

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-2:

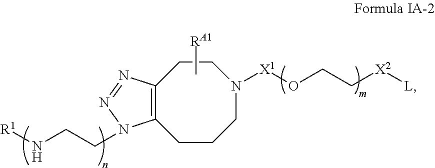

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-3:

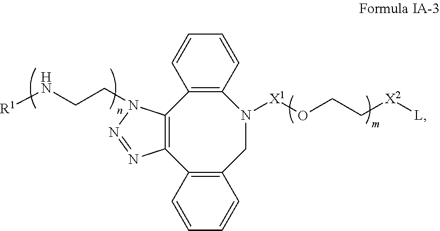

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, preferably and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-3a:

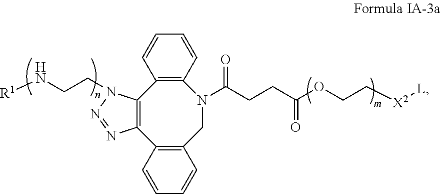

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, preferably and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-3b:

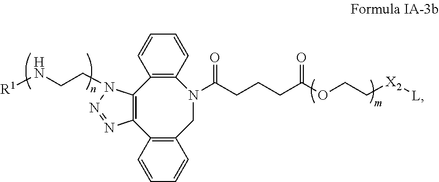

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-3c:

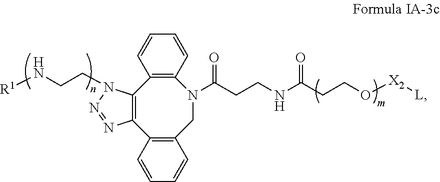

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-3d:

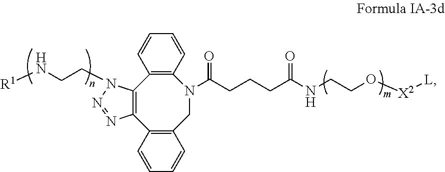

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-4:

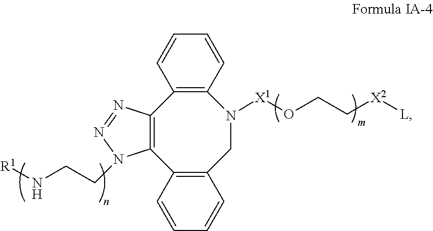

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-4a:

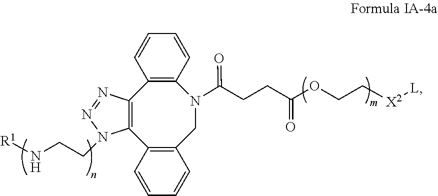

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-4b:

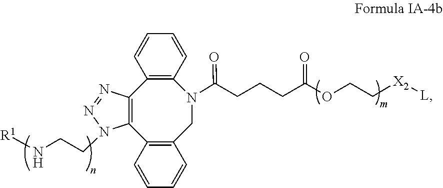

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-4c:

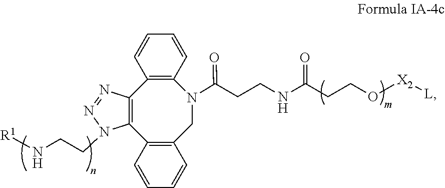

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-4d:

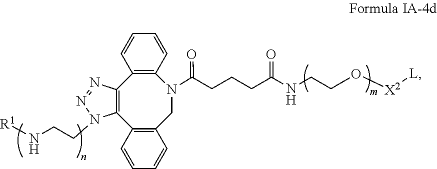

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-5:

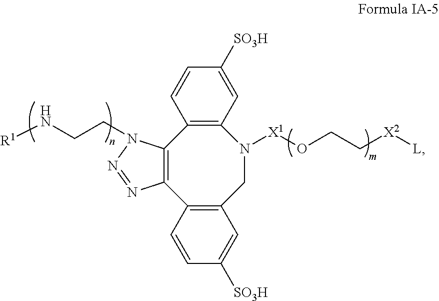

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-6:

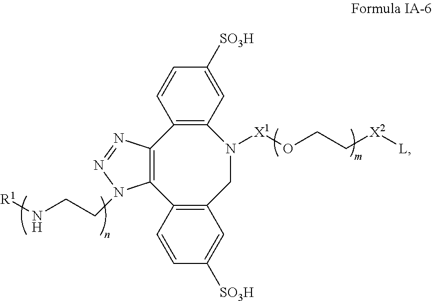

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-7:

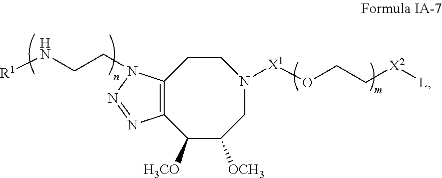

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4

In some embodiments, the composition comprises a conjugate of the Formula IA-7a:

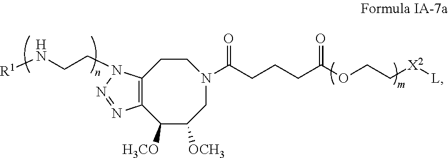

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-8:

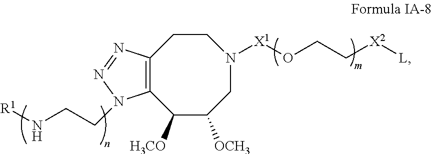

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 35, and again further preferably 25 to 60, preferably 36.

In some embodiments, the composition comprises a conjugate of the Formula IA-8a:

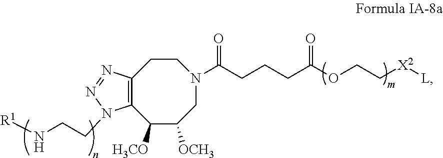

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-9:

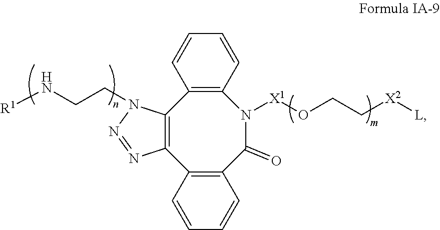

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 35, and again further preferably 25 to 60, preferably 36.

In some embodiments, the composition comprises a conjugate of the Formula IA-9a:

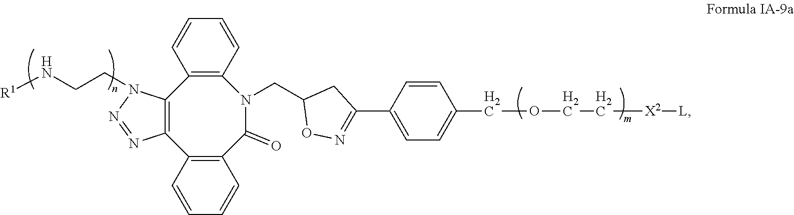

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IA-10:

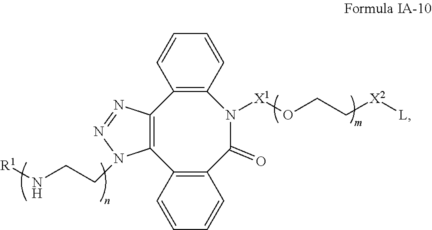

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 35, and again further preferably 25 to 60, preferably 36.

In some embodiments, the composition comprises a conjugate of the Formula IA-10a:

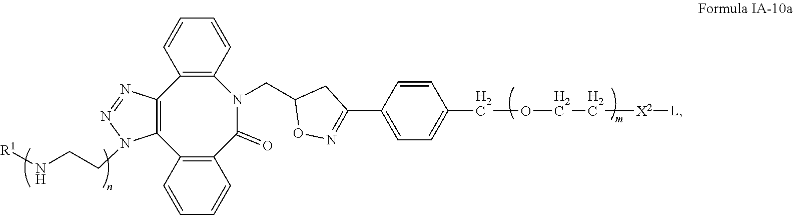

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IB:

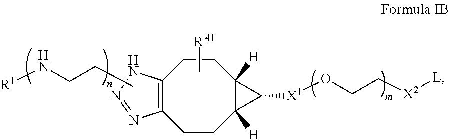

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 35, and again further preferably 25 to 60, preferably 36.

In some embodiments, the composition comprises a conjugate of the Formula IB-1:

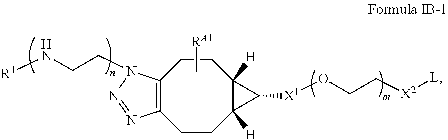

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IB-1a:

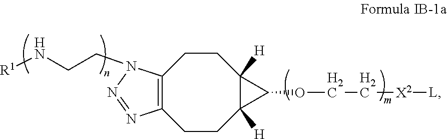

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IB-2:

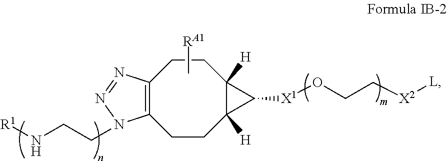

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IB-2a:

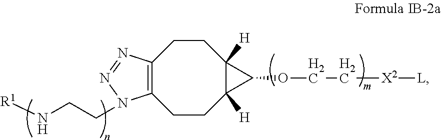

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IC:

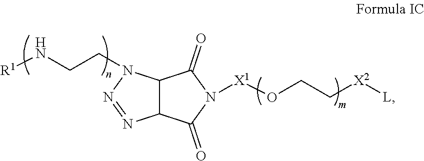

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IC-1:

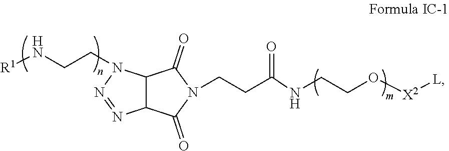

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID:

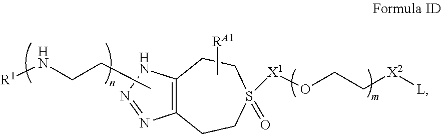

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-1:

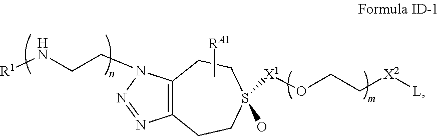

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-1a:

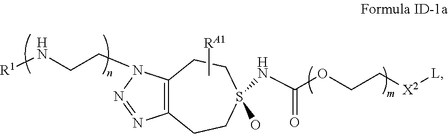

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-2:

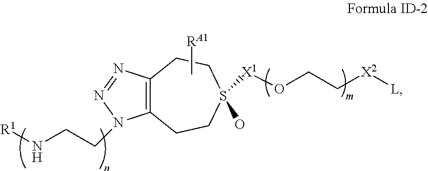

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-2a:

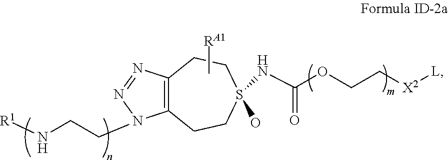

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-3:

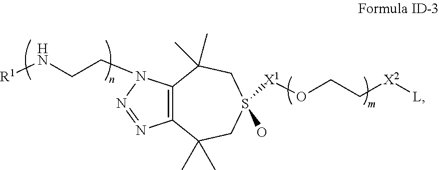

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-3a:

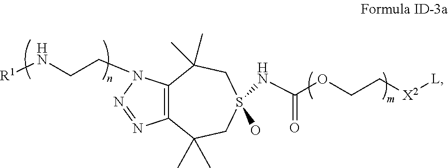

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-4:

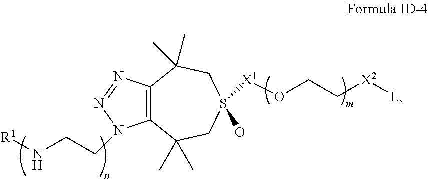

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula ID-4a:

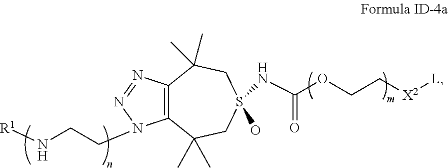

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE:

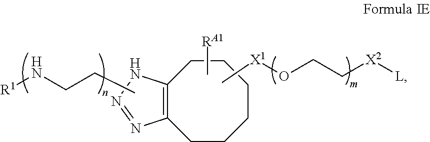

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-1:

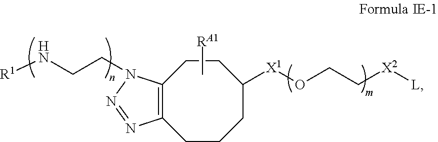

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-2:

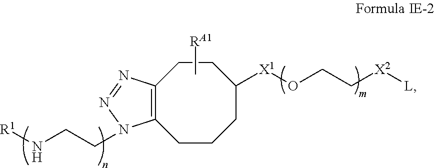

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-3:

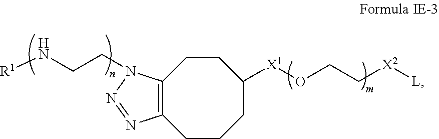

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-3a:

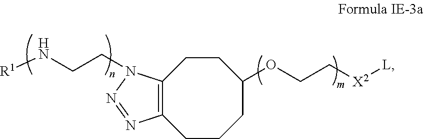

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-4:

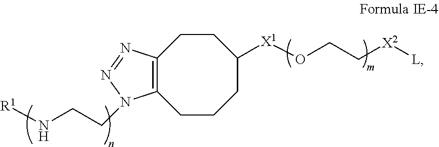

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-4a:

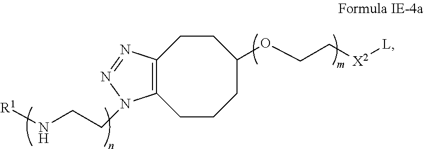

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-5:

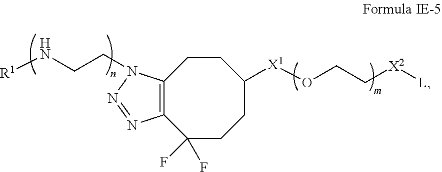

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 35, and again further preferably 25 to 60, preferably 36.

In some embodiments, the composition comprises a conjugate of the Formula IE-5a:

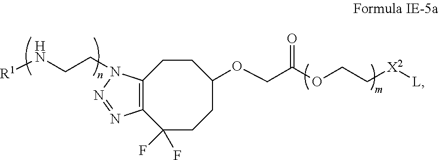

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-6:

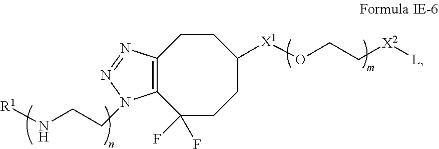

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-6a:

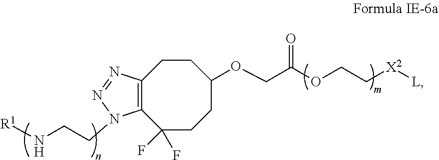

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-7:

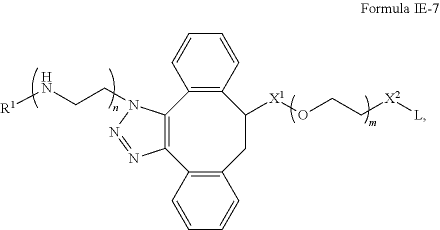

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 35, and again further preferably 25 to 60, preferably 36.

In some embodiments, the composition comprises a conjugate of the Formula IE-7a:

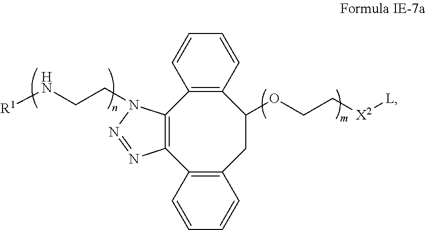

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-8:

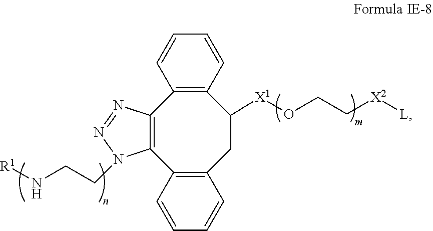

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-8a:

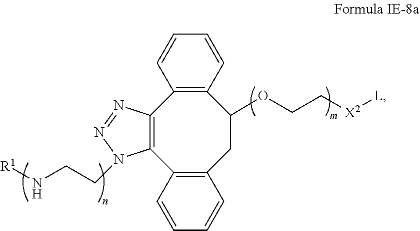

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-9:

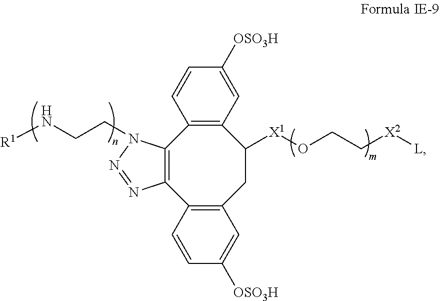

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-9a:

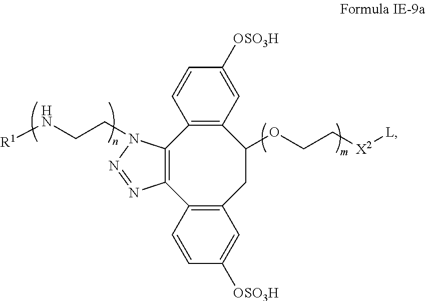

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-10:

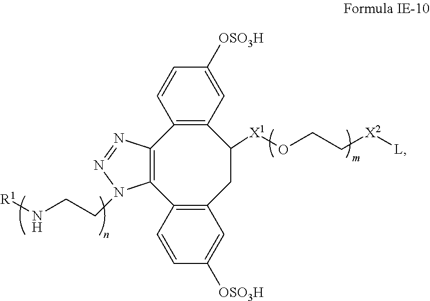

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)—units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-10a:

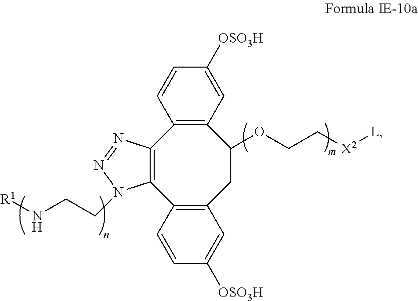

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-11:

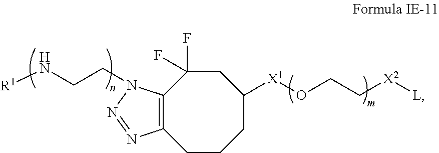

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-11a:

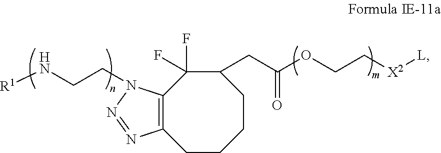

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-11b:

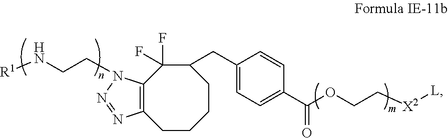

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-12:

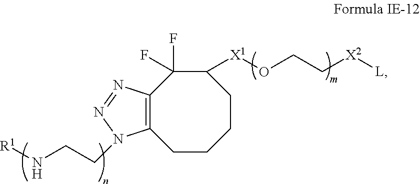

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-12a:

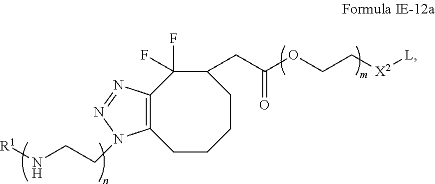

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-12b:

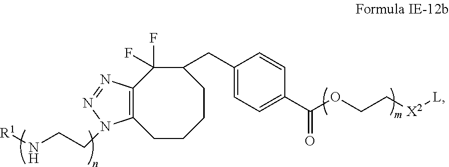

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-13:

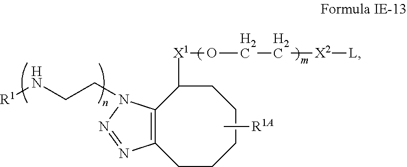

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-13a:

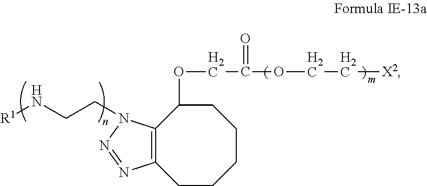

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-13b:

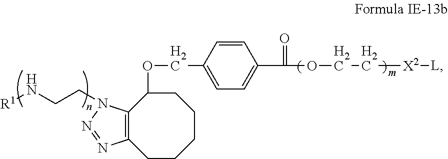

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-13c:

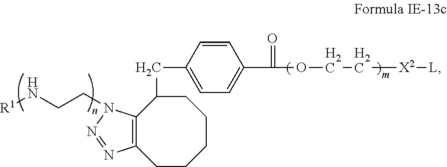

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-13d:

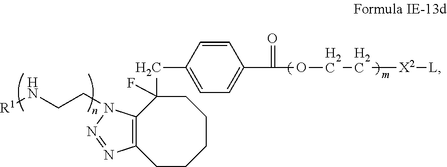

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-14:

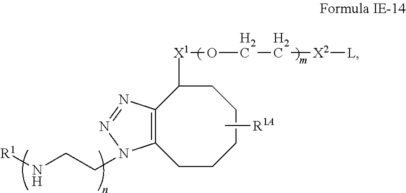

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-14a:

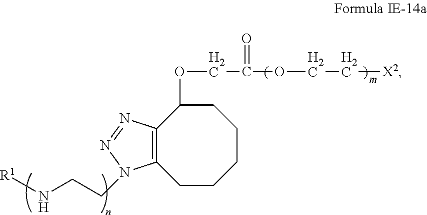

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-14b:

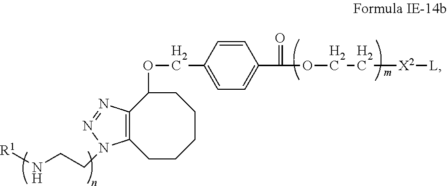

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-14c:

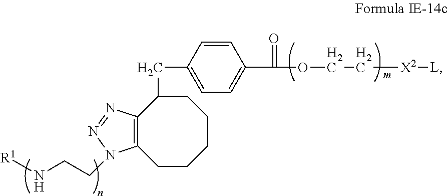

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IE-14d:

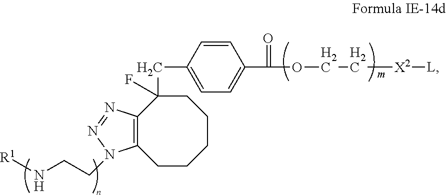

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IH:

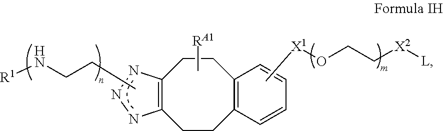

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IH′:

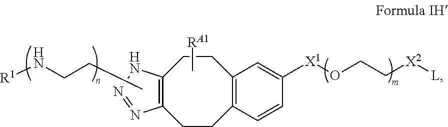

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IH-1:

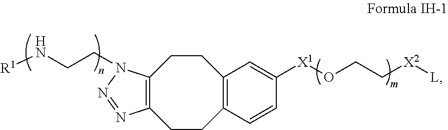

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IH-1a:

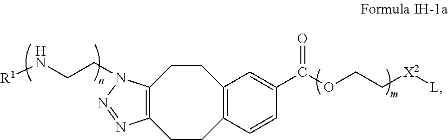

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IH-2:

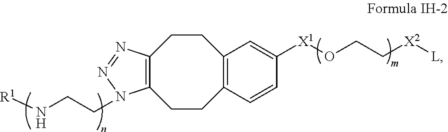

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IH-2a:

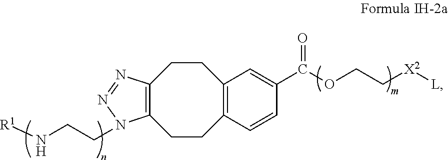

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IJ:

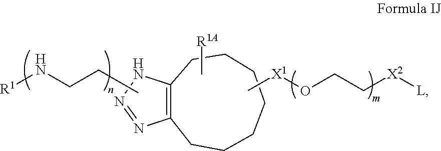

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IJ-1:

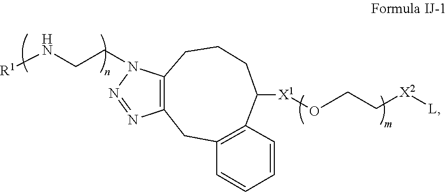

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IJ-1a:

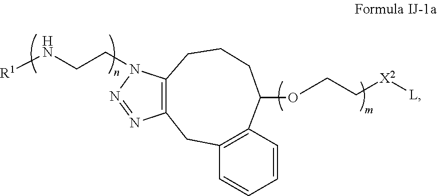

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IJ-2:

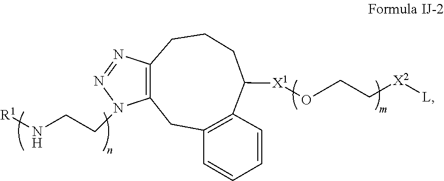

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IJ-2a:

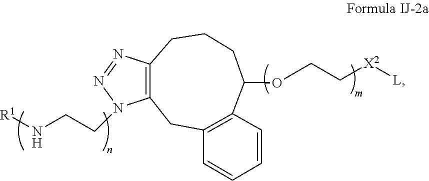

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IJ-3:

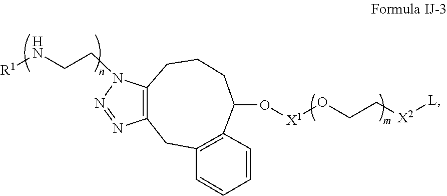

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IJ-4:

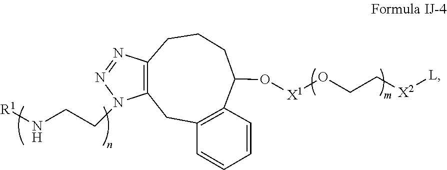

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IK:

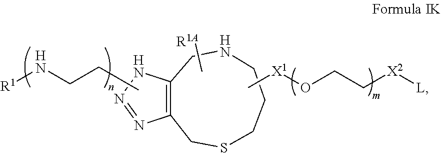

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IK-1:

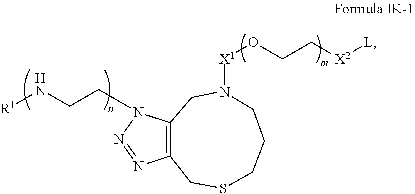

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IK-2:

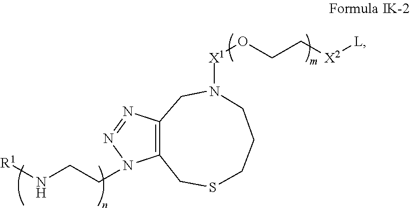

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IK-3:

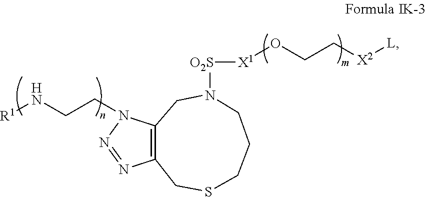

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IK-4:

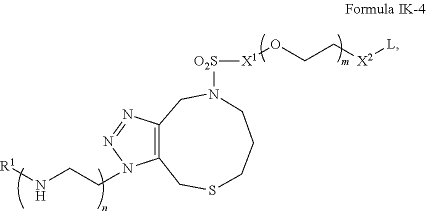

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IK-3a:

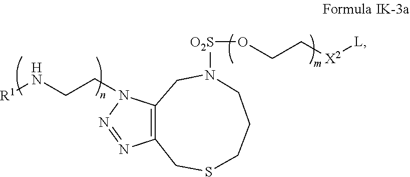

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IK-4a:

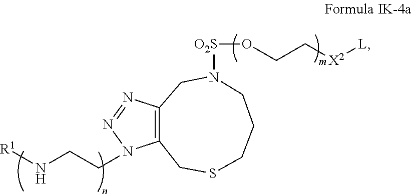

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IL:

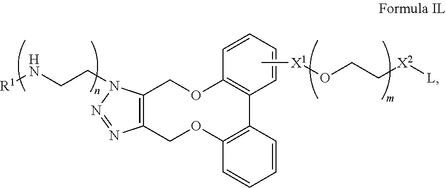

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IM:

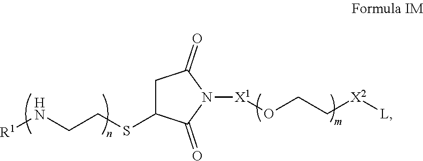

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IN:

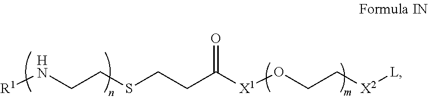

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IO:

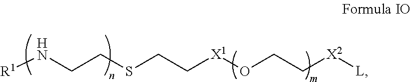

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IP:

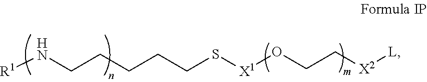

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IQ:

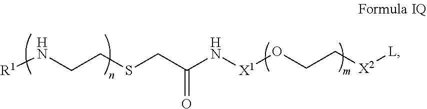

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IR:

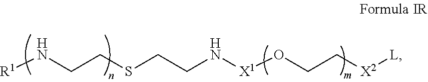

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete 5 number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, the composition comprises a conjugate of the Formula IQ:

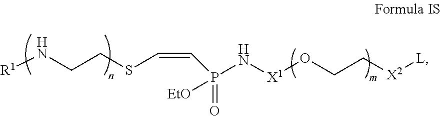

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In a further preferred embodiment, said conjugate of Formula I is selected from:

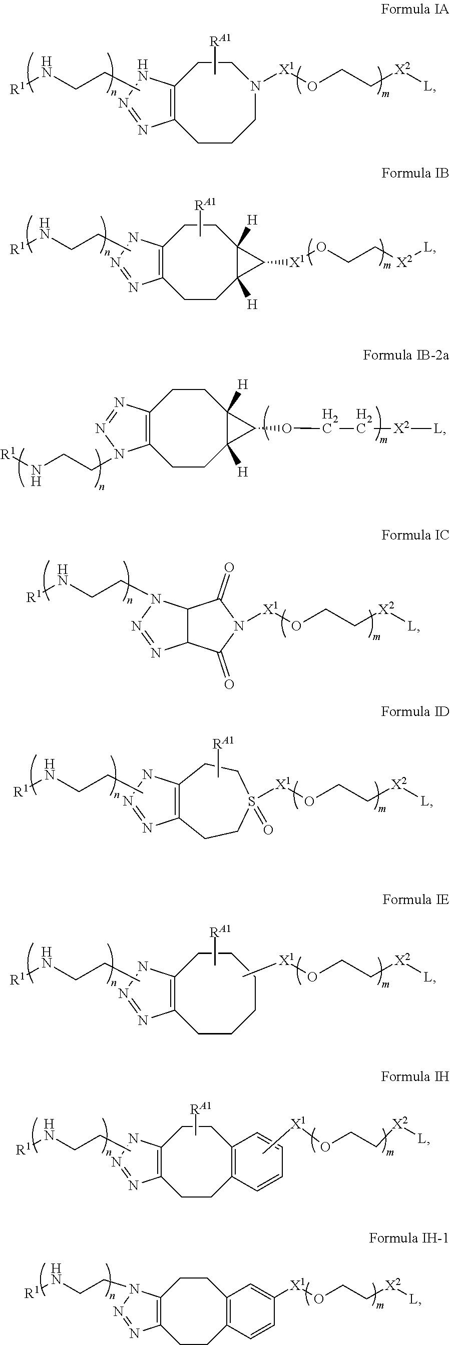

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, said conjugate of Formula I is selected from:

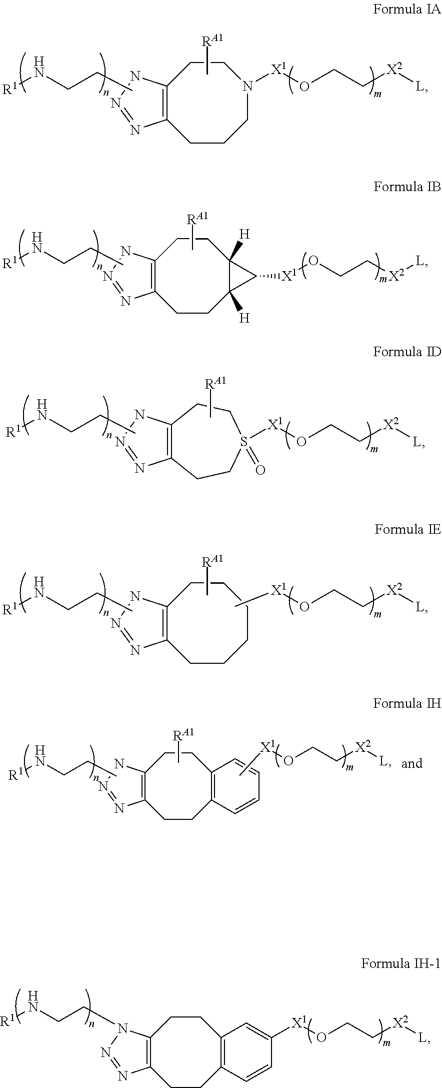

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In preferred embodiments, of any of Formulae IA, IB, IC, ID, IE, and/or IH, R^{A1} is —H.

In another referred embodiment, said conjugate of Formula I is selected from:

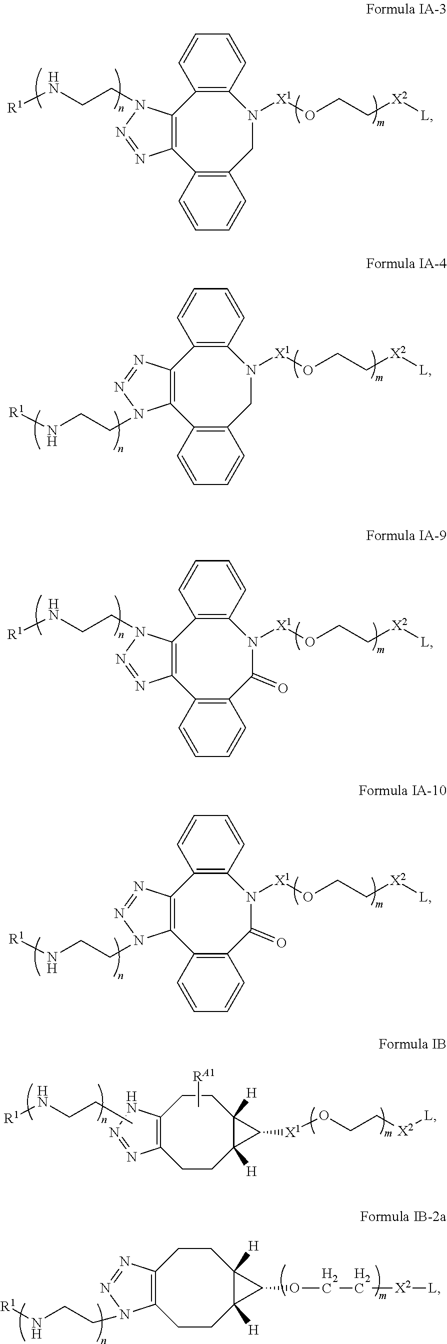

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, said conjugate of Formula I is selected from:

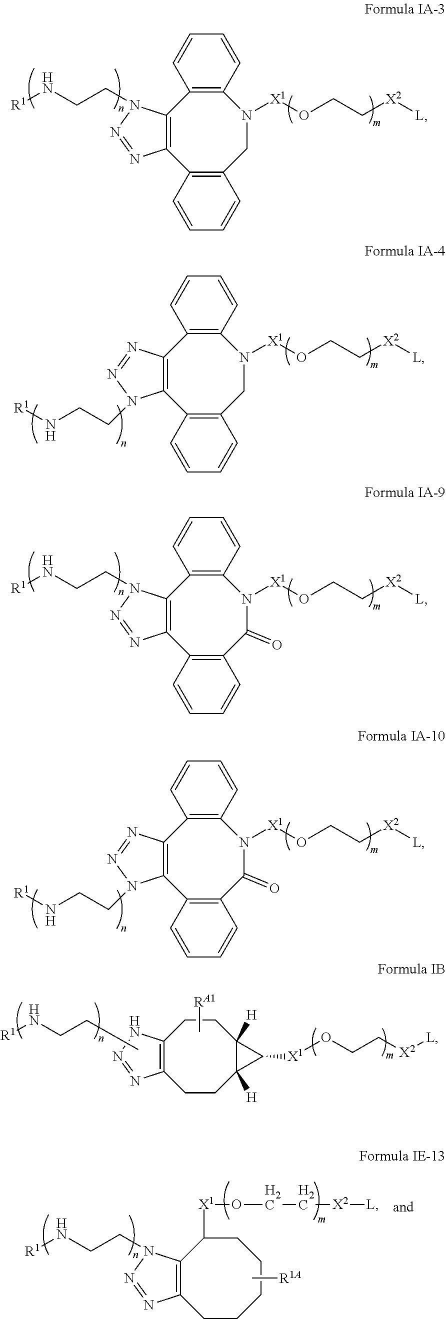

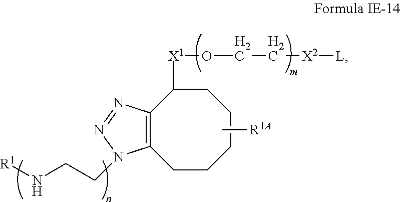

preferably wherein n is between about 280 and about 700 with a dispersity of about 3 or less, more preferably between about 350 and about 630 with a dispersity of about 2 or less, and again more preferably between about 400 and 580 with a dispersity about 1.2 or less, and preferably wherein m is between about 2 and about 80 and a dispersity of about 2 or less, more preferably between about 2 and about 70 with a dispersity of about 1.8 or less; again more preferably between about 2 and about 50 repeating units with a dispersity of about 1.5, or alternatively m is a discrete number of repeating units, and preferably wherein m is a discrete number of repeating —(O—CH₂—CH₂)— units, wherein said discrete number m is any integer of 4 to 100, preferably of 4 to 60, and further preferably 12, 24 or 36, and again further preferably 25 to 60, preferably 36.

In some embodiments, said conjugate of Formula I is selected from:

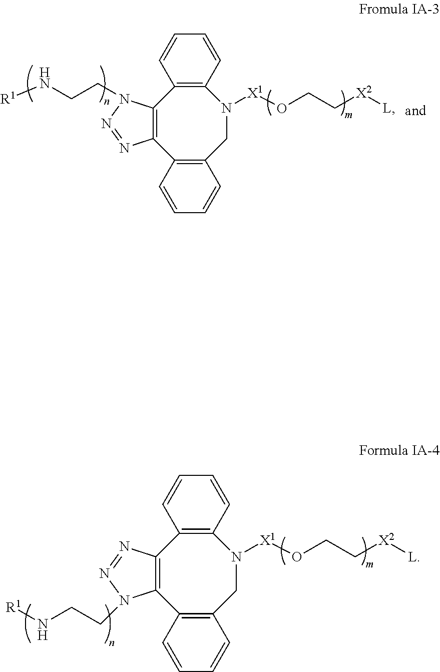

In some embodiments, said conjugate of Formula I is selected from:

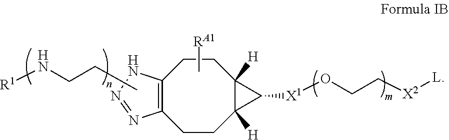

In some embodiments, said conjugate of Formula I is selected from:

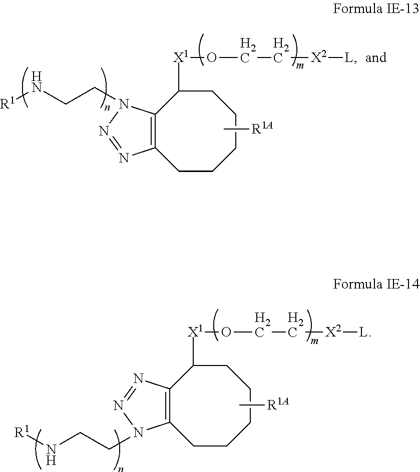

In some embodiments, the composition comprises a conjugate of the formula:

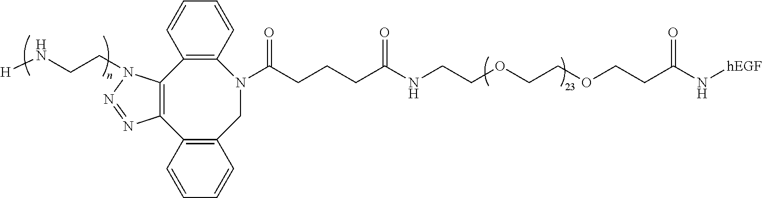

preferably wherein n is between about 400 and 580 with a dispersity about 1.2 or less.

In some embodiments, the composition comprises a conjugate of the formula:

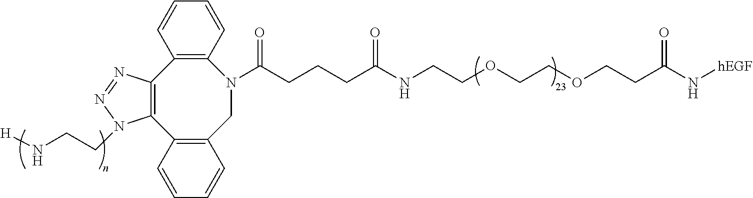

preferably wherein n is between about 400 and 580 with a dispersity about 1.2 or less.

In some embodiments, the composition comprises a conjugate of the formula:

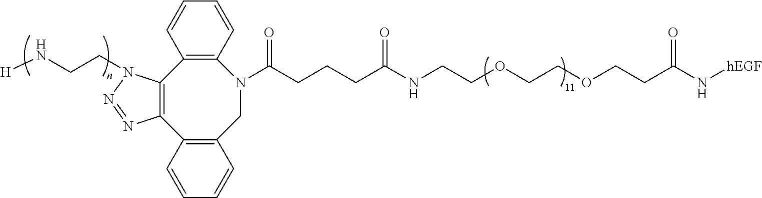

preferably wherein n is between about 400 and 580 with a dispersity about 1.2 or less.

In some embodiments, the composition comprises a conjugate of the formula:

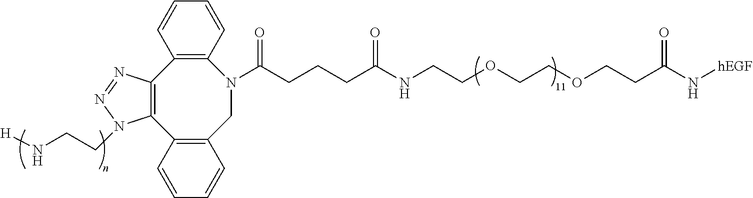

preferably wherein n is between about 400 and 580 with a dispersity about 1.2 or less.

In some embodiments, the composition comprises a conjugate of the formula:

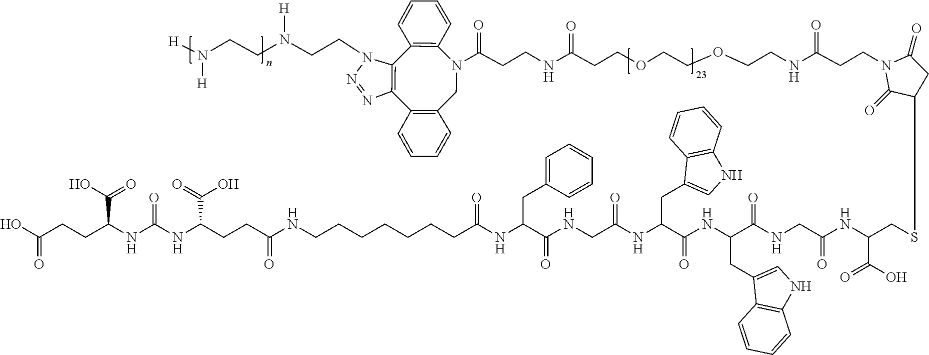

preferably wherein n is between about 400 and 580 with a dispersity about 1.2 or less.

In some embodiments, the composition comprises a conjugate of the formula:

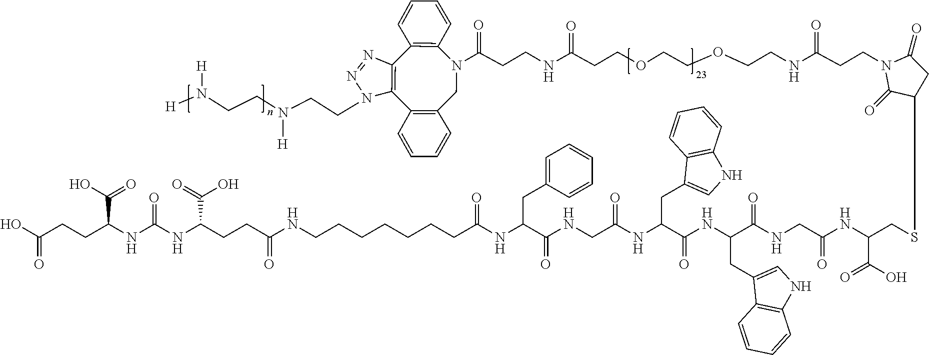

preferably wherein n is between about 400 and 580 with a dispersity about 1.2 or less.

In some embodiments, the composition comprises a conjugate of the formula:

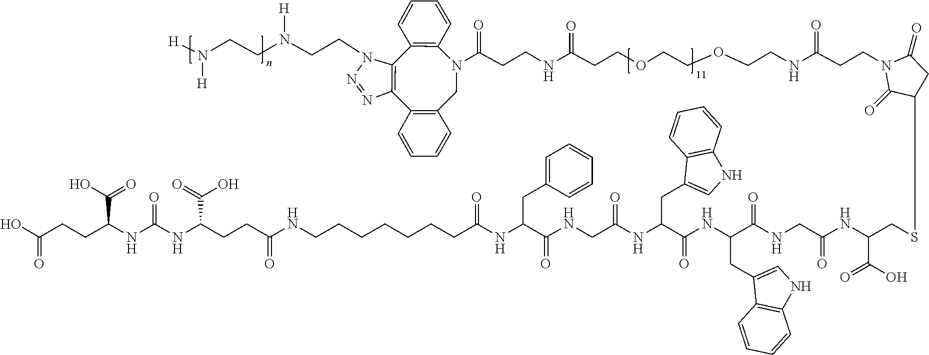

preferably wherein n is between about 400 and 580 with a dispersity about 1.2 or less.

In some embodiments, the composition comprises a conjugate of the formula:

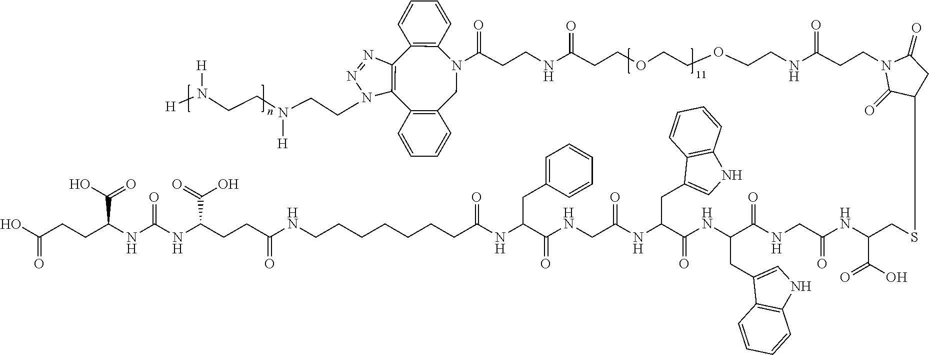

preferably wherein n is between about 400 and 580 with a dispersity about 1.2 or less.

In some embodiments, the composition comprises a conjugate of the formula:

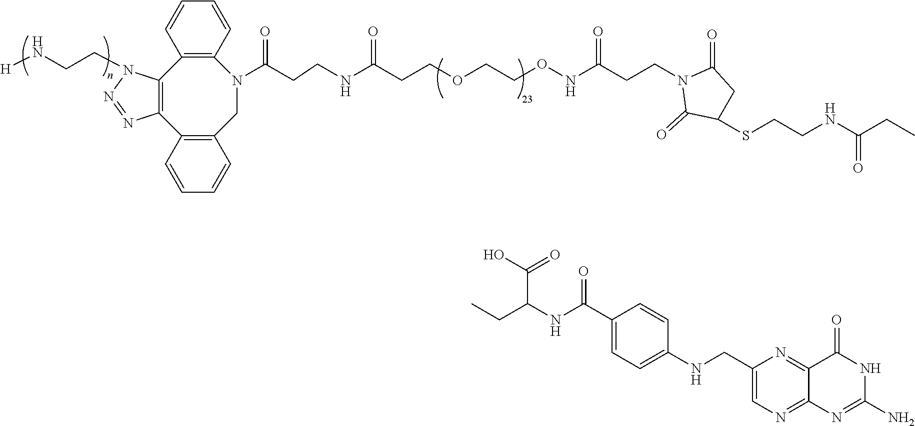

preferably wherein n is between about 400 and 580 with a dispersity about 1.2 or less.

In some embodiments, the composition comprises a conjugate of the formula:

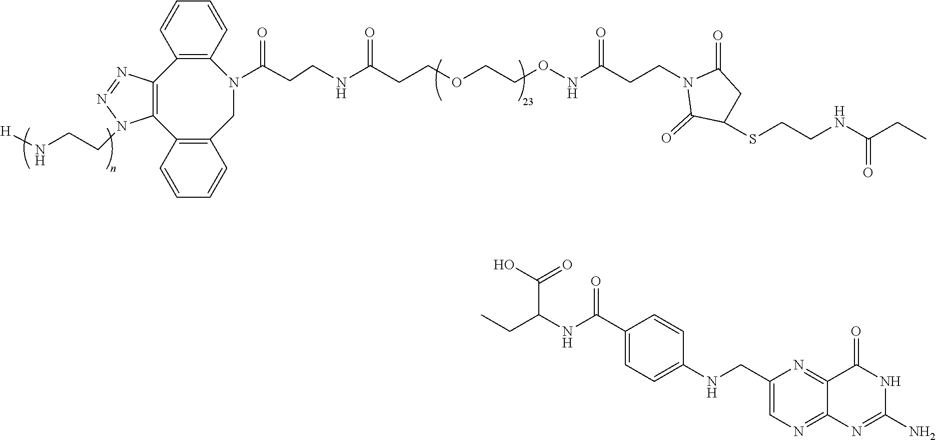

preferably wherein n is between about 400 and 580 with a dispersity about 1.2 or less.

In some embodiments, the composition comprises a conjugate of the formula:

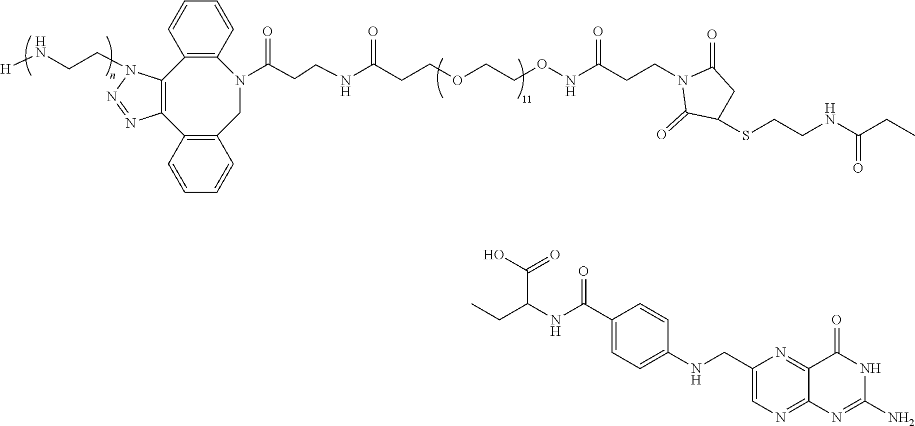

preferably wherein n is between about 400 and 580 with a dispersity about 1.2 or less

In some embodiments, the composition comprises a conjugate of the formula:

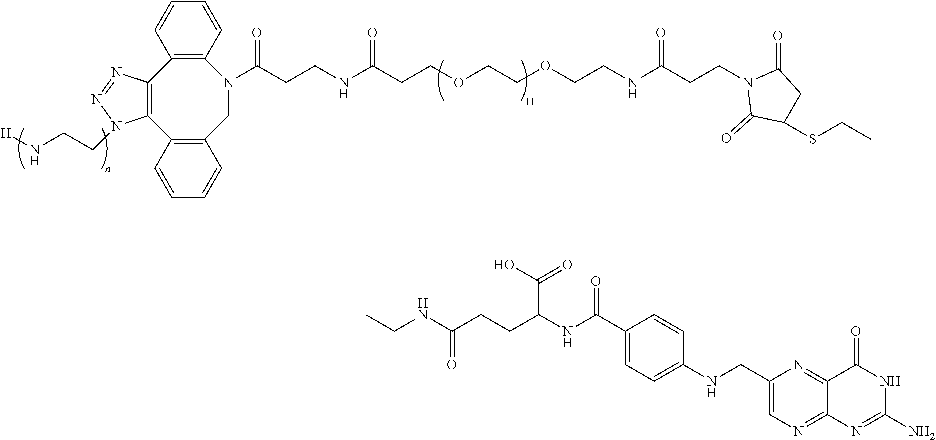

preferably wherein n is between about 400 and 580 with a dispersity about 1.2 or less.

In a preferred embodiment, the composition comprises a conjugate comprising Compound 1a, Compound 1b, Compound 4a, Compound 4b, Compound 7a, Compound 7b, Compound 10a, Compound 10b, Compound 14, Compound 17a, Compound 17b, Compound 18, Compound 19, Compound 22a, Compound 22b, Compound 28a, Compound 28b, Compound 31a, Compound 31b, Compound 38a, Compound 38b, Compound 43, Compound 47a, Compound 47b, Compound 51a, Compound 51b, Compound 56a, Compound 56b, Compound 62a, Compound 62b, Compound 70a, Compound 70b, Compound 72a, Compound 72b, Compound 75a, Compound 75b, Compound 78a, Compound 78b, Compound 81, Compound 82a Compound 82b and/or Compound 83.

In a preferred embodiment, the composition comprises a conjugate selected from Compound 1a, Compound 1b, Compound 4a, Compound 4b, Compound 7a, Compound 7b, Compound 10a, Compound 10b, Compound 14, Compound 17a, Compound 17b, Compound 18, Compound 19, Compound 22a, Compound 22b, Compound 28a, Compound 28b, Compound 31a, Compound 31b, Compound 38a, Compound 38b, Compound 43, Compound 47a, Compound 47b, Compound 51a, Compound 51b, Compound 56a, Compound 56b, Compound 62a, Compound 62b, Compound 70a, Compound 70b, Compound 72a, Compound 72b, Compound 75a, Compound 75b, Compound 78a, Compound 78b, Compound 81, Compound 82a Compound 82b and/or Compound 83.

In a preferred embodiment, the composition comprises a conjugate comprising Compound 1a, and/or Compound 1b. In some embodiments, the composition comprises a conjugate comprising Compound 4a and/or Compound 4b. In some embodiments, the composition comprises a conjugate comprising Compound 7a and/or Compound 7b. In some embodiments, the composition comprises a conjugate comprising Compound 10a and/or Compound 10b. In some embodiments, the composition comprises a conjugate comprising Compound 14. In some embodiments, the composition comprises a conjugate comprising Compound 17a and/or Compound 17b. In some embodiments, the composition comprises a conjugate comprising Compound 18. In some embodiments, the composition comprises a conjugate comprising Compound 19. In some embodiments, the composition comprises a conjugate comprising Compound 22a and/or Compound 22b. In some embodiments, the composition comprises a conjugate comprising Compound 28a and/or Compound 28b. In some embodiments, the composition comprises a conjugate comprising Compound 31a and/or Compound 31b. In some embodiments, the composition comprises a conjugate comprising Compound 38a and/or Compound 38b. In some embodiments, the composition comprises a conjugate comprising Compound 43. In some embodiments, the composition comprises a conjugate comprising Compound 47a and/or Compound 47b. In some embodiments, the composition comprises a conjugate comprising Compound 51a and/or Compound 51b. In some embodiments, the composition comprises a conjugate comprising Compound 56a and/or Compound 56b. In some embodiments, the composition comprises a conjugate comprising Compound 62a and/or Compound 62b. In some embodiments, the composition comprises a conjugate comprising Compound 70a and/or Compound 70b. In some embodiments, the composition comprises a conjugate comprising Compound 72a and/or Compound 72b. In some embodiments, the composition comprises a conjugate comprising Compound 75a and/or Compound 75b. In some embodiments, the composition comprises a conjugate comprising Compound 78a and/or Compound 78b. In some embodiments, the composition comprises a conjugate comprising Compound 81. In some embodiments, the composition comprises a conjugate comprising Compound 82a and/or Compound 82b. In some embodiments, the composition comprises a conjugate comprising Compound 83.

In a preferred embodiment, the composition comprises a conjugate, wherein said conjugate is Compound 1a, and/or Compound 1b. In a preferred embodiment, the composition comprises a conjugate, wherein said conjugate is Compound 4a and/or Compound 4b. In a preferred embodiment, the composition comprises a conjugate, wherein said conjugate is Compound 7a and/or Compound 7b. In a preferred embodiment, the composition comprises a conjugate, wherein said conjugate is Compound 10a and/or Compound 10b. In a preferred embodiment, the composition comprises a conjugate, wherein said conjugate is Compound 14. In a preferred embodiment, the composition comprises a conjugate, wherein said conjugate is Compound 17a and/or Compound 17b. In a preferred embodiment, the composition comprises a conjugate, wherein said conjugate is Compound 18. In a preferred embodiment, the composition comprises a conjugate, wherein said conjugate is Compound 19. In a preferred embodiment, the composition comprises a conjugate, wherein said conjugate is Compound 22a and/or Compound 22b. In a preferred embodiment, the composition comprises a conjugate, wherein said conjugate is Compound 28a and/or Compound 28b. In a preferred embodiment, the composition comprises a conjugate, wherein said conjugate is Compound 31a and/or Compound 31b. In a preferred embodiment, the composition comprises a conjugate, wherein said conjugate is Compound 38a and/or Compound 38b. In a preferred embodiment, the composition comprises a conjugate, wherein said conjugate is Compound 43. In a preferred embodiment, the composition comprises a conjugate, wherein said conjugate is Compound 47a and/or Compound 47b. In a preferred embodiment, the composition comprises a conjugate, wherein said conjugate is Compound 51a and/or Compound 51b. In a preferred embodiment, the composition comprises a conjugate, wherein said conjugate is Compound 56a and/or Compound 56b. In a preferred embodiment, the composition comprises a conjugate, wherein said conjugate is Compound 62a and/or Compound 62b. In a preferred embodiment, the composition comprises a conjugate, wherein said conjugate is Compound 70a and/or Compound 70b. In a preferred embodiment, the composition comprises a conjugate, wherein said conjugate is Compound 72a and/or Compound 72b. In a preferred embodiment, the composition comprises a conjugate, wherein said conjugate is Compound 75a and/or Compound 75b. In a preferred embodiment, the composition comprises a conjugate, wherein said conjugate is Compound 78a and/or Compound 78b. In a preferred embodiment, the composition comprises a conjugate, wherein said conjugate is Compound 81. In a preferred embodiment, the composition comprises a conjugate, wherein said conjugate is Compound 82a and/or Compound 82b. In a preferred embodiment, the composition comprises a conjugate, wherein said conjugate is Compound 83.

### Polyplexes

The inventive compositions comprise a nucleic acid, wherein said nucleic acid and said conjugate form a polyplex. In a preferred embodiment, said nucleic acid is non-covalently bound to said conjugate. This facilitates the dissociation of the nucleic acid from the targeting fragment following arrival to the targeted cell or tissue and its internalization in the targeted cell or tissue, preferably tumor cell or tumor tissue causing the production of, for example, chemokines, as shown herein. The production of chemokines will attract immune cells to the tumor site.

The inventive polyplex provides efficient delivery of the nucleic acid into cells harboring the target cell surface receptor. As described herein, the targeting fragment comprised by the inventive polyplex is capable of binding to the target cell surface receptor.

In a preferred embodiment, said nucleic acid is a RNA. In a preferred embodiment, said nucleic acid is a single stranded RNA (ssRNA). In a preferred embodiment, said ssRNA is a messenger RNA (mRNA).

In another aspect, the present invention provides a polyplex comprising a conjugate of Formula I*, preferably of Formula I, or a pharmaceutically acceptable salt, solvate, hydrate, tautomer or enantiomer thereof, and a nucleic acid, wherein said nucleic acid is preferably non-covalently bound to said conjugate

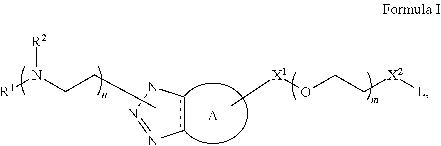

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X², L, m and n.

In another aspect, the present invention provides a polyplex comprising a conjugate of Formula I, or a pharmaceutically acceptable salt, solvate, hydrate, tautomer or enantiomer thereof, and a nucleic acid, wherein said nucleic acid is preferably non-covalently bound to said conjugate:

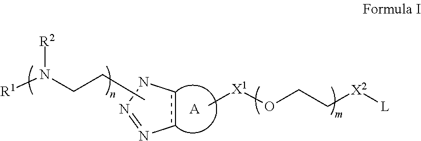

wherein:
[symbol] is a single bond or a double bond;
n is any integer between 1 and 1500, preferably any integer between 2 and 1500;
m is a discrete number of repeating units m of 2 to 100, preferably of a discrete number of repeating units m of 4 to 60;
R¹ is an initiation residue, wherein preferably R¹ is —H or —CH₃;
R² is independently —H or an organic residue, wherein at least 80%, preferably wherein 5 at least 90%, of said R² in said —(NR²—CH₂—CH₂)ₙ— is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor. In a preferred embodiment, said R¹ is —H. In a preferred embodiment, said R¹ is —CH₃. In a preferred embodiment, said nucleic acid is a RNA. In a preferred embodiment, said RNA is a ssRNA. In a preferred embodiment, said RNA is a ssRNA. In a preferred embodiment, said RNA is a mRNA. In a preferred embodiment, said ssRNA is a mRNA. In another preferred embodiment said nucleic acid is a DNA, wherein preferably said DNA is a plasmid DNA.

In another aspect, the present invention provides a polyplex comprising a conjugate of Formula I, or a pharmaceutically acceptable salt, solvate, hydrate, tautomer or enantiomer thereof, and a nucleic acid, wherein said nucleic acid is preferably non-covalently bound to said conjugate:

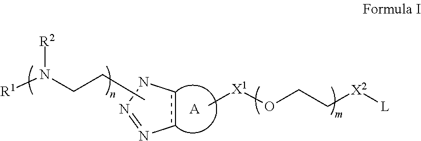

wherein:
[symbol] is a single bond or a double bond;
n is any integer between 1 and 1500, preferably any integer between 2 and 1500;
m is a discrete number of repeating units m of 36;
R¹ is an initiation residue, wherein preferably R¹ is —H or —CH₃;
R² is independently —H or an organic residue, wherein at least 80%, preferably wherein 5 at least 90%, of said R² in said —(NR²—CH₂—CH₂)ₙ— is H; Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor. In a preferred embodiment, said R¹ is —H. In a preferred embodiment, said R¹ is —CH₃. In a preferred embodiment, said nucleic acid is a RNA. In a preferred embodiment, said RNA is a ssRNA. In a preferred embodiment, said RNA is a mRNA. In a preferred embodiment, said ssRNA is a mRNA. In another preferred embodiment said nucleic acid is a DNA, wherein preferably said DNA is a plasmid DNA. In another preferred embodiment said nucleic acid is a DNA, wherein said DNA is a plasmid DNA.

The term “RNA” as used herein relates to a nucleic acid which comprises ribonucleotide residues and preferably being entirely or substantially composed of ribonucleotide residues. “Ribonucleotide” relates to a nucleotide with a hydroxyl group at the 2′-position of a 3-D-ribofuranosyl group. The term “RNA” as used herein comprises double stranded RNA (dsRNA) and single stranded RNA (ssRNA). The term “RNA” further includes isolated RNA such as partially or completely purified RNA, essentially pure RNA, synthetic RNA, recombinantly generated RNA, in vitro transcribed RNA, in vivo transcribed RNA from a template such as a DNA template, and replicon RNA, in particular self-replicating RNA, and includes modified RNA which differs from naturally occurring RNA by addition, deletion, substitution and/or alteration of one or more nucleotides. Such alterations can include addition of non-nucleotide material, such as to the end(s) of an RNA or internally. The RNA may have modified naturally occurring or synthetic ribonucleotides. Nucleotides in RNA can also comprise non-standard nucleotides, such as non-naturally occurring nucleotides or chemically synthesized nucleotides or deoxynucleotides.

The term “single stranded RNA (ssRNA)” generally refers to an RNA molecule to which no complementary nucleic acid molecule (typically no complementary RNA molecule) is associated. ssRNA may contain self-complementary sequences that allow parts of the RNA to fold back and pair with itself to form double helices and secondary structure motifs including without limitation base pairs, stems, stem loops and bulges. The size of the ssRNA strand may vary from 8 nucleotides up to 120000 nucleotides, typically and preferably the size of the ssRNA strand may vary from 8 nucleotides up to 20000 nucleotides.

The term “double stranded RNA (dsRNA)” is RNA with two partially or completely complementary strands. The dsRNA is preferably a fully or partially (interrupted) pair of RNA hybridized together. It can be prepared for example by mixing partially or completely complementary strands ssRNA molecules. It also can be made by mixing defined fully or partially pairing non-homopolymeric or homopolymeric RNA strands. The size of the dsRNA strands may vary from 8 nucleotides up to 20000 nucleotides independently for each strand.

In a preferred embodiment, the RNA is a ssRNA. In a preferred embodiment, the RNA is a ssRNA consisting of one single strand of RNA. Single stranded RNA can exist as minus strand [(−) strand] or as plus strand [(+) strand]. The (+) strand is the strand that comprises or encodes genetic information. The genetic information may be for example a nucleic acid sequence encoding a protein or polypeptide. When the (+) strand RNA encodes a protein, the (+) strand may serve directly as template for translation (protein synthesis). The (−) strand is the complement of the (+) strand. In the case of ssRNA, (+) strand and (−) strand are two separate RNA molecules. (+) strand and (−) strand RNA molecules may associate with each other to form a double-stranded RNA (“duplex RNA”).

In another aspect, the present invention provides a polyplex comprising a conjugate of Formula I*, preferably of Formula I, and a RNA, wherein said RNA is preferably non-covalently bound to said conjugate

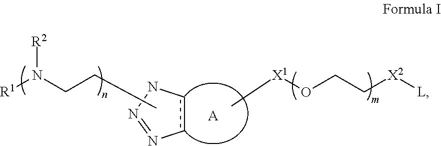

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

In a preferred embodiment, size of the RNA strand may vary from 8 nucleotides up to 20000 nucleotides.

In a preferred embodiment, said RNA is a ssRNA. In a preferred embodiment, said ssRNA is a mRNA. In a preferred embodiment, said RNA is a mRNA. In another preferred embodiment said nucleic acid is a DNA, wherein preferably said DNA is a plasmid DNA.

In another aspect, the present invention provides a polyplex comprising a conjugate of Formula I*, preferably of Formula I, and a mRNA, wherein said mRNA is preferably non-covalently bound to said conjugate

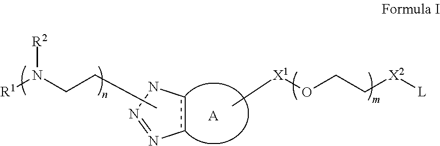

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

In a preferred embodiment, said RNA is a “messenger-RNA” (mRNA). In preferred embodiments, the term mRNA relates to a RNA transcript which encodes a peptide or protein. mRNA may be modified by stabilizing modifications and capping. Typically, a mRNA comprises a 5′ untranslated region (5′-UTR), a protein coding region, and a 3′ untranslated region (3′-UTR). Preferably, mRNA, in particular synthetic mRNA, contains a 5′ cap, UTRs embracing the coding region and a 3′ poly(A) tail. In one embodiment, the mRNA is produced by in vitro transcription using a DNA template where DNA refers to a nucleic acid that contains deoxyribonucleotides. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 3′-UTR, if present, is preferably located at the 3′ end of a gene, downstream of the termination codon of a protein-encoding region, but the term “3′-UTR” does preferably not include the poly(A) tail. Thus, the 3′-UTR is preferably upstream of the poly(A) tail (if present), e.g. directly adjacent to the poly(A) tail. A 5′-UTR, if present, is preferably located at the 5′ end of a gene, upstream of the start codon of a protein-encoding region. A 5′-UTR is preferably downstream of the 5′-cap (if present), e.g. directly adjacent to the 5′-cap. 5′- and/or 3′-untranslated regions may, according to the invention, be functionally linked to an open reading frame, so as for these regions to be associated with the open reading frame in such a way that the stability and/or translation efficiency of the RNA comprising said open reading frame are increased. The terms “poly(A) sequence” or “poly(A) tail” refer to an uninterrupted or interrupted sequence of adenylate residues which is typically located at the 3′ end of an RNA molecule. An uninterrupted sequence is characterized by consecutive adenylate residues. While a poly(A) sequence is normally not encoded in eukaryotic DNA, but is attached during eukaryotic transcription in the cell nucleus to the free 3′ end of the RNA by a template-independent RNA polymerase after transcription, the present invention also encompasses poly(A) sequences encoded by DNA. Terms such as “5′-cap”, “cap”, “5′-cap structure”, or “cap structure” are used synonymously and refer preferably to a nucleotide modification at the 5′ end of the mRNA, more preferably to a dinucleotide that is found on the mRNA 5′ end. A 5′-cap can be a structure wherein a (optionally modified) guanosine is bonded to the first nucleotide of an mRNA molecule via a 5′ to 5′ triphosphate linkage (or modified triphosphate linkage in the case of certain cap analogs). The term cap can refer to a naturally occurring cap or modified cap. RNA molecules may be characterized by a 5′-cap, a 5′-UTR, a 3′-UTR, a poly(A) sequence, and/or adaptation of the codon usage. The mRNA may be generated by chemical synthesis, in vivo or in vitro transcription, e.g. from a DNA or other nucleic acid template, or it may be recombinantly prepared or viral RNA. The mRNA includes non-self-amplifying mRNAs, such as endogenous mRNAs of mammalian cells, and self-amplifying mRNAs. Endogenous mRNA includes pre-mature and mature mRNA. The mRNA is preferably exogenous mRNA that has to enter the cell from outside the cell, e.g. by directly passing through the cytoplasmic membrane or by endocytosis followed by endosomal escape. mRNA preferably does not enter the nucleus, nor integrates into the genome. In a preferred embodiment, said mRNA have a size of bout and more than 100 nucleotides up to 20000 nucleotides.

The formation of the inventive polyplex is typically caused by electrostatic interactions between positive charges on side of the inventive conjugate and negative charges on side of the polyanion, nucleic acid and RNA respectively. This results in complexation and spontaneous formation of polyplexes. In one embodiment, an inventive polyplex refers to a particle having a z-average diameter suitable for parental administration.

In a preferred embodiment, said nucleic acid is a single stranded RNA (ssRNA). In a preferred embodiment, said ssRNA is a messenger RNA (mRNA). In a further preferred embodiment, said mRNA encodes a peptide or protein of interest. In a further preferred embodiment, said mRNA encodes a peptide or protein of interest, wherein said peptide or protein of interest is selected from reporter proteins and pharmaceutically active peptides or proteins. In a further preferred embodiment, said mRNA encodes a peptide or protein of interest, wherein said peptide or protein of interest is a reporter protein. In a further preferred embodiment, said mRNA encodes a peptide or protein of interest, wherein said peptide or protein of interest is a pharmaceutically active peptide or protein. In a further preferred embodiment, said mRNA is a pharmaceutically active nucleic acid. In a further preferred embodiment, said mRNA is a pharmaceutically active nucleic acid, wherein said pharmaceutically active nucleic acid is a nucleic acid that encodes a pharmaceutically active peptide or protein.

In another preferred embodiment said polyanion is a nucleic acid, wherein said nucleic acid is a DNA, wherein preferably said DNA is a plasmid DNA. In another preferred embodiment, said nucleic acid is a DNA. In a further preferred embodiment, said DNA is a plasmid DNA (pDNA). In a further preferred embodiment, said pDNA encodes a peptide or protein of interest. In a further preferred embodiment, said pDNA encodes a peptide or protein of interest, wherein said peptide or protein of interest is selected from reporter proteins and pharmaceutically active peptides or proteins. In a further preferred embodiment, said pDNA encodes a peptide or protein of interest, wherein said peptide or protein of interest is a reporter protein. In a further preferred embodiment, said pDNA encodes a peptide or protein of interest, wherein said peptide or protein of interest is a pharmaceutically active peptide or protein. In a further preferred embodiment, said pDNA is a pharmaceutically active nucleic acid. In a further preferred embodiment, said pDNA is a pharmaceutically active nucleic acid, wherein said pharmaceutically active nucleic acid is a nucleic acid that encodes a pharmaceutically active peptide or protein.

In a preferred embodiment, said nucleic acid is a nucleic acid that encodes a peptide or protein of interest. In a further preferred embodiment, said nucleic acid encodes a peptide or protein of interest, wherein said peptide or protein of interest is selected from reporter proteins and pharmaceutically active peptides or proteins. In a further preferred embodiment, said nucleic acid encodes a peptide or protein of interest, wherein said peptide or protein of interest is a reporter protein. In a further preferred embodiment, said nucleic acid encodes a peptide or protein of interest, wherein said peptide or protein of interest is a pharmaceutically active peptide or protein. In a further preferred embodiment, said nucleic acid is a pharmaceutically active nucleic acid. In a further preferred embodiment, said nucleic acid is a pharmaceutically active nucleic acid, wherein said pharmaceutically active nucleic acid is pharmaceutically active in its own. In a further preferred embodiment, said nucleic acid is a pharmaceutically active nucleic acid, wherein said pharmaceutically active nucleic acid is a nucleic acid that encodes a pharmaceutically active peptide or protein. In a further preferred embodiment, said pharmaceutically active nucleic acid is a mRNA. In a further preferred embodiment, said pharmaceutically active nucleic acid is a pDNA.

In a preferred embodiment, said nucleic acid encodes a peptide or protein of interest, wherein said peptide or protein of interest is a reporter protein. In these embodiments, the nucleic acid comprises a reporter gene. Certain genes may be chosen as reporters because the characteristics they confer on cells or organisms expressing them may be readily identified and measured, or because they are selectable markers. Reporter genes are often used as an indication of whether a certain gene has been taken up by or expressed in the cell or organism population. Preferably, the expression product of the reporter gene is visually detectable. Common visually detectable reporter proteins typically possess fluorescent or luminescent proteins. Examples of specific reporter genes include the gene that encodes jellyfish green fluorescent protein (GFP), which causes cells that express it to glow green under blue light, the enzyme luciferase, which catalyzes a reaction with luciferin to produce light, and the red fluorescent protein (RFP) as well as the ones known by the skilled person as described in Concilio S C et al., Molecular Therapy: Oncolytics, 2021, 21:98-109, incorporated herein by way of reference. Variants of any of these specific reporter genes are possible, as long as the variants possess visually detectable properties. For example, eGFP is a point mutant variant of GFP. In a preferred embodiment, said RNA is coding RNA, i.e. RNA encoding a peptide or protein. Said RNA may express the encoded peptide or protein. In a very preferred embodiment, said RNA, ssRNA or encoding RNA is a “messenger-RNA” (mRNA).

In a preferred embodiment, said RNA is a pharmaceutically active RNA. A “pharmaceutically active RNA” is an RNA that encodes a pharmaceutically active peptide or protein or is pharmaceutically active in its own, e.g., it has one or more pharmaceutical activities such as those described for pharmaceutically active proteins, e.g., immunostimulatory activity.

The term “encoding” refers to the inherent property of specific sequences of nucleotides in a RNA, such as an mRNA, to serve as templates for synthesis of other polymers and macromolecules in biological processes having either a defined sequence of nucleotides or a defined sequence of amino acids and the biological properties resulting therefrom. Thus, a gene encodes a protein if transcription and translation of mRNA corresponding to that gene produces the protein in a cell or other biological system. The terms “RNA encodes” or “RNA encoding”, as interchangeably used, means that the RNA, preferably the mRNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g. in the cytoplasm), may secrete the encoded peptide or protein, or may produce it on the surface.

With respect to RNA, and in particular with respect to mRNA, the term “expression” or “translation” relates to the process, typically in the ribosomes of a cell, by which a strand of mRNA directs the assembly of a sequence of amino acids to make a peptide or protein. The term “expression” is used in its most general meaning and comprises production of RNA and/or protein.

In another aspect, the present invention provides a polyplex comprising a conjugate of Formula I*, preferably of Formula I, and a pDNA, wherein said pDNA is preferably non-covalently bound to said conjugate

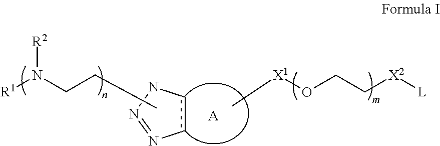

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

In a preferred embodiment, said pDNA is coding DNA, i.e. DNA encoding a peptide or protein. Said pDNA may express the encoded peptide or protein. In a very preferred embodiment, said pDNA is coding DNA expressing the encoded peptide or protein.

In a preferred embodiment, said pDNA is a pharmaceutically active pDNA. A “pharmaceutically active pDNA” is an pDNA that encodes a pharmaceutically active peptide or protein or is pharmaceutically active in its own, e.g., it has one or more pharmaceutical activities such as those described for pharmaceutically active proteins, e.g., immunostimulatory activity.

As known by the skilled person, for said preferred embodiments, such a double-stranded (ds) circular plasmid, i.e. a plasmid DNA, encoding peptide or protein of interest, preferably a pharmaceutically active peptide or protein, consists of, at minimum, a promoter and a gene of interest encoding said peptide or protein of interest, preferably said pharmaceutically active peptide or protein, and typically and preferably comprises further control elements such as appropriate promoters and terminators operably linked to said gene of interest encoding said pharmaceutically active peptide or protein.

A “pharmaceutically active peptide or protein” or “therapeutic peptide or protein” is a peptide or a protein that has a positive or advantageous effect on a condition or disease state of a subject when provided to the subject in a therapeutically effective amount. In one embodiment, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or to lessen the severity of such disease or pathological condition. The term “pharmaceutically active peptide or protein” includes entire proteins or polypeptides, and can also refer to pharmaceutically active fragments thereof. It can also include pharmaceutically active analogs of a peptide or protein. The term “pharmaceutically active peptide or protein” includes peptides and proteins that are antigens, i.e., the peptide or protein elicits an immune response in a subject which may be therapeutic or partially or fully protective. In one embodiment, the pharmaceutically active peptide or protein is or comprises an immunologically active compound or an antigen or an epitope.

As used herein, the terms “effective amount” and “therapeutically effective amount” are used interchangeably and refer to an amount administered to a subject, either as a single dose or as part of a series of doses, which is effective to produce a desired physiological response or desired therapeutic effect in the subject. Examples of desired therapeutic effects include, without limitation, improvements in the symptoms or pathology, and/or reducing the progression of symptoms or pathology in a subject suffering from an infection, disease, disorder and/or condition; and/or slowing, preventing or delaying the onset of symptoms or pathology of an infection, disease, disorder and/or condition in a subject susceptible to said infection, disease, disorder and/or condition. The therapeutically effective amount will vary depending on the nature of the formulation used and the type and condition of the recipient. The determination of appropriate amounts for any given composition is within the skill in the art, through standard tests designed to assess appropriate therapeutic levels. Typical and preferred therapeutically effective amounts of the inventive triconjugates and/or polyplexes described herein range from about 0.05 to 1000 mg/kg body weight, and in particular from about 5 to 500 mg/kg body weight.

Thus, in another aspect, the present invention provides a polyplex comprising a conjugate of Formula I*, preferably of Formula I, and a RNA, wherein said RNA is preferably non-covalently bound to said conjugate, and wherein said RNA is a pharmaceutically active RNA.

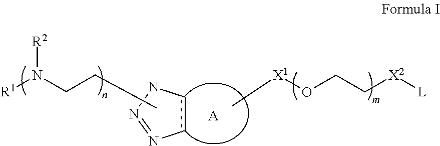

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

In another aspect, the present invention provides a polyplex comprising a conjugate of Formula I*, preferably of Formula I, and a RNA, wherein said RNA is preferably non-covalently bound to said conjugate, and wherein said RNA is a pharmaceutically active RNA encoding a pharmaceutically active peptide or protein.

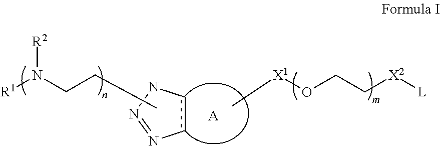

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

In a preferred embodiment, said RNA encoding a pharmaceutically active peptide or protein has a size of 100 to about 20000 nucleotides.

In a preferred embodiment, said pharmaceutically active peptide or protein is or comprises an immunologically active compound or an antigen or an epitope. In a preferred embodiment, said pharmaceutically active peptide or protein is or comprises an immunologically active compound or an antigen. In a preferred embodiment, said pharmaceutically active peptide or protein is or comprises an immunologically active compound. In a preferred embodiment, said pharmaceutically active peptide or protein is or comprises an antigen. In a preferred embodiment, said pharmaceutically active peptide or protein is or comprises an epitope.

The term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells, or inducing degranulation of immune cells such as mast cells, eosinophils, neutrophils, cytotoxic T cells or NK cells. In one embodiment, the immune response involves stimulation of an antibody response (usually including immunoglobulin G (IgG)) and/or a cellular response including but not limited to responses by T cells, dendritic cells (DCs), macrophages, natural killer (NK) cells, natural killer T cells (NKT) cells, and γδ T cells. Immunologically active compounds may possess potent immunostimulating activity including, but not limited to, antiviral and antitumor activity, and can also down-regulate other aspects of the immune response, for example shifting the immune response away from a Th2 immune response, which is useful for treating a wide range of Th2 mediated diseases, or, if appropriate, shifting the immune response away from a Th1 immune response.

The term “antigen” covers any substance that will elicit an immune response. In particular, an “antigen” relates to any substance that reacts specifically with antibodies or T-lymphocytes (T-cells). The term “antigen” comprises any molecule which comprises at least one epitope, preferably against which an immune response can be generated. Preferably, an antigen in the context of the present invention is a molecule which, optionally after processing, induces an immune reaction, which is preferably specific for the antigen, including wherein the immune reaction may be both a humoral as well as a cellular immune reaction. The antigen is preferably presented by a cell, preferably by an antigen presenting cell, in the context of MHC molecules, which results in an immune reaction against the antigen. Antigens include or may be derived from allergens, viruses, bacteria, fungi, plants, parasites and other infectious agents and pathogens or an antigen may also be a tumor antigen. In preferred embodiments, the antigen is a surface polypeptide, i.e. a polypeptide naturally displayed on the surface of a cell, a pathogen, a bacterium, a virus, a fungus, a plant, a parasite, an allergen, or a tumor. The antigen may elicit an immune response against a cell, a pathogen, a bacterium, a virus, a fungus, a plant, a parasite, an allergen, or a tumor.

In one embodiment, an antigen is a self-antigen or a non-self-antigen. In another embodiment, said non-self-antigen is a bacterial antigen, a virus antigen, a fungus antigen, an allergen or a parasite antigen. It is preferred that the antigen comprises an epitope that is capable of eliciting an immune response in a target organism. For example, the epitope may elicit an immune response against a bacterium, a virus, a fungus, a parasite, an allergen, or a tumor. In some embodiments the non-self-antigen is a bacterial antigen.

In some embodiments the non-self-antigen is a virus antigen. In some embodiments the non-self-antigen is a polypeptide or a protein from a fungus. In some embodiments the non-self-antigen is a polypeptide or protein from a unicellular eukaryotic parasite.

In some embodiments the antigen is a self-antigen, particularly a tumor antigen. Tumor antigens and their determination are known to the skilled person. In the context of the present invention, the term “tumor antigen” or “tumor-associated antigen” relates to proteins that are under normal conditions specifically expressed in a limited number of tissues and/or organs or in specific developmental stages, for example, the tumor antigen may be under normal conditions specifically expressed in stomach tissue, preferably in the gastric mucosa, in reproductive organs, e.g., in testis, in trophoblastic tissue, e.g., in placenta, or in germ line cells, and are expressed or aberrantly expressed in one or more tumor or cancer tissues. In this context, “a limited number” preferably means not more than 3, more preferably not more than 2. The tumor antigens in the context of the present invention include, for example, differentiation antigens, preferably cell type specific differentiation antigens, i.e., proteins that are under normal conditions specifically expressed in a certain cell type at a certain differentiation stage, cancer/testis antigens, i.e., proteins that are under normal conditions specifically expressed in testis and sometimes in placenta, and germ line specific antigens. The tumor antigen is preferably associated with the cell surface of a cancer cell and is preferably not or only rarely expressed in normal tissues. Preferably, the tumor antigen or the aberrant expression of the tumor antigen identifies cancer cells. The tumor antigen that is expressed by a cancer cell in a subject, e.g., a patient suffering from a cancer disease, is preferably a self-protein in said subject. In preferred embodiments, the tumor antigen is expressed under normal conditions specifically in a tissue or organ that is non-essential, i.e., tissues or organs which when damaged by the immune system do not lead to death of the subject, or in organs or structures of the body which are not or only hardly accessible by the immune system. Preferably, the amino acid sequence of the tumor antigen is identical between the tumor antigen which is expressed in normal tissues and the tumor antigen which is expressed in cancer tissues. In a preferred embodiment, said term “tumor antigen” refers to a constituent of cancer cells which may be derived from the cytoplasm, the cell surface and the cell nucleus, preferably it refers to those antigens which are produced intracellularly or as surface antigens on tumor cells.

In a preferred embodiment, said nucleic acid is a pharmaceutically active nucleic acid. A “pharmaceutically active nucleic acid” is a nucleic acid that encodes a pharmaceutically active peptide or protein or is pharmaceutically active in its own, e.g., it has one or more pharmaceutical activities such as those described for pharmaceutically active proteins, e.g., immunostimulatory activity.

In another aspect, the present invention provides a polyplex comprising a conjugate of Formula I*, preferably of Formula I, and a nucleic acid, wherein said nucleic acid is preferably non-covalently bound to said conjugate, and wherein said nucleic acid is a pharmaceutically active nucleic acid.

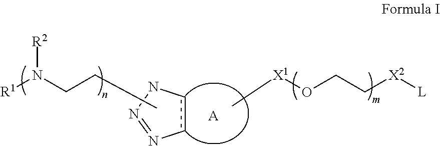

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

In another aspect, the present invention provides a polyplex comprising a conjugate of Formula I*, preferably of Formula I, and a nucleic acid, wherein said nucleic acid is preferably non-covalently bound to said conjugate, and wherein said nucleic acid is a pharmaceutically active nucleic acid encoding a pharmaceutically active peptide or protein.

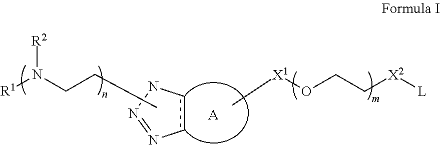

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

Examples of said pharmaceutically active peptides or proteins include, but are not limited to, cytokines and derivatives thereof such as cytokine-fusions (like albumin-cytokine fusions) and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, T cell receptors, chimeric antigen receptors (CARs), immunoglobulins including antibodies or bi-, tri-, or multispecific antibodies, e.g., for immune stimulation or production of neutralizing antibodies in case of viral/bacterial infection, soluble major histocompatibility complex antigens, immunologically active antigens such as bacterial, parasitic, or viral antigens, allergens, autoantigens, antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases, lysosomal enzymes and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, Von Willebrand factor, antithrombin III, glucocerebrosidase, erythropoietin granulocyte colony stimulating factor (GCSF) or modified Factor VIII, anticoagulants and the like.

In a preferred embodiment, said pharmaceutically active peptide or protein is selected from cytokines and derivatives thereof such as cytokine-fusions (like albumin-cytokine fusions) and immune system proteins such as immunologically active compounds (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, seletins, homing receptors, T cell receptors, chimeric antigen receptors (CARs), immunoglobulins including antibodies or bispecific antibodies, e.g., for immune stimulation or production of neutralizing antibodies in case of viral/bacterial infection, soluble major histocompatibility complex antigens, immunologically active antigens such as bacterial, parasitic, plant or viral antigens, allergens, autoantigens, antibodies), hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylate cyclases, neuramidases, lysosomal enzymes and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, Von Willebrand factor, antithrombin III, glucocerebrosidase, erythropoietin granulocyte colony stimulating factor (GCSF) or modified Factor VIII, anticoagulants and the like.

In a preferred embodiment, said pharmaceutically active peptide or protein is a immunologically active compound. In a preferred embodiment, said pharmaceutically active peptide or protein is a immunologically active compound selected from interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, T cell receptors, chimeric antigen receptors (CARs), immunoglobulins including antibodies or bispecific antibodies, e.g., for immune stimulation or production of neutralizing antibodies in case of viral/bacterial infection, soluble major histocompatibility complex antigens, immunologically active antigens such as bacterial, parasitic, plant or viral antigens, allergens, autoantigens and antibodies. In another preferred embodiment, said pharmaceutically active peptide or protein is an interleukin. In another preferred embodiment, said pharmaceutically active peptide or protein is a colony stimulating factor (CSF). In another preferred embodiment, said pharmaceutically active peptide or protein is a granulocyte colony stimulating factor (G-CSF). In another preferred embodiment, said pharmaceutically active peptide or protein is a granulocyte-macrophage colony stimulating factor (GM-CSF). In another preferred embodiment, said pharmaceutically active peptide or protein is erythropoietin. In another preferred embodiment, said pharmaceutically active peptide or protein is tumor necrosis factor (TNF). In another preferred embodiment, said pharmaceutically active peptide or protein is an interferons. In another preferred embodiment, said pharmaceutically active peptide or protein is an integrin. In another preferred embodiment, said pharmaceutically active peptide or protein is an addressin. In another preferred embodiment, said pharmaceutically active peptide or protein is a selectin. In another preferred embodiment, said pharmaceutically active peptide or protein is an immunologically active antigen, preferably selected from bacterial, parasitic, plant or viral antigens, allergens, autoantigens and antibodies. In another preferred embodiment, said pharmaceutically active peptide or protein is a bacterial antigen. In another preferred embodiment, said pharmaceutically active peptide or protein is a parasitic antigen. In another preferred embodiment, said pharmaceutically active peptide or protein is a plant antigen. In another preferred embodiment, said pharmaceutically active peptide or protein is a viral antigen. In another preferred embodiment, said pharmaceutically active peptide or protein is an allergen. In another preferred embodiment, said pharmaceutically active peptide or protein is an autoantigen. In another preferred embodiment, said pharmaceutically active peptide or protein is an antibody.

In another preferred embodiment, said pharmaceutically active peptide or protein is selected from interleukin-2, interleukin-4, interleukin-7, interleukin-12, interleukin-15, interferon-α, interferon-β, interferon-γ, colony stimulating factor, granulocyte-macrophage stimulating factor, anti-angiogenic agents, tumor suppressor genes, tumor antigens, viral antigens and bacterial antigens.

In a preferred embodiment, said pharmaceutically active peptide or protein is selected from a cytokine, a growth factor, a hormone, an enzyme, a tumor antigen, a viral antigen, bacterial antigen, an autoantigen, or an allergen.

In a preferred embodiment, said pharmaceutically active peptide or protein comprises, preferably consists of a cytokine.

The term “cytokine”, as used herein, refers to a category of small proteins (about 5-20 kDa) that are important in cell signalling. Their release has an effect on the behavior of cells around them. Cytokines are involved in autocrine signalling, paracrine signalling and endocrine signalling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

In a preferred embodiment, said pharmaceutically active peptide or protein is a cytokine selected from an interleukin, an interferon and a chemokine.

In a preferred embodiment, said pharmaceutically active peptide or protein is an interleukin. In a further preferred embodiment, said pharmaceutically active peptide or protein is an interleukin selected from the group consisting of IL-2, IL-7, IL-12, IL-15, and IL-21.

In a further preferred embodiment, said pharmaceutically active peptide or protein is interleukin-2 (IL-2).

Interleukin-2 (IL-2), a key cytokine with pleiotropic effects on the immune system, is produced mainly by antigen-stimulated CD4⁺ T cells, as well as by CD8⁺ T cells, natural killer (NK) and dendritic cells (DC). IL-2 promotes the differentiation of naïve CD4 T cells into T helper-1 (Th1) and T helper-2 (Th2) cells and is required for the maintenance of CD4⁺ regulatory T cells (Tregs). Furthermore, IL-2 promotes CD8 T cell and NK cell cytotoxicity (Liao W et al, Immunity, 2013, 38(1):13-25).

The IL-2 receptor is composed of the three subunits IL-2Rα (CD25), IL-2Rβ (CD122), and IL-2Rγ (CD132). IL-2Rα is unique to IL-2 and is expressed by several immune cells including Tregs, activated CD4 and CD8 T cells, B cells and mature Dendritic cells (Wrangle J M et al, J Interferon Cytokine Res, 2018, 38(2):45-68). Binding of IL-2 to the IL-2Rβγ or IL-2Rαβγ complex leads to the recruitment of Janus family tyrosine kinases (JAK1, JAK3), phosphorylation of signal transducer and activator of transcription (STAT1, STAT3, STAT5) and activation of major downstream signaling pathways, which regulate survival, proliferation, differentiation, activation, cytokine production in different types of immune cells (Wrangle J M et al, J Interferon Cytokine Res, 2018, 38(2):45-68).

Recombinant IL-2 protein was approved by FDA in 1998 for treatment of metastatic melanoma and renal cancer. Although IL-2 mediates tumor regression, it fails to improve patients' survival and is associated with severe toxicity (Wrangle J M et al, J Interferon Cytokine Res, 2018, 38(2):45-68; Jiang T et al., Oncoimmunology, 2016, 5(6):e1163462). Due to rapid elimination and metabolism via the kidney, IL-2 has a short serum half-life of several minutes. Thus, to achieve an optimal immune-modulatory effect, IL-2 should be given in a high dose, which will inevitably result in severe toxicities.

Therefore, the present invention to target the delivery of mRNA or plasmid DNA encoding IL2 protein will allow its protein expression at the tumor site. This will enable the activation of the immune cells at the tumor microenvironment and allows to induce an anti-tumorigenic effect with potential limited toxicity.

In a preferred embodiment, said pharmaceutically active peptide or protein is an interferon.

In a preferred embodiment, said pharmaceutically active peptide or protein is an interferon, wherein said interferon is a type-I interferon.

In a preferred embodiment, said pharmaceutically active peptide or protein is an interferon, wherein said interferon is a type-II interferon.

In a preferred embodiment, said pharmaceutically active peptide or protein is an interferon, wherein said interferon is interferon-α (IFN-α), interferon-β (IFN-0), or interferon-γ (IFN-γ).

In a preferred embodiment, said pharmaceutically active peptide or protein is an interferon, wherein said interferon is interferon-α (IFN-α).

In a preferred embodiment, said pharmaceutically active peptide or protein is an interferon, wherein said interferon is interferon-β (IFN-0).

Type-I interferons (IFNs) were originally identified by their anti-viral effects; however, they play important roles in other diseases, including cancer and multiple sclerosis. IFNs have pleiotropic anti-cancer effects, acting on cancer cells both directly and indirectly. Indirect effects include activation of immune effector cells and ablation of the tumor vasculature (Borden, E. C., Nat Rev Drug Discov, 2019, 18:219-234).

IFNs are a subset of the class-2 α-helical cytokines that have been found in all vertebrates.

There are numerous human Type-I-IFNs, including thirteen IFN-α cytokines, one IFN—P, and several other single gene products not yet well characterized (Musella M et al., Oncoimmunology, 2017 6(5): e1314424).

In their canonical signaling pathway, Type-I-IFNs bind to the heterodimeric transmembrane IFN-α/P receptor (IFNAR), which activates the Janus Kinase-Signal Transducer and Activator of Transcription (JAK-STAT) pathway. This cascade induces the transcription of several hundred IFN-stimulated genes (ISGs), resulting in multilayered cellular responses (Schneider W M et al., Annu Rev Immunol, 2014, 32:513-545). These include protein synthesis (both cellular and viral), autophagy, apoptosis, angiogenesis, and immune cell modulation (Borden, E. C., Nat Rev Drug Discov, 2019, 18:219-234). Type I IFNs modulate the activity of both innate and adaptive immune cells, including dendritic cells, CD8+ T cells, CD4+ T cells, Regulatory T cells, and NK cells (Boukhaled G M et al., Annu Rev Pathol, 2021, 16:167-198).

Type I IFNs have been widely used, alone and in combination with other immunotherapeutic agents, to treat solid and hematologic malignancies (Borden, E. C., Nat Rev Drug Discov, 2019, 18:219-234). IFN-α2 was the first human immunotherapeutic approved by the US Food and Drug Administration (FDA) for cancer treatment and is still commonly combined with IL-2 in immunotherapeutic regimens for metastatic renal-cell carcinomas and cutaneous melanoma. IFN-β exerts antiviral and antiproliferative properties similar to those of IFN-α and is available as a preparation derived from natural fibroblasts (IFN-β 1a) or in recombinant form (IFN-β 1b). Nonetheless, systemic administration of IFNs has been associated with severe toxicities. Therefore, optimal concentrations within the tumor bed after administration of tolerable doses of IFNs cannot be achieved (Young P A et al., Semin Oncol, 2014 41(5):623-636).

Beyond cancer treatment, IFN-β is the standard treatment for Multiple Sclerosis (MS). To date, five IFN-β drugs have been approved for the treatment of relapsing forms of MS (Filipi M et al., Int J MS Care, 2020, 22(4):165-172). Furthermore, IFN-β is used in Japan for the treatment of hepatitis C.

Targeted delivery of the nucleic acids encoding for IFN—P is expected to reduce the associated systemic toxicity. Moreover, targeted expression should lead to high, localized IFN—R concentrations in the desired tissue.

In a preferred embodiment, said pharmaceutically active peptide or protein is an interferon, wherein said interferon is IFN-γ.

Interferon-gamma (IFN-γ), a type II IFN, is a pleiotropic molecule which has antiproliferative, pro-apoptotic and antitumor immunomodulatory mechanisms of action (Castro F et al., Frontier in Immunology, 2018, 9:847). IFNγ is produced by the immune cells, including activated T cells and natural killer (NK) cells. IFNγ exerts its antitumor effects through the activation JAK-STAT pathway that leads to the expression of IFNγ-stimulated genes (ISGs) (Chen Y et al., Journal of Pancreatology, 2023, 6(1):8-17; Ding H et al., Biomed Pharmacother, 2022, 155:113683). IFNγ plays a role in maturation of NK cells, enhancement of CD8 T cell cytotoxicity, stimulation of Th1 polarization, inhibition of Th2 and Th17 differentiation, upregulation of MHC class I and II in APCs, maturation of Dendritic Cells, and the induction of M1 macrophages (Chen Y et al., Journal of Pancreatology, 2023, 6(1):8-17; Ding H et al., Biomed Pharmacother, 2022, 155:113683). The direct inhibitory effects of IFNγ on tumor cells include cell cycle arrest, cell senescence, apoptosis and autophagic cell death. Moreover, it has been demonstrated that IFNγ mediates inhibition of tumor associated fibroblasts in TME and induces an anti-angiogenic effect. Tumor cells limit the production of IFNγ by cytotoxic CD8 T cell by imposing nutrient deprivation or rewiring the cellular metabolism of T cells (Chen Y et al., Journal of Pancreatology, 2023, 6(1):8-17; Ding H et al., Biomed Pharmacother, 2022, 155:1 1368³). Overcoming primary or acquired resistance to IFNγ-induced therapies remains a great clinical challenge (Chen Y et al., Journal of Pancreatology, 2023, 6(1):8-17; Ding H et al., Biomed Pharmacother, 2022, 155:113683). Several clinical trials investigating recombinant IFNγ have shown some improvement in patient survival. However, most studies failed due to toxicity (Chen Y et al., Journal of Pancreatology, 2023, 6(1):8-17; Ding H et al., Biomed Pharmacother, 2022, 155:113683). Therefore, targeting the delivery of IFNγ mRNA and expressing it directly in tumor cells can therefore reduce the systemic toxicity and modulate the tumor microenvironment.

In a further preferred embodiment, said pharmaceutically active peptide or protein is a hormone.

In a further preferred embodiment, said pharmaceutically active peptide or protein is human erythropoietin (EPO).

The human erythropoietin (EPO) protein is a hormone that stimulates the production of red blood cells (erythropoiesis) in the bone marrow by binding to the EPO receptor of blood cell precursors, the proerythroblasts, stimulating their differentiation and inhibiting their apoptosis (McGraw K et al., Vitam Horm, 2017, 105:79-100). In adults, EPO is mainly produced by peritubular cells in the kidneys and to a much smaller extent by the liver, spleen, bone marrow, lung and brain (McGraw K et al., Vitam Horm, 2017, 105:79-100; Jelkmann W et al., Transfus Med Hemother, 2013, 40(5):302-309).

Recombinant human EPO (rhEPO) is used in the treatment of anemia associated with chronic kidney disease, HIV infection and chemotherapy and in perioperative therapies (Jelkmann W et al., Transfus Med Hemother, 2013, 40(5):302-309). Repeated administration of rhEPO is associated with high immunogenicity caused by the development of neutralizing antibodies against EPO (Casadevall N et al., N Engl J Med, 2002, 346(7):469-475; Behler C M et al., J Med Case Rep, 2009, 3:7335; Praditpornsilpa K et al., Nephrol Dial Transplant, 2009, 24(5):1545-1549; Rahbar M et al., J Nephropathol, 2017, 6(1):25-29). Recombinant proteins often have different patterns of glycosylation from the endogenously expressed protein, leading to the development of neutralizing antibodies against the rhEPO (Susantad T et al., Sci Rep, 2021, 11(1):1491). These antibodies can also target the endogenous EPO protein (Casadevall N et al., N Engl J Med, 2002, 346(7):469-475).

We have developed a novel approach to selectively deliver mRNAs which encode therapeutic proteins, such as EPO, and enable their endogenous expression in the targeted tissue cells in a living organism. This would allow a more physiological approach/natural source, as opposed to the systemic administration of recombinant EPO. This could also significantly improve the efficacy and safety as compared to the use of recombinant protein-based therapies, like rhEPO in the treatment of anemia. It could reduce immunogenicity as proteins will be expressed with the correct post-translational modifications.

In a further preferred embodiment, said pharmaceutically active peptide or protein is a bacterial antigen.

In a further preferred embodiment, said pharmaceutically active peptide or protein is a viral antigen.

In a further preferred embodiment, said pharmaceutically active peptide or protein is a tumor antigen.

In a further preferred embodiment, said pharmaceutically active peptide or protein is a plant antigen.

In a further preferred embodiment, said pharmaceutically active peptide or protein is Diphtheria toxin (DT).

In a further preferred embodiment, said pharmaceutically active peptide or protein is Diphtheria toxin catalytic domain A (DT-A).

Diphtheria toxin (DT) is one of the most studied bacterial exotoxins. DT is secreted by a non-encapsulated, non-motile, Gram-positive bacillus, Corynebacterium diphtheriae. DT is a single polypeptide chain comprising two major domains: the catalytic domain A (DT-A) and B subunit (DT-B). DT-A catalyses inactivation of elongation factor 2 through ADP-ribosylation, thereby blocking protein synthesis and cell death in the target cells (Falnes P O et al, EMBO J, 1998, 17(2):615-625). DT-B includes the translocation and receptor-binding regions and promotes the binding of the toxin to cells and the entry of the A chain into the cytosolic compartment, leading to cell death (Sharma N C et al., Nature Reviews Disease Primers, 2019, 5(1):81).

Several approaches utilizing DT as potential anti-cancer therapies have been examined in pre-clinical studies and clinical trials (Shafiee F et al., Front Microbiol, 2019, 10:2340). These include, Antibody-conjugated DT, ligand-targeted DT as well as gene therapies, whereby, the gene encoding the DT is delivered, to produce the toxin in vivo. Some of the DT agents that reached the clinical trials are Ontak™ and Tagraxofusp™ (Frankel A E et al., Biomedicines, 2019, 7(1):6; Frankel A E et al., Blood, 2007, 110(11):894).

As most cells possess the DT cell surface receptor, a non-targeted full-length DT could lead to significant toxicity. There is a need to discover a strategy harnessing the power of the DT in a targeted manner to cancer cells while minimizing off-target effects.

As a low level of DT-A is sufficient for cell killing, the development of specific targeting strategies for DT-A could result in fewer adverse effects on normal cells and tissues while achieving efficient cell killing (Yamaizumi, M et al., Cell, 1978, 1:245-250).

The present invention of targeted delivery of mRNA encoding DT-A will allow its expression at the tumor site and should result in higher efficiency in eradicating tumor cells with reduced systemic toxicity. Moreover, in immunized cancer patients, this strategy may offer additional benefits due to the pre-existing immunity to diphtheria toxin. The immune system could recognize and respond to the DT protein more efficiently, leading to a stronger immune response against the cancer cells. This could potentially increase the efficacy of the treatment.

In a further preferred embodiment, said pharmaceutically active peptide or protein is a receptor binding domain (RBD) of a coronavirus (CoV), or a fragment thereof.

In a further preferred embodiment, said pharmaceutically active peptide or protein is the receptor binding domain (RBD), preferably the receptor binding motif (RBM), of a spike (S) protein of a human coronavirus (HCoV), or a fragment thereof, wherein said HCoV is selected from SARS-CoV-2, SARS-CoV, MERS-CoV.

The spike protein (S) is a type I transmembrane protein expressed on the surface of coronaviruses that mediates the entrance of the virus by interacting with receptors on the target cells (angiotensin-converting enzyme 2, ACE2) (Walls A C et al., Cell, 2020, 181(2):281-292.e6). Coronavirus S proteins are composed of three copies of an S1 subunit and three copies of an S2 subunit. Within the S1 subunit, an N-terminal domain (NTD) and a receptor-binding domain (RBD) are present (Walls A C et al., Cell, 2020, 181(2):281-292.e6).

During the recent COVID-19 pandemic, monoclonal antibodies isolated from patients infected with the newly identified coronavirus (SARS-CoV-2) were characterized. The RBD was found to be the main target of neutralizing antibodies, but also the NTD and the quaternary structure of the trimer were recognized by strong neutralizing antibodies (Barnes C O et al., Cell, 2020, 182(4):828-842.e16; Heinz F X et al., NPJ Vaccines, 2021, 6(1):104). This suggests that a properly folded S protein is needed to induce a potent immune response.

mRNA-based vaccines encoding the S protein of SARS-CoV-2 have been developed, and these have been shown to induce strong and durable immune responses (Widge A T et al., N Engl J Med, 2021, 384(1):80-82; Sahin U et al., Nature, 2020, 586(7830):594-599 and Erratum in Nature, 2021, 590(7844):E17). Selectively delivering the mRNA encoding the spike protein to cancer cells would induce the immune system in the tumor microenvironment to target and destroy the transfected cancer cells. In patients already immunized against the protein, a particularly effective immune response against the cancer cells would be expected.

In a further aspect, the present invention provides for the use of pharmaceutical compositions as described herein and comprising the inventive polyplexes which polyplexes comprises said pharmaceutically active nucleic acid encoding a pharmaceutically active peptide or protein for the therapeutic or prophylactic treatment of various diseases, in particular diseases in which provision of said peptide or protein to a subject results in a therapeutic or prophylactic effect. For example, provision of an antigen or epitope which is derived from a virus may be useful in the treatment of a viral disease caused by said virus. Provision of a tumor antigen or epitope may be useful in the treatment of a cancer disease wherein cancer cells express said tumor antigen. Provision of a cytokine or a cytokine-fusion may be useful to modulate tumor microenvironment. Provision of cytokines, hormones or growth factors can be used for the treatment of non-oncology related diseases.

In a further aspect, the present invention provides a pharmaceutical composition comprising an inventive composition, an inventive conjugate, preferably said conjugate of Formula I* or of Formula I, or an inventive polyplex as described herein, and a pharmaceutically acceptable salt thereof.

### Negatively Charged Polyanions Used to Form Polyplexes

The triconjugates of the present disclosure can form polyplexes with polyanions and anionic polymers, such as nucleic acids. For example, at physiological pH (e.g., pH 7.4), the LPEI fragment of a triconjugate of the present invention can be at least partially protonated and can carry a net positive charge. In contrast, polyanions such nucleic acids can be at least partially deprotonated at physiological pH and can carry a net negative charge. Accordingly, in some embodiments co-incubation of a triconjugate of the present invention with a negatively charged polymer and polyanion such as a nucleic acid, and preferably a RNA, further preferably a mRNA, or a pDNA, will result in a polyplex (e.g., held together by electrostatic interaction).

In another aspect, the present invention provides a polyplex comprising a conjugate of Formula I*, preferably of Formula I, and a nucleic acid, wherein preferably said nucleic acid is a mRNA or a pDNA, wherein said nucleic acid, preferably said mRNA or said pDNA is preferably non-covalently bound to said conjugate

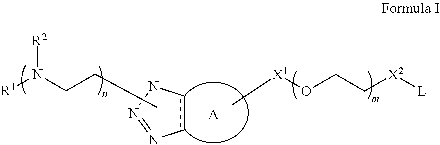

wherein A, R¹, R², X¹, X², L, m and n are as defined herein, preferably as defined in any embodiment described herein, be it individually related to each parameter A, R¹, R², X¹, X², L, m and n, or collectively to some or all of A, R¹, R², X¹, X² μL, m and n.

In another aspect, the present invention provides a polyplex comprising a conjugate of Formula I, or a pharmaceutically acceptable salt, solvate, hydrate, tautomer or enantiomer thereof, and a nucleic acid, wherein preferably said nucleic acid is a mRNA:

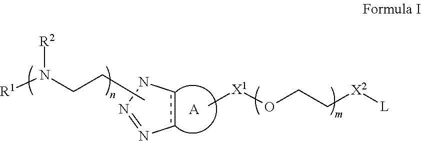

wherein:
[symbol] is a single bond or a double bond;
n is any integer between 1 and 1500;
m is a discrete number of repeating units m of 2 to 100, preferably of a discrete number of repeating units m of 4 to 60;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ— is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H; X¹ is a divalent covalent linking moiety; X² is a divalent covalent linking moiety; and L is a targeting fragment, wherein preferably said targeting fragment is capable of binding to a cell, and wherein further preferably said targeting fragment is capable of binding to a cell surface receptor. In a preferred embodiment, said R¹ is —H. In a preferred embodiment, said R¹ is —CH₃.

In a preferred embodiment, said Ring A is cyclooctene, succinimide, or 7- to 8-membered heterocycloalkenyl, wherein the heterocycloalkyl or heterocycloalkenyl comprises one or two heteroatoms selected from N, O and S, and wherein each cyclooctene, heterocycloalkyl or heterocycloalkenyl is optionally substituted at any position with one or more R^{A1}, wherein preferably R^{A1} is oxo or fluorine, or wherein two R^{A1} combine to form one or more fused phenyl rings, preferably one or two fused phenyl rings, wherein each phenyl ring is optionally substituted with one or more —SO₃H or —OSO₃H.

In a preferred embodiment of any aspects of the present disclosure and invention, said conjugate of Formula I is a conjugate selected from:

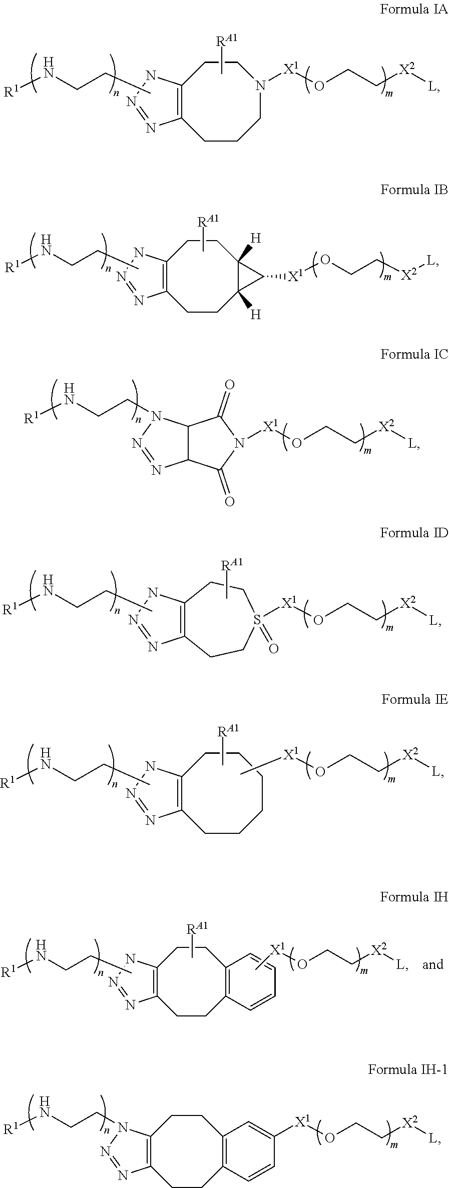

wherein wherein R¹, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment of any aspects of the present disclosure and invention, said conjugate of Formula I is a conjugate selected from:

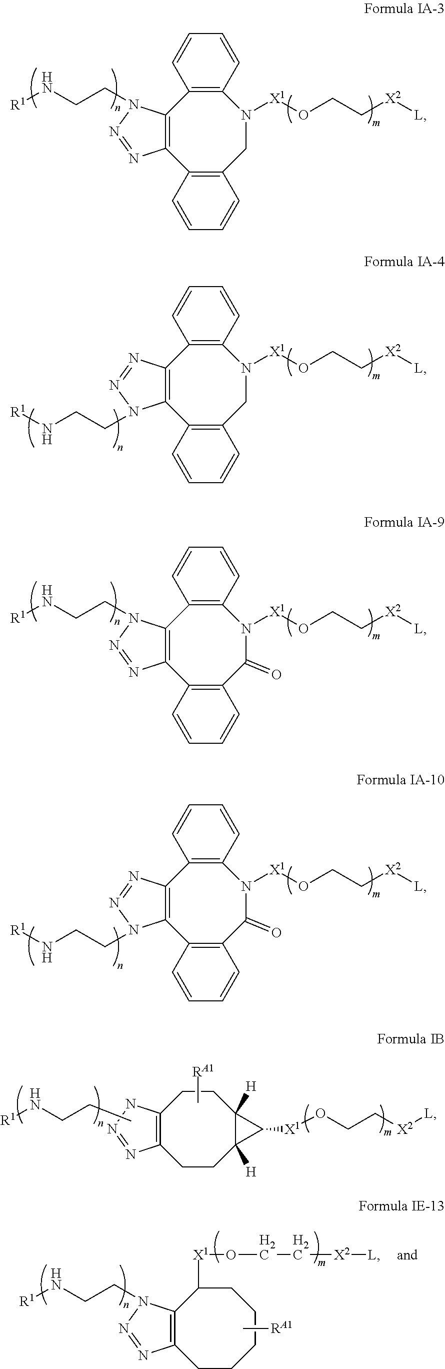

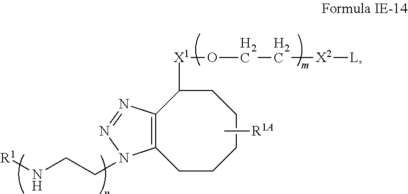

wherein R¹, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment of any aspects of the present disclosure and invention, said conjugate of Formula I is a conjugate selected from:

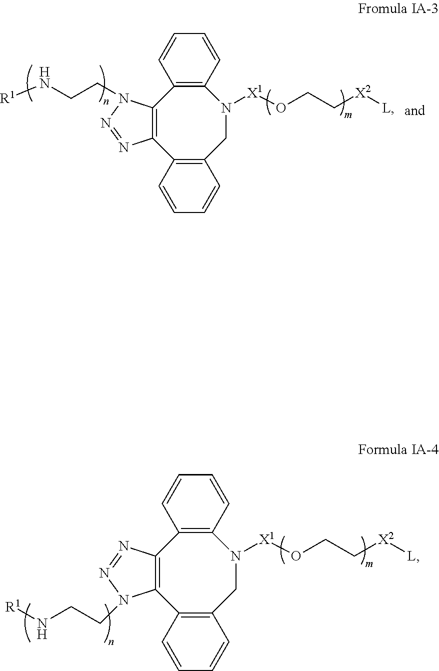

wherein R¹, X¹, X², L, m and n are as defined herein, preferably as defined in any embodiment described herein, be it individually related to each parameter R¹, X¹, X², L, m and n, or collectively to some or all of R¹, X¹, X², L, m and n.

In a preferred embodiment of any aspects of the present disclosure and invention, said conjugate of Formula I is a conjugate selected from:

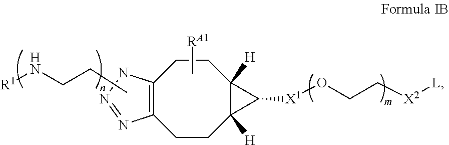

wherein R¹, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment of any aspects of the present disclosure and invention, said conjugate of Formula I is a conjugate selected from:

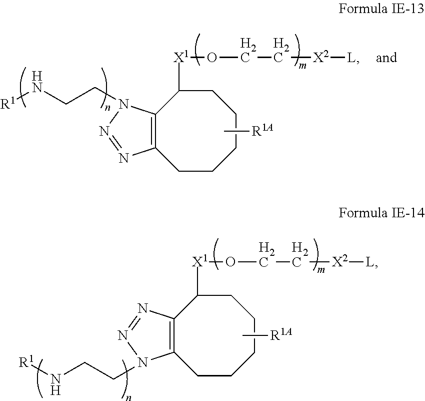

wherein R, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another preferred embodiment, said targeting fragment is epidermal growth factor (EGF), and wherein preferably said targeting fragment is human EGF (hEGF), and wherein again further preferably said targeting fragment comprises, preferably consists of, the sequence of SEQ ID NO:7. In another aspect, the present invention provides a polyplex comprising a conjugate of Formula I, or a pharmaceutically acceptable salt, solvate, hydrate, tautomer or enantiomer thereof, and a nucleic acid, wherein preferably said nucleic acid is a pDNA, wherein said nucleic acid, preferably said pDNA is preferably non-covalently bound to said conjugate:

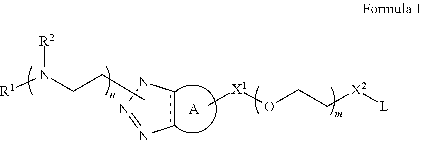

wherein:
[symbol] is a single bond or a double bond;
n is any integer between 1 and 1500;
m is a discrete number of repeating units m of 36;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)— is H;
Ring A is a 5 to 10-membered cycloalkyl, cycloalkenyl, heterocycloalkyl, or heterocycloalkenyl, optionally substituted at any position with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell, and wherein further preferably said targeting fragment is capable of binding to a cell surface receptor. In a preferred embodiment, said R¹ is —H. In a preferred embodiment, said R¹ is —CH₃.

In a preferred embodiment of any aspects of the present disclosure and invention, said Ring A is cyclooctene, succinimide, or 7- to 8-membered heterocycloalkenyl, wherein the heterocycloalkyl or heterocycloalkenyl comprises one or two heteroatoms selected from N, O and S, and wherein each cyclooctene, heterocycloalkyl or heterocycloalkenyl is optionally substituted at any position with one or more R^{A1}, wherein preferably R^{A1} is oxo or fluorine, or wherein two R^{A1} combine to form one or more fused phenyl rings, preferably one or two fused phenyl rings, wherein each phenyl ring is optionally substituted with one or more —SO₃H or —OSO₃H.

In a preferred embodiment of any aspects of the present disclosure and invention, said conjugate of Formula I is a conjugate selected from:

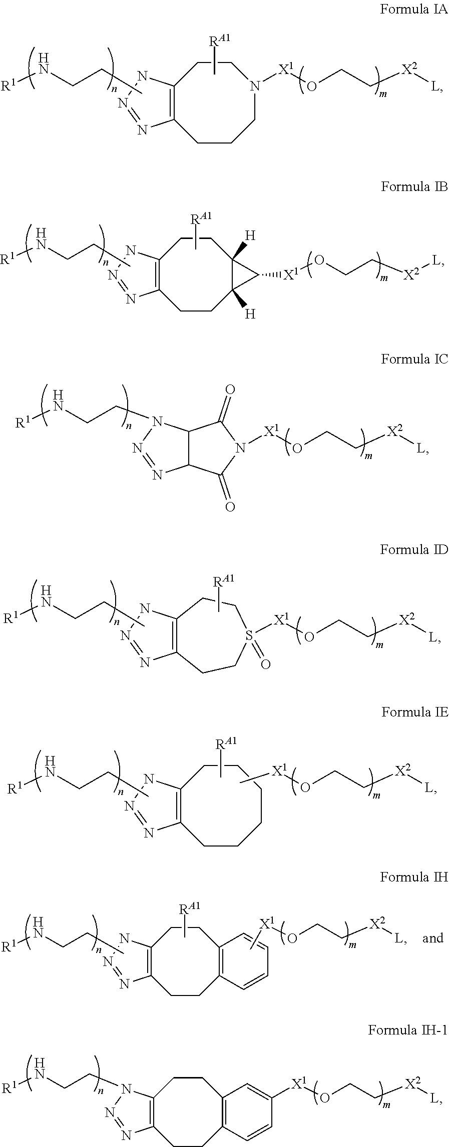

wherein R¹, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment said conjugate of Formula I is a conjugate selected from:

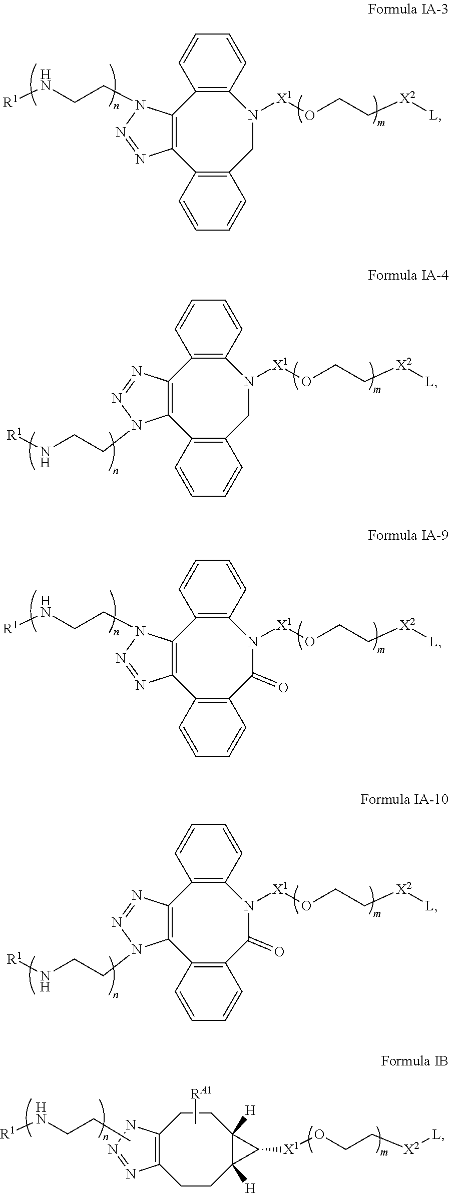

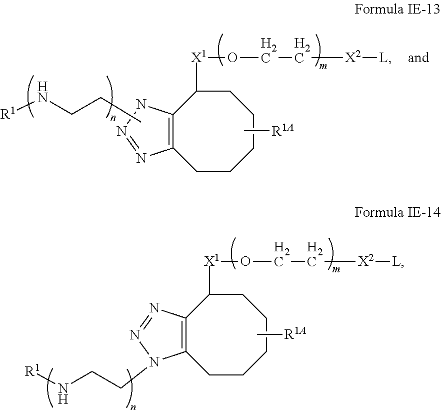

wherein R¹, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment of any aspects of the present disclosure and invention, said conjugate of Formula I is a conjugate selected from:

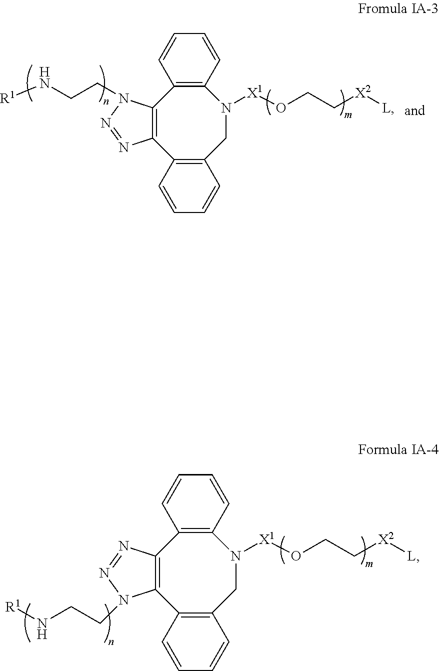

wherein R¹, X¹, X², L, m and n are as defined herein, preferably as defined in any embodiment described herein, be it individually related to each parameter R¹, X¹, X², L, m and n, or collectively to some or all of R¹, X¹, X², L, m and n.

In a preferred embodiment of any aspects of the present disclosure and invention, said conjugate of Formula I is a conjugate selected from:

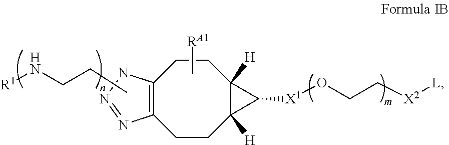

wherein R¹, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment of any aspects of the present disclosure and invention, said conjugate of Formula I is a conjugate selected from:

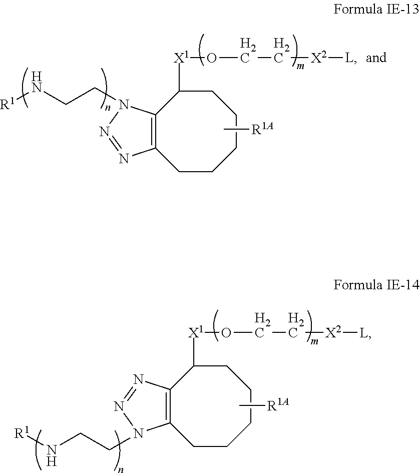

wherein R¹, R^{A1}, X¹, X², L, m and n are as defined herein, preferably as defined in any embodiment described herein, be it individually related to each parameter R¹, R^{A1}, X¹, X², L, m and n, or collectively to some or all of R¹, R^{A1}, X¹, X², L, m and n.

In a preferred embodiment, said targeting fragment comprises or preferably consists of the DUPA residue (HOOC—(CH₂)₂—CH(COOH)—NH—CO—NH—CH(COOH)—(CH₂)₂—CO—). In a further very preferred embodiment, said targeting fragment consists of the DUPA residue (HOOC(CH₂)₂—CH(COOH)—NH—CO—NH—CH(COOH)—(CH₂)₂—CO—), wherein both chiral C-atoms having (S)-configuration, as depicted in formula 1*.

In another preferred embodiment, said targeting fragment is epidermal growth factor (EGF), and wherein preferably said targeting fragment is human EGF (hEGF), and wherein again further preferably said targeting fragment comprises, preferably consists of, the sequence of SEQ ID NO:7.

### Synthesis and Characterization of Polyplexes

The present invention relates to polyplexes comprising a linear conjugate (e.g., a linear conjugate comprising LPEI, PEG, and a targeting fragment such as hEGF) polyplexed with a nucleic acid. As shown in the Examples, polyplexes can be prepared by incubating the inventive triconjugates together with nucleic acids such as in particular mRNAs and pDNAs. In some embodiments, polyplexes can form spontaneously (e.g., within an hour or within 30 minutes) by combining the inventive triconjugates with the nucleic acids in a solution of HEPES-buffered glucose at pH 7-7.4 (e.g., at room temperature), or in 5% glucose, or in HEPES buffered saline (HBS) pH 7.2, or in an acetate solution at pH 4-4.5 containing 5% glucose e.g., at room temperature).

The particle size distribution (reported as the z-average diameter and PDI) and ζ-potential of the polyplexes can be measured by dynamic light scattering (DLS) and electrophoretic mobility, respectively. DLS measures the light scatter intensity fluctuations of polyplexes caused by the Brownian motions and calculates hydrodynamic diameter (nm) using the Stokes-Einstein equation. Zeta potential (ζ-potential) measures the electrokinetic potential of the polyplexes.

In some embodiments, the z-average diameter and ζ-potential can be modified as a function of the N/P ratio, defined as the ratio of nitrogen atoms in LPEI to phosphorous atoms in nucleic acids. In some preferred embodiments, the z-average diameter of an inventive polyplex is below about 300 nm, more preferably below about 250 nm, yet more preferably below about 200 nm. Without wishing to be bound by theory, polyplexes with z-average diameters below about 200 nm are believed to be well-tolerated in vivo (e.g., exhibit high biodistribution and clearance) and are typically stable and not prone to aggregate formation.

In some preferred embodiments, the N/P ratio of the polyplexes is at least 2, at least 2.4, at least 2.5, at least 3, at least 3.5, is at least about 4, at least 4.5, at least 5, or at least 6. In some preferred embodiments, the N/P ratio is 2, 2.5, 3, 3.5, 4, 4.5, 5, 5.5, 6, 7, 8, 9, 10, 11 or 12. As shown herein, the N/P ratios mentioned above can provide polyplexes of acceptable size and stability for said polyplexes containing polyanions, such as and preferably nucleic acids.

In a preferred embodiment, said polyplexes of the invention have a mono- or bi-modal diameter distribution, preferably a monomodal diameter distribution. Preferably, said monomodal diameter distribution is within the sub-micrometer range.

In a preferred embodiment, said polyplexes have a z-average diameter of less than or equal to 350 nm. In a preferred embodiment, said polyplexes have a z-average diameter of less than or equal to about 300 nm. In another preferred embodiment, said polyplexes have a z-average diameter of less than or equal to 250 nm. In another preferred embodiment, said polyplexes have a z-average diameter of less than or equal to 210 nm. In another preferred embodiment, said polyplexes have a z-average diameter of less than or equal to 200 nm. In another preferred embodiment, said polyplexes have a z-average diameter of less than or equal to 180 nm. In another preferred embodiment, said polyplexes have a z-average diameter of less than or equal to 150 nm. In another preferred embodiment, said polyplexes have a z-average diameter of between 350 nm and 50 nm. In another preferred embodiment, said polyplexes have a z-average diameter of between 350 nm and 70 nm. In another preferred embodiment, said polyplexes have a z-average diameter of between 350 nm and 100 nm. In another preferred embodiment, said polyplexes have a z-average diameter of between 300 nm and 50 nm. In another preferred embodiment, said polyplexes have a z-average diameter of between 300 nm and 70 nm. In another preferred embodiment, said polyplexes have a z-average diameter of between 300 nm and 100 nm. In another more preferred embodiment, said polyplexes have a z-average diameter of between 250 nm and around 50 nm. In another more preferred embodiment, said polyplexes have a z-average diameter of between 250 nm and around 70 nm.

In another more preferred embodiment, said polyplexes have a z-average diameter of between 250 nm and around 100 nm. In another preferred embodiment, said polyplexes have a z-average diameter of between around 200 nm and around 50 nm. In another preferred embodiment, said polyplexes have a z-average diameter of between around 200 nm and around 70 nm. In another preferred embodiment, said polyplexes have a z-average diameter of between around 200 nm and around 100 nm. In another preferred embodiment, said polyplexes have a z-average diameter of between around 180 nm and around 50 nm. In another preferred embodiment, said polyplexes have a z-average diameter of between around 180 nm and around 70 nm. Preferably, said polyplexes have a mono-modal diameter distribution, preferably within the sub-micrometer range.

In a preferred embodiment, said polyplexes have a z-average diameter of less than or equal to 350 nm, and the N/P ratio of the polyplexes is at least 2, preferably at least 2.4. In a preferred embodiment, said polyplexes have a z-average diameter of less than or equal to about 300 nm, and the N/P ratio of the polyplexes is at least 2, preferably at least 2.4. In a preferred embodiment, said polyplexes have a z-average diameter of less than or equal to about 250 nm, and the N/P ratio of the polyplexes is at least 2, preferably at least 2.4. In a preferred embodiment, said polyplexes have a z-average diameter of less than or equal to about 220 nm, and the N/P ratio of the polyplexes is at least 2.4, more preferably at least 3, yet more preferably at least 4. In another preferred embodiment, said polyplexes have a z-average diameter of less than or equal to 200 nm, and the N/P ratio of the polyplexes is at least 3, preferably at least 4.

In another preferred embodiment, said polyplexes have a z-average diameter of less than or equal to 180 nm, and the N/P ratio of the polyplexes is at least 3, preferably at least 4. In another preferred embodiment, said polyplexes have a z-average diameter of less than or equal to 150 nm. In another preferred embodiment, said polyplexes have a z-average diameter of between 350 nm and 100 nm, and the N/P ratio of the polyplexes is at least 3, preferably at least 4. In another preferred embodiment, said polyplexes have a z-average diameter of between 300 nm and 100 nm, and the N/P ratio of the polyplexes is at least 3, preferably at least 4. In another more preferred embodiment, said polyplexes have a z-average diameter of between 250 nm and around 100 nm, and the N/P ratio of the polyplexes is at least 3, preferably at least 4. In another preferred embodiment, said polyplexes have a z-average diameter of between around 200 nm and around 100 nm, and the N/P ratio of the polyplexes is at least 3, preferably at least 4. Preferably, said polyplexes have a mono-modal diameter distribution, preferably within the sub-micrometer range.

In a preferred embodiment, the composition of the invention has a polydispersity index (PDI) of 0.7 or less. More preferably, said PDI is 0.5 or less, e.g. between 0.5 and 0.05. Again more preferably, said PDI is 0.35 or less, e.g. between 0.35 and 0.05. In another preferred embodiment, said PDI is 0.25 or less, e.g. between 0.25 and 0.05. In another preferred embodiment, said PDI is 0.2 or less, e.g. between 0.2 and 0.05. In another preferred embodiment said PDI is less than 0.2, e.g. between 0.19 and 0.05. In another more preferred embodiment said PDI is between 0.2 and 0.1. In another preferred embodiment said PDI is between 0.25 and 0.1. Preferably, said polyplexes have a mono-modal diameter distribution, preferably within the sub-micrometer range.

In a preferred embodiment, the composition of the invention has a polydispersity index (PDI) of 0.7 or less, and the N/P ratio of the polyplexes is at least 2, preferably at least 2.4. More preferably, said PDI is 0.5 or less, e.g. between 0.5 and 0.05. Again more preferably, said PDI is 0.35 or less, e.g. between 0.35 and 0.05, and the N/P ratio of the polyplexes is at least 2, preferably at least 2.4. In another preferred embodiment, said PDI is 0.25 or less, e.g. between 0.25 and 0.05, and the N/P ratio of the polyplexes is at least 2.4, more preferably at least 3, yet more preferably at least 4. In another preferred embodiment, said PDI is 0.2 or less, e.g. between 0.2 and 0.05, and the N/P ratio of the polyplexes is at least 3, preferably at least 4. In another preferred embodiment said PDI is less than 0.2, e.g. between 0.19 and 0.05, and the N/P ratio of the polyplexes is at least 3, preferably at least 4. In another more preferred embodiment said PDI is between 0.2 and 0.1. In another preferred embodiment said PDI is between 0.25 and 0.1, and the N/P ratio of the polyplexes is at least 3, preferably at least 4. Preferably, said polyplexes have a mono-modal diameter distribution, preferably within the sub-micrometer range.

In a preferred embodiment, said polyplexes have a z-average diameter of less than or equal to 350 nm, the PDI is 0.5 or less and the N/P ratio of the polyplexes is at least 2, preferably at least 2.4. In a preferred embodiment, said polyplexes have a z-average diameter of less than or equal to 350 nm, the PDI is 0.4 or less and the N/P ratio of the polyplexes is at least 2, preferably at least 2.4. In a preferred embodiment, said polyplexes have a z-average diameter of less than or equal to about 300 nm, the PDI is 0.4 and the N/P ratio of the polyplexes is at least 2, preferably at least 2.4. In another preferred embodiment, said polyplexes have a z-average diameter of less than or equal to about 250 nm, the PDI is 0.2 or less and the N/P ratio of the polyplexes is at least 2, preferably at least 2.4. In a preferred embodiment, said polyplexes have a z-average diameter of less than or equal to about 220 nm, the PDI is 0.2 or less, and the N/P ratio of the polyplexes is at least 2.4, more preferably at least 3, yet more preferably at least 4. In another preferred embodiment, said polyplexes have a z-average diameter of less than or equal to 200 nm, the PDI is 0.2 or less, and the N/P ratio of the polyplexes is at least 3, preferably at least 4. In another preferred embodiment, said polyplexes have a z-average diameter of less than or equal to 180 nm, the PDI is 0.2 or less, and the N/P ratio of the polyplexes is at least 3, preferably at least 4. In another preferred embodiment, said polyplexes have a z-average diameter of less than or equal to 150 nm, the PDI is 0.2 or less, and the N/P ratio of the polyplexes is at least 3, preferably at least 4. In another preferred embodiment, said polyplexes have a z-average diameter of between 350 nm and 100 nm, the PDI is 0.2 or less, and the N/P ratio of the polyplexes is at least 3, preferably at least 4. In another preferred embodiment, said polyplexes have a z-average diameter of between 300 nm and 100 nm, the PDI is 0.2 or less, and the N/P ratio of the polyplexes is at least 3, preferably at least 4. In another more preferred embodiment, said polyplexes have a z-average diameter of between 250 nm and around 100 nm, the PDI is 0.2 or less, and the N/P ratio of the polyplexes is at least 3, preferably at least 4. In another preferred embodiment, said polyplexes have a z-average diameter of between around 200 nm and around 100 nm, the PDI is 0.2 or less, and the N/P ratio of the polyplexes is at least 3, preferably at least 4. Preferably, said polyplexes have a mono-modal diameter distribution, preferably within the sub-micrometer range.

In a preferred embodiment, the composition of the invention has a zeta potential of greater than or equal to 18 mV, e.g. between 18 mV and 50 or 60 mV. In a preferred embodiment, the composition of the invention has a zeta potential of greater than or equal to 18 mV, e.g. between 18 mV and 60 mV. In a preferred embodiment, the composition of the invention has a zeta potential of greater than or equal to 18 mV, e.g. between 18 mV and 45 mV. In another preferred embodiment, the composition of the invention has a zeta potential of greater than or equal to 18 mV, e.g. between 18 mV and 42 mV. In another preferred embodiment, the composition of the invention has a zeta potential between 20 mV and 50 mV. In another preferred embodiment, the composition of the invention has a zeta potential between 20 mV and around 45 mV. In another preferred embodiment, the composition of the invention has a zeta potential between 20 mV and around 42 mV. In another preferred embodiment, the composition of the invention has a zeta potential between around 20 mV and around 40 mV. Preferably, said polyplexes have a mono-modal diameter distribution, preferably within the sub-micrometer range.

In a preferred embodiment, the composition of the invention has a zeta potential of greater than or equal to 18 mV, preferably between 18 mV and 50 mV, and the N/P ratio of the polyplexes is at least 2, preferably at least 2.4. In a more preferred embodiment, the composition of the invention has a zeta potential of greater than or equal to 18 mV, preferably between 18 mV and 45 mV, and the N/P ratio of the polyplexes is at least 2.4, more preferably at least 3, yet more preferably at least 4. In another preferred embodiment, the composition of the invention has a zeta potential of greater than or equal to 18 mV, e.g. between 18 mV and 42 mV, and the N/P ratio of the polyplexes is at least 3, preferably at least 4. In another preferred embodiment, the composition of the invention has a zeta potential between 20 mV and 50 mV, and the N/P ratio of the polyplexes is at least 3, preferably at least 4. In another preferred embodiment, the composition of the invention has a zeta potential between 30 mV and around 40 mV, and the N/P ratio of the polyplexes is at least 3, preferably at least 4. In another more preferred embodiment, the composition of the invention has a zeta potential between 18 mV and around 40 mV, and the N/P ratio of the polyplexes is at least 3, preferably at least 4. In another even more preferred embodiment, the composition of the invention has a zeta potential between around 20 mV and around 40 mV, and the N/P ratio of the polyplexes is at least 3, preferably at least 4. Preferably, said polyplexes have a mono-modal diameter distribution, preferably within the sub-micrometer range.

In a preferred embodiment, said polyplexes have a z-average diameter of less than or equal to 350 nm, and the N/P ratio of the polyplexes is at least 2, preferably at least 2.4, and the composition of the invention has a zeta potential of between 18 mV and 50 mV. In a preferred embodiment, said polyplexes have a z-average diameter of less than or equal to about 300 nm, and the N/P ratio of the polyplexes is at least 2, preferably at least 2.4, and the composition of the invention has a zeta potential of between 20 mV and 50 mV. In a preferred embodiment, said polyplexes have a z-average diameter of less than or equal to about 250 nm, and the N/P ratio of the polyplexes is at least 2, preferably at least 2.4, and the composition of the invention has a zeta potential of between 20 mV and 50 mV. In a preferred embodiment, said polyplexes have a z-average diameter of less than or equal to about 220 nm, and the N/P ratio of the polyplexes is at least 2.4, more preferably at least 3, yet more preferably at least 4, and the composition of the invention has a zeta potential of between 18 mV and 45 mV. In another preferred embodiment, said polyplexes have a z-average diameter of less than or equal to 200 nm, and the N/P ratio of the polyplexes is at least 3, preferably at least 4, and the composition of the invention has a zeta potential of between 18 mV and 45 mV. In another preferred embodiment, said polyplexes have a z-average diameter of less than or equal to 180 nm, and the N/P ratio of the polyplexes is at least 3, preferably at least 4, and the composition of the invention has a zeta potential of between 18 mV and 45 mV. In another preferred embodiment, said polyplexes have a z-average diameter of less than or equal to 150 nm, and the N/P ratio of the polyplexes is at least 3, preferably at least 4 and the composition of the invention has a zeta potential of between 18 mV and 45 mV. In another preferred embodiment, said polyplexes have a z-average diameter of between 350 nm and 100 nm, and the N/P ratio of the polyplexes is at least 3, preferably at least 4, and the composition of the invention has a zeta potential of between 18 mV and 45 mV. In another preferred embodiment, said polyplexes have a z-average diameter of between 300 nm and 100 nm, and the N/P ratio of the polyplexes is at least 3, preferably at least 4, and the composition of the invention has a zeta potential of between 18 mV and 45 mV. In another more preferred embodiment, said polyplexes have a z-average diameter of between 250 nm and around 100 nm, and the N/P ratio of the polyplexes is at least 3, preferably at least 4, and the composition of the invention has a zeta potential of between 18 mV and 45 mV. In another preferred embodiment, said polyplexes have a z-average diameter of between around 200 nm and around 100 nm, and the N/P ratio of the polyplexes is at least 3, preferably at least 4, and the composition of the invention has a zeta potential of between 18 mV and 45 mV. Preferably, said polyplexes have a mono-modal diameter distribution, preferably within the sub-micrometer range.

In a preferred embodiment, said polyplexes have a z-average diameter of less than or equal to 350 nm, the PDI is between 0.5 and 0.05, the N/P ratio of the polyplexes is at least 2, preferably at least 2.4, and the composition of the invention has a zeta potential of between 18 mV and 50 mV. In a preferred embodiment, said polyplexes have a z-average diameter of less than or equal to about 300 nm, the PDI is between 0.5 and 0.05, and the N/P ratio of the polyplexes is at least 2, preferably at least 2.4, and the composition of the invention has a zeta potential of between 18 mV and 50 mV. In a preferred embodiment, said polyplexes have a z-average diameter of less than or equal to about 250 nm, the PDI is between 0.35 and 0.05, the N/P ratio of the polyplexes is at least 2, preferably at least 2.4, and the composition of the invention has a zeta potential of between 18 mV and 50 mV. In a preferred embodiment, said polyplexes have a z-average diameter of less than or equal to about 220 nm, the PDI is 0.3 or less, e.g. between 0.3 and 0.05, the N/P ratio of the polyplexes is at least 2.4, more preferably at least 3, yet more preferably at least 4, and the composition of the invention has a zeta potential of between 18 mV and 45 mV. In another preferred embodiment, said polyplexes have a z-average diameter of less than or equal to 200 nm, the PDI is 0.2 or less, e.g. between 0.2 and 0.05, the N/P ratio of the polyplexes is at least 3, preferably at least 4, and the composition of the invention has a zeta potential of between 18 mV and 45 mV. In another preferred embodiment, said polyplexes have a z-average diameter of less than or equal to 180 nm, the PDI is 0.2 or less, e.g. between 0.2 and 0.05, and the N/P ratio of the polyplexes is at least 3, preferably at least 4, and the composition of the invention has a zeta potential of between 18 mV and 45 mV. In another preferred embodiment, said polyplexes have a z-average diameter of less than or equal to 150 nm, the PDI is 0.2 or less, e.g. between 0.2 and 0.05, the N/P ratio of the polyplexes is at least 3, preferably at least, and the composition of the invention has a zeta potential of between 18 mV and 45 mV. In another preferred embodiment, said polyplexes have a z-average diameter of between 350 nm and 100 nm, the PDI is 0.2 or less, e.g. between 0.2 and 0.05, the N/P ratio of the polyplexes is at least 3, preferably at least 4, and the composition of the invention has a zeta potential of between 18 mV and 45 mV. In another preferred embodiment, said polyplexes have a z-average diameter of between 300 nm and 100 nm, the PDI is 0.2 or less, e.g. between 0.2 and 0.05, the N/P ratio of the polyplexes is at least 3, preferably at least 4, and the composition of the invention has a zeta potential of between 25 mV and 45 mV. In another more preferred embodiment, said polyplexes have a z-average diameter of between 250 nm and around 100 nm, the PDI is 0.2 or less, e.g. between 0.2 and 0.05, the N/P ratio of the polyplexes is at least 3, preferably at least 4, and the composition of the invention has a zeta potential of between 18 mV and 45 mV. In another preferred embodiment, said polyplexes have a z-average diameter of between around 200 nm and around 100 nm, the PDI is 0.2 or less, e.g. between 0.2 and 0.05, the N/P ratio of the polyplexes is at least 3, preferably at least 4, and the composition of the invention has a zeta potential of between 18 mV and 45 mV. Preferably, said polyplexes have a mono-modal diameter distribution, preferably within the sub-micrometer range.

In some embodiments, the polyplex has a z-average diameter below about 200 nm. In some embodiments, the N/P ratio of the polyplex is between about 3 and about 10, preferably wherein the N/P ratio of the polyplex is between about 4 and about 7. In some embodiments, the N/P ratio of the polyplex is about 4, 5 or 7. In some preferred embodiments, the polyplexes of the present disclosure have a ζ-potential between about 15 and about 70 mV, between about 20 and about 70 mV; preferably between about 15 and about 50 mV; preferably between about 15 and about 40 mV.

### Polyplexes for Use in Treating Disease

In one aspect, the present invention provides compositions comprising polyplexes described herein for use in the treatment of a disease or disorder. In another aspect, the present invention provides the use of polyplexes described herein for use in the manufacture of a medicament for the treatment of a disease or disorder. In another aspect, the present invention provides a method of treating a disease or disorder in a subject in need thereof, the method comprising administering to the subject an effective amount of a polyplex as described herein.

In one aspect, the present invention provides compositions comprising polyplexes described herein for use in the treatment of disease or disorder such as cancer. In another aspect, the present invention provides the use of polyplexes described herein for use in the manufacture of a medicament for the treatment of a disease or disorder such as a cancer. In another aspect, the present invention provides a method of treating a disease or disorder such as a cancer in a subject in need thereof, the method comprising administering to the subject an effective amount of a polyplex as described herein.

In some embodiments, the cancer can be characterized by cells that express, highly express, or overexpress one or more cell surface receptors and/or antigens. Without wishing to be bound by theory, the triconjugates and/or polyplexes of the present invention can be targeted to a particular cell type (e.g., cancer cell type) by selecting an appropriate targeting fragment and coupling the appropriate targeting fragment to the PEG fragment to form a targeted triconjugate as described above. The cell surface receptor and/or antigen may be, but is not limited to, EGFR; HER2; an integrin (e.g., an RGD integrin); a sigma-2 receptor; Trop-2; folate receptor; prostate-specific membrane antigen (PSMA); p32 protein; a somatostatin receptor such as somatostatin receptor 2 (SSTR2); an insulin-like growth factor 1 receptor (IGF1R); a vascular endothelial growth factor receptor (VEGFR); a platelet-derived growth factor receptor (PDGFR); and/or a fibroblast growth factor receptor (FGFR).

In some embodiments, the cancer can be characterized by cells that have increased expression (e.g., overexpression or high expression) of EGFR. In some preferred embodiments, cancers characterized by cells that have increased expression of EGFR can be treated with polyplexes comprising an EGFR-targeting fragment such as hEGF. In certain embodiments, the cancer characterized by EGFR-overexpressing cells is an adenocarcinoma, squamous cell carcinoma, lung cancer (e.g., non-small-cell-lung-carcinoma), breast cancer, glioblastoma, head and neck cancer (e.g., head and neck squamous cell carcinoma), renal cancer, colorectal cancer, ovarian cancer, cervical cancer, bladder cancer or prostate cancer, and/or metastases thereof.

In certain embodiments, the cancer can be characterized by cells that have increased expression (e.g., overexpression or high expression) of HER2. In some preferred embodiments, cancers characterized by cells that have increased expression of HER2 can be treated with polyplexes comprising a HER2-targeting fragment such as anti-HER2 peptide (e.g., an anti-HER2 antibody or affibody). In some embodiments, the cancer characterized by HER2-overexpressing cells is breast cancer, ovarian cancer, stomach (gastric) cancer, and/or uterine cancer (e.g., aggressive forms of uterine cancer, such as uterine serous endometrial carcinoma) and/or metastases thereof In certain embodiments, the HER2 overexpressing cells are treatment-resistant cells (e.g., Herceptin/trastusumab resistant cells). Thus, the polyplex of the present invention may be for use in the treatment of Herceptin/trastusumab resistant cancer, i.e. cancer comprising cells that do not respond or respond to a lesser extent to exposure to Herceptin/trastusumab.

In some embodiments, the cancer can be characterized by cells that have increased expression (e.g., overexpression or high expression) of prostate-specific membrane antigen. In some preferred embodiments, cancers characterized by cells that have increased expression of prostate-specific membrane antigen (PSMA) can be treated with polyplexes comprising a PSMA-targeting fragment such as DUPA. In certain embodiments, the cancer characterized by PSMA-overexpressing cells is prostate cancer and/or metastases thereof. In a preferred embodiment, said cancer is prostate cancer.

In some embodiments, cancer-associated neovasculature can be characterized by increased expression (e.g., overexpression or high expression) of PSMA (see., e.g., Van de Wiele et al., Histol Histopathol., (2020); 35(9):919-927). In some preferred embodiments, cancers characterized by neovasculature that has increased expression of prostate-specific membrane antigen (PSMA) can be treated with polyplexes comprising a PSMA-targeting fragment such as DUPA. In some preferred embodiments, the cancers characterized by association with PSMA-overexpressing neovasculature are glioblastoma, breast cancer, bladder cancer and/or metastases thereof.

In some embodiments, the cancer can be characterized by cells that have increased expression (e.g., overexpression or high expression) of folate receptor. In some preferred embodiments, cancers characterized by cells that have increased expression of folate receptor can be treated with polyplexes comprising folate and/or folic acid as a targeting fragment. In certain embodiments, the cancer characterized by folate receptor-overexpressing cells is gynecological, breast, cervical, uterine, colorectal, renal, nasopharyngeal, ovarian, endometrial cancers and/or metastases thereof.

In some embodiments, the cancer can be characterized by cells that have increased expression (e.g., overexpression or high expression) of somatostatin receptors such as somatostatin receptor 2 (SSTR2). In some embodiments, cancers characterized by increased expression of SSTR2 can be treated with polyplexes comprising a somatostatin receptor-targeting fragment such as somatostatin and/or octreotide. In certain embodiments, cancers characterized by increased expression of somatostatin receptors (e.g., SSTR2) include colorectal cancer and/or metastases thereof.

In some embodiments, the cancer can be characterized by cells that have increased expression of integrins (e.g., RGD integrins such as αᵥβ₆ integrin or αᵥβ₈ integrin). In some embodiments, cancers characterized by increased expression of integrins such as RGD integrins can be treated with polyplexes comprising an integrin-targeting fragment such as arginine-glycine-aspartic acid (RGD)-containing ligands (e.g., cyclic RGD ligands). In some preferred embodiments, the integrin-targeting fragment can be a peptide such as SFITGv6, SFFN1, SFTNC, SFVTN, SFLAP1, SFLAP3, A20FMDV2 (see, e.g., Roesch et al., J. Nucl. Med. 2018, 59 (11) 1679-1685). In some embodiments, the integrin-targeting fragment can be an anti-integrin antibodies such as anti αᵥβ₆ integrin antibodies, anti-integrin diabodies, or knottins. In some embodiments, the integrin-targeting fragment can be latent transforming growth factor-B (TGFß). In some embodiments, cancer cells characterized by increased expression of integrins such as RGD integrins can include solid tumor, breast cancer, ovarian cancer, cervical cancer, pancreatic cancer, non-small cell lung cancer (NSCLC), colon cancer, oral squamous cell cancer, astrocytoma, head and neck squamous cell carcinoma and/or metastases thereof.

In some embodiments, the cancer can be characterized by cells that exist in a low pH microenvironment. In some embodiments, cancers characterized by a low pH microenvironment can be treated with polyplexes comprising low pH insertion peptides (pHLIPs) as a targeting fragment. In some preferred embodiments, cancers characterized by cells exist in a low pH microenvironment include breast cancer and/or metastases thereof.

In some embodiments, the cancer can be characterized by cells that have increased expression of asialoglycoprotein receptors. In some embodiments, cancers characterized by increased expression of asialoglycoprotein receptors can be treated with polyplexes comprising an asialoglycoprotein receptor-targeting fragment such as asialoorosomucoid. In certain embodiments, the cancer characterized by increased expression of asialoglycoprotein receptors is liver cancer, gallbladder cancer, stomach cancer and/or metastases thereof.

In some embodiments, the cancer can be characterized by cells that have increased expression of insulin receptors. In some embodiments, cancers characterized by increased expression of insulin receptors can be treated with polyplexes comprising an insulin-receptor targeting fragment such as insulin. In certain embodiments, the cancer characterized by insulin-receptor overexpressing cells is breast cancer, prostate cancer, endometrial cancer, ovarian cancer, liver cancer, bladder cancer, lung cancer, colon cancer, thyroid cancer and/or metastases thereof.

In some embodiments, the cancer can be characterized by cells that have increased expression of mannose-6-phosphate receptors (e.g., monocytes). In some embodiments, cancers characterized by increased expression of mannose-6-phosphate receptors can be treated with polyplexes comprising a mannose-6-phosphate receptor targeting fragment such as mannose-6-phosphate. In some embodiments, the cancer characterized by overexpression of mannose-6-phosphate receptor is leukemia.

In some embodiments, the cancer can be characterized by cells that have increased expression of mannose receptors. In some embodiments, cancers characterized by increased expression of mannose receptors can be treated with polyplexes comprising a mannose-receptor targeting fragment such as mannose. In some embodiments, cancers characterized by increased expression of mannose receptors include gastric cancer and/or metastases thereof.

In some embodiments, the cancer can be characterized by cells that have increased expression of glycosides such as Sialyl Lewis^{x} antigens. In some embodiments, cancers characterized by increased expression of Sialyl Lewis^{x} antigens can be treated with polyplexes comprising Sialyl Lewis^{x} antigen targeting fragments such as E-selectin.

In some embodiments, the cancer can be characterized by cells that have increased expression of N-acetyllactosamine. In some embodiments, cancers characterized by increased expression of N-acetyllactosamine can be treated with polyplexes comprising an N-acetyllactosamine targeting fragment.

In some embodiments, the cancer can be characterized by cells that have increased expression of galactose. In some embodiments, cancers characterized by increased expression of galactose can be treated with polyplexes comprising a galactose targeting fragment. In some embodiments, cancers characterized by increased expression of galactose include colon carcinoma and/or metastases thereof.

In some embodiments, the cancer can be characterized by cells that have increased expression of sigma-2 receptors. In some embodiments, cancers characterized by increased expression of sigma-2 receptors can be treated with polyplexes comprising sigma-2 receptor agonists, such as N,N-dimethyltryptamine (DMT), sphingolipid-derived amines, and/or steroids (e.g., progesterone). In some embodiments, cancers characterized by increased expression of sigma-2 receptors include pancreatic cancer, lung cancer, breast cancer, melanoma, prostate cancer, ovarian cancer and/or metastases thereof.

In some embodiments, the cancer can be characterized by cells that have increased expression of the mitochondrial protein p32. In some embodiments, cancers characterized by increased expression of p32 can be treated with polyplexes comprising p32-targeting ligands 15 such as anti-p32 antibody or p32-binding LyP-1 tumor-homing peptide. In some embodiments, cancers characterized by increased expression of p32 include glioma, breast cancer, melanoma, endometrioid carcinoma, adenocarcinoma, colon cancer and/or metastases thereof.

In some embodiments, the cancer can be characterized by cells that have increased expression of Trop-2. In some embodiments, cancers characterized by increased expression of Trop-2 can be treated with polyplexes comprising a Trop-2 targeting fragment such as an anti-Trop-2 antibody and/or antibody fragment. In some embodiments, cancers characterized by increased expression of Trop-2 include breast cancer, squamous cell carcinoma, esophageal squamous cell carcinoma (SCC), pancreatic cancer, hilar cholangiocarcinoma, colorectal cancer, bladder cancer, cervical cancer, ovarian cancer, thyroid cancer, non-small-cell lung cancer (NSCLC), hepatocellular cancer, small cell lung cancer, prostate cancer, head and neck cancer, renal cell cancer, endometrial cancer, glioblastoma, gastric cancer and/or metastases thereof.

In some embodiments, the cancer can be characterized by cells that have increased expression (e.g., overexpression or high expression) of insulin-like growth factor 1 receptor. In some preferred embodiments, cancers characterized by cells that have increased expression of insulin-like growth factor 1 receptor can be treated with polyplexes comprising an insulin-like growth factor 1 receptor-targeting fragment, such as insulin-like growth factor 1. In some embodiments, the cancer characterized by insulin-like growth factor 1 receptor overexpressing cells is breast cancer, prostate cancer, lung cancer and/or metastases thereof.

In some embodiments, the cancer can be characterized by cells that have increased expression (e.g., overexpression or high expression) of VEGF receptor. In some preferred embodiments, cancers characterized by cells that have increased expression of VEGF receptor can be treated with polyplexes comprising a VEGF receptor-targeting fragment such as VEGF.

In some embodiments, the cancer can be characterized by cells that have increased expression (e.g., overexpression) of platelet-derived growth factor receptor. In some preferred embodiments, cancers characterized by cells that have increased expression of platelet-derived growth factor receptor can be treated with polyplexes comprising an platelet-derived growth factor receptor-targeting fragment such as platelet-derived growth factor. In some preferred embodiments, cancers characterized by cells that have increased expression of platelet-derived growth factor receptor include breast cancer and/or metastases thereof.

In some embodiments, the cancer can be characterized by cells that have increased expression (e.g., overexpression or high expression) of fibroblast growth factor receptor. In some preferred embodiments, cancers characterized by cells that have increased expression of fibroblast growth factor receptor can be treated with polyplexes comprising a fibroblast growth factor receptor-targeting fragment such as fibroblast growth factor.

In a further aspect, the present invention provides for the use of pharmaceutical compositions as described herein and comprising the inventive polyplexes which polyplexes comprises said pharmaceutically active nucleic acid encoding a pharmaceutically active peptide or protein for the therapeutic or prophylactic treatment of various diseases, in particular diseases in which provision of said peptide or protein to a subject results in a therapeutic or prophylactic effect. For example, provision of an antigen or epitope which is derived from a virus may be useful in the treatment of a viral disease caused by said virus. Provision of a tumor antigen or epitope may be useful in the treatment of a cancer disease wherein cancer cells express said tumor antigen. Provision of a cytokine or a cytokine-fusion may be useful to modulate tumor microenvironment. Provision of cytokines, hormones or growth factors can be used for the treatment of non-oncology related diseases.

### EQUIVALENTS

While the present invention has been described in conjunction with the specific embodiments set forth above, many alternatives, modifications and other variations thereof will be apparent to those of ordinary skill in the art. All such alternatives, modifications, and variations are intended to fall within the scope and spirit of the present invention.

### EXAMPLES

The invention is further illustrated by the following examples and synthesis schemes, which are not to be construed as limiting this invention in scope or spirit to the specific procedures herein described. It is to be understood that the examples are provided to illustrate certain embodiments and that no limitation to the scope of the invention is intended thereby. It is to be further understood that resort may be had to various other embodiments, modifications, and equivalents thereof which may suggest themselves to those skilled in the art without departing from the spirit of the present invention and/or scope of the appended claims.

Abbreviations used in the following examples and elsewhere herein are:

| | |
|---|---|
| ACN | Acetonitrile |
| Aoc | 8-aminooctanoic acid |
| Aq. | aqueous |
| ASGPr | Asialoglycoprotein Receptor |
| BCN | Bicyclononyne |
| D | Dispersity |
| DBCO | Dibenzocyclooctyne |
| DCM | Dichloromethane |
| DIEA | N,N-Diisopropylethylamine (Hünig's Base) |
| DLS | Dynamic light scattering |
| DMSO | Dimethyl sulfoxide |
| DTT | Dithiothreitol (reducing agent) |
| DUPA | 2-[3-(1,3-dicarboxypropyl)ureido]pentanedioic acid |
| EGF | Epidermal growth factor |
| ELSD | Evaporative light scattering detector |
| Endo-BCN | (1alpha,8alpha,9beta)-bicyclo[6.1.0]non-4-yne |
| Epsilon (ε) | Extinction coefficient |
| Eq | Equivalent |
| FLuc | Firefly Luciferase |
| FLuc mRNA | Firefly Luc messenger RNA |
| HATU | O-(7-Azabenzotriazol-1-yl)-N,N,N′,N′-tetramethyluronium- |
| | hexafluorphosphate |
| HBG | HEPES buffered glucose solution |
| hEGF | Human epidermal growth factor |
| HEPES | 4-(2-hydroxyethyl)-1-piperazineethanesulfonic acid |
| LC | Liquid chromatography |
| LPEI | Linear polyethyleneimine |
| LPEI-l-PEG-hEGF | Linear polyethyleneimine-PEG-human epidermal growth factor conjugate |
| MADOPA | N¹⁰-Methyl-4-amino-4-deoxypteroic acid |
| MAL | Maleimide |
| MCC | 4-(N-maleimidomethyl)-cyclohexane-1-carboxy linker |
| NHS | N-hydroxysuccinimide |
| OPSS | Orthopyridyl disulfide |
| PEG | Polyethylene glycol |
| PDI | Polydispersity Index |
| Poly(Glu) | Poly-L-glutamic acid sodium salt |
| PyBOP | Benzotriazol-1-yloxytripyrrolidinophosphonium hexafluorophosphate |
| RENCA | Renal Carcinoma |
| RP-HPLC | Reversed phase high pressure liquid chromatography |
| RP-HPLC-MS | Reversed phase high pressure liquid chromatography mass spectrometry |
| qTOF MS | Quadrupole time of flight mass spectrometry |
| SPDP | (succinimidyl 3-(2-pyridyldithio)propionate) |
| SSTR2 | Somatostatin receptor 2 |
| TCEP | Tris(2-carboxyethyl)phosphine |
| TFA | Trifluoroacetic acid |
| TFF | Tangential flow filtration |
| TIS | Triisopropylsilane |

Unless otherwise noted, the following polymer naming conventions are used herein. Linear (i.e., unbranched) polymers are denoted with “T” and random (i.e., branched) polymers are denoted with “r”. Conjugates are further identified using an abbreviation for each fragment of the conjugate (e.g., PEG or LPEI) and/or targeting group (e.g., hEGF) in the orientation in which they are connected. Subscripts, when used, after each fragment within the conjugate indicate the number of monomer units (e.g., LPEI or PEG units) in each fragment. The linking moieties, and in particular the divalent covalent linking moiety Z of Formula I* connecting the LPEI and PEG fragments (e.g., a 1, 2, 3 triazole or a 4,5-dihydro-1H-[1,2,3]triazole) are defined by the reactive groups that formed the linking moieties and the divalent covalent linking moiety Z of Formula I*, respectively. For example, the conjugate abbreviated “LPEI-l-[N₃:DBCO]-PEG₃₆-hEGF” is an unbranched (i.e., linear) conjugate comprising LPEI connected to a 36-unit PEG chain through a 1, 2, 3 triazole formed by the reaction of an azide comprised by the LPEI fragment and DBCO comprised by the PEG fragment, while the terminal end of the PEG fragment is bonded to hEGF.

Analytical Methods, Materials, and Instrumentation. Unless otherwise noted, reagents and solvents were used as received from commercial suppliers. Starting materials are either commercially available or made by known procedures in the reported literature or as illustrated. α-Hydrogen-ω-azido-poly(iminoethylene) (H—(NC₂H₅)ₙ—N₃; LPEI-N₃) ULTROXA® (MW=22 KDa; dispersity≤1.25) and α-Methyl-ω-azido-poly(iminoethylene) (CH₃—(NC₂H₅)ₙ—N₃; Me-LPEI-N₃) ULTROXA® (MW=25.3 KDa; dispersity≤1.25) were obtained from AVROXA BV (Belgium). DBCO-amine (Compound 35) was purchased from BROADPHARM Inc (USA) (Product No. BP-22066; C₁₈H₁₆N₂O; Mw 276.3), NHS-PEG₃₆-OPSS was purchased from Quanta Biodesign Ltd, (USA) (Product No. 10867; Mw 1969.3). DBCO-PEG₄-TFP (Product No. PEG6740, C₃₇H₃₈F₄N₂O₈; Mw 714.7), DBCO-PEG₁₂-TFP (Product No. JSI-A1201-068, C₅₃H₇₀F₄N₂O₈; Mw 1067.12), DBCO-PEG₂₄-TFP (ProductNo. PEG6760, C₇₇H₁₁₈F₄N₂O₂₈; Mw 1595.75), DBCO-PEG₂₄-MAL (Product No. JSI-A2405-004, C₇₆H₁₂₂N₄O₂₉; Mw 1555.79), CliCr®-beta-Ala-NH₂ (Product No. RL-4190), HOOC-dPEG₃₆-NH₂ (Product No. PEG3340, CAS No. 196936-04-6) all from IRIS BIOTECH GMBH (Germany). Poly(Glu) (MW range: 50-100 KDa) was obtained from Sigma Aldrich. DUPA-Aoc-Phe-Gly-Trp-Trp-Gly-Cys ((C₅₇H₇₁N₁₁O₁₆S; Mw 1198.3; SEQ ID NO:4), DUPA-Aoc-Phe-Gly-Trp-Trp-Gly-Maleimide (C₆₀H₇₂N₁₂O₁₆; Mw 1217.3; SEQ ID NO:5, hEGF peptides, and MCC-hEGF (C₂₈₂H₄₀₉N₇₉O₈₆S₇; Mw 6435) were synthesized by CBL Patras S.A. (Greece). Cys-GE-11 peptide (sequence: Cys-Tyr-His-Trp-Tyr-Gly-Tyr-Thr-Pro-Gln-Asn-Val-Ile; CYHWYGYTPQNVI, SEQ ID NO:6) was custom synthesized by GenScript Biotech (Netherlands) B.V. HER2 affibody was purchased from Abcam (Anti-ErbB2/HER2 Affibody® Molecule, Product No. ab31889). Folic acid (Product No. F7876) and N¹⁰-methyl-4-amino-4-deoxypteroic acid (Product No. 861553) were purchased from Sigma-Aldrich. Cysteamine 4-methoxytrityl resin (Novabiochem®; Product No. 8.56087.0001) was purchased from Merck KGaA. SCO-PEG₃-NH₂ (Product No. SC-8301) was purchased from Sichem GMBH. Tris-GalNAc₃-Ala-PEG₃-NH₂ (C₇₃H₃₂N₁₂O₃₂; Mw 1689.9) was purchased from Sussex Research Laboratories Inc. (Canada) (Product No. MV100017). Cell lines were obtained from ATCC®: A431 (No. CRL-1555); MCF7 (No. HTB-22); LNCaP (No. CRL-1740); PC-3 (No. CRL-1435); B16F10 cells and Renca parental cells (mouse renal carcinoma, no human EGFR). RencaEGFR M1 H cells (derivate of Renca parental engineered to overexpress human EGFR) were obtained from Georg-Speyer-Haus, Institute for Tumor Biology and Experimental Therapy, Frankfurt am Main, Germany. Acetate buffer was 50 mM sodium acetate (aq.) supplemented with 5% glucose at pH 4-4.5. HEPES buffer was HEPES at a concentration of 20 mM (aq.) at a pH of 7-7.4. Lipofectamine messenger MAX was purchased from ThermoFisher, and jetPEI was purchased from Polyplus (Cat #101000053). Cell culture reagents were purchased from Biological Industries, Bet Ha'emek, Israel. All reagents were used according to manufacturer's instructions at the indicated concentrations. Firefly Luciferase (Fluc) mRNA was purchased from TriLink Biotechnologies USA (cat #L-7602; 1.0 mg/mL in 1 mM Sodium Citrate, pH 6.4; mRNA Length: 1929 nucleotides). mRNA were purchased from TriLink Biotechnologies, USA or Tebubio GmbH, Germany: Luc mRNA (Trilink Biotechnologies, L-7602) comprising SEQ ID NO:15 (mRNA Luc ORF); Capped (CleanCap AG, TriLink) and 5′UTR, 3′UTR, poly A optimized for optimal translational efficiency. Renilla Luciferase mRNA (Trilink Biotechnologies, L-7204) comprising SEQ ID NO:16 (mRNA Renilla Luc ORF); Capped (CleanCap AG, TriLink); Full Substitution of Pseudo-U; Polyadenylated (120A); Human IL-2 mRNA (Trilink Biotechnologies, WOTL83314) comprising (SEQ ID NO:17 (mRNA hIL-2 ORF); Capped (CleanCap AG, TriLink); Fully substituted with Pseudo U; 5′UTR, 3′UTR, poly A optimized for optimal translational efficiency. Human IFNβ mRNA (Tebubio, TTAP-122022) comprising (SEQ ID NO:18 (mRNA hIFNP-2 ORF); Capped (Enzymatic capping with same performance as CleanCap AG, Tebubio); Fully substituted with Nlmethylspeudo U; 5′UTR, 3′UTR, poly A optimized for optimal translational efficiency. hIFNγ mRNA (Trilink Biotechnologies, WOTL87247 comprising SEQ ID NO:19 (mRNA hIFNγ ORF); Capped (CleanCap AG, TriLink); Fully substituted with Pseudo U; 5′UTR, 3′UTR, poly A optimized for optimal translational efficiency. EPO mRNA (Trilink Biotechnologies, L-7209) comprising SEQ ID NO:20 (mRNA EPO ORF); Capped (CleanCap AG, TriLink); Full Substitution of Pseudo-U; Polyadenylated (120A). Diphtheria toxin (DT) catalytic domain A (DT-A) mRNA (Tebubio, TTAP-012023 comprising SEQ ID NO:21 (mRNA DT-A ORF); Capped (Enzymatic capping with same performance as CleanCap AG, Tebubio); Fully substituted with Nlmethylspeudo U; 5′UTR, 3′UTR, poly A optimized for optimal translational efficiency. The following plasmid DNA were used: pGreenFire1-CMV Plasmid (SBI, Cat #TRO11PA-1); plasmid SZL (Invivogen, pSELECT-zeo-LucSh); plasmid hTL-2 (InvivoGen, Cat #pUNO1-hILO2); plasmid hIFND (Sino Biological, pCMV3-hIFNP).

UV spectrophotometry of samples comprising hEGF. Measurements of hEGF content in reagent solutions and in conjugated samples were performed on a microplate reader (Spectramax Paradigm, Molecular Devices) using Brand® pureGrade UV-transparent microplates at 280 nm. UV absorption of a 100 mL solution of sample in its buffer was measured and the absorbance of the sample was corrected by subtracting the absorbance of buffer solution alone (blank). F (280 nm) of hEGF was calculated with the following formula:

$${ε}_{( 280 )} = ( \# Trp ) * ( 5500 ) + ( \# Tyr ) * ( 1490 ) + ( \# cystine ) * ( 125 ) = 2 * ( 5500 ) + 5 * ( 1490 ) + 2 * ( 125 ) = {18}^{′} 700 {cm}^{- 1} \cdot {M}^{- 1} .$$

The concentration of total hEGF was calculated using the formula:

$$c ( hEGF ) [ mol / L ] = {A}_{280} [ AU ] / ( {ε}_{280} [ L * {mol}^{- 1} * {cm}^{- 1} ] * 0.28 cm ) .$$

UV spectrophotometry of samples comprising HER2. For measurements of HER2 (e.g., DBCO-PEG₂₄-HER2 or LPEI-PEG₂₄-HER2 content in samples), UV spectrophotometry was performed on a Thermofischer Nanodrop One C device at 280 nm. 2 mL of the sample were analysed and the absorbance of the sample was corrected for by subtracting the absorbance of 2 mL of the appropriate buffer solution alone (blank). F (280 nm) of HER2 was 16600 cm⁻¹-M⁻¹. The concentration of total HER2 was calculated using this formula:

$$c ( HER 2 ) [ mol / L ] = {A}_{280} [ AU ] / ( {ε}_{280} [ L * {mol}^{- 1} * {cm}^{- 1} ] * 1 cm ) .$$

UV spectrophotometry of samples comprising DUPA. For measurements of DUPA content, UV spectrophotometry was performed on a microplate reader (Spectramax Paradigm, Molecular Devices) at 280 nm. 100 μL of solution were analysed in Brand puregrade 98 UVtransp F as well as 100 μL of the appropriate buffer (blank). The absorbance of the sample was corrected for the blank. F (280 nm) of DUPA was (theoretically determined): F (280 nm)=11′000 cm⁻¹-M⁻¹. The concentration of DUPA was calculated using this formula: c(DUPA) [mol/L]=A280 [AU]/ε(F [L*mol⁻¹*cm⁻¹]*0.28 cm).

UV spectrophotometry of samples comprising DBCO. Measurements of DBCO content of reagent solution and conjugated samples were performed on a microplate reader (Spectramax Paradigm, Molecular Devices) using Brand® pureGrade UV-transparent microplates at 309 nm. UV absorption of a 100 mL buffered solution was measured and the absorbance of the sample was corrected by subtracting the absorbance of buffer solution alone (blank). F (309 nm) of DBCO was 12,000 cm⁻¹-M⁻¹. The concentration of total DBCO was calculated using this formula:

$$c ( DBCO ) [ mol / L ] = {A}_{309} [ AU ] / ( {ε}_{309} [ L * {mol}^{- 1} * {cm}^{- 1} ] * 0.28 cm ) .$$

RP-HPLC-coupled Mass Spectrometry. Samples were analyzed by LC-MS using an Agilent 1260 Infinity II HPLC system or an Agilent UHPLC 1290 system.

The Agilent 1260 Infinity II HPLC system was connected to an Agilent iFunnel 6550B qTOF equipped with an Agilent Jet Stream electrospray ionization (AJS ESI) source. The sample was separated on a Phenomenex Aeris Widepore column XB-C8-3.6 μm, 100×2.1 mm (P/N: OOD-4481-AN) at 40° C. 1-5 μL were injected and elution was achieved with the eluent gradient shown in Table 1 with a flowrate of 0.3 mL/min, where solvent A was 100% H₂O with 0.1% HCOOH and solvent B 100% ACN with 0.1% HCOOH. The AJS ESI source was operated with a capillary voltage of 3000 V and a nozzle voltage of 1000 V with a drying gas temperature of 200° C. and a flow rate of 14 μL/min, nebulizing gas pressure of 20 psig, and a sheath gas temperature of 325° C. and flow rate of 12 μL/min. MS data were acquired in the positive ion mode in the range of 100-3200 m/z in the standard mass range at 4 Ghz high resolution mode between 2 and 12 min. The fragmentor and octupole RF voltages were set at 380, 750 V respectively.

TABLE 1
Eluent Gradient for RP-HPLC-MS using
Agilent 1260 Infinity II HPLC System

| Time [min] | A [%] | B [%] |
|---|---|---|
| 0.00 | 95.00 | 5.00 |
| 1.00 | 95.00 | 5.00 |
| 8.00 | 50.00 | 50.00 |
| 9.00 | 5.00 | 95.00 |
| 13.00 | 5.00 | 95.00 |

The Agilent UHPLC 1290 system comprised an Agilent 1290 binary pump (G4220A), Agilent 1290 HiP Sampler (G4226A), Agilent 1290 Column compartment (G1316C), Agilent 1290 DAD UV modules (G4212A), and Agilent Quadrupole LC/MS (6130) at 40° C. using a Phenomenex BioZen column XB-C8 (3.6 μm, 150×2.1 mm (OOF-4766-AN) equipped with a 25 pre-column filter of the same material (AJO-9812). 5 μL of sample were injected. The flow was 0.4 mL/min. Signal was monitored at 210 nm, 215 nm, 240 nm and 280 nm. The mobile phases were: A) H₂O with 0.1% (vol.) HCOOH and B) ACN. The eluent gradient used is given in Table 2.

TABLE 2
Eluent Gradient for RP-HPLC-MS
using Agilent UHPLC 1290 System

| Time [min] | A [%] | B [%] |
|---|---|---|
| 0.00 | 95.00 | 5.00 |
| 1.00 | 95.00 | 5.00 |
| 8.00 | 50.00 | 50.00 |
| 9.00 | 5.00 | 95.00 |
| 11.00 | 5.00 | 95.00 |

Analytical RP-HPLC. RP-HPLC experiments were performed on an Agilent UHPLC 1290 system comprising an Agilent 1290 binary pump (G4220A), Agilent 1290 HiP Sampler (G4226A), Agilent 1290 Column Compartment (G1316C), and Agilent 1290 DAD UV (G4212A) modules at 40° C. using a Phenomenex BioZen™ XB-C8 column (3.6 μm, 150×2.1 mm (OOF-4766-AN) equipped with a pre-column filter of the same material (AJO-9812). 20 μL of sample were injected. The flow was 0.4 mL/min. Signal was monitored at 210 nm, 214 nm, 220 nm, 230 nm, 240 nm and 280 nm. The mobile phases were A) H₂O+0.1% TFA (vol.) and B) ACN+0.1% TFA (vol.). The eluent gradient used is given in Table 3.

TABLE 3
Eluent Gradient for Analytical RP-HPLC

| Time [min] | A [%] | B [%] |
|---|---|---|
| 0.00 | 95.00 | 5.00 |
| 1.00 | 95.00 | 5.00 |
| 8.00 | 50.00 | 50.00 |
| 9.00 | 5.00 | 95.00 |
| 11.00 | 5.00 | 95.00 |

Preparative RP-HPLC. Preparative RP-HPLC experiments were performed on a Waters preparative system or a PuriFlash RP preparative system.

The Waters system comprised a Waters 515 HPLC Pump, Waters 2545 Binary Gradient Module, Waters 2777C Sampler, Waters Fraction Collector III and Waters 2487 Dual λ Absorbance Detector module using a Phenomenex Kinetex 5 mm XB-C18 column (100 Å, 100×21.0 mm, OOD-4605-PO-AX) equipped with a Phenomenex SecurityGuard PREP Cartridge Core-shell C18 pre-column (15×21.2 mm, G16-007037). The flow rate was 35 mL/min and the signal was monitored at 240 nm. The fractions collector collected from 0.1 min to 30 min volumes of ˜8 mL/tube (88% total filling) according to the following profile: Eluent A: H₂O with 0.1% (vol.) TFA. Eluent B: CAN with 0.1% (vol) TFA. The eluent gradient used is given in Table 4.

TABLE 4
Eluent Gradient for Preparative RP-
HPLC Using Waters Preparative System

| Time [min] | A [%] | B [%] |
|---|---|---|
| 0.00 | 90.00 | 10.00 |
| 30.00 | 50.00 | 50.00 |
| 35.00 | 2.00 | 98.00 |
| 36.00 | 2.00 | 98.00 |
| 38.00 | 90.00 | 10.00 |

The PuriFlash system comprised an Interchim Inc. PuriFlash 1 Serie system comprising an injector, pump, detector and fraction collector using a Phenomenex Kinetex 5 mm XB-C18 column (100 Å, 100×21.0 mm, 00D-4605-PO-AX) equipped with a Phenomenex SecurityGuard PREP Cartridge Core-shell pre-column (C18 15×21.2 mm, G16-007037). When injecting (from 00 s to 04 s), the flow rate was 10 mL/min and then was 35 mL/min until the end of run. The signal was monitored at 210 nm. The mobile phases were: Eluent A: H₂O with 0.1% (vol) TFA. Eluent B: ACN with 0.1% (vol.) TFA. The eluent gradient used is given in Table 5.

TABLE 5
Eluent Gradient for Preparative RP-HPLC
Using PuriFlash Preparative System

| No | Time | Flow [mL/min] | A [%] | B [%] |
|---|---|---|---|---|
| 01 | 00 s | 10.0 | 90 | 10 |
| 02 | 01 s | 10.0 | 90 | 10 |
| 03 | 04 s | 10.0 | 90 | 10 |
| 04 | 01:03 | 10.0 | 89 | 11 |
| 05 | 01:06 | 10.0 | 89 | 11 |
| 06 | 01:35 | 10.0 | 88 | 12 |
| 07 | 01:38 | 35.0 | 88 | 12 |
| 08 | 30:00 | 35.0 | 50 | 50 |
| 09 | 35:00 | 35.0 | 02 | 98 |
| 10 | 36:00 | 35.0 | 02 | 98 |
| 11 | 38:00 | 35.0 | 90 | 10 |
| 12 | 40:00 | 35.0 | 90 | 10 |

Copper Assay. The copper assay provides the concentration in mg/mL of total LPEI present in the solution (Ungaro et al., J Pharm. Biomed. Anal. 31; 143-9 (2003)). A stock solution of copper reagent (10×) was prepared by dissolving 23.0 mg of CuSO₄·5H₂O in 10.0 mL acetate buffer (100 mM; pH 5.4). This stock solution was stored at 4° C. Prior to analysis, this reagent was diluted ten-fold with acetate buffer (100 mM pH 5.4) and used directly. As a control, a solution of known concentration of LPEI (in vivo-jetPEI; 150 mM nitrogen concentration; Polyplus 201-50G) was used. 6.7 μL aliquots of the in vivo-jetPEI solution were prepared in plastic tubes and frozen for use as control samples which were freshly thawed and diluted 15× with Milli-Q water (93.3 μL) prior to use.

The solutions of experimental samples and control samples were dispensed in a UV-compatible 96 well microplate (BRANDplates, pureGrade) as shown in Table 6 and were measured in triplicate.

TABLE 6
Solutions Used in Copper Assay.

| | Sample | Water | CuSO₄ |
|---|---|---|---|
| Sample | volume [μl] | volume [μl] | volume [μl] |
| In vivo-Jet LPEI (15x; | 8 | 92 | 100 |
| Control) | | | |
| LPEI-l-PEG-[Targeting | 8 | 92 | 100 |
| Fragment] | | | |

A blank consisting of 100 μL water and 100 μL CuSO₄ reagent was also measured in triplicate and the mean absorbance of the blank was subtracted from the absorbance values recorded for the experimental samples and the control sample. Solutions were left to react for 20 minutes at room temperature and their absorbance was then measured at 285 nm in a microplate reader (Spectramax Paradigm, Molecular Devices). Individual measurements were validated if the absorbance values were in the calibration range and were otherwise further diluted. Individual measurements were not validated if the coefficient of variation of the measurement was greater than 10.0% but were instead repeated. The measurement run was validated if the value of the control was within 10% of 150 mM. Concentrations were calculated using the following formula using the calibration slope k=0.0179:

$$c ( LPEI total ) [ mg / L ] = ( {A}_{corr , average} [ AU ] / k [ L * {mg}^{- 1} ] ) * ( 200 / 8 ) * dilution factor$$

Lyophilization. Lyophilization was performed on a freeze-drying device from Christ (Alpha 2-4 μLP Plus). Because of the presence of acetonitrile in some samples, the samples were cooled for about three minutes with liquid nitrogen at −196° C. before lyophilization.

Samples were lyophilized at −82° C. (condenser temperature) and 100 mbar (75 Torr). The time of lyophilization was adjusted based on the properties of the lyophilized compound.

Buffer Exchange general method. For preparation of triconjugates in a HEPES buffer, the resuspended TFA-lyophilisate solution was pH adjusted with NaOH to pH 6.5 before exchanging the buffer with 20 mM HEPES at pH 7.2.

For preparation of triconjugates in an acetate buffer, the resuspended TFA-lyophilisate solution was pH adjusted with NaOH to pH 4.5 before exchanging the buffer with 50 mM acetate at pH 4.3.

Detailed buffer exchange procedures that are compound specific are also provided below:

Tangential flow filtration (TFF) 2 kDa purification:

For the removal of TFA from DBCO-PEG₃₆-DUPA (Compound 37)·TFA salt, tangential flow filtration was performed on a Sartorius Slice Cassette composed of a peristaltic pump (Sartorius Stedim/Tandem Model 1082/SciLog, Inc.) with Masterflex® PharMed® tubing (Ref 06508-15) and Hydrosart membrane with a molecular weight cut-off (MWCO) of 2 kDa and a surface of 200 cm² (Sartorius Stedim/Sartocon Slice 200/Ref.: 3051441901E-SG/Lot: 90279123). The membrane was stored in 20-24% aq. EtOH.

The following TFF parameters were used: TMP: 2.0 bars; flow rate feed: 428 mL/min; flow rate permeate: 28 g/min.

For step-wise TFF, (1) 169 mL of DBCO-PEG₃₆-DUPA (Compound 37) solution were supplemented with 81 mL of 15 mM acetate pH 5.5. The solution was filtrated down to 50 mL by TFF. (2) The resulting 50 mL were supplemented with 250 mL of 15 mM acetate pH 5.5. The solution was filtrated down to 50 mL by TFF. (3) The resulting 50 mL were supplemented with 250 mL of 15 mM acetate pH 5.5. The solution was filtrated down to 50 mL by TFF. (4) The resulting 50 mL were supplemented with 250 mL of 15 mM acetate pH 5.5. The solution was filtrated down to 50 mL by TFF. (5) The resulting 50 mL were supplemented with 250 mL of 15 mM acetate pH 5.5. The solution was filtrated down to 50 mL by TFF. (6) The resulting 50 mL were supplemented with 250 mL of 15 mM acetate pH 5.5. The solution was filtrated down to 50 mL by TFF.

### Tangential Flow Filtration (TFF) 10 kDa Purification:

For the removal of TFA from LPEI-l-[N₃:DBCO]-PEG₃₆-DUPA (Compounds 31a and 31b)·TFA salt, tangential flow filtration experiments were performed on a Sartorius Slice Cassette composed of a peristaltic pump (Sartorius Stedim/Tandem Model 1082/SciLog, Inc.) with Masterflex® PharMed® tubing (Ref 06508-15) and Hydrosart membrane with a molecular weight cut-off (MWCO) of 10 kDa with a surface of 200 cm² (Sartorius Stedim/Sartocon Slice 200/Ref.: 3051443901E-SG/Lot: 01181123). The membrane was stored in 20-24% aq. EtOH.

The following TFF parameters were used: TMP: 1.6 bars; flow rate feed: 517 mL/min; flow rate permeate: 155 g/min.

For step-wise TFF, (1) 30 mL of LPEI-l-[N₃:DBCO]-PEG₃₆-DUPA (Compounds 31a and 31b)·TFA salt solution were supplemented with 220 mL of 20 mM HEPES pH 7.2. The solution was filtrated down to 50 mL by TFF. (2) The resulting 50 mL were supplemented with 250 mL of 20 mM HEPES pH 7.2. The solution was filtrated down to 50 mL by TFF. (3) The resulting 50 mL were supplemented with 250 mL of 20 mM HEPES pH 7.2. The solution was filtrated down to 50 mL by TFF. (4) The resulting 50 mL were supplemented with 250 mL of 20 mM HEPES pH 7.2. The solution was filtrated down to 50 mL by TFF. (5) The resulting 50 mL were supplemented with 250 mL of 20 mM HEPES pH 7.2. The solution was filtrated down to 50 mL by TFF.

### Polyplex Sizing Measurements and Characterization.

Triconjugates (e.g., LPEI-l-[N₃:DBCO]-PEG₃₆-DUPA) were complexed with nucleic acids to form polyplexes (e.g., LPEI-l-[N₃:DBCO]-PEG₃₆-DUPA:hIL-2 mRNA). The N/P ratio of the polyplexes, as referred herein, corresponds to the molar ratio of the nitrogen (N) content of the triconjugate to the phosphorus (P) content of nucleic acid measured prior to preparing polyplexes by mixing at the specified N/P ratio. Polyplex size distribution and ζ-potential were measured by DLS and ELS according to Hickey et al., J. Control. Release, 2015, 219, 536-47. The size of the polyplexes was measured by DLS with a Zetasizer Nano ZS instrument (Malvern Instruments Ltd., UK), working at 633 nm at 25° C. and equipped with a backscatter detector (173°), for example in HBG buffer (20 mM HEPES, 5% glucose, pH 7.2). Each sample was measured in triplicate. Briefly, polyplexes in HBG or HPS buffer were transferred into a quartz cuvette, typically and preferably using particle RI of 1.59 and absorption of 0.01 in HBG or 5% glucose (wt/vol) at 25° C. with viscosity of (0.98 mPa·s or 1.078 mPa·s) and RI of 1.330. Measurements were made using a 1730 Backscatter angle of detection previously equilibrated to 25° C. for at least 30 seconds, typically and preferably 60 seconds in triplicate, each with automatic run duration, without delay between measurements. Each measurement was performed seeking optimum position with an automatic attenuation selection. Data was analyzed using a General-Purpose model with normal resolution. The calculations for particle size and PDI are determined according to the ISO standard document ISO 22412:2017. The ζ-potential of polyplexes was measured by phase-analysis light scattering (PALS) (for example in HBG buffer at 25° C.), and/or electrophoretic light scattering (ELS) as described by instrument supplier (https://www.malvempanalytical.com/en/products/technology/light-scattering/electrophoretic-light-scattering). Briefly, polyplex samples in the indicated formulation buffer (e.g. 5% glucose) were transferred into a folded capillary cell and measured in 3-5 replicates. For nanoparticle material, settings of polystyrene latex were used: R.I. of 1.59 and absorption of 0.01. For dispersant, the experimentally determined viscosity of the formulation buffer were used (e.g. R.I. of 1.33 and viscosity 1.078 mPa·s for 5% glucose). Measurements were performed after at least 30 s incubation at 25° C. using the auto mode.

### Example 1

### Synthesis of LPEI-l-[N₃:DBCO]-PEG₂₄-hEGF (Compounds 1a and 1b)

LPEI-l-[N₃:DBCO]-PEG₂₄-hEGF was synthesized as a mixture of regioisomers 1a and 1b in two steps according to the schemes below. In the first step, human epidermal growth factor (hEGF) was coupled to dibenzoazacyclooctyne-24(ethylene glycol)-propionyl 2,3,5,6-tetrafluorophenol ester (DBCO-PEG₂₄-TFP; Compound 2) in 20 mM HEPES buffer to produce DBCO-PEG₂₄-hEGF (Compound 3). In the second step, DBCO-PEG₂₄-hEGF was conjugated to LPEI-N₃ to produce LPEI-l-[N₃:DBCO]-PEG₂₄-hEGF (Compounds (1a and 1b).

### Step 1: Synthesis of DBCO-PEG₂₄-hEGF (Compound 3)

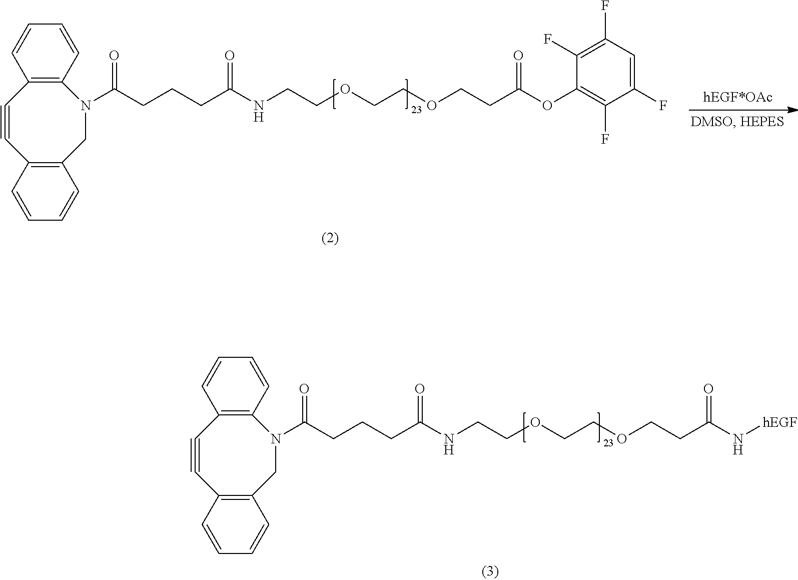

Human epidermal growth factor (hEGF acetate salt, 152.6 mg, 24.5 mmol; MW=6216.01 g/mol; CBL Patras, Greece) was weighed in a 250 mL round-bottom flask. 75 mL of 20 mM HEPES (pH 7.4) were added to the hEGF powder to obtain a 2 mg/mL solution of hEGF protein. The solution was agitated by magnetic stirring for 10 minutes until complete dissolution of the protein. The pH was adjusted to pH 7.5 with 150 mL of 1M NaOH and 60 mL of 5M NaOH. The purity of the solution was determined by UV spectrophotometry at 280 nm and the effective concentration of protein was found to be 0.23 mM (17.2 mmol).

Dibenzoazacyclooctyne-24(ethylene glycol)-propionyl 2,3,5,6-tetrafluorophenol ester (DBCO-PEG₂₄-TFP; Compound 2; 100.2 mg, 62.8 mmol, MW=1,595.75 g/mol; Iris Biotech, Germany) was weighed in a 15 mL Falcon tube and dissolved in 6.0 mL DMSO to form a 10 mM stock solution. The purity of the DBCO-PEG₂₄-TFP solution was measured by UV spectrophotometry at 309 nm after a 40-fold dilution with DMSO. The effective concentration of DBCO-PEG₂₄-TFP was found to be 9.32 mM (89%, 55.9 mmol).

DBCO-PEG₂₄-TFP (Compound 2; 3.68 mL, 34.3 mmol, 2.0 eq of the stock solution) was slowly added to the hEGF solution under magnetic stirring at room temperature. After 2.5 hours an additional 0.92 mL of the DBCO-PEG-TFP (Compound 2) stock solution (8.6 mmol, 0.5 eq) were added to the reaction mixture. The solution was left to react for a further 30 minutes. The reaction mixture was transferred into two 50 mL Falcon tubes and kept at 4° C. for 2 hours prior to purification.

The reaction mixture (79 mL) was purified in 4 runs using the Waters preparative chromatography system. Before each run, the solutions were supplemented with acetonitrile to reach 10% ACN in order to have the same composition as the eluant at the start of the preparative chromatography. Pooled fractions were collected for lyophilization. A total of about 273 mL of isolated DBCO-PEG₂₄-hEGF (Compound 3) were recovered in 50 mL Falcon tubes (3.4-fold dilution). The four pools were mixed and the combined samples were analyzed by C8-RP-HPLC and stored under argon at −80° C. prior to lyophilization.

The isolated DBCO-PEG₂₄-hEGF (Compound 3) was cooled in liquid nitrogen for about 3 min before lyophilization. A fluffy lyophilizate (70 mg, 46% yield in hEGF, 89% yield in DBCO, [(M+6H)⁶⁺]/6=1274.42, monoisotopic mass [Da] measured 7640.47, monoisotopic mass [Da] calculated 7640.47) was recovered and stored under argon at −80° C.

### Step 2: Synthesis of LPEI-l-[N₃:DBCO]-PEG₂₄-hEGF (Compounds 1a and 1b)

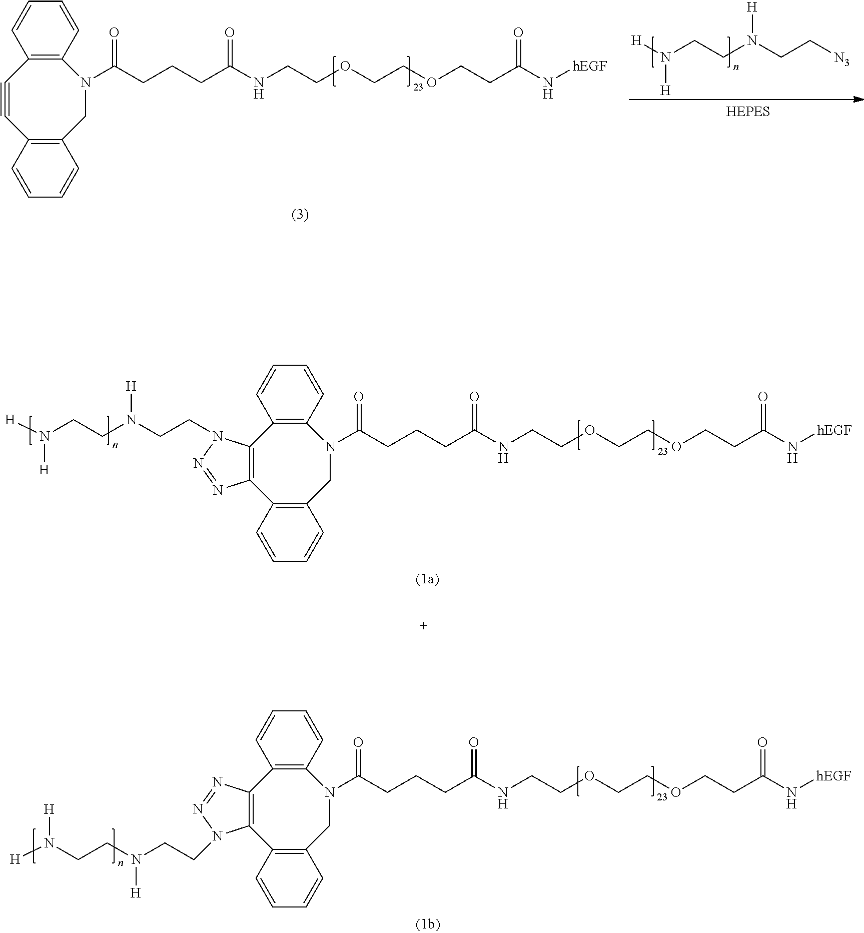

DBCO-PEG₂₄-hEGF lyophilisate (Compound 3; ˜43 mg) was weighed into a 15 mL Falcon tube and dissolved in 5.4 mL of 20 mM HEPES (pH 6.5; 8 mg/mL solution). The pH after dissolution was 3.9 and was adjusted to pH 4.5 with 3 μL of 5M NaOH. As the solution became cloudy, 15 μL of HCl 1M were used to re-dissolve the precipitate and the solution became clear again. The final pH of the solution was 3.7. The solution was filtered using 0.45 m nylon filters (13 mm nylon membrane from Exapure, Germany) to give ˜4.7 mL of DBCO-PEG₂₄-hEGF (Compound 3) solution. The effective concentration of DBCO-PEG₂₄-hEGF (Compound 3) was measured by UV spectrophotometry at 309 nm after a 20-fold dilution with H₂O. The assay gave a compound content of ˜86% with a concentration of 0.89 mM (4.2 mol).

LPEI-N₃ (199.5 mg) was weighed in a 50 mL Falcon tube and dissolved in 10 mL MilliQ water pH 2.2 (20 mg/mL solution). 350 μL of 1M HCl were added to help solubilize the LPEI-N₃. The solution was sonicated for about three minutes and heated to 70° C. until the LPEI-N₃ was completely dissolved. The measured pH was 7.8 and 800 μL of 1M HCl+300 μL of 1M NaOH were used to adjust the pH to 4.6. The concentration of LPEI-N₃ was measured by copper assay and a purity of ˜69% was found. The effective concentration of the solution was 0.55 mM.

In a 50 mL Falcon tube, DBCO-PEG₂₄-hEGF (Compound 3) solution (4.7 mL, 4.2 mol), LPEI-N₃ solution (7.6 mL, 4.2 μmol) and a NaCl solution (400 μL, 4.8 M) were mixed and left to react on a Stuart rotator at 20 rpm at room temperature. Samples were regularly taken for analytical HPLC monitoring of the reaction at 240 nm and 309 nm. After 95 hours no significant further conversion was evident and the reaction was stopped. Based on the decrease of the peak area, 55-60% of DBCO-PEG₂₄-hEGF (Compound 3) was consumed. About 12.5 mL of solution were recovered and the pH was measured to be 4.9. The solution was stored at −80° C. under argon prior to purification.

The reaction mixture (about 12.5 mL) was brought to room temperature and treated with 1.4 mL of acetonitrile and 15 μL TFA. The solution was filtered with 0.4 μM filters before purification using PuriFlash RP preparative chromatography. The fractions containing pure products were lyophilized, weighed, and analyzed by RP-HPLC, copper assay, and UV spectrophotometry at 280 nm. The retention time of the LPEI-l-[N₃:DBCO]-PEG₂₄-hEGF (Compounds 1a and 1b) in the analytical RP-HPLC analysis was 5.6-5.8 min. 29 mg of a mixture of LPEI-l-[N₃:DBCO]-PEG₂₄-hEGF (Compounds 1a and 1b) trifluoroacetate, each with a LPEI:hEGF ratio of 1:1 and no further impurities was isolated (12% overall yield in LPEI).

### Step 3: Exchanging TFA Salt for HEPES Buffer

To exchange TFA with HEPES, 11.5 mg of lyophilized LPEI-l-[N₃:DBCO]-PEG₂₄-hEGF (Compounds 1a and 1b) trifluoroacetate (w_{LPEI}=26%, −3 mg in total LPEI) were dissolved in 1.0 mL, 20 mM HEPES (pH 7.2) in a 2 mL Eppendorf tube. The initial pH was 3.5 and was adjusted to pH 7.2 with 8 μL of 5 M NaOH and 9 μL of 1 M HCl. An additional 483 μL of 20 mM HEPES (pH 7.2) was added to give a final volume of about 1.5 mL. The total concentration of LPEI was about 2 mg/mL. Three centrifugal filters were filled with 450 μL (1350 μL in total) of LPEI-l-[N₃:DBCO]-PEG₂₄-hEGF trifluoroacetate. The tubes were each centrifuged once at 14,000 g for 30 minutes. The supernatant was decanted, and the pellet re-suspended in 20 mM HEPES buffer (pH 7.2) at 25° C. The tubes were centrifuged again at 14,000 g for 30 minutes and the supernatant was decanted. The pellet was re-suspended in 20 mM HEPES buffer (pH 7.2) and re-centrifuged two additional times. About 1.3 mL of the solution of LPEI-l-[N₃:DBCO]-PEG₂₄-hEGF (Compounds 1a and 1b) as a HEPES salt were recovered at a concentration of 2.1 mg/mL of total LPEI.

### Step 4: Exchanging TFA Salt for Acetate Buffer

To exchange TFA with acetate, 12.5 mg of lyophilized LPEI-l-[N₃:DBCO]-PEG₂₄-hEGF (Compounds 1a and 1b) trifluoroacetate (w_{LPEI}=26%, −3 mg in total LPEI) were dissolved in 1.3 mL, 50 mM acetate buffer (pH 4.5) in a 2.0 mL Eppendorf tube. The initial pH was 4.0 and was adjusted to pH 4.5 with 3.5 μL of 5 M NaOH. The total concentration of LPEI was about 2 mg/mL. Four centrifugal filters were filled with 325 μL (1300 μL in total) of LPEI-l-[N₃:DBCO]-PEG₂₄-hEGF trifluoroacetate. The tubes were each centrifuged once at 14,000 g for 30 minutes. The supernatant was decanted, and the pellet re-suspended in 50 mM acetate buffer (pH 4.5) at 4° C. The tubes were centrifuged again at 14,000 g for 30 minutes and the supernatant was decanted. The pellet was re-suspended in 50 mM Acetate buffer (pH 4.5) and re-centrifuged two additional times. About 1.4 mL of the solution of LPEI-l-[N₃:DBCO]-PEG₂₄-hEGF (Compounds 1a and 1b) as an acetate salt were recovered at a concentration of 2.3 mg/mL of total LPEI.

### Example 2

### Synthesis of LPEI-l-[N₃:DBCO]-PEG₁₂-hEGF (Compounds 4a and 4b)

LPEI-l-[N₃:DBCO]-PEG₁₂-hEGF was synthesized as a mixture of regioisomers 4a and 4b in two steps according to the schemes below. In the first step, human epidermal growth factor (hEGF) was coupled to dibenzoazacyclooctyne-12(ethylene glycol)-propionyl 2,3,5,6-tetrafluorophenol ester (DBCO-PEG₁₂-TFP; Compound 5) in 20 mM HEPES buffer to produce DBCO-PEG₁₂-hEGF (Compound 6). In the second step, DBCO-PEG₁₂-hEGF (Compound 6) was conjugated to LPEI-N₃ to produce LPEI-l-[N₃:DBCO]-PEG₁₂-hEGF (Compounds 4a and 4b).

### Step 1: Synthesis of DBCO-PEG₁₂-hEGF (Compound 6)

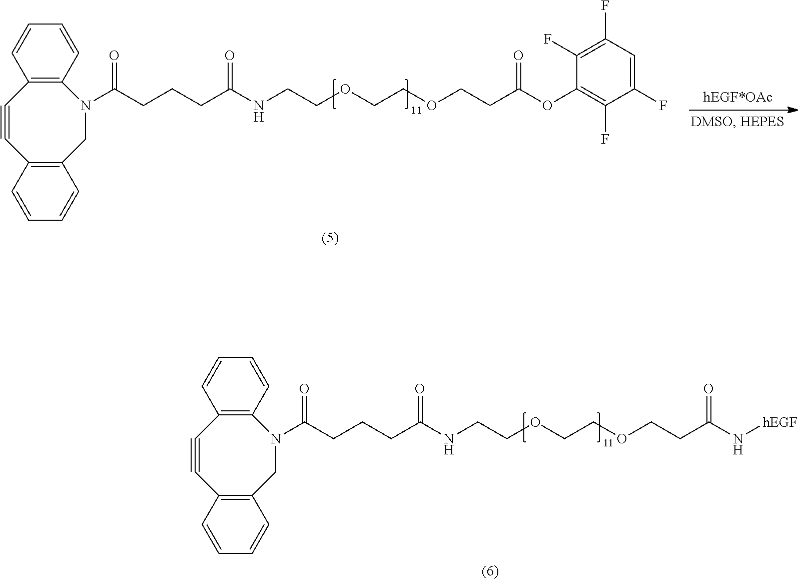

56 mg of dibenzoazacyclooctyne-12(ethylene glycol)-propionyl 2,3,5,6-tetrafluorophenol ester (DBCO-PEG₁₂-TFP; Compound 5; assay 96.3%; 51 mol pure product) were weighed in a 5 mL Eppendorf tube and dissolved in 2.6 mL DMSO (˜20 mM stock solution, pure product). The solution was manually mixed to dissolve DBCO-PEG₁₂-TFP.

148 mg (crude mass) of hEGF (Lot 5263, 87.1% peptide content; 21 mol) were weighed in a 100 mL round-bottom flask and dissolved in 75 mL 20 mM HEPES, pH 7.4. The solution was agitated by magnetic stirring for about 10 minutes to dissolve the protein and the pH of the solution was adjusted to 7.5 with 100 μL 5 M NaOH and 16 μL 6 M HCl.

2.08 mL of DBCO-PEG₁₂-TFP (Compound 5) stock solution (2 eq, 42 mol) were slowly added to the hEGF solution with stirring. After about 15 minutes, 8.6 mL of ACN were added to the reaction mixture (10% of final volume). After about 50 minutes, an additional 0.52 mL of DBCO-PEG₁₂-hEGF stock solution (0.5 eq Compound 5; 10 mol) was added to the reaction mixture and further stirred for 3 hours. The slightly cloudy reaction mixture was centrifuged at 15,000 g for five minutes prior to purification. 86 mL of reaction mixture (10% acetonitrile) were purified in one run using the PuriFlash RP-preparative Column system. Pooled fractions were collected and lyophilized (47 mg, 31% yield of Compound 6; [(M+5H)⁵⁺]/5=1186.37, (monoisotopic mass [Da] measured 7112.16, monoisotopic mass [Da] calculated 7112.18)).

### Step 2: LPEI-l-[N₃:DBCO]-PEG₁₂-hEGF (Compounds 4a and 4b)

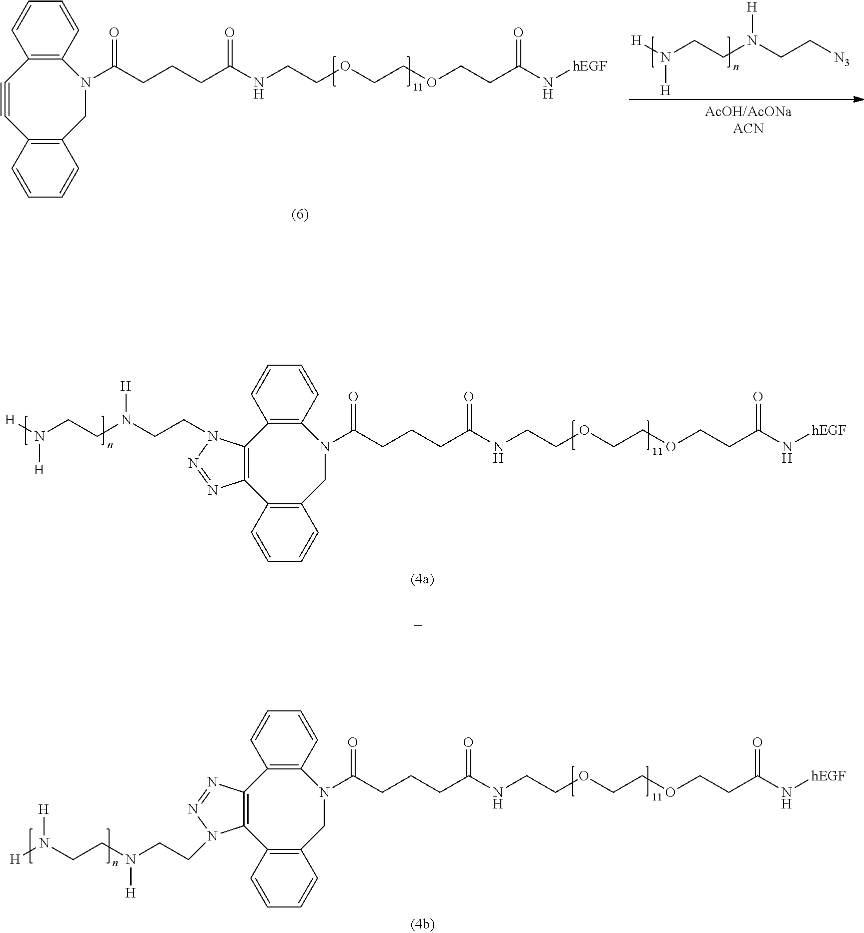

DBCO-PEG₁₂-hEGF lyophilisate (Compound 6; 46 mg, 6.5 mol) was dissolved in a mixture of 20 mL of 50 mM acetate, pH 4.0, and 2.2 mL acetonitrile (10% acetonitrile final volume). The pH of the solution was pH 4.2 and adjusted to 4.0 with 6 μL 6 M HCl. The final concentration of DBCO-PEG₁₂-hEGF (Compound 6) in solution was 2.3 mg/mL.

LPEI-N₃ (204 mg) were weighed in a 15 mL Falcon tube and dissolved in 10 mL 50 mM acetate, pH 4.0. The solution was heated to about 70° C. for about 2 minutes and 360 μL of 6 M HCl were added to help solubilize the LPEI-N₃ and to adjust the pH to 4.0 (19.7 mg/mL). The concentration of LPEI-N₃ (MW=22 kDa) was measured by copper assay and a purity of about 85% was determined. The effective concentration of the solution was 16.8 mg/mL (7.9 mol of LPEI-N₃ in solution).

The LPEI-N₃ solution (7.9 mol, 1.2 eq) was transferred to a 100 mL round-bottom flask equipped with a magnetic stirrer, and a DBCO-PEG₁₂-hEGF (Compound 6) solution (6.5 mol, 1.0 eq) was added. The reaction mixture was stirred at room temperature and protected from light for about 45 hours. Samples were regularly taken for monitoring and were diluted 10-fold with acetonitrile/H₂O (1:9) before injection. The reaction mixture (about 35 mL) was adjusted to contain about 6% (vol.) acetonitrile, and purified using the PuriFlash Pump injection system coupled to a preparative HPLC column. The pooled fractions containing pure products were lyophilized, weighed, and analyzed by RP-HPLC, copper assay, and UV spectrophotometry at 280 nm. 47 mg of a mixture of LPEI-l-[N₃:DBCO]-PEG₁₂-hEGF (Compounds 4a and 4b), each with a LPEI:hEGF ratio of 1:1 and no further impurities was isolated (7% overall yield in LPEI). Retention times of the LPEI-l-[N₃:DBCO]-PEG₁₂-hEGF in the analytical RP-HPLC analysis was 5.5-6.2 min.

### Example 3

### Synthesis of LPEI-l-[N₃:DBCO]-PEG₄-hEGF (Compounds 7a and 7b)

LPEI-l-[N₃:DBCO]-PEG₄-hEGF was synthesized as a mixture of regioisomers 7a and 7b in two steps according to the schemes below. In the first step, human epidermal growth factor (hEGF) was coupled to dibenzoazacyclooctyne-4(ethylene glycol)-propionyl 2,3,5,6-tetrafluorophenol ester (DBCO-PEG₄-TFP; Compound 8) in 20 mM HEPES buffer to produce DBCO-PEG₄-hEGF (Compound 9). In the second step, DBCO-PEG₄-hEGF (Compound 9) was conjugated to LPEI-N₃ to produce LPEI-l-[N₃:DBCO]-PEG₄-hEGF (Compounds 7a and 7b).

### Step 1: Synthesis of DBCO-PEG₄-hEGF (Compound 9)

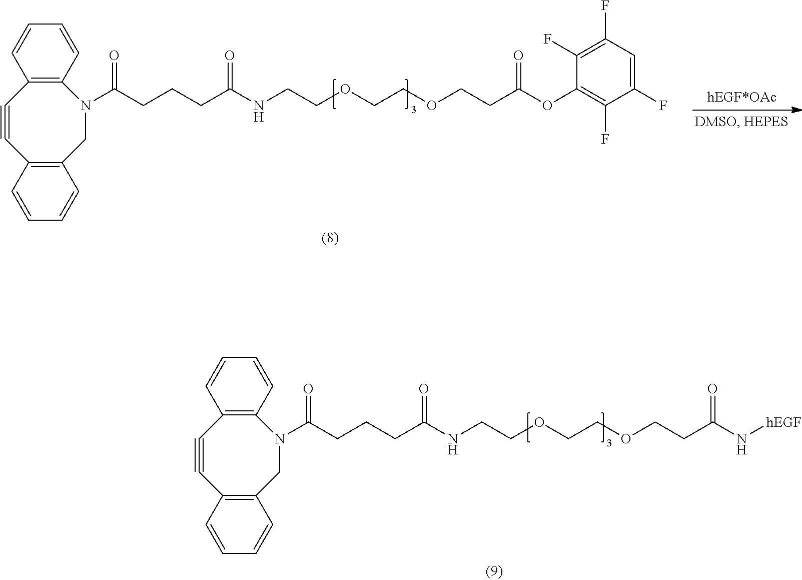

A solution of hEGF (150 mg, peptide content 87.1%, 24 μmol pure peptide, 1.0 eq, 0.29 mM) in 20 mM HEPES pH 7.5/ACN (9:1) (83.6 mL) was mixed with a solution of DBCO-PEG₄-TFP (Compound 8; 43 μmol, 1.8 eq, 20 mM) in DMSO (2.2 mL). The reaction mixture was incubated in a 100 mL round-bottom flask under magnetic stirring at room temperature and was monitored by RP-C₈-HPLC. After 25 minutes, the reaction mixture was supplemented with acetonitrile (10 mL) and after one and a half hours, the mixture was supplemented with additional DBCO-PEG₄-TFP (12 μmol, 0.5 eq, 20 mM). After a total of three hours, the reaction mixture was stored at 4° C. overnight. The reaction mixture was adjusted to 10% ACN and DBCO-PEG₄-hEGF was isolated following RP-C₁₈ preparative HPLC and lyophilization of pooled fractions. A solid (59 mg) was recovered and analyzed by HPLC-ESI⁺ qTOF mass spectrometry. The solid contained DBCO-PEG₄-hEGF (calculated monoisotopic mass: 6759.95 Da; measured: 6760.02 Da).

### Step 2: Synthesis of LPEI-l-[N₃:DBCO]-PEG₄-hEGF (Compounds 7a and 7b)

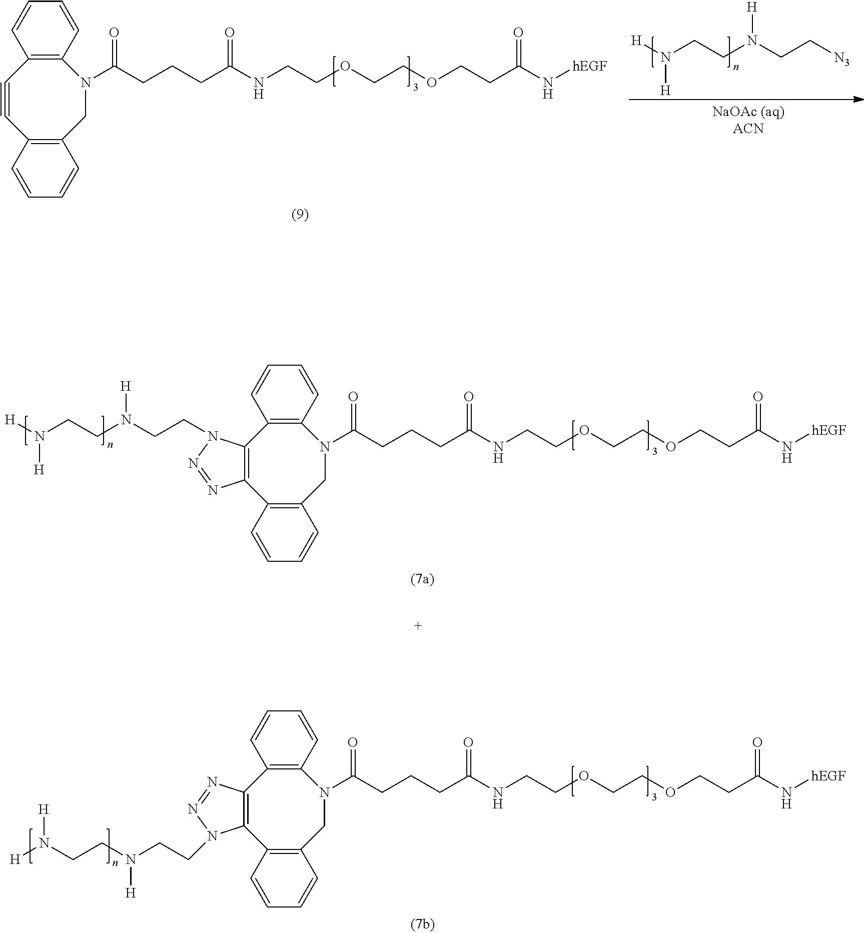

LPEI-N₃ solution (10 mL, 6.4 μmol, 1.0 eq, 0.64 mM) in 50 mM acetate buffer pH 4.0 was slowly added a solution of DBCO-PEG₄-hEGF (6.6 μmol, 1.0 eq, 0.30 mM) in 50 mM acetate pH 4.0/ACN (9:1) (22.3 mL). The reaction mixture was incubated in a 100 mL round-bottom flask under magnetic stirring at room temperature and protected from light and was monitored by RP-C₈-HPLC. After 45 hours of reaction, LPEI-l-[N₃:DBCO]-PEG₄-hEGF was isolated as a mixture of regioisomers 7a and 7b using RP-C₁₈ preparative HPLC. Pooled fractions were lyophilized (47 mg, fluffy white solid) and characterized by RP-C₈-HPLC, copper assay and spectrophotometry at 280 nm for determination of the hEGF content. Lyophilisate had a weight percentage in LPEI of 26% w/w and a LPEI to hEGF ratio of 1/1.0.

Step 3: Preparation of LPEI-l-[N₃:DBCO]-PEG₄-hEGF-HEPES salt LPEI-l-[N₃:DBCO]-PEG₄-hEGF (Compounds 7a and 7b) TFA salt (22.9 mg, w_{LPEI}=26%, 6.0 mg in total LPEI) were dissolved in 2.5 mL 20 mM HEPES pH 7.2. Six centrifugal filters (Amicon Ultra—0.5 mL, 10 kDa MWCO) were filled with 420 μL of LPEI-l-[N₃:DBCO]-PEG₄-hEGF solution each, centrifuged one time at 14′000 g for 30 minutes and then three times after addition of 400 μL 20 mM HEPES, pH 7.2. About 449 μL of LPEI-l-[N₃:DBCO]-PEG₄-hEGF-HEPES salt solution were recovered and supplemented with 1.2 mL 20 mM HEPES, pH 7.2. The concentration of the solution was determined by copper assay (3.6 mg/mL in total LPEI, ratio LPEI/hEGF=1/1.0).

### Step 4: Preparation of LPEI-l-[N₃:DBCO]-PEG₄-hEGF-acetate Salt

LPEI-l-[N₃:DBCO]-PEG₄-hEGF (Compounds 7a and 7b) TFA salt (13.2 mg, w_{LPEI}=26%, 3.6 mg in total LPEI) were dissolved in 1.7 mL 50 mM acetate pH 4.3. Four centrifugal filters (Amicon Ultra—0.5 mL, 10 kDa MWCO) were filled with 425 μL of LPEI-l-[N₃:DBCO]-PEG₄-hEGF solution each, centrifuged one time at 14′000 g for 30 minutes and then three times after addition of 400 μL 50 mM acetate pH 4.3. About 211 μL of LPEI-l-[N₃:DBCO]-PEG₄-hEGF-acetate salt solution were recovered and supplemented with 1.2 mL 50 mM acetate pH 4.3. The concentration of the solution was determined by copper assay (2.2 mg/mL in total LPEI, ratio LPEI/hEGF=1/1.0).

### Example 4

### Synthesis of LPEI-l-[N₃:DBCO]-PEG₂₄-DUPA (Compounds 10a and 10b)

LPEI-l-[N₃:DBCO]-PEG₂₄-DUPA was synthesized as a mixture of regioisomers 10a and 10b in two steps according to the schemes below. In the first step, DUPA-Aoc-Phe-Gly-Trp-Trp-Gly-Cys (Compound 12; SEQ ID NO:4) (prepared analogously as described in WO2015/173824 A1 and WO2019/063705 A1) was coupled to dibenzoazacyclooctyne-24(ethylene glycol)-maleimide (DBCO-PEG₂₄-MAL; Compound 11) by Michael addition to prepare DBCO-PEG₂₄-DUPA (Compound 13). In the second step, DBCO-PEG₂₄-DUPA (Compound 13) was conjugated to LPEI-N₃ to produce LPEI-l-[N₃:DBCO]-PEG₂₄-DUPA (Compounds 10a and 10b).

### Step 1: Synthesis of DBCO-PEG₂₄-DUPA (Compound 13)

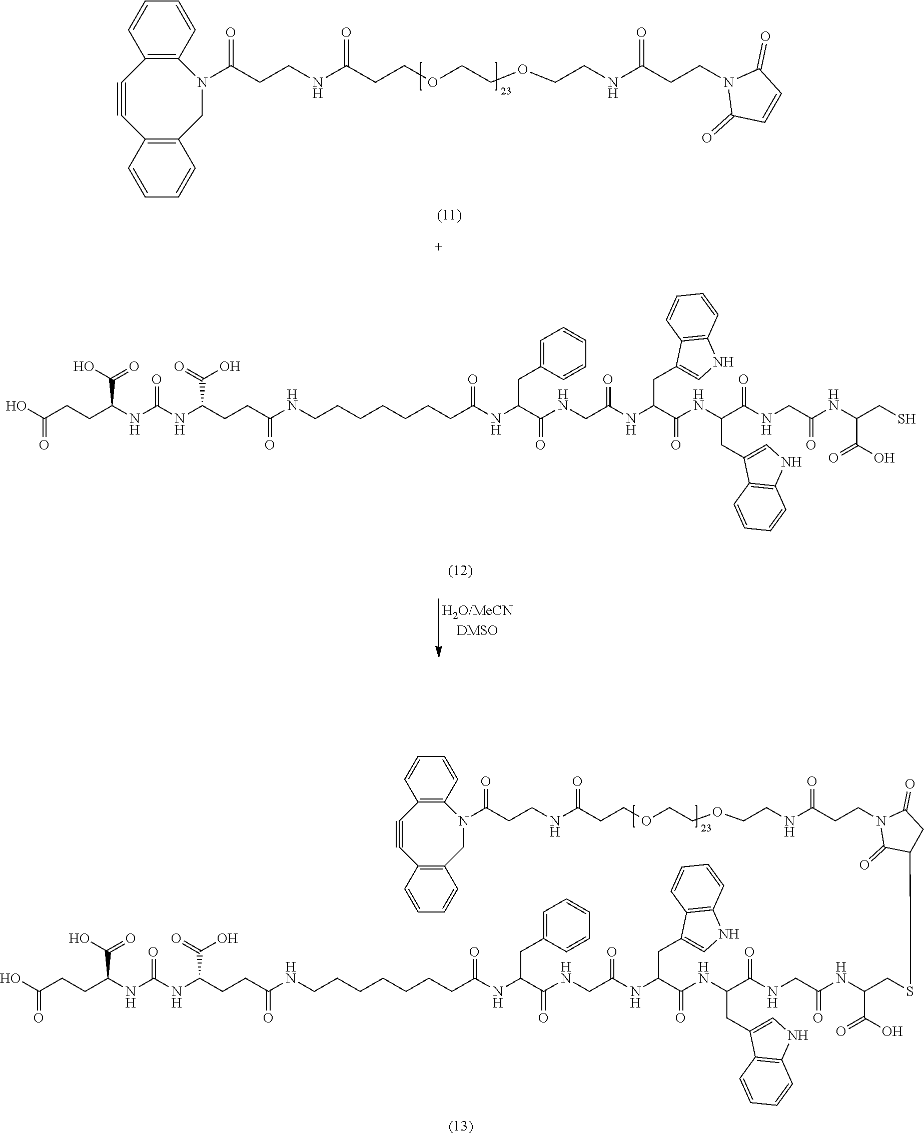

18.06 mg (crude mass) of DUPA-Aoc-Phe-Gly-Trp-Trp-Gly-Cys (Compound 12; 15 mol pure theoretical peptide content) were weighed in a 50 mL Falcon tube and dissolved in 9 mL H₂O/25% ACN (2.0 mg/mL stock solution). The solution was sonicated for about 15 seconds to help dissolve the DUPA-Aoc-Phe-Gly-Trp-Trp-Gly-Cys (Compound 12). The pH of the solution was adjusted to 3.5 with 8.5 μL 6 M HCl.

21.38 mg (crude mass) of DBCO-PEG₂₄-MAL (Compound 11; 13 mol pure product) were weighed in a 1.5 mL Eppendorf tube and dissolved in 650 μL DMSO (20 mM pure product). In the 50 mL Falcon tube containing the Compound 12 solution (15 mol, 1.5 eq), 500 μL of the DBCO-PEG₂₄-MAL (Compound 11) stock solution (10 mol, 1.0 eq) were added. The reaction mixture was protected from light and incubated on a Stuart rotator (20 rpm) for about 20 hours (RT). The reaction was monitored by C8-RP-HPLC and was continued up to complete conversion of DBCO-PEG₂₄-MAL (Compound 11). The identity of the DBCO-PEG₂₄-DUPA (Compound 13) produced by the reaction was confirmed by LC-MS (C8-RP-HPLC coupled with ESI-qTOF MS) analysis ((M+2H)2+]/2=1377.16, monoisotopic mass [Da] measured 2752.30, monoisotopic mass [Da] calculated 2752.30). The reaction was not quenched or purified and was used directly in Step 2.

### Step 2: Synthesis of LPEI-l-[N₃:DBCO]-PEG₂₄-DUPA (Compounds 10a and 10b)

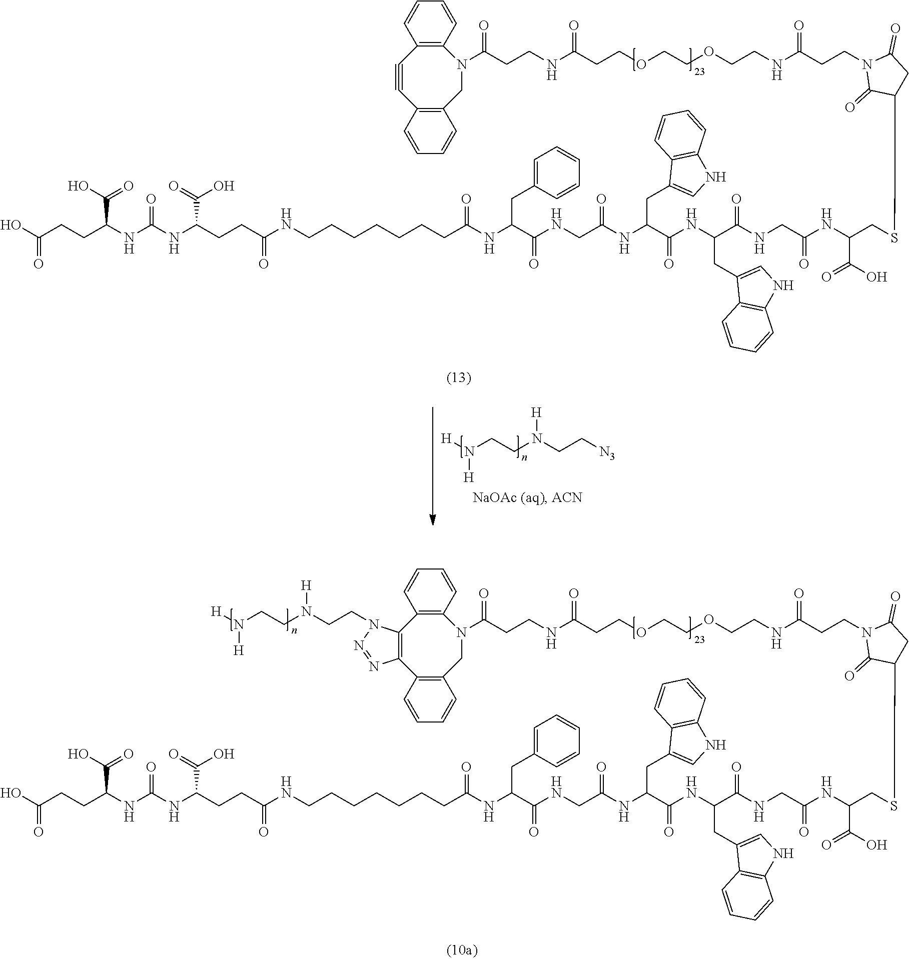

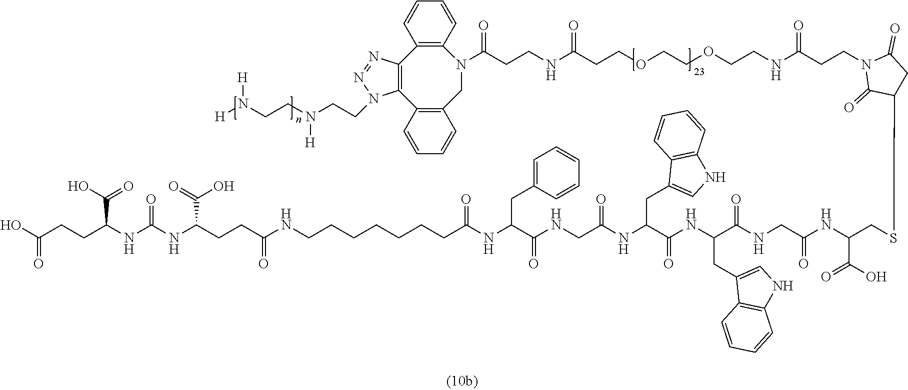

201.8 mg (crude mass) of LPEI-N₃ were weighed in a 15 mL Falcon tube and dissolved in 8 mL of 50 mM acetate buffer, pH 4.0. The pH of the solution was adjusted to 3.5 with 375 l of 6 M HCl, heated to 70° C., and sonicated for about three minutes to fully dissolve the LPEI particles. The solution was assayed using the copper assay and a concentration of 17.8 mg/mL total LPEI (0.811 mM) was measured (74% assay of LPEI-N₃).

8.3 mL of LPEI-N₃ solution (7 mol, 1.0 eq) were transferred to a 50 mL Falcon tube and mixed with 6.5 mL of the DBCO-PEG₂₄-DUPA (Compound 13) preparation of Step 1 (7 mol, 1.0 eq). As the reaction mixture became cloudy, 2 mL of acetonitrile were added (about 22% ACN final volume). The solution was degassed with argon for about 30 seconds.

The mixture of LPEI-N₃ and DBCO-PEG₂₄-DUPA was incubated for about 70 hours (RT) on a Stuart rotator (20 rpm), protected from light, and monitored by RP-C8-THPLC. After about three hours, white precipitates were visible in the solution and the reaction mixture gave a sweet, fruity odour.

Prior to preparative separation, the reaction mixture (˜16 mL) was diluted with 20 mL of H₂O containing 0.1% TFA to reduce the acetonitrile percentage to about 10%. The solution was centrifugated for 5 min at 15,000 g) and the supernatant was purified using the PuriFlash Preparative RP-HPLC system.

The pooled fractions containing pure Compounds 10a and 10b were lyophilized, weighed, and analyzed by RP-HPLC, copper assay, and UV spectrophotometry at 280 nm. 28 mg of LPEI-l-[N₃:DBCO]-PEG₂₄-DUPA (Compounds 10a and 10b), each with a LPEI:DUPA ratio of 1:1 and no further impurities was isolated (7% overall yield in LPEI). The retention time of the LPEI-l-[N₃:DBCO]-PEG₂₄. DUPA (Compounds 10a and 10b) in the analytical RP-HPLC analysis was 5.4-6.4 min with a maximum at 5.5 min.

### Example 5

### Synthesis of LPEI-l-[N₃:BCN]-PEG₁₂-hEGF (Compound 14)

LPEI-l-[N₃:BCN]-PEG₁₂-hEGF (Compound 14) is synthesized in two steps according to the schemes below. In the first step, human epidermal growth factor (hEGF) is coupled to endo-BCN-PEG₁₂-NHS ester (Compound 15) in 20 mM HEPES buffer to produce endo-BCN-PEG₁₂-hEGF (Compound 16). In the second step, endo-BCN-PEG₁₂-hEGF (Compound 16) is conjugated to LPEI-N₃ to produce LPEI-l-[N₃:BCN]-PEG₁₂-hEGF (Compound 14).

### Step 1: Synthesis of endo-BCN-PEG₁₂-hEGF

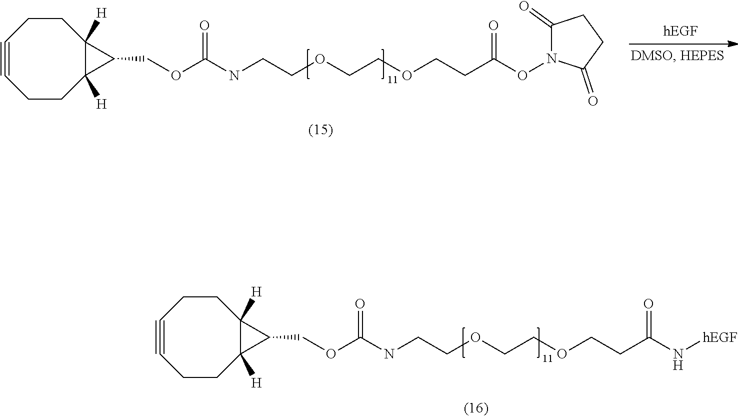

Endo-BCN-PEG₁₂-NHS (Compound 15; 21.8 mg, 23.9 mol, assay 97.7%) were weighed in a 5 mL Eppendorf tube and dissolved in 2.4 mL DMSO (10 mM stock solution, pure product). The solution was manually agitated to aid dissolution. hEGF (157 mg, 22.0 mol, 87.1% peptide content) was weighed in a 100 mL round-bottom flask and dissolved using 75 mL 20 mM HEPES, pH 7.4. The solution was agitated by magnetic stirring for about 10 minutes and adjusted to pH 7.4 with 60 μL 5 M NaOH. Endo-BCN-PEG₁₂-NHS (Compound 15) stock solution (2.2 mL, 22.0 mol, 1.0 eq) was slowly added to the magnetically stirred hEGF solution (22.0 mol, 1.0 eq). After −4 hours the reaction mixture was diluted to 10% ACN prior to PuriFlash purification. Pooled fractions from the preparative chromatography were analyzed by C8-RP-HPLC and lyophilized to give 43 mg endo-BCN-PEG₁₂-hEGF (Compound 16). The resulting Compound 16 lyophilizate was dissolved in 5.0 mL of 85% v/v 50 mM acetate (pH 4.0) containing 15% v/v ACN and further purified using 3 NAP-25 columns to remove hydrolyzed endo-BCN-PEG₁₂-OH impurity (identified by RP-C8-HPLC-MS (Single quadrupole, positive ionization)).

### Step 2: Synthesis of LPEI-l-[N₃:BCN]-PEG₁₂-hEGF

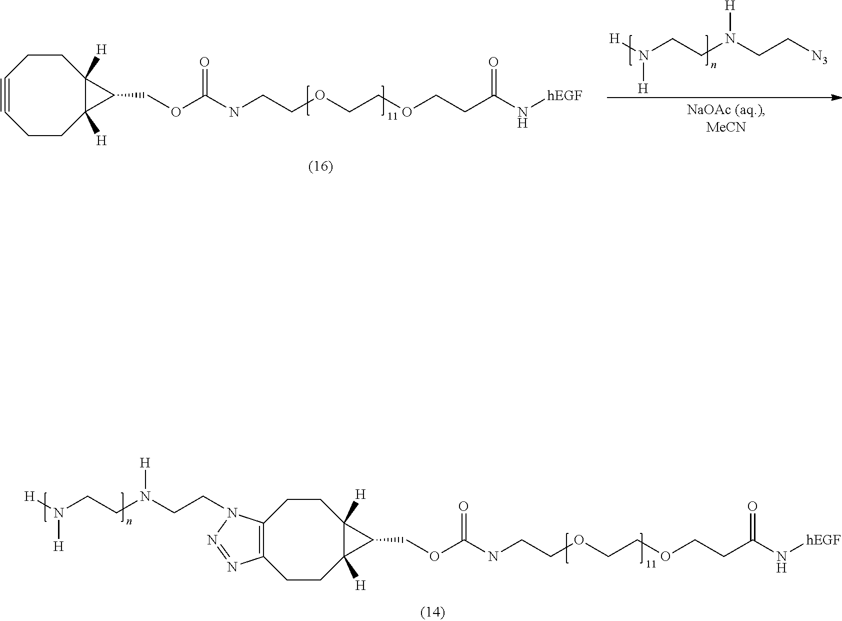

2.9 mL of LPEI-N₃ from a 0.77 mM stock solution (2.9 mL, 2.2 mol, 1.5 eq) was slowly added to a solution of endo-BCN-PEG₁₂-hEGF (Compound 16; 7 mL, 1.5 mol in peptide content, 1.0 eq) previously dissolved in 85% v/v 50 mM acetate, pH 4.0, 15% v/v ACN. The mixture was shaken for a total of 95 hours (40° C.) on a thermoshaker and protected from light. After ˜70 hours, an additional 0.85 mL (0.65 mol, 0.4 eq) of the LPEI-N₃ stock solution were added to the reaction mixture and the pH was adjusted to pH 4.0 using 5 M NaOH. Preparative chromatography was performed using an Agilent 1260 Infinity II preparative system to isolate the trifluoroacetate salt of Compound 14, which was subsequently lyophilized.

### Step 3: Preparation of LPEI-l-[N₃:BCN]-PEG₁₂-hEGF (Compound 14) acetate Salt

The lyophilized LPEI-l-[N₃:BCN]-PEG₁₂-hEGF-TFA salt produced above (˜50 mg) was mixed and solubilized with 4.5 mL 50 mM acetate (pH 4.5). The pH was adjusted to pH 4.3 using 5 M NaOH. Ten centrifugal filters (Amicon Ultra—0.5 mL, Merck Millipore Ltd.) were filled with 450 μL of LPEI-l-[N₃:BCN]-PEG₁₂-hEGF TFA salt solution each. They were centrifugated one time at 14′000 g for 30 minutes to remove buffer and then three times against 450 μL 50 mM acetate, pH 4.3 at 4° C. About 574 μL of the concentrated solution of LPEI-l-[N₃:BCN]-PEG₁₂-hEGF (Compound 14) acetate salt were recovered after buffer exchange and were supplemented with 3.0 mL 50 mM acetate, pH 4.3. A copper assay was performed on the final LPEI-l-[N₃:BCN]-PEG₁₂-hEGF (Compound 14) acetate salt solution (˜3.5 mL) and a 20 concentration of 2.1 mg/mL total LPEI was determined (ratio LPEI/hEGF=1/0.9).

### Example 6

### Synthesis of LPEI-l-[N₃:DBCO]-PEG₂₃-OCH₃ (Compounds 17a and 17b)

LPEI-l-[N₃:DBCO]-PEG₂₃-OCH₃ was synthesized in one step as a mixture of regioisomers 17a and 17b according to the scheme below. DBCO-PEG₂₃-OCH₃ (Compound 18) was coupled to LPEI-N₃ and purified over a 10 KDa filter using small scale, size exclusion centrifugation.

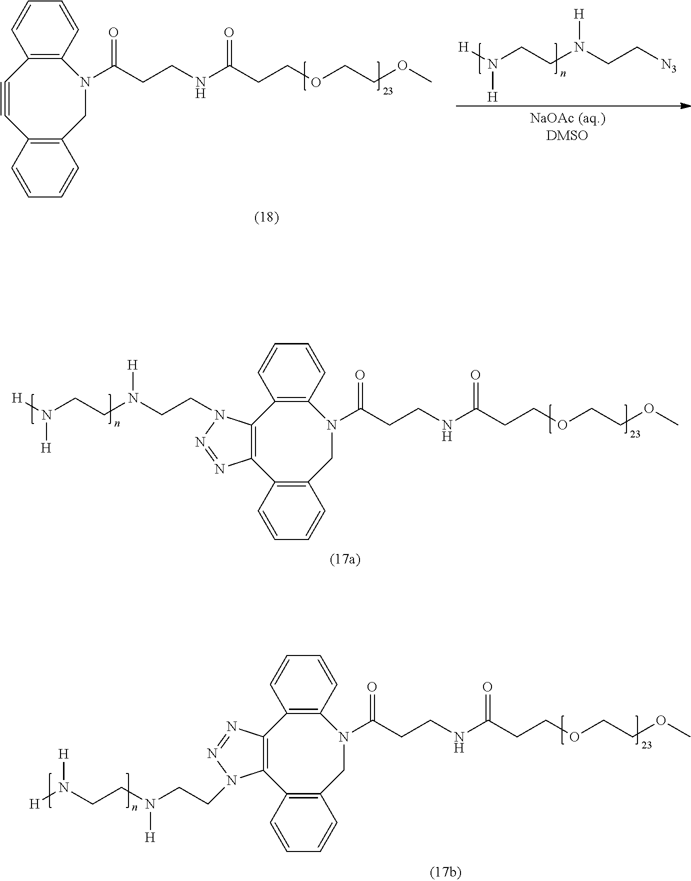

### Step 1: Synthesis of LPEI-l-[N₃:DBCO]-PEG₂₃-OCH₃ (Compounds 17a and 17b)

DBCO-PEG₂₃-OCH₃ (Compound 18, 3.25 mg, 2.4 mol, assay 98.9%) was weighed in a 1.5 mL Eppendorf tube and dissolved in 116 μL of DMSO (21 mM pure product). LPEI-N₃ (14.4 mg, MW=22 kDa) was weighed in a 1.5 mL Eppendorf tube and dissolved in 400 μL of 50 mM acetate buffer (pH 4.0). 6 M HCl (19 μL) was added to aid dissolution and to adjust to pH 3.5. Total LPEI concentration was measured by copper assay (25.1 mg/mL, 1.14 mM).

The LPEI-N₃ solution (400 μL, 0.46 mol, 1.0 eq) was transferred to a 1.5 mL Eppendorf tube and the DBCO-PEG₂₃-OCH₃ (Compound 18) solution (29 μL, 0.60 mol, 1.3 eq) was added to the reaction mixture and the resultant solution was kept at 40° C. for about 3 days.

The reaction mixture was purified over an Amicon centrifugal filter (10 kDa MWCO) against 50 mM acetate buffer (pH 4.0). Purified LPEI-l-[N₃:DBCO]-PEG₂₃-OCH₃ solution was further diluted with 2.8 mL of 50 mM acetate buffer (pH 4.0). The total LPEI content of the LPEI-l-[N₃:DBCO]-PEG₂₃-OCH₃ (Compound 17a and 17b) solution (˜3 mL) was measured by copper assay and found to be 1.3 mg/mL total LPEI. Based on the copper assay, the overall yield of reaction and purification was 39%.

### Comparative Example 1

### No Cycloaddition Reaction Between LPEI-OH and DBCO-PEG₂₃-OCH₃ (Compound 18)

To demonstrate the chemospecificity of the click-coupling reaction between an azide-modified LPEI fragment and a PEG fragment modified with an activated alkyne, a non-azide containing LPEI was treated with DBCO-PEG₂₃-OCH₃ (Compound 18) at pH 4 under the conditions set forth above in Example 6.

### Step 1: Treatment of DBCO-PEG₂₃-OCH3 with LPEI-OH

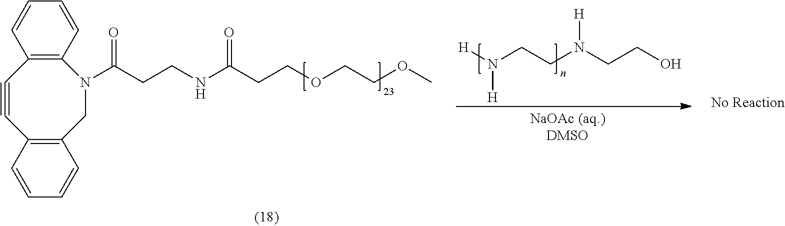

11.1 mg (crude mass) of non-azide-modified LPEI (α-methyl-ω-hydroxy-poly(iminoethylene), CH₃(NC₂H₅)ₙ—OH, 21 KDa, ChemCon GmbH, CAS No. 9002-98-6) were weighed in a 1.5 mL Eppendorf tube and dissolved in 400 μL of 50 mM acetate, pH 4.0. 26 μL of 6 M HCl were added to help dissolve and to adjust to pH 4. The concentration as measured by copper assay was 25.7 mg/mL (1.22 mM pure product). 400 μL of the LPEI solution (0.49 mol, 1.0 eq) were transferred in a 1.5 mL Eppendorf tube and 29 μL of DBCO-PEG₂₃-OCH₃ (Compound 18) solution (0.60 mol, 1.3 eq) were added to the reaction mixture. The solution was incubated at 40° C. for about 67 hours and monitored for product formation using analytical RP-HPLC. No product was evident at pH 4.

No reaction was observed using analytical RP-HPLC monitoring over 18 hours at room temperature. At higher pH, 5 evidence of a product was observed by analytical RP-HPLC, which was characterized as the hydroamination reaction product from coupling of the LPEI polyimine with the activated alkyne (F. Pohlki & S. Doye The catalytic hydroamination of alkynes Chem. Soc. Rev. 32. 104-114(2003)).

### Example 7

### Synthesis of LPEI-l-[N₃:MAL]-PEG_{2K}-DUPA (Compound 19)

LPEI-l-[N₃:MAL]-PEG_{2K}-DUPA (Compound 19), wherein the PEG fragment is a polydisperse fragment with a molecular weight of about 2,000, is synthesized in two steps according to the scheme below. In the first step, DUPA-Aoc-Phe-Gly-Trp-Trp-Gly-Cys (Compound 12; SEQ ID NO:4) (see Example 4), is coupled with half equivalent of MAL-PEG_{2K}-MAL (Compound 20) to prepare MAL-PEG_{2K}-DUPA (Compound 21). In the second step, MAL-PEG_{2K}-DUPA (Compound 21) is subjected to a 1,3-dipolar cycloaddition reaction with LPEI-N₃ according to the procedure taught by Zhu et al., Macromol. Res. 24, 793-799 (2016) to produce LPEI-l-[N₃:MAL]-PEG_{2K}-DUPA (Compound 19).

### Step 1: Synthesis of MAL-PEG_{2K}-DUPA (Compound 21)

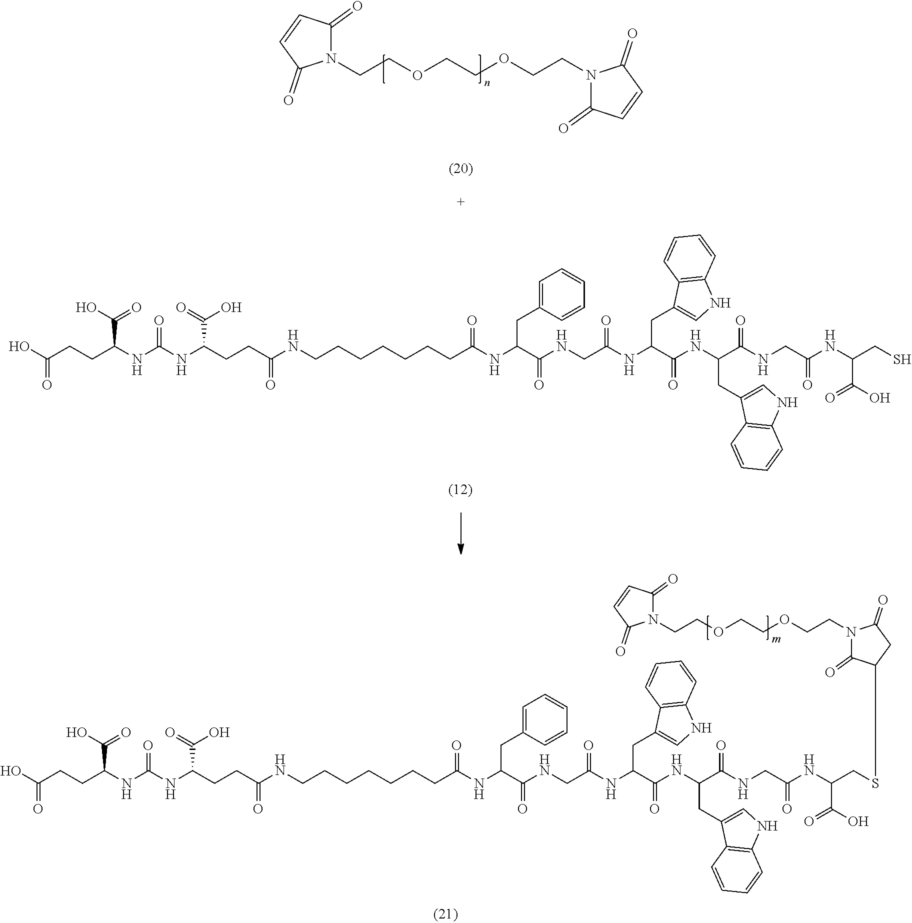

DUPA-Aoc-Phe-Gly-Trp-Trp-Gly-Cys (Compound 12; SEQ ID NO:4) is coupled with 0.5 equivalents of MAL-PEG_{2K}-MAL (Compound 20) to prepare MAL-PEG_{2K}-DUPA (Compound 21) according to the procedure of Example 4.

### Sten 2: Synthesis of LPEI-l-[N₃: MAL]-PEG_{2K}-DUPA (Compound 191

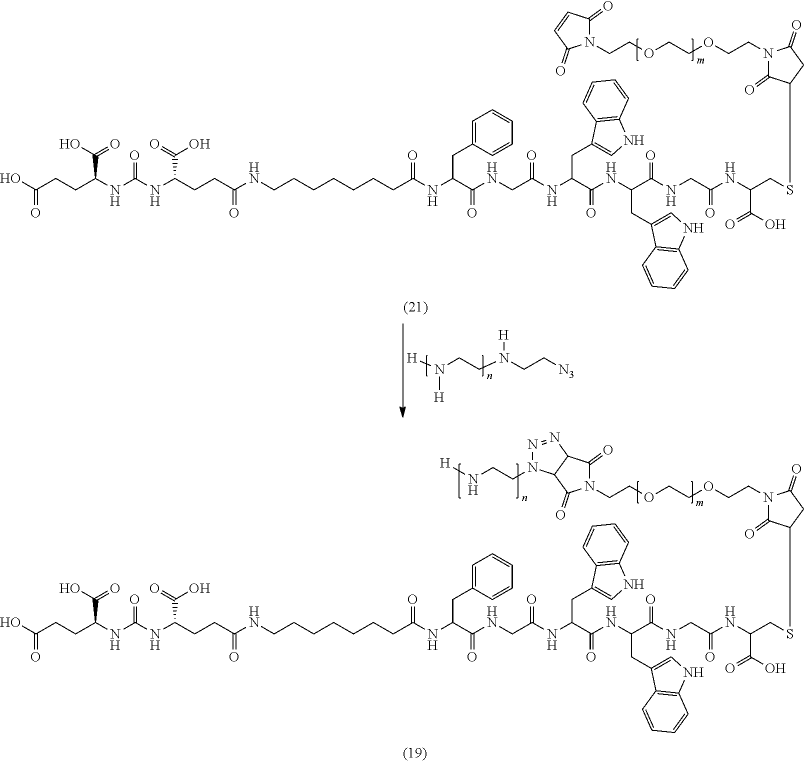

MAL-PEG_{2K}-DUPA (Compound 21) is subjected to a 1,3-dipolar cycloaddition reaction with LPEI-N₃ according to the procedure taught by Zhu et al., Macromol. Res. 24, 793-799 (2016) to produce LPEI-l-[N₃:MAL]-PEG_{2K}-DUPA (Compound 19).

### Example 8

### Synthesis of LPEI-l-[N₃:DBCO]-PEG₂₄-Folate (Compounds 22a and 22b)

LPEI-l-[N₃:DBCO]-PEG₂₄-Folate was synthesized as a mixture of regioisomers 22a and 22b in a multi-step procedure according to the schemes below. In the first step, folic acid (Compound 24) was functionalized at the gamma-Glu residue with a cysteamine spacer using a solid phase synthesis approach, analogous to that described by Atkinson et al., (J. Biol. Chem. 276(30) 27930-35 (2001)). The resultant folate-thiol (Compound 26) was coupled to dibenzoazacyclooctyne-24(ethylene glycol)-maleimide (DBCO-PEG₂₄-MAL; Compound 11) by Michael addition. In a next step, DBCO-PEG₂₄-Folate (Compound 27) was added to LPEI-N₃ in a [2+3]cycloaddition reaction to produce LPEI-l-[N₃:DBCO]-PEG₂₄-Folate (Compounds 22a and 22b).

### Step 1: Folic Acid Loading to Solid Phase Resin

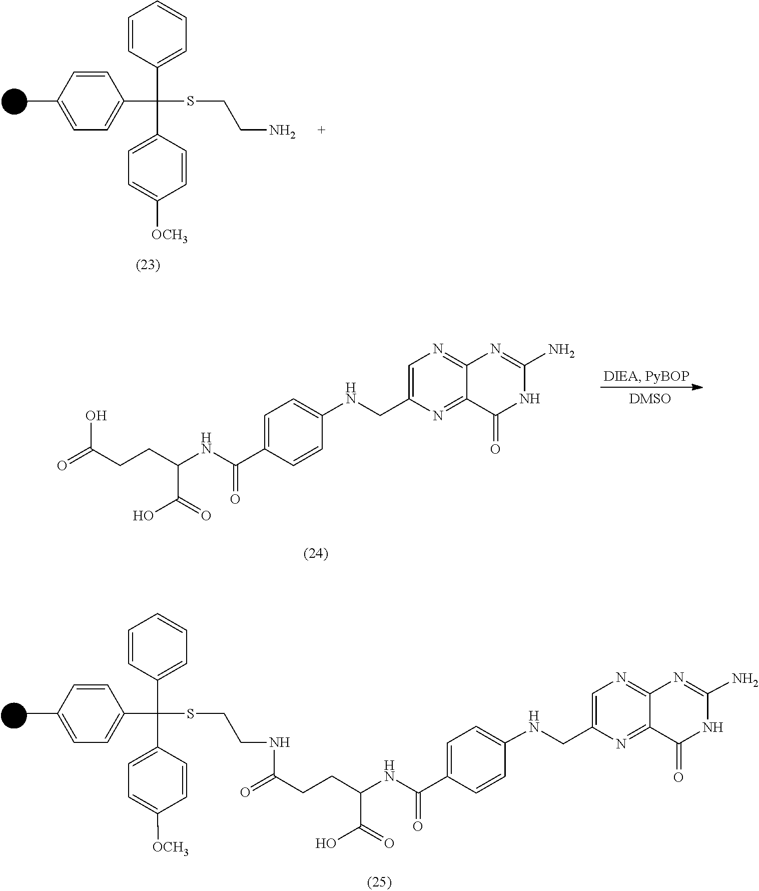 [SMILES: COc1ccc(C(SCCN)(c2ccccc2)c2ccc(C)cc2)cc1 ; Nc1nc2ncc(CNc3ccc(C(=O)NC(CCC(=O)O)C(=O)O)cc3)nc2c(=O)[nH]1 ; COc1ccc(C(SCCNC(=O)CCC(NC(=O)c2ccc(NCc3cnc4nc(N)[nH]c(=O)c4n3)cc2)C(=O)O)(c2ccccc2)c2ccc(C)cc2)cc1]

20 mL of DMSO was heated at 50° C. in a 50 mL Erlenmeyer and folic acid (Compound 24; 881.4 mg, 2.0 mmol, 5.0 eq) was slowly added under magnetic stirring. Dry cysteamine 4-methoxytrityl resin (Compound 23; 397.3 mg, 0.4 mmol, 1.0 equiv., 1.01 mmol/g) was added to a 50 mL Erlenmeyer flask and the previously prepared folic acid solution was added to the resin followed by the addition of DIEA (1018 μL, 6.0 mmol, 15.0 equiv) and PyBOP (1084.0 mg, 2.0 mmol, 5.0 equiv). The reaction mixture was stirred four hours at room temperature then transferred to a glass column and filtered over a glass frit and washed with DMSO (7×10 mL), DMF (5×10 mL), DCM (5×10 mL) and MeOH (5×10 mL). A TNBSA (picrylsulfonic acid) colour test on the sampled resin confirmed the absence of free amine.

### Step 2: Cleavage of the Folate-thiol from the Resin

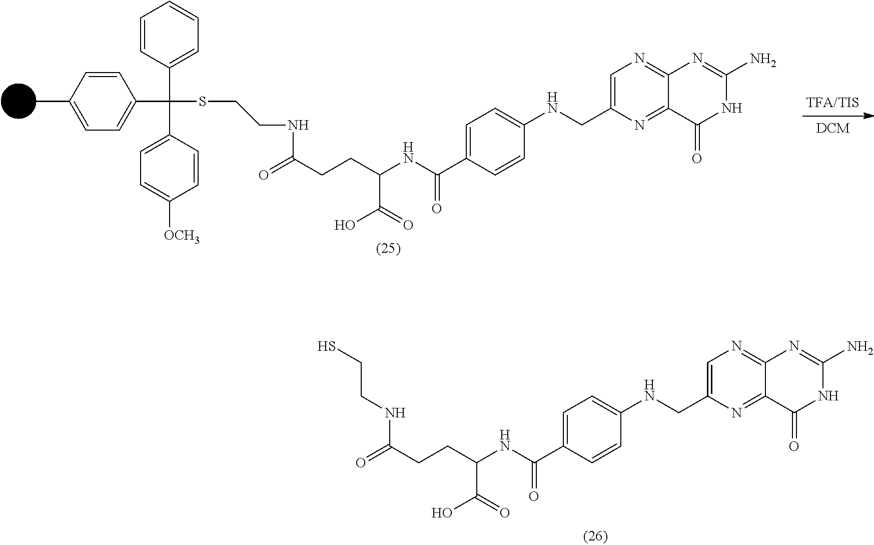 [SMILES: COc1ccc(C(SCCNC(=O)CCC(NC(=O)c2ccc(NCc3cnc4nc(N)[nH]c(=O)c4n3)cc2)C(=O)O)(c2ccccc2)c2ccc(C)cc2)cc1 ; Nc1nc2ncc(CNc3ccc(C(=O)NC(CCC(=O)NCCS)C(=O)O)cc3)nc2c(=O)[nH]1]

10 mL of DCM/TFA/TIS (92/3/5 v/v/v) was added to the folate-modified resin (Compound 25) of Step 1 in the glass column and the mixture was kept for 30 min with occasional swirling of the flask. The resin was filtered and washed (10 mL DCM/TFA (95/5 v/v) and the filtrate and washings were recovered and concentrated under reduced pressure. After concentration, the mixture was separated in two phases and the light phase was discarded. Crude product was precipitated by addition of 30 mL cold diethyl ether and washed twice with diethyl ether. The folate-SH (Compound 26) crude product was dried overnight under reduced pressure and confirmed by mass spectrometry. The thiol content of the crude Compound 26 was measured by Ellman's test yielding a positive result for free thiol. Mass spectrometry (ESI): C₂₁H₂₄N₈O₅S [M−H]⁻ 499.54, found 499.2.

### Step 3: Synthesis of DBCO-PEG₂₄-Folate (Compound 27)

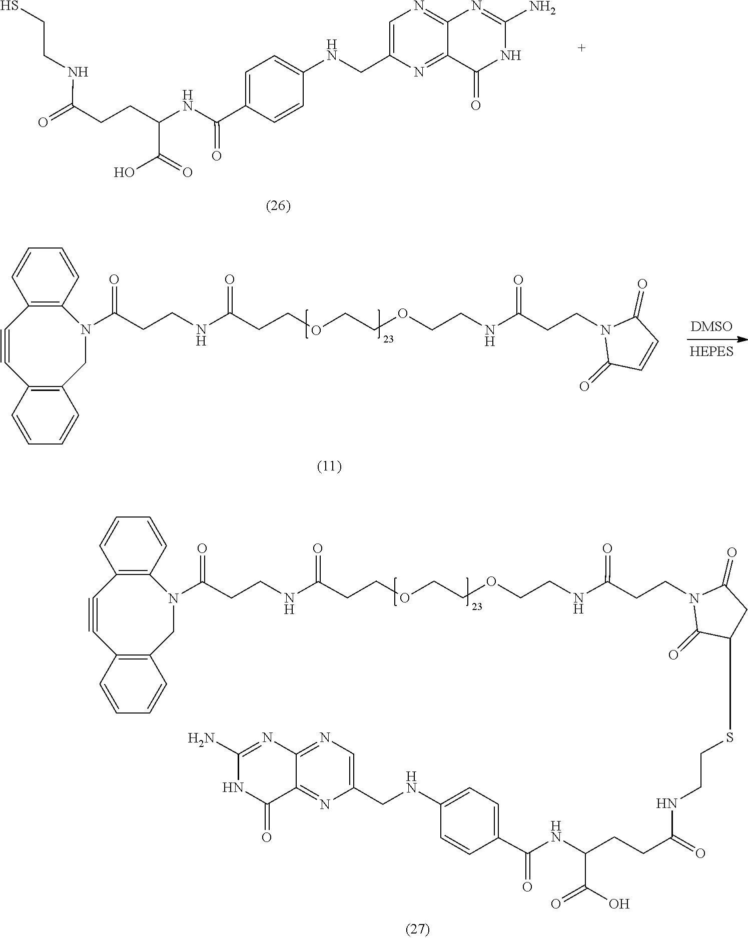

The folate-thiol (Compound 26) of Step 2 (16.0 mg, 29.4 mol, 1.7 eq) was dissolved in 8 mL DMSO in a round-bottom flask (2.0 mg/mL stock solution). The solution was sonicated to completely dissolve Compound 26 and diluted with 72 mL of 20 mM HEPES (pH 7.4). DBCO-PEG₂₄-MAL (Compound 11; see Example 4) (29.1 mg, 17.5 mol, assay 93.6%, 1.0 eq) was weighed in a 1.5 mL Eppendorf tube and dissolved in 875 μL DMSO (20 mM pure product stock solution). To the 80 mL round-bottom flask containing folate-thiol (Compound 26) solution (29.4 mol, 1.7 eq), the DBCO-PEG₂₄-MAL (Compound 11) stock solution (13 mol, 1.0 eq) was added slowly under magnetic stirring. The reaction mixture was kept at room temperature and protected from light for about one hour. DBCO-PEG₂₄-Folate (Compound 27) was purified by preparative chromatography using a Puriflash system and was confirmed by mass spectrometry. Mass spectrometry (ESI): [M+3H]³⁺ 2056.32, found 686.2.

### Step 4: Synthesis of LPEI-l-[N₃:DBCO]-PEG₂₄-Folate (Compounds 22a and 22b)

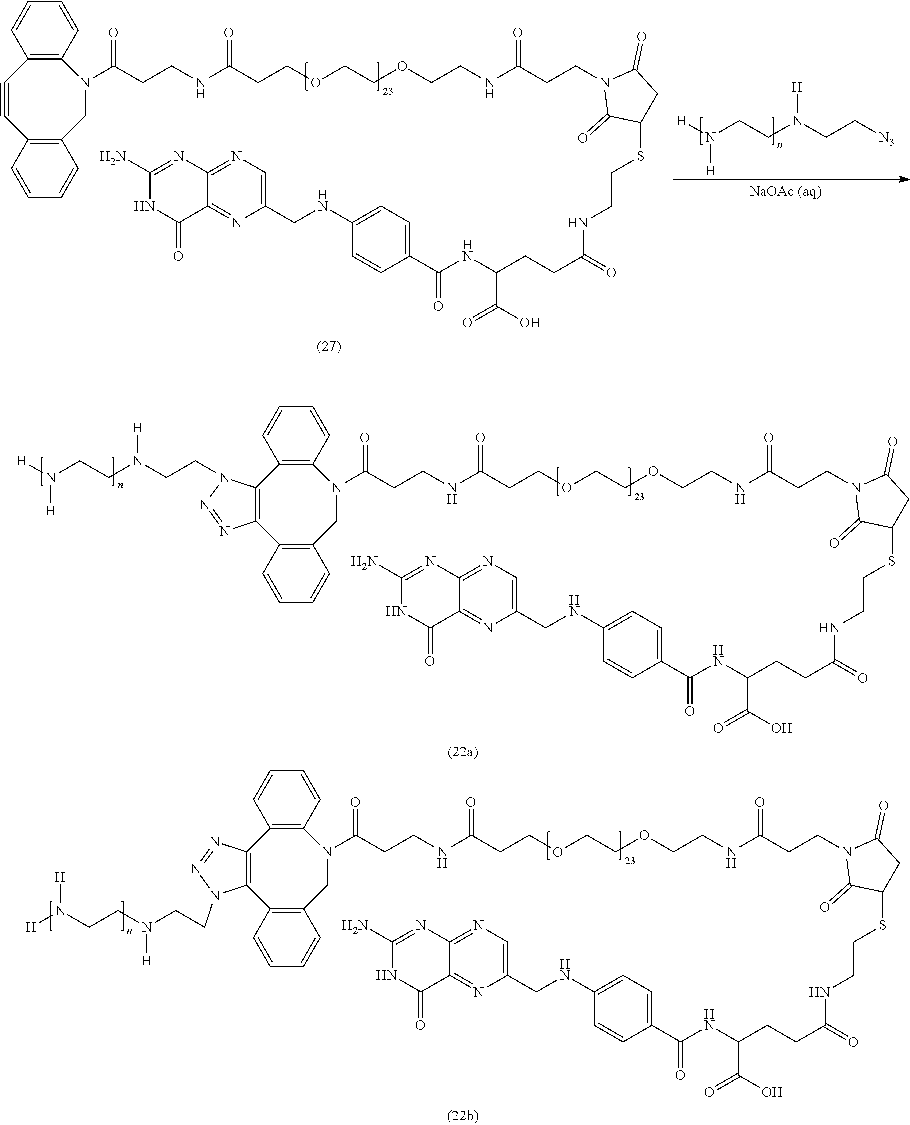

LPEI-N₃ stock (203.9 mg) was weighed in a 15 mL Falcon tube and dissolved in 8 mL of 50 mM acetate buffer (pH 4.0). The solution was acidified, heated to 70° C., sonicated to fully dissolve LPEI particles and adjusted to pH 4.0 with a total of 340 μL of 6 M HCl. The copper assay was performed on the solution to determine the total LPEI content of the LPEI-N₃ solution. LPEI-N₃ solution (8.3 mL, 6.7 mol, 1.0 eq) was transferred to a 50 mL Falcon tube and mixed with 1.5 mL of DBCO-PEG₂₄-Folate solution (Compound 27; 7 mol, 1.0 eq). The reaction mixture was degassed with argon and incubated for about 20 hours on a thermoshaker (40° C.) and protected from light.

Crude LPEI-l-[N₃:DBCO]-PEG₂₄-Folate was purified by preparative chromatography using a Puriflash system and isolated as a mixture of regioisomers 22a and 22b. Pooled fractions were measured for total LPEI content using the copper assay and for folate content by spectrophotometry (360 nm, ε=6′765 M⁻¹ cm⁻¹). Yield: 19 mg in LPEI content (copper assay); LPEI/folate ratio 1:1.

### Example 9

### Synthesis LPEI-l-[N₃:DBCO]-PEG₂₄-HER2-Affibody (Compounds 28a and 28b)

LPEI-l-[N₃:DBCO]-PEG₂₄-HER2-affibody was synthesized as a mixture of regioisomers 28a and 28b using a procedure analogous to the above method description for LPEI-l-[N₃:DBCO]-PEG₂₄-DUPA of Example 4, using a commercial cysteine-terminally modified affibody (Compound 29) in a Michael addition reaction to DBCO-PEG₂₄-MAL (Compound 11). The resulting DBCO-PEG₂₄-HER2-affibody (Compound 30) was coupled to LPEI-N3 in a [2+3]cycloaddition reaction to produce LPEI-l-[N₃:DBCO]-PEG₂₄-HER2-affibody (Compounds 28a and 28b).

### Step 1: Synthesis of DBCO-PEG₂₄-HER2

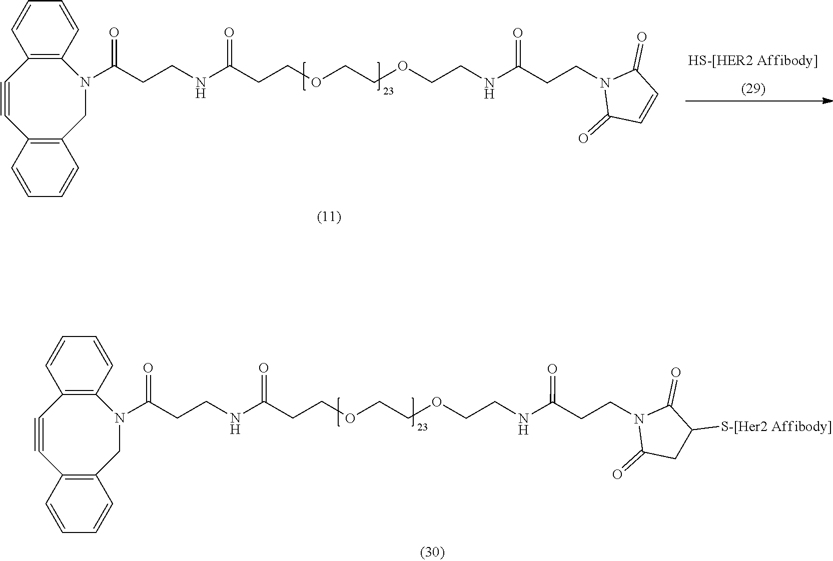

HER2 affibody (Compound 29; 4 mg, 0.29 mol, Mw=14 kDa) were weighed in a 5 mL Eppendorf tube. To reduce potential disulfide bonds within the HER2 affibody, a 0.5 M stock solution of DTT was prepared and was added to the HER2 affibody to a 20 mM final concentration of HER2 affibody. The reaction mixture was incubated for about 5 hours at room temperature. After reduction, DTT was removed with Sephadex G-25 columns with 20 mM HEPES (pH 7.4) as elution buffer. About 3.6 mg of purified HER2 affibody were recovered after NAP purification. Yield after NAP purification was estimated to be 90%.

A DBCO-PEG₂₄-MAL (Compound 11) stock solution was prepared by weighing 4.4 mg (crude mass) of Compound 11 in a 1.5 mL Eppendorf tube and adding 132 μL of DMSO to prepare a 20 mM stock solution. DBCO-PEG₂₄-MAL (Compound 11; 15 μL, 0.31 mol, 1.2 eq) stock solution was slowly added to the purified HER2-affibody solution (0.26 mol, 1.0 eq). The reaction mixture was incubated at room temperature on a Stuart rotator for about two hours and the reaction was monitored by RP-C8-HPLC at 280 nm and 309 nm.

The reaction mixture was purified with Amicon filters (10 kDa MWCO) to remove excess of DBCO-PEG₂₄-MAL (Compound 11) from the DBCO-PEG₂₄-HER2-affibody conjugate (Compound 30). Fourteen centrifugal filters (Amicon Ultra—0.5 mL, Merck Millipore Ltd.) were each filled with 429 μL of the reaction mixture. They were centrifugated one time at 14′000 g for 30 minutes to exchange buffer and remove residual DBCO-PEG₂₄-MAL (Compound 11) and then three times against 50 mM acetate buffer, pH 4.0 at 20° C. A concentrated solution of DBCO-PEG₂₄-HER2-affibody (Compound 30; 243 μL) was recovered after buffer exchange and supplemented with 1.0 mL 50 mM Acetate, pH 4.0. A total of ˜1.24 mL of purified DBCO-PEG₂₄-HER2-affibody (Compound 30) solution was obtained after the NAP purification step. The purified solution was analyzed by RP-C₈-HPLC and spectrophotometry at 309 nm with Nanodrop One C and a concentration of 118 M of DBCO was measured (˜0.15 mol).

### Step 2: Synthesis of LPEI-l-[N₃:DBCO]-PEG₂₄-HER2

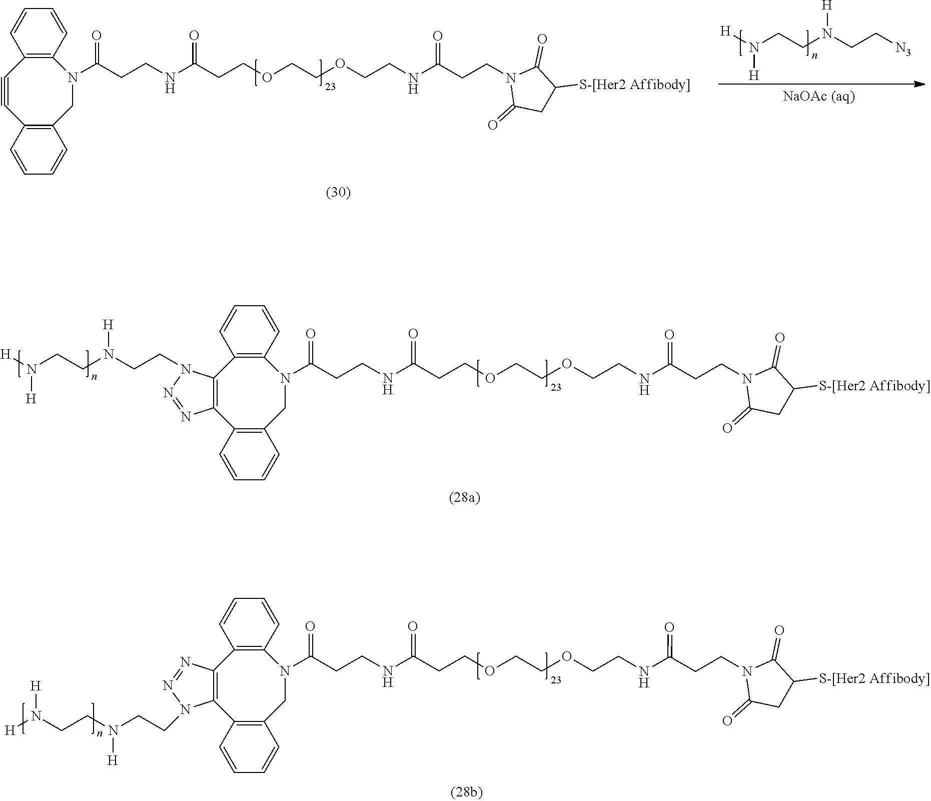

LPEI-N₃ (7.4 mg 0.34 mol, based on LPEI 72% (Cu assay) were weighed in a 15 mL Falcon tube and dissolved in 0.4 mL of 50 mM acetate buffer (pH 4.0). The solution was acidified, heated to 70° C., sonicated to fully dissolve LPEI particles, adjusted to pH 4.0 with a total of 15 μL of 6 M HCl, and degassed with argon. LPEI-N₃ from the stock solution (333 μL, 0.28 mol, 2.0 eq) was slowly added to the DBCO-PEG₂₄-HER2 (Compound 29) solution (0.14 mol, 1 eq). The reaction mixture was incubated for about 72 hours on a Stuart rotator. Additional LPEI-N₃ from a stock solution (215 μL, 0.14 μmol, 1.0 eq) was added to the reaction mixture and the solution was incubated for about 24 hours at 35° C. on a thermoshaker and monitored by RP-C₈-HPLC at 240 nm, 280 nm, and 309 nm with an ELSD detector. Prior to preparative chromatography, the percentage of acetonitrile of the reaction mixture was adjusted to 10% (final volume) with 189 μL of ACN and to 1% TFA (final volume) with 19 μL of TFA. The solution (˜1.7 mL) was supplemented with 1.0 mL of 90% v/v H₂O (0.1% TFA)/10% v/v ACN (0.1% TFA) and the total volume of sample was injected into the Agilent Prep-HPLC system. Pooled fractions were lyophilized to yield 4 mg LPEI-l-[N₃:DBCO]-PEG₂₄-HER2-affibody as a mixture of regioisomers 28a and 28b (overall yield in LPEI=6.9%; overall yield in anti-HER2 affibody=14%; 16% w/w in LPEI; ratio LPEI:DUPA=1/1.4).

### Step 3: Preparation of HEPES Salt Form

The lyophilized LPEI-l-[N₃:DBCO]-PEG₂₄-HER2-affibody (Compounds 28a and 28b) was dissolved in 0.8 mL 20 mM HEPES pH 7.2 in a 1.5 mL Eppendorf tube. The pH was adjusted to pH 7.2 with 5 M NaOH/1 M HCl. Two centrifugal filters (Amicon Ultra—0.5 mL, Merck Millipore Ltd.) were each filled with 400 μL of LPEI-l-[N₃:DBCO]-PEG₂₄-HER2-affibody (Compounds 28a and 28b) solution. They were centrifugated one time at 14′000 g for 30 minutes to remove buffer and then three times against 20 mM HEPES, pH 7.2 at 4° C. A concentrated solution of LPEI-l-[N₃:DBCO]-PEG₂₄-HER2-affibody HEPES salt (˜146 μL) was recovered after buffer exchange and supplemented with 170 μL 20 mM HEPES, pH 7.2. A copper assay was performed on the final HEPES salt solution (˜0.3 mL) and a concentration of 1.7 mg/mL total LPEI was measured.

### Example 10

### Synthesis of LPEI-l-[N₃:DBCO]-PEG₃₆-DUPA (Compounds 31a and 31b)

LPEI-l-[N₃:DBCO]-PEG₃₆-DUPA was synthesized as a mixture of regioisomers 31a and 31b according to the schemes below. In a first step, HOOC-PEG₃₆-NH₂ (Compound 32) was coupled to N-succinimidyl 3-maleimidopropionate (Compound 33) by amine formation to produce HOOC-PEG₃₆-MAL (Compound 34). In a next step, HOOC-PEG₃₆-MAL (Compound 34) was coupled to DBCO-NH₂ (Compound 35) by amine formation to produce DBCO-PEG₃₆-MAL (Compound 36). In a next step, DBCO-PEG₃₆-MAL (Compound 36) was coupled to DUPA-Aoc-Phe-Gly-Trp-Trp-Gly-Cys (Compound 12; SEQ ID NO:4) by a Michael addition to produce DBCO-PEG₃₆-DUPA (Compound 37). In a next step, DBCO-PEG₃₆-DUPA (Compound 37) was coupled to LPEI-N₃ by a [2+3]cycloaddition to produce LPEI-l-[N₃:DBCO]-PEG₃₆-DUPA as a mixture of regioisomers 31a and 31b.

### Step 1: Synthesis of HOOC-PEG₃₆-MAL (Compound 34)

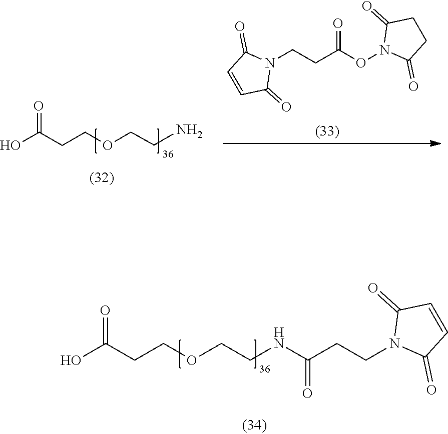

Stock solutions were prepared as follows: HOOC-PEG₃₆-NH₂ (Compound 32) was weighed (364.4 mg, 218 μmol, 1.0 eq) in a 50 mL Falcon tube and 5.0 mL of DCM were added to yield a 44 mM stock solution. N-succinimidyl 3-maleimidopropionate (Compound 33) was weighed (83.0 mg, 312 μmol) in a 5.0 mL Eppendorf tube and 3.0 mL of DCM were added to yield a 104 mM stock solution.

To the HOOC-PEG₃₆-NH₂ containing Falcon tube, DIEA (55.6 μL, 327 μmol, 1.5 eq) and 2.308 mL (240 μmol, 1.1 eq) of N-succinimidyl 3-maleimidopropionate stock solution were added. The reaction mixture was incubated on a Stuart rotator (RT, 15 rpm, protected from light) and monitored by RP-C₈-HPLC. After 30 minutes, all the HOOC-PEG₃₆-NH₂ had reacted. After a total of two hours the reaction mixture (˜7.3 mL) was purified by precipitation: 30 mL of n-hexane were added and the mixture was vortexed for a few seconds and centrifugated (10 min; 4′400 rpm). A yellow oil was recovered and dried overnight (25° C., 10 mbar). 458 mg (crude mass) of a white-yellowish material (crude HOOC-PEG₃₆-MAL; Compound 34) were recovered and analyzed by RP-C8-HPLC; qTOF mass spectrometry (calculated monoisotopic mass: 1′825.02 Da; measured: 1′825.02 Da).

### Step 2: Synthesis of DBCO-PEG₃₆-MAL (Compound 36)

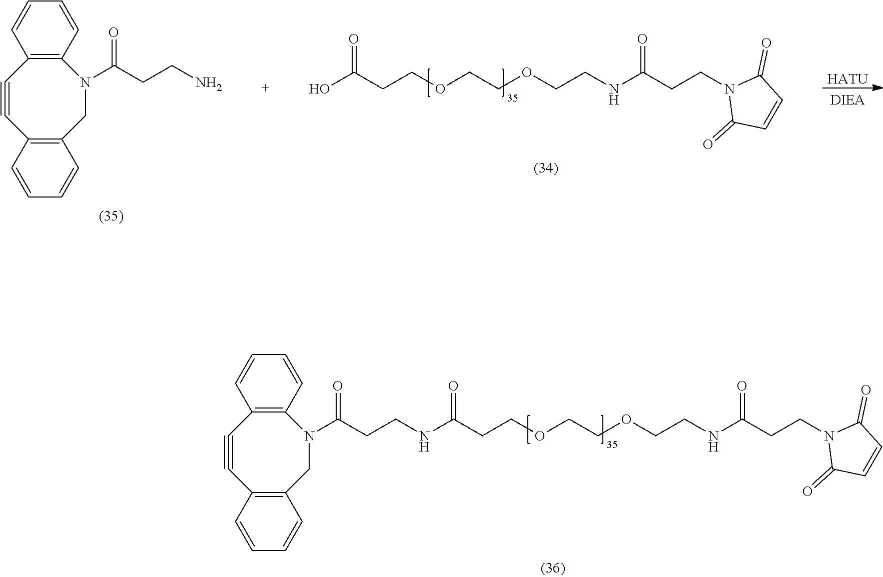

A stock solution of HOOC-PEG₃₆-MAL was prepared by dissolving 458 mg (crude mass) of HOOC-PEG₃₆-MAL (Compound 34) in 4.0 mL DCM. For the stoichiometry calculations, it was assumed that the crude mass was pure HOOC-PEG₃₆-MAL (246 μmol, 1.0 eq). A stock solution of DBCO-NH₂ (Compound 35) was prepared by weighing 84.0 mg of DBCO-NH₂ (246 μmol) in a 5.0 mL Eppendorf tube followed by the addition of 1.0 mL of DMF to yield a 304 mM stock solution. A stock solution of HATU was prepared by weighing 82.5 mg of HATU (217 μmol) in a 5.0 mL Eppendorf. 1.0 mL of DMF were added to yield a 217 mM stock solution.

To the HOOC-PEG₃₆-MAL (Compound 34), 1.0 mL (221 μmol, 0.9 eq) of HATU stock solution were added. The solution was stirred on a Stuart rotator for about one minute. DIEA (75 μL, 442 μmol, 2.0 eq) were added and the solution was stirred for about 3 minutes followed by the addition of DBCO-NH₂ (Compound 35; 728 μL, 221 μmol, 0.9 eq) stock solution. The reaction mixture was incubated on a Stuart rotator (15 rpm, RT, light protected) and was monitored by RP-C₈-HPLC. After one hour of incubation, additional DBCO-NH₂ solution (80 μL, 25 μmol, 0.1 eq) was added to the reaction mixture to ensure complete consumption of HOOC-PEG₃₆-MAL. After 3 hours the reaction mixture (˜5.9 mL) was purified by precipitation. n-Hexane (30 mL) was added on the reaction mixture, vortexed and centrifugated (10 min; 4′400 rpm). The supernatant was discarded and 20 mL of cold diethyl ether were added. The precipitate was recovered and dried overnight in a vacuum-drying oven (25° C., 10 mbar). DBCO-PEG₃₆-MAL (Compound 36), was recovered as a light yellow solid (542 mg) and analysed for purity by RP-C₈-HPLC and qTOF mass spectrometry (calculated monoisotopic mass: 2′083.13 Da; measured: 2′083.14 Da).

### Step 3: Synthesis of DBCO-PEG₃₆-DUPA

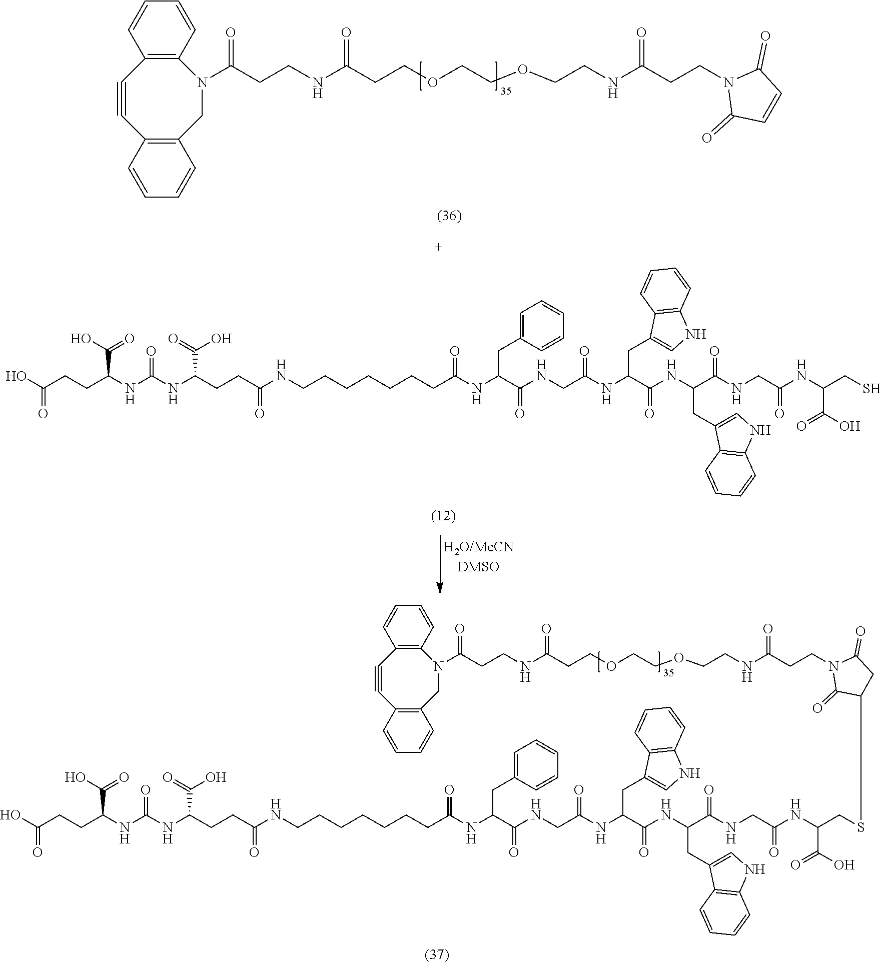

A stock solution of DBCO-PEG₃₆-MAL (Compound 36) was prepared by dissolving 548 mg in a 50 mL Falcon tube and dissolving in 10 mL DMSO (26.3 mM stock solution). A stock 5 solution of DUPA-Aoc-Phe-Gly-Trp-Trp-Gly-Cys (Compound 12; SEQ ID NO.4) was prepared by weighing 318 mg in a 250 mL round-bottom flask equipped with a magnetic stirrer.

Acetate buffer (15 mM, 159 mL, pH 5.2) was added and the mixture was agitated for a few minutes until complete dissolution of Compound 12. The solution was adjusted to pH 5.5 with 350 μL of 5 M NaOH. DBCO-PEG₃₆-MAL stock solution (10 mL, 263 μmol, 1.0 eq) was slowly added to the Compound 12 solution (265 μmol, 1.0 eq,) and the reaction mixture was stirred and protected from light. The reaction was monitored with RP-C8 HPLC. After one hour the excess of Compound 12 was removed by TFF (2 kDa MWCO membrane). The solution (˜169 mL) was ultrafiltered using TFF against 15 mM acetate buffer (pH 4.8). The recovered solution (˜55 mL) was lyophilized for about 48 hours on a freeze-drying device and the lyophilisate was analyzed by RP-C8-HPLC. Residual impurities were removed by precipitation. 500 mg of the lyophilized material were dissolved in 6 mL DMF in a 50 mL Falcon tube. To the slightly turbid solution, cold diethyl ether (30 mL) was added, and a precipitate was formed, collected and washed with cold diethyl ether (30 mL) and dried in a vacuum oven overnight (25° C.; 10 mbar) to give 270 mg DBCO-PEG₃₆-DUPA (Compound 37). qTOF mass spectrometry (calculated monoisotopic mass: 3′280.60 Da; measured: 3′280.64 Da)

### Step 4: Synthesis of LPEI-l-[N₃:DBCO]-PEG₃₆-DUPA

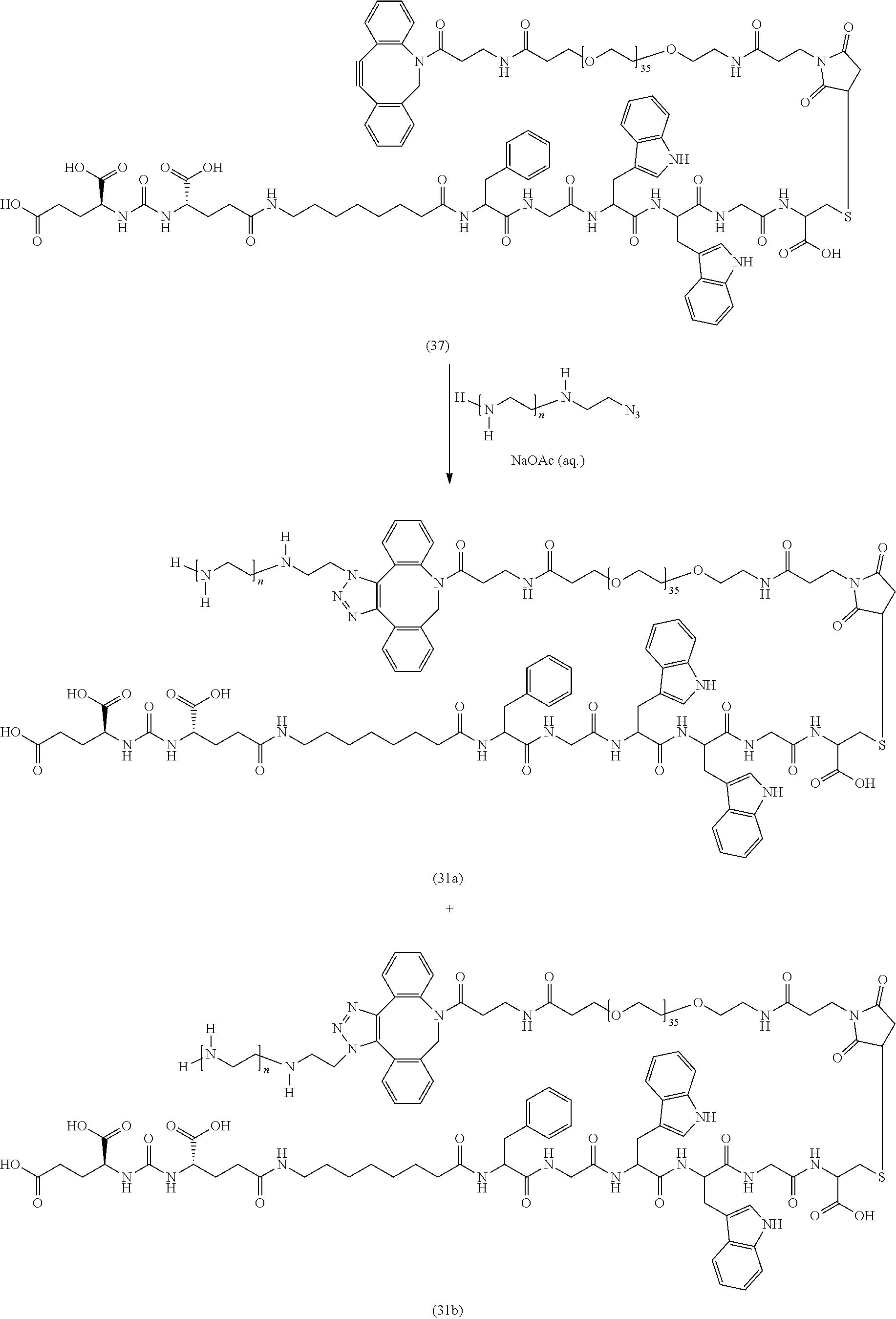

LPEI-N₃ 1013 mg (crude mass) were weighed in a 50 mL Falcon tube and dissolved in 35.0 mL of 50 mM acetate buffer, pH 4.0. The solution was acidified and sonicated for 10 minutes to fully dissolve the LPEI-N₃ and the final pH was adjusted to pH 4.0. A concentration of 22.1 mg/mL in total LPEI amine (1.0 mM) was determined by copper assay (corresponding to a content in LPEI-N₃ of 82% of the crude mass). A stock solution of DBCO-PEG₃₆-DUPA (Compound 37) was prepared by dissolving 219 mg of DBCO-PEG₃₆-DUPA in a 50 mL Falcon tube with 20.0 mL of 50 mM acetate buffer. The pH of the solution was adjusted to pH 4.0 by adding 1 M HCl. The concentration in DBCO was determined by spectrophotometry at 309 nm with Nanodrop One C and was measured at 2.0 mM. DBCO-PEG₃₆-DUPA solution (˜21 mL, 40 μmol) was slowly added to the magnetically stirred solution of the LPEI solution (37 mL, 38 μmol, 1.0 eq). The mixture was stirred for 72 hours at room temperature and protected from light. The reaction mixture (˜60 mL) was supplemented with acetonitrile (10% ACN final volume) and with TFA (1% TFA final volume). The solution turned cloudy but became clear after adjusting the pH to pH 3.5 with 5 M NaOH. Purification was by preparative RP-C₁₈-HPLC. Pooled fractions of LPEI-l-[N₃:DBCO]-PEG₃₆-DUPA were recovered as a mixture of regioisomers 31a and 31b. The fractions were lyophilized to give 830 mg lyophilisate as a TFA salt, 34% weight LPEI content by Cu assay). The pooled fractions containing purified products were analyzed by RP-HPLC, copper assay, and UV spectrophotometry at 280 nm. An 20 μLPEI:DUPA molar ratio of 1:1 was determined.

### Step 5: Preparation of LPEI-l-[N₃:DBCO]-PEG₃₆-DUPA (Compounds 31a and 31b) HEPES Salt

To exchange TFA by HEPES, 421 mg (crude mass) of lyophilized LPEI-l-[N₃:DBCO]-PEG₃₆-DUPA-TFA salt (w_{LPEI}=34%, ˜143 mg in total LPEI) were dissolved in 30 mL 20 mM HEPES pH 7.2 in a 50 mL Falcon tube. The pH was adjusted to pH 6.0 with 11 μL 5 M NaOH and 7 μL 6 M HCl. TFF was performed against 20 mM HEPES pH 7.2 with a total dilution of 10′757×. About 45 mL of LPEI-l-[N3:DBCO]-PEG₃₆-DUPA HEPES salt solution were recovered after TFF. Copper assay and RP-C8-HPLC were performed on the final LPEI-l-[N₃:DBCO]-PEG₃₆-DUPA (Compounds 31a and 31b) HEPES salt solution (˜45 mL) and a concentration of 2.7 mg/mL total LPEI (ratio LPEI/DUPA=1/1.1) was measured. The yield recovery after TFF was calculated to be 85% based on the total LPEI content.

### Step 6: Preparation of LPEI-l-[N3:DBCO]-PEG₃₆-DUPA (Compounds 31 and 31b) acetate Salt

Lyophilized LPEI-l-[N₃:DBCO]-PEG₃₆-DUPA-TFA salt (4.9 mg, w_{LPEI}=34%, ˜1.7 mg in total LPEI) was dissolved in 0.8 mL 50 mM acetate pH 4.3 in a 1.5 mL Eppendorf tube. The pH was adjusted to pH 4.5 with 3.0 μL 5 M NaOH. Two centrifugal filters (Amicon Ultra—0.5 mL, 3 kDa MWCO) were filled with 400 μL of LPEI-l-[N₃:DBCO]-PEG₃₆-DUPA-TFA salt solution each. They were centrifugated one time at 14′000 g for 30 minutes to remove buffer and then 3 times against 400 μL 50 mM acetate, pH 4.3 at 4° C. A concentrated solution of LPEI-l-[N₃:DBCO]-PEG₃₆-DUPA-acetate salt (177 μL) was recovered after buffer exchange and supplemented with 0.45 mL 50 mM acetate, pH 4.3. Copper assay and analytical RP-C₈-HPLC was performed on the LPEI-l-[N₃:DBCO]-PEG₃₆-DUPA (Compounds 31a and 31b)—acetate salt solution (˜0.6 mL) and a concentration of 2.0 mg/mL total LPEI was determined.

### Example 11

### Synthesis of LPEI-l-[N₃:DBCO]-PEG₃₆-[(NH₂)MAL-S]-DUPA (Compounds 38a and 38b)

LPEI-l-[N₃:DBCO]-PEG₃₆-[(NH₂)MAL-S]-DUPA was synthesized as a mixture of regioisomers 38a and 38b according to the schemes below. In the first step, HOOC-PEG₃₆-NH₂ (Compound 32) was condensed with Mal-L-Dap(Boc)-OH (Compound 39) to give HOOC-PEG₃₆-(Boc)-MAL (Compound 40). Compound 40 was subsequently condensed with DBCO-NH₂ (Compound 35) and deprotected to give DBCO-PEG₃₆-(NH₂)-MAL (Compound 41). Compound 41 was reacted with DUPA-Aoc-Phe-Gly-Trp-Trp-Gly-Cys (Compound 12) via Michael Addition and cyclized with LPEI-N₃ to produce compounds 38a and 38b.

### Step 1. Synthesis of HOOC-PEG₃₆-(Boc)-MAL (Compound 40)

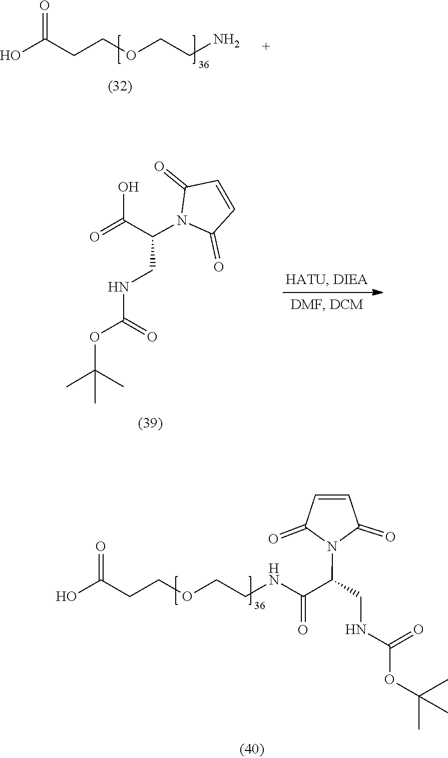

A solution Mal-L-Dap(Boc)-OH (N-α-Maleimido-N—O-t-butyloxycarbonyl-L-2,3-diaminopropionic acid DCHA salt; Compound 39; 50 μmol, 1.1 eq, 294 mM) in DCM (0.17 mL) was mixed with a solution of HATU (45 μmol, 0.9 eq, 217 mM) in DMF (0.207 mL). To the resulting mixture 17 μL of DIEA (100 μmol, 2.0 eq) were added. Finally, HOOC-PEG₃₆-NH₂ (Compound 32, 50 μmol, 1.0 eq, 248 mM) as a solution in DCM (0.20 mL) was added. The reaction mixture was incubated on a Stuart rotator at room temperature and the reaction was monitored by RP-C₈-HPLC. After 1.5 hours, an additional 0.2 eq of Mal-L-Dap(Boc)-OH was added. After a further one and half hours, 5.0 mL of n-hexane were added to induce precipitation and the reaction mixture was centrifuged. The precipitate was washed with 4.5 mL cold diethyl ether. A solid (77 mg) containing crude HOOC-PEG₃₆-(Boc)-MAL (Compound 40) was recovered and analyzed by HPLC-ESI⁺ qTOF mass spectrometry (calculated monoisotopic mass: 1940.08 Da; measured: 1940.10 Da). The crude Compound 40 was used without further purification in the next step.

### Step 2. Synthesis of DBCO-PEG₃₆-(NH₂)-MAL (Compound 41)

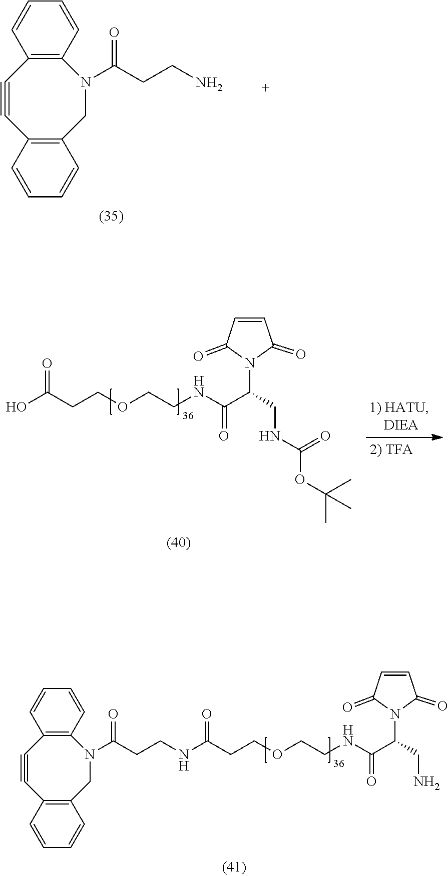

HATU (35 μmol, 0.9 eq, 208 mM) in DMF (169 μL) was added to a solution of HOOC-PEG₃₆-(Boc)-MAL (Compound 40; 39 μmol, 1.0 eq, 98 mM) in DCM (400 mL). The solution was mixed on a Stuart rotator for one minute followed by the addition of DIEA (13 μL, 78 μmol, 2.0 eq) and a solution DBCO-NH₂ (Compound 35; 20 μmol, 0.5 eq, 370 mM) in DMF (53 μL). The reaction mixture was incubated on a Stuart rotator at room temperature and was monitored by RP-C₈-HPLC. At 20 minutes into reaction, an additional amount of DBCO-NH₂ (8 μmol, 0.2 eq) in DMF (22 μL) was added. After a total of 45 min, 4.5 mL cold diethyl ether were added. The precipitate was further washed with 4.5 mL cold diethyl ether. Crude DBCO-PEG₃₆-(Boc)-MAL was isolated as a yellow solid (92 mg) and analyzed by HPLC-ESI⁺ qTOF MS (calculated monoisotopic mass: 2198.20 Da; measured: 2198.20 Da) and dissolved without purification in 2.7 mL DCM and 40 μL TFA.

The Boc group deprotection of DBCO-PEG₃₆-(Boc)-MAL was monitored by RP-C₈-HPLC. Upon completion, n-hexane (2.5 mL) was added and the precipitate was washed with 4.5 mL cold diethyl ether. The recovered solid material (DBCO-PEG₃₆-(NH₂)-MAL; Compound 41) was analyzed by HPLC-ESI⁺ qTOF mass spectrometry (calculated monoisotopic mass: 2098.14 Da; measured: 2098.14 Da).

### Step 3. Synthesis of DBCO-PEG₃₆-[(NH₂)MAL-S]-DUPA (Compound 42)

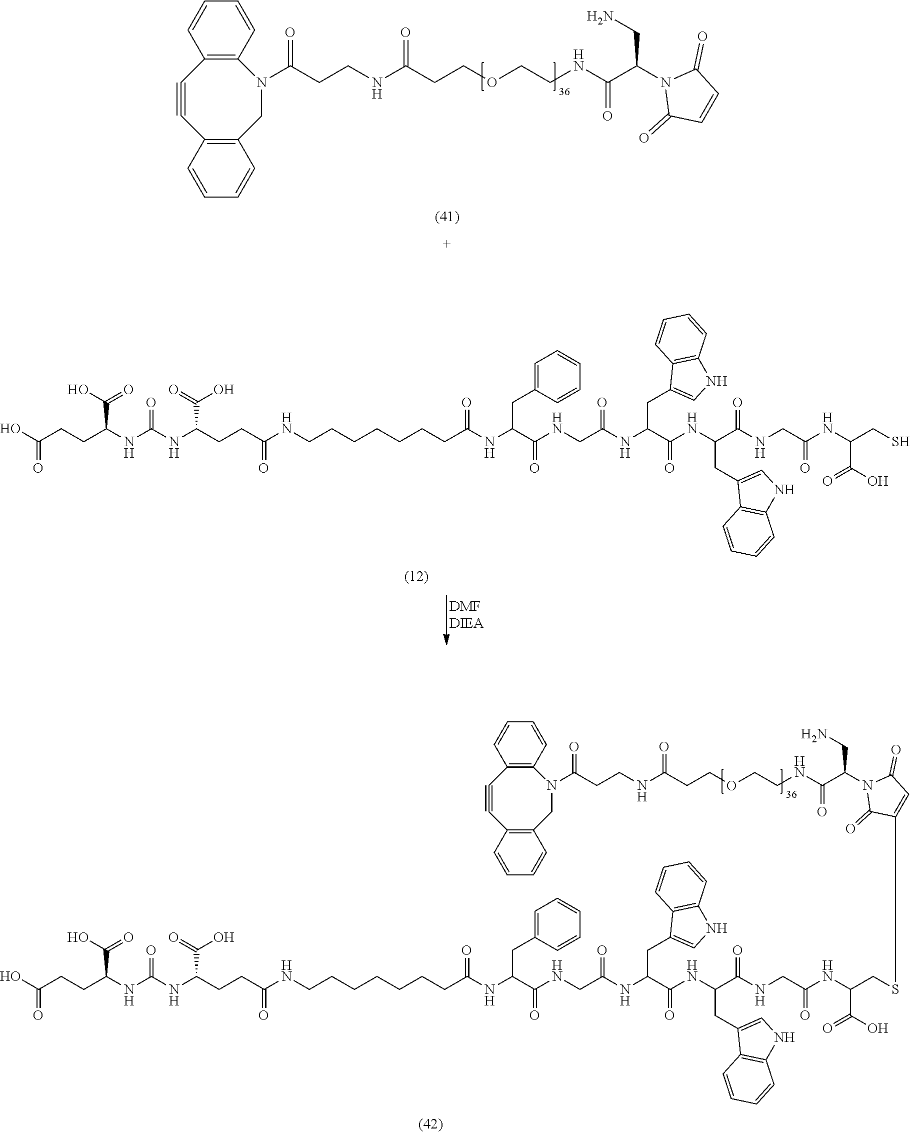

A solution of DUPA-Aoc-Phe-Gly-Trp-Trp-Gly-Cys (Compound 12; SEQ ID NO:4) (20 μmol, 0.5 eq, 142 mM) in DMF (141 μL) was added to 400 μL of a solution of DBCO-PEG₃₆-(NH₂)-MAL (Compound 41; 39 μmol, 1.0 eq, 98 mM) in DMF and 10 μL of DIEA (59 μmol, 3.0 eq). The reaction mixture was incubated on a Stuart rotator at room temperature and monitored by RP-C8-HPLC. After one hour, cold diethyl ether (4.5 mL) was added and the product precipitated. The precipitate was washed with 4.5 mL cold diethyl ether, dissolved in 1.0 mL DMSO and supplemented with a mixture of 1% TFA/H₂O: 1% TFA ACN (14 mL 9:1 v/v). The pH was adjusted to 6.0 to ensure that the solution was clear. The solution of DBCO-PEG₃₆-[(NH₂)MAL-S]-DUPA (Compound 42) was purified using RP-C₁₈ preparative HPLC and the pooled fractions were lyophilized. The lyophilisate was analyzed by RP-HPLC-ELSD and RP-HPLC-ESI⁺ qTOF mass spectrometry (DBCO-PEG₃₆-[(NH₂)MAL-S]-DUPA calculated monoisotopic mass: 3313.64 Da (maleimide ring opened); measured: 3313.66 Da).

### Step 4. Synthesis of LPEI-l-[N₃:DBCO]-PEG₃₆-[(NH₂)MAL-S]-DUPA (Compounds 38a and 38b)

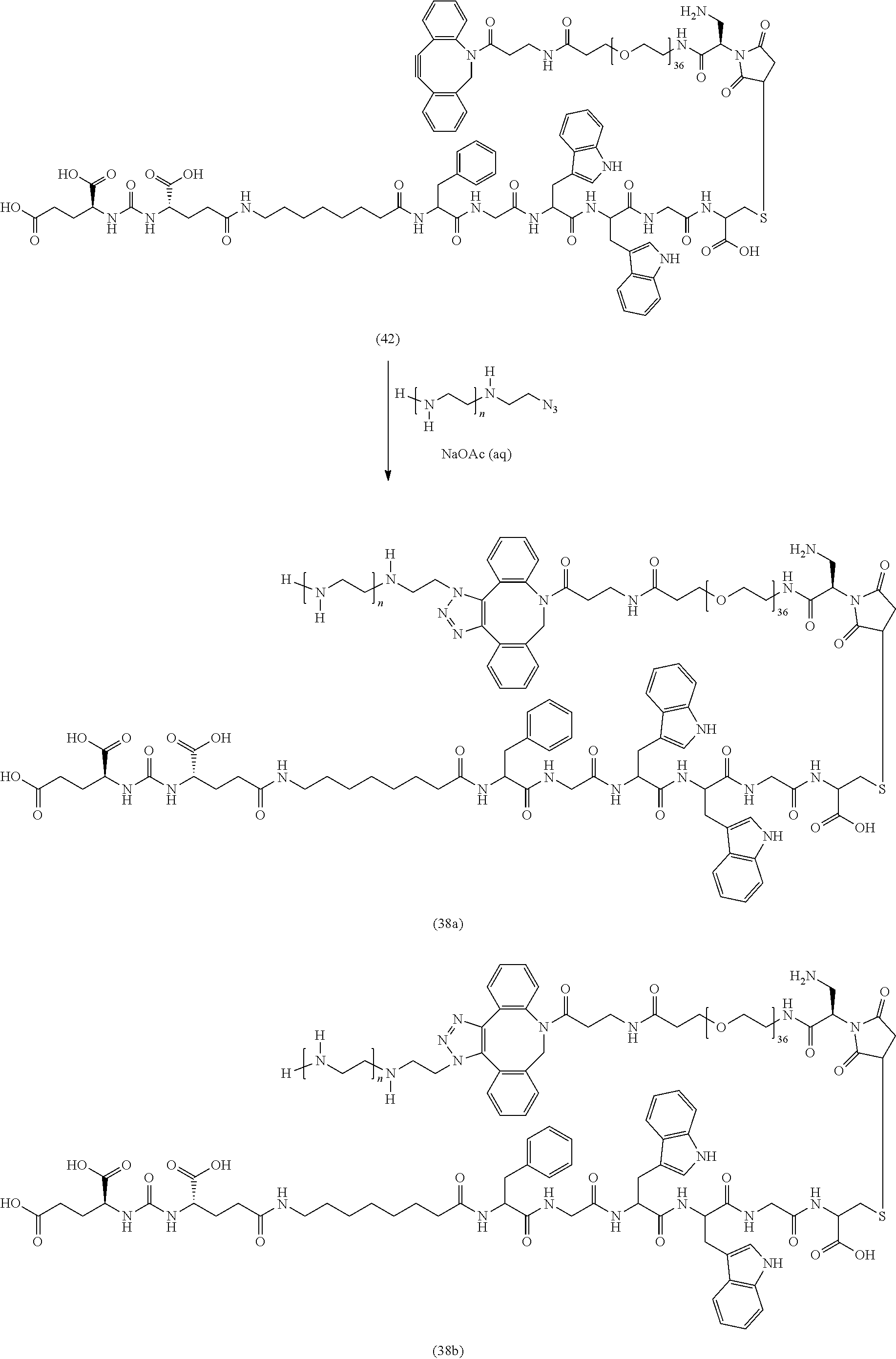

LPEI-N₃ solution (2.3 mL, 2.3 μmol, 1.5 eq, 1.0 mM) in 50 mM acetate buffer pH 4.0 was slowly added to 4.0 mL solution of DBCO-PEG₃₆-[(N₂)MAL-S]-DUPA (Compound 42; 1.5 μmol, 1.0 eq, 0.37 mM). After 70 hours, the reaction mixture was supplemented with 0.78 mL acetonitrile and 78 μL TFA. LPEI-l-[N₃:DBCO]-PEG₃₆-[(NH₂)MAL-S]-DUPA was isolated as a mixture of regioisomers 38a and 38b using RP-C₁a preparative HPLC. Pooled fractions were lyophilized to give 38 mg of a fluffy white solid which was characterized by RP-C₈-HPLC, copper assay and spectrophotometry at 280 nm for determination of the DUPA content. The lyophilisate had a weight percentage in LPEI of 32% w/w and a LPEI to DUPA ratio of 1/1.1.

### Step 5. Preparation of LPEI-l-[N₃:DBCO]-PEG₃₆-[(NH₂)MAL-S]-DUPA (Compounds 38a and 38b) HEPES Salt

LPEI-l-[N₃:DBCO]-PEG₃₆-[(NH₂)MAL-S]-DUPA (Compounds 38a and 38b) TFA salt (21.9 mg, w_{LPEI}=32%, 7.0 mg in total LPEI) were dissolved in 1.2 mL 20 mM HEPES pH 7.5. Three centrifugal filters (Amicon Ultra-0.5 mL, 10 kDa MWCO) were filled with 400 μL of LPEI-l-[N₃:DBCO]-PEG₃₆-[(NH₂)-MAL-S]-DUPA solution each, centrifuged one time at 14′000 g for 30 minutes and then three times after addition of 400 uL 20 mM HEPES, pH 7.2. Approximately 261 μL of LPEI-l-[N₃:DBCO]-PEG₃₆-[(NH₂)MAL-S]-DUPA-HEPES salt solution were recovered and supplemented with 2.4 mL 20 mM HEPES, pH 7.2. The concentration of the solution was determined by copper assay to be 2.2 mg/mL in total LPEI.

### Example 12

### Synthesis of LPEI-l-[N₃:BCN]-PEG₃₆-DUPA (Compound 43)

LPEI-l-[N₃:BCN]-PEG₃₆-DUPA (Compound 43) was synthesized according to the schemes below. Endo-BCN-PEG₃₆-MAL (Compound 45) was prepared by condensing HOOC-PEG₃₆-MAL (Compound 34) with endo-BCN-PEG₂-NH₂ (Compound 44). In a next step, Compound 45 was condensed with Compound 12, and the resulting endo-BCN-PEG₃₆-[MAL-S]-DUPA (Compound 46) was reacted with LPEI-N₃ to give Compound 43.

### Step 1. Synthesis of endo-BCN-PEG₃₆-MAL (Compound 45)

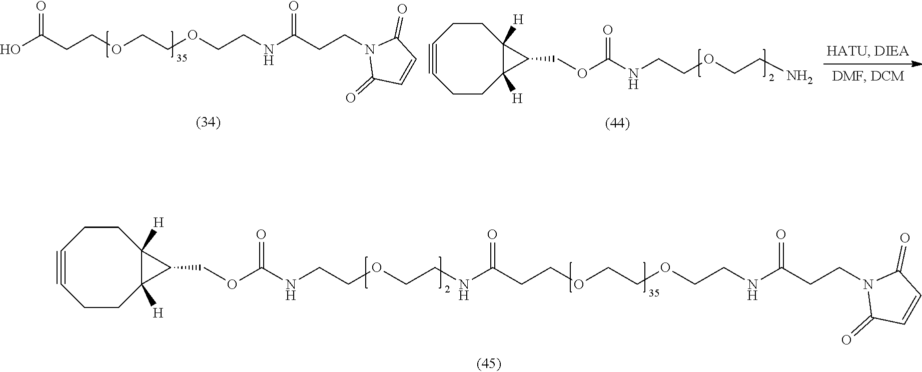

A solution of HATU (20 μmol, 0.9 eq, 123 mM) solution (165 μL) was added to a solution of HOOC-PEG₃6-MAL (Compound 34; see Example 10; 23 μmol, 1.0 eq, 58 mM) in DCM (400 μL) and DIEA (7.7 μL, 45 μmol, 2.0 eq). To the reaction mixture was added endo-BCN-PEG₂-NH₂ (Compound 44; 18 μmol, 0.8 eq, 145 mM) as a solution in DCM (124 μL) and the reaction was monitored by RP-C₈-HPLC. Further amounts of endo-BCN-PEG₂-NH₂ (2×0.2 eq) were added at 20 min intervals. After an additional one hour, n-hexane (4.5 mL) was added to the reaction mixture. The resulting precipitate was separated by centrifugation and washed with 4.5 mL cold diethyl ether and dried under vacuum. Crude endo-BCN-PEG₃₆-MAL (Compound 45; 61 mg) was isolated and analysed by RP-C₈-HPLC coupled with ESI⁺-qTOF mass spectrometry (Calculated monoisotopic mass: 2′131.21 Da; measured: 2′131.22 Da) and used in the next step without further purification.

### Step 2. Synthesis of endo-BCN-PEG₃₆-[MAL-S]-DUPA (Compound 46)

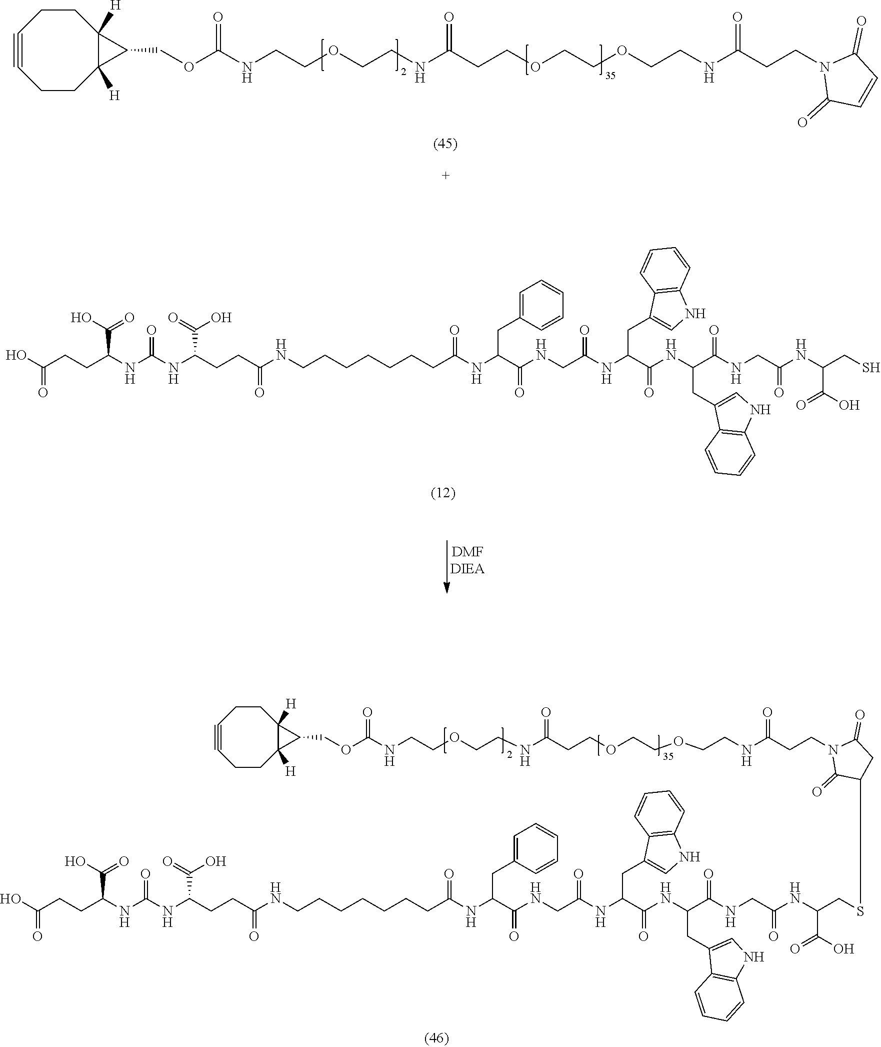

A solution of DUPA-Aoc-Phe-Gly-Trp-Trp-Gly-Cys (Compound 12; SEQ ID NO:4) (21 μmol, 1.1 eq) in DMF (239 μL) was slowly added to a mixture containing endo-BCN-PEG₃₆-MAL (Compound 45; 400 μmol, 1.0 eq, 48 mM) and DIEA (7 μL, 42 μmol, 2.0 eq) in DMF. After one hour, cold diethyl ether (4.5 mL) was added. The precipitated solid was filtered, washed with cold diethyl ether, and dried to give 70 mg of endo-BCN-PEG₃₆-[MAL-S]-DUPA (Compound 46). A sample was analyzed by HPLC ESI⁺ qTOF mass spectrometry (endo-BCN-PEG₃₆-DUPA: calculated monoisotopic mass: 3328.69 Da; measured: 3328.72 Da).

### Step 3. Synthesis of LPEI-l-[N₃:BCN]-PEG₃₆-DUPA (Compound 43)

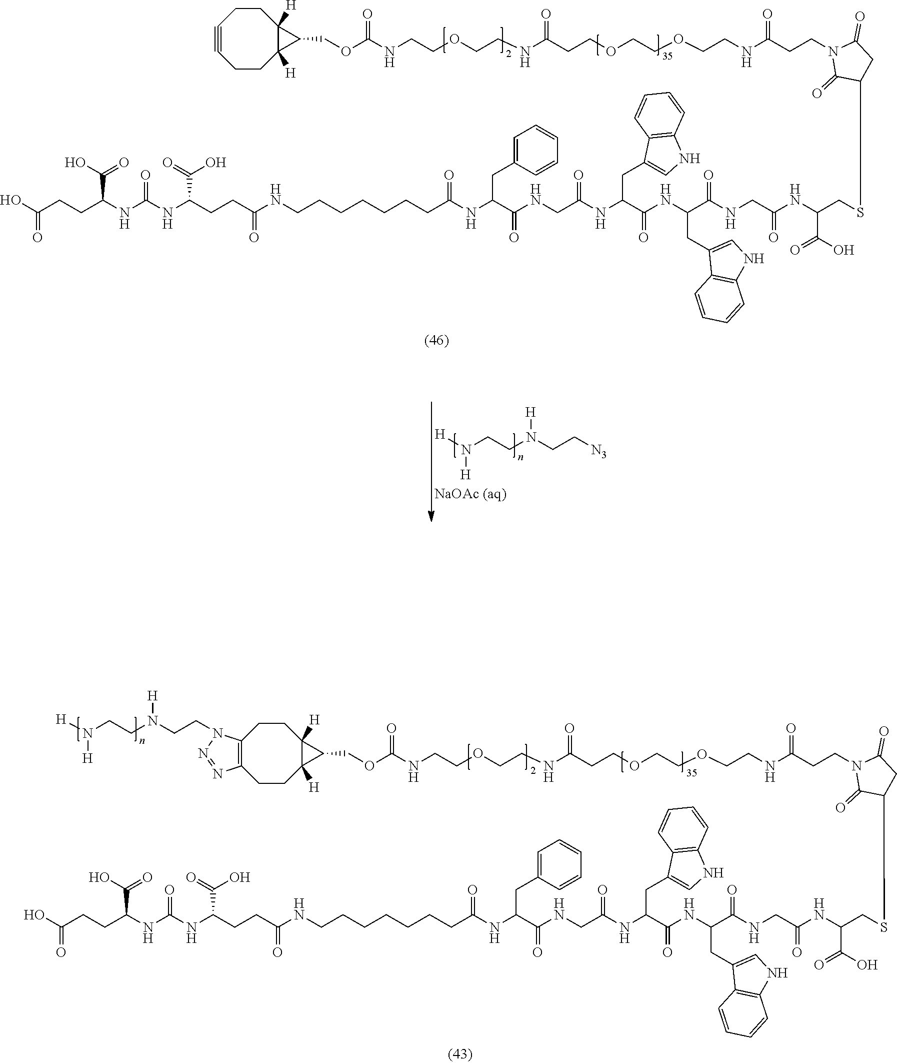

endo-BCN-PEG₃₆-[MAL-S]-DUPA (Compound 46; 3.8 μmol, 1.5 mM, 1.0 eq) in acetate buffer (50 mM, 2.5 mL, pH 4.0) was slowly added to a solution of LPEI-N₃ (4.1 μmol, 1.1 eq, 22 mg/mL) in acetate buffer (50 mM, 4.2 mL, pH 4.0). The mixture was shaken for about 70 hrs at room temperature on a Stuart rotator and protected from light. To the reaction mixture were added 3.0 mL 50 mM acetate buffer, pH 4.0, followed by acetonitrile (1.0 mL) and TFA (100 μL). The resultant mixture was filtered (0.45 μm PA membrane) and purified using RP-C₁₈ preparative chromatography. Pooled fractions containing LPEI-l-[N₃:BCN]-PEG₃₆-DUPA (Compound 43) were lyophilized to give 61 mg lyophilized product and characterized by analytical RP-C₈ HPLC, copper assay and spectrophotometry at 280 nm for determination of the DUPA content. The product was found to have a weight percentage in LPEI of 31% w/w as determined by Cu assay.

Step 4. Preparation of LPEI-l-[N₃:BCN]-PEG₃₆-DUPA (Compound 43) HEPES salt 24.8 mg of LPEI-l-[N₃:BCN]-PEG₃₆-DUPA (Compound 43) TFA salt (w_{LPEI}=31%, 7.7 mg in total LPEI) were dissolved in 1.2 mL 20 mM HEPES pH 7.2. The pH was adjusted to pH 7.3. Three centrifugal filters (Amicon Ultra-0.5 mL, 10 kDa MWCO) were filled with 400 μL of LPEI-l-[N₃:BCN]-PEG₃₆-DUPA solution each. They were centrifugated one time at 14′000 g for 30 minutes and then three times after addition of 400 μL 20 mM HEPES, pH 7.2 at 20° C. About 263 μL of the concentrated solution of LPEI-l-[N₃:BCN]-PEG₃₆-DUPA HEPES salt were recovered after buffer exchange and were supplemented with 2.4 mL 20 mM HEPES, pH 7.2. The concentration of the solution was determined by copper assay to be 2.3 mg/mL in total LPEI.

### Step 5. Preparation of LPEI-l-[N₃:BCN]-PEG₃₆-DUPA (Compound 43) Acetate Salt

5.5 mg of LPEI-l-[N₃:BCN]-PEG₃₆-DUPA (Compound 43) TFA salt (w_{LPEI}=31%, ˜1.7 mg in total LPEI) were dissolved in 0.8 mL 50 mM acetate, pH 4.0. Two centrifugal filters (Amicon Ultra—0.5 mL, 3 kDa MWCO) were filled with 400 μL of LPEI-l-[N₃:BCN]-PEG₃₆-DUPA solution each. They were centrifuged one time at 14′000 g for 30 minutes and then three times after addition of 400 μL 50 mM acetate, pH 4.3. About 144 μL of LPEI-l-[N₃:BCN]-PEG₃₆-DUPA acetate salt solution were recovered and supplemented with 0.6 mL 50 mM acetate, pH 4.3. The concentration of the solution was determined by copper assay to be 2.2 mg/mL in total LPEI.

### Example 13

### Synthesis of LPEI-l-[N₃:SCO]-PEG₃₆-DUPA (Compounds 47a and 47b)

LPEI-l-[N₃:SCO]-PEG₃₆-DUPA was synthesized as a mixture of regioisomers 47a and 47b according to the schemes below. SCO-PEG₃₆-MAL (Compound 49) was prepared by condensing HOOC-PEG₃₆-MAL (Compound 34) with SCO-PEG₃-NH₂ (Compound 48). Compound 49 was reacted with Compound 12 via Michael Addition, and the resulting SCO-PEG₃₆-DUPA (Compound 50) was reacted with LPEI-N₃ to synthesize Compounds 47a and 47b.

### Step 1. Synthesis of SCO-PEG₃₆-MAL (Compound 49)

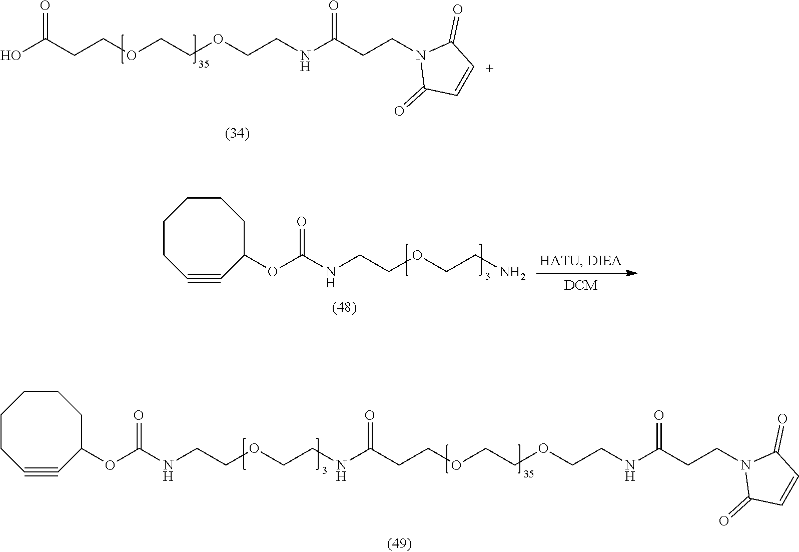

A solution of HATU (25 μmol, 0.9 eq, 147 mM) in DMF (69 μL) was added to HOOC-PEG₃₆-MAL (Compound 34; 28 μmol, 1.0 eq, 70 mM) in DCM followed by DIEA (9.6 μL, 56 μmol, 2.0 eq). To the reaction mixture was added a solution of SCO-PEG₃-NH₂ (Compound 48; 22 μmol, 0.8 eq, 137 mM) in DCM (166 μL). The reaction was placed on a Stuart rotator and reaction progress was monitored by RP-C₈-HPLC. After 10 min, HATU (0.1 eq) and two additional lots of SCO-PEG₃-NH₂ (0.2 eq and 0.1 eq) were added to the reaction mixture. After a total of 1 hr 30 min, 4.5 mL of n-hexane were added. The precipitated solid was washed with 4.5 mL cold diethyl ether and dried. SCO-PEG₃₆-MAL (Compound 49) was isolated as a yellow solid (69 mg) and characterized by analytical RP-C₈-HPLC and ESI⁺ qTOF mass spectrometry (calculated monoisotopic mass: 2149.2 Da; measured: 2149.2 Da).

### Step 2. Synthesis of SCO-PEG₃₆-[MAL-S]-DUPA (Compound 31)

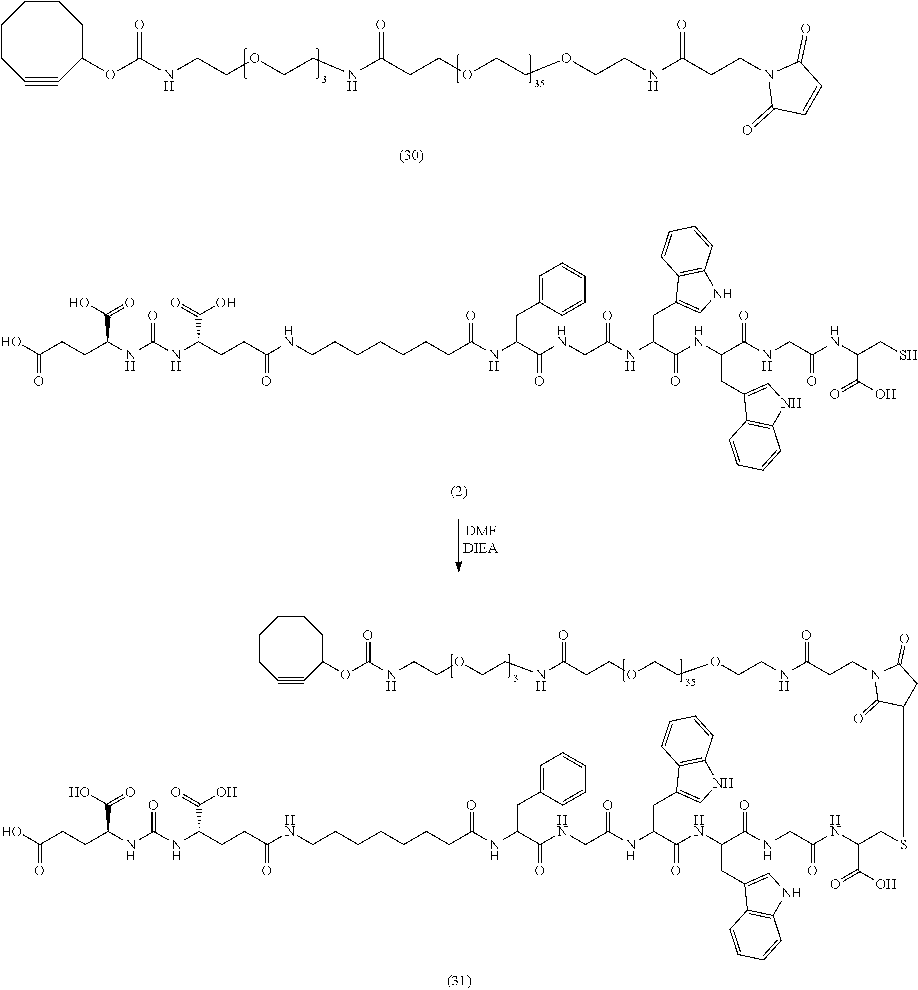

A solution of DUPA-Aoc-Phe-Gly-Trp-Trp-Gly-Cys (Compound 12; SEQ ID NO:4) (15 μmol, 0.5 eq, 100 mM) in DMF (150 μL) and DIEA (10 μL, 62 μmol, 2.0 eq) were added to a solution of SCO-PEG₃₆-MAL (Compound 49; 31 μmol, 1 eq, 78 mM) in DMF. The reaction mixture was placed on a Stuart rotator. After 10 min a further amount of Compound 12 (30 μL, 3 μmol, 0.1 eq) was added. After one hour cold diethyl ether was added and the resultant precipitate was washed with 4.5 mL of cold diethyl ether and dried. The solid (98 mg) was resuspended in 0.5 mL DMSO and diluted with 7.5 mL H₂O (+1% TFA)/CAN (+1% TFA) (9:1 v/v) and purified by prepRP-C₁₈-HPLC. Pooled fractions of SCO-PEG₃₆-DUPA (Compound 50) were lyophilized and analyzed by HPLC-ESI⁺ qTOF mass spectrometry (SCO-PEG₃₆-DUPA calculated monoisotopic mass: 3346.70 Da; measured: 3346.71 Da).

### Step 3: Synthesis of LPEI-l-[N₃: SCO]-PEG₃₆-[MAL-S]-DUPA (Compounds 28a and 28b)

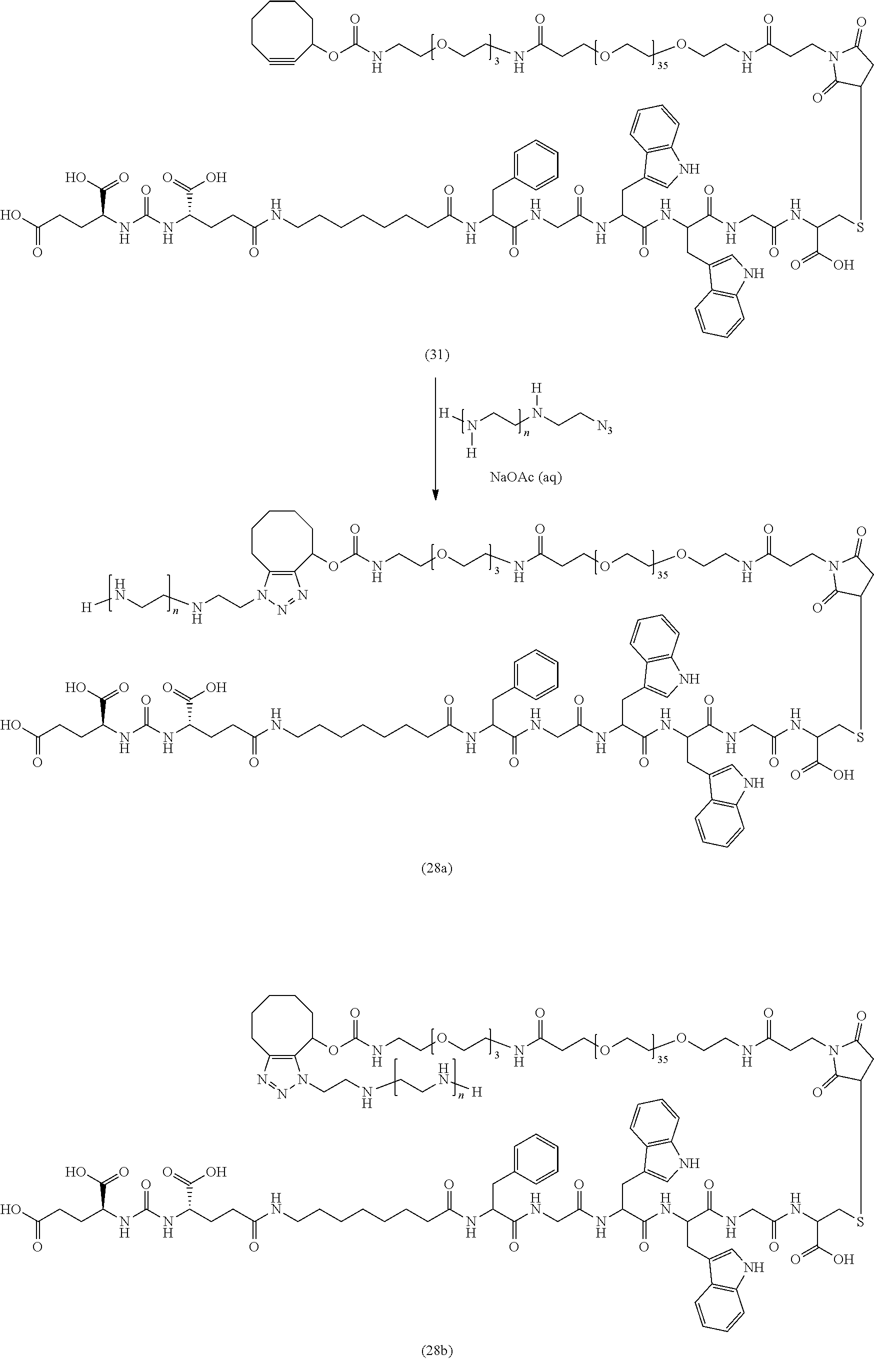

LPEI-N₃ solution (4.2 mL, 5 μmol, 1.0 eq) in 50 mM acetate buffer pH 4.0 was slowly added to 5.0 mL of a SCO-PEG₃₆-DUPA (Compound 50) solution (5 μmol, 1.0 eq, 1 mM) in 50 mM acetate buffer pH 4.0. The mixture was incubated for about 90 hours at room temperature on a Stuart rotator and protected from light. Acetonitrile (1 mL) and TFA (100 μL) were added to the reaction mixture for preparative RP-Cis HPLC purification. Pooled fractions were lyophilized to give 66 mg LPEI-l-[N₃:SCO]-PEG₃₆-DUPA as a mixture of regioisomers 47a and 47b. The lyophilized solid was characterized by analytical RP-C₈ HPLC, copper assay and spectrophotometry at 280 nm. A weight percentage in LPEI of 26% w/w was determined by copper assay for the lyophilized solid.

### Step 4. Preparation of LPEI-l-[N₃:SCO]-PEG₃₆-DUPA (Compounds 47a and 47b) HEPES Salt

23.2 mg of LPEI-l-[N₃:SCO]-PEG₃₆-DUPA (Compounds 47a and 47b) TFA salt (w_{LPEI}=26%, 6.0 mg in total LPEI) were dissolved in 1.2 mL 20 mM HEPES pH 7.4. Three centrifugal filters (Amicon Ultra—0.5 mL, 10 kDa MWCO) were filled with 400 μL of LPEI-l-[N₃:SCO]-PEG₃₆-DUPA solution each, centrifuged one time at 14′000 g for 30 minutes and then three times after addition of 400 μL 20 mM HEPES, pH 7.2. About 276 μL of LPEI-l-[N₃:SCO]-PEG₃₆-DUPA HEPES salt solution were recovered and supplemented with 2.4 mL 20 mM HEPES, pH 7.2. The concentration of the solution was determined by copper assay to be 2.1 mg/mL in total LPEI.

### Example 14

### Synthesis of LPEI-l-[N₃:DBCO]CONH-PEG₃₆-DUPA Compounds 51a and 51b)

LPEI-l-[N₃:DBCO]CONH-PEG₃₆-DUPA was synthesized as a mixture of regioisomers 51a and 51b according to the schemes below. DBCO-PEG₃₆-[CONH]-MAL (Compound 54) was prepared by condensing DBCO-PEG₃₆-TFP (Compound 52) with NH2-MAL (Compound 53). The resulting DBCO-PEG₃₆-[CONH]-MAL (Compound 54) was condensed with Compound 12 and reacted with LPEI-N₃ to give Compounds 51a and 51b.

### Step 1. Synthesis of DBCO-PEG₃₆-[CONH]-MAL (Compound 54)

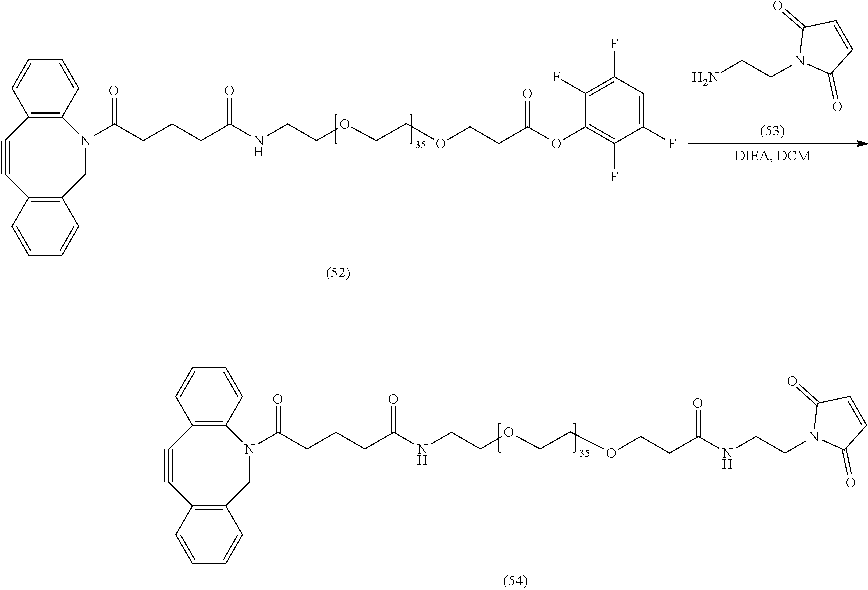

A solution of DBCO-PEG₃₆-TFP (Compound 52; 24 μmol, 1.0 eq, 60 mM) in DCM (0.40 mL) was mixed with a solution of NH₂-MAL (Compound 53; 26 μmol, 1.1 eq, 480 mM) in DMF (55 μL) and DIEA (8 μL, 48 μmol, 2.0 eq). The reaction mixture was incubated on a Stuart rotator at room temperature and the reaction was monitored by RP-C₈-HPLC. After two hours, n-hexane (4.5 mL) was added and the product was precipitated. The precipitate was washed with 4.5 mL cold diethyl ether. Recovered material was analyzed by RP-HPLC-ESI⁺ qTOF mass spectrometry. The solid contained DBCO-PEG₃₆-[CONH]-MAL (Compound 54; calculated monoisotopic mass: 2097.15 Da; measured: 2097.16 Da).

### Step 2. Synthesis of DBCO-PEG₃₆-[CONH]-DUPA (Compound 55)

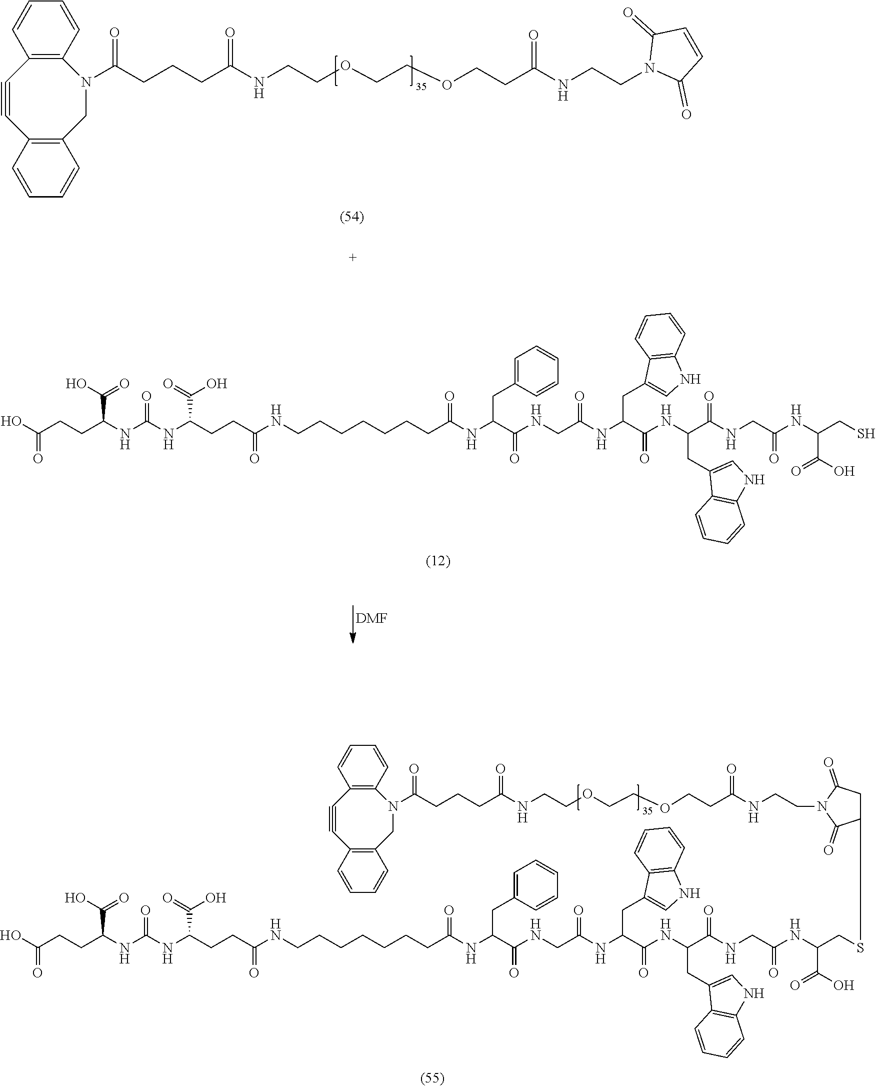

A solution of DBCO-PEG₃₆-[CONH]-MAL (Compound 54; 24 μmol, 1.0 eq, 120 mM) in DMF (0.20 mL) was mixed with a DUPA-Aoc-Phe-Gly-Trp-Trp-Gly-Cys (Compound 12; 5 SEQ ID NO.4) (17 μmol, 0.7 eq, 123 mM) in DMF (137 μL). The reaction mixture was incubated on a Stuart rotator at room temperature and protected from light. After 15 min, an additional amount of DUPA-Aoc-Phe-Gly-Trp-Trp-Gly-Cys (39 μL, 5 μmol, 0.2 eq) was added. At 40 min into reaction, an additional amount of DUPA-Aoc-Phe-Gly-Trp-Trp-Gly-Cys (14 μL, 1.7 μmol, 0.07 eq) was added. After a further one hour mixing, cold diethyl ether (4.5 mL) was added. The precipitate was washed with cold diethyl ether (4.5 mL). The precipitate was dissolved in DMSO (0.5 mL) and was supplemented with H₂O (6.75 mL) and acetonitrile (0.75 mL). DBCO-PEG₃₆-[CONH]-DUPA (Compound 55) was isolated following RP-Cis preparative HPLC and lyophilization of pooled fractions. The lyophilisate was analyzed by RP-HPLC-ELSD and RP-HPLC-ESI⁺ qTOF mass spectrometry (Solid DBCO-PEG₃₆-[CONH]-DUPA (Compound 55; 36 mg) calculated monoisotopic mass: 3294.64 Da; measured: 3294.65 Da).

### Step 3. Synthesis of LPEI-l-[N₃:DBCO]CONH-PEG₃₆-DUPA (Compounds 51a and 51b)

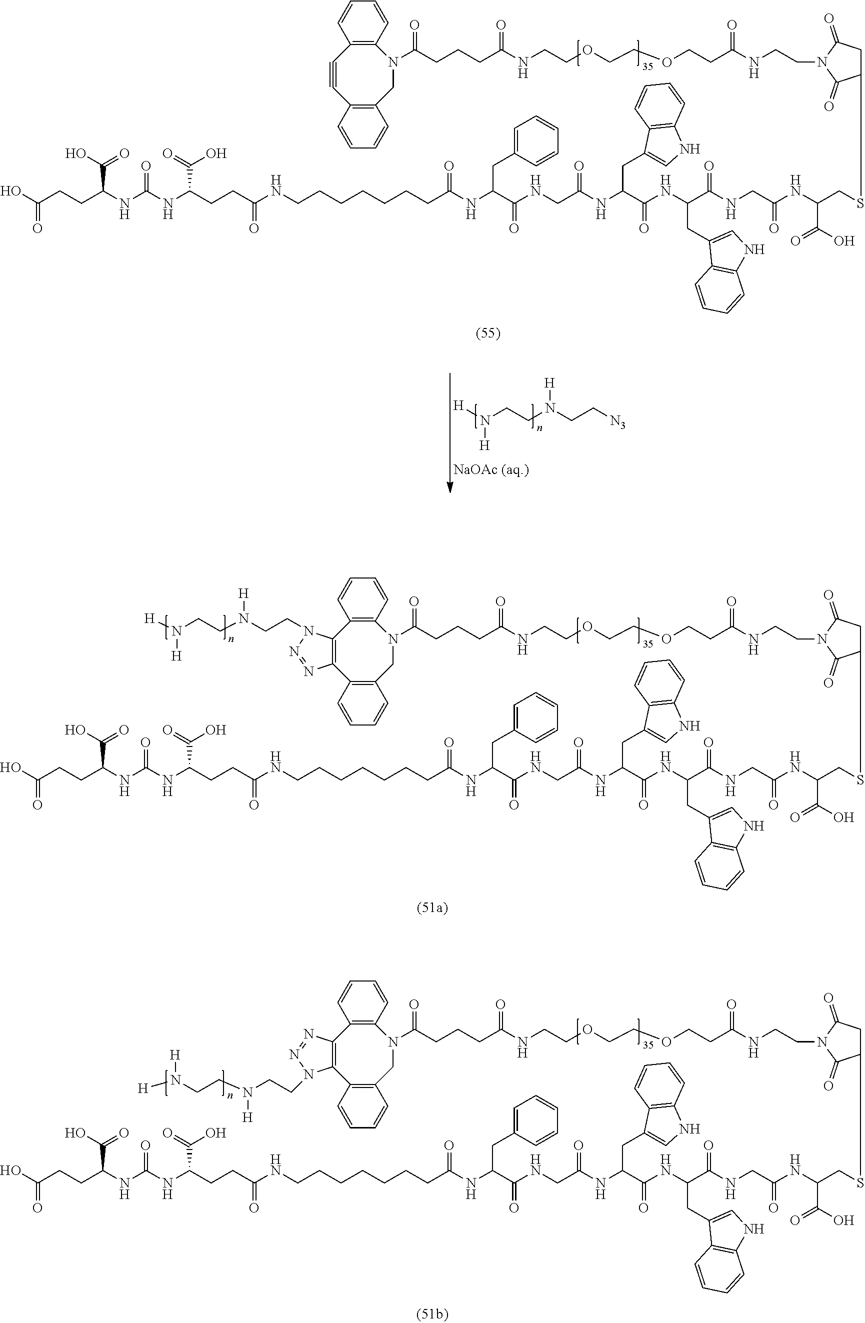

LPEI-N₃ solution (4.2 mL, 5 μmol, 1.0 eq, 1.2 mM) in 50 mM acetate buffer pH 4.0 was slowly added to 2.4 mL of a solution of DBCO-PEG₃₆-[CONH]-DUPA (Compound 55; 5 μmol, 1.0 eq, 2.0 mM). The mixture was incubated at room temperature on a Stuart rotator and monitored by RP-C₈-HPLC. After 70 hours, the reaction mixture was supplemented with acetonitrile (0.73 mL) and TFA (74 μL) and isolated using RP-C₁₈ preparative HPLC. The pooled fractions were lyophilized to give LPEI-l-[N₃:DBCO]-PEG₃₆-[CONH]-DUPA (87 mg) as a mixture of regioisomers 51a and 51b and as a fluffy white solid. The lyophilizate was characterized by RP-C₈-HPLC, copper assay and spectrophotometry at 280 nm for determination of the DUPA content. The lyophilisate had a weight percentage in LPEI of 30% w/w and a LPEI to DUPA ratio of 1/1.1.

### Step 4. Preparation of LPEI-l-[N₃:DBCO]-PEG₃₆-[CONH]-DUPA (Compounds 51a and 51b) HEPES Salt

LPEI-l-[N₃:DBCO]-PEG₃₆-[CONH]-DUPA (Compounds 51a and 51 b) TFA salt (20.8 mg, w_{LPEI}=30%, 6.2 mg in total LPEI) was dissolved in 1.2 mL 20 mM HEPES pH 7.2. Three centrifugal filters (Amicon Ultra—0.5 mL, 10 kDa MWCO) were filled with 400 μL of LPEI-l-[N₃:DBCO]-PEG₃₆-[CONH]-DUPA solution each, centrifugated one time at 14000 g for 30 minutes and then three times after addition of 400 μL 20 mM HEPES, pH 7.2. About 246 μL of LPEI-l-[N₃:DBCO]-PEG₃₆-[CONH]-DUPA-HEPES salt solution were recovered and supplemented with 2.4 mL 20 mM HEPES, pH 7.2. The concentration of the solution was determined by copper assay to be 2.1 mg/mL in total LPEId.

### Example 15

### Synthesis of LPEI-l-[N₃:DBCO]-PEG₃₆-[S-MAL]-DUPA (Compounds 56a and 56b)

LPEI-l-[N₃:DBCO]-PEG₃₆-[S-MAL]-DUPA was prepared as a mixture of regioisomers 56a and 56b according to the schemes below. DBCO-PEG₃₆-SH (Compound 59) was prepared by condensing DBCO-NH₂ (Compound 35) with NHS-PEG₃₆-OPSS (Compound 57) and subsequent reduction. Compound 59 was then condensed with DUPA-MAL (Compound 60) and reacted with LPEI-N₃ to give Compounds 56a and 56b.

### Step 1. Synthesis of DBCO-PEG₃₆-SPDP (Compound 57)

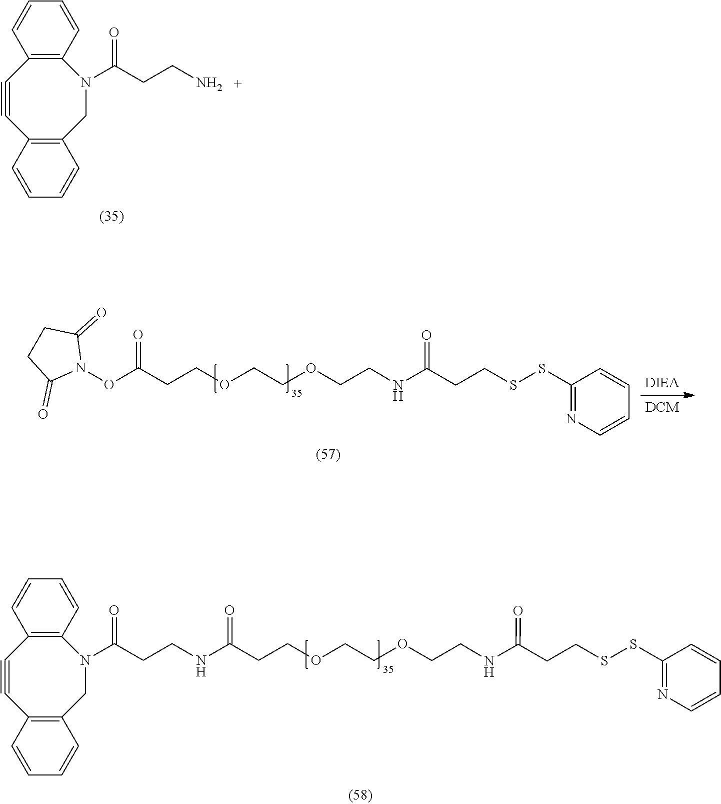

A solution of NHS-PEG₃₆-OPSS (Compound 57; 49 μmol, 1.0 eq, 123 mM) in DCM (0.40 mL) was mixed with a solution containing DBCO-NH₂ (Compound 35; 54 μmol, 1.1 eq, 357 mM and DIEA (17 μL, 100 μmol, 2.0 eq)) in DMF (151 μL). The reaction mixture was incubated on a Stuart rotator at room temperature and the reaction was monitored by RP-C₈-HPLC. After 15 min, an additional amount of DBCO-NH₂ (5 μmol, 0.1 eq, 357 mM) was added. After a total of 30 minutes, 4.5 mL of n-hexane were added. The resulting precipitate was filtered, centrifuged, and washed with 4.5 mL cold diethyl ether. Solid DBCO-PEG₃₆-OPSS (Compound 58) was recovered and analyzed by HPLC-ESI+ qTOF mass spectrometry (calculated monoisotopic mass: 2129.10 Da; measured: 2129.12 Da) and used in the next step without further purification.

### Step 2. Synthesis of DBCO-PEG₃₆-SH (Compound 59)

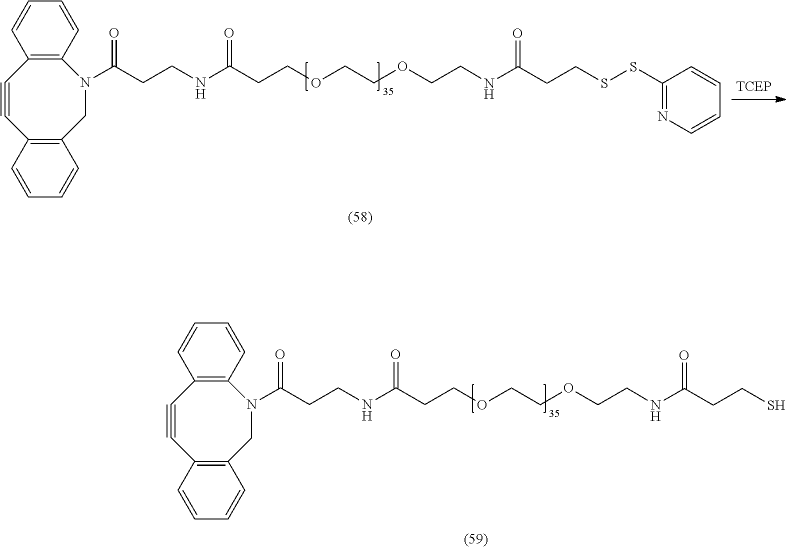

A solution of DBCO-PEG₃₆-OPSS (Compound 58; 4.8 μmol, 1.0 eq, 12 mM assuming 100% purity) in DMSO (0.40 mL) was mixed with a solution of TCEP (5.8 μmol, 1.2 eq, 127 mM) in 20 mM HEPES pH 7.4 (45 μL). The reaction mixture was incubated on a Stuart rotator at room temperature and the reaction was monitored by RP-C₈-HPLC. The reaction mixture comprising DBCO-PEG₃₆-SH (Compound 59) was used without further purification in the next step.

### Step 3. Synthesis of DBCO-PEG₃₆-[S-MAL]-DUPA (Compound 61)

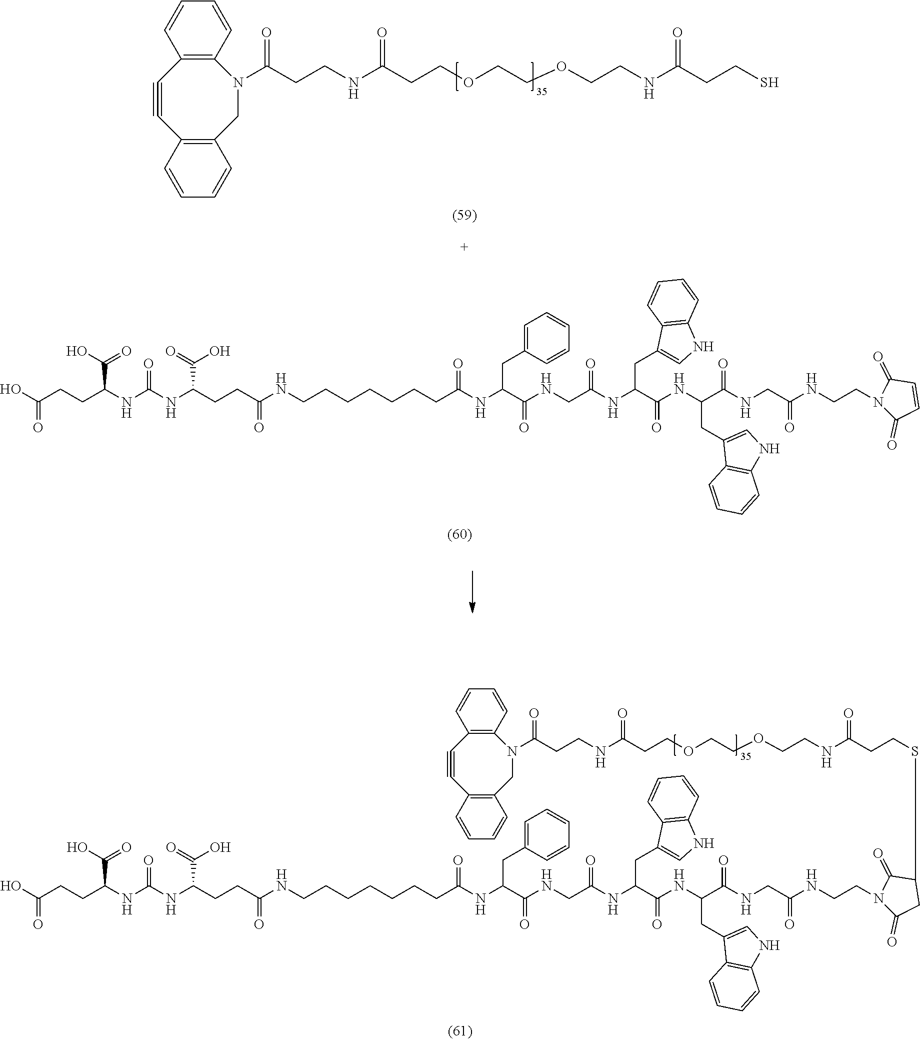

A solution of DUPA-MAL (Compound 60; 4.0 μmol, 1.0 eq, 2.5 mM) in 20 mM HEPES pH 7.4 (1.6 mL) was added to the solution of DBCO-PEG₃₆-SH (Compound 59; 364 μL, 4.0 μmol, 1.0 eq) prepared in Step 2 and the reaction mixture was incubated on a Stuart rotator at room temperature and monitored by RP-C₈-HPLC. After 15 min, an additional amount of DUPA-MAL (320 μL, 0.3 μmol, 0.1 eq) was added. After a total of 30 minutes, DBCO-PEG₃₆-[S-MAL]-DUPA (Compound 61) was isolated following preparative RP-Cis HPLC and lyophilization of pooled fractions. The lyophilizate was analyzed by RP-HPLC-ELSD and RP-HPLC-ESI⁺ qTOF mass spectrometry (DBCO-PEG₃₆-[S-MAL]-DUPA (7 mg) calculated monoisotopic mass: 3236.62 Da; measured: 3236.65 Da).

### Step 4. Synthesis of LPEI-1-[N₃:DBCO]-PEG₃₆-[S-MAL]-DUPA (Compounds 56a and 56b)

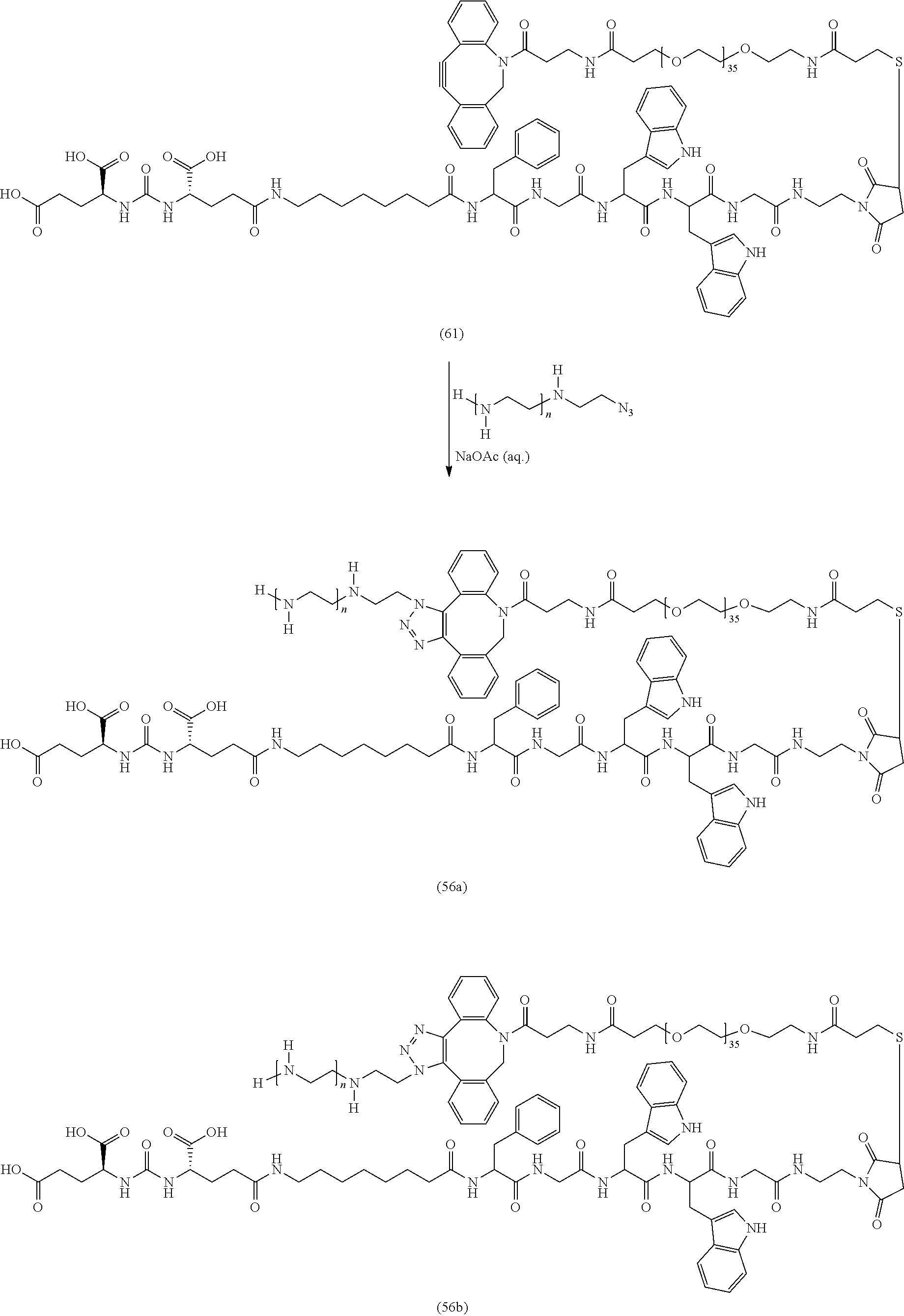

LPEI-N₃ solution (2.5 mL, 2.0 μmol, 1.0 eq) in 50 mM acetate buffer pH 4.0 was slowly added to 4.0 mL of a solution of DBCO-PEG₃₆-[S-MAL]-DUPA (Compound 61; 2.5 μmol, 1.2 eq, 1 mM) in 50 mM acetate buffer pH 4.0. The mixture was incubated at room temperature on a Stuart rotator and protected from light. After 20 hours, the reaction mixture was supplemented with acetonitrile (0.78 mL) and TFA (79 μL). LPEI-l-[N₃:DBCO]-PEG₃₆-[S-MAL]-DUPA was isolated as a mixture of regioisomers 56a and 56b using RP-C₁₈ preparative HPLC and characterized by analytical RP-C₈-HPLC, copper assay and spectrophotometry at 280 nm for determination of the DUPA content. The lyophilisate had a weight percentage in LPEI of 28% w/w and a LPEI to DUPA ratio of 1/1.08.

### Step 5. Preparation of LPEI-l-[N₃:DBCO]-PEG₃₆-[S-MAL]-DUPA (Compound 56a and 56b) HEPES Salt

LPEI-l-[N₃:DBCO]-PEG₃₆-[S-MAL]-DUPA (Compound 56a and 56b) TFA salt (24.9 mg, w_{LPEI}=28%, 7.0 mg in total LPEI) was dissolved in 0.8 mL 20 mM HEPES pH 7.2. Two centrifugal filters (Amicon Ultra—0.5 mL, 10 kDa MWCO) were filled with 400 μL of LPEI-l-[N₃:DBCO]-PEG₃₆-[S-MAL]-DUPA solution each, centrifugated one time at 14000 g for 30 minutes and then three times after addition of 400 μL 20 mM HEPES, pH 7.2. About 269 μL of LPEI-l-[N₃:DBCO]-PEG₃₆-[S-MAL]-DUPA (Compound 56a and 56b) HEPES salt solution were recovered and supplemented with 2.4 mL 20 mM HEPES, pH 7.2. The concentration of the solution was determined by copper assay to be 2.5 mg/mL in total LPEI.

### Example 16

### Synthesis of LPEI-l-[N₃:DBCO]-PEG₃₆-[MAL-S]-MTX (Compounds 62a and 62b)

Compounds 62a and 62b were synthesized as a mixture of regioisomers 62a and 62b according to the schemes below. Thiol-modified methotrexate MTX-SH (Compound 68) was prepared using solid phase synthesis. Compound 68 was condensed via Michael addition with DBCO-PEG₃₆-MAL (Compound 36), and the resulting DBCO-PEG₃₆-MTX was reacted with LPEI-N₃ to give Compounds 62a and 62b.

### Step 1. Synthesis of Fmoc-Glu-(OtBu)-cysteamine-4-methoxy trityl resin (Compound 64)

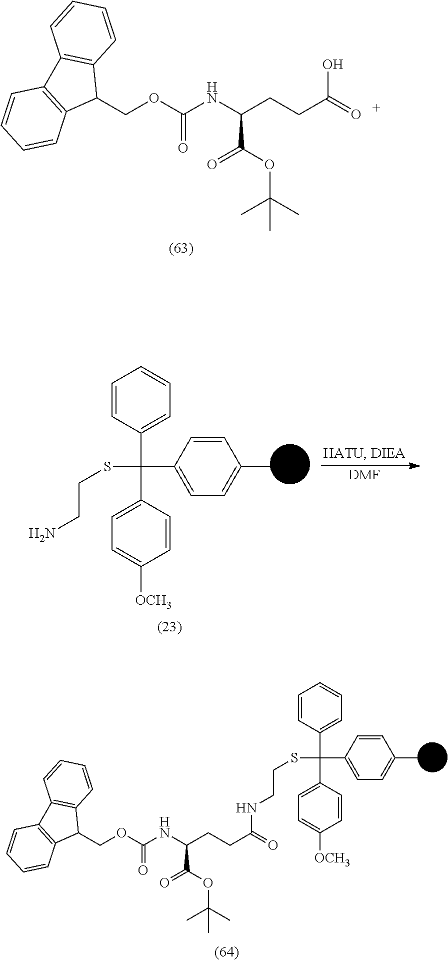

A solution of Fmoc-Glu-(OtBu) (Compound 63; 242 μmol, 5 eq, 242 mM) in DMF (1 mL) was added to a solution of HATU (246 μmol, 1 eq, 246 mM) in DMF (1 mL) and DIEA (42 μL, 250 μmol, 5 eq). After 3 min the reaction mixture was added to cysteamine 4-methoxytrityl resin (Compound 23; 51.1 mg, 50 μmol, 1.0 eq). The reaction mixture was incubated on a shaker at room temperature. After one hour, the reaction mixture was filtered and the Fmoc-Glu-(OtBu)-cysteamine-4-methoxy trityl resin (Compound 64) was washed with DMF (3×10 mL), DCM (3×10 mL) and MeOH (3×10 mL).

### Step 2. Synthesis of Glu-(OtBu)-cysteamine-4-methoxy trityl resin (Compound 65)

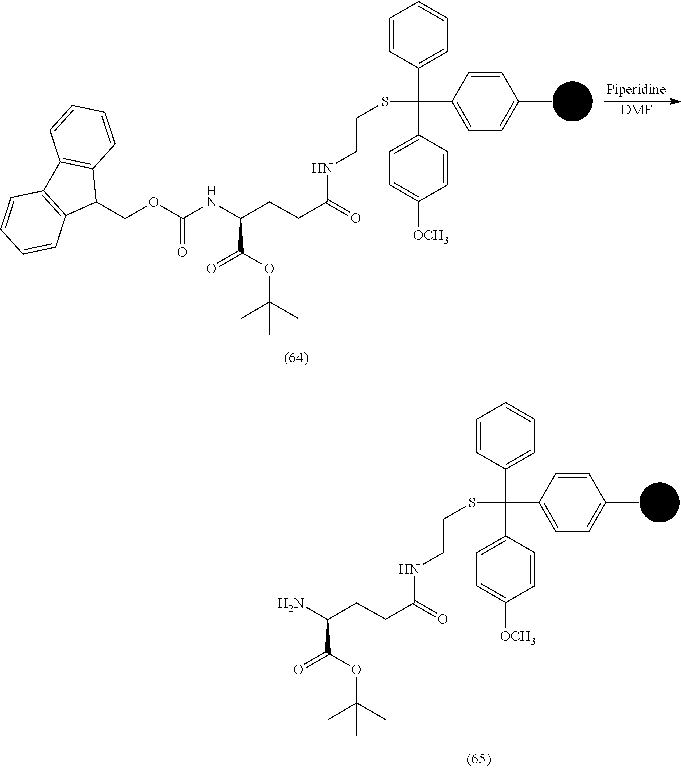

A solution of 25% piperidine in DMF (5 mL) was added to the Fmoc-Glu-(OtBu)-cysteamine-4-methoxy trityl resin (Compound 64) prepared in Step 1 and the reaction mixture was manually stirred for about 10 minutes. The resin was filtered and washed with DMF (3×10 mL), DCM (3×10 mL) and MeOH (3×10 mL) to give Glu-(OtBu)-cysteamine-4-methoxy trityl resin (Compound 65).

### Step 3. Synthesis of MTX-4-methoxy trityl resin (Compound 67)

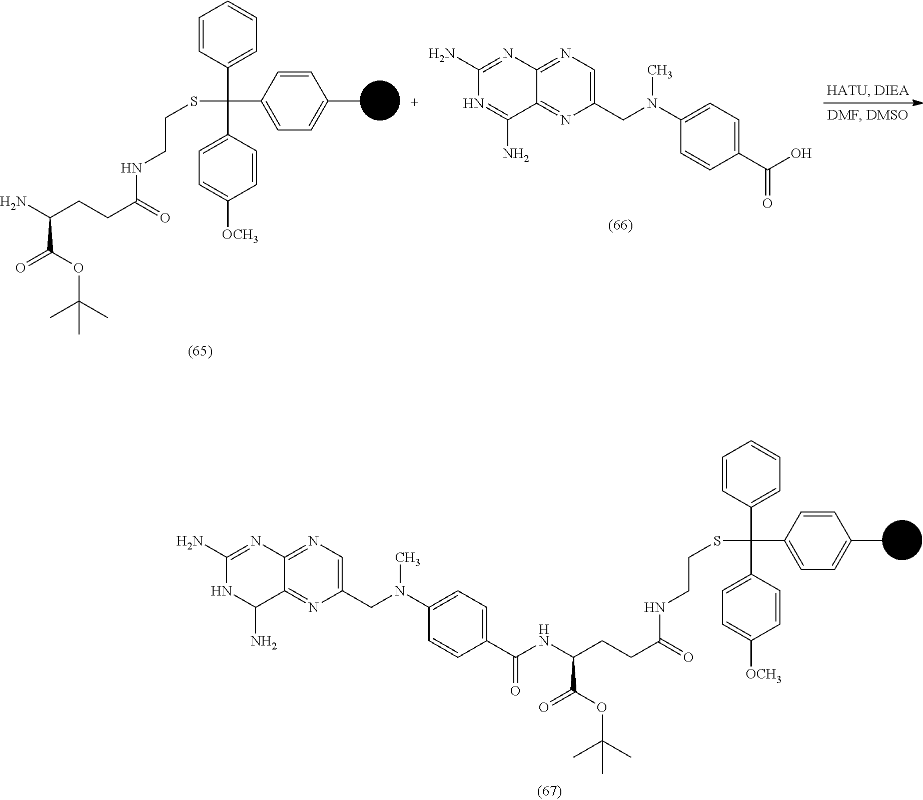 [SMILES: COc1ccc(C(SCCNC(=O)CC[C@H](N)C(=O)OC(C)(C)C)(c2ccccc2)c2ccc(C)cc2)cc1 ; CN(Cc1cnc2nc(N)nc(N)c2n1)c1ccc(C(=O)O)cc1 ; COc1ccc(C(SCCNC(=O)CC[C@H](NC(=O)c2ccc(N(C)Cc3cnc4c(n3)C(N)NC(N)=N4)cc2)C(=O)OC(C)(C)C)(c2ccccc2)c2ccc(C)cc2)cc1]

A solution of N¹⁰-Methyl-4-amino-4-deoxypteroic acid (MADOPA; Compound 66; 154 μmol, 3 eq, 17 mM) in DMF/DMSO (2:1) (9 mL) was mixed with a solution of HATU (146 μmol, 3 eq, 146 mM) in DMF (1 mL) and DIEA (25 μL, 147 μmol, 3 eq). The reaction mixture was mixed for 3 minutes and then added to 50 μmol (1 eq) of the Glu-(OtBu)-cysteamine-4-methoxy trityl resin (Compound 65) prepared in Step 2. The reaction mixture was transferred to a glass column with glass frit and was filtered and washed with DMSO (3×10 mL), DMF (3×10 mL), DCM (3×10 mL) and MeOH (3×10 mL) to give MTX-4-methoxy trityl resin (Compound 67).

### Step 4. Synthesis of MTX-SH (Compound 68)

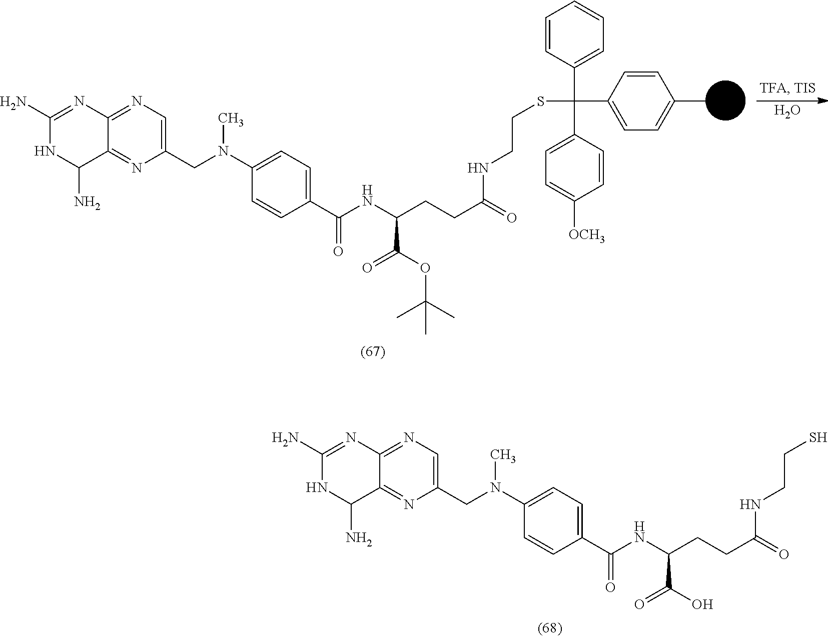 [SMILES: COc1ccc(C(SCCNC(=O)CC[C@H](NC(=O)c2ccc(N(C)Cc3cnc4c(n3)C(N)NC(N)=N4)cc2)C(=O)OC(C)(C)C)(c2ccccc2)c2ccc(C)cc2)cc1 ; O ; CN(Cc1cnc2c(n1)C(N)NC(N)=N2)c1ccc(C(=O)N[C@@H](CCC(=O)NCCS)C(=O)O)cc1]

A solution of TFA/TIS/H₂O (95:2.5:2.5) (4 mL) was added to the MTX-4-methoxy trityl resin (Compound 67) prepared in Step 3. The reaction mixture was incubated for one hour on a shaker at room temperature. The resin was filtered, and the filtrate was recovered and concentrated under nitrogen flow for 15 minutes to evaporate TFA. Cold diethyl ether (10 mL) was added. The resultant precipitate was washed with cold diethyl ether (4.5 mL). A brown-yellowish solid material comprising MTX-SH (Compound 68) was recovered and analyzed by HPLC-ESI⁺ single quadrupole mass spectrometry (calculated masses [M+1]⁺: 514.20 Da, [M+2]⁺: 257.80 Da; measured masses [M+1]⁺: 515.0 Da, [M+2]⁺: 258.00 Da).

### Step 5. Synthesis of DBCO-PEG₃₆-MTX (Compound 69)

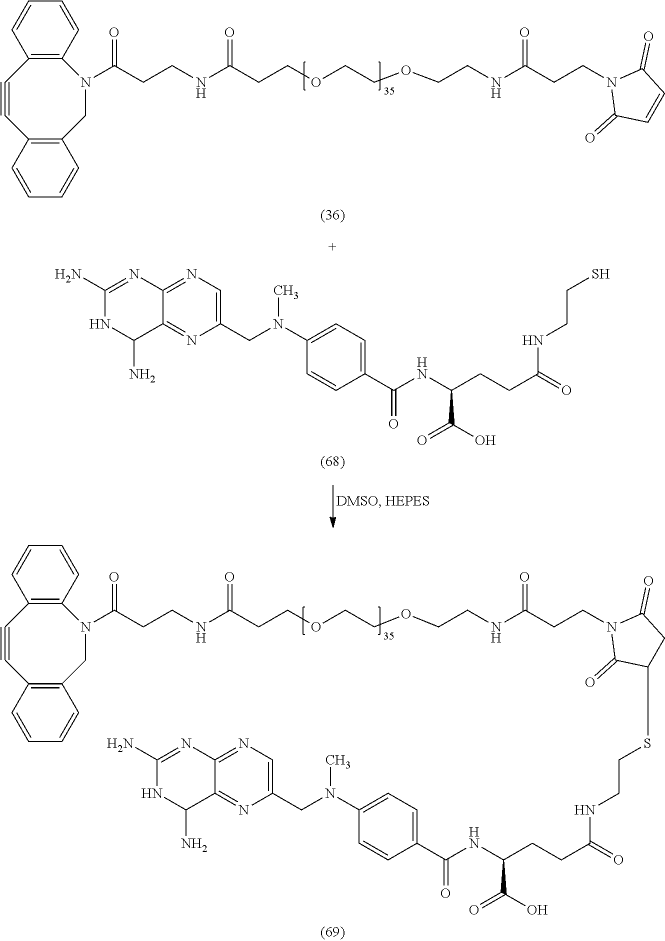

A solution of MTX-SH (Compound 68; 8 mol, 0.9 eq, 1.1 mM in thiol) in DMSO/20 mM HEPES pH 7.4 (1:9) (7.0 mL) was mixed with a solution of DBCO-PEG₃₆-MAL (Compound 36; 9 mol, 1.0 eq, 41 mM) in DMSO (220 μL). The reaction mixture was incubated on a Stuart rotator at room temperature, protected from light and was monitored by RP-C₈-HPLC. After 1.5 hr acetonitrile (0.8 mL) was added to the reaction mixture. DBCO-PEG₃₆-MTX (Compound 69; 14 mg) was isolated following RP-C₁₈ preparative HPLC and lyophilization of pooled fractions and analyzed by HPLC-ESI⁺ qTOF mass spectrometry (calculated monoisotopic mass: 2596.32 Da; measured: 2596.35 Da).

### Step 6. Synthesis of LPEI-l-[N₃:DBCO]-PEG₃₆-[MAL-S]-MTX (Compounds 62a and 62b)

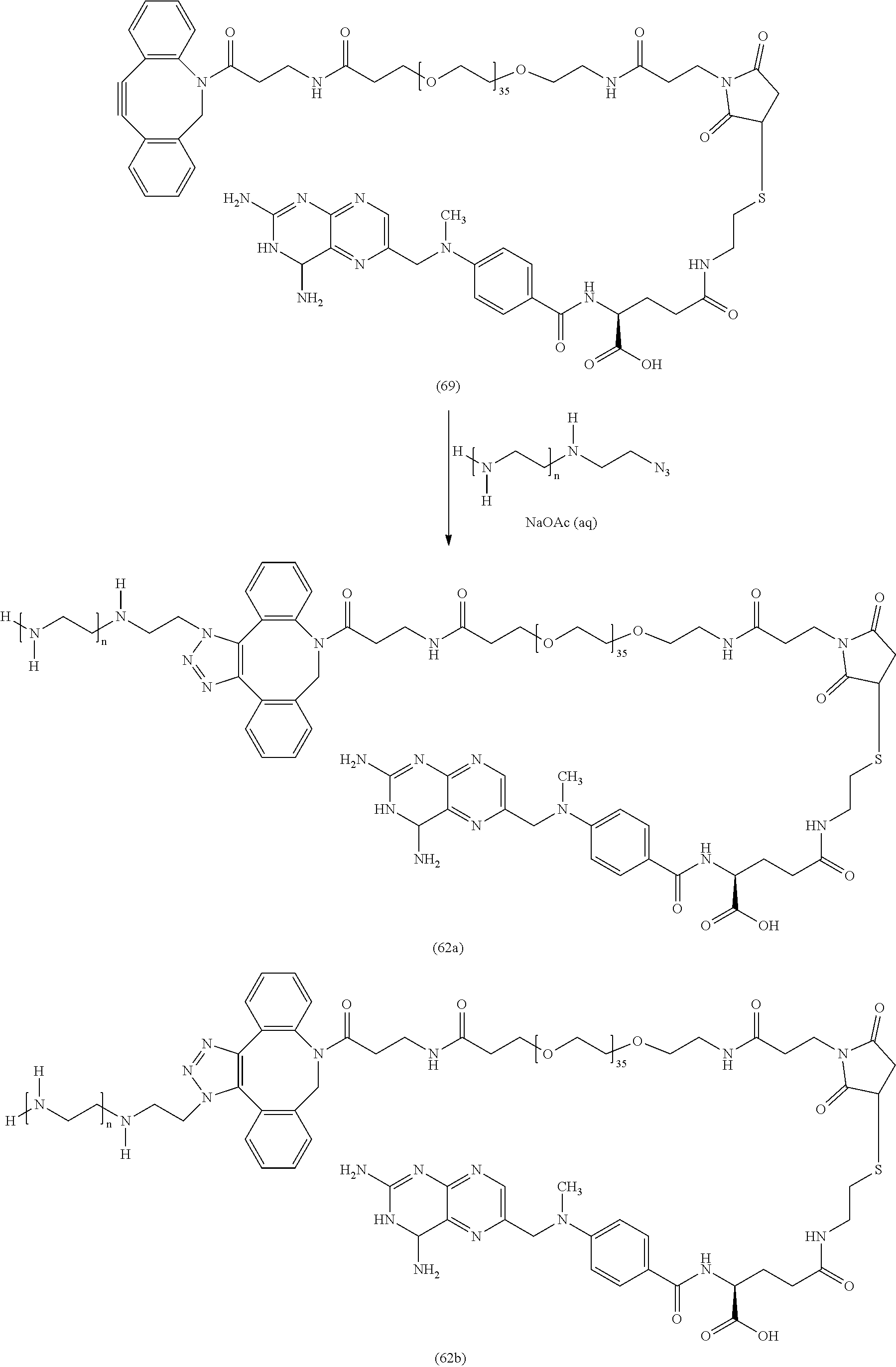

LPEI-N₃ solution (4.2 mL, 5.0 mol, 0.9 eq, 1.2 mM) in 50 mM acetate buffer pH 4.0 was slowly added to 5.0 mL of a solution of DBCO-PEG₃₆-MTX (Compound 69; 5.4 mol, 1.0 eq, 1.1 mM) in 50 mM acetate buffer pH 4.0. The reaction mixture was incubated at room temperature on a Stuart rotator, protected from light, and monitored by RP-C₈-HPLC. After twenty hours of reaction, the mixture was supplemented with acetonitrile (1.0 mL) and with TFA (100 μL). LPEI-l-[N₃:DBCO]-PEG₃₆-[MAL-S]-MTX was isolated as a mixture of regioisomers 62a and 62b using RP-C₁₈ preparative HPLC. Pooled fractions were lyophilized to give 90 mg of a fluffy white-yellow solid which was characterized by RP-C₈-HPLC, copper assay and spectrophotometry at 305 nm for determination of the methotrexate content. The lyophilisate had a weight percentage in LPEI of 34% w/w and a LPEI to methotrexate ratio of 1/1.0.

### Step 7. Preparation of LPEI-l-[N₃:DBCO]-PEG₃₆-[MAL-S]-MTX (Compounds 62a and 62b) HEPES Salt

LPEI-l-[N₃:DBCO]-PEG₃₆-[MAL-S]-MTX (Compounds 62a and 62b) TFA salt (23.8 mg, w_{LPEI}=34%, 8.1 mg in total LPEI) were dissolved in 0.8 mL 20 mM HEPES pH 7.2. Two centrifugal filters (Amicon Ultra—0.5 mL, 10 kDa MWCO) were filled with 400 μL of LPEI-l-[N₃:DBCO]-PEG₃₆-[MAL-S]-MTX solution each, centrifuged one time at 14′000 g for 30 minutes and then three times after addition of 400 μL 20 mM HEPES, pH 7.2. About 250 μL of LPEI-l-[N₃:DBCO]-PEG₃₆-[MAL-S]-MTX-HEPES salt solution were recovered and supplemented with 2.3 mL 20 mM HEPES, pH 7.2. The concentration of the solution was determined by copper assay to be 2.6 mg/mL in total LPEI.

### Example 17

### Synthesis of LPEI-l-[N₃:DBCO]-PEG₃₆-hEGF (Compounds 70a and 70b)

LPEI-l-[N₃:DBCO]-PEG₃₆-hEGF was prepared as a mixture of regioisomers 70a and 70b according to the schemes below. DBCO-PEG₃₆-TFP (Compound 52) was condensed with hEGF, and the resulting DBCO-PEG₃₆-hEGF (Compound 71) was reacted with LPEI-N₃ to give Compounds 70a and 70b.

### Step 1. Synthesis of DBCO-PEG₃₆-hEGF (Compound 71)

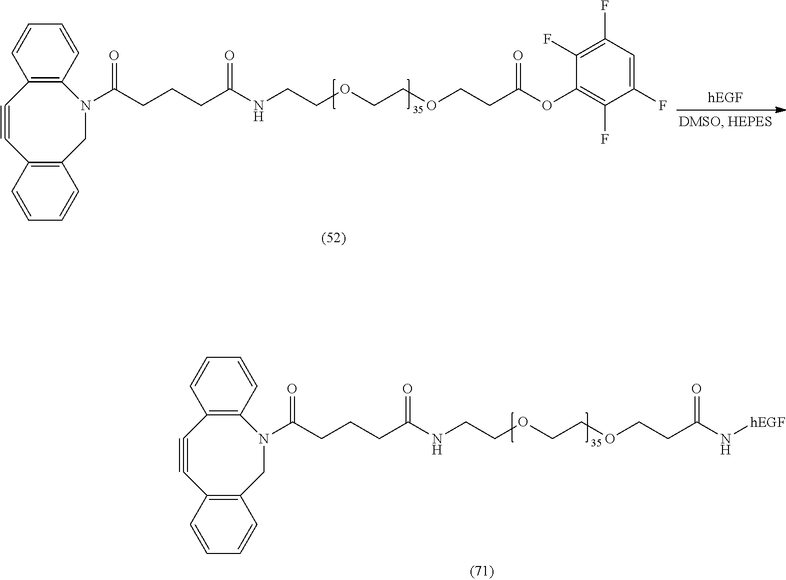

A solution of DBCO-PEG₃₆-TFP (Compound 52; 128 μmol, 1.4 eq, 64 mM) in DMSO (2.0 mL) was slowly added to a solution of hEGF (92 μmol, 1.0 eq, 2.6 mM) in 20 mM HEPES pH 7.5 (35 mL). The reaction mixture was stirred in a round-bottom flask and the reaction was monitored by RP-C₈-HPLC. After one hour, an additional amount of DBCO-PEG₃₆-TFP (140 μL, 9 μmol, 0.1 eq, 64 mM) was added. After a total of 1.5 hrs, acetonitrile (4 mL) was added to the reaction mixture and the pH adjusted to 3.5. DBCO-PEG₃₆-hEGF (Compound 71) was isolated following RP-C₁₈ preparative HPLC. Pooled fractions were lyophilized to give 310 mg of a fluffy white solid which was analyzed by RP-HPLC-ELSD and RP-HPLC-ESI⁺ qTOF mass spectrometry (DBCO-PEG₃₆-hEGF calculated monoisotopic mass: 8168.79 Da; measured: 8168.80 Da).

### Step 2. Synthesis of LPEI-l-[N₃:DBCO]-PEG₃₆-hEGF (Compounds 70a and 70b)

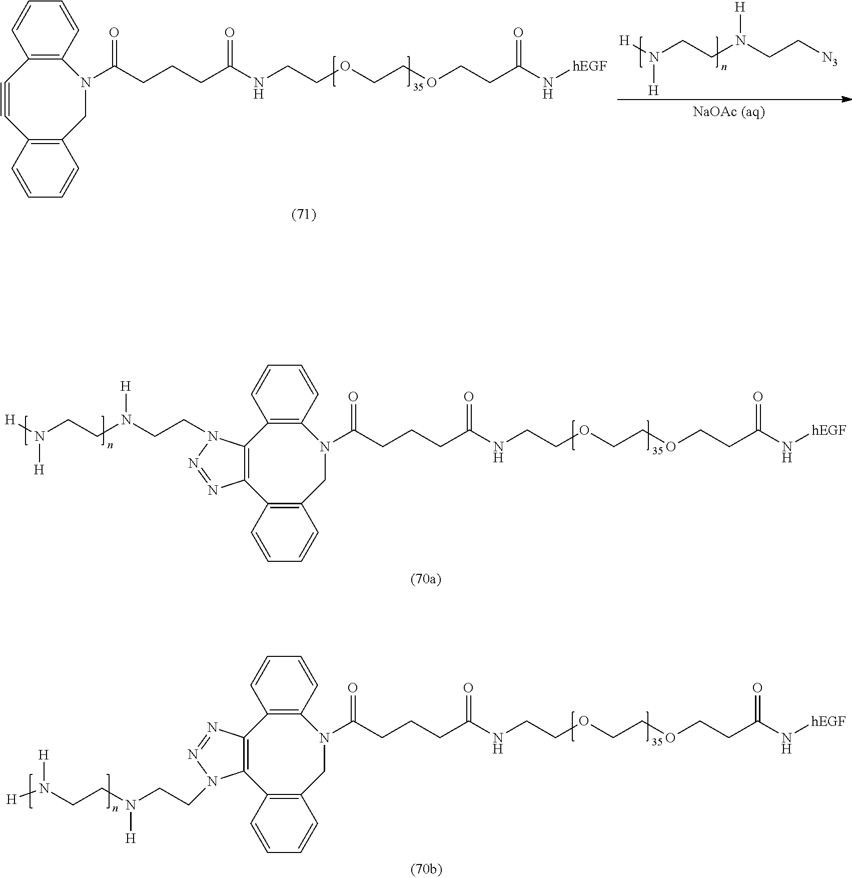

LPEI-N₃ solution (24 mL, 23 μmol, 1.0 eq, 0.94 mM) in 50 mM acetate buffer pH 4.0 was slowly added to a solution of DBCO-PEG₃₆-hEGF (Compound 71; 16 mL, 22 μmol, 1.0 eq) in 50 mM acetate buffer pH 4.0. The reaction mixture was stirred in a round-bottom flask and monitored by RP-C₈-HPLC. After a total of 72 hours, acetonitrile (4 mL) and TFA (400 μL) were added to the reaction mixture. LPEI-l-[N₃:DBCO]-PEG₃₆-hEGF was isolated as a mixture of regioisomers 70a and 70 b using RP-C₁₈ preparative HPLC. Pooled fractions were lyophilized (505 mg) and characterized by RP-C₈-HPLC, copper assay and spectrophotometry at 280 nm for determination of the hEGF content.

The lyophilizate was dissolved in 50 mM acetate, pH 4.5 and processed by TFF (10 kDa MWCO membrane) to remove TFA residues. A solution of LPEI-l-[N₃:DBCO]-PEG₃₆-hEGF (Compounds 70a and 70b) acetate (42 mL) was recovered and characterized by RP-C₈-HPLC, copper assay and spectrophotometry at 280 nm for determination of the hEGF content. The solution had a concentration of 2.6 mg/mL in total LPEI and a LPEI to hEGF ratio of 1/1.0.

### Example 18

### Synthesis of LPEI-l-[N₃:DBCO]-PEG₃₆-[S-MAL]-hEGF (Compounds 72a and 72b)

LPEI-l-[N₃:DBCO]-PEG₃₆-[S-MAL]-hEGF was prepared as a mixture of regioisomers 72a and 72b according to the schemes below. DBCO-PEG₃₆-[S-MAL]-hEGF (Compound 74) was prepared by condensing hEGF with MCC-hEGF (Compound 73). The resulting DBCO-PEG₃₆-[S-MAL]-hEGF (Compound 74) was reacted with LPEI-N₃ to give Compounds 72a and 72b.

### Step 1. Synthesis of DBCO-PEG₃₆-[S-MAL]-hEGF (Compound 74)

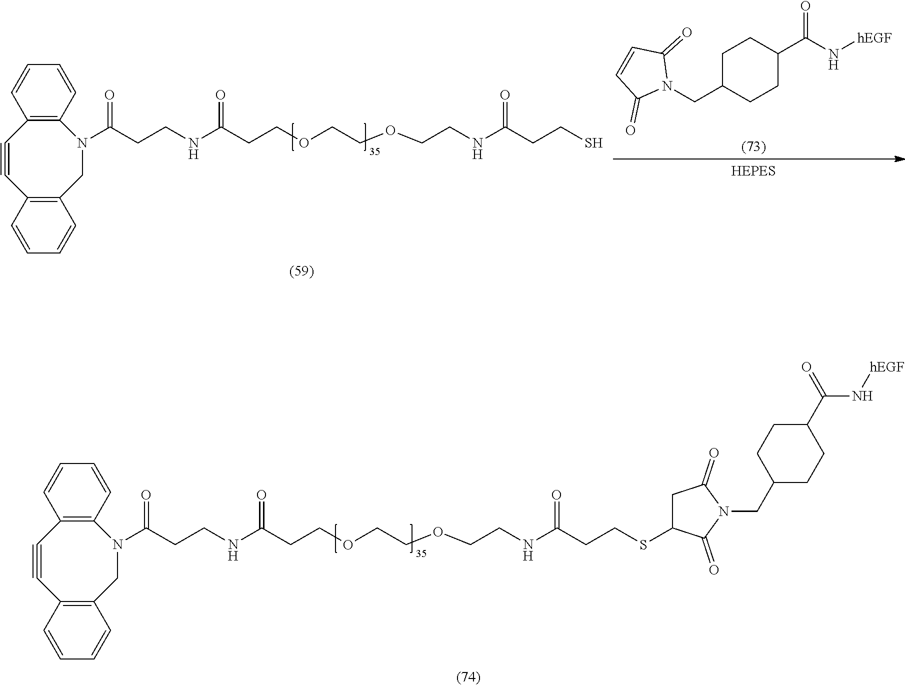

A solution of DBCO-PEG₃₆-SH (Compound 59) (230 μL, 3.0 μmol, 1.0 eq) was prepared as described in Example 15. A solution of MCC-hEGF (Compound 73; 2.9 μmol, 1.0 eq, 0.58 mM based on 77% measured peptide content; CBL Patras S.A. (Greece)) in 20 mM HEPES pH 7.2 (5.0 mL) was added and the reaction mixture was incubated on a Stuart rotator at room 10 temperature and was monitored by RP-C₈-HPLC. After 15 min, an additional amount of DBCO-PEG₃₆-SH solution (20 μL, 0.3 μmol, 0.1 eq) was added. After a total of 30 minutes, acetonitrile (0.56 mL) was added and the reaction mixture was purified by RP-C₁₈ preparative HPLC. DBCO-PEG₃₆-[S-MAL]-hEGF (Compound 74) was isolated and pooled fractions containing Compound 74 were lyophilized. The lyophilisate (15 mg) was analyzed by RP-HPLC-ELSD and RP-HPLC-ESI⁺ qTOF mass spectrometry (DBCO-PEG₃₆-[S-MAL]-hEGF calculated monoisotopic mass: 8450.90 Da; measured: 8450.97 Da).

### Step 2. Synthesis of LPEI-1-[N₃:DBCO]-PEG₃₆-[S-MAL]-hEGF (Compounds 72a and 72b)

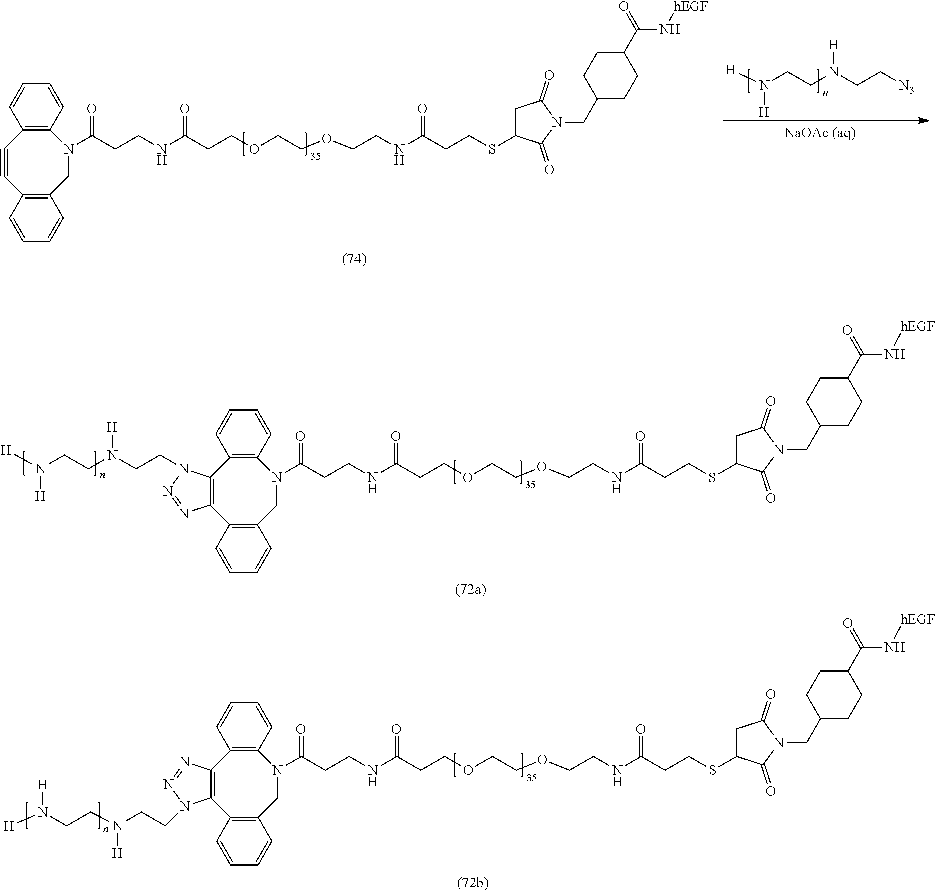

LPEI-N₃ solution (2.5 mL, 2.0 μmol, 1.2 eq, 0.84 mM) in 50 mM acetate buffer pH 4.0 was slowly added to 4.0 mL of a solution of DBCO-PEG₃₆-[S-MAL]-hEGF (Compound 74; 1.7 μmol, 1.0 eq, 0.43 mM) in 50 mM acetate buffer pH 4.0. The mixture was incubated at room temperature on a Stuart rotator and protected from light. After 20 hours, the reaction mixture was supplemented with acetonitrile (0.72 mL) and TFA (73 μL). LPEI-l-[N₃:DBCO]-PEG₃₆-[S-MAL]-hEGF was isolated as a mixture of regioisomers 72a and 72b using RP-Cis preparative HPLC and characterized by RP-C₈-HPLC, copper assay and spectrophotometry at 280 nm for determination of the hEGF content. The lyophilisate had a weight percentage in LPEI of 25% w/w and a LPEI to hEGF ratio of 1/1.09.

### Step 3. Preparation of LPEI-l-[N₃:DBCO]-PEG₃₆-[S-MAL]-hEGF (Compounds 72a and 72b) HEPES Salt

LPEI-l-[N₃:DBCO]-PEG₃₆-[S-MAL]-hEGF (Compounds 72a and 72b) TFA salt (26.4 mg, w_{LPEI}=25%, 6.6 mg in total LPEI) was dissolved in 0.8 mL 20 mM HEPES pH 7.2. Two centrifugal filters (Amicon Ultra—0.5 mL, 10 kDa MWCO) were filled with 400 μL of LPEI-l-[N₃:DBCO]-PEG₃₆-[S-MAL]-hEGF solution each, centrifugated one time at 14000 g for 30 minutes and then three times after addition of 400 μL 20 mM HEPES, pH 7.2. About 212 μL of LPEI-l-[N₃:DBCO]-PEG₃₆-[S-MAL]-hEGF (Compounds 72a and 72b) HEPES salt solution were recovered and supplemented with 2.3 mL 20 mM HEPES, pH 7.2. The concentration of the solution was determined by copper assay to be 2.3 mg/mL in total LPEI.

### Example 19

### Synthesis of LPEI-l-[N₃:DBCO]-PEG₃₆-[MAL-S]-CysGE11 (Compounds 75a and 75b)

LPEI-l-[N₃:DBCO]-PEG₃₆-[MAL-S]-GE11 was synthesized as a mixture of regioisomers 75a and 75b in two steps according to the schemes below. In the first step, human peptide Cys-GE11 (Compound 76) was coupled to (DBCO-PEG₃₆-MAL; Compound 36) in 20 mM HEPES buffer to produce DBCO-PEG₃₆-[MAL-S]-CysGE11 (Compound 77). In the second step, DBCO-PEG₃₆-[MAL-S]-CysGE11 was conjugated to LPEI-N₃ to produce LPEI-l-[N₃:DBCO]-PEG₃₆-[MAL-S]-CysGE11 (Compounds (75a and 75b).

### Step 1. Synthesis of DBCO-PEG₃₆-[MAL-S]-CysGE11

A solution of CysGE11 peptide (Compound 76; 6.5 μmol, 1.0 eq, 0.93 mM) in 20 mM HEPES pH 7.4 (7.0 mL) was mixed with a solution of TCEP (6.5 μmol, 1.0 eq, 85 mM) in 20 mM HEPES pH 7.4 (76 μL). A solution of DBCO-PEG₃₆-MAL (Compound 36; 7.8 μmol, 1.2 eq, 24 mM) in DMSO (0.32 mL) was then added and the reaction mixture was incubated on a Stuart rotator at room temperature. After a total of 30 minutes, acetonitrile (0.8 mL) was added to the reaction mixture which was purified by RP-C₁₈ preparative HPLC. Lyophilization of pooled fractions yielded DBCO-PEG₃₆-[MAL-S]-CysGE11 (Compound 77) as a solid (13 mg; calculated monoisotopic mass: 3725.85 Da; measured: 3725.90 Da).

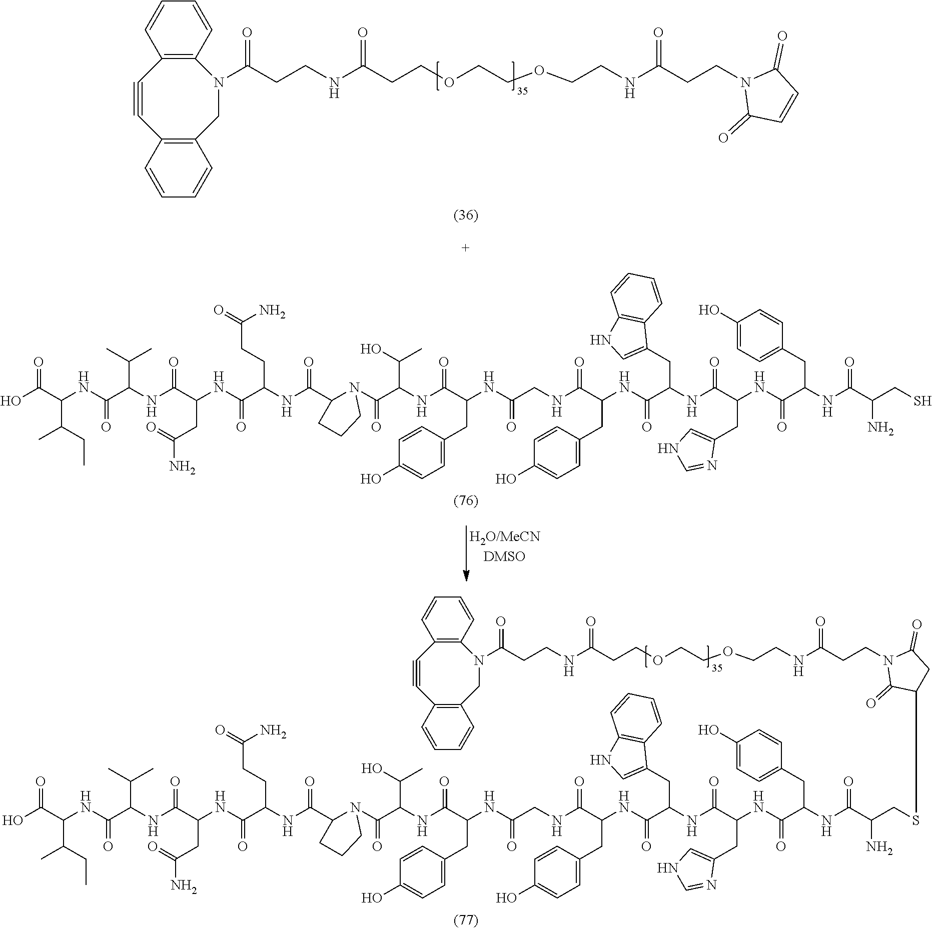

### Step 2. Synthesis of LPEI-l-[N₃:DBCO]-PEG₃₆-[MAL-S]-CysGE11

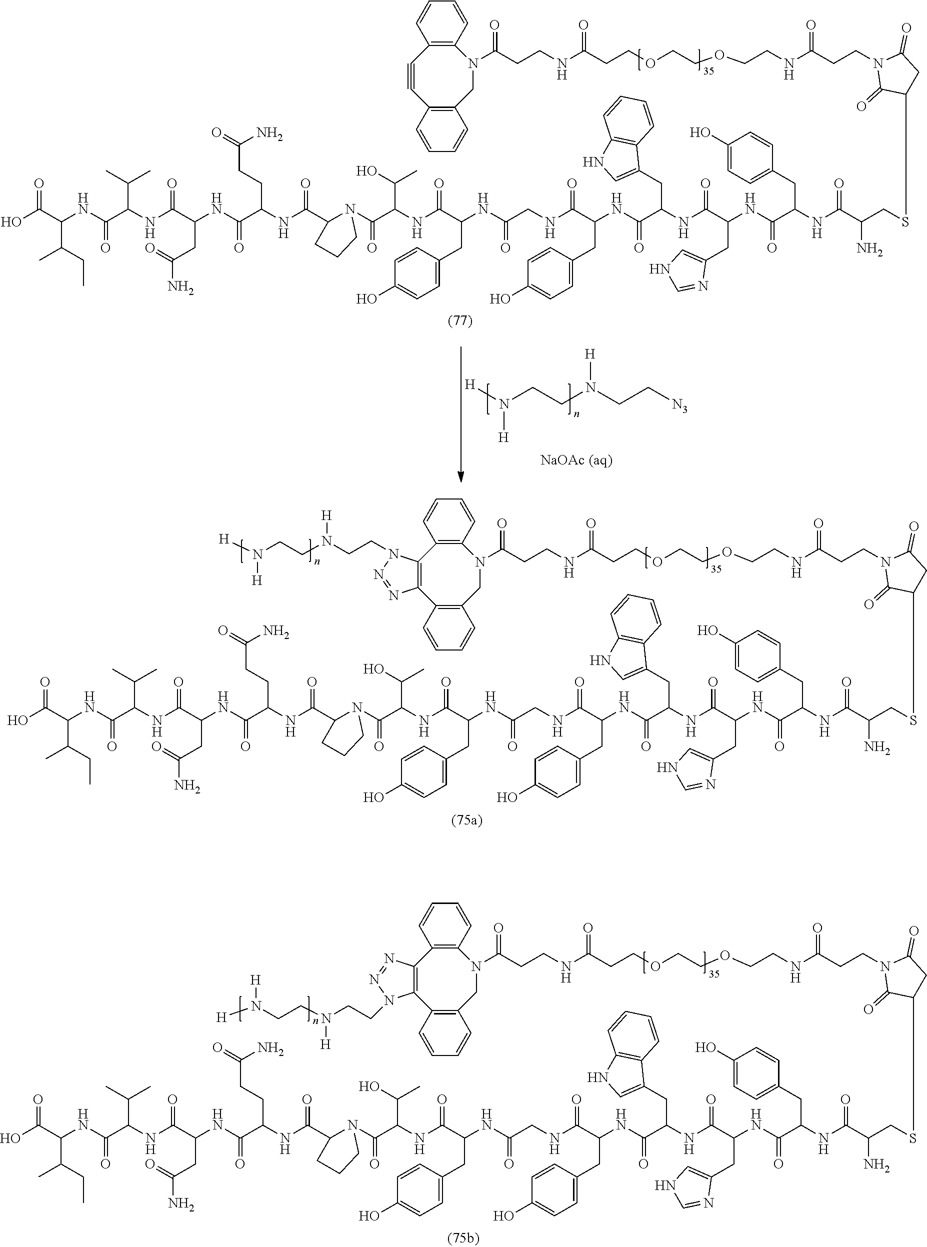

LPEI-N₃ solution (3.0 mL, 2.5 mol, 1.0 eq, 0.84 mM) in 50 mM acetate buffer pH 4.0 was slowly added to 4.0 mL of a solution of DBCO-PEG₃₆-[MAL-S]-CysGE11 (Compound 77; 4.3 μmol, 1.7 eq, 1.1 mM) in 50 mM acetate buffer pH 4.0. The mixture was incubated at room temperature on a Stuart rotator and protected from light. After 16 hours, acetonitrile (0.78 mL) and TFA (78 μL) were added to the reaction mixture which was purified using RP-Cis preparative HPLC. Pooled fractions were lyophilized to yield LPEI-l-[N₃:DBCO]-PEG₃₆-[MAL-S]-CysGE11 (60 mg) as a mixture of regioisomers 75a and 75b, which were characterized by RP-C₈-HPLC, copper assay and spectrophotometry at 280 nm to determination the peptide content. The lyophilizate had a weight percentage in LPEI of 27% w/w and a LPEI to CysGE11 ratio of 1/1.1.

### Step 3. Preparation of LPEI-l-[N₃:DBCO]-PEG₃₆-[MAL-S]-CysGE11 (Compounds 75a and 75b) HEPES Salt

LPEI-l-[N₃:DBCO]-PEG₃₆-[MAL-S]-CysGE11 (Compounds 75a and 75b) TFA salt (27.3 mg, w_{LPEI}=27%, 7.4 mg in total LPEI) was dissolved in 0.8 mL 20 mM HEPES pH 7.2. Two centrifugal filters (Amicon Ultra—0.5 mL, 10 kDa MWCO) were filled with 400 μL each of LPEI-l-[N₃:DBCO]-PEG₃₆-[MAL-S]-CysGE11 solution each, centrifugated one time at 14000 g for 30 minutes and then three times after addition of 400 μL 20 mM HEPES, pH 7.2. About 245 μL of LPEI-l-[N₃:DBCO]-PEG₃₆-[MAL-S]-CysGE11-HEPES salt solution were recovered and supplemented with 2.3 mL 20 mM HEPES, pH 7.2. The concentration of the solution was determined by copper assay (2.6 mg/mL in total LPEI and a LPEI to CysGE11 ratio of 1/1.1).

### Example 20

### Synthesis of LPEI-l-[N₃:DBCO]-PEG₃₆-(GalNAc)₃ (Compounds 78a and 78b)

### Step 1. Synthesis of DBCO-PEG₃₆-(GalNAc)₃ (Compound 80)

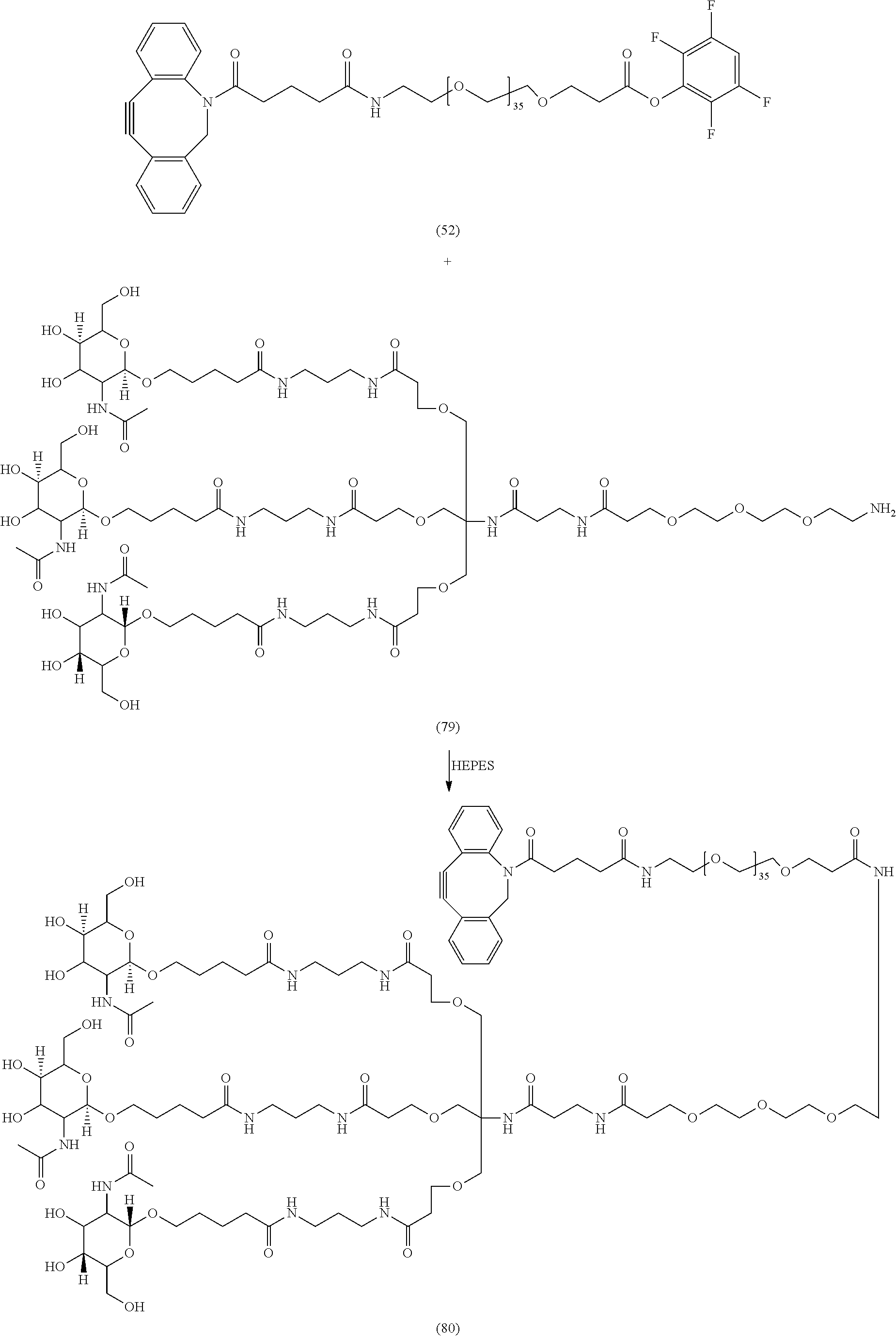

A solution of (GalNAc)₃—PEG₃-NH₂ (Compound 79; 5.6 μmol, 1.0 eq, 7.2 mM) in 20 mM HEPES pH 7.4 (0.5 mL) was added to a solution of DBCO-PEG₃₆-TFP (Compound 52; 7.5 μmol, 1.3 eq, 48 mM) in DMSO (0.155 mL). The reaction mixture was placed on a Stuart rotator at room temperature and the reaction was monitored by RP-C₈-HPLC. After 2 hours an additional 3.0 mL of 20 mM HEPES buffer pH 7.4 was added. After a total of 20 hours, milliQ water (3.4 mL) and acetonitrile (0.78 mL) were added to the reaction mixture, which was purified using RP-C₁₈ preparative HPLC. Pooled fractions containing purified DBCO-PEG₃₆-(GalNAc)₃ (Compound 80) were lyophilized to yield a solid (10 mg; ESI⁺ qTOF mass spectrometry, calculated monoisotopic: mass: 3646.00 Da; measured: 3646.02 Da).

### Step 2. Synthesis of LPEI-l-[N₃:DBCO]-PEG₃₆-GalNAc)₃ (Compounds 78a and 78b)

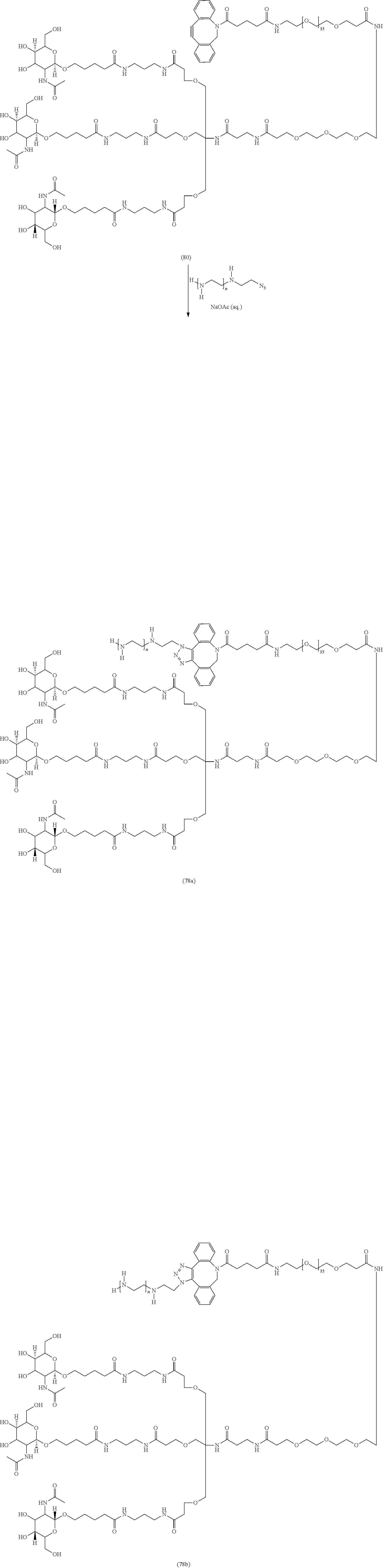

LPEI-N₃ solution (3.0 mL, 2.5 μmol, 1.0 eq, 0.84 mM) in 50 mM acetate buffer pH 4.0 was slowly added to 4.0 mL of a solution of DBCO-PEG₃₆-(GalNAc)₃ (Compound 80; 3.0 μmol, 1.2 eq, 0.76 mM) in 50 mM acetate buffer pH 4.0. The mixture was placed on a Stuart rotator and protected from light. After 16 hours, acetonitrile (0.78 mL) and TFA (79 μL) were added to the reaction mixture for preparative chromatography. LPEI-l-[N₃:DBCO]-PEG₃₆-(GalNAc)₃ (Compounds 78a and 78b) was isolated as a mixture of regioisomers 78a and 78b using RP-C₁₈ preparative HPLC and characterized by RP-C₈-HPLC and copper assay. Lyophilisate (63 mg) had a weight percentage in LPEI of 27% w/w.

### Step 3. Preparation of LPEI-l-[N₃:DBCO]-PEG₃₆-GalNAc)₃—HEPES Salt

LPEI-l-[N₃:DBCO]-PEG₃₆-(GalNac)₃ (Compounds 78a and 78b) TFA salt (42 mg, w_{LPEI}=27%, 11.3 mg in total LPEI) was solubilized in 1.6 mL 20 mM HEPES pH 7.2. Four centrifugal filters (Amicon Ultra—0.5 mL, 10 kDa MWCO) were filled with 400 μL of LPEI-l-[N₃:DBCO]-PEG₃₆-(GalNac)₃ solution each, centrifugated one time at 14000 g for 30 minutes and then three times after addition of 400 μL 20 mM HEPES, pH 7.2. About 418 μL of LPEI-l-[N₃:DBCO]-PEG₃₆-(GalNac)₃-HEPES salt solution were recovered and supplemented with 4.0 mL 20 mM HEPES, pH 7.2. The concentration of the solution (4.4 mL) was determined by copper assay (2.2 mg/mL in total LPEI).

### Example 21

### Synthesis of Me-LPEI-1-[N₃:BCN]-PEG₃₆-DUPA (Compound 81)

Me-LPEI-l-[N₃:BCN]-PEG₃₆-DUPA (Compound 81) was synthesized according to the schemes below. In a first step, HOOC-PEG₃₆-NH₂ (Compound 32) was coupled to N-succinimidyl 3-maleimidopropionate (Compound 33) by amide formation to produce HOOC-PEG₃₆-MAL (Compound 34). Endo-BCN-PEG₃₆-MAL (Compound 45) was prepared by condensing HOOC-PEG₃₆-MAL (Compound 34) with endo-BCN-PEG₂-NH₂ (Compound 44). In a next step, Compound 45 was condensed with Compound 12, and the resulting endo-BCN-PEG₃₆-[MAL-S]-DUPA (Compound 46) was reacted with Me-LPEI-N₃ to give Compound 81.

### Step 1: Synthesis of HOOC-PEG₃₆-MAL (Compound 34)

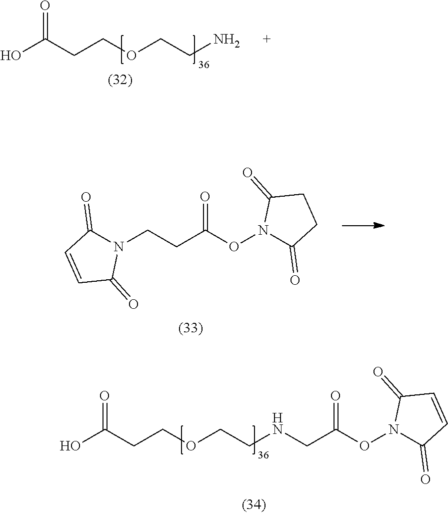

A solution of HOOC-PEG₃₆-NH₂ (Compound 32, 94 μmol, 1.0 eq, 234 mM) in DCM (0.40 mL) was mixed with a solution of N-succinimidyl 3-maleimidopropionate (Compound 33, 85 μmol, 0.9 eq, 184 mM) in DCM (0.83 mL). The reaction mixture was shaken on a Stuart rotator at room temperature and the reaction was monitored by RP-C₈ HPLC. At one hour into reaction, an additional amount of N-succinimidyl 3-maleimidopropionate (137 μL, 14 μmol, 0.15 eq) was added and at 1 h 15 min into reaction, an additional amount of N-succinimidyl 3-maleimidopropionate (83 μL, 9 μmol, 0.1 eq) was added. After a total of 1.5 hours, 4.5 mL of cold diethyl ether were added to induce precipitation followed by centrifugation. The precipitate was washed with 4.5 mL cold diethyl ether and 183 mg of HOOC-PEG₃₆-MAL (Compound 34) were recovered (calculated monoisotopic mass: 1825.03 Da; measured: 1825.02 Da).

### Step 2. Synthesis of endo-BCN-PEG₃₆-MAL (Compound 45)

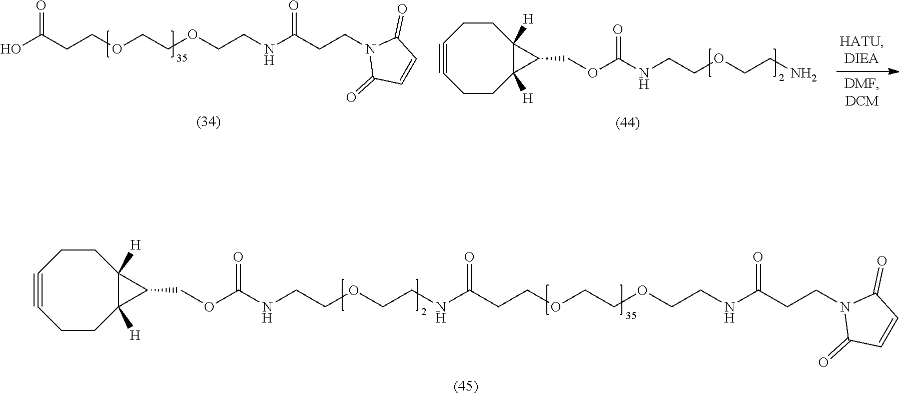

A solution of HOOC-PEG₃₆-MAL (Compound 34, 40 μmol, 1.0 eq, 99 mM) in DCM (0.40 mL) was mixed with a solution of HATU (36 μmol, 0.9 eq, 325 mM) in DMF (111 μL). The mixture was stirred for one minute and DIEA (14 μL, 80 μmol, 2.0 eq) was added. The mixture was stirred for three minutes and was mixed with a solution of endo-BCN-PEG₂-NH₂ (Compound 44, 32 μmol, 0.8 eq, 370 mM) in DCM (86 μL). The reaction mixture was shaken on a Stuart rotator at room temperature and the reaction was monitored by RP-C₈-HPLC. At 15 minutes into reaction, an additional amount of endo-BCN-PEG₂-NH₂ (54 μL, 20 μmol, 0.5 eq) was added and at 30 minutes into reaction, a further amount of endo-BCN-PEG₂-NH2 (32 μL, 12 μmol, 0.3 eq) was added. After a total of 45 minutes, 4.5 mL of n-hexane were added to induce precipitation followed by centrifugation. The precipitate was washed with 4.5 mL cold diethyl ether and 103 mg of endo-BCN-PEG₃₆-MAL (Compound 45) solid material were recovered (calculated monoisotopic mass: 2131.21 Da; measured: 2131.22 Da.

### Step 3. Synthesis of endo-BCN-PEG₃₆-[MAL-S]-DUPA (Compound 46)

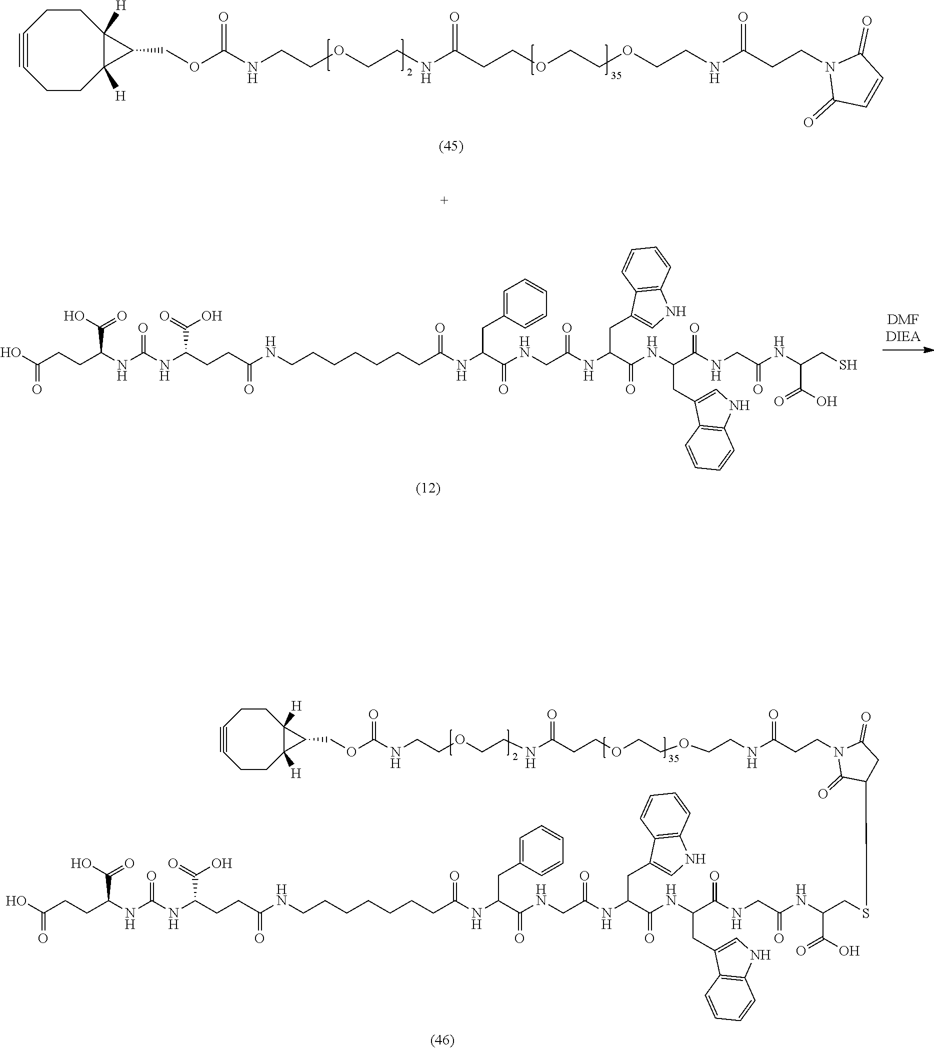

A solution of endo-BCN-PEG₃₆-MAL (Compound 45, 19 μmol, 1.0 eq, 93 mM) in DMF (0.20 mL) was mixed with a solution of DUPA-Aoc-Phe-Gly-Trp-Trp-Gly-Cys (Compound 12, 13 μmol, 0.7 eq, 77 mM) in DMF (173 μL) and DIEA (6 μL, 38 μmol, 2.0 eq). The reaction mixture was shaken on a Stuart rotator at room temperature and the reaction was monitored by RP-C₈ HPLC. At 1 hour into reaction, an additional amount of DUPA-Aoc-Phe-Gly-Trp-Trp-Gly-Cys (26 μL, 2 μmol, 0.1 eq) was added. After a total of 2 hours the mixture was purified using RP-C₁₈ preparative chromatography and pooled fractions containing endo-BCN-PEG₃₆-[MAL-S]-DUPA (Compound 46) were lyophilized to give 12 mg lyophilized product. The lyophilisate was analyzed by RP-HPLC-ELSD and RP-HPLC-ESI⁺ qTOF mass spectrometry. The solid mainly contained endo-BCN-PEG₃₆-[MAL-S]-DUPA (calculated monoisotopic mass: 3328.69 Da; measured: 3328.71 Da).

### Step 4. Synthesis of Me-LPEI-l-[N₃:BCN]-PEG₃₆-DUPA (Compound 81)

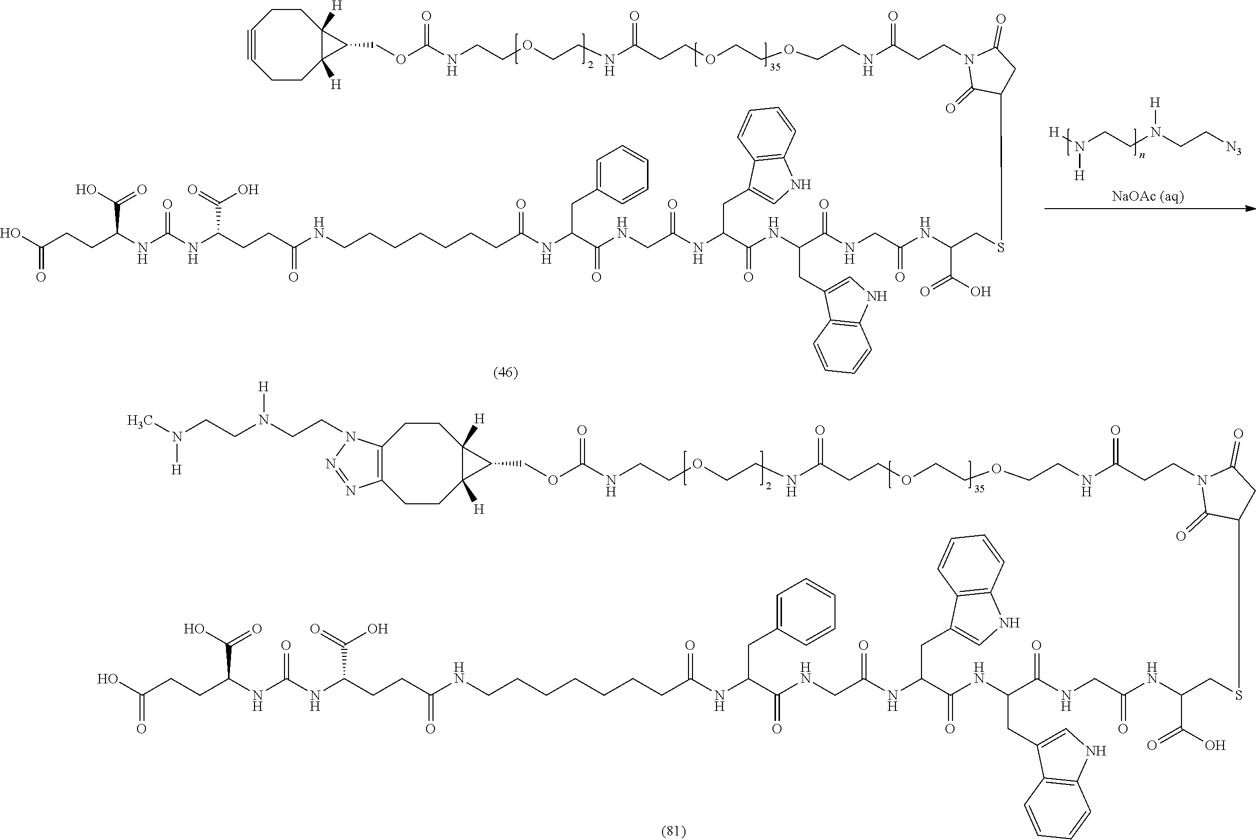

Me-LPEI-N₃ solution (4.7 μmol, 1.0 eq) in acetate buffer (50 mM, 3.2 mL, pH 4.0) was slowly added to a solution of endo-BCN-PEG₃₆-[MAL-S]-DUPA (Compound 46, 3.3 μmol, 0.7 eq, 1.1 mM) in acetate buffer (50 mM, 3.0 mL, pH 4.0). The mixture was shaken for about 45 hrs at room temperature on a Stuart rotator and protected from light. To the reaction mixture were added acetonitrile (0.66 mL) and TFA (70 μL) and the resultant mixture was purified using RP-C₁₈ preparative chromatography. Me-LPEI-l-[N₃:BCN]-PEG₃₆-DUPA (Compound 81) was lyophilized to give 55 mg lyophilized product and characterized by analytical RP-C₈ HPLC, copper assay and spectrophotometry at 280 nm for determination of the DUPA content. The product was found to have a weight percentage in LPEI of 28% w/w and a LPEI to DUPA ratio of 1/0.90.

### Step 5. Preparation of Me-LPEI-l-[N₃:BCN]-PEG₃₆-DUPA (Compound 81) HEPES Salt

24.8 mg of Me-LPEI-l-[N₃:BCN]-PEG₃₆-DUPA (Compound 81) TFA salt (w_{LPE}i=28%, 6.9 mg in total LPEI) were dissolved in 0.8 mL 20 mM HEPES pH 7.2. Two centrifugal filters (Amicon Ultra—0.5 mL, 10 kDa MWCO) were filled with 400 μL of Me-LPEI-l-[N₃:BCN]-PEG₃₆-DUPA solution each. They were centrifuged one time at 14000 g for 30 minutes and then three times after addition of 400 μL 20 mM HEPES, pH 7.2. About 253 μL of concentrated solution were recovered and supplemented with 2.5 mL 20 mM HEPES, pH 7.2. The concentration of the solution was determined by copper assay to be 2.3 mg/mL in total LPEI (LPEI/DUPA ratio=1/0.97).

### Example 22

### Synthesis of Me-LPEI-1-[N₃:DBCO]-PEG₃₆-DUPA (Compounds 82a and 82b)

Me-LPEI-l-[N₃:DBCO]-PEG₃₆-DUPA (82a and 82b) was synthesized according to the schemes below. In a first step, DBCO-PEG₃₆-MAL (Compound 36) was coupled to DUPA-Aoc-Phe-Gly-Trp-Trp-Gly-Cys (Compound 12; SEQ ID NO:4) by a Michael addition to produce DBCO-PEG₃₆-DUPA (Compound 37). In a next step, DBCO-PEG₃₆-DUPA (Compound 37) was coupled to Me-LPEI-N₃ by a [2+3]cycloaddition to produce Me-LPEI-l-[N₃:DBCO]-PEG₃₆-DUPA as a mixture of regioisomers 82a and 82b.

### Step 1: Synthesis of DBCO-PEG₃₆-DUPA (Compound 37)

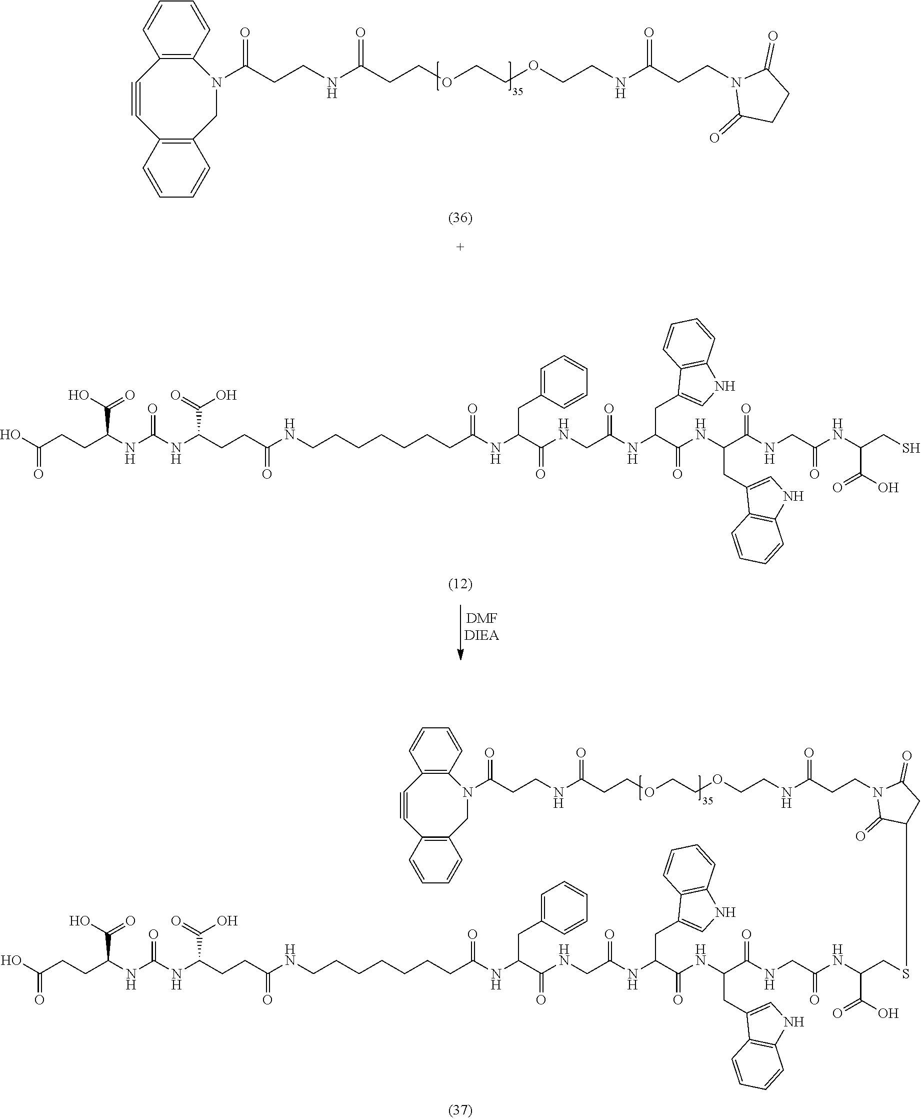

A solution of DBCO-PEG₃₆-MAL (Compound 36, 10 μmol, 1.0 eq, 49 mM) in DMF (0.20 mL) was mixed with a solution of DUPA-Aoc-Phe-Gly-Trp-Trp-Gly-Cys (Compound 12; SEQ ID NO:4) (10 μmol, 1.0 eq, 82 mM) (151 μL) and DIEA (3 μL, 20 μmol, 2.0 eq) in DMF. The mixture was shaken for about 30 minutes at room temperature on a Stuart rotator and protected from light and the reaction was monitored by RP-C₈ HPLC. The resultant mixture was purified using RP-C₁₈ preparative chromatography and pooled fractions containing DBCO-PEG₃₆-DUPA were lyophilized to give 20 mg of DBCO-PEG₃₆-DUPA (Compound 37). A sample was analyzed by analytical RP-HPLC-ELSD and HPLC-ESI⁺ qTOF mass spectrometry (calculated monoisotopic mass: 3280.61 Da; measured: 3280.64 Da)

### Step 2: Synthesis of Me-LPEI-l-[N₃:DBCO]-PEG₃₆-DUPA (Compounds 82a and 82b)

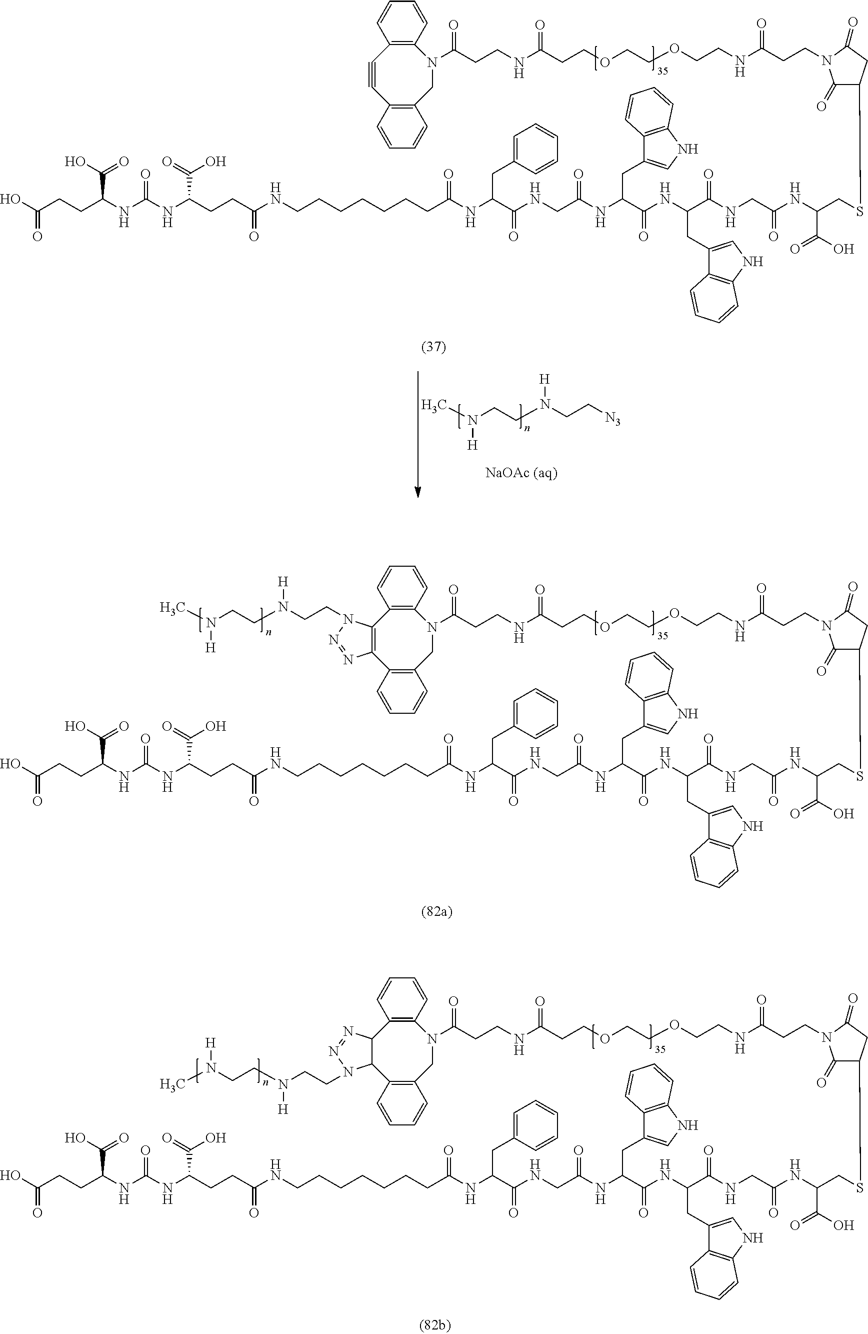

Me-LPEI-N₃ solution (4.5 μmol, 1.3 mM, 1.0 eq) in acetate buffer (50 mM, 3.2 mL, pH 4.0) was slowly added to a solution of DBCO-PEG₃₆-DUPA (Compound 37, 6.6 μmol, 1.5 eq, 2.2 mM) in acetate buffer (50 mM, 3.0 mL, pH 4.0). The mixture was shaken for about 20 hrs at room temperature on a Stuart rotator and protected from light. To the reaction mixture were added acetonitrile (0.70 mL) and TFA (70 μL). The resultant mixture was purified using RP-C₁₈ preparative chromatography and pooled fractions containing Me-LPEI-l-[N₃:DBCO]-PEG₃₆-DUPA (Compounds 82a and 82b) were lyophilized to give 70 mg lyophilized product and characterized by RP-C₈ HPLC, copper assay and spectrophotometry at 280 nm for determination of the DUPA content. The product was found to have a weight percentage in LPEI of 28% w/w and a LPEI to DUPA ratio of 1/1.17.

### Step 3. Preparation of Me-LPEI-l-[N₃:DBCO]-PEG₃₆-DUPA (Compounds 82a and 82b) HEPES Salt

25.0 mg of Me-LPEI-l-[N₃:DBCO]-PEG₃₆-DUPA (Compounds 82a and 82b) TFA salt (w_{LPEI}=28%, 7.0 mg in total LPEI) were dissolved in 0.8 mL 20 mM HEPES pH 7.2. The pH was adjusted to pH 7.2. Two centrifugal filters (Amicon Ultra—0.5 mL, 10 kDa MWCO) were filled with 400 μL of Me-LPEI-l-[N₃:DBCO]-PEG₃₆-DUPA solution each. They were centrifuged one time at 14000 g for 30 minutes and then three times after addition of 400 μL 20 mM HEPES, pH 7.2. About 254 μL of the concentrated solution were recovered after buffer exchange and were supplemented with 2.5 mL 20 mM HEPES, pH 7.2. The concentration of the solution was determined by copper assay to be 2.3 mg/mL in total LPEI and an LPEI to DUPA molar ratio of =1/1.19 was determined.

### Example 23

### Synthesis of LPEI-1-[N₃:CliCr®]-PEG₃₆-DUPA (Compound 83)

LPEI-l-[N₃:CliCr®]-PEG₃₆-DUPA (Compound 83) was synthesized according to the schemes below. In a first step, CliCr®-beta-Ala-NH₂ (Compound 84) was coupled to HOOC-PEG₃₆-MAL (Compound 34) to produce CliCr®-PEG₃₆-MAL (Compound 85). Subsequently, CliCr®-PEG₃₆-MAL (Compound 85) was coupled to DUPA-Aoc-Phe-Gly-Trp-Trp-Gly-Cys (Compound 12; SEQ ID NO:4) by a Michael addition to produce CliCr®-PEG₃₆-DUPA (Compound 86). In a final step, CliCr®-PEG₃₆-DUPA (Compound 86) was reacted with LPEI-N₃ in a [2+3]cycloaddition reaction to produce LPEI-l-[N₃:CliCr®]-PEG₃₆-DUPA (Compound 83).

### Step 1: Synthesis of HOOC-PEG₃₆-MAL (Compound 34)

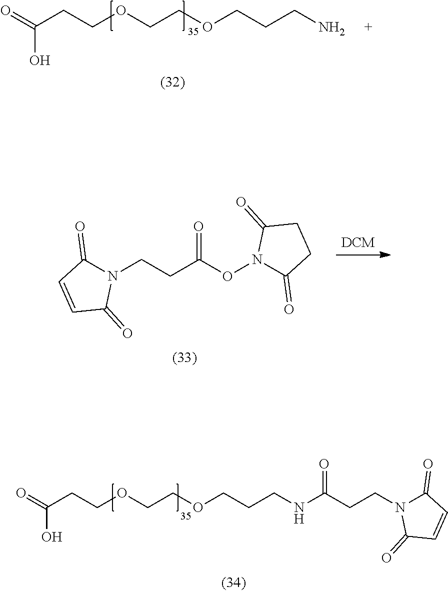

A solution of HOOC-PEG₃₆-NH₂ (Compound 32, 94 μmol, 1.0 eq, 234 mM) in DCM (0.40 mL) was mixed with a solution of N-succinimidyl 3-maleimidopropionate (Compound 33, 85 μmol, 0.9 eq, 184 mM) in DCM (0.83 mL). The reaction mixture was shaken for one hour on a Stuart rotator at room temperature and the reaction was monitored by RP-C₈ HPLC. An additional amount of N-succinimidyl 3-maleimidopropionate (220 μL, 23 μmol, 0.25 eq) was added. After a total of 3.5 hours mixing, 4.5 mL of cold diethyl ether were added to induce precipitation followed by centrifugation. The precipitate was washed with 4.5 mL cold diethyl ether and 183 mg of HOOC-PEG₃₆-MAL (Compound 34) were recovered (calculated monoisotopic mass: 1825.03 Da; measured: 1825.02 Da).

### Step 2: Synthesis of CliCr®-PEG₃₆-MAL (Compound 85)

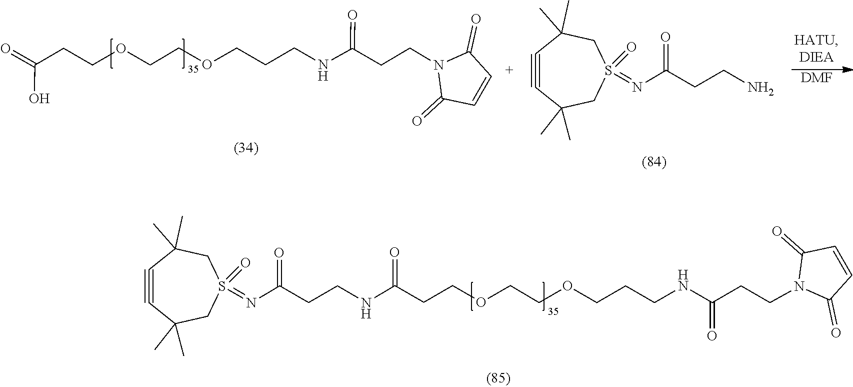

A solution of HOOC-PEG₃₆-MAL (Compound 34, 17 μmol, 1.0 eq, 42 mM) in DMF (0.40 mL) was mixed with a solution of HATU (17 μmol, 1.0 eq, 89 mM) and DIEA (6 μL, 34 μmol, 2.0 eq) in DMF (191 μL). A solution of CliCr®-beta-Ala-NH₂ (Compound 84, 20 μmol, 1.2 eq, 163 mM) in DMF (123 μL) was then added. The mixture was shaken for about 30 minutes at room temperature on a Stuart rotator and the reaction was monitored by RP-C₈ HPLC. The resultant mixture was purified using RP-C₁₈ preparative chromatography and pooled fractions containing CliCr®-PEG₃₆-MAL were lyophilized to give 11 mg of CliCr®-PEG₃₆-MAL (Compound 85). A sample was analyzed by analytical RP-HPLC ELSD and HPLC-ESI⁺ qTOF mass spectrometry (calculated monoisotopic mass: 2077.15 Da; measured: 2077.17 Da).

### Step 3: Synthesis of CliCr®-PEG₃₆-DUPA (Compound 86)

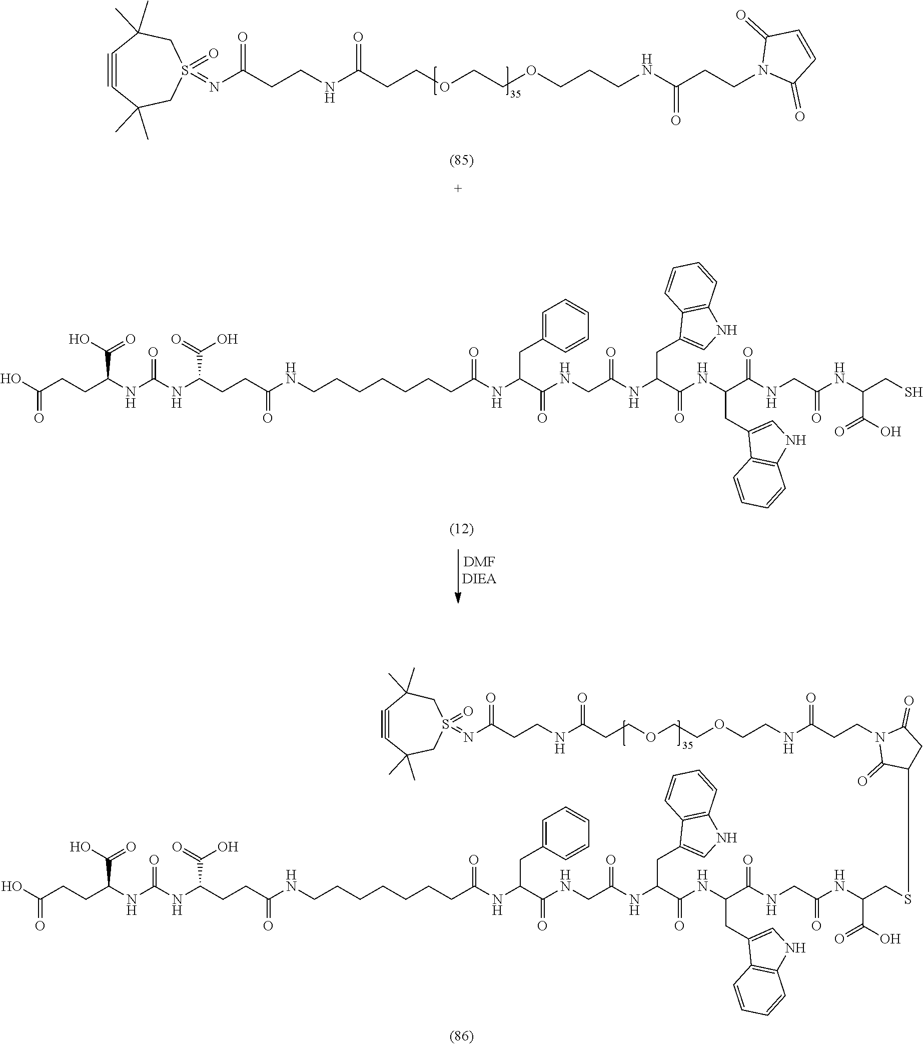

A solution of CliCr®-PEG₃₆-MAL (Compound 85, 5.3 μmol, 1.0 eq, 13 mM) in DMF (0.40 mL) was mixed with a solution of DUPA-Aoc-Phe-Gly-Trp-Trp-Gly-Cys (Compound 12; SEQ ID NO:4) (5.3 μmol, 1.0 eq, 44 mM) (120 μL) and DIEA (1.8 μL, 11 μmol, 2.0 eq) in DMF. The mixture was shaken for about 30 minutes at room temperature on a Stuart rotator and the reaction was monitored by RP-C₈ HPLC. The resultant mixture was purified using RP-C₁₈ preparative chromatography and pooled fractions containing CliCr®-PEG₃₆-DUPA were lyophilized to give 11 mg of CliCr®-PEG₃₆-DUPA (Compound 86). A sample was analyzed by analytical RP-HPLC ELSD and HPLC-ESI⁺ qTOF mass spectrometry (calculated monoisotopic mass: 3274.63 Da; measured: 3274.66 Da).

### Step 4: Synthesis of LPEI-l-[N₃:CliCr®]-PEG₃₆-DUPA (Compound 83)

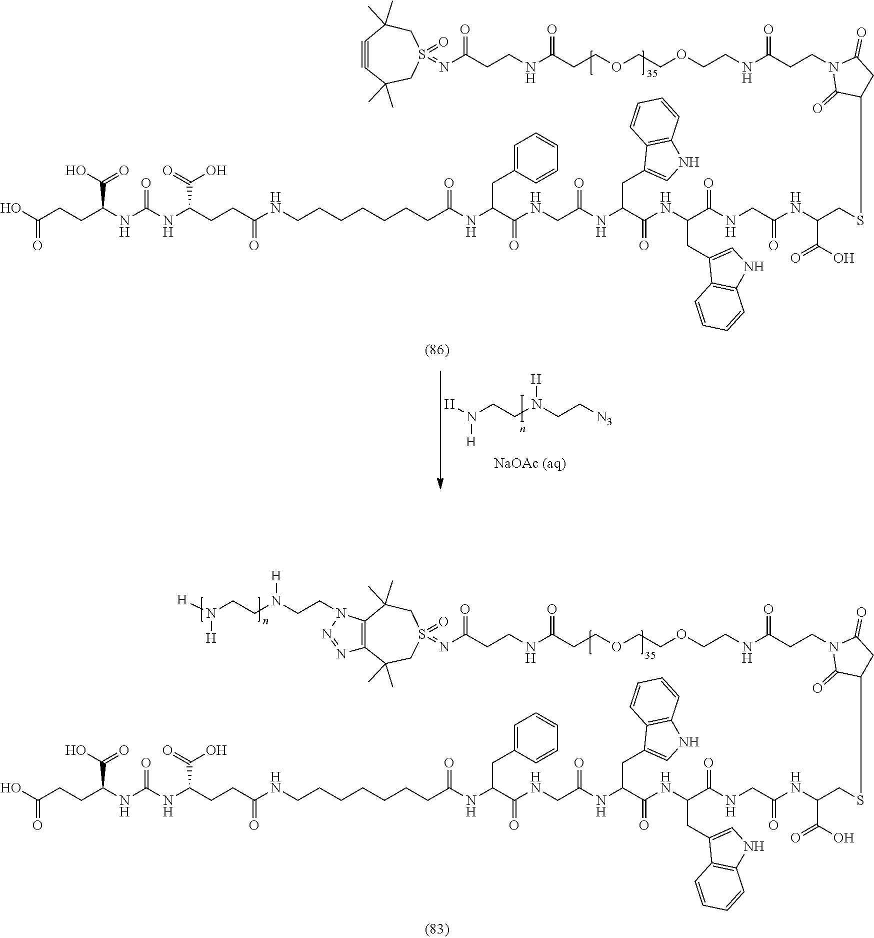

LPEI-N₃ solution (4.1 μmol, 0.84 mM, 1.0 eq) in acetate buffer (50 mM, 5.0 mL, pH 4.0) was slowly added to a solution of CliCr®-PEG₃₆-DUPA (Compound 86, 3.9 μmol, 1.0 eq, 3.9 mM) in acetate buffer (50 mM, 1.0 mL, pH 4.0). The mixture was shaken for about 3 hrs at room temperature on a Stuart rotator. To the reaction mixture were added acetonitrile (0.67 mL) and TFA (67 μL). The resultant mixture was purified using RP-C₁₈ preparative chromatography and pooled fractions containing LPEI-l-[N₃:CliCr®]-PEG₃₆-DUPA (Compound 83) were lyophilized to give 85 mg lyophilized product and characterized by RP-C₈ HPLC, copper assay and spectrophotometry at 280 nm for determination of the DUPA content. The product was found to have a weight percentage in LPEI of 29% w/w and a LPEI to DUPA ratio of 1/1.0.

### Step 5: Preparation of LPEI-l-[N₃:CliCr®]-PEG₃₆-DUPA (Compound 83) HEPES Salt

27.0 mg of LPEI-l-[N₃:CliCr®]-PEG₃₆-DUPA (Compound 83) TFA salt (w_{LPEI}=29%, 7.8 mg in total LPEI) were dissolved in 0.8 mL 20 mM HEPES pH 7.2. The pH was adjusted to pH 7.2. Two centrifugal filters (Amicon Ultra-0.5 mL, 10 kDa MWCO) were filled with 400 μL of LPEI-l-[N₃:CliCr®]-PEG₃₆-DUPA solution each. They were centrifuged one time at 14000 g for 30 minutes and then three times after addition of 400 μL 20 mM HEPES, pH 7.2. About 249 μL of the concentrated solution were recovered after buffer exchange and were supplemented with 3.0 mL 20 mM HEPES, pH 7.2. The concentration of the solution was determined by copper assay to be 2.0 mg/mL in total LPEI and an LPEI to DUPA molar ratio of=1/1.0 was determined.

### Example 24

### Polyplex Formation, Polyplex Sizing and Zeta Potential Measurements

General Procedure for Polyplex Formation with mRNA.

For the preparation of preferred polyplexes, the respective triconjugates were complexed with selected mRNAs at various N/P ratios in 5% glucose (wt/vol) or HBS (Hepes-buffered saline pH 7.2). Nitrogen to phosphorus (N/P) ratios were calculated based on the nitrogen content in the LPEI portion of the used triconjugates and the phosphorous content in the mRNA. The concentrations of the triconjugate such as LPEI-l-[N₃:DBCO]-PEG₃₆-DUPA or LPEI-l-[N₃:DBCO]-PEG₃₆-hEGF (expressed as total LPEI in mg/mL) at each mRNA concentration and N/P ratio are summarized in the table below:

| mRNA concentration | N/P ratio | | | | | |
|---|---|---|---|---|---|---|
| (mg/mL) | 3 | 4 | 5 | 6 | 8 | 12 |
| 0.10 | 0.039 | 0.052 | 0.065 | 0.078 | 0.104 | 0.156 |
| 0.02 | 0.0078 | 0.0104 | 0.013 | 0.0156 | 0.0208 | 0.0312 |

Hereby, stock solutions of triconjugates such as LPEI-l-[N₃:DBCO]-PEG₃₆-DUPA or LPEI-l-[N₃:DBCO]-PEG₃₆-hEGF and mRNA were diluted with 5% glucose or HBS to the appropriate concentrations for the selected N/P ratio prior to mixing. The diluted triconjugate solution was added to an equal volume of nucleic acid solution to a final concentration of 0.1-0.02 mg/mL of nucleic acid in the polyplex preparation and mixed vigorously. The mixture was incubated at RT for 30 min for polyplex formation prior to use. In an analogous manner, polyplexes with polyanions such as poly(Glu) were prepared. The polyplexes were typically further characterized with respect to particle size distribution and ζ-potential.

Physico-chemical characterization by Dynamic Light Scattering (DLS) of polyplexes comprising various mRNA and various inventive triconjugates are shown in Tables 7 to 14.

TABLE 7
Particle size distribution and polydispersity data by DLS
for polyplexes comprising various mRNA and the triconjugate
LPEI-l-[N₃:DBCO]-PEG₃₆-hEGF
at the indicated N/P ratios.

| | DLS: Z-Average (d, nm) | | | DLS: PDI (Polydispersity Index) | | |
|---|---|---|---|---|---|---|
| | N/P ratio | | | N/P ratio | | |
| | 3 | 6 | 12 | 3 | 6 | 12 |
| mRNA | Mean | Mean | Mean | Mean | Mean | Mean |
| IL-2 | 109.1 | 116.8 | 126.5 | 0.165 | 0.208 | 0.242 |
| (725 NT)* | | | | | | |
| IFN-γ | 120.3 | 118.9 | 130.2 | 0.181 | 0.215 | 0.195 |
| (762 NT)* | | | | | | |

For all measurements, the viscosity value of 1.078 mPa · s was used except for the measurements marked with an asterisk (*), for which the viscosity value of 0.98 mPa · s was used.

TABLE 8
Particle size distribution and polydispersity data by DLS
for polyplexes comprising various mRNA and the triconjugate
LPEI-l-[N₃:DBCO]-PEG₃₆-DUPA (Compounds 12a
and 12b) at 0.1 mg/mL in 5% glucose and
the indicated N/P ratios.

| | DLS: Z-Average (d, nm) | | | DLS: PDI (Polydispersity Index) | | |
|---|---|---|---|---|---|---|
| | N/P ratio | | | N/P ratio | | |
| | 4 | 6 | 12 | 4 | 6 | 12 |
| mRNA | Mean | Mean | Mean | Mean | Mean | Mean |
| IL-2 | 152.4 | 127.1 | 140.9 | 0.042 | 0.156 | 0.204 |
| (725 NT)* | | | | | | |
| Luc | 132.4 | 101.6 | 114.5 | 0.120 | 0.147 | 0.150 |
| (1929 NT)* | | | | | | |
| IFN-β | 103.4 | 98.67 | 123.4 | 0.173 | 0.214 | 0.236 |
| (1047 NT) | | | | | | |
| Diphtheria toxin | 103.4 | 106.6 | 118.7 | 0.197 | 0.194 | 0.228 |
| (1068 NT) | | | | | | |

For all measurements, the viscosity value of 1.078 mPa · s was used except for the measurements marked with an asterisk (*), for which the viscosity value of 0.98 mPa · s was used.

TABLE 9
Particle size distribution and polydispersity data by DLS
for polyplexes comprising the indicated mRNA and the triconjugate
LPEI-l-[N3:DBCO]-PEG24-Folate at the indicated N/P ratios.

| | DLS: Z-Average (d, nm) | | | DLS: PDI (Polydispersity Index) | | |
|---|---|---|---|---|---|---|
| | N/P ratio | | | N/P ratio | | |
| | 5 | 8 | 12 | 5 | 8 | 12 |
| mRNA | Mean | Mean | Mean | Mean | Mean | Mean |
| Renilla Luc | 106.6 | 96.7 | 75.1 | 0.096 | 0.155 | 0.208 |
| (1212 NT) | | | | | | |
| Epo | 91.4 | 80.4 | nd | 0.132 | 0.135 | nd |
| (858 NT) | | | | | | |

nd = not determined

TABLE 10
Particle size distribution and polydispersity data by DLS
for polyplexes comprising the indicated mRNA and the
triconjugate LPEI-l-[N₃:DBCO]-PEG₃₆-(GalNAc)₃
at the indicated N/P ratios.

| | DLS: Z-Average (d, nm) | | | | DLS: PDI (Polydispersity Index) | | | |
|---|---|---|---|---|---|---|---|---|
| | N/P ratio | | | | N/P ratio | | | |
| | 3 | 5 | 8 | 12 | 3 | 5 | 8 | 12 |
| mRNA | Mean | Mean | Mean | Mean | Mean | Mean | Mean | Mean |
| Renilla Luc | 77.1 | 80.0 | 86.9 | 81.9 | 0.167 | 0.199 | 0.180 | 0.190 |
| (1212 NT) | | | | | | | | |
| Epo | 93.5 | 82.7 | 83.6 | 90.5 | 0.151 | 0.153 | 0.164 | 0.200 |
| (858 NT) | | | | | | | | |

nd = not determined

TABLE 11
ζ-potentials of polyplexes comprising the indicated
mRNA and the triconjugate LPEI-l-[N₃:DBCO]-PEG₃₆-hEGF
at the indicated N/P ratios.

| | Zeta potential (mV) | | |
|---|---|---|---|
| | N/P ratio | | |
| | 3 | 6 | 12 |
| mRNA | Mean | Mean | Mean |
| IL-2 (725 NT)* | 30.7 | 44.3 | 49.3 |
| IFN-γ (762 NT)* | 35.5 | 53.8 | 56.6 |

For all measurements, the viscosity value of 0.98 mPa · s was used.

TABLE 12
ζ-potentials of polyplexes polyplexes comprising
various mRNA and the triconjugate LPEI-l-[N₃:DBCO]-
PEG₃₆-DUPA (Compounds 12a and 12b) at 0.1 mg/mL
in 5% glucose and the indicated N/P ratios.

| | Zeta potential (mV) | | |
|---|---|---|---|
| | N/P ratio | | |
| | 4 | 6 | 12 |
| mRNA | Mean | Mean | Mean |
| IL-2 (725 NT)* | 19.3 | 31.4 | 43.5 |
| Luc (1929 NT)* | 27.9 | 44.7 | 43.0 |
| IFN-β (1047 NT) | 45.44 | 47.09 | 46.8 |
| Diphtheria toxin (1068 NT) | 44.47 | 46.99 | 48.86 |

For all measurements, the viscosity value of 1.078 mPa · s was used except for the measurements marked with an asterisk (*), for which the viscosity value of 0.98 mPa · s was used.

TABLE 13
ζ-potentials of polyplexes comprising various mRNA and the triconjugate
LPEI-l-[N3:DBCO]-PEG24-Folate at the indicated N/P ratios.

| | Zeta potential (mV) | | |
|---|---|---|---|
| | N/P ratio | | |
| | 5 | 8 | 12 |
| mRNA | Mean | Mean | Mean |
| Renilla Luc (1212 NT) | 25.9 | 43.0 | 41.2 |
| Epo (858 NT) | 32.6 | 36.9 | nd |

nd = not determined

TABLE 14
ζ-potentials of polyplexes comprising various
mRNA and the triconjugate LPEI-l-[N₃:DBCO]-
PEG₃₆-(GalNAc)₃ at the indicated N/P ratios.

Zeta potential (mV)
N/P ratio

| | 3 | 5 | 8 | 12 |
|---|---|---|---|---|
| mRNA | Mean | Mean | Mean | Mean |
| Renilla Luc (1212 NT) | 21.2 | 32.8 | 34.4 | 29.8 |
| Epo (858 NT) | 21.7 | 24.0 | 32.1 | 33.3 |

nd = not determined

In all tested samples mean Z-average diameter in the range between 75 nm and 153 nm was observed and particles were found to be monodispersed (PDI<0.3).

In all samples positive mean ζ-potential in the range of 19 mV and 57 mV was observed.

### Example 25

Selective Delivery of mRNA Encoding Luciferase by Inventive Polyplexes Results in High Expression of Luciferase in Various EGFR Overexpressing Cells

Polyplexes comprising Fluc mRNA and LPEI-l-[N₃:DBCO]-PEG₃₆-hEGF (i.e., Compounds 70a and 70b) were generated by complexing the Fluc mRNA in HEPES-buffered saline (HBS: 20 mM HEPES, 150 mM NaCl, pH 7.2) at 0.02 mg/ml with the triconjugate LPEI-l-[N₃:DBCO]-PEG₃₆-hEGF at N/P ratios of 4, 6 and 12 (where N=nitrogen from LPEI and P=phosphate of mRNA). To allow complete formation of the polyplex particles, i.e., LPEI-l-[N₃:DBCO]PEG₃₆-hEGF:[Fluc mRNA], the samples were incubated for 30 min at room temperature.
1. Renca parental cells (mouse renal carcinoma, human EGFR negative); and RencaEGFR M1 H cells (derivate of Renca parental engineered to overexpress human EGFR) were cultured according to manufacturer's protocol. Renca (parental) cells were cultured in RPMI medium supplemented with 10% fetal bovine serum (FBS), 104 U/L penicillin, 10 mg/L streptomycin at 37° C. in 5% CO₂. 400 μg/ml of G418 were added to the medium of RencaEGFR M1 H cells. 15,000 cells/well RencaEGFR M1 H cells, 10,000 cells/well Renca parental cells were seeded in triplicates at 90 μl into 96 well white plates (Greiner) and 96 well transparent plates (Nunc). Cells were transfected with 0.125-1 mg/ml of LPEI-l-[N₃:DBCO]PEG₃₆-hEGF:[Fluc mRNA].
2. B16F10-hEGFR cells which overexpresses human EGFR (the mouse melanoma cell line B16F10, was stably transfected with a human EGFR plasmid encoding human EGFR) were cultured in DMEM medium supplemented with 10% fetal bovine serum (FBS), 104 U/L penicillin, 10 mg/L streptomycin at 37° C. in 5% CO₂. 25 μg/ml of Zeocin was added to the medium. The indicated cell numbers were seeded in triplicates at 90 μl into 96 well white plates (Greiner). Cells were transfected with 0.125-1.0 mg/ml of LPEI-l-[N₃:DBCO]PEG₃₆-hEGF:[Fluc mRNA].

Luciferase activity was measured with OneGloX assay (Promega) at the indicated time after the treatment. Luminescence measurements were performed using a Luminoskan Ascent Microplate Luminometer (Thermo Scientific). Values, in Arbitrary Units (AU), are presented as the mean and standard deviation of luciferase activity from the triplicate samples.

Cell survival was measured by means of a colorimetric assay using methylene blue assay. Briefly, the cells were fixed with 2.5% glutaraldehyde in PBS (pH 7.4), washed with deuterium depleted water (DDW), and then stained with a 1% (wt/vol) solution of methylene blue in borate buffer for one hour. Thereafter, the stain was extracted with 0.1 M HCl and the optical density of the stain solution was read at 630 nm on a microplate reader (Synergy H1, Biotek). Luminescence and cell survival were measured 24 hrs after the treatment.

Physicochemical characterization of the LPEI-l-[N₃:DBCO]PEG₃₆-hEGF:[Fluc mRNA] polyplexes was measured using DLS in 50 mM acetate buffer, 5% glucose at pH 4.3, at N/P ratios of 3, 4, 5, and 6. A summary of physicochemical measurements is given in Table 15. The z-average diameter ranged between 95 nm and 127 nm with a polydispersity index (PDI) of 0.134-0.209. The ζ-potential range measured by ELS was 29.7-45.6 mV.

TABLE 15
Physicochemical Characterization of polyplex
LPEI-l-[N₃:DBCO]PEG₃₆-hEGF:FLuc mRNA
in in 50 mM acetate buffer, 5% glucose
at pH 4.3 at N/P ratios 3, 4, 5, 6

| N/P | 6 | 5 | 4 | 3 |
|---|---|---|---|---|
| Fluc mRNA (mg/mL) | 0.1 | 0.1 | 0.1 | 0.1 |
| Average diameter (nm) | 125.1 | 95 | 119.2 | 127.9 |
| PDI | 0.207 | 0.209 | 0.196 | 0.134 |
| ζ-potential | 45.6 | 42.4 | 29.7 | 41 |

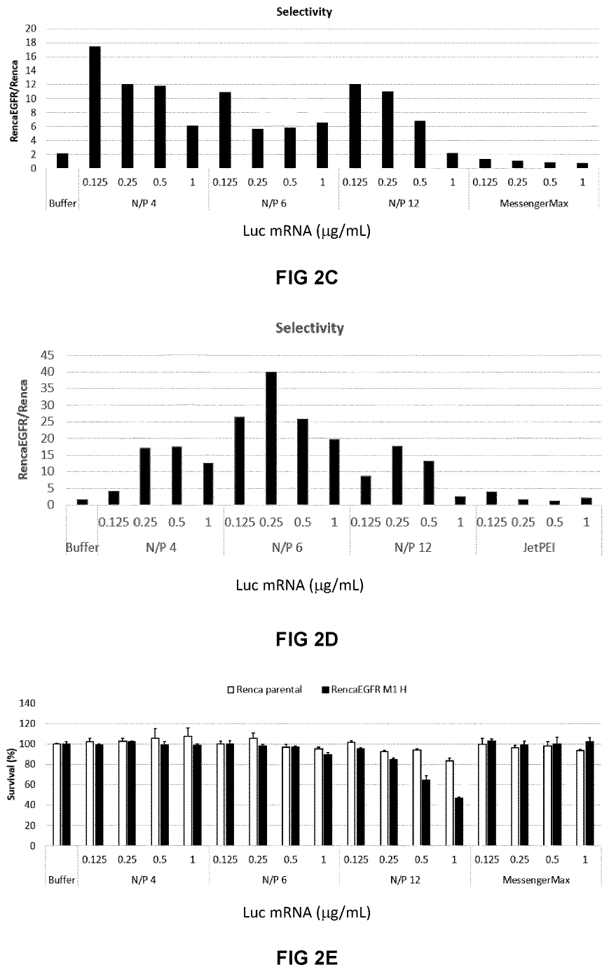

FIG. 2A is a plot of luminescence (AU) in Renca parental cells and Renca EGFR M1 H cells treated with LPEI-l-[N₃:DBCO]PEG₃₆-hEGF:[Fluc mRNA] compared to the control delivery vehicle Messenger MAX. The luminescence was measured at N/P ratios of 4, 6 and 12, and at concentrations from 0.125 to 1.0 μg/mL of LPEI-l-[N₃:DBCO]PEG₃₆-hEGF:[Fluc mRNA] and lipofectamine messenger MAX at 24 hours after treatment.

FIG. 2B is a plot of luminescence (AU) in Renca parental cells and Renca EGFR M1 H cells treated with LPEI-l-[N₃:DBCO]PEG₃₆-hEGF:[Fluc mRNA] compared to the control delivery vehicle jetPEI. The luminescence was measured at N/P ratios of 4, 6 and 12, and at concentrations from 0.125 to 1.0 μg/mL of LPEI-l-[N₃:DBCO]PEG₃₆-hEGF:[Fluc mRNA] and jetPEI at 24 hours after treatment.

FIG. 2C is a plot of the ratio of luminescence (AU) between Renca parental cells and Renca EGFR M1 H cells treated with LPEI-l-[N₃:DBCO]PEG₃₆-hEGF:[Fluc mRNA]. The luminescence was measured at N/P ratios of 4, 6 and 12, and at concentrations from 0.125 to 1.0 μg/mL of LPEI-l-[N₃:DBCO]PEG₃₆-hEGF:[Fluc mRNA] and lipofectamine Messenger MAX at 24 hours after treatment. The ratio was calculated by dividing the luminescence signal from RencaEGFR M1 H cells by the luminescence signal from Renca parental cells.

FIG. 2D is a plot of the ratio of luminescence (AU) between Renca parental cells and Renca EGFR M1 H cells treated with LPEI-l-[N₃:DBCO]PEG₃₆-hEGF:[Fluc mRNA] with jetPEI as a comparison delivery vehicle. The luminescence was measured at N/P ratios of 4, 6 and 12, and at concentrations from 0.125 to 1.0 mg/mL of LPEI-l-[N₃:DBCO]PEG₃₆-hEGF:[Fluc mRNA] and jetPEI at 24 hours after treatment. The ratio was calculated by dividing the luminescence signal from RencaEGFR M1 H cells by the luminescence signal from Renca parental cells.

FIGS. 2A-2D show that selective mRNA delivery to RencaEGFR M1 H cells over Renca parental cells was achieved using LPEI-l-[N₃:DBCO]PEG₃₆-hEGF:[Fluc mRNA] at N/P ratios of 4-12. In contrast, the non-targeted delivery vehicles Lipofectamine messenger MAX and jetPEI did not show selective mRNA delivery to either cell line. In both cases superiority over non-targeted delivery systems was demonstrated across all N/P ratios.

FIG. 2E shows that the LPEI-l-[N₃:DBCO]PEG₃₆-hEGF:[Fluc mRNA] polyplexes were not cytotoxic at N/P 4 and 6.

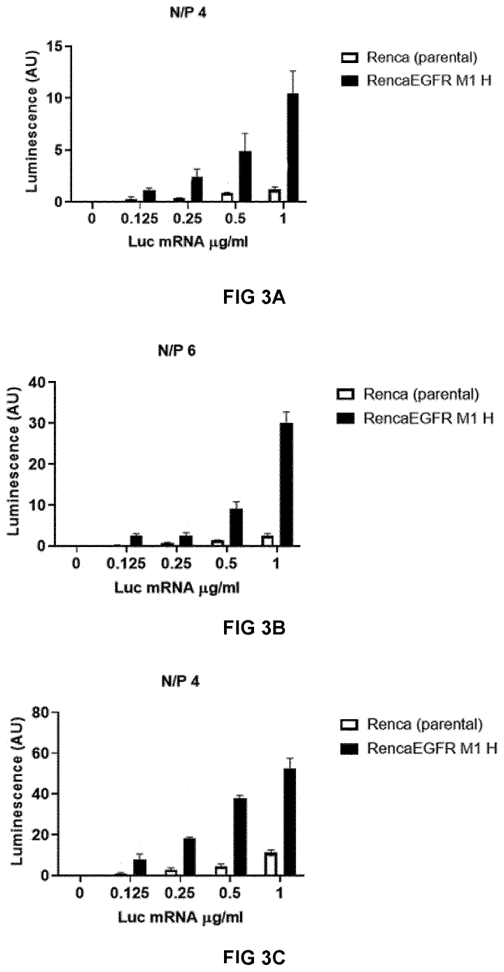

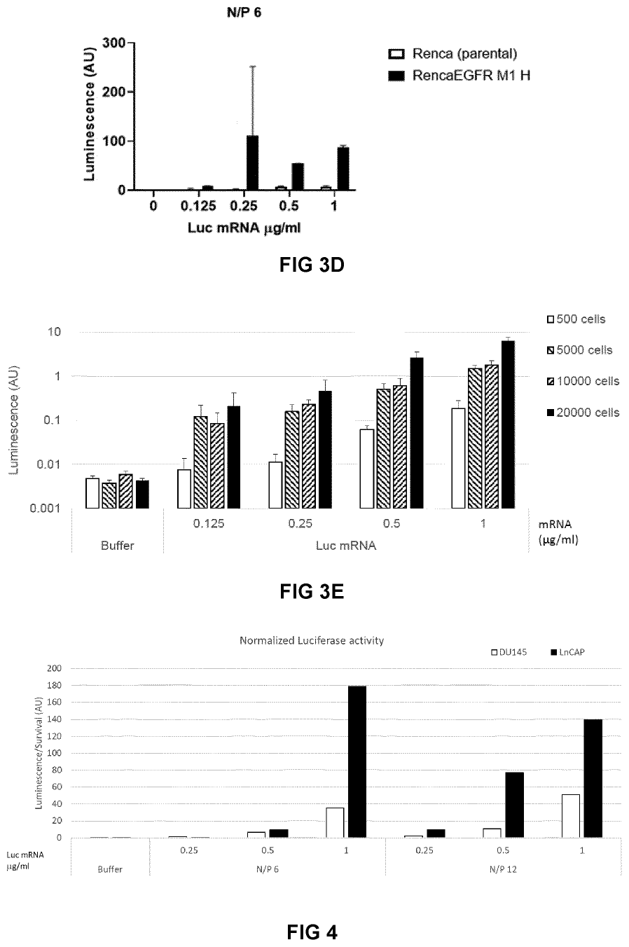

FIGS. 3A-3D show relative luminescence (AU) in Renca parental cells and Renca EGFR M1 H cells treated with LPEI-l-[N₃:DBCO]PEG₃₆-hEGF:[Fluc mRNA] at 6 and 22 hours after delivery. Selective delivery and expression were achieved at 6 hours, with peak at 22 hours.

FIG. 3A shows relative luminescence (AU) in Renca parental cells and Renca EGFR M1 H cells treated with LPEI-l-[N₃:DBCO]PEG₃₆-hEGF:[Fluc mRNA] at 6 hours after treatment at an N/P of 4.

FIG. 3B shows relative luminescence (AU) in Renca parental cells and Renca EGFR M1 H cells treated with LPEI-l-[N₃:DBCO]PEG₃₆-hEGF:[Fluc mRNA] at 6 hours after treatment at an N/P of 6.

FIG. 3C shows relative luminescence (AU) in Renca parental cells and Renca EGFR M1 H cells treated with LPEI-l-[N₃:DBCO]PEG₃₆-hEGF: [Fluc mRNA] at 22 hours after treatment at an N/P of 4.

FIG. 3D shows relative luminescence (AU) in Renca parental cells and Renca EGFR M1 H cells treated with LPEI-l-[N₃:DBCO]PEG₃₆-hEGF: [Fluc mRNA] at 22 hours after treatment at an N/P of 6.

FIG. 3E shows relative luminescence (AU) from B16F10-hEGFR cells treated with LPEI-l-[N₃:DBCO]PEG₃₆-hEGF:[Fluc mRNA]24 hours after treatment at an N/P ratio of 6 at varying cell numbers. mRNA delivery to B16F10-hEGFR cells was achieved using LPEI-l-[N₃:DBCO]PEG₃₆-hEGF:[Fluc mRNA] at an N/P ratio of 6. Significant increase in luminescence was obtained following transfection of 500 cells and up to 20,000 cells.

The provided data show that the inventive polyplexes are not only selectively delivering mRNA to the desired cells which overexpress the target receptor, but furthermore show delivery of the mRNA payload in a manner that significant functional expression of the encoded protein takes place. In addition and remarkably, efficient expression were obtained using the inventive polyplexes even in low number of transfected cells.

### Example 26

Selective Delivery of mRNA Encoding Luciferase Using the Inventive Polyplexes Results in High Expression of Luciferase in PSMA Overexpressing Cells

LPEI-l-[N₃:DBCO]-PEG₃₆-DUPA:Luc mRNA polyplexes were formulated in HBS at N/P ratios of 6 and 12. Prostate cancer cell lines with differential expression of PSMA (DU145: low PSMA expression; LNCaP: high PSMA expression) were treated with the polyplexes. Luminescence was measured 24 hours after the transfection and detected using Luminoskan Ascent Microplate Luminometer (Thermo Scientific).

In detail, 15,000 cells of humane prostate cancer cell lines LNCaP (high expression of PSMA) and DU145 (low expression of PSMA) were seeded into 96 well plates in triplicates and grown over night. Luc mRNA (Trilink Biotechnologies, L-7602 comprising SEQ ID NO:15 (mRNA LUC ORF) was formulated with LPEI-l-[N₃:DBCO]-PEG₃₆-DUPA in HBS (HEPES Buffered Saline, 20 mM HEPES, 150 mM NaCl, pH 7.3). The mRNA was first diluted with HBS to 0.04 mg/ml for all N/P ratios. LPEI-l-[N₃:DBCO]-PEG₃₆-DUPA was diluted with HBS to 0.0312 mg/ml (N/P 6) and 0.0624 mg/ml (N/P 12). The diluted LPEI-l-[N₃:DBCO]-PEG₃₆-DUPA was added to the diluted mRNA and mixed by pipetting and were incubated for 30 minutes at room temperature to form polyplex. The final concentration of mRNA and LPEI-l-[N₃:DBCO]-PEG₃₆-DUPA in the polyplexes at the indicated N/P ratios are presented below:

| N/P ratio | 6 | 12 |
|---|---|---|
| Conc of mRNA (mg/ml) | 0.02 | 0.02 |
| Conc of LPEI-l-[N₃:DBCO]-PEG₃₆-DUPA (mg/ml) | 0.0156 | 0.0312 |

The polyplexes were serially diluted and added to the cells (using 10× dilution) to obtain the indicated final concentrations of the mRNA (0.25, 0.5 and 1.0 μg/ml). Luciferase activity was measured 24 hours after the transfection with ONE-Glo™ EX Luciferase Assay System (Promega, Cat #E8130) and detected using Luminoskan Ascent Microplate Luminometer (Thermo Scientific).

Cell survival assay: Cell viability was measured by a colorimetric Methylene Blue assay. Briefly, the cells were fixed with 2.5% Glutaraldehyde in PBS (pH 7.4), washed with double distilled water, and then stained with a 1% (wt/vol) solution of methylene blue in borate buffer for 1 hour. The stain was extracted with 0.1 M HCl and the optical density of the stain solution was read at 630 nm in a microplate reader (Synergy H1, Biotek).

Selective delivery of LPEI-1-[N₃:DBCO]-PEG₃₆-DUPA:Luc mRNA resulted in high expression of Luciferase in LNCaP cells at N/P ratios of 6 and 12 (FIG. 4). In contrast, much lower expression of Luciferase was obtained in DU145 cells. These results demonstrate the selectivity of delivery of Luc mRNA to cancer cells with high expression of PSMA and efficient translation of a functional luciferase protein.

### Example 27

### Selective Delivery of Renilla Luciferase mRNA Using Inventive Polyplexes Results in High Expression of Luciferase in Folate Receptor-Overexpressing Cells

LPEI-l-[N₃:DBCO]-PEG₂₄-Folate: Renilla μLuc mRNA polyplexes were formulated in HBS at N/P ratios of 5, 8 or 12. Cancer cell lines with differential expression of folate receptor (MCF7: low folate receptor; SKOV3: high folate receptor expression) were treated with the polyplexes. Luminescence signal was examined 24 hours after the transfection by Renilla-Glo Luciferase Assay System (Promega, Cat #E2720).

In detail, SKOV3 cells (15,000 cells/well) and MCF7 cells (15,000 cells/well) were seeded into 96 well plates in quadruplicates and grown overnight. Renilla μLuciferase mRNA (Trilink Biotechnologies, L-7204 comprising SEQ ID NO:16 (mRNA Renilla μLuc ORF)) was formulated with LPEI-l-[N₃:DBCO]-PEG₂₄-Folate in HBS (Hepes Buffered Saline, 20 mM HEPES, 150 mM NaCl, pH 7.3). The mRNA was first diluted with HBS to 0.04 mg/ml for all N/P ratios. LPEI-l-[N₃:DBCO]-PEG₂₄-Folate was diluted with HBS to 0.026 mg/ml (N/P ratio 5), 0.042 mg/ml (N/P ratio 8) and 0.062 mg/ml (N/P ratio 12). The diluted LPEI-l-[N₃:DBCO]-PEG₂₄-Folate was added to the diluted mRNA and mixed by pipetting and incubated for 30 minutes at room temperature to form polyplexes. The final concentration of LPEI-l-[N₃:DBCO]-PEG₂₄-Folate and mRNA in the polyplexes were:

| N/P ratio | 5 | 8 | 12 |
|---|---|---|---|
| Conc of mRNA (mg/ml) | 0.02 | 0.02 | 0.02 |
| Conc of LPEI-l-[N₃:DBCO]-PEG₂₄-Folate (mg/ml) | 0.013 | 0.021 | 0.031 |

The polyplexes were serially diluted and added to the cells to obtain the indicated final concentrations of the mRNA (0.125, 0.25, 0.5 and 1.0 μg/mL). Luminescence produced by the expression of the Luciferase protein was measured after 24 hours using a Wallac Victor Light, 1420 μLuminescence Counter.

Selective delivery of LPEI-l-[N₃:DBCO]-PEG₂₄-Folate:Renilla μLuc mRNA resulted in high expression and activity of Renilla μLuciferase as indicated by high luminescence signal in folate receptor overexpressing cells (SKOV3) at all the tested N/P ratios in a dose-dependent manner (FIG. 5). In contrast, lower luminescence was detected in MCF7 cells. These results demonstrate the selectivity of delivery of Renilla μLuciferase mRNA to cancer cells with high expression of folate receptor and the efficient protein translation and activity.

### Example 28

Selective Delivery of Human IL-2 mRNA Using the Inventive Polyplexes Results in High Expression of Human IL-2 in EGFR Overexpressing Cells

LPEI-LPEI-l-[N₃:DBCO]-PEG₃₆-hEGF:hIL-2 mRNA polyplexes were formulated in HBS at N/P ratios of 3, 6 and 12. Mouse cancer cell lines with differential expression of human EGFR (Renca: no human EGFR expression; RencaEGFR M1 H: high human EGFR expression) were treated with the polyplexes. Release of IL-2 into the medium was examined 48 hours after the transfection by hIL-2 ELISA.

In detail, 15,000 cells of mouse carcinoma RencaEGFR M1 H cell line (high expression of human EGFR) and 10,000 cells of Renca (parental, human EGFR negative) were seeded into 96 well plates in triplicates and grown overnight. Human IL-2 mRNA (Trilink Biotechnologies, WOTL83314 comprising SEQ ID NO:17 (mRNA hIL-2 ORF), in 1 mM Sodium Citrate, pH 6.4) was formulated with LPEI-l-[N₃:DBCO]-PEG₃₆-hEGF in HBS (Hepes Buffered Saline, 20 mM HEPES, 150 mM NaCl, pH 7.3). The mRNA was first diluted with HBS to 0.04 mg/ml for all N/P ratios. LPEI-l-[N₃:DBCO]-PEG₃₆-hEGF was diluted with HBS to 0.0156 mg/ml (N/P 3); 0.0312 mg/ml (N/P 6) and 0.0624 mg/ml (N/P 12). The diluted LPEI-l-[N₃:DBCO]-PEG₃₆-hEGF was added to the diluted mRNA and mixed by pipetting and was incubated for 30 minutes at room temperature to form polyplex. The final concentration of LPEI-l-[N₃:DBCO]-PEG₃₆-hEGF and mRNA in the polyplexes were as follows:

| N/P ratio | 3 | 6 | 12 |
|---|---|---|---|
| Conc of mRNA (mg/ml) | 0.02 | 0.02 | 0.02 |
| Conc of LPEI-LPEI-l-[N₃:DBCO]- | 0.0078 | 0.0156 | 0.0312 |
| PEG₃₆-hEGF (mg/ml) | | | |

The mRNA was first diluted with HBS to 0.04 mg/ml for all N/P ratios. LPEI-l-[N₃:DBCO]-PEG₃₆-hEGF was diluted with HBS to 0.0156 mg/ml (N/P 3); 0.0312 mg/ml (N/P 6) and 0.0624 mg/ml (N/P 12). The diluted LPEI-l-[N₃:DBCO]-PEG₃₆-hEGF was added to the diluted mRNA and mixed by pipetting and was incubated for 30 minutes at room temperature to form polyplex. The polyplexes were serially diluted and then added to the cells (using 10× dilution) to obtain the indicated final concentrations of the mRNA (0.25, 0.5 and 1 μg/ml). The medium was collected 48 hours after the transfection and frozen at −20° C. and after thawing was subjected to human IL-2 ELISA (Peprotech, Cat #900-T12). Signal was detected using a Microplate Reader Synergy H (Biotek).

Cell survival assay: Cell viability was measured by a colorimetric Methylene Blue assay. Briefly, the cells were fixed with 2.5% Glutaraldehyde in PBS (pH 7.4), washed with double distilled water, and stained with a 1% (wt/vol) solution of methylene blue in borate buffer for 1 hour. The stain was extracted with 0.1 M HCl and the optical density of the stain solution was read at 630 nm in a microplate reader (Synergy H1, Biotek).

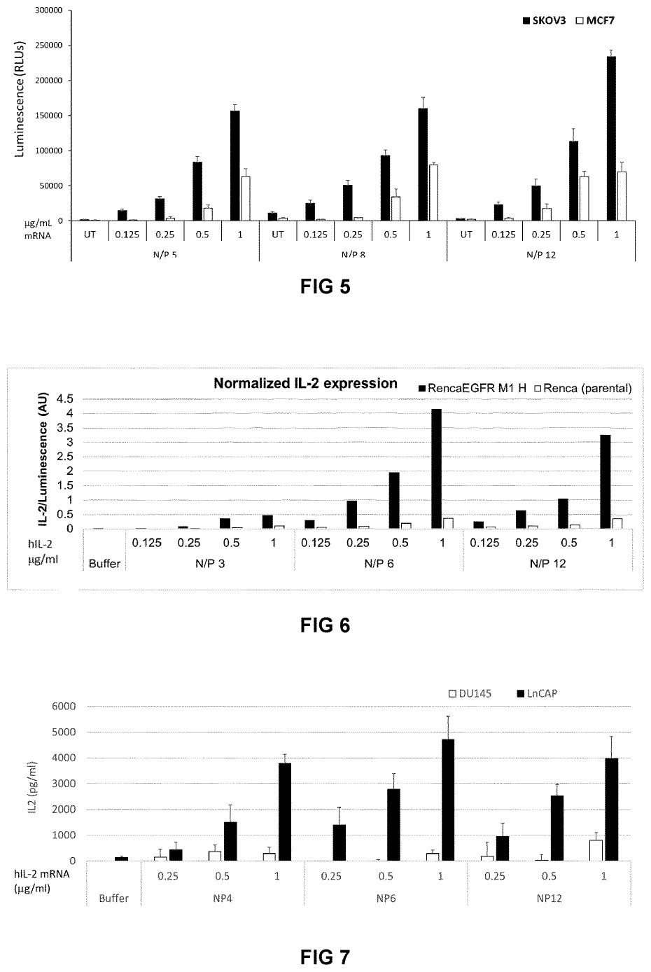

Selective delivery of LPEI-l-[N₃:DBCO]-PEG₃₆-hEGF:hIL-2 mRNA resulted in high expression and secretion of human IL-2 protein (SEQ ID NO:22) by RencaEGFR M1 H cells in a dose dependent manner, especially at N/P ratios of 6 and 12 (FIG. 6). In contrast, much lower expression and secretion of IL-2 was obtained in the medium of Renca (parental) cells.

These results demonstrate the selectivity of delivery of hTL-2 mRNA to cancer cells with high expression of EGFR and efficient IL-2 protein translation and secretion.

### Example 29

Selective Delivery of Human IL-2 mRNA Using the Inventive Polyplexes Results in High Expression of Human IL-2 in PSMA Overexpressing Cells

LPEI-l-[N₃:DBCO]-PEG₃₆-DUPA:hIL-2 mRNA polyplexes were formulated in HBS at N/P ratios of 4, 6 and 12. Prostate cancer cell lines with differential expression of PSMA (DU145: low PSMA expression; LNCaP: high PSMA expression) were treated with the polyplexes. Release of IL-2 into the medium was examined 24 hours after the transfection by IL-2 ELISA.

In detail, 15,000 cells of human prostate cancer cell lines LNCaP (high expression of PSMA) and DU145 (low expression of PSMA) were seeded into 96 well plates in triplicates and grown overnight. Human IL-2 mRNA (Trilink Biotechnologies, WOTL83314 comprising SEQ ID NO:17 (mRNA hIL-2 ORF), 0.958 mg/mL in 1 mM Sodium Citrate, pH 6.4) was formulated with LPEI-l-[N₃:DBCO]-PEG₃₆-DUPA in HBS (Hepes Buffered Saline, 20 mM HEPES, 150 mM NaCl, pH 7.3). The mRNA was first diluted with HBS to 0.04 mg/ml for all N/P ratios. LPEI-l-[N₃:DBCO]-PEG₃₆-DUPA was diluted with HBS to 0.0208 (N/P 4); 0.0312 (N/P 6) and 0.0624 (N/P 12). The diluted LPEI-l-[N₃:DBCO]-PEG₃₆-DUPA was added to the diluted mRNA and mixed by pipetting. Polyplex were formed for 30 minutes at room temperature. The final mRNA and LPEI-l-[N₃:DBCO]PEG₃₆-DUPA concentrations in the polyplexes are presented below:

| N/P ratio | 4 | 6 | 12 |
|---|---|---|---|
| Conc of mRNA (mg/ml) | 0.02 | 0.02 | 0.02 |
| Conc of LPEI-l-[N₃:DBCO]- | 0.0104 | 0.0156 | 0.0312 |
| PEG₃₆-DUPA (mg/ml) | | | |

The polyplexes were serially diluted and then added to the cells (using 10× dilution) to obtain the indicated final concentrations of the mRNA (0.25, 0.5 and 1 μg/ml). The medium was collected 24 hours after the transfection, frozen at −20° C. and after thawing was subjected to human IL-2 ELISA (Peprotech, Cat #900-T12). Signal was detected using a Microplate Reader Synergy H (Biotek).

Selective delivery of LPEI-l-[N₃:DBCO]-PEG₃₆-DUPA:hIL-2 mRNA resulted in high expression of human IL-2 protein (SEQ ID NO:22) by LNCaP cells at all N/P ratios (FIG. 7). In contrast much lower expression of IL-2 was obtained in the medium of DU145 cells.

These results demonstrate the selectivity of delivery of IL-2 mRNA to cancer cells with high expression of PSMA and the efficient IL-2 protein translation and secretion.

### Example 30

Selective Delivery of Human IFNβ mRNA Using the Inventive Polyplexes Results in High Expression of Human IFNβ in PSMA Overexpressing Cells

LPEI-l-[N₃:DBCO]PEG₃₆-DUPA:hIFNβ mRNA polyplexes were formulated in HBS at N/P ratios of 4, 6 and 12. Prostate cancer cell lines with differential expression of PSMA (DU145: low PSMA expression; LNCaP: high PSMA expression) were treated with the polyplexes. Release of IFNβ into the medium was examined 24 hours after the transfection by IFNβ ELISA.

In detail, 15,000 cells of human prostate cancer cell lines LNCaP (high expression of PSMA) and DU145 (low expression of PSMA) were seeded into 96 well plates in triplicates and grown overnight. Human IFNβ mRNA (Tebubio, TTAP-122022 comprising SEQ ID NO:18 (mRNA hIFNβ ORF) in RNAse/DNAse free water was formulated with LPEI-1-[N₃:DBCO]PEG₃₆-DUPA in HBS (Hepes Buffered Saline, 20 mM HEPES, 150 mM NaCl, pH 7.3). The mRNA was first diluted with HBS to 0.04 mg/ml for all N/P ratios. LPEI-1-[N₃:DBCO]PEG₃₆-DUPA was diluted with HBS to 0.0208 (N/P 4); 0.0312 (N/P 6) and 0.0624 (N/P 12). The diluted LPEI-l-[N₃:DBCO]PEG₃₆-DUPA was added to the diluted mRNA and mixed by pipetting. Polyplex were formed for 30 minutes at room temperature. The final mRNA and LPEI-l-[N₃:DBCO]PEG₃₆-DUPA concentrations in the polyplexes are presented below:

| N/P ratio | 4 | 6 | 12 |
|---|---|---|---|
| mRNA (mg/ml) | 0.02 | 0.02 | 0.02 |
| LPEI-l-[N₃:DBCO]PEG₃₆-DUPA (mg/ml) | 0.0104 | 0.0156 | 0.0312 |

The polyplexes were serially diluted and were added to the cells (10× dilution) to obtain the indicated final concentrations of the mRNA (0.25, 0.5 and 1.0 μg/ml). The medium was collected 24 hours after the transfection and frozen at −80° C. and after thawing was subjected to human IFNβ ELISA (InvivoGen, Catalog code: luex-hifnbv2). Signal was detected using a Microplate Reader Synergy H (Biotek).

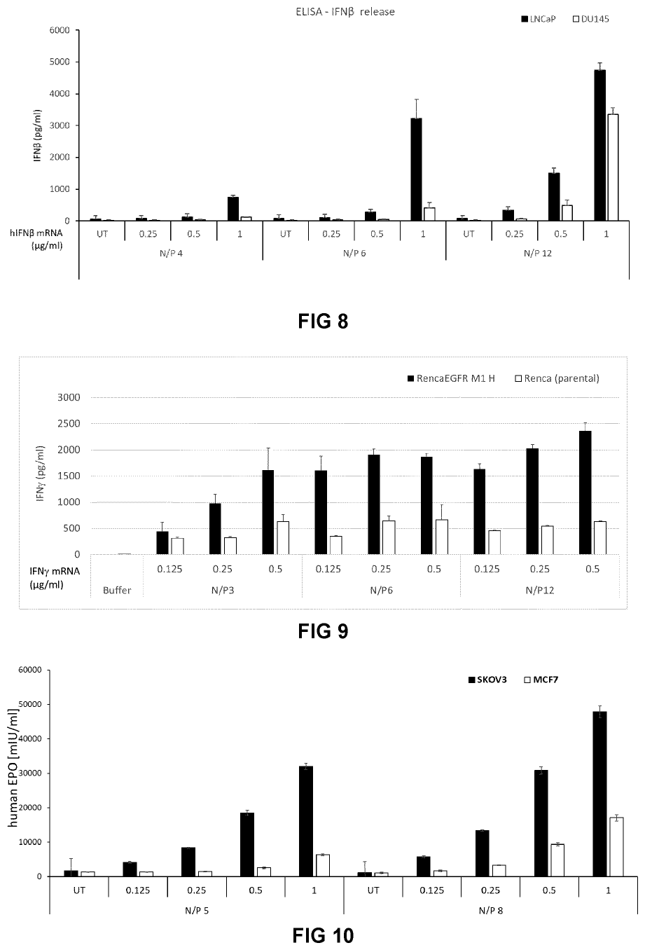

Selective delivery of LPEI-l-[N₃:DBCO]PEG₃₆-DUPA:hIFNβ mRNA resulted in high expression of human IFNβ protein (SEQ ID NO:23) by LNCaP cells at all N/P ratios at 1.0 μg/ml concentration (FIG. 8). In contrast, much lower expression of IFNβ was obtained in the medium of DU145 cells except the N/P ratio of 12 at the highest concentration (1.0 μg/ml) where higher release of IFNβ can be observed.

These results demonstrate the selective delivery of IFNβ mRNA to cancer cells with high expression of PSMA using LPEI-l-[N₃:DBCO]PEG₃₆-DUPA and efficient protein translation and secretion.

FIG. 8 depicts the levels of secreted human IFNβ from two cell lines with differential PSMA expression: PSMA high expressing LNCaP cells, and PSMA low expressing DU145 cells following transfection with PSMA targeting polyplexes containing hIFNβ mRNA (SEQ ID NO:18 (mRNA hIFNβ ORF). Selective expression of human IFNβ from PSMA high expressing cells is demonstrated.

### Example 31

Selective Delivery of Human IFNγ mRNA Using the Inventive Polyplexes Results in High Expression of Human IFNγ in EGFR High Expressing Cells

LPEI-l-[N₃:DBCO]-PEG₃₆-hEGF:hIFNγ mRNA polyplexes were formulated in HBS at N/P ratios of 3, 6 and 12. Cancer cell lines with differential expression of human EGFR receptor (Renca (parental): no human EGFR expression; RencaEGFR M1 H: high human EGFR expression) were treated with the polyplexes. Release of IFNγ into the medium was examined 24 hours after the transfection by hIFNγ ELISA.

In detail, 15,000 RencaEGFR M1 H (high expression of human EGFR) and 10,000 Renca (parental, no expression of human EGFR) were seeded into 96 well plates in triplicates and grown overnight. hIFNγ mRNA (Trilink Biotechnologies, WOTL87247 comprising SEQ ID NO:19 (mRNA hIFNγ ORF), in RNase/DNase free water) was formulated with LPEI-l-[N₃:DBCO]-PEG₃₆-hEGF in HBS (Hepes Buffered Saline, 20 mM HEPES, 150 mM NaCl, pH 7.3). The mRNA was first diluted with HBS to 0.04 mg/ml for all N/P ratios. LPEI-l-[N₃:DBCO]-PEG₃₆-hEGF was diluted with HBS to 0.0156 mg/ml (N/P 3); 0.0312 mg/ml (N/P 6) and 0.0624 mg/ml (N/P 12). The diluted LPEI-l-[N₃:DBCO]-PEG₃₆-hEGF was added to the diluted mRNA and mixed by pipetting and incubated for 30 minutes at room temperature to form polyplexes. The final concentrations of LPEI-l-[N₃:DBCO]-PEG₃₆-hEGF and mRNA in the polyplexes were as follows:

| N/P ratio | 3 | 6 | 12 |
|---|---|---|---|
| Conc of mRNA (mg/ml) | 0.02 | 0.02 | 0.02 |
| Conc of LPEI-l-[N₃:DBCO]- | 0.0078 | 0.0156 | 0.0312 |
| PEG₃₆-hEGF (mg/ml) | | | |

The polyplexes were diluted using serial dilutions and then added to the cells (10× dilution) to obtain the indicated final concentrations of the mRNA (0.125, 0.25 and 0.5 μg/ml). The medium was collected 24 hours after the transfection and frozen at −20° C. Medium was defrosted, diluted 3-fold with ELISA diluent and subjected to hIFNγ ELISA and conducted according to manufacturer's instructions (BD Biosciences, cat #BD555142). Signal was detected using a Microplate Reader Synergy H (Biotek). Quantification of the secreted hIFNγ protein was adjusted according to the dilution factor.

Selective delivery of LPEI-l-[N₃:DBCO]-PEG₃₆-hEGF: hIFNγ mRNA resulted in high expression and secretion of IFNγ protein (SEQ ID NO:24) by RencaEGFR M1 H (FIG. 9) at all N/P ratios. In contrast, much lower expression of IFNγ was obtained in the medium of Renca (parental) cells.

These results demonstrate the selectivity of delivery of hIFNγ mRNA to cancer cells with high expression of EGFR and efficient protein translation and secretion.

### Example 32

Selective Delivery of Human EPO mRNA Using the Inventive Polyplexes Results in High Expression and Secretion of Human EPO (hEPO) In High Folate Receptor-Expressing Cells

LPEI-1-[N3:DBCO]-PEG24-Folate:hEPO mRNA polyplexes were formulated in HBS at N/P ratios of 5 or 8. Cancer cell lines with differential expression of folate receptor (SKOV3: high folate receptor expression; MCF7: low folate receptor expression) were treated with the polyplexes. Release of EPO into the medium was examined 24 hours after the transfection by hEPO ELISA (ThermoFisher Scientific, BMS2035-2).

In detail, SKOV3 cells (15,000 cells/well) and MCF7 cells (15,000 cells/well) were seeded into 96 well plates in quadruplicates and grown overnight. hEPO mRNA (Trilink Biotechnologies, L-7209 comprising SEQ ID NO:20 (mRNA EPO ORF) was formulated with LPEI-1-[N3:DBCO]-PEG24-Folate in HBS (Hepes Buffered Saline, 20 mM HEPES, 150 mM NaCl, pH 7.3) at the indicated N/P ratios.

The mRNA was first diluted with HBS to 0.04 mg/ml for all N/P ratios. LPEI-l-[N3:DBCO]-PEG24-Folate was diluted with HBS to 0.026 mg/ml (N/P 5) and 0.042 mg/ml (N/P 8). The diluted LPEI-1-[N3:DBCO]-PEG24-Folate was added to the diluted mRNA and mixed by pipetting and incubated for 30 minutes at room temperature to form polyplexes. The final concentrations of mRNA and LPEI-l-[N3:DBCO]-PEG24-Folate in the polyplexes are described below:

| N/P ratio | 5 | 8 |
|---|---|---|
| Conc of mRNA (mg/ml) | 0.02 | 0.02 |
| Conc of LPEI-l-[N₃:DBCO]-PEG₂₄-Folate (mg/ml) | 0.013 | 0.021 |

The polyplexes were then serially diluted and added to the cells to obtain the indicated final concentrations of the mRNA (0.125, 0.25, 0.5 and 1.0 μg/mL). The medium was collected 24 hours after the transfection and frozen at −20° C. After thawing the medium was diluted 1:200 and was then subjected to hEPO ELISA (ThermoFisher Scientific, BMS2035-2). Signal was detected using a Berthold Technologies, Mithras² μLB 943 Multimode Reader.

Selective delivery of LPEI-l-[N₃:DBCO]-PEG₂₄-Folate:hEPO mRNA resulted in high expression and secretion of hEPO by high folate receptor (FR) expressing SKOV3 cells at all the tested N/P ratios in a dose-dependent manner (FIG. 10). In contrast, substantially lower expression of hEPO was detected in the medium of MCF7 cells.

These results demonstrate the selectivity of delivery of hEPO mRNA to cancer cells with high expression of folate receptor and the efficient protein translation and secretion.

### Example 33

Selective Delivery of Diphtheria Toxin (DT) Catalytic Domain A (DT-A) mRNA Using the Inventive Polyplexes Results in Inhibition of Protein Biosynthesis in PSMA High Expressing Cells

DT-A inhibits the enzymatic ADP-ribosylation of elongation factor 2, thereby blocking the translational machinery of target cells. The antibiotic puromycin binds to newly synthesized polypeptide chains which can then be detected by Western blot analysis using an anti-puromycin antibody. Reduction in detection indicates inhibition of protein biosynthesis. The selective inhibition of protein biosynthesis mediated by targeted delivery of mRNA encoding DT-A and the consequent DT-A protein expression in high PSMA expressing cells was examined by western blot analysis using anti-Puromycin antibody.

LPEI-l-[N₃:DBCO]PEG₃₆-DUPA:DT-A mRNA polyplexes were formulated in 5% glucose at N/P ratios of 4, 6 and 12. Prostate cancer cell lines with differential expression of PSMA (DU145: low PSMA expression; LNCaP: high PSMA expression) were treated with the polyplexes for 24 hours followed by puromycin treatment for 15 minutes.

In detail, human prostate LNCaP (high expression of PSMA) and DU145 (low expression of PSMA) cancer cells were seeded into 6 well plates (400,000 cells/well) and grown overnight. DT-A mRNA (Tebubio, TTAP-012023 comprising SEQ ID NO:21 (mRNA DT-A ORF) was formulated with LPEI[N3:DBCO]PEG36-DUPA in 5% glucose at 0.1 mg/ml at the indicated N/P ratio of 4 with mRNA concentration of 0.1 mg/ml and LPEI-l-[N₃:DBCO]PEG₃₆-DUPA concentration of 0.052 mg/ml.

The mRNA was first diluted with 5% glucose to 0.2 mg/ml. LPEI-l-[N₃:DBCO]PEG₃₆-DUPA was diluted with 5% glucose 0.104 mg/ml (N/P ratio 4). The diluted LPEI-l-[N₃:DBCO]PEG₃₆-DUPA was added to the diluted mRNA (equal volumes of each) and mixed by pipetting. Polyplexes were allowed to form for 30 minutes at room temperature. The polyplexes were serially diluted and then added to the cells (using 10×dilution) to obtain the indicated final concentrations of mRNA (0.25, 0.5 and 1.0 μg/ml). After 24 hours of treatment, cells were treated with 5 μg/ml puromycin (Med Chem Express HY-B1743A) for 15 minutes, then harvested and the protein lysates were prepared. 30 μg of each protein lysate were run on 4-20% Mini-PROTEAN® TGX™ Precast Protein Gels (BioRad) before being transferred to 0.2 μm PVDF membranes (BioRad). Protein biosynthesis inhibition was detected by an anti-Puromycin antibody (Sigma/Merck, MABE343) and GAPDH (Cell signaling 2118) was used as a loading control.

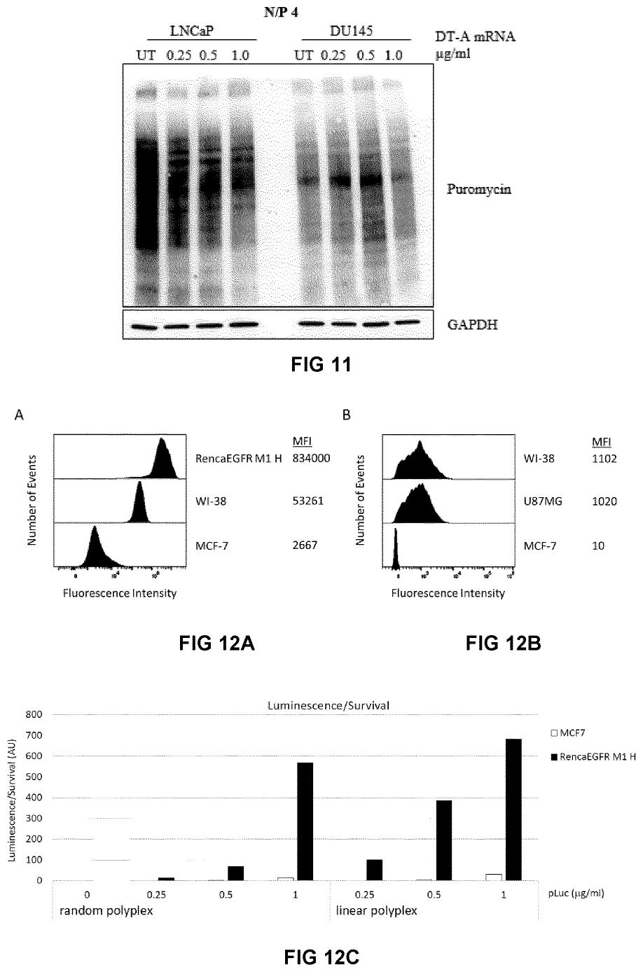

Selective delivery of LPEI-l-[N₃:DBCO]PEG₃₆-DUPA:DT-A mRNA at N/P ratio of 4 resulted in dose dependent inhibition of protein biosynthesis in LNCaP cells (FIG. 11). In contrast, no inhibition of protein biosynthesis was observed in DU145 cells. These results demonstrate the selectivity of delivery of DT-A mRNA to cancer cells with high expression of PSMA and efficient expression of functional DT-A protein which resulted in a dose dependent inhibition of protein synthesis (SEQ ID NO:25).

### Example 34

Selective Delivery of SARS-CoV-2 Spike mRNA By the Inventive Polyplexes Results in High Expression of the Spike Protein in PSMA-High Expressing Cells

Inventive polyplexes targeting PSMA and containing mRNA encoding the SARS-Cov-2 S protein are formulated in HBS (Hepes Buffered Saline, 20 mM HEPES, 150 mM NaCl, pH 7.3) at N/P ratios of 4, 6, or 12.

Cancer cell lines with differential expression of PSMA (LNCaP: high PSMA expression; DU145: no PSMA expression) are transfected with the polyplexes. Cells are harvested 24 hours after transfection and protein expression is determined by western-blot analysis.

In detail, 400,000 cells of human prostate cancer cell lines LNCaP (high expression of PSMA) and DU145 (low expression of PSMA) are seeded into 6 well plates and grown overnight. SARS-CoV-2 S mRNA (Trilink Biotechnologies) is formulated with LPEI-l-[N₃:DBCO]-PEG₃₆-DUPA in HBS (Hepes Buffered Saline, 20 mM HEPES, 150 mM NaCl, pH 7.3) at 0.02 mg/ml at the indicated N/P ratios.

| N/P ratio | 4 | 6 | 12 |
|---|---|---|---|
| Conc of mRNA (mg/ml) | 0.02 | 0.02 | 0.02 |
| Conc of LPEI-l-[N₃:DBCO]- | 0.0104 | 0.0156 | 0.0312 |
| PEG₃₆-DUPA (mg/ml) | | | |

The mRNA is first diluted with HBS to 0.04 mg/ml for all N/P ratios. LPEI-l-[N₃:DBCO]PEG₃₆-DUPA is diluted with HBS to 0.0208 mg/ml (N/P 4); 0.0312 mg/ml (N/P 6) and 0.0624 mg/ml (N/P 12). The diluted LPEI-l-[N₃:DBCO]PEG₃₆-DUPA is added to the diluted mRNA and mixed by pipetting and incubated for 30 minutes at room temperature to form polyplexes. The polyplexes is serially diluted and added to the cells (using 10× dilution) to obtain the indicated final concentrations (0.25, 0.5 and 1.0 μg/ml) of the mRNA. Cells are lysed after 24 hours of treatment and lysates are prepared. Protein lysate are run on 4-20% Mini-PROTEAN® TGX™ Precast Protein Gels (BioRad) before being transferred onto 0.2 μm PVDF membranes (BioRad). S protein expression is detected by an α-Spike antibody (Sino Biological, Cat #40591-MM42) and f-actin (Sigma Aldrich, Cat #A5441) is used as a loading control.

### Example 35

### Selective Delivery of Various Plasmids Encoding Luciferase Using The Inventive Polyplexes Results in Selective Expression of Luciferase in EGFR Overexpressing Cells

LPEI-l-[N₃:DBCO]-PEG₃₆-hEGF:pGreenFire1-CMV polyplexes and polyplexes of plasmidpGreenFire1-CMV with branched, random triconjugate comprising LPEI fragment, PEG fragment and the targeting fragment hEGF as analogously described in WO 2015/173824 were formulated in HBS at N/P ratio of 6. Cells with differential expression of human EGFR receptor (RencaEGFR M1 H: high EGFR expression; WI-38 and U87MG (medium EGFR), MCF7 and HUVEC (low EGFR) were treated with the two polyplexes. Luminescence was measured 30 hours after the transfection and detected using Luminoskan Ascent Microplate Luminometer (Thermo Scientific). Luminescence was normalized to cell survival.

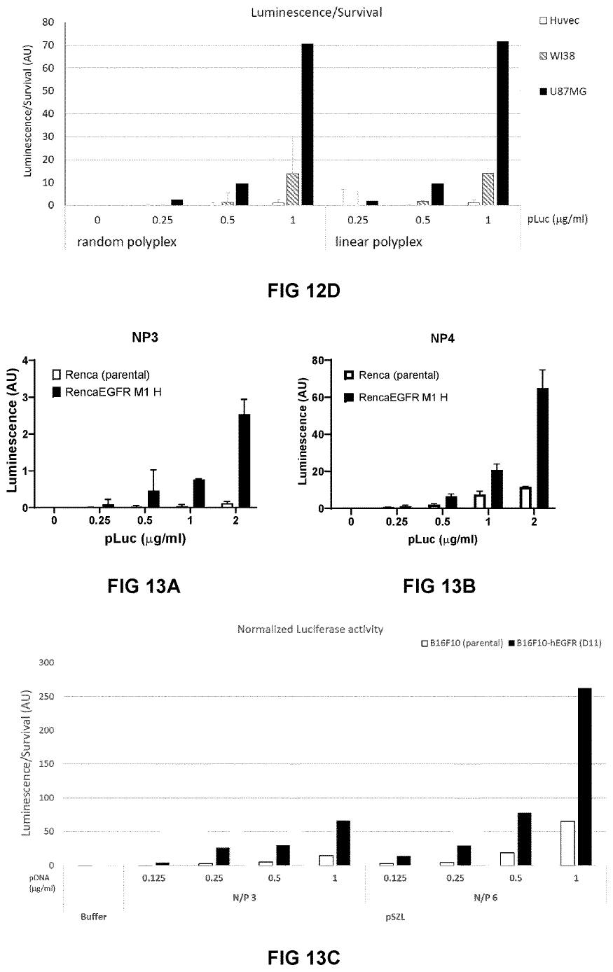

In detail, first differential human EGFR cell surface expression is demonstrated. RencaEGFR M1 H cells show high EGFR expression; U87MG and WI-38 cells, moderate EGFR expression; and MCF-7 cells show low EGFR expression (FIG. 12 A and FIG. 12B). Hereto, 106 cells from each line were washed with the cold FACS buffer (Invitrogen 00-4222-26) and stained with PE-anti hEGFR antibody (Biolegend, Cat #352904) in 100 μL FACS buffer for 30 minutes on ice in the dark. Cells were washed, resuspended in 200 μL FACS buffer and analyzed for human EGFR expression using either BD FACS Aria (FIG. 12A) or Beckman Coulter CytoFLEX S (FIG. 12B).

Further, mouse renal carcinoma RencaEGFR M1 H cells, which express high levels of human EGFR were seeded into 96-well plates at 15,000 cells per well. Human MCF7 cells, which express low levels of EGFR, were seeded at 10,000 cells per well to adjust for similar cell numbers on the day of treatment as they have faster proliferation rate. After overnight incubation the cells were transfected with the described two polyplexes.

The two polyplexes were formulated in HBS (HEPES Buffered Saline, 20 mM HEPES, 150 mM NaCl, pH 7.3) as follows: The plasmid was diluted with HBS to 0.04 mg/ml, the respective linear and random triconjugates were diluted with HBS to 0.0312 mg/ml. The diluted respective linear and random triconjugates were each added to the diluted plasmid in equal volumes and mixed by pipetting (Plasmid concentration=0.02 mg/ml; triconjugate concentration=0.0156 mg/ml; N/P=6). Polyplexes were allowed to form for 30 minutes at room temperature. The polyplexes were serially diluted and then added to the cells (further 10-fold dilution) to obtain the indicated final concentrations of the plasmid (0.25, 0.5 and 1.0 μg/ml). All conditions were tested on triplicate wells. Luminescence was measured 30 hours after transfection with ONE-Glo™ EX Luciferase Assay System (Promega, Cat #E8130) using Luminoskan Ascent Microplate Luminometer (Thermo Labsystems).

Selective delivery of LPEI-l-[N₃:DBCO]-PEG₃₆-hEGF:pGreenFire1-CMV and of the random polyplex resulted in robust expression of Luciferase in RencaEGFR M H1 cells. The strength of the luminescence signal was dose dependent. Higher expression and activity were demonstrated following transfection with LPEI-l-[N₃:DBCO]-PEG₃₆-hEGF:pGreenFire1-CMV as compared to the polyplex of pGreenFire1-CMV with the random triconjugate. Low to no luminescence signal was detected in the low EGFR-expressing MCF7 cells (FIG. 12C) following treatment with either of the polyplexes.

Plasmid import occurs passively during mitosis, due to the breakdown of the nuclear membrane. Thus, rapidly dividing cancer cells should be transfected more efficiently than non-dividing or slowly dividing non-cancer cells. Indeed, moderate levels of luciferase were detected in the rapidly proliferating U87MG cancer cells, which express moderate levels of EGFR, whereas substantially lower levels of luminescence were detected in the non-cancerous WI-38 cells, which express similar moderate levels of EGFR but proliferate slowly. No luminescence was detected in the non-cancerous, slowly proliferating HUVEC cells with low EGFR expression (FIG. 12D).

The results demonstrate that both EGFR-targeting polyplexes containing luciferase-encoding plasmid can differentiate between high (RencaEGFR M H1), medium (U87MG) and low (MCF7) EGFR-expressing cancer cells and non-cancer cells (WI-38, HUVEC) with low proliferation rates and medium to low EGFR expression, while higher expression and activity were demonstrated following transfection with LPEI-l-[N₃:DBCO]-PEG₃₆-hEGF:pGreenFire1-CMV as compared to the polyplex of pGreenFire1-CMV with the random triconjugate.

In addition, polyplexes comprising Fluc pDNA (pSZL) and LPEI-l-[N₃:DBCO]-PEG₃₆-hEGF (i.e., Compounds 70a and 70b) were generated by complexing the pSZL in HEPES-buffered saline (HBS: 20 mM HEPES, 150 mM NaCl, pH 7.2) at 0.02 mg/ml with the triconjugate LPEI-l-[N₃:DBCO]-PEG₃₆-hEGF at N/P ratio of 3 and 6 (where N=nitrogen from LPEI and P=phosphate of pSZL). To allow complete formation of the polyplex particles, i.e., LPEI-l-[N₃:DBCO]PEG₃₆-hEGF:[pSZL], the samples were incubated for 30 min at room temperature.

B16F10-hEGFR (a derivate of the mouse melanoma cell line, B16F10, was engineered to overexpress human EGFR) and B16F10 (parental) cells were cultured in DMEM medium supplemented with 10% fetal bovine serum (FBS), 10⁴ U/L penicillin, 10 mg/L streptomycin at 37° C. in 5% CO₂. 25 μg/ml of Zeocin was added to the medium of B16F10-hEGFR cells. 500 cells were seeded in triplicates at 90 μl into 96 well white plates (Greiner). Cells were transfected with 0.125-1.0 mg/ml LPEI-l-[N₃:DBCO]PEG₃₆-hEGF:[pSZL]. The medium was exchanged 48 hrs after the transfection.

Luciferase activity was measured with OneGloX assay (Promega) 6 days after the treatment. Luminescence measurements were performed using a Luminoskan Ascent Microplate Luminometer (Thermo Scientific).

Cell survival assay: Cell viability was measured by a colorimetric Methylene Blue assay. Briefly, the cells were fixed with 2.5% Glutaraldehyde in PBS (pH 7.4), washed with double distilled water, and then stained with a 1% (wt/vol) solution of methylene blue in borate buffer for 1 hr. The stain was extracted with 0.1 M HCl and the optical density of the stain solution was read at 630 nm in a microplate reader (Synergy H1, Biotek).

FIG. 13C depicts relative luminescence (AU) normalized to survival in B16F10-hEGFR and B16F10 parentals cells treated with LPEI-l-[N₃:DBCO]PEG₃₆-hEGF:[pSZL]6 days after treatment at an N/P ratio of 3 and 6.

This data demonstrate selective pDNA delivery to B16F10-hEGFR cells utilizing LPEI-l-[N₃:DBCO]PEG₃₆-hEGF:[pSZL] at N/P ratios of 3 and 6. Remarkably, significant luciferase expression was obtained even with very low number of cells (500) and was maintained for at least 6 days.

### Example 36

Selective Delivery of a Plasmid that Encodes Luciferase Using The Inventive Polyplexes at Low N/P Ratios Results in Luminescence in High EGFR Expressing Cells

LPEI-l-[N₃:DBCO]-PEG₃₆-hEGF:pGreenFire1-CMV polyplexes were formulated in HBS at N/P ratios of 3 and 4. Cells with differential expression of human EGFR (RencaEGFR M1 H (high EGFR expression); Renca parental: EGFR negative)) were treated with the polyplexes. Luminescence was measured 30 hours after the transfection and detected using a Luminoskan Ascent Microplate Luminometer (Thermo Scientific).

In detail, mouse renal carcinoma RencaEGFR M1 H cells (high expression of human EGFR; 15,000 cells/well) and Renca (parental, human EGFR negative; 10,000 cells/well) were seeded into 96-well plates and grown overnight. Triplicate wells were prepared for each condition. pGreenFire1-CMV was formulated with LPEI-l-[N₃:DBCO]-PEG₃₆-hEGF in HBS (HEPES Buffered Saline, 20 mM HEPES, 150 mM NaCl, pH 7.3).

The plasmid was first diluted with HBS to 0.04 mg/ml for all N/P ratios. LPEI-l-[N₃:DBCO]-PEG₃₆-hEGF was diluted with HBS to 0.0156 mg/ml (N/P 3); 0.0208 mg/ml (N/P 4). Equal volumes of the diluted LPEI-l-[N₃:DBCO]-PEG₃₆-hEGF were added to the diluted plasmid and mixed by pipetting. Polyplexes were formed for 30 minutes at room temperature.

The final concentrations of LPEI-l-[N₃:DBCO]-PEG₃₆-hEGF and plasmid in the polyplexes are described below:

| N/P ratio | 3 | 4 |
|---|---|---|
| Conc of plasmid (mg/ml) | 0.02 | 0.02 |
| Conc of LPEI-l-[N₃:DBCO]-PEG₃₆-hEGF (mg/ml) | 0.0078 | 0.0104 |

The polyplexes were serially diluted and then added to the cells to obtain the indicated final concentrations of the plasmid (0.25, 0.5, 1 and 2.0 μg/ml). Luciferase activity was measured 30 hours after the transfection with ONE-Glo™ EX Luciferase Assay System (Promega, Cat #E8130) using Luminoskan Ascent Microplate Luminometer (Thermo Labsystems).

Selective delivery of the LPEI-l-[N₃:DBCO]-PEG₃₆-hEGF:pGreenFire1-CMV polyplexes at low N/P ratios of 3 and 4, resulted in higher luminescence in RencaEGFR M H1 (high EGFR) cells than in parental Renca cells (no human EGFR). The detected increase in luminescence signal was dose dependent (FIG. 13A and FIG. 13B).

These results demonstrate the selective delivery of LPEI-l-[N₃:DBCO]-PEG₃₆-hEGF:pGreenFire1-CMV polyplexes at low N/P ratios to cancer cells with high EGFR expression, and the consequent efficient luciferase protein translation and activity.

### Example 37

### Selective Delivery of Plasmid DNA Encoding Luciferase Utilizing the Inventive Polyplexes Results in High Expression of Luciferase in PSMA Overexpressing Cells

LPEI-l-[N₃:DBCO]-PEG₃₆-DUPA:pGreenFire-CMV polyplexes were formulated in HBS at N/P ratios of 4 and 6. Prostate cancer cell lines with differential expression of PSMA (DU145: low PSMA expression; LNCaP: high PSMA expression) were treated with the polyplexes. Luminescence was measured 24 hours after the transfection.

In detail, cells were seeded into 96-well plates (15000 cells/well) and grown overnight. pLuc plasmid (pGreenFire-CMV, SBI) was formulated with LPEI-l-[N₃:DBCO]-PEG₃₆-DUPA in HBS (HEPES Buffered Saline, 20 mM HEPES, 150 mM NaCl, pH 7.3) at 0.02 mg/ml at the indicated N/P ratios, diluted and added to the cells, to obtain the indicated final concentrations of the plasmid. Triplicate samples were tested for each condition. Luminescence was measured 24 hours after the transfection with the ONE-Glo™ EX Luciferase Assay System (Promega).

Selective delivery of LPEI-l-[N₃:DBCO]-PEG₃₆-DUPA:pGreenFire-CMV resulted in high luminescence signals in LNCaP cells in a dose dependent manner at N/P 6 (FIG. 24). In contrast, much lower expression of luciferase was obtained in DU145 cells.

These results demonstrate the selective delivery of plasmid DNA encoding luciferase to cancer cells with high expression of PSMA and efficient protein expression and activity.

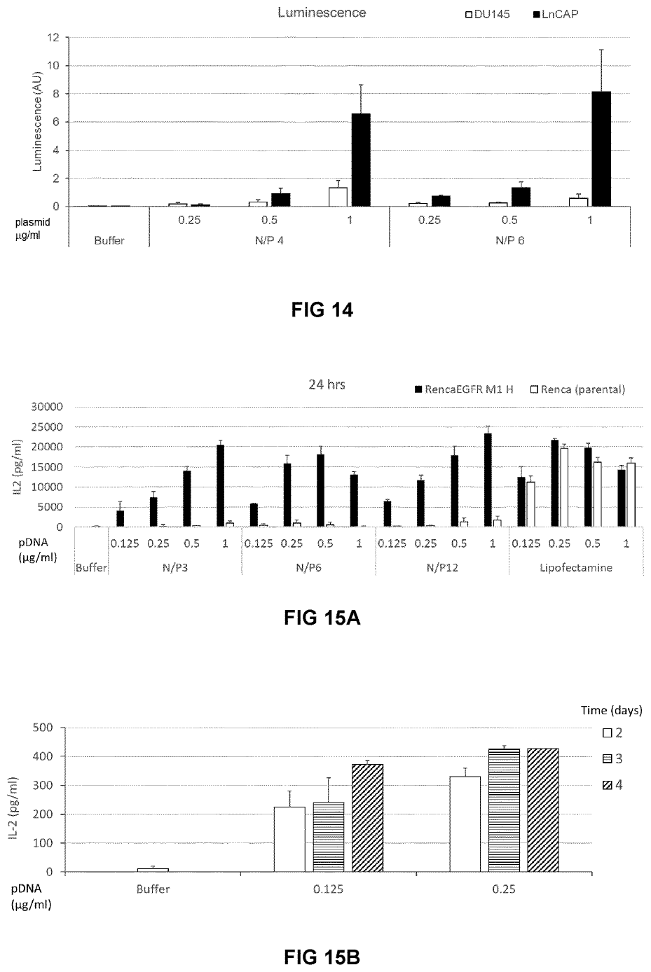

FIG. 14 depicts luminescence from human prostate cell lines with differential cell surface expression of PSMA: high-PSMA expressing LNCaP cells, and low PSMA-expressing DU145 cells. The cells were treated with PSMA-targeting polyplexes containing plasmid DNA encoding luciferase. The X axis indicates the concentration of the pGreenFire-CMV in the polyplexes (0.25, 0.5 and 1.0 μg/mL). The Y axis indicates luminescence in arbitrary units (AU). Average and standard deviation from triplicate samples are presented. Selective expression of luciferase after transfection of PSMA overexpressing cells with plasmid DNA encoding luciferase (pGreenFire-CMV) is demonstrated.

### Example 38

### Selective Delivery of Plasmid DNA Encoding Human IL2 Using the Inventive Polyplexes Results in High Expression of Human IL2 in EGFR Overexpressing Cells

LPEI-l-[N₃:DBCO]-PEG₃₆-hEGF:pUNO-hIL2 polyplexes were formulated in HBS at N/P ratios of 3, 6 and 12. Cancer cell lines with differential expression of human EGFR receptor (Renca (parental): no EGFR expression; RencaEGFR M1 H: high EGFR expression) were treated with the polyplexes. Lipofectamine/pUNO-hIL2 was used as a positive control for transfection. Release of human IL2 into the medium was examined at 24 hours after the transfection by human IL-2 ELISA.

In detail, mouse renal carcinoma cells with high (RencaEGFR M1 H) or no (Renca, parental) expression of human EGFR were seeded into 96 well plates in triplicates (15,000 cells/well for RencaEGFR M1 H and 10,000 cells/well for Renca cells) and grown overnight. pUNO-hIL2 (InvivoGen, comprising SEQ ID NO:26) was formulated with LPEI-l-[N₃:DBCO]-PEG₃₆-hEGF in HBS (HEPES Buffered Saline, 20 mM HEPES, 150 mM NaCl, pH 7.3) at 0.02 mg/ml at the indicated N/P ratios:

| N/P ratio | 3 | 6 | 12 |
|---|---|---|---|
| Conc of plasmid (mg/ml) | 0.02 | 0.02 | 0.02 |
| Conc of LPEI-l-[N₃:DBCO]- | 0.0078 | 0.0156 | 0.0312 |
| PEG₃₆-hEGF (mg/ml) | | | |

The plasmid was diluted with HBS to 0.04 mg/ml for all N/P ratios. LPEI-l-[N₃:DBCO]-PEG₃₆-hEGF was diluted with HBS to 0.0156 mg/ml (N/P 3), 0.0312 mg/ml (N/P 6) or 0.0624 mg/ml (N/P 12). The diluted LPEI-l-[N₃:DBCO]-PEG₃₆-hEGF was added to the diluted plasmid and mixed by pipetting. Polyplexes were formed for 30 minutes at room temperature. The polyplexes were serially diluted and added to the cells (with a further 10-fold dilution) to obtain the indicated final concentrations of the plasmid (0.125, 0.25, 0.5 and 1.0 μg/ml). Commercial transfection agent (Lipofectamine 3000, ThermoFisher Scientific, Cat #L3000008) was used as positive control. Lipofectamine/pUNO-hIL2 transfection mixture was prepared according to the manufacturer's instructions and added to the cells to obtain the same final concentrations of the plasmid (0.125, 0.25, 0.5 and 1.0 μg/ml). The medium was collected 24 hours after the transfection, stored at −20° C. and after thawing was subjected to human IL-2 ELISA (Peprotech, Cat #900-T12). Signal was detected using a Microplate Reader Synergy H (Biotek).

Selective delivery of LPEI-l-[N₃:DBCO]-PEG₃₆-hEGF:pUNO-hIL2 resulted in high expression and secretion of human IL2 protein (SEQ ID NO:22) up to 25 ng/ml by RencaEGFR M1 H at all N/P ratios (FIG. 15A). In contrast, significant lower expression of IL2 was obtained in the medium of Renca (parental) cells. As expected, treatment with the positive control, Lipofectamine, did not show any selectivity and resulted in similar expression and secretion of IL2 from both cell lines EGFR high expressing RencaEGFR M1 H and EGFR negative Renca (parental) cells.

These results demonstrate selective delivery of plasmid DNA encoding human IL2 to cancer cells with high expression of EGFR and efficient protein translation and secretion at all N/P ratios.

Efficient secretion of IL2 in the medium was obtained at significant lower number of RencaEGFR M1 H cells (600 cells) after transfection with lower concentrations of LPEI-l-[N₃:DBCO]-PEG₃₆-hEGF:pUNO-hIL2, 0.125 and 0.25 μg/ml. Furthermore, IL-2 secretion was maintained at least up to 4 days (FIG. 15B). Such an unexpected high expression and duration may have a significant impact on the efficiency of the inventive polyplexes in vivo (Vetter V C, Wagner E. J Control Release, 2022 346:110-135). Our results indicate that even with low percentage of the transfected cancer cells high and stable expression of an immune activator can be obtained. Strong stimulation of the immune cells by the expressed immune activator can lead to elimination of not only transfected cells but also non-transfected cancer cells regardless of the expression of EGFR (bystander effect). Yet, the activation of the immune system is expected in the vicinity of tumor only as only the cancer cells are expected to express the immune activator. Thus, both high potency and high selectivity are ensured.

### Example 39

### Selective Delivery of Plasmid DNA Encoding Human IL2 Using the Inventive Polyplexes Results in High Expression of Human IL2 in PSMA Overexpressing Cells

LPEI-l-[N₃:DBCO]-PEG₃₆-DUPA:pUNO1-hIL2 polyplexes were formulated in HBS at N/P ratios of 4, 6 and 12. Prostate cancer cell lines with differential expression of PSMA (DU145: low PSMA expression; C4-2, LNCaP: high PSMA expression (Juzeniene A et al, Cancers 2021, 13(4):779) were treated with the polyplexes. Release of human IL2 into the medium was examined 24 hours after the transfection by human IL-2 ELISA.

In detail, human prostate cancer cells that express high (LNCaP, C4-2) or low (DU145) levels of PSMA were seeded into 96-well plates in triplicates (15,000 cells/well) and grown overnight. Plasmid DNA encoding hIL2 (pUNO1-hIL2, InvivoGen, comprising SEQ ID NO:26) was formulated with LPEI-l-[N₃:DBCO]-PEG₃₆-DUPA in HBS (HEPES Buffered Saline, 20 mM HEPES, 150 mM NaCl, pH 7.3). pUNO1-hIL2 was first diluted with HBS to 0.04 mg/ml for all N/P ratios. LPEI-l-[N₃:DBCO]-PEG₃₆-DUPA was diluted with HBS to 0.0208 (N/P 4); 0.0312 (N/P 6) and 0.0624 (N/P 12). The diluted LPEI-l-[N₃:DBCO]-PEG₃₆-DUPA was added to the diluted pUNO1-hIL2 and mixed by pipetting. Polyplex were formed for 30 minutes at room temperature. The final concentrations of plasmid DNA and LPEI-1-[N₃:DBCO]-PEG₃₆-DUPA in the nanoparticles were as follows:

| N/P ratio | 4 | 6 | 12 |
|---|---|---|---|
| Conc of plasmid (mg/ml) | 0.02 | 0.02 | 0.02 |
| Conc of LPEI-l-[N₃:DBCO]- | 0.0104 | 0.0156 | 0.0312 |
| PEG₃₆-DUPA (mg/ml) | | | |

The polyplexes were serially diluted and were added to the cells to obtain the indicated final concentrations of the plasmid (0.25, 0.5 and 1.0 μg/ml).

The medium was collected 24 hours after the transfection and stored at −20° C. Medium was thawed and subjected to human IL2 ELISA (Peprotech, Cat #900-T12). Signals from ELISA were detected using a Microplate Reader Synergy H (Biotek). Survival of the cells was measured with CellTiter-Glo (Promega, Cat #G7571) using Luminoskan Ascent Microplate Luminometer (Thermo Labsystems). Normalized IL2 concentrations were obtained by dividing the average of IL2 concentrations by the average luminescence (survival).

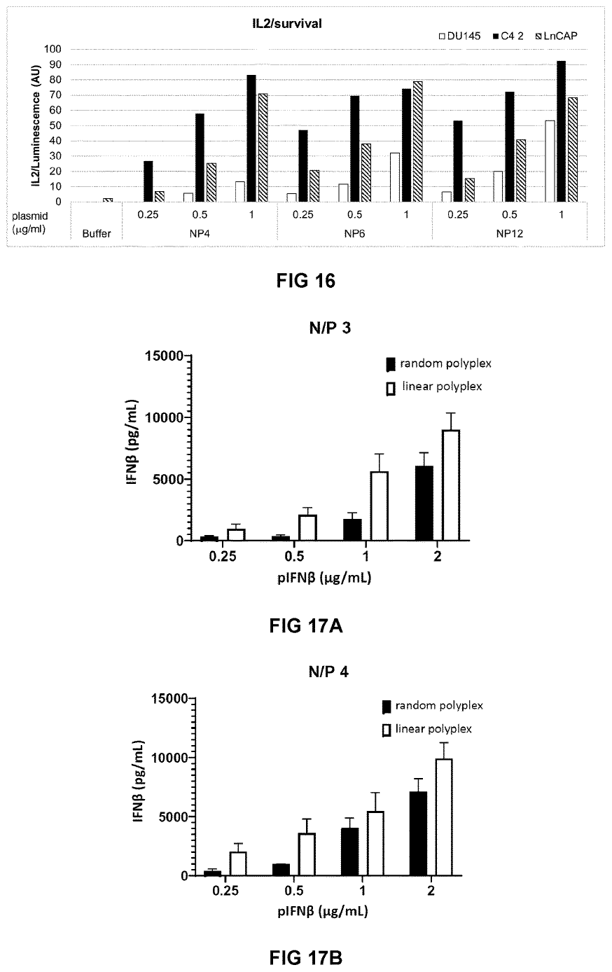

Delivery of LPEI-l-[N₃:DBCO]-PEG₃₆-DUPA:pUNO1-hIL2 resulted in high expression of human IL2 by LNCaP and C4-2 cells at all N/P ratios (FIG. 16). In contrast, much lower expression of IL2 was obtained in the medium of DU145 cells at all N/P ratios.

These results demonstrate the selective delivery of plasmid DNA encoding human IL2 protein (SEQ ID NO:22) to cancer cells with high expression of PSMA at all N/P ratios as well as efficient translation and secretion of the encoded IL-2 protein.

FIG. 16 depicts levels of secreted human IL2 normalized to cell survival, in cell lines with differential PSMA expression: high-expressing LNCaP and C4-2 cells, and low-expressing DU145 cells following transfection with PSMA-targeting polyplexes containing plasmid encoding IL2 protein. The X axis indicates the concentration of pUNO1-hIL2 plasmid DNA (0.25, 0.5 and 1.0 μg/mL) in the polyplexes. The Y axis indicates the concentration of secreted IL-2 normalized to cell survival in Arbitrary Units (AU). The selective expression/secretion of human IL2 after transfection of PSMA overexpressing cells with plasmid DNA encoding hIL-2 is demonstrated.

### Example 40

Delivery of pCMV-hIFNβ Utilizing the Inventive Polyplexes Results in High Expression of Human IFNβ in EGFR Overexpressing Cancer Cells

LPEI-l-[N₃:DBCO]-PEG₃₆-hEGF:pCMV-hIFNβ polyplexes (referred to as linear polyplexes) and polyplexes of pCMV-hIFNβ with branched, random triconjugate comprising LPEI fragment, PEG fragment and the targeting fragment hEGF as analogously described in WO 2015/173824 (referred to as random polyplexes) were formulated in HBS at N/P ratios of 3 and 4. Cancer cells with high expression of human EGFR (RencaEGFR M1 H) were treated with the polyplexes. Secretion of human IFNβ into the medium was examined-by hIFNβ ELISA 24 hours after the transfection.

In detail, 15,000 RencaEGFR M1 H cells (high expression of human EGFR) were seeded into 96-well plates in triplicates and grown overnight. pCMV-hIFNβ (Sino Biological, pCMV3-hIFNb comprising SEQ ID NO:27) was formulated with the described two triconjugates (referred to as linear and random triconjugates) in HBS (HEPES Buffered Saline, 20 mM HEPES, 150 mM NaCl, pH 7.3) at N/P ratios of 3 and 4. pCMV-hIFNβ was diluted with HBS to 0.04 mg/ml for all N/P ratios. The respective linear and random triconjugates were diluted with HBS to 0.0312 mg/ml (N/P 3) or 0.0208 mg/ml (N/P 4). The diluted delivery vectors were added to the diluted pCMV-hIFNβ and mixed by pipetting. Polyplexes were allowed to form for 30 minutes at room temperature. The final concentration of triconjugates and plasmid DNA in the polyplexes are shown below.

| N/P ratio | 3 | 4 |
|---|---|---|
| Conc of pCMV-hIFNβ (mg/ml) | 0.02 | 0.02 |
| Conc of random triconjugate (mg/ml) | 0.0078 | 0.0104 |
| Conc of linear triconjugate (mg/ml) | 0.0078 | 0.0104 |

The polyplexes were serially diluted and added to the cells to obtain the indicated final concentrations of the plasmid (0.25, 0.5, 1.0 and 2.0 μg/ml). The medium was collected 24 hours after the transfection and stored at −20° C. After thawing, the medium was analyzed by human IFNβ ELISA assay (LumiKine™ Xpress hIFN-β 2.0, Invivogen, Cat #luex-hifnbv2). The signal was detected using Luminoskan Ascent Microplate Luminometer (Thermo Scientific).

Delivery of plasmid pCMV-hIFNβ DNA using either the linear or random triconjugate vector results in high expression of human IFNβ protein (SEQ ID NO:23) in RENCAhEGFR M1 H cancer cells, in a dose-dependent manner, at the indicated N/P ratios. Increase in IFNβ expression and secretion was observed following transfection with polyplexes comprised of linear polyplex LPEI-l-[N₃:DBCO]-PEG₃₆-hEGF:pCMV-hIFNβ over transfection with polyplexes comprised of the random triconjugate and plasmid pCMV-hIFNβ (FIG. 17A and FIG. 17B).

These results demonstrate the targeted delivery of plasmid encoding human IFNβ by different delivery vectors to cancer cells that express high levels of EGFR. Efficient protein translation and secretion were evident for both delivery vectors. However, the linear triconjugate vector LPEI-l-[N₃:DBCO]-PEG₃₆-hEGF displayed a significant advantage over the random delivery vector.

1. A composition comprising a polyplex, wherein said polyplex comprise a conjugate and a nucleic acid, and
wherein said conjugate comprises:
a linear polyethyleneimine fragment comprising an alpha terminus and an omega terminus; wherein the alpha terminus of said polyethyleneimine fragment is an initiation residue;
a polyethylene glycol fragment comprising a first terminal end and a second terminal end; and
wherein the omega terminus of the polyethyleneimine fragment is connected to the first terminal end of the polyethylene glycol fragment by a divalent covalent linking group —Z—X¹—, wherein —Z—X¹— is not a single bond and —Z— is not an amide;
wherein the second terminal end of the polyethylene glycol fragment is connected to a targeting fragment by a divalent covalent linking moiety X², and
wherein said nucleic acid is a pharmaceutically active nucleic acid, wherein said pharmaceutically active nucleic acid is a nucleic acid that encodes a pharmaceutically active peptide or protein.

2. The composition of claim 1, wherein said conjugate is of the Formula I* or a pharmaceutically acceptable salt, solvate, hydrate, tautomer or enantiomer thereof 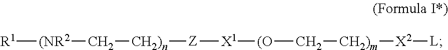
wherein
n is any integer between 1 and 1500;
m is any integer between 1 and 200;
R¹ is an initiation residue, wherein preferably R¹ is —H or —CH₃;
R² is independently —H or an organic residue, wherein at least 80%, preferably 90%, of said R² in said —(NR²—CH₂—CH₂)ₙ— is H;
X¹ and X² are independently divalent covalent linking moieties;
Z is a divalent covalent linking moiety wherein Z is not a single bond and Z is not —NHC(O)—;
L is a targeting fragment, wherein preferably said targeting fragment is capable of binding to a cell.

3. The composition of claim 1 or claim 2, wherein said conjugate is of the Formula I, or a pharmaceutically acceptable salt, solvate, hydrate, tautomer or enantiomer thereof: 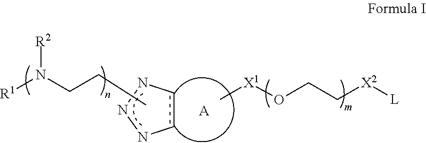
wherein: [symbol]
is a single bond or a double bond;
n is any integer between 1 and 1500;
m is any integer between 1 and 200;
R¹ is an initiation residue, wherein preferably R¹ is —H or —CH₃;
R² is independently —H or an organic residue, wherein at least 80%, preferably wherein at least 90%, of said R² in said —(NR²—CH₂—CH₂)ₙ— is H;
Ring A is a 5 to 10-membered cycloalkyl, cycloalkenyl, heterocycloalkyl, or heterocycloalkenyl, optionally substituted with one or more R^{A1}; R^{A1} is independently selected from C₁-C₆ alkyl, C₁-C₆ alkoxy, oxo, or halogen; or two R^{A1}, together with the atoms to which they are attached, can combine to form one or more fused C₆-C₁₀ aryl, C₅-C₆ heteroaryl, or C₃-C₆ cycloalkyl rings, wherein each fused aryl, heteroaryl, or cycloalkyl is optionally substituted with one or more R^{A2}; R^{A2} is independently selected from C₁-C₆ alkyl, C₁-C₆ alkoxy, halogen —SO₃H, or —OSO₃H;
X¹ is a divalent covalent linking moiety;
X² is a divalent covalent linking moiety; and
L is a targeting fragment, wherein preferably said targeting fragment is capable of binding to a cell.

4. The composition of any one of the claims 2 to 3, wherein said —(O—CH₂—CH₂)ₘ-moiety consists of a discrete number of repeating units m of 4 to 60, wherein preferably said discrete number m of repeating —(O—CH₂—CH₂)— units is 36.

5. The composition of any one of the claims 3 to 4, wherein said conjugate of Formula I is selected from:

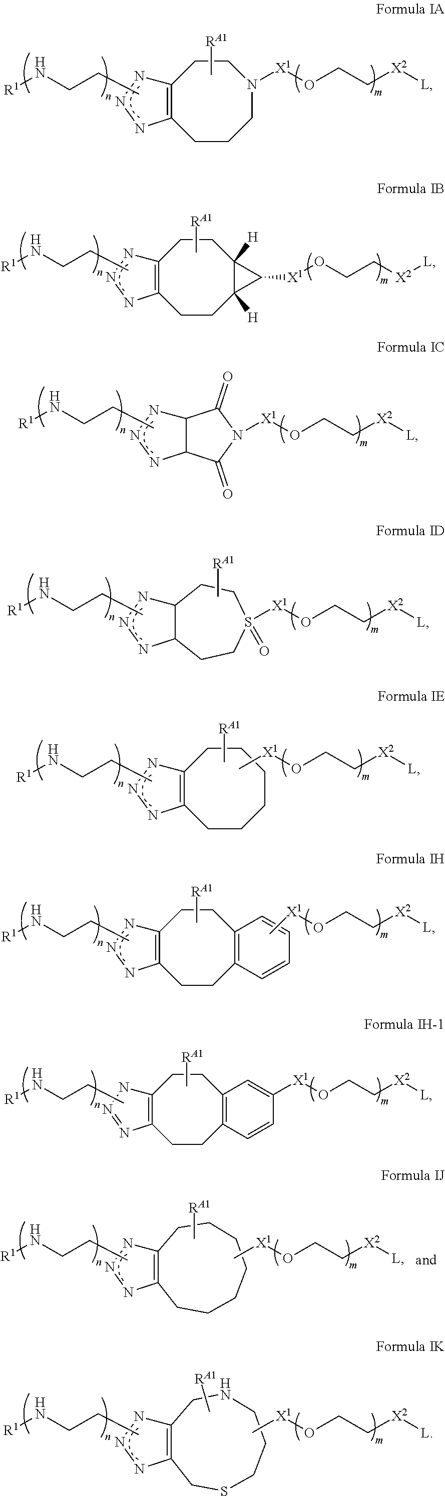

6. The composition of any one of the claims 3 to 5, wherein said conjugate of Formula I is selected from:

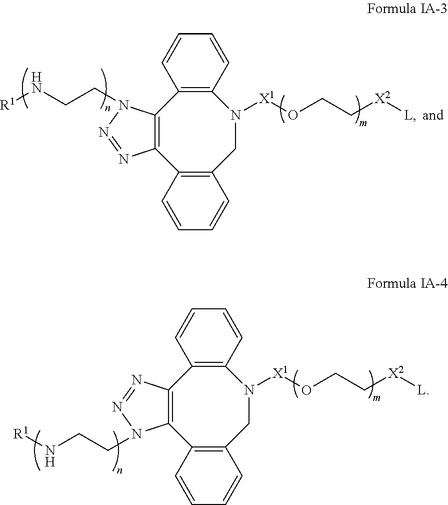

7. The composition of any one of the claims 3-6, wherein X¹ comprises a group selected from: 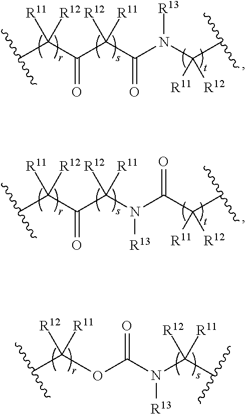
wherein:
r is independently, at each occurrence, 0-6, preferably 0, 1, 2, or 5;
s is independently, at each occurrence, 0-6, preferably 0, 2, 3, or 4;
t is independently, at each occurrence, 0-6, preferably 0, 1, 2, 4;
R¹¹ and R¹² are independently, at each occurrence, selected from —H and —C₁-C₂ alkyl; and
R¹³ is —H; preferably wherein the wavy line nearest to the integer “r” is a bond to Ring A and the wavy line nearest to the integer “s” or “t” is a bond to —[OCH₂—CH₂]ₘ—.

8. The composition of any one of claims 3-7, wherein X¹ is selected from: 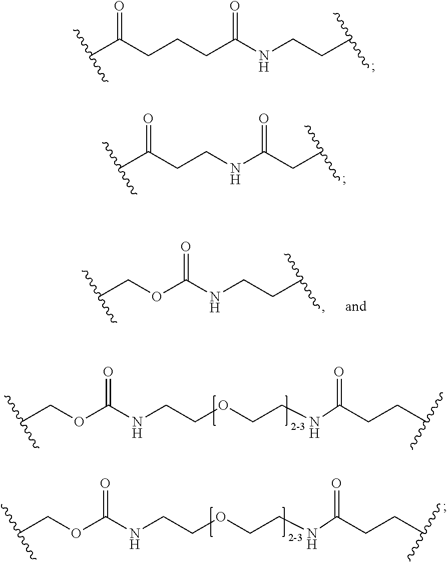
preferably wherein the wavy line on the left side is a bond to Ring A and the wavy line on the right side is a bond to —[OCH₂—CH₂]ₘ—.

9. The composition of any one of claims 3-8, wherein X² is selected from: 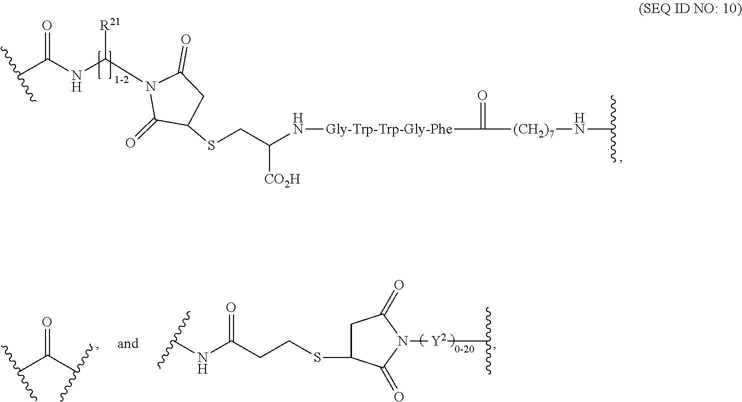
wherein each occurrence of Y² is independently selected from a chemical bond, —CR²¹R²²—, NR²³—, —O—, —S—, —C(O)—, an amino acid residue, a divalent phenyl moiety, a divalent carbocyle moiety, a divalent heterocycle moiety, and a divalent heteroaryl moiety, wherein each divalent phenyl and divalent heteroaryl is optionally substituted with one or more R²³, and wherein each divalent heterocycle moiety is optionally substituted with one or more R²⁴;
R²¹, R²², and R²³ are each independently, at each occurrence, —H, —SO₃H, —NH₂, —CO₂H, or C₁-C₆ alkyl, wherein each C₁-C₆ alkyl is optionally substituted with one or more —OH, oxo, —CO₂H, —NH₂, C₆-C₁₀ aryl, or 5 to 8-membered heteroaryl; and
R²⁴ is independently, at each occurrence, —H, —CO₂H, C₁-C₆ alkyl, or oxo; preferably 5 wherein the wavy line on the left side is a bond to —[OCH₂—CH₂]m- and the wavy line on the right side is a bond to L.

10. The composition of any of claims 3-9, wherein X² is selected from: 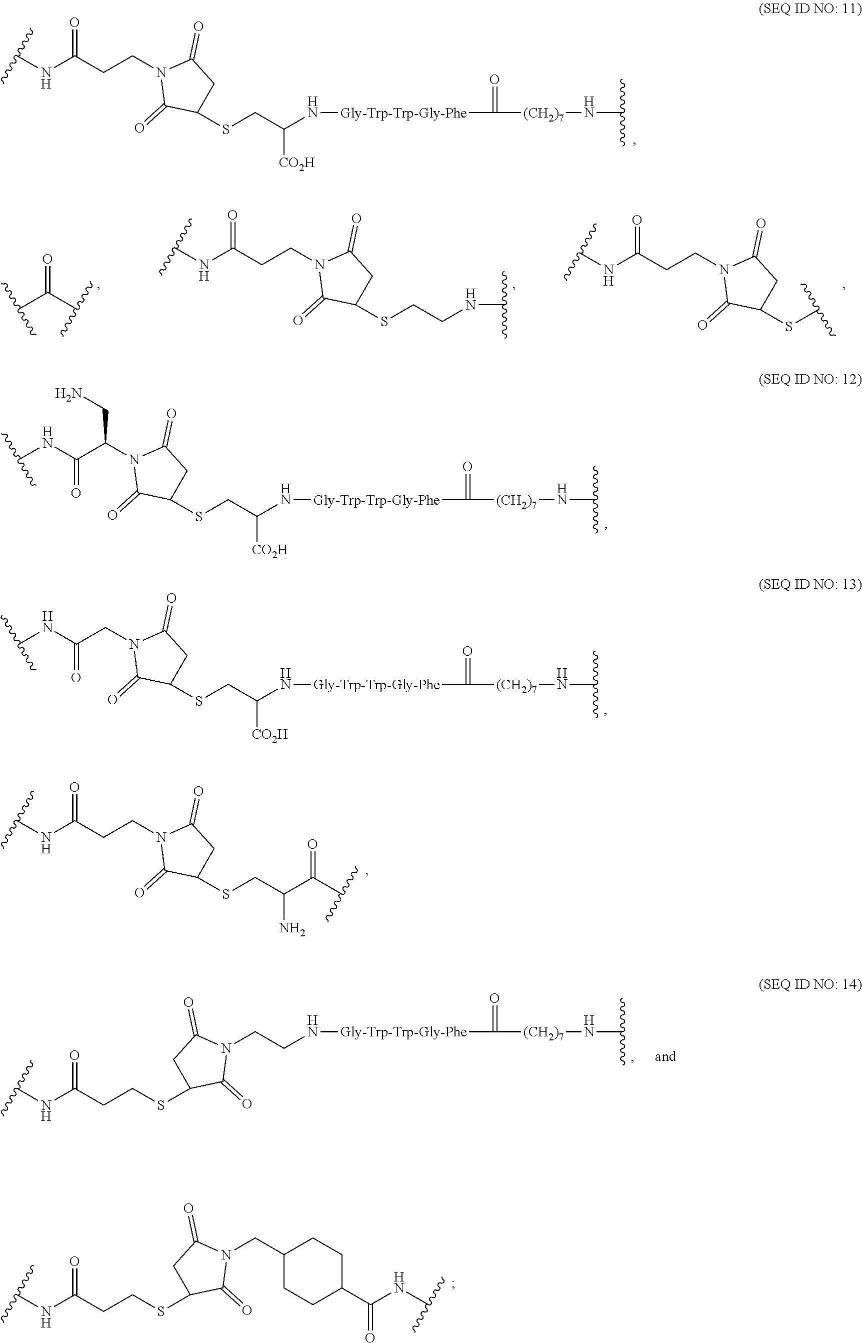
preferably wherein the wavy line on the left side is a bond to —[OCH₂—CH₂]m- and the wavy line on the right side is a bond to L.

11. The composition of any one of the preceding claims, wherein said targeting fragment L is capable of binding to a cell surface receptor, wherein said cell surface receptor is selected from a growth factor receptor, a cytokine receptor, a hormone receptor, an extracellular matrix protein, a transmembrane protein, a glycosylphosphatidylinositol (GPI) anchored membrane protein, a carbohydrate-binding integral membrane protein, a lectin, an ion channel, a G-protein coupled receptor, and an enzyme-linked receptor such as a tyrosine kinase-coupled receptor, wherein preferably said cell surface receptor is selected from an epidermal growth factor receptor (EGFR), human epidermal growth factor receptor 2 (HER2), prostate specific membrane antigen (PSMA), an insulin-like growth factor 1 receptor (IGF1R), a vascular endothelial growth factor receptor (VEGFR), a platelet-derived growth factor receptor (PDGFR), an asialoglycoprotein receptor (ASGPr) and a fibroblast growth factor receptor (FGFR).

12. The composition of any one of the preceding claims, wherein said targeting fragment L is selected from an EGFR targeting fragment, preferably human EGF (hEGF); a PSMA targeting fragment, preferably the DUPA residue; an anti-HER2 peptide, preferably an anti-HER2 antibody or affibody; folic acid; methotrexate; a somatostatin receptor-targeting fragment, preferably somatostatin and/or octreotide; an integrin-targeting fragment, preferably an arginine-glycine-aspartic acid (RGD)-containing fragment; a low pH insertion peptide; an ASGPr targeting fragment, preferably asialoorosomucoid; an insulin-receptor targeting fragment, preferably insulin; a mannose-6-phosphate receptor targeting fragment, preferably mannose-6-phosphate; a mannose-receptor targeting fragment, preferably mannose; a Sialyl Lewis^{x} antigen targeting fragments, preferably E-selectin; a sigma-2 receptor agonist, preferably N,N-dimethyltryptamine (DMT), sphingolipid-derived amine, and/or steroid, more preferably progesterone; a p32-targeting ligand, preferably anti-p32 antibody or p32-binding LyP-1 tumor-homing peptide; a Trop-2 targeting fragment, preferably an anti-Trop-2 antibody and/or antibody fragment; insulin-like growth factor 1; vascular endothelial growth factor; platelet-derived growth factor; and fibroblast growth factor.

13. The composition of any one of the preceding claims, wherein said conjugate is selected from Compound 1a, Compound 1b, Compound 4a, Compound 4b, Compound 7a, Compound 7b, Compound 10a, Compound 10b, Compound 14, Compound 17a, Compound 17b, Compound 18, Compound 19, Compound 22a, Compound 22b, Compound 28a, Compound 28b, Compound 31a, Compound 31b, Compound 38a, Compound 38b, Compound 43, Compound 47a, Compound 47b, Compound 51a, Compound 51b, Compound 56a, Compound 56b, Compound 62a, Compound 62b, Compound 70a, Compound 70b, Compound 72a, Compound 72b, Compound 75a, Compound 75b, Compound 78a Compound 78b, Compound 81, Compound 82a Compound 82b and/or Compound 83.

14. The composition of any one of the preceding claims, wherein said nucleic acid is a RNA, and wherein said RNA is a messenger RNA (mRNA).

15. The composition of any one of the preceding claims, wherein said nucleic acid is a DNA, wherein said DNA is a plasmid DNA.

16. The composition of any one of the preceding claims, wherein said pharmaceutically active peptide or protein is selected from a cytokine, a growth factor, a hormone, an enzyme, a tumor antigen, a viral antigen, bacterial antigen, an autoantigen, or an allergen, wherein preferably said pharmaceutically active peptide or protein is a cytokine selected from an interleukin, an interferon and a chemokine.


SEQUENCE LISTING (27 sequences)

SEQ ID NO: 1 - protein, 7 aa, synthetic construct
  SITE 1: note = 8-aminooctanoic acid
XFGWWGC

SEQ ID NO: 2 - protein, 4 aa, synthetic construct
WWGF

SEQ ID NO: 3 - protein, 6 aa, synthetic construct
  SITE 4: note = diaminopropionic acid
  SITE 1: note = 8-aminooctanoic acid
XFFXDC

SEQ ID NO: 4 - protein, 7 aa, synthetic construct
  SITE 1: note = 8-aminooctanoic acid
  BINDING 1: note = DUPA residue
XFGWWGC

SEQ ID NO: 5 - protein, 7 aa, synthetic construct
  SITE 1: note = 8-aminooctanoic acid
  BINDING 1: note = DUPA residue
  BINDING 7: note = Maleimide
XFGWWGC

SEQ ID NO: 6 - protein, 13 aa, synthetic construct
CYHWYGYTPQNVI

SEQ ID NO: 7 - protein, 53 aa, Homo sapiens
NSDSECPLSHDGYCLHDGVCMYIEALDKYACNCVVGYIGERCQYRDLKWWELR

SEQ ID NO: 8 - protein, 82 aa, synthetic construct
MGSSHHHHHHSSGLVPRGSHMASAEAKYAKEMWAAWEEIRNLPNLTGWQMTAFIAKLVDDPSQSSELLSEAKKLNDSQAPKC

SEQ ID NO: 9 - protein, 12 aa, synthetic construct
YHWYGYTPQNVI

SEQ ID NO: 10 - protein, 5 aa, synthetic construct
  BINDING 1: note = W1
  BINDING 5: note = W2
GWWGF

SEQ ID NO: 11 - protein, 5 aa, synthetic construct
  BINDING 1: note = W3
  BINDING 5: note = W4
GWWGF

SEQ ID NO: 12 - protein, 5 aa, synthetic construct
  BINDING 1: note = W5
  BINDING 5: note = W6
GWWGF

SEQ ID NO: 13 - protein, 5 aa, synthetic construct
  BINDING 1: note = W7
  BINDING 5: note = W8
GWWGF

SEQ ID NO: 14 - protein, 5 aa, synthetic construct
  BINDING 1: note = W9
  BINDING 5: note = W10
GWWGF

SEQ ID NO: 15 - skipped

SEQ ID NO: 16 - RNA, 936 nt, synthetic construct
  source 1..936: mol_type = other RNA
atgaccagcaaggtgtacgaccccgagcagcggaagcggatgatcaccggcccccagtggtgggcccggtgcaagcagatgaacgtgctggacagcttcatcaactactacgacagcgagaagcacgccgagaacgccgtgatcttcctgcacggcaacgccgccagcagctacctgtggcggcacgtggtgccccacatcgagcccgtggcccggtgcatcatccccgacctgatcggcatgggcaagagcggcaagagcggcaacggcagctaccggctgctggaccactacaagtacctgaccgcctggttcgagctgctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggcccgacatcgaggaggacatcgccctgatcaagagcgaggagggcgagaagatggtgctggagaacaacttcttcgtggagaccatgctgcccagcaagatcatgcggaagctggagcccgaggagttcgccgcctacctggagcccttcaaggagaagggcgaggtgcggcggcccaccctgagctggccccgggagatccccctggtgaagggcggcaagcccgacgtggtgcagatcgtgcggaactacaacgcctacctgcgggccagcgacgacctgcccaagatgttcatcgagagcgaccccggcttcttcagcaacgccatcgtggagggcgccaagaagttccccaacaccgagttcgtgaaggtgaagggcctgcacttcagccaggaggacgcccccgacgagatgggcaagtacatcaagagcttcgtggagcgggtgctgaagaacgagcagtga

SEQ ID NO: 17 - RNA, 464 nt, synthetic construct
  source 1..464: mol_type = other RNA
atgtacaggatgcaactcctgtcttgcattgcactaagtcttgcacttgtcacaaacagtgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgagc

SEQ ID NO: 18 - RNA, 564 nt, synthetic construct
  source 1..564: mol_type = other RNA
atgaccaacaagtgcctgctgcagatcgccctcctcctctgtttcagcaccaccgctctgagcatgagctacaacctgctgggcttcctgcagcggagcagcaacttccagtgccagaagctcctgtggcagctgaacggaagactggagtattgcctgaaggaccggatgaacttcgacatccccgaggagatcaagcagctgcagcagttccagaaggaggacgcagccctgaccatctacgagatgctgcagaacatcttcgccatcttcaggcaggatagcagcagcaccggttggaacgagaccatcgtggagaacctgctggccaacgtgtaccaccagatcaaccacctgaagaccgtgctggaggagaagctggagaaggaggacttcaccaggggcaagctgatgagcagcctgcacctgaagcggtactacggacggatcctgcactacctgaaggccaaggagtacagccactgcgcttggacaattgtgcgggtggagatcctgcggaacttctacttcatcaaccggctgaccggctacctgaggaactag

SEQ ID NO: 19 - RNA, 501 nt, synthetic construct
  source 1..501: mol_type = other RNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 20 - RNA, 582 nt, synthetic construct
  source 1..582: mol_type = other RNA
atgggcgtgcacgagtgccccgcctggctgtggctgctgctgagcctgctgagcctgcccctgggcctgcccgtgctgggcgcccccccccggctgatctgcgacagccgggtgctggagcggtacctgctggaggccaaggaggccgagaacatcaccaccggctgcgccgagcactgcagcctgaacgagaacatcaccgtgcccgacaccaaggtgaacttctacgcctggaagcggatggaggtgggccagcaggccgtggaggtgtggcagggcctggccctgctgagcgaggccgtgctgcggggccaggccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccggaccggcgaccggtga

SEQ ID NO: 21 - RNA, 585 nt, synthetic construct
  source 1..585: mol_type = other RNA
atgggagcagacgacgtggtggatagcagcaagagcttcgtgatggagaacttcagcagctaccacggcaccaagccaggatacgtggacagcatccagaagggcatccagaagcccaagagcggaacccagggcaactacgacgacgattggaagggcttctacagcaccgacaacaagtacgacgccgccggatacagcgtggataacgagaatcctctgagcggcaaagccggaggagtggtgaaagtgacctacccaggcctgaccaaggtgctggctctgaaggtggacaacgccgagaccatcaagaaggagctgggcctgagcctgaccgaacctctgatggagcaggtcggaaccgaggagttcatcaagcgcttcggcgacggagctagcagagtggtgctgtctctgccttttgccgagggaagcagcagcgtggagtacatcaacaattgggagcaggccaaggccctgagcgtggagctggagatcaacttcgagaccaggggcaagagaggccaggacgcaatgtacgagtacatggcccaggcttgcgcaggaaacagagtgagaaggtag

SEQ ID NO: 22 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 24 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 25 - protein, 225 aa, synthetic construct
MLVRGYVVSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRR

SEQ ID NO: 26 - DNA, 464 bp, synthetic construct
  source 1..464: mol_type = other DNA
atgtacaggatgcaactcctgtcttgcattgcactaagtcttgcacttgtcacaaacagtgcacctacttcaagttctacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgagc

SEQ ID NO: 27 - DNA, 564 bp, synthetic construct
  source 1..564: mol_type = other DNA
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga